(19) United States
(12) Patent Application Publication
(10) Pub. No.: US 2024/0059689 A1
(43) Pub. Date: Feb. 22, 2024

(54) PURINE DERIVATIVES AS ANTICANCER AGENTS

(71) Applicants: Tango Therapeutics, Inc., Boston, MA (US); Medivir AB, Huddinge (SE)

(72) Inventors: Scott Throner, Newton, MA (US); Daniel Jönsson, Huddinge (SE)

(21) Appl. No.: 18/468,385

(22) Filed: Sep. 15, 2023

Related U.S. Application Data
(63) Continuation of application No. PCT/US2022/020700, filed on Mar. 17, 2022.
(60) Provisional application No. 63/162,460, filed on Mar. 17, 2021.

(51) Int. Cl. C07D 473/30; C07D 473/34

(52) U.S. Cl. CPC C07D 473/30; C07D 473/34

(57) ABSTRACT

Compounds are provided according to Formula (I)

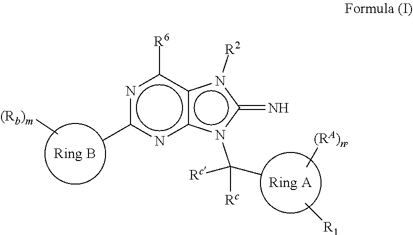

and pharmaceutically acceptable salts, hydrates, solvates, prodrugs, tautomers and stereoisomers, as well as pharmaceutical compositions, wherein Ring B, Ring A, R^{A}, R^{b}, R^{c}, R^{c′}, R¹, R², R⁶, m and n are as defined herein. The compounds disclosed herein are contemplated to be useful for the prevention and treatment of a variety of conditions.

### CROSS-REFERENCE TO RELATED APPLICATION

This application is a continuation of International (PCT) Patent Application No. PCT/US2022/020700, filed Mar. 17, 2022, which claims the benefit of and priority to U.S. provisional patent application No. 63/162,460, filed Mar. 17, 2021, the contents of each of which are incorporated herein by reference in their entirety.

### BACKGROUND

Ubiquitin is a small, highly conserved protein composed of 76 amino acids that is post-transcriptionally attached to target proteins, including itself, via a concerted three-step enzymatic reaction. This covalent linkage or isopeptide bond primarily occurs between the C-terminal glycine of ubiquitin and the F-amino group of lysine residue(s) on the target protein (Pickart, C.

M., Annu. Rev. Biochem., 2001: 503-33). The functional consequence of ubiquitination is determined by the number and linkage topology of ubiquitin molecules conjugated to the target protein. For example, proteins exhibiting Lys48-linked polyubiquitin chains are generally targeted to the proteasome for degradation, while monoubiquitination or polyubiquitin chains linked through other lysines regulate several non-proteolytic functions, including cell cycle regulation (Nakayama, K. I. et al., Nat. rev. Cancer, 6(5): 369-81 (2006)), DNA repair (Bergink, S., et al., Nature 458(7237): 461-7 (2009)), transcription (Conaway, R. C, et al., Science 296(5571): 1254-8 (2002)), and endocytosis (Mukhopadhyay, D., et al., Science 315(5809): 201-5 (2007)). Similar to other posttranslational modifications, ubiquitination is a reversible process counteracted by a family of enzymes known as deubiquitinases (DUBs). These enzymes are cysteine proteases or metalloproteases that hydrolyze the ubiquitin isopeptide bond (Komander, D., et al., Nat. Rev. Mol. Cell Biol. 10(8): 550-63 (2007)). The human genome encodes close to 100 DUBs.

DUBs and their substrate proteins are often deregulated in cancers. Targeting specific DUB family members may result in antitumor activity by enhancing the ubiquitination and subsequent degradation of oncogenic substrates, involved in tumor growth, survival, differentiation and maintenance of the tumor microenvironment. (Hussain, S. et. al., “DUBs and cancer: The role of deubiquitinating enzymes as oncogenes, non-oncogenes and tumor suppressors.” Cell Cycle 8, 1688-1697 (2009)). Consequently, several members of the DUB family have been implicated in processes related to human disease, including cancer and neurodegeneration. Among them, USP1 (ubiquitin-specific protease 1) has gained increased interest as a novel therapeutic target given its roles in the DNA damage response.

USP1 is a cysteine isopeptidase of the USP subfamily of deubiquitinases (DUBs). (Nijman, S. M. B., et al. “The deubiquitinating enzyme USP1 regulates the Fanconi anemia pathway. Mol. Cell 17, 331-339 (2005)) Full-length human USP1 is composed of 785 amino acids, including a catalytic triad composed of Cys90, His593 and Asp751. (Villamil, M. A., et al, “Serine phosphorylation is critical for the activation of ubiquitin-specific protease 1 and its interaction with WD40-repeat protein UAF1.” Biochem. 51, 9112-9113 (2012)). USP1 is relatively inactive on its own and full enzymatic activity is achieved only when bound in a heterodimeric complex with USP1 Associated Factor 1 (UAF1), which also binds to and regulates the activity of USP12 and USP46. (Cohn, M. A., et al, “A UAF1-Containing Multisubunit Protein Complex Regulates the Fanconi Anemia Pathway.” Mol. Cell 28, 786-797 (2007)).

USP1 deubiquitinates a variety of cellular targets involved in different processes related to cancer. For example, USP1 deubiquitinates PCNA (proliferating cell nuclear antigen), a key protein in translesion synthesis (TLS), and FANCI/FANCD2 (Fanconi anemia group complementation group D2), a key protein complex in the Fanconi anemia (FA) pathway. (Nijman, S. M. B. et al “The deubiquitinating enzyme USP1 regulates the Fanconi anemia pathway.” Mol Cell 17, 331-339 (2005); Huang, T. T. et al, “Regulation of monoubiquitinated PCNA by DUB autocleavage.” Nat. Cell Biol 8, 339-347 (2006)). These DNA damage response (DDR) pathways are essential for repair of DNA damage, including those induced by DNA cross-linking agents such as cisplatin, mitomycin C (MMC), diepoxybutane, ionizing radiation and ultraviolet radiation. In addition, USP1 promotes cancer cell stem maintenance by increasing inhibitor of protein binding (ID) protein stability. Thus, USP1 inhibition may antagonize cancer cell growth by inducing cell cycle arrest and decreasing cancer stem cell maintenance via a decrease in ID protein stability. (Williams, S. A. et al, “USP1 deubiquitinates ID proteins to preserve a mesenchymal stem cell program in osteosarcoma.” Cell 146: 918-30 (2011); Lee, J. K. et al, “USP1 targeting impedes GBM growth by inhibiting stem cell maintenance and radioresistance.” Neuro Oncol. 18: 37-47 (2016)).

The compounds GW7647 and Pimozide have been described as inactivators of USP1. However, both compounds are limited by potency and off-target pharmacology, in part because both of them have noticeable activity against unrelated targets. Another small molecule inhibitor of USP1, C527, which was reported by D'Andrea et al. in WO2011/137320, sensitizes cells to both the crosslinking agent, mitomycin C, and the topoisomerase 1 inhibitor, camptothecin. However, C527 shows low micromolar inhibition of related USPs as well as dissimilar DUBs (i.e., UCHL-1 and UCHL-3). Another small molecule USP1-UAF1 inhibitor (ML323) has been more recently disclosed (Dexheimer et al, J. Med. Chem. 2014, 57, 8099-8110; Liang et al, Nature Chem. Bio. 2015, 10, 298-304; U.S. Pat. No. 9,802,904 B2). Additional USP1 inhibitors have also been described in WO2017087837, WO2020132269, WO2020139988, and WO2021163530.

The foregoing shows that there exists an unmet need for new selective inhibitors of USP1, and in addition, that inhibition of USP1 with small molecule inhibitors has the potential to be a treatment for cancers and other disorders. For these reasons, there remains a considerable need for potent small molecule inhibitors of USP1.

### SUMMARY

In one embodiment, provided is a compound of Formula (I) or a pharmaceutically acceptable salt, hydrate, solvate, prodrug, stereoisomer, or tautomer thereof wherein:

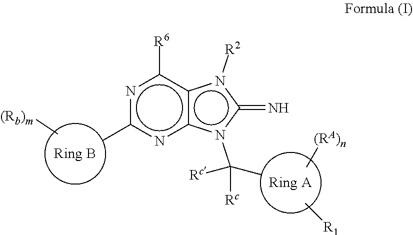

Ring B is a 5-6 member monocyclic aryl or heteroaryl;
Ring A is selected from C₆-C₁₀ aryl, 5-10 membered heteroaryl, —C₃-C₁₀ cycloalkyl, and 3-10 membered heterocyclyl;
R¹ is an optionally substituted 5-10 membered heteroaryl or an optionally substituted 3-10 membered heterocyclyl;
R² is selected from H, —C₁-C₆ alkyl, —C₁-C₆ haloalkyl, —C₁-C₆ heteroalkyl, —C₁-C₆ hydroxyalkyl, —C₃-C₁₀ cycloalkyl and arylalkyl, wherein each hydrogen of the alkyl, haloalkyl, heteroalkyl, hydroxylalkyl and arylalkyl can be independently replaced with a deuterium atom;
R⁶ is selected from H, -D, halo, —CN, —C₁-C₆ alkyl, —C₁-C₆ alkynyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, 6-10 member heteroaryl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, and —OC(═O)N(R^{a6})₂, wherein each alkyl, alkynyl, cycloalkyl, heterocyclyl, aryl, heteroaryl, arylalkyl and heteroarylalkyl is optionally substituted at any available position;
each R^{a6} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl;
each R^{A} is independently selected from -D, halo, —CN, —C₁-C₆ alkyl, —C₁-C₆ haloalkyl, —C₁-C₆ hydroxyalkyl, —C₃-C₁₀ cycloalkyl, —OR^{A1}, —N(R^{A1})₂;
each R^{A1} is independently selected from H, —C₁-C₆ alkyl, —C₁-C₆ haloalkyl and C₃-C₉ cycloalkyl;
each R^{b} is independently selected from -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{b1}, —N(R^{b1})₂, —C(═O)R^{b1}, —C(═O)OR^{b1}, —NRIC(═O)R^{b1}, —NRIC(═O)OR^{b1}, —C(═O)N(R^{b1})₂, —OC(═O)N(R^{b1})₂, —S(═O)R^{b1}, —S(═O)₂R^{b1}, —SR¹, —S(═O)(═NR^{b1})R^{b1}, —NR^{b1}S(═O)₂R^{b1} and —S(═O)₂N(R^{b1})₂ or 2 R^{b} together with the atoms to which they are attached form a 4-7 member carbocyclyl or a 4-7 member heterocyclyl, wherein each alkyl, carbocylyl, cycloalkyl, heterocyclyl, aryl, heteroaryl, arylalkyl and heteroarylalkyl of R^{b} is optionally substituted at any available position;
each R^{b1} is independently selected from H, —C₁-C₆ alkyl (wherein each hydrogen can be independently replaced by deuterium), —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl;
each R^{c} and R^{c′} is independently selected from H, -D, —C₁-C₆ alkyl (e.g., -Me), —C₁-C₆ heteroalkyl and —C₁-C₆ haloalkyl or R^{c} and R^{c′} can be taken together with the atom to which they are attached to form a —C₃-C₉ cycloalkyl (e.g., cyclopropyl) or a carbonyl;
n is 0, 1, 2 or 3; and
m is 0, 1, 2 or 3.

In some embodiments, provided is a compound is of Formula (II)

wherein:

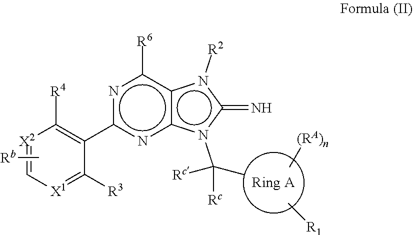

X¹ is selected from CH and N;
X² is selected from CH and N;
R³ is selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂ wherein each alkyl, cycloalkyl, heterocyclyl, aryl, heteroaryl, arylalkyl and heteroarylalkyl is optionally substituted at any available position;
R⁴ is selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂ wherein each alkyl, cycloalkyl, heterocyclyl, aryl, heteroaryl, arylalkyl and heteroarylalkyl is optionally substituted at any available position; and each R^{a3} and R^{a4} is independently selected from H, —C₁-C₆ alkyl (wherein each hydrogen can be replaced by deuterium), —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In some embodiments, provided are compounds selected from the compounds of Table 1, or a pharmaceutically acceptable salt, hydrate, solvate, prodrug, stereoisomer, or tautomer thereof.

In some embodiments, provided is a pharmaceutical composition comprising a compound as described herein or a pharmaceutically acceptable salt, hydrate, solvate, prodrug, stereoisomer, or tautomer thereof and a pharmaceutically acceptable carrier.

In some embodiments, the pharmaceutical composition comprises a second therapeutic agent.

In some embodiments, provided is a method for treating or preventing a disease or disorder associated with the inhibition of USP1 comprising administering to a patient in need thereof an effective amount of a compound of Formula (I) as described herein or a pharmaceutically acceptable salt, hydrate, solvate, prodrug, stereoisomer, or tautomer thereof.

In some embodiments, provided is a method of treating a disease or disorder associated with the inhibition of USP1 comprising administering to a patient in need thereof an effective amount of a compound of Formula (I) as described herein or a pharmaceutically acceptable salt, hydrate, solvate, prodrug, stereoisomer, or tautomer thereof.

In some embodiments, provided is a method for inhibiting USP1 comprising administering to a patient in need thereof an effective amount of a compound of Formula (I) as described herein or a pharmaceutically acceptable salt, hydrate, solvate, prodrug, stereoisomer, or tautomer thereof.

In some embodiments, provided is a method for treating or preventing cancer in a patient in need thereof comprising administering to the patient in need thereof an effective amount of a compound of Formula (I) as described herein or a pharmaceutically acceptable salt, hydrate, solvate, prodrug, stereoisomer, or tautomer thereof.

In some embodiments, provided is a method for treating cancer in a patient in need thereof comprising administering to the patient in need thereof an effective amount of a compound of Formula (I) as described herein or a pharmaceutically acceptable salt, hydrate, solvate, prodrug, stereoisomer, or tautomer thereof.

In some embodiments, provided is a method for treating or preventing a disease or disorder associated with DNA damage comprising administering to a patient in need of a treatment for diseases or disorders associated with DNA damage an effective amount of a compound of Formula (I) as described herein or a pharmaceutically acceptable salt, hydrate, solvate, prodrug, stereoisomer, or tautomer thereof. In some embodiments the disease is cancer.

In some embodiments, provided is a method for treating a disease or disorder associated with DNA damage comprising administering to a patient in need of a treatment for diseases or disorders associated with DNA damage an effective amount (e.g., a therapeutically effective amount) of a compound of Formula (I) as described herein or a pharmaceutically acceptable salt, hydrate, solvate, prodrug, stereoisomer, or tautomer thereof.

In some embodiments, provided is a method of inhibiting, modulating or reducing DNA repair activity exercised by USP1 comprising administering to a patient in need thereof an effective amount of a compound of Formula (I) as described herein or a pharmaceutically acceptable salt, hydrate, solvate, prodrug, stereoisomer, or tautomer thereof.

### DETAILED DESCRIPTION

The disclosure herein sets forth exemplary methods, parameters and the like. It should be recognized, however, that such description is not intended as a limitation on the scope of the present disclosure but is instead provided as a description of exemplary embodiments.

### Definitions

As used in the present disclosure, the following words and phrases are generally intended to have the meanings as set forth below unless expressly indicated otherwise or the context in which they are used indicates otherwise.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

The “enantiomeric excess” (“e.e.”) or “% enantiomeric excess” (“% e.e.”) of a composition as used herein refers to an excess of one enantiomer relative to the other enantiomer present in the composition. For example, a composition can contain 90% of one enantiomer, e.g., the S enantiomer, and 10% of the other enantiomer, i.e., the R enantiomer. e.e. =(90-10)/100=80%.

Thus, a composition containing 90% of one enantiomer and 10% of the other enantiomer is said to have an enantiomeric excess of 80%.

The “diastereomeric excess” (“d.e.”) or “% diastereomeric excess” (“% d.e.”) of a composition as used herein refers to an excess of one diastereomer relative to one or more different diastereomers present in the composition. For example, a composition can contain 90% of one diastereomer, and 10% of one or more different diastereomers.

d.e.=(90−10)/100=80%.

Thus, a composition containing 90% of one diastereomers and 10% of one or more different diastereomers is said to have a diastereomeric excess of 80%.

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

In a formula, [symbol] is a single bond where the stereochemistry of the moieties immediately attached thereto is not specified.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

The term “azido” refers to the radical —N₃.

“Aliphatic” refers to an alkyl, alkenyl, alkynyl, or carbocyclyl group, as defined herein.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Aralkyl” or “arylalkyl” is a subset of alkyl and aryl, as defined herein, and refers to an optionally substituted alkyl group substituted by an optionally substituted aryl group.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“Ci alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), ⁱPr (—CH(CH₃)₂), ⁿPr (—CH₂CH₂CH₃), ⁿBu (—CH₂CH₂CH₂CH₃), or ⁱBu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

When a range or number of carbons is provided for a particular alkylene group, it is understood that the range or number refers to the range or number of carbons in the linear carbon divalent chain. Alkylene groups may be substituted or unsubstituted with one or more substituents as described herein.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂-10 alkenyl.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₈ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroCi alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl. Exemplary heteroalkyl groups include: —CH₂OH, —CH₂OCH₃, —CH₂NH₂, —CH₂NH(CH₃), —CH₂N(CH₃)₂, —CH₂CH₂OH, —CH₂CH₂OCH₃, —CH₂CH₂NH₂, —CH₂CH₂NH(CH₃), —CH₂CH₂N(CH₃)₂.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group is substituted with one or more of groups selected from halo, C₁-C₈ alkyl, C₁-C₈ haloalkyl, cyano, hydroxy, C₁-C₈ alkoxy, and amino.

Examples of representative substituted aryls include the following

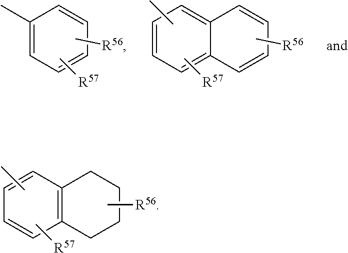

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁸SOR⁵⁹NR⁸SO₂R⁵⁹, COOalkyl, COOaryl, CONR⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵¹ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbons in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In some embodiments, a heteroaryl group is a bicyclic 8-12 membered aromatic ring system having ring carbon atoms and 1-6 ring heteroatoms provided in the aromatic ring system, wherein each heteroatom is independently selected from nitrogen, oxygen, and sulfur (“8-12 membered bicyclic heteroaryl”). In some embodiments, a heteroaryl group is an 8-10 membered bicyclic aromatic ring system having ring carbon atoms and 1-6 ring heteroatoms provided in the aromatic ring system, wherein each heteroatom is independently selected from nitrogen, oxygen, and sulfur (“8-10 membered bicyclic heteroaryl”). In some embodiments, a heteroaryl group is a 9-10 membered bicyclic aromatic ring system having ring carbon atoms and 1-6 ring heteroatoms provided in the aromatic ring system, wherein each heteroatom is independently selected from nitrogen, oxygen, and sulfur (“9-10 membered bicyclic heteroaryl”). Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

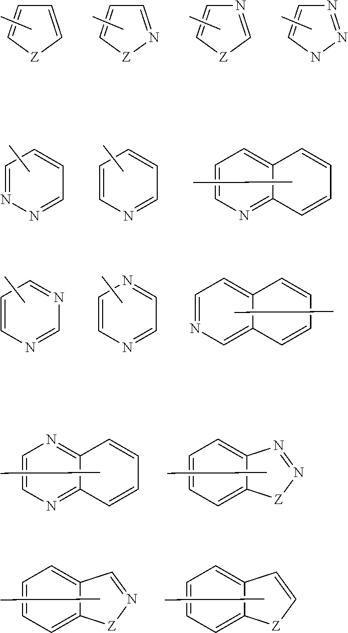

wherein each Z is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

In the structures described herein, a substituent attached to a polycyclic (e.g., bicyclic or tricyclic) cycloalkyl, heterocyclyl, aryl or heteroaryl with a bond that spans two or more rings is understood to mean that the substituent can be attached at any position in each of the rings.

“Heteroaralkyl” or “heteroarylalkyl” is a subset of “alkyl” and refers to an alkyl group substituted by a heteroaryl group, wherein the point of attachment is on the alkyl moiety.

The term “carbocyclyl” or “carbocyclic” refers to a radical of a non-aromatic monocyclic, bicyclic, or tricyclic or polycyclic hydrocarbon ring system having from 3 to 14 ring carbon atoms (“C₃₋₁₄ carbocyclyl”) and zero heteroatoms in the non-aromatic ring system. Carbocyclyl groups include fully saturated ring systems (e.g., cycloalkyls), and partially saturated ring systems. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

The term “cycloalkyl” as employed herein includes saturated cyclic, bicyclic, tricyclic, or polycyclic hydrocarbon groups having 3 to 14 carbons containing the indicated number of rings and carbon atoms (for example a C₃-C₁₄ monocyclic, C₄-C₁₄ bicyclic, C₅-C₁₄ tricyclic, or C₆-C₁₄ polycyclic cycloalkyl). In some embodiments “cycloalkyl” is a monocyclic cycloalkyl. In some embodiments, a monocyclic cycloalkyl has 3-14 ring carbon atoms. (“C₃₋₁₄ monocyclic cycloalkyl”). In some embodiments, a monocyclic cycloalkyl group has 3 to 10 ring carbon atoms (“C₃₋₁₀ monocyclic cycloalkyl”). In some embodiments, a monocyclic cycloalkyl group has 3 to 8 ring carbon atoms (“C₃₋₈ monocyclic cycloalkyl”). In some embodiments, a monocyclic cycloalkyl group has 3 to 6 ring carbon atoms (“C₃₋₆ monocyclic cycloalkyl”). In some embodiments, a monocyclic cycloalkyl group has 4 to 6 ring carbon atoms (“C₄₋₆ monocyclic cycloalkyl”). In some embodiments, a monocyclic cycloalkyl group has 5 to 6 ring carbon atoms (“C₅₋₆ monocyclic cycloalkyl”). In some embodiments, a monocyclic cycloalkyl group has 5 to 10 ring carbon atoms (“C₅₋₁₀ monocyclic cycloalkyl”). Examples of monocyclic C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄).

Examples of C₃₋₈ cycloalkyl groups include the aforementioned C₃₋₆ cycloalkyl groups as well as cycloheptyl (C₇) and cyclooctyl (C₈).

In some embodiments “cycloalkyl” is a bicyclic cycloalkyl. In some embodiments, a bicyclic cycloalkyl has 4-14 ring carbon atoms. (“C₄₋₁₄ bicyclic cycloalkyl”). In some embodiments, a bicyclic cycloalkyl group has 4 to 12 ring carbon atoms (“C₄₋₁₂ bicyclic cycloalkyl”). In some embodiments, a bicyclic cycloalkyl group has 4 to 10 ring carbon atoms (“C₄₋₁₀ bicyclic cycloalkyl”). In some embodiments, a bicyclic cycloalkyl group has 5 to 10 ring carbon atoms (“C₅₋₁₀ bicyclic cycloalkyl”). In some embodiments, a bicyclic cycloalkyl group has 6 to 10 ring carbon atoms (“C₆₋₁₀ bicyclic cycloalkyl”). In some embodiments, a bicyclic cycloalkyl group has 8 to 10 ring carbon atoms (“C₈₋₁₀ bicyclic cycloalkyl”). In some embodiments, a bicyclic cycloalkyl group has 7 to 9 ring carbon atoms (“C₇₋₉ bicyclic cycloalkyl”). Examples of bicyclic cycloalkyls include bicyclo[1.1.0]butane (C₄), bicyclo[1.1.1]pentane (C₅), spiro[2.2] pentane (C₅), bicyclo[2.1.0]pentane (C₅), bicyclo[2.1.1]hexane (C₆), bicyclo[3.1.0]hexane (C₆), spiro[2.3] hexane (C₆), bicyclo[2.2.1]heptane (norbornane) (C₇), bicyclo[3.2.0]heptane (C₇), bicyclo[3.1.1]heptane (C₇), bicyclo[3.1.1]heptane (C₇), bicyclo[4.1.0]heptane (C₇), spiro[2.4] heptane (C₇), spiro [3.3]heptane (C₇), bicyclo[2.2.2]octane (C₈), bicyclo[4.1.1]octane (C₈)octahydropentalene (C₈), bicyclo[3.2.1]octane (C₈), bicyclo[4.2.0]octane (C₈), spiro[2.5]octane (C₈), spiro[3.4]octane (C₈), bicyclo[3.3.1]nonane (C₉), octahydro-1H-indene (C₉), bicyclo[4.2.1]nonane (C₉), spiro[3.5]nonane (C₉), spiro[4.4]nonane (C₉), bicyclo[3.3.2]decane (C₁₀), bicyclo[4.3.1]decane (C₁₀), spiro[4.5]decane (C₁₀), bicyclo[3.3.3]undecane (C₁₁), decahydronaphthalene (C₁₀), bicyclo[4.3.2]undecane (C₁₁), spiro[5.5]undecane (C₁₁) and bicyclo[4.3.3]dodecane (C₁₂).

In some embodiments “cycloalkyl” is a tricyclic cycloalkyl. In some embodiments, a tricyclic cycloalkyl has 6-14 ring carbon atoms. (“C₆₋₁₄ tricyclic cycloalkyl”). In some embodiments, a tricyclic cycloalkyl group has 8 to 12 ring carbon atoms (“C₈₋₁₂ tricyclic cycloalkyl”). In some embodiments, a tricyclic cycloalkyl group has 10 to 12 ring carbon atoms (“C₁₀-12 tricyclic cycloalkyl. Examples of tricyclic cycloalkyls include adamantine (C₁₂).

Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Nitrogen-containing heterocyclyl” group means a 4- to 7-membered non-aromatic cyclic group containing at least one nitrogen atom, for example, but without limitation, morpholine, piperidine (e.g., 2-piperidinyl, 3-piperidinyl and 4-piperidinyl), pyrrolidine (e.g., 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(═O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), C(═O)—C₁-C₈ alkyl, —C(═O)—(CH₂)ₜ(C₆-C₁₀ aryl), —C(═O)—(CH₂)ₜ(5-10 membered heteroaryl), —C(═O)—(CH₂)ₜ(C₃-C₁₀ cycloalkyl), and —C(═O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

The term aminoalkyl refers to a substituted alkyl group wherein one or more of the hydrogen atoms are independently replaced by an —NH₂ group.

The term hydroxyalkyl refers to a substituted alkyl group wherein one or more of the hydrogen atoms are independently replaced by an —OH group.

The terms “alkylamino” and “dialkylamino” refer to —NH(alkyl) and —N(alkyl)₂ radicals respectively. In some embodiments the alkylamino is a-NH(C₁-C₄ alkyl). In some embodiments the alkylamino is methylamino, ethylamino, propylamino, isopropylamino, n-butylamino, iso-butylamino, sec-butylamino or tert-butylamino. In some embodiments the dialkylamino is —N(C₁-C₆ alkyl)₂. In some embodiments the dialkylamino is a dimethylamino, a methylethylamino, a diethylamino, a methylpropylamino, a methylisopropylamino, a methylbutylamino, a methylisobutylamino or a methyltertbutylamino.

The term “aryloxy” refers to an —O-aryl radical. In some embodiments the aryloxy group is phenoxy.

The term “haloalkoxy” refers to alkoxy structures that are substituted with one or more halo groups or with combinations thereof. For example, the term “fluoroalkoxy” includes haloalkoxy groups, in which the halo is fluorine. In some embodiments haloalkoxy groups are difluoromethoxy and trifluoromethoxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e., with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe2.

“Amino” refers to the radical —NH₂.

“Oxo group” refers to —C(═O)—.

“Substituted amino” refers to an amino group of the formula —N(R³⁸)₂ wherein R³¹ is hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R³⁸ is not a hydrogen. In certain embodiments, each R³¹ is independently selected from hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³¹ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

In certain embodiments, the substituent present on the nitrogen atom is a nitrogen protecting group (also referred to herein as an “amino protecting group”). Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. Nitrogen protecting groups include, but are not limited to, —OH, —OR^{aa}, —N(R^{cc})₂, —C(═O)R^{aa}, —C(═O)N(R^{cc}), —CO₂R^{aa}, —SO₂R^{aa}, —C(═NR^{cc})R^{aa}, —C(═NR^{cc})OR^{aa}, —C(═NR^{cc})N(R^{cc}), —SO₂N(R^{cc}), —SO₂R^{c}, —SO₂OR^{cc}, —SOR^{a}, —C(═S)N(R^{cc})₂, —C(═O)SR^{cc}, —C(═S)SR^{cc}, —C₁₋₁₀ alkyl (e.g., aralkyl, heteroaralkyl), —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein;
each instance of R^{aa} is, independently, selected from —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂—CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X is a counterion.
each instance of R^{cc} is, independently, selected from hydrogen, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{f})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NRe)R^{ee}, —OC(═NR^{ff})OR^{ee}, —C(═NR^{ff})N(R^{ff})₂, —OC(═NR^{ff})N(R^{ff})₂, —NRC(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from —C₁₋₆ alkyl, —C₁₋₆ perhaloalkyl, —C₂₋₆ alkenyl, —C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(Ci-6 alkyl)₂, —OP(═O)(OC₁₋₆ alkyl)₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R⁹⁹ substituents can be joined to form ═O or ═S; wherein X⁻ is a counterion.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{b})₂)₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃+X, —P(ORC)₂, —P(OR^{cc})₃⁺X⁻, —P(═O)(R′)₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “leaving group” is given its ordinary meaning in the art of synthetic organic chemistry and refers to an atom or a group capable of being displaced by a nucleophile. Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In certain embodiments, the leaving group is halogen, alkanesulfonyloxy, arenesulfonyloxy, diazonium, alkyl diazenes, aryl diazenes, alkyl triazenes, aryl triazenes, nitro, alkyl nitrate, aryl nitrate, alkyl phosphate, aryl phosphate, alkyl carbonyl oxy, aryl carbonyl oxy, alkoxcarbonyl oxy, aryoxcarbonyl oxy ammonia, alkyl amines, aryl amines, hydroxyl group, alkyloxy group, or aryloxy. In some cases, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

“Carboxy” refers to the radical —C(═O)OH.

“Cyano” refers to the radical —CN.

“Halo” or “halogen” refers to fluoro (F), chloro (C₁), bromo (Br), and iodo (I). In certain embodiments, the halo group is either fluoro or chloro.

“Haloalkyl” refers to an alkyl radical in which the alkyl group is substituted with one or more halogens. Typical haloalkyl groups include, but are not limited to, trifluoromethyl (—CF₃), difluoromethyl (—CHF₂), fluoromethyl (—CH₂F), chloromethyl (—CH₂Cl), dichloromethyl (—CHCl₂), tribromomethyl (—CH₂Br), and the like.

“Hydroxy” refers to the radical —OH.

“Nitro” refers to the radical —NO₂.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR═C(O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —S(═O)(═NR^{bb})R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂—NR^{bb}P(═O)(OR^{cc})₂—NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 Rad groups; or two geminal hydrogens on a carbon atom are replaced with the group ═O, ═S, ═NN(R^{bb})₂, —NNR^{bb}C(═O)R^{aa}, —NNR^{bb}C(═O)OR^{aa} ═NNR^{bb}S(═O)₂R^{aa}, ═NR^{bb}, or ═NOR^{cc};
each instance of R^{aa} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)R^{aa}, —C(═O)N(R^{cc}), —CO₂R^{aa}, —SO₂R′, —C(═NR^{cc})OR^{aa}, —C(═NR^{cc})N(R^{cc})₂, —SO₂N(R^{cc}), —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa}, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{f})₂, —N(R^{E})₂, —N(R)₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ee})₂, —OC(═O)N(R^{ee})₂, —NR^{ff}C(═O)R^{ee}, —NR^{ff}CO₂R^{ee}, —NRC(═O)N(R^{ee})₂, —C(═NR^{ff})OR^{ee}, —OC(═NR^{ff})R^{ee}, —OC(═NR^{ff})OR^{ee}, —C(═NR^{ff})N(R^{ee})₂, —OC(═NR^{ff})N(R^{ff})₂, —NR^{ff}C(═NR)N(e)₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee}), —OP(═O)(R^{ee}), —OP(═O)(OR^{ee})₂, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄⁻²sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc}), —CN, —C(═O)R^{aa}, —C(═O)N(R^{cc}), —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc}), —SO₂R^{aa}, —SO₂OR^{cc}, —SOR^{aa}, —C(═S)N(R^{cc}), —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and Claims. The invention is not intended to be limited in any manner by the above exemplary listing of substituents.

### Other Definitions

The term “about,” as used herein, includes the recited number ±10%. Thus, “about 10” means 9 to 11. As is understood by one skilled in the art, reference to “about” a value or parameter herein includes (and describes) instances that are directed to that value or parameter per se. For example, description referring to “about X” includes description of “X.”

“USP1” and “ubiquitin-specific-processing protease 1” as used herein refer to any native polypeptide or USP1-encoding polynucleotide. The term “USP1“encompasses” full-length,” unprocessed USP1 polypeptide as well as any forms of USP1 that result from processing within the cell (e g., removal of the signal peptide). The term also encompasses naturally occurring variants of USP1, e.g., those encoded by splice variants and allelic variants. The USP1 polypeptides described herein can be isolated from a variety of sources, such as from human tissue types or from another source, or prepared by recombinant or synthetic methods. Human USP1 sequences are known and include, for example, the sequences publicly available as UniProt No. 094782 (including isoforms). As used herein, the term “human USP1 protein” refers to USP1 protein comprising the amino acid sequence as set forth in SEQ ID NO: 1 in U.S. provisional patent application No. 62/857,986 filed Jun. 6, 2019.

USP1 is a deubiquitinating enzyme that acts as part of a complex with UAF1. USP1's “deubiquitinase activity” includes its ability to deubiquitinate as part of the USP1-UAF1 complex.

The term “specifically binds” to a protein or domain of a protein is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular protein or domain of a protein than it does with alternative proteins or domains. It should be understood that a molecule that specifically or preferentially binds to a first protein or domain may or may not specifically or preferentially bind to a second protein or domain. As such, “specific binding” or “preferential binding” does not necessarily require (although it can include) exclusive binding. Generally, but not necessarily, reference to binding means preferential binding. For example, a USP1 inhibitor that specifically binds to USP1, UAF1, and/or the USP1-UAF1 complex may not bind to other deubiquitinases, other USP proteins, or other UAF1 complexes (e.g., USP46-UAF1) or may bind to other deubiquitinases, other USP proteins, or other UAF1 complexes (e.g., USP46-UAF1) with a reduced affinity as compared to binding to USP1.

The terms “reduction” or “reduce” or “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

In some embodiments inhibiting USP1 proteins is the inhibition of one or more activities or functions of USP1 proteins. It should be appreciated that the activity or function of the one or more USP1 proteins may be inhibited in vitro or in vivo. Non limiting examples of activities and functions of USP1 include deubiquitinase activity, and formation of a complex with UAF 1 and are described herein. Exemplary levels of inhibition of the activity of one or more USP1 proteins include at least 10% inhibition, at least 20% inhibition, at least 30% inhibition, at least 40% inhibition, at least 50% inhibition, at least 60% inhibition, at least 70% inhibition, at least 80% inhibition, at least 90% inhibition, and up to 100% inhibition.

As used herein, the term “loss of function” mutation refers to a mutation that that results in the absence of a gene, decreased expression of a gene, or the production of a gene product (e.g., protein) having decreased activity or no activity. Loss of function mutations include for example, missense mutations, nucleotide insertions, nucleotide deletions, and gene deletions. Loss of function mutations also include dominant negative mutations. Thus, cancer cells with a loss of function mutation in a gene encoding BRCA1 include cancer cells that contain missense mutations in a gene encoding BRCA1 as well as cancer cells that lack a gene encoding BRCA1.

As used herein, the term “salt” refers to any and all salts and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds disclosed herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N*(Ci-4alkyl)₄ salts.

Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomologus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In some embodiments, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

In general, the “effective amount” of a compound refers to an amount sufficient to elicit the desired biological response. As will be appreciated by those of ordinary skill in this art, the effective amount of a compound disclosed herein may vary depending on such factors as the desired biological endpoint, the pharmacokinetics of the compound, the disease being treated, the mode of administration, and the age, health, and condition of the subject. An effective amount encompasses therapeutic and prophylactic treatment. An effective amount encompasses therapeutic and prophylactic treatment (i.e., encompasses a “therapeutically effective amount” and a “prophylactically effective amount”).

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

The term “container” means any receptacle and closure therefore suitable for storing, shipping, dispensing, and/or handling a pharmaceutical product.

The term “insert” or “package insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

Compounds

As generally described herein, provided are compounds (e.g., compounds of Formula (I), (II) or a compound of Table 1 or pharmaceutically acceptable salts, hydrates, solvates, prodrugs, stereoisomers or tautomers thereof) that are ubiquitin-specific-processing protease 1 (USP1) inhibitors useful for treating diseases and disorders (e.g., cancers) associated with USP1.

Provided herein are compounds of Formula (I). Unless the context requires otherwise, reference throughout this specification to “a compound of Formula (I)” or “compounds of Formula (I)” refers to all embodiments of Formula (I), including, for example, compounds of (I), (II) as well as the compounds of Table 1.

In some embodiments, provided are compounds of Formula (I) or a pharmaceutically acceptable salt, hydrate, solvate, prodrug, stereoisomer, or tautomer thereof. In some embodiments, including any of the numbered embodiments described herein, the compounds are provided as free base or pharmaceutically acceptable salts. In some embodiments, including any of the numbered embodiments described herein, the compounds are provided as free base. In some embodiments, including any of the numbered embodiments described herein, the compounds are provided as pharmaceutically acceptable salts.

In some embodiments, provided is a compound of Formula (I) or a pharmaceutically acceptable salt, hydrate, solvate, prodrug, stereoisomer, or tautomer thereof; wherein:

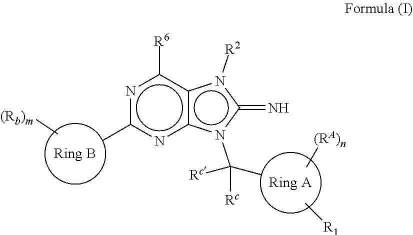

Ring B is a 5-6 member monocyclic aryl or heteroaryl;
Ring A is selected from C₆-C₁₀ aryl, 5-10 membered heteroaryl, —C₃-C₁₀ cycloalkyl, and 3-10 membered heterocyclyl;
R¹ is an optionally substituted 5-10 membered heteroaryl or an optionally substituted 3-10 membered heterocyclyl;
R² is selected from H, —C₁-C₆ alkyl, —C₁-C₆ haloalkyl, —C₁-C₆ heteroalkyl, —C₁-C₆ hydroxyalkyl, —C₃-C₁₀ cycloalkyl and arylalkyl, wherein each hydrogen of the alkyl, haloalkyl, heteroalkyl, hydroxylalkyl and arylalkyl can be independently replaced with a deuterium atom;
R⁶ is selected from H, -D, halo, —CN, —C₁-C₆ alkyl, —C₁-C₆ alkynyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, 6-10 member heteroaryl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, and —OC(═O)N(R^{a6})₂, wherein each alkyl, alkynyl, cycloalkyl, heterocyclyl, aryl, heteroaryl, arylalkyl and heteroarylalkyl is optionally substituted at any available position;
each R^{a6} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl;
each R^{A} is independently selected from -D, halo, —CN, —C₁-C₆ alkyl, —C₁-C₆ haloalkyl, —C₁-C₆ hydroxyalkyl, —C₃-C₁₀ cycloalkyl, —OR^{A1}, —N(R^{A1})₂; each R^{A1} is independently selected from H, —C₁-C₆ alkyl, —C₁-C₆ haloalkyl and C₃-C₉ cycloalkyl;
each R^{b} is independently selected from -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{b1}, —N(R^{b1})₂, —C(═O)R^{b1}, —C(═O)OR^{b1}, —NRIC(═O)R^{b1}, —NRC(═O)OR^{b}, —C(═O)N(R^{b1})₂, —OC(═O)N(R^{b1})₂, —S(═O)R^{b1}, —S(═O)₂R^{b1}, —SR¹, —S(═O)(═NR)R^{b1}, —NR^{b1}S(═O)₂R^{b1} and —S(═O)₂N(R^{b1})₂ or 2 R^{b} together with the atoms to which they are attached form a 4-7 member carbocyclyl or a 4-7 member heterocyclyl, wherein each alkyl, carbocylyl, cycloalkyl, heterocyclyl, aryl, heteroaryl, arylalkyl and heteroarylalkyl of R^{b} is optionally substituted at any available position;
each R^{b1} is independently selected from H, —C₁-C₆ alkyl (wherein each hydrogen can be independently replaced by deuterium), —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl;
each R^{c} and R^{c′} is independently selected from H, -D, —C₁-C₆ alkyl (e.g., -Me), —C₁-C₆ heteroalkyl and —C₁-C₆ haloalkyl or R^{c} and R^{c′} can be taken together with the atom to which they are attached to form a —C₃-C₉ cycloalkyl (e.g., cyclopropyl) or a carbonyl;
n is 0, 1, 2 or 3; and
m is 0, 1, 2 or 3.

In some embodiments, provided is a compound of Formula (I) or a pharmaceutically acceptable salt, hydrate, solvate, prodrug, stereoisomer, or tautomer thereof; wherein:

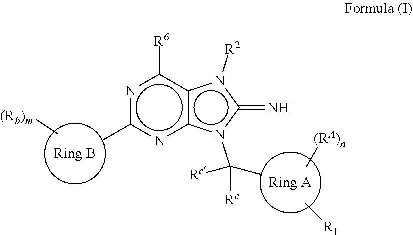

Ring B is a 5-6 member monocyclic aryl or heteroaryl;
Ring A is selected from C₆-C₁₀ aryl, 5-10 membered heteroaryl, —C₃-C₁₀ cycloalkyl, and 3-10 membered heterocyclyl;
R¹ is an optionally substituted 5-10 membered heteroaryl or an optionally substituted 3-10 membered heterocyclyl;
R² is selected from H, —C₁-C₆ alkyl, —C₁-C₆ haloalkyl, —C₁-C₆ heteroalkyl, —C₁-C₆ hydroxyalkyl, —C₃-C₁₀ cycloalkyl and arylalkyl, wherein each hydrogen of the alkyl, haloalkyl, heteroalkyl, hydroxylalkyl and arylalkyl can be independently replaced with a deuterium atom;
R⁶ is selected from H, -D, halo, —CN, —C₁-C₆ alkyl, —C₁-C₆ alkynyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, 6-10 member heteroaryl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, and —OC(═O)N(R^{a6})₂, wherein each alkyl, alkynyl, cycloalkyl, heterocyclyl, aryl, heteroaryl, arylalkyl and heteroarylalkyl is optionally substituted at any available position;
each R^{a6} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl;
each R^{A} is independently selected from -D, halo, —CN, —C₁-C₆ alkyl, —C₁-C₆ haloalkyl, —C₁-C₆ hydroxyalkyl, —C₃-C₁₀ cycloalkyl, —OR^{A1}, —N(R^{A1})₂;
each R^{A1} is independently selected from H, —C₁-C₆ alkyl, —C₁-C₆ haloalkyl and C₃-C₉ cycloalkyl;
each R^{b} is independently selected from -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{b1}, —N(R^{b1})₂, —C(═O)R^{b1}, —C(═O)OR^{b1}, -NRiC(═O)R^{b1}, —NRIC(═O)OR^{b1}, —C(═O)N(R^{b1})₂, —OC(═O)N(R^{b1})₂, —S(═O)R^{b1}, —S(═O)₂R^{b1}, —SR, —S(═O)(═NR^{b1})R^{b1}, —NR^{b1}S(═O)₂R^{b1} and —S(═O)₂N(R^{b1})₂ or 2 R^{b} together with the atoms to which they are attached form a 4-7 member carbocyclyl or a 4-7 member heterocyclyl, wherein each alkyl, carbocylyl, cycloalkyl, heterocyclyl, aryl, heteroaryl, arylalkyl and heteroarylalkyl of R^{b} is optionally substituted at any available position;
each R^{b1} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl;
each R^{c} and R^{c′} is independently selected from H, -D, —C₁-C₆ alkyl (e.g., -Me), —C₁-C₆ heteroalkyl and —C₁-C₆ haloalkyl or R^{c} and R^{c′} can be taken together with the atom to which they are attached to form a —C₃-C₉ cycloalkyl (e.g., cyclopropyl) or a carbonyl;
n is 0, 1, 2 or 3; and
m is 0, 1, 2 or 3.

In Formula (I), Formula (II) and the exemplary compounds and intermediates disclosed herein, the moiety

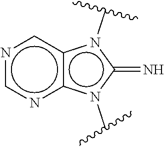

can alternatively and interchangeably be depicted as

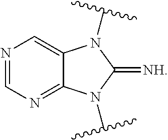

As generally defined herein, Ring B is a 5-6 member monocyclic aryl or heteroaryl. In some embodiments, Ring B is substituted with 0, 1, 2 or 3 instances of R^{b}. In some embodiments, Ring B is substituted with 0, 1 or 2 instances of R^{b}. In some embodiments, Ring B is substituted with 1 or 2 instances of R^{b}. In some embodiments, Ring B is substituted with 1 instance of R^{b}. In some embodiments, Ring B is substituted with 2 instances of R^{b}.

In certain embodiments, Ring B is a 5-membered heteroaryl containing 1-3 heteroatoms independently selected from O, N and S. In some embodiments, Ring B is a 5-membered heteroaryl containing 1-3 heteroatoms independently selected from O, N and S, substituted with 0, 1, 2 or 3 instances of R^{b}. In some embodiments, Ring B is a 5-membered heteroaryl ring selected from pyrrolyl, thiophenyl, furanyl, pyrazolyl, imidazolyl, thiazolyl, oxazolyl, isoxazolyl, isothiazolyl, triazolyl, tetrazolyl, oxadiazolyl and thiadiazolyl, each of which can be optionally substituted (e.g., substituted with 0, 1, 2 or 3 instances of R^{b}). In certain embodiments, ring B is selected from pyrazolyl, isoxazolyl and isothiazolyl. In some embodiments, Ring B is pyrazolyl (e.g., pyrazol-5-yl, pyrazol-4-yl). In other embodiments, Ring B is isoxazolyl (e.g., isoxazol-4-yl). In yet other embodiments, Ring B is isothiazolyl (e.g., isothiazol-4-yl).

In certain embodiments, Ring B is an optionally substituted 6 membered heteroaryl containing 1-3 nitrogen atoms. In certain embodiments, Ring B is a 6 membered heteroaryl containing 1-3 nitrogen atoms, substituted with 0, 1, 2 or 3 instances of R^{b}.

In some embodiments, Ring B is selected from pyridinyl, pyrimidinyl, pyrazinyl, triazinyl and pyridazinyl, each of which can be optionally substituted (e.g., substituted with 0, 1, 2 or 3 instances of R^{b}). In some embodiments, Ring B is selected from pyridinyl and pyrimidinyl, each of which can be optionally substituted (e.g., substituted with 0, 1, 2 or 3 instances of R).

In certain embodiments, Ring B is selected from phenyl, pyridinyl and pyrimidinyl, each of which can be optionally substituted (e.g., substituted with 0, 1, 2 or 3 instances of R). In some embodiments, Ring B is optionally substituted phenyl (e.g., substituted with 0, 1, 2 or 3 instances of R). In some embodiments, Ring B is optionally substituted pyridinyl (e.g., substituted with 0, 1, 2 or 3 instances of R). In some embodiments, Ring B is pyridin-1-yl, pyridin-2-yl or pyridin-3-yl, which can be optionally substituted (e.g., substituted with 0, 1, 2 or 3 instances of R^{b}). In other embodiments, Ring B is optionally substituted pyrimidinyl (e.g., substituted with 0, 1, 2 or 3 instances of R). In some embodiments, Ring B is pyrimidin-2-yl, pyrimidin-4-yl, pyrimidin-5-yl or pyrimidin-6-yl, which can be optionally substituted (e.g., substituted with 0, 1, 2 or 3 instances of R). In certain embodiments, Ring B is optionally substituted pyrimidin-5-yl (e.g., substituted with 0, 1, 2 or 3 instances of R^{b}).

As generally defined herein, each R^{b} is independently selected from -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{b1}, —N(R^{b1})₂, —C(═O)R^{b1}, —C(═O)OR^{b1}, —R^{b1}C(═O)R^{b}, —NR^{b1}C(═O)OR^{b1}, —C(═O)N(R^{b1})₂, —OC(═O)N(R^{b1})₂, —S(═O)R^{b1}, —S(═O)₂R^{b1}, —SR¹, —S(═O)(═NR¹)R^{b1}, —NR^{b1}S(═O)₂R^{b1} and —S(═O)₂N(R^{b1})₂ or 2 R^{b} together with the atoms to which they are attached form a 4-7 member carbocyclyl or a 4-7 member heterocyclyl, wherein each alkyl, carbocylyl, cycloalkyl, heterocyclyl, aryl, heteroaryl, arylalkyl and heteroarylalkyl of R^{b} is optionally substituted at any available position.

In certain embodiments, each R^{b} is independently selected from —CN, halo, —C₁-C₆ alkenyl, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, —OR^{b1} and —N(R^{b1})₂, or 2 R^{b} together with the atoms to which they are attached form a 4-7 member carbocyclyl or a 4-7 member heterocyclyl, wherein each aryl, alkyl, carbocyclyl, cycloalkyl and heterocyclyl is substituted with 0, 1, 2 or 3 instances of halo (e.g., —F, —Cl), —OH, —CN, -Me, -Et, —NH₂ or oxo and wherein each R^{b1} is independently selected from H, —C₁-C₆ alkyl, (wherein each hydrogen can be independently replaced by deuterium) —C₁-C₆ heteroalkyl, —C₁-C₆ haloalkyl and C₃-C₉ cycloalkyl.

In certain embodiments, each R^{b} is independently selected from halo (e.g., —Cl, —F), —CN, —C₁-C₆ alkenyl (e.g., vinyl, propenyl), —C₁-C₆ alkyl (e.g., -Me, -Et, —Pr, -ⁱPr, -ⁿBu, -sec-Bu, -iso-Bu, -^{t}Bu), —C₆-C₁₀ aryl (e.g., phenyl), —C₁-C₆ heteroalkyl (e.g., —CH₂NHCH2CH₃, —CH₂N(CH₃)CH₂CH₃, —CH₂N(CH₃)₂), —C₁-C₆ haloalkyl (e.g., —CF₃, —CIHF2, —CH₂CF₃), —C₁-C₆ hydroxyalkyl (e.g., —CH₂OH, —CH(OH)CF₃), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrrolidinyl, piperidinyl, piperazinyl, morpholinyl, 6-oxa-1-azaspiro[3.3]heptanyl, 6-oxa-1-azaspiro[3.4]octanyl), —OR^{b1} and —N(R^{b1})₂, or 2 R^{b} together with the atoms to which they are attached form a 4-7 member carbocyclyl or a 4-7 member heterocyclyl, wherein each aryl, alkyl, carbocyclyl, cycloalkyl and heterocyclyl is substituted with 0, 1, 2 or 3 instances of halo (e.g., —F, —Cl), and wherein each R^{b1} is independently selected from H, —C₁-C₆ alkyl (wherein each hydrogen can be independently replaced by deuterium) (e.g., -Me, -CD3, -Et, —Pr, -ⁱPr, -ⁿBu, -sec-Bu, -iso-Bu, -^{t}Bu), —C₁-C₆ haloalkyl (e.g., —CF₃, —CHF2, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R^{b} is independently selected from —CN, —C(═CH₂)CH₃, —C(CH₃)CH₂CH₃, —Cl, —F, -Me, -ⁱPr, —CH₂N(CH₃)CH₂CF₃, —CH₂N(CH₃)₂, —CH₂OH, —CH(OH)CF₃, —CF₃, —CH₂CF₃, cyclopropyl (substituted with 0, 1, or 2 instances of —F, -Me, —CN), azetidinyl (substituted with 0 or 1 instances of —F), phenyl (substituted with 0 or 1 instances of halo), —OCF₃, —OCH₂CF₃, —OCHF2, —OⁱPr, —OMe, —OCH₂CH(CH₃)₃, —N(Me)₂ and —NHMe, or 2 R^{b} together with the atoms to which they are attached form 1,3-dioxole substituted with 0, 1 or 2 instances of —F or -Me.

In certain embodiments, each R^{b} is independently selected from —CN, halo, —C₁-C₆ alkenyl, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, —OR^{b1} and —N(R^{b1})₂, or 2 R^{b} together with the atoms to which they are attached form a 4-7 member carbocyclyl or a 4-7 member heterocyclyl, wherein each aryl, alkyl, carbocyclyl, cycloalkyl and heterocyclyl is substituted with 0, 1, 2 or 3 instances of halo (e.g., —F, —Cl), —OH, —CN, -Me, -Et, —NH₂ or oxo and wherein each R^{b1} is independently selected from H, —C₁-C₆ alkyl, —C₁-C₆ heteroalkyl, —C₁-C₆ haloalkyl and C₃-C₉ cycloalkyl.

In certain embodiments, each R^{b} is independently selected from halo (e.g., —Cl, —F), —CN, —C₁-C₆ alkenyl (e.g., vinyl, propenyl), —C₁-C₆ alkyl (e.g., -Me, -Et, —Pr, -ⁱPr, -ⁿBu, -sec-Bu, -iso-Bu, -^{t}Bu), —C₆-C₁₀ aryl (e.g., phenyl), —C₁-C₆ heteroalkyl (e.g., —CH₂NHCH2CH₃, —CH₂N(CH₃)CH₂CH₃, —CH₂N(CH₃)₂), —C₁-C₆ haloalkyl (e.g., —CF₃, —CHF2, —CH₂CF₃), —C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrrolidinyl, piperidinyl, piperazinyl, morpholinyl, 6-oxa-1-azaspiro[3.3]heptanyl, 6-oxa-1-azaspiro[3.4]octanyl), —OR¹ and —N(R^{b1})₂, or 2 R^{b} together with the atoms to which they are attached form a 4-7 member carbocyclyl or a 4-7 member heterocyclyl, wherein each aryl, alkyl, carbocyclyl, cycloalkyl and heterocyclyl is substituted with 0, 1, 2 or 3 instances of halo (e.g., —F, —Cl), and wherein each R^{b1} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF2, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R^{b} is independently selected from —CN, —C(═CH₂)CH₃, —C(CH₃)CH₂CH₃, —Cl, —F, -Me, -ⁱPr, —CH₂N(CH₃)CH₂CF₃, —CF₃, —CH₂CF₃, cyclopropyl (substituted with 0 or 1 instance of —CN), azetidinyl (substituted with 0 or 1 instances of —F), phenyl (substituted with 0 or 1 instances of halo), —OCF₃, —OCH₂CF₃, —OCHF₂, —OⁱPr, —OMe, —OCH₂CH(CH₃)₃, —N(Me)₂ and —NHMe, or 2 R^{b} together with the atoms to which they are attached form 1,3-dioxole substituted with 0, 1 or 2 instances of —F or -Me.

In some embodiments, each R^{b} is independently selected from —CN, —C(═CH₂)CH₃, —F, -ⁱPr, —CF₃, cyclopropyl (substituted with 0 or 1 instance of —CN), —OCF₃, —OCHF₂, and —OMe.

In some embodiments R^{b} is -D.

In certain embodiments, R^{b} is halo (e.g., fluoro, chloro, bromo, iodo). In some embodiments, R^{b} is —Cl. In some embodiments, R^{b} is —F. In some embodiments, R^{b} is —Br. In some embodiments, R^{b} is —I.

In some embodiments, R^{b} is —CN.

In certain embodiments, R^{b} is —C₁-C₆ alkyl (e.g., -Me, -Et, —Pr, -ⁱPr, -ⁿBu, -sec-Bu, -iso-Bu, -^{t}Bu). In some embodiments, R^{b} is -Me. In some embodiments, R^{b} is -Et. In some embodiments R^{b} is —Pr. In some embodiments, R^{b} is -iPr.

In certain embodiments, R^{b} is —C₁-C₆ alkenyl (e.g., vinyl, propenyl). In some embodiments, R^{b} is vinyl. In some embodiments, R^{b} is propenyl (e.g., prop-1-en-1-yl, prop-1-en-2-yl). In some embodiments, R^{b} is prop-1-en-2-yl (—C(═CH₂)CH₃).

In some embodiments, R^{b} is —C₁-C₆ heteroalkyl. In some embodiments, R^{b} is methoxymethyl (—CH₂OCH₃). In some embodiments, R^{b} is aminomethyl (e.g., —CH₂NHCH₂CH₃, —CH₂N(CH₃)CH₂CF₃, —CH₂N(CH₃)CH₂CH₃, —CH₂N(CH₃)₂—CH₂NH₂, —CH₂NHCH₃, —CH₂N(CH₃)₂). In some embodiments, R^{b} is —CH₂N(CH₃)CH₂CF₃.

In some embodiments, R^{b} is —C₁-C₆ haloalkyl. In some embodiments, R^{b} is trifluoromethyl (—CF₃). In other embodiments, R^{b} is difluoromethyl (—CHF₂).

In some embodiments, R^{b} is —C₁-C₆ hydroxyalkyl (e.g., —CH₂OH, —CH₂CH₂OH, —CH(OH)CF₃). In some embodiments, R^{b} is hydroxymethyl (—CH₂OH).

In some embodiments, R^{b} is —C₃-C₁₀ cycloalkyl (e.g., cyclopropyl, cyclobutyl, cyclopentyl, cyclohexyl), each of which can be optionally substituted. In some embodiments, R^{b} is optionally substituted cyclopropy (e.g., cyclopropyl substituted with 0, 1 or 2 instances of —F, -Me or —CN or cyclopropyl substituted with 0, 1 or 2 instances of —F, -Me or —CN). In some embodiments R^{b} is cyclobutyl. In some embodiments, R^{b} is cyclopentyl. In some embodiments, R^{b} is cyclohexyl.

In some embodiments, R^{b} is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl, 6-oxa-1-azaspiro[3.3]heptanyl, 6-oxa-1-azaspiro[3.4]octanyl), each of which can be optionally substituted. In some embodiments, R^{b} is oxetanyl. In some embodiments, R^{b} is tetrahydropyranyl. In some embodiments, R^{b} is tetrahydrofuranyl. In some embodiments, R^{b} is azetidinyl (e.g., azetidinyl substituted with 0 or 1 instances of halo or methyl). In some embodiments, R^{b} is pyrrolidinyl. In some embodiments, R^{b} is piperidinyl. In some embodiments, R^{b} is piperazinyl. In some embodiments, R^{b} is morpholinyl. In some embodiments, R^{b} is azepanyl. In some embodiments, R^{b} is 6-oxa-1-azaspiro[3.3]heptanyl. In some embodiments, R^{b} is 6-oxa-1-azaspiro[3.4]octanyl.

In some embodiments, R^{b} is optionally substituted —C₆-C₁₀ aryl (e.g., phenyl, naphthyl). In some embodiments, R^{b} is optionally substituted phenyl (e.g., phenyl substituted with 0 or 1 instances of halo (e.g., —Cl, —F)).

In some embodiments R^{b} is cycloalkylalkyl (e.g., cyclopropylmethyl, cyclobutylmethyl, cyclopentylmethyl, cyclohexylmethyl, cycloheptylmethyl). In some embodiments, R^{b} is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R^{b} is arylalkyl. In some embodiments, R^{b} is benzyl.

In some embodiments, R^{b} is heteroarylalkyl (e.g., pyridinylmethyl, thiazolylmethyl, triazolylmethyl, pyrazolylmethyl).

In some embodiments, R^{b} is —OR¹ (e.g., hydroxy (—OH), methoxy, -OCD₃, difluoromethoxy (—OCHF₂), trifluoromethoxy (—OCF₃), —OCH(CH₃)CF₃, —OCH₂CF₃, ethoxy, propoxy, isopropoxy, —OCH₂CH(CH₃)₃, cyclopropyloxy, cyclobutyloxy). In some embodiments, R^{b} is hydroxy. In some embodiments, R^{b} is methoxy. In some embodiments, R^{b} is ethoxy. In some embodiments, R^{b} is propoxy. In some embodiments, R^{b} is isopropoxy. In some embodiments R^{b} is difluoromethoxy (—OCHF₂). In some embodiments, R^{b} is trifluoromethoxy (—OCF₃). In some embodiments, R^{b} is —OCH(CH₃)CF₃. In some embodiments, R^{b} is —OCH₂CF₃. In some embodiments, R^{b} is cyclopropyloxy.

In some embodiments, R^{b} is —N(R^{b1})₂ (e.g., —NH₂, —NHR^{b1}, —N(CH₃)R^{b1}). In some embodiments, R^{b} is —NH₂. In some embodiments, R^{b} is —NHR^{b1} (e.g., —NHMe, -NHEt, —NHPr, —NHⁱPr, -NHcyclopropyl, -NHcyclobutyl). In some embodiments, R^{b} is —N(CH₃)R^{b1} (e.g., —NMe₂, —N(CH₃)Et, —N(CH₃)Pr, —N(CH₃)ⁱPr, —N(CH₃)cyclopropyl, —N(CH₃)cyclobutyl).

In some embodiments, R^{b} is —C(═O)R^{b1} or —C(═O)OR^{b1}. In some embodiments, R^{b} is —C(═O)R^{b1} wherein R^{b1} is as described herein. In some embodiments, R^{b} is —C(═O)alkyl. In some embodiments, R^{b} is —C(O)CH₃, —C(O)cyclopropyl, —C(O)cyclobutyl, —C(O)^{t}Bu, —C(O)ⁱPr, —C(O)Pr, —C(O)^{t}Bu, or —C(═O)OMe. In some embodiments, R^{b} is acetyl (—C(═O)Me). In some embodiments, R^{b} is —C(═O)OR^{b1}. In some embodiments, R^{b} is —COOH. In some embodiments, R^{b} is COOMe.

In some embodiments, R^{b} is —NR^{b1}C(═O)R^{b1}. In certain embodiments, R^{b} is —NHC(═O)R^{b1} (e.g., —NH, —NHC(═O)Me, —NH, —NHC(═O)Et, —NH, —NHC(═O)Pr, —NH, —NHC(═O)ⁱPr, —NH, —NHC(═O)Bu, —NH, —NHC(═O)^{t}Bu, —NH, —NHC(═O)Cyclopropyl, —NH, —NHC(═O)Cyclobutyl). In some embodiments, R^{b} is —N(CH₃)C(═O)R^{b1} (e.g., —N(CH₃)C(═O)Me, —N(CH₃)C(═O)Et, —N(CH₃)C(═O)Pr, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R^{b} is —NR^{b1}C(═O)OR^{b1}. In certain embodiments, R^{b} is —NHC(═O)OR^{b1} (e.g., —NH, —NHC(═O)OMe, —NH, —NHC(═O)OEt, —NH, —NHC(═O)OPr, —NH, —NHC(═O)OⁱPr, —NH, —NHC(═O)OBu, —NH, —NHC(═O)O^{t}Bu, —NH, —NHC(═O)OCyclopropyl, —NH, —NHC(═O)OCyclobutyl). In some embodiments, R^{b} is —N(CH₃)C(═O)OR^{b1} (e.g., —N(CH₃)C(═O)OMe, —N(CH₃)C(═O)OEt, —N(CH₃)C(═O)OPr, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R^{b} is —C(═O)N(R^{b1})₂ (e.g., —C(═O)NH₂, —C(═O)NHR^{b1}, —C(═O)N(CH₃)R^{b1}). In some embodiments, R^{b} is —C(═O)NH₂. In certain embodiments, R^{b} is —C(═O)NHR^{b1} (e.g., —C(═O)NHMe, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R^{b} is —C(═O)N(CH₃)R^{b1} (e.g., —C(═O)NMe₂, —C(═O)N(CH₃)Et, —C(═O)N(CH₃)Pr, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R^{b} is —OC(═O)N(R^{b1})₂. In certain embodiments, R^{b} is —OC(═O)NHR (e.g., —OC(═O)NHMe, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R^{b} is —OC(═O)N(CH₃)R^{b1} (e.g., —OC(═O)NMe2, —OC(═O)N(CH₃)Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R^{b} is —S(═O)R^{b1}. In certain embodiments, R^{b} is —S(═O)alkyl (e.g., —S(═O)Me, —S(═O)Et, —S(═O)Pr, —S(═O)ⁱPr). In certain embodiments, R^{b} is —S(═O)cycloalkyl (e.g., —S(═O)cyclopropyl, —S(═O)cyclobutyl, —S(═O)cyclopentyl, —S(═O)cyclohexyl).

In some embodiments, R^{b} is —S(═O)₂R^{b1}. In certain embodiments, R^{b} is —S(═O)₂alkyl (e.g., —S(═O)₂Me, —S(═O)₂Et, —S(═O)₂Pr, —S(═O)₂′Pr). In certain embodiments, R^{b} is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R^{b} is S(═O)₂aryl (e.g., S(═O)₂phenyl).

In some embodiments, R^{b} is —SR^{b1}. In certain embodiments, R^{b} is -Salkyl (e.g., —SMe, -SEt, —SPr, —SⁱPr). In certain embodiments, R^{b} is -Scycloalkyl (e.g., -Scyclopropyl, -Scyclobutyl, -Scyclopentyl, -Scyclohexyl). In certain embodiments, R^{b} is -Saryl (e.g., Sphenyl).

In some embodiments, R^{b} is —S(═O)(═NR^{b1})R^{b1}. In certain embodiments, R^{b} is —S(═O)(═NH)R^{b1} (e.g., —S(═O)(═NH)Me, —S(═O)(═NH)Et, —S(═O)(═NH)Pr, —S(═O)(═NH) Pr, —S(═O)(═NH)Bu, —S(═O)(═NH)^{t}Bu, —S(═O)(═NH)Cyclopropyl, —S(═O)(═NH)Cyclobutyl). In some embodiments, R^{b} is —S(═O)(═NCH₃)R^{b1} (e.g., —S(═O)(═NCH₃)Me, —S(═O)(═NCH₃)Et, —S(═O)(═NCH₃)Pr, —S(═O)(═NCH₃) Pr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R^{b} is —R^{b1}S(═O)₂R^{b1}. In certain embodiments, R^{b} is —NHS(═O)₂alkyl (e.g., —NHS(═O)₂Me, —NHS(═O)₂Et, —NHS(═O)₂Pr, —NHS(═O)₂′Pr). In certain embodiments, R^{b} is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R^{b} is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂′Pr). In certain embodiments, R^{b} is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R^{b} is —S(═O)₂N(R^{b1})₂. (e.g., —S(═O)₂NH₂, —S(═O)₂NHR^{b1}, —S(═O)₂N(CH₃)R^{b1}). In some embodiments, R^{b} is —S(═O)₂NH₂. In some embodiments, R^{b} is —S(═O)₂NHR^{b1} (e.g., —S(═O)₂NHMe, —S(═O)₂NHEt, —S(═O)₂NHPr, —S(═O)₂NH′Pr, —S(═O)₂NHcyclopropyl, —S(═O)₂NHcyclobutyl). In some embodiments, R^{b} is —S(═O)₂N(CH₃)R^{b1} (e.g., —S(═O)₂NMe₂, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

In some embodiments, 2 R^{b} together with the atoms to which they are attached form a 4-7 member optionally substituted carbocyclyl or a 4-7 member optionally substituted heterocyclyl.

In some instances, the carbocyclyl or heterocyclyl are substituted with 0, 1, 2 or 3 instances of halo (e.g., —F, —Cl), —OH, —CN, -Me, -Et, —NH₂. In some instances, the ring formed by the 2 R^{b} groups is optionally substituted 1,3 dioxole (e.g., dioxole substituted with 0, 1 or 2 instances of —F or -Me).

As generally defined herein, each R^{b1} is independently selected from H, —C₁-C₆ alkyl (wherein each hydrogen can be independently replaced by deuterium), —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In some embodiments, each R^{b1} is independently selected from H, —C₁-C₆ alkyl (wherein each hydrogen can be independently replaced by deuterium) (e.g., -Me, -Et, —Pr, -ⁱPr, -ⁿBu, -^{t}Bu, -sec-Bu, -iso-Bu), —C₁-C₆ heteroalkyl (e.g., —CH₂OMe), —C₁-C₆ haloalkyl (e.g., —CHF₂, —CF₃, —CH(CH₃)CF₃, —CH₂CF₃) and C₃-C₉ cycloalkyl (e.g., cyclopropyl, cyclobutyl, cyclopentyl, cyclohexyl, cycloheptyl). In some embodiments, R^{b1} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R^{b1} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In some embodiments, each R^{b1} is independently selected from H, —C₁-C₆ alkyl (e.g., -Me, -Et, —Pr, -ⁱPr, -ⁿBu, -^{t}Bu, -sec-Bu, -iso-Bu), —C₁-C₆ heteroalkyl (e.g. —CH₂OMe), —C₁-C₆ haloalkyl (e.g., —CHF₂, —CF₃, —CH(CH₃)CF₃, —CH₂CF₃) and C₃-C₉ cycloalkyl (e.g., cyclopropyl, cyclobutyl, cyclopentyl, cyclohexyl, cycloheptyl). In some embodiments, R^{b1} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF2, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R^{b1} is independently H.

In some embodiments, each R^{b1} is independently —C₁-C₆ alkyl (e.g., -Me, -Et, —Pr, -ⁱPr, -ⁿBu, -^{t}Bu, -sec-Bu, -iso-Bu). In some embodiments, each R^{b1} is independently -Me. In some embodiments, each R^{b1} is independently -Et. In some embodiments, each R^{b1} is independently —Pr. In some embodiments, each R^{b1} is independently -ⁱPr.

In some embodiments, each R^{b1} is independently H.

In some embodiments, each R^{b1} is independently —C₁-C₆ alkyl (wherein each hydrogen can be independently replaced by deuterium) (e.g., -Me, -CD₃, -Et, —Pr, -ⁱPr, -ⁿBu, -^{t}Bu, -sec-Bu, -iso-Bu). In some embodiments, each R^{b1} is independently -Me. In some embodiments each R^{b1} is independently -CD₃. In some embodiments, each R^{b1} is independently -Et. In some embodiments, each R^{b1} is independently —Pr. In some embodiments, each R^{b1} is independently -ⁱPr.

In some embodiments, each R^{b1} is independently —C₁-C₆ heteroalkyl. In some embodiments, each R^{b1} is independently methoxymethyl (—CH₂OCH₃). In some embodiments, each R^{b1} is independently aminomethyl (e.g., —CH₂NH₂, —CH₂NHCH₃, —CH₂N(CH₃)₂.

In some embodiments, each R^{b1} is independently —C₁-C₆ haloalkyl. In some embodiments, each R^{b1} is independently trifluoromethyl (—CF₃). In other embodiments, each R^{b1} is independently difluoromethyl (—CHF₂). In some embodiments, R^{b1} is —CH₂F. In some embodiments, each R^{b1} is —CH(CH₃)CF₃. In some embodiments, each R^{b1} is —CH₂CF₃.

In some embodiments, each R^{b1} is independently —C₃-C₉ cycloalkyl (e.g., cyclopropyl, cyclobutyl, cyclopentyl, cyclohexyl). In some embodiments, each R^{b1} is independently cyclopropyl. In some embodiments each R^{b1} is independently cyclobutyl. In some embodiments, each R^{b1} is independently cyclopentyl. In some embodiments, each R^{b1} is independently cyclohexyl.

In some embodiments, each R^{b1} is independently 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl).

In some embodiments, R^{b1} is independently heteroaryl. In some embodiments, R^{b1} is independently a 5-10 member heteroaryl (e.g., a 5-6 member monocyclic heteroaryl or an 8-10 member bicyclic heteroaryl containing 1-3 heteroatoms independently selected from N, O and S).

In some embodiments, R^{b1} is independently a 5-6 member monocyclic heteroaryl (e.g., a 5-member monocyclic heteroaryl containing 1-3 heteroatoms independently selected from O, N and S, a 6-member monocyclic heteroaryl containing 1-3 N heteroatoms). In some embodiments, R^{b1} is independently a 5-member monocyclic heteroaryl (e.g., pyrazolyl, pyrolyl, thiophenyl, furyl, thiazolyl, isothiazolyl, oxazolyl, isoxazolyl, imidazolyl, triazolyl, thiadiazolyl, oxadiazolyl). In some embodiments, R^{b1} is independently thiophenyl (e.g., thiophen-2-yl, thiophen-3-yl). In some embodiments, R^{b1} is independently pyrazolyl (e.g., pyrazol-1-yl, pyrazol-3-yl, pyrazol-5-yl). In some embodiments, R^{b1} is independently thiazolyl (e.g., thiazol-2-yl, thiazol-4-yl, thiazol-5-yl). In some embodiments, R^{b1} is independently a 6-member monocyclic heteroaryl (e.g., pyridyl, pyrimidinyl, triazinyl, pyrazinyl, pyridazinyl). In some embodiments, R^{b1} is independently pyridinyl (e.g., pyridin-2-yl, pyridin-3-yl, pyridin-4-yl). In some embodiments, R^{b1} is independently pyrimidinyl (e.g., pyrimidin-2-yl, pyrimidin-4-yl, pyrimidin-5-yl).

In some embodiments, R^{b1} is independently aryl. In some embodiments, R^{b1} is independently 6-10 member mono or bicyclic aryl. In some embodiments, R^{b1} is independently phenyl.

In some embodiments each R^{b1} is independently cycloalkylalkyl (e.g., cyclopropylmethyl, cyclobutylmethyl, cyclopentylmethyl, cyclohexylmethyl, cycloheptylmethyl). In some embodiments, each R^{b1} is independently heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, each R^{b1} is independently arylalkyl. In some embodiments, each R^{b1} is independently benzyl.

In some embodiments, each R^{b1} is independently heteroarylalkyl (e.g., pyridinylmethyl, thiazolylmethyl, triazolylmethyl, pyrazolylmethyl).

In some embodiments, provided is a compound of Formula (II) or a pharmaceutically acceptable salt, hydrate, solvate, prodrug, stereoisomer, or tautomer thereof; wherein:

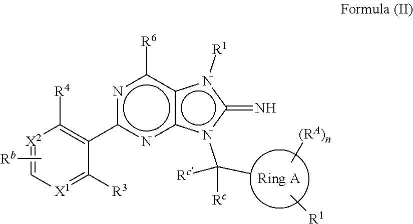

X¹ is selected from CH and N;
X² is selected from CH and N;
R³ is selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂ wherein each alkyl, cycloalkyl, heterocyclyl, aryl, heteroaryl, arylalkyl and heteroarylalkyl is optionally substituted at any available position;
R⁴ is selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂ wherein each alkyl, cycloalkyl, heterocyclyl, aryl, heteroaryl, arylalkyl and heteroarylalkyl is optionally substituted at any available position; and
each R^{a3} and R^{a4} is independently selected from H, —C₁-C₆ alkyl (wherein each hydrogen can be replaced by deuterium), —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In some embodiments, provided is a compound of Formula (II) or a pharmaceutically acceptable salt, hydrate, solvate, prodrug, stereoisomer, or tautomer thereof; wherein:

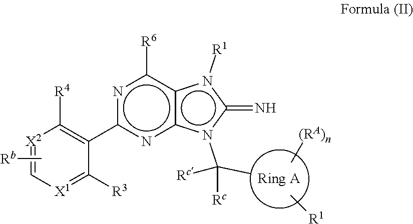

X¹ is selected from CH and N;
X² is selected from CH and N;
R³ is selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂ wherein each alkyl, cycloalkyl, heterocyclyl, aryl, heteroaryl, arylalkyl and heteroarylalkyl is optionally substituted at any available position;
R⁴ is selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂ wherein
each alkyl, cycloalkyl, heterocyclyl, aryl, heteroaryl, arylalkyl and heteroarylalkyl is optionally substituted at any available position; and each R^{a3} and R^{a4} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In certain embodiments, X¹ is CH. In other embodiments, X¹ is N.

In certain embodiments, X² is CH. In other embodiments, X² is N. In some embodiments X¹ is N and X² is CH. In some embodiments, X¹ is CH and X² is CH. In some embodiments X¹ is N and X² is N. In some embodiments X¹ is CH and X² is N.

In some embodiments, the moiety represented by

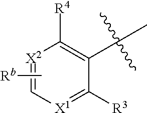

is selected from:

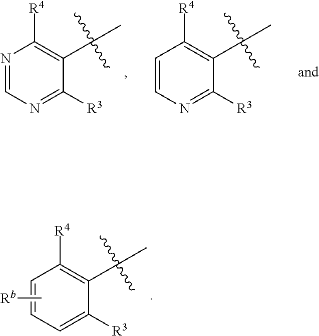

As generally defined herein, each R³ is independently selected from H, -D, halo, —CN, —C₁-C₆ alkyl (wherein each hydrogen can be replaced by deuterium), —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂ wherein each alkyl, cycloalkyl, heterocyclyl, aryl, heteroaryl, arylalkyl and heteroarylalkyl is optionally substituted at any available position.

In some embodiments, each R³ is independently selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, —OR^{a3} and —N(R^{a3})₂, wherein each aryl, alkyl, cycloalkyl and heterocyclyl is substituted with 0, 1, 2 or 3 instances of halo (e.g., —F, —Cl), —OH, —CN, -Me, -Et, —NH₂ or oxo and wherein each R^{a3} is independently selected from H, —C₁-C₆ alkyl (wherein each hydrogen can be replaced by deuterium), —C₁-C₆ heteroalkyl, —C₁-C₆ haloalkyl and C₃-C₉ cycloalkyl.

In some embodiments, each R³ is independently selected from H, -D, halo (e.g., —F, —Cl), —CN, —C₁-C₆ alkyl (e.g., -Me, -Et, —Pr, -ⁱPr, -ⁿBu, -sec-Bu, -iso-Bu, -^{t}Bu), —C₁-C₆ alkenyl (e.g., vinyl, propenyl), —C₁-C₆ heteroalkyl (e.g., —CH₂NHCH2CH₃, —CH₂N(CH₃)CH₂CH₃, —CH₂N(CH₃)₂), —C₁-C₆ haloalkyl (e.g., —CF₃, —CHF2, —CH₂CF₃), —C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrrolidinyl, piperidinyl, piperazinyl, morpholinyl, 6-oxa-1-azaspiro[3.3]heptanyl, 6-oxa-1-azaspiro[3.4]octanyl), —C₆-C₁₀ aryl (e.g., phenyl), —OR^{a3} and —N(R^{a3})₂, wherein each aryl, alkyl, cycloalkyl and heterocyclyl is substituted with 0, 1, 2 or 3 instances of halo (e.g., —F, —Cl), and wherein each R^{a3} is independently selected from H, —C₁-C₆ alkyl (wherein each hydrogen can be replaced by deuterium) (e.g., -Me, -Et, —Pr, -ⁱPr, -ⁿBu, -sec-Bu, -iso-Bu, -^{t}Bu), —C₁-C₆ haloalkyl (e.g., —CF₃, —CHF2, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R³ is independently selected from H, -D, —CN, —C(═CH₂)CH₃, —C(CH₃)CH₂CH₃, —Cl, —F, -Me, -ⁱPr, —CH₂N(CH₃)CH₂CF₃, —CF₃, —CH₂CF₃, cyclopropyl (substituted with 0 or 1 instance of —CN), azetidinyl (substituted with 0 or 1 instances of —F), phenyl (substituted with 0 or 1 instances of halo), —OCF₃, —OCH₂CF₃, —OCHF2, —OCH₂F, —OⁱPr, —OMe, -OEt, -OCD3, —OCH₂CH(CH₃)₃, —N(Me)₂ and —NHMe and —NHⁱPr.

In some embodiments, each R³ is independently selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, -NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂ wherein each alkyl, cycloalkyl, heterocyclyl, aryl, heteroaryl, arylalkyl and heteroarylalkyl is optionally substituted at any available position.

In some embodiments, each R³ is independently selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, —OR^{a3} and —N(R^{a3})₂, wherein each aryl, alkyl, cycloalkyl and heterocyclyl is substituted with 0, 1, 2 or 3 instances of halo (e.g., —F, —Cl), —OH, —CN, -Me, -Et, —NH₂ or oxo and wherein each R^{a3} is independently selected from H, —C₁-C₆ alkyl, —C₁-C₆ heteroalkyl, —C₁-C₆ haloalkyl and C₃-C₉ cycloalkyl.

In some embodiments, each R³ is independently selected from H, -D, halo (e.g., —F, —Cl), —CN, —C₁-C₆ alkyl (e.g., -Me, -Et, —Pr, -ⁱPr, -ⁿBu, -sec-Bu, -iso-Bu, -^{t}Bu), —C₁-C₆ alkenyl (e.g., vinyl, propenyl), —C₁-C₆ heteroalkyl (e.g., —CH₂NHCH2CH₃, —CH₂N(CH₃)CH₂CH₃, —CH₂N(CH₃)₂), —C₁-C₆ haloalkyl (e.g., —CF₃, —CHF2, —CH₂CF₃), —C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrrolidinyl, piperidinyl, piperazinyl, morpholinyl, 6-oxa-1-azaspiro[3.3]heptanyl, 6-oxa-1-azaspiro[3.4]octanyl), —C₆-C₁₀ aryl (e.g., phenyl), —OR^{a3} and —N(R^{a3})₂, wherein each aryl, alkyl, cycloalkyl and heterocyclyl is substituted with 0, 1, 2 or 3 instances of halo (e.g., —F, —Cl), and wherein each R^{a3} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF2, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R³ is independently selected from H, -D, —CN, —C(═CH₂)CH₃, —C(CH₃)CH₂CH₃, —Cl, —F, -Me, -ⁱPr, —CH₂N(CH₃)CH₂CF₃, —CF₃, —CH₂CF₃, cyclopropyl (substituted with 0 or 1 instance of —CN), azetidinyl (substituted with 0 or 1 instances of —F), phenyl (substituted with 0 or 1 instances of halo), —OCF₃, —OCH₂CF₃, —OCHF2, —OⁱPr, —OMe, —OCH₂CH(CH₃)₃, —N(Me)₂ and —NHMe and —NHⁱPr.

In some embodiments, R³ is H. In some embodiments R³ is -D.

In certain embodiments, R³ is halo (e.g., fluoro, chloro, bromo, iodo). In some embodiments, R³ is —C₁. In some embodiments, R³ is —F. In some embodiments, R³ is —Br. In some embodiments, R³ is —I.

In some embodiments, R³ is —CN.

In certain embodiments, R³ is —C₁-C₆ alkyl (e.g., -Me, -Et, —Pr, -ⁱPr, -ⁿBu, -sec-Bu, -iso-Bu, -^{t}Bu, —C(CH₃)CH₂CH₃). In some embodiments, R³ is -Me. In some embodiments, R³ is -Et. In some embodiments R³ is —Pr. In some embodiments, R³ is -iPr. In some embodiments, R³ is —C(CH₃)CH₂CH₃.

In certain embodiments, R³ is —C₁-C₆ alkenyl (e.g., vinyl, propenyl). In some embodiments, R³ is vinyl. In some embodiments, R³ is propenyl (e.g., prop-1-en-1-yl, prop-1-en-2-yl). In some embodiments, R³ is prop-1-en-2-yl (—C(═CH₂)CH₃).

In some embodiments, R³ is —C₁-C₆ heteroalkyl. In some embodiments, R³ is methoxymethyl (—CH₂OCH₃). In some embodiments, R³ is aminomethyl (e.g., —CH₂NH₂, —CH₂NHCH3, —CH₂NHCH2CH₃, —CH₂N(CH₃)₂). In some embodiments, R³ is —CH₂N(CH₃)CH₂CH₃. In some embodiments, R³ is —CH₂N(CH₃)CH₂CF₃.

In some embodiments, R³ is —C₁-C₆ haloalkyl (e.g., —CF₃, —CHF2, —CH₂CF₃). In some embodiments, R³ is trifluoromethyl (—CF₃). In other embodiments, R³ is difluoromethyl (—CHF2).

In other embodiments, R³ is —CH₂CF₃.

In some embodiments, R³ is —C₁-C₆ hydroxyalkyl (e.g., —CH₂OH, —CH₂CH₂OH), —CH(OH)CF₃). In some embodiments, R³ is hydroxymethyl (—CH₂OH).

In some embodiments, R³ is optionally substituted —C₃-C₁₀ cycloalkyl (e.g., cyclopropyl, cyclobutyl, cyclopentyl, cyclohexyl). In some embodiments, R³ is optionally substituted cyclopropyl (e.g., substituted with 0 or 1 instance of —CN). In some embodiments R³ is cyclobutyl. In some embodiments, R³ is cyclopentyl. In some embodiments, R³ is cyclohexyl.

In some embodiments, R³ is an optionally substituted 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl, 6-oxa-1-azaspiro[3.3]heptanyl, 6-oxa-1-azaspiro[3.4]octanyl). In some embodiments, R³ is oxetanyl. In some embodiments, R³ is tetrahydropyranyl. In some embodiments, R³ is tetrahydrofuranyl. In some embodiments, R³ is azetidinyl. In certain embodiments, the azetidinyl is optionally substituted (e.g., substituted with 0 or 1 instances of —F or -Me). In some embodiments, R³ is pyrrolidinyl. In some embodiments, R³ is piperidinyl. In some embodiments, R³ is piperazinyl. In some embodiments, R³ is morpholinyl. In some embodiments, R³ is azepanyl. In some embodiments, R³ is 6-oxa-1-azaspiro[3.3]heptanyl. In some embodiments, R³ is 6-oxa-1-azaspiro[3.4]octanyl.

In some embodiments R³ is cycloalkylalkyl (e.g., cyclopropylmethyl, cyclobutylmethyl, cyclopentylmethyl, cyclohexylmethyl, cycloheptylmethyl). In some embodiments, R³ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R³ is arylalkyl. In some embodiments, R³ is benzyl.

In some embodiments, R³ is heteroarylalkyl (e.g., pyridinylmethyl, thiazolylmethyl, triazolylmethyl, pyrazolylmethyl).

In some embodiments, R³ is optionally subsituted —C₆-C₁₀ aryl (e.g., phenyl, naphthyl). In some embodiments, R³ is optionally substituted phenyl (e.g., phenyl substituted with 0 or 1 instances of halo (e.g., —Cl, —F)). In certain embodiments, R³ is -2-C₁-phenyl.

In some embodiments, R³ is —OR^{a3} (e.g., hydroxy (—OH), methoxy, -OCD3, difluoromethoxy (—OCHF2), fluoromethoxy (—OCH₂F), trifluoromethoxy (—OCF₃), —OCH(CH₃)CF₃, —OCH₂CF₃, ethoxy, propoxy, isopropoxy, cyclopropyloxy, cyclobutyloxy, —OCH₂CH(CH₃)₃). In some embodiments, R³ is hydroxy. In some embodiments, R³ is methoxy. In some embodiments, R³ is ethoxy. In some embodiments, R³ is propoxy. In some embodiments, R³ is isopropoxy. In some embodiments R³ is difluoromethoxy (—OCHF2). In some embodiments, R³ is trifluoromethoxy (—OCF₃). In some embodiments, R³ is —OCH(CH₃)CF₃. In some embodiments, R³ is —OCH₂CF₃. In some embodiments, R³ is cyclopropyloxy. In some embodiments R³ is —OCH₂CH(CH₃)₃.

In some embodiments, R³ is —N(R^{a3})₂ (e.g., —NH₂, —N—R^{a3}, —N(CH₃)R^{a3}). In some embodiments, R³ is —NH₂. In some embodiments, R³ is -NHR^{a3} (e.g., —NHMe, —NHEt, —NHPr, —NHⁱPr, -NHcyclopropyl, -NHcyclobutyl). In some embodiments, R³ is —N(CH₃)R^{a3} (e.g., —NMe₂, —N(CH₃)Et, —N(CH₃)Pr, —N(CH₃)ⁱPr, —N(CH₃)cyclopropyl, —N(CH₃)cyclobutyl).

In some embodiments, R³ is —C(═O)R^{a3} or —C(═O)OR^{a3}. In some embodiments, R³ is —C(═O)R^{a3} wherein R^{a3} is as described herein. In some embodiments, R³ is —C(═O)alkyl. In some embodiments, R³ is —C(O)CH₃, —C(O)cyclopropyl, —C(O)cyclobutyl, —C(O)^{t}Bu, —C(O)ⁱPr, —C(O)Pr, —C(O)^{t}Bu, or —C(═O)OMe. In some embodiments, R³ is acetyl (—C(═O)Me). In some embodiments, R³ is —C(═O)OR^{a3}. In some embodiments, R³ is —COOH. In some embodiments, R³ is COOMe.

In some embodiments, R³ is —NR^{a3}C(═O)R^{a3}. In certain embodiments, R³ is —NHC(═O)R^{a3} (e.g., —NH, —NHC(═O)Me, —NH, —NHC(═O)Et, —NH, —NHC(═O)Pr, —NH, —NHC(═O)ⁱPr, —NH, —NHC(═O)Bu, —NH, —NHC(═O)^{t}Bu, —NH, —NHC(═O)Cyclopropyl, —NH, —NHC(═O)Cyclobutyl). In some embodiments, R³ is —N(CH₃)C(═O)R^{a3} (e.g., —N(CH₃)C(═O)Me, —N(CH₃)C(═O)Et, —N(CH₃)C(═O)Pr, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R³ is —NR^{a3}C(═O)OR^{a3}. In certain embodiments, R³ is —NHC(═O)OR^{a3} (e.g., —NH, —NHC(═O)OMe, —NH, —NHC(═O)OEt, —NH, —NHC(═O)OPr, —NHC(═O)OⁱPr, —NHC(═O)OBu, —NHC(═O)O^{t}Bu, —NHC(═O)OCyclopropyl, —NHC(═O)OCyclobutyl). In some embodiments, R³ is —N(CH₃)C(═O)OR^{a3} (e.g., —N(CH₃)C(═O)OMe, —N(CH₃)C(═O)OEt, —N(CH₃)C(═O)OPr, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R³ is —C(═O)N(R^{a3})₂ (e.g., —C(═O)NH₂, —C(═O)NHR^{a3}, C(═O)N(CH₃)R^{a3}). In some embodiments, R³ is —C(═O)NH₂. In certain embodiments, R³ is —C(═O)NHR^{a3} (e.g., —C(═O)NHMe, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R³ is —C(═O)N(CH₃)R^{a3} (e.g., —C(═O)NMe₂, —C(═O)N(CH₃)Et, —C(═O)N(CH₃)Pr, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R³ is —OC(═O)N(R^{a3})₂. In certain embodiments, R³ is —OC(═O)NHR^{a3} (e.g., —OC(═O)NHMe, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R³ is —OC(═O)N(CH₃)R^{a3} (e.g., —OC(═O)NMe₂, —OC(═O)N(CH₃)Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R³ is —S(═O)R^{a3}. In certain embodiments, R³ is —S(═O)alkyl (e.g., —S(═O)Me, —S(═O)Et, —S(═O)Pr, —S(═O)ⁱPr). In certain embodiments, R³ is —S(═O)cycloalkyl (e.g., —S(═O)cyclopropyl, —S(═O)cyclobutyl, —S(═O)cyclopentyl, —S(═O)cyclohexyl).

In some embodiments, R³ is —S(═O)₂R^{a3}. In certain embodiments, R³ is —S(═O)₂alkyl (e.g., —S(═O)₂Me, —S(═O)₂Et, —S(═O)₂Pr, —S(═O)₂′Pr). In certain embodiments, R³ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R³ is S(═O)₂aryl (e.g., S(═O)₂phenyl).

In some embodiments, R³ is —SR^{a3}. In certain embodiments, R³ is -Salkyl (e.g., —SMe, -SEt, —SPr, —SⁱPr). In certain embodiments, R³ is -Scycloalkyl (e.g., -Scyclopropyl, -Scyclobutyl, -Scyclopentyl, -Scyclohexyl). In certain embodiments, R³ is -Saryl (e.g., Sphenyl).

In some embodiments, R³ is —S(═O)(═NR^{a3})R^{a3}. In certain embodiments, R³ is —S(═O)(═NH)R^{a3} (e.g., —S(═O)(═NH)Me, —S(═O)(═NH)Et, —S(═O)(═NH)Pr, —S(═O)(═NH) Pr, —S(═O)(═NH)Bu, —S(═O)(═NH)^{t}Bu, —S(═O)(═NH)Cyclopropyl, —S(═O)(═NH)Cyclobutyl). In some embodiments, R³ is —S(═O)(═NCH₃)R^{a3} (e.g., —S(═O)(═NCH₃)Me, —S(═O)(═NCH₃)Et, —S(═O)(═NCH₃)Pr, —S(═O)(═NCH₃) Pr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R³ is —NR^{a3}S(═O)₂R^{a3}. In certain embodiments, R³ is —NHS(═O)₂alkyl (e.g., —NHS(═O)₂Me, —NHS(═O)₂Et, —NHS(═O)₂Pr, —NHS(═O)₂′Pr). In certain embodiments, R³ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R³ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂′Pr). In certain embodiments, R³ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R³ is —S(═O)₂N(R^{a3})₂. (e.g., —S(═O)₂NH₂, —S(═O)₂NHR^{a3}, S(═O)₂N(CH₃)R^{a3}). In some embodiments, R³ is —S(═O)₂NH₂. In some embodiments, R³ is —S(═O)₂NHR^{a3} (e.g., —S(═O)₂NHMe, —S(═O)₂NHEt, —S(═O)₂NHPr, —S(═O)₂NH′Pr, —S(═O)₂NHcyclopropyl, —S(═O)₂NHcyclobutyl). In some embodiments, R³ is —S(═O)₂N(CH₃)R^{a3} (e.g., —S(═O)₂NMe2, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally defined herein, each R^{a3} is independently selected from H, —C₁-C₆ alkyl (wherein each hydrogen can be replaced by deuterium), —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In some embodiments, each R^{a3} is independently selected from H, —C₁-C₆ alkyl (wherein each hydrogen can be replaced by deuterium) (e.g., -Me, -CD₃, -Et, —Pr, -ⁱPr, -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R^{a3} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In some embodiments, each R^{a3} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R^{a3} is independently H.

In some embodiments, each R^{a3} is independently —C₁-C₆ alkyl (wherein each hydrogen can be replaced by deuterium) (e.g., -Me, -CD₃, -Et, —Pr, -ⁱPr, -ⁿBu, -^{t}Bu, -sec-Bu, -iso-Bu). In some embodiments, each R^{a3} is independently -Me. In some embodiments, each R^{a3} is independently -Et. In some embodiments, each R^{a3} is independently —Pr. In some embodiments, each R^{a3} is independently -ⁱPr.

In some embodiments, each R^{a3} is independently —C₁-C₆ heteroalkyl. In some embodiments, each R^{a3} is independently methoxymethyl (—CH₂OCH₃). In some embodiments, each R^{a3} is independently aminomethyl (e.g., —CH₂NH₂, —CH₂NHCH₃, —CH₂N(CH₃)₂.

In some embodiments, each R^{a3} is independently —C₁-C₆ haloalkyl. In some embodiments, each R^{a3} is independently trifluoromethyl (—CF₃). In other embodiments, each R^{a3} is independently difluoromethyl (—CHF₂). In some embodiments, each R^{a3} is independently —CH₂F. In some embodiments, each R^{a3} is —CH(CH₃)CF₃. In some embodiments, each R^{a3} is —CH₂CF₃.

In some embodiments, each R^{a3} is independently —C₃-C₉ cycloalkyl (e.g., cyclopropyl, cyclobutyl, cyclopentyl, cyclohexyl). In some embodiments, each R^{a3} is independently cyclopropyl. In some embodiments each R^{a3} is independently cyclobutyl. In some embodiments, each R^{a3} is independently cyclopentyl. In some embodiments, each R^{a3} is independently cyclohexyl.

In some embodiments, each R^{a3} is independently 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl).

In some embodiments, R^{a3} is independently heteroaryl. In some embodiments, R^{a3} is independently a 5-10 member heteroaryl (e.g., a 5-6 member monocyclic heteroaryl or an 8-10 member bicyclic heteroaryl containing 1-3 heteroatoms independently selected from N, O and S).

In some embodiments, R^{a3} is independently a 5-6 member monocyclic heteroaryl (e.g., a 5-member monocyclic heteroaryl containing 1-3 heteroatoms independently selected from O, N and S, a 6-member monocyclic heteroaryl containing 1-3 N heteroatoms). In some embodiments, R^{a3} is independently a 5-member monocyclic heteroaryl (e.g., pyrazolyl, pyrolyl, thiophenyl, furyl, thiazolyl, isothiazolyl, oxazolyl, isoxazolyl, imidazolyl, triazolyl, thiadiazolyl, oxadiazolyl). In some embodiments, R^{a3} is independently thiophenyl (e.g., thiophen-2-yl, thiophen-3-yl). In some embodiments, R^{a3} is independently pyrazolyl (e.g., pyrazol-1-yl, pyrazol-3-yl, pyrazol-5-yl). In some embodiments, R^{a3} is independently thiazolyl (e.g., thiazol-2-yl, thiazol-4-yl, thiazol-5-yl).

In some embodiments, R^{a3} is independently a 6-member monocyclic heteroaryl (e.g., pyridyl, pyrimidinyl, triazinyl, pyrazinyl, pyridazinyl). In some embodiments, R^{a3} is independently pyridinyl (e.g., pyridin-2-yl, pyridin-3-yl, pyridin-4-yl). In some embodiments, R^{a3} is independently pyrimidinyl (e.g., pyrimidin-2-yl, pyrimidin-4-yl, pyrimidin-5-yl).

In some embodiments, R^{a3} is independently aryl. In some embodiments, R^{a3} is independently 6-10 member mono or bicyclic aryl. In some embodiments, R^{a3} is independently phenyl.

In some embodiments each R^{a3} is independently cycloalkylalkyl (e.g., cyclopropylmethyl, cyclobutylmethyl, cyclopentylmethyl, cyclohexylmethyl, cycloheptylmethyl). In some embodiments, each R^{a3} is independently heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, each R^{a3} is independently arylalkyl. In some embodiments, each R^{a3} is independently benzyl.

In some embodiments, each R^{a3} is independently heteroarylalkyl (e.g., pyridinylmethyl, thiazolylmethyl, triazolylmethyl, pyrazolylmethyl).

As generally defined herein, each R⁴ is selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂ wherein each alkyl, cycloalkyl, heterocyclyl, aryl, heteroaryl, arylalkyl and heteroarylalkyl is optionally substituted at any available position.

In some embodiments, each R⁴ is independently selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, —OR^{a4} and —N(R^{a4})₂, wherein each aryl, alkyl, cycloalkyl and heterocyclyl is substituted with 0, 1, 2 or 3 instances of halo (e.g., —F, —Cl), —OH, —CN, -Me, -Et, —NH₂ or oxo and wherein each R^{a4} is independently selected from H, —C₁-C₆ alkyl, —C₁-C₆ heteroalkyl, —C₁-C₆ haloalkyl and C₃-C₉ cycloalkyl.

In some embodiments, each R⁴ is independently selected from H, -D, halo (e.g., —F, —Cl), —CN, —C₁-C₆ alkyl (e.g., -Me, -Et, —Pr, -ⁱPr, -ⁿBu, -sec-Bu, -iso-Bu, -^{t}Bu), —C₁-C₆ alkenyl (e.g., vinyl, propenyl), —C₁-C₆ heteroalkyl (e.g., —CH₂NHCH2CH₃, —CH₂N(CH₃)CH₂CH₃, —CH₂N(CH₃)₂), —C₁-C₆ haloalkyl (e.g., —CF₃, —CHF2, —CH₂CF₃), —C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrrolidinyl, piperidinyl, piperazinyl, morpholinyl, 6-oxa-1-azaspiro[3.3]heptanyl, 6-oxa-1-azaspiro[3.4]octanyl), —C₆-C₁₀ aryl (e.g., phenyl), —OR^{a4} and —N(R^{a4})₂, wherein each aryl, alkyl, cycloalkyl and heterocyclyl is substituted with 0, 1, 2 or 3 instances of halo (e.g., —F, —Cl) or -Me, and wherein each R^{a4} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF2, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R⁴ is independently selected from H, -D, —CN, —C(═CH₂)CH₃, —C(CH₃)CH₂CH₃, —Cl, —F, -Me, -ⁱPr, —CH₂N(CH₃)CH₂CF₃, —CF₃, —CH₂CF₃, cyclopropyl (substituted with 0, 1 or 2 instances of —CN, —F, or -Me), azetidinyl (substituted with 0 or 1 instances of —F), phenyl (substituted with 0 or 1 instances of halo), —OCF₃, —OCH₂CF₃, —OCHF2, —OⁱPr, —OMe, —OCH₂CH(CH₃)₃, —N(Me)₂ and —NHMe and —NHⁱPr.

In some embodiments, each R⁴ is independently selected from H, -D, halo (e.g., —F, —Cl), —CN, —C₁-C₆ alkyl (e.g., -Me, -Et, —Pr, -ⁱPr, -ⁿBu, -sec-Bu, -iso-Bu, -^{t}Bu), —C₁-C₆ alkenyl (e.g., vinyl, propenyl), —C₁-C₆ heteroalkyl (e.g., —CH₂NHCH₂CH₃, —CH₂N(CH₃)CH₂CH₃, —CH₂N(CH₃)₂), —C₁-C₆ haloalkyl (e.g., —CF₃, —CHF₂, —CH₂CF₃), —C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrrolidinyl, piperidinyl, piperazinyl, morpholinyl, 6-oxa-1-azaspiro[3.3]heptanyl, 6-oxa-1-azaspiro[3.4]octanyl), —C₆-C₁₀ aryl (e.g., phenyl), —OR^{a4} and —N(R^{a4})₂, wherein each aryl, alkyl, cycloalkyl and heterocyclyl is substituted with 0, 1, 2 or 3 instances of halo (e.g., —F, —Cl), and wherein each R^{a4} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R⁴ is independently selected from H, -D, —CN, —C(═CH₂)CH₃, —C(CH₃)CH₂CH₃, —Cl, —F, -Me, -ⁱPr, —CH₂N(CH₃)CH₂CF₃, —CF₃, —CH₂CF₃, cyclopropyl (substituted with 0 or 1 instance of —CN), azetidinyl (substituted with 0 or 1 instances of —F), phenyl (substituted with 0 or 1 instances of halo), —OCF₃, —OCH₂CF₃, —OCHF₂, —OⁱPr, —OMe, —OCH₂CH(CH₃)₃, —N(Me)₂ and —NHMe and —NHⁱPr.

In certain embodiments, R⁴ is selected from H and —OMe.

In some embodiments, R⁴ is H. In some embodiments R⁴ is -D.

In certain embodiments, R⁴ is halo (e.g., fluoro, chloro, bromo, iodo). In some embodiments, R⁴ is —C₁. In some embodiments, R⁴ is —F. In some embodiments, R⁴ is —Br. In some embodiments, R⁴ is —I.

In some embodiments, R⁴ is —CN.

In certain embodiments, R⁴ is —C₁-C₆ alkyl (e.g., -Me, -Et, —Pr, -ⁱPr, -ⁿBu, -sec-Bu, -iso-Bu, -^{t}Bu, —C(CH₃)CH₂CH₃). In some embodiments, R⁴ is -Me. In some embodiments, R⁴ is -Et. In some embodiments R⁴ is —Pr. In some embodiments, R⁴ is -iPr. In some embodiments, R⁴ is —C(CH₃)CH₂CH₃.

In certain embodiments, R⁴ is —C₁-C₆ alkenyl (e.g., vinyl, propenyl). In some embodiments, R⁴ is vinyl. In some embodiments, R⁴ is propenyl (e.g., prop-1-en-1-yl, prop-1-en-2-yl). In some embodiments, R⁴ is prop-1-en-2-yl (—C(═CH₂)CH₃).

In some embodiments, R⁴ is —C₁-C₆ heteroalkyl. In some embodiments, R⁴ is methoxymethyl (—CH₂OCH₃). In some embodiments, R⁴ is aminomethyl (e.g., —CH₂NH₂, —CH₂NHCH₃, —CH₂NHCH₂CH₃, —CH₂N(CH₃)₂). In some embodiments, R⁴ is —CH₂N(CH₃)CH₂CH₃. In some embodiments, R⁴ is —CH₂N(CH₃)CH₂CF₃.

In some embodiments, R⁴ is —C₁-C₆ haloalkyl (e.g., —CF₃, —CHF₂, —CH₂CF₃). In some embodiments, R⁴ is trifluoromethyl (—CF₃). In other embodiments, R⁴ is difluoromethyl (—CHF₂).

In other embodiments, R⁴ is —CH₂CF₃.

In some embodiments, R⁴ is —C₁-C₆ hydroxyalkyl (e.g., —CH₂OH, —CH₂CH₂OH). In some embodiments, R⁴ is hydroxymethyl (—CH₂OH).

In some embodiments, R⁴ is optionally substituted —C₃-C₁₀ cycloalkyl (e.g., cyclopropyl, cyclobutyl, cyclopentyl, cyclohexyl). In some embodiments, R⁴ is optionally substituted cyclopropyl (e.g., substituted with 0, 1 or 2 instances of —CN, —F, or -Me). In some embodiments R⁴ is cyclobutyl. In some embodiments, R⁴ is cyclopentyl. In some embodiments, R⁴ is cyclohexyl.

In some embodiments, R⁴ is an optionally substituted 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl, 6-oxa-1-azaspiro[3.3]heptanyl, 6-oxa-1-azaspiro[3.4]octanyl). In some embodiments, R⁴ is oxetanyl. In some embodiments, R⁴ is tetrahydropyranyl. In some embodiments, R⁴ is tetrahydrofuranyl. In some embodiments, R⁴ is azetidinyl. In certain embodiments, the azetidinyl is optionally substituted (e.g., substituted with 0 or 1 instances of —F or -Me). In some embodiments, R⁴ is pyrrolidinyl. In some embodiments, R⁴ is piperidinyl. In some embodiments, R⁴ is piperazinyl. In some embodiments, R⁴ is morpholinyl. In some embodiments, R⁴ is azepanyl. In some embodiments, R⁴ is 6-oxa-1-azaspiro[3.3]heptanyl. In some embodiments, R⁴ is 6-oxa-1-azaspiro[3.4]octanyl.

In some embodiments R⁴ is cycloalkylalkyl (e.g., cyclopropylmethyl, cyclobutylmethyl, cyclopentylmethyl, cyclohexylmethyl, cycloheptylmethyl). In some embodiments, R⁴ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R⁴ is arylalkyl. In some embodiments, R⁴ is benzyl.

In some embodiments, R⁴ is heteroarylalkyl (e.g., pyridinylmethyl, thiazolylmethyl, triazolylmethyl, pyrazolylmethyl).

In some embodiments, R⁴ is optionally substituted —C₆-C₁₀ aryl (e.g., phenyl, naphthyl). In some embodiments, R⁴ is optionally substituted phenyl (e.g., phenyl substituted with 0 or 1 instances of halo (e.g., —Cl, —F)). In certain embodiments, R⁴ is -2-Cl-phenyl.

In some embodiments, R⁴ is —OR^{a4} (e.g., hydroxy (—OH), methoxy, difluoromethoxy (—OCHF₂), trifluoromethoxy (—OCF₃), —OCH(CH₃)CF₃, —OCH₂CF₃, ethoxy, propoxy, isopropoxy, cyclopropyloxy, cyclobutyloxy, —OCH₂CH(CH₃)₃). In some embodiments, R⁴ is hydroxy. In some embodiments, R⁴ is methoxy. In some embodiments, R⁴ is ethoxy. In some embodiments, R⁴ is propoxy. In some embodiments, R⁴ is isopropoxy. In some embodiments R⁴ is difluoromethoxy (—OCHF₂). In some embodiments, R⁴ is trifluoromethoxy (—OCF₃). In some embodiments, R⁴ is —OCH(CH₃)CF₃. In some embodiments, R⁴ is —OCH₂CF₃. In some embodiments, R⁴ is cyclopropyloxy. In some embodiments R⁴ is —OCH₂CH(CH₃)₃.

In some embodiments, R⁴ is —N(R^{a4})₂ (e.g., —NH₂, —NHR^{a4}, —N(CH₃)R^{a4}). In some embodiments, R⁴ is —NH₂. In some embodiments, R⁴ is —NHR^{a4} (e.g., —NHMe, -NHEt, —NHPr, —NHⁱPr, -NHcyclopropyl, -NHcyclobutyl). In some embodiments, R⁴ is —N(CH₃)R^{a4} (e.g., —NMe₂, —N(CH₃)Et, —N(CH₃)Pr, —N(CH₃)ⁱPr, —N(CH₃)cyclopropyl, —N(CH₃)cyclobutyl).

In some embodiments, R⁴ is —C(═O)R^{a4} or —C(═O)OR^{a4}. In some embodiments, R⁴ is —C(═O)R^{a4} wherein R^{a4} is as described herein. In some embodiments, R⁴ is —C(═O)alkyl. In some embodiments, R⁴ is —C(O)CH₃, —C(O)cyclopropyl, —C(O)cyclobutyl, —C(O)^{t}Bu, —C(O)ⁱPr, —C(O)Pr, —C(O)^{t}Bu, or —C(═O)OMe. In some embodiments, R⁴ is acetyl (—C(═O)Me). In some embodiments, R⁴ is —C(═O)OR^{a4}. In some embodiments, R⁴ is —COOH. In some embodiments, R⁴ is COOMe.

In some embodiments, R⁴ is —NR^{a4}C(═O)R^{a4}. In certain embodiments, R⁴ is —NHC(═O)R^{a4} (e.g., —NHC(═O)Me, —NHC(═O)Et, —NHC(═O)Pr, —NHC(═O)ⁱPr, —NHC(═O)Bu, —NHC(═O)^{t}Bu, —NHC(═O)Cyclopropyl, —NHC(═O)Cyclobutyl). In some embodiments, R⁴ is —N(CH₃)C(═O)R^{a4} (e.g., —N(CH₃)C(═O)Me, —N(CH₃)C(═O)Et, —N(CH₃)C(═O)Pr, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R⁴ is —NR^{a4}C(═O)OR^{a4}. In certain embodiments, R⁴ is —NHC(═O)OR^{a4} (e.g., —NHC(═O)OMe, —NHC(═O)OEt, —NHC(═O)OPr, —NHC(═O)OⁱPr, —NHC(═O)OBu, —NHC(═O)O^{t}Bu, —NHC(═O)OCyclopropyl, —NHC(═O)OCyclobutyl). In some embodiments, R⁴ is —N(CH₃)C(═O)OR^{a4} (e.g., —N(CH₃)C(═O)OMe, —N(CH₃)C(═O)OEt, —N(CH₃)C(═O)OPr, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R⁴ is —C(═O)N(R^{a4})₂ (e.g., —C(═O)NH₂, —C(═O)NHR^{a4}, C(═O)N(CH₃)R^{a4}). In some embodiments, R⁴ is —C(═O)NH₂. In certain embodiments, R⁴ is —C(═O)NHR^{a4} (e.g., —C(═O)NHMe, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R⁴ is —C(═O)N(CH₃)R^{a4} (e.g., —C(═O)NMe₂, —C(═O)N(CH₃)Et, —C(═O)N(CH₃)Pr, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R⁴ is —OC(═O)N(R^{a4})₂. In certain embodiments, R⁴ is —OC(═O)NHR^{a4} (e.g., —OC(═O)NHMe, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R⁴ is —OC(═O)N(CH₃)R^{a4} (e.g., —OC(═O)NMe2, —OC(═O)N(CH₃)Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R⁴ is —S(═O)R^{a4}. In certain embodiments, R⁴ is —S(═O)alkyl (e.g., —S(═O)Me, —S(═O)Et, —S(═O)Pr, —S(═O)ⁱPr). In certain embodiments, R⁴ is —S(═O)cycloalkyl (e.g., —S(═O)cyclopropyl, —S(═O)cyclobutyl, —S(═O)cyclopentyl, —S(═O)cyclohexyl).

In some embodiments, R⁴ is —S(═O)₂R^{a4}. In certain embodiments, R⁴ is —S(═O)₂alkyl (e.g., —S(═O)₂Me, —S(═O)₂Et, —S(═O)₂Pr, —S(═O)₂′Pr). In certain embodiments, R⁴ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁴ is S(═O)₂aryl (e.g., S(═O)₂phenyl).

In some embodiments, R⁴ is —SR^{a4}. In certain embodiments, R⁴ is -Salkyl (e.g., —SMe, -SEt, —SPr, —SⁱPr). In certain embodiments, R⁴ is -Scycloalkyl (e.g., -Scyclopropyl, -Scyclobutyl, -Scyclopentyl, -Scyclohexyl). In certain embodiments, R⁴ is -Saryl (e.g., Sphenyl).

In some embodiments, R⁴ is —S(═O)(═NR^{a4})R^{a4}. In certain embodiments, R⁴ is —S(═O)(═NH)R^{a4} (e.g., —S(═O)(═NH)Me, —S(═O)(═NH)Et, —S(═O)(═NH)Pr, —S(═O)(═NH) Pr, —S(═O)(═NH)Bu, —S(═O)(═NH)^{t}Bu, —S(═O)(═NH)Cyclopropyl, —S(═O)(═NH)Cyclobutyl). In some embodiments, R⁴ is —S(═O)(═NCH3)R^{a4} (e.g., —S(═O)(═NCH3)Me, —S(═O)(═NCH3)Et, —S(═O)(═NCH₃)Pr, —S(═O)(═NCH₃) Pr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁴ is —NR^{a4}S(═O)₂R^{a4}. In certain embodiments, R⁴ is —NHS(═O)₂alkyl (e.g., —NHS(═O)₂Me, —NHS(═O)₂Et, —NHS(═O)₂Pr, —NHS(═O)₂′Pr). In certain embodiments, R⁴ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R⁴ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂′Pr). In certain embodiments, R⁴ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R⁴ is —S(═O)₂N(R^{a4})₂. (e.g., —S(═O)₂NH₂, —S(═O)₂NHR^{a4}, S(═O)₂N(CH₃)R^{a4}). In some embodiments, R⁴ is —S(═O)₂NH₂. In some embodiments, R⁴ is —S(═O)₂NHR^{a4} (e.g., —S(═O)₂NHMe, —S(═O)₂NHEt, —S(═O)₂NHPr, —S(═O)₂NH′Pr, —S(═O)₂NHcyclopropyl, —S(═O)₂NHcyclobutyl). In some embodiments, R⁴ is —S(═O)₂N(CH₃)R^{a4} (e.g., —S(═O)₂NMe₂, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally defined herein, each R^{a4} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In some embodiments, each R^{a4} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, each R^{a4} is independently H.

In some embodiments, each R^{a4} is independently —C₁-C₆ alkyl (e.g., -Me, -Et, —Pr, -ⁱPr, -ⁿBu, -^{t}Bu, -sec-Bu, -iso-Bu). In some embodiments, each R^{a4} is independently -Me. In some embodiments, each R^{a4} is independently -Et. In some embodiments, each R^{a4} is independently —Pr. In some embodiments, each R^{a4} is independently -ⁱPr.

In some embodiments, each R^{a4} is independently —C₁-C₆ heteroalkyl. In some embodiments, each R^{a4} is independently methoxymethyl (—CH₂OCH₃). In some embodiments, each R^{a4} is independently aminomethyl (e.g., —CH₂NH₂, —CH₂NHCH3, —CH₂N(CH₃)₂.

In some embodiments, each R^{a4} is independently —C₁-C₆ haloalkyl. In some embodiments, each R^{a4} is independently trifluoromethyl (—CF₃). In other embodiments, each R^{a4} is independently difluoromethyl (—CHF₂). In some embodiments, each R^{a4} is —CH(CH₃)CF₃. In some embodiments, each R^{a4} is —CH₂CF₃.

In some embodiments, each R^{a4} is independently —C₃-C₉ cycloalkyl (e.g., cyclopropyl, cyclobutyl, cyclopentyl, cyclohexyl). In some embodiments, each R^{a4} is independently cyclopropyl. In some embodiments each R^{a4} is independently cyclobutyl. In some embodiments, each R^{a4} is independently cyclopentyl. In some embodiments, each R^{a4} is independently cyclohexyl.

In some embodiments, each R^{a4} is independently 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl).

In some embodiments, R^{a4} is independently heteroaryl. In some embodiments, R^{a4} is independently a 5-10 member heteroaryl (e.g., a 5-6 member monocyclic heteroaryl or an 8-10 member bicyclic heteroaryl containing 1-3 heteroatoms independently selected from N, O and S).

In some embodiments, R^{a4} is independently a 5-6 member monocyclic heteroaryl (e.g., a 5-member monocyclic heteroaryl containing 1-3 heteroatoms independently selected from O, N and S, a 6-member monocyclic heteroaryl containing 1-3 N heteroatoms). In some embodiments, R^{a4} is independently a 5-member monocyclic heteroaryl (e.g., pyrazolyl, pyrolyl, thiophenyl, furyl, thiazolyl, isothiazolyl, oxazolyl, isoxazolyl, imidazolyl, triazolyl, thiadiazolyl, oxadiazolyl). In some embodiments, R^{a4} is independently thiophenyl (e.g., thiophen-2-yl, thiophen-3-yl). In some embodiments, R^{a4} is independently pyrazolyl (e.g., pyrazol-1-yl, pyrazol-3-yl, pyrazol-5-yl). In some embodiments, R^{a4} is independently thiazolyl (e.g., thiazol-2-yl, thiazol-4-yl, thiazol-5-yl).

In some embodiments, R^{a4} is independently a 6-member monocyclic heteroaryl (e.g., pyridyl, pyrimidinyl, triazinyl, pyrazinyl, pyridazinyl). In some embodiments, R^{a4} is independently pyridinyl (e.g., pyridin-2-yl, pyridin-3-yl, pyridin-4-yl). In some embodiments, R^{a4} is independently pyrimidinyl (e.g., pyrimidin-2-yl, pyrimidin-4-yl, pyrimidin-5-yl).

In some embodiments, R^{a4} is independently aryl. In some embodiments, R^{a4} is independently 6-10 member mono or bicyclic aryl. In some embodiments, R^{a4} is independently phenyl.

In some embodiments each R^{a4} is independently cycloalkylalkyl (e.g., cyclopropylmethyl, cyclobutylmethyl, cyclopentylmethyl, cyclohexylmethyl, cycloheptylmethyl). In some embodiments, each R^{a4} is independently heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, each R^{a4} is independently arylalkyl. In some embodiments, each R^{a4} is independently benzyl.

In some embodiments, each R^{a4} is independently heteroarylalkyl (e.g., pyridinylmethyl, thiazolylmethyl, triazolylmethyl, pyrazolylmethyl).

In certain embodiments, the moiety represented by

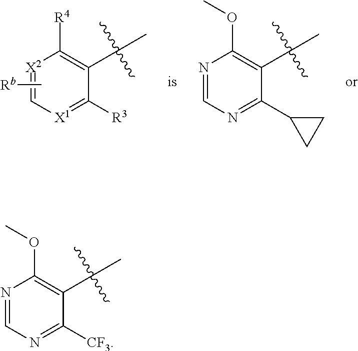

In some embodiments, the moiety represented by

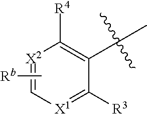

is selected from

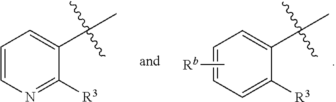

In some embodiments, the moiety represented by

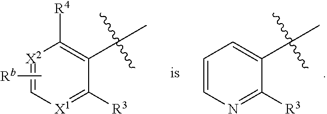

wherein R³ is as defined herein.

In some embodiments, R³ is selected from cyclopropyl, —OCH₂CF₃, —OCF₃, —OCHF₂, -ⁱPr and —OMe.

In some embodiments, the moiety represented by

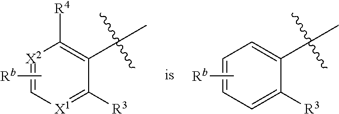

wherein R³ is as defined herein.

In some embodiments, R³ is selected from —Cl,-ⁱPr, —C(═CH₂)CH₃, —OCHF₂, —OCF₃, -2-Cl-phenyl, —CF₃ and cyclopropyl.

As generally defined herein, each R^{c} and R^{c′} is independently selected from H, —C₁-C₆ alkyl, —C₁-C₆ heteroalkyl, —C₁-C₆ haloalkyl, or R^{c} and R^{c′} can be taken together with the atom to which they are attached to form a —C₃-C₉ cycloalkyl or a carbonyl.

In some embodiments, R^{c} and R^{c′} are each independently selected from H and -Me, or are taken together to form a carbonyl group or a cyclopropyl group.

In some embodiments, R^{c} is H and R^{c′} is -Me.

In certain embodiments, R^{c} and R^{c′} are each independently H.

In certain embodiments, R^{c} and R^{c′} are each independently —C₁-C₆ alkyl (e.g., -Me, -Et, —Pr, -ⁱPr, -ⁿBu, -sec-Bu, -iso-Bu, -^{t}Bu). In some embodiments, R^{c} and R^{c′} are each independently -Me. In some embodiments, R^{c} and R^{c′} are each independently -Et. In some embodiments R^{c} and R^{c′} are each independently —Pr. In some embodiments, R^{c} and R^{c′} are each independently -iPr.

In some embodiments, R and R^{c′} are each independently —C₁-C₆ heteroalkyl. In some embodiments, R^{c} and R^{c′} are each independently methoxymethyl (—CH₂OCH₃). In some embodiments, R^{c} and R^{c′} are each independently hydroxymethyl (—CH₂OH). In some embodiments, R^{c} and R^{c′} are each independently aminomethyl (e.g., —CH₂NH₂, —CH₂NHCH₃, —CH₂N(CH₃)₂. In some embodiments, R^{c} and R^{c′} are each independently —CH₂N(CH₃)CH₂CH₃.

In some embodiments, R^{c} and R^{c′} are each independently —C₁-C₆ haloalkyl. In some embodiments, R^{c} and R^{c′} are each independently trifluoromethyl (—CF₃). In other embodiments, R^{c} and R^{c′} are each independently difluoromethyl (—CHF₂).

In some embodiments, R^{c} and R^{c′} are taken together with the carbon to which they are attached to form a carbonyl group (C(═O)).

In some embodiments, R^{c} and R^{c′} are taken together with the carbon to which they are attached to form a —C₃-C₉ cycloalkyl (e.g., cyclopropyl, cyclobutyl, cyclopentyl, cyclohexyl, cycloheptyl). In some embodiments, R^{c} and R^{c′} are taken together with the carbon to which they are attached to form a cyclopropyl. In some embodiments, R^{c} and R^{c′} are taken together with the carbon to which they are attached to form a cyclobutyl. In some embodiments, R^{c} and R^{c′} are taken together with the carbon to which they are attached to form a cyclopentyl. In some embodiments, R^{c} and R^{c′} are taken together with the carbon to which they are attached to form a cyclohexyl.

As generally defined herein, Ring A is selected from C₆-C₁₀ aryl, 5-10 membered heteroaryl, —C₃-C₁₀ cycloalkyl, and 3-10 membered heterocyclyl.

In certain embodiments, Ring A is selected from phenyl, pyridinyl (e.g., pyridin-2-yl, pyridin-3-yl), thiophenyl (e.g., thiophen-2-yl), cyclohexyl, piperidinyl (e.g., piperidin-4-yl, piperidin-2-yl) and piperazinyl.

In some embodiments, Ring A is a 6-membered heteroaryl containing 1-3 nitrogen atoms (e.g., pyridinyl, pyrimidinyl, pyrazinyl, triazinyl, pyridazinyl). In some embodiments, Ring A is pyridinyl (e.g., pyridin-2-yl, pyridin-3-yl, pyridin-4-yl). In some embodiments, Ring A is pyridin-2-yl.

In some embodiments, Ring A is a C₆-C₁₀ aryl (e.g., phenyl, naphthyl). In some embodiments, ring A is phenyl.

In some embodiments, Ring A is a 5-membered heteroaryl containing 1, 2 or 3 heteroatoms independently selected from N, O and S (e.g., furanyl, thiophenyl, pyrrolyl, pyrazolyl, thiazolyl, thiadiazolyl, oxazolyl, oxadiazolyl, triazolyl, thiadiazolyl, isothiazolyl, isoxazolyl, oxadiazolyl). In some embodiments, Ring A is thiophenyl (e.g., thiophen-2-yl, thiophen-3-yl). In some embodiments, Ring A is thiophen-2-yl.

In some embodiments, Ring A is a C₃-C₁₀ cycloalkyl (e.g., cyclopropyl, cyclobutyl, cyclopentyl, cyclohexyl, cycloheptyl). In some embodiments, ring A is cyclohexyl.

In other embodiments, Ring A a 3-10 membered heterocyclyl containing 1 or 2 heteroatoms selected from N, O and S (e.g., azetidinyl, oxetanyl, pyrrolidinyl, tetrahydrofuranyl, piperidinyl, piperazinyl, morpholinyl, thiomorpholinyl, azepanyl, diazepanyl). In some embodiments, Ring A is selected from piperidinyl and piperazinyl. In some embodiments, ring A is piperidinyl (e.g., piperidin-4-yl, piperidin-1-yl). In some embodiments, ring A is piperazinyl (e.g., piperazin-4-yl).

In some embodiments, the moiety represented by

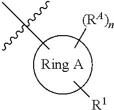

is selected from

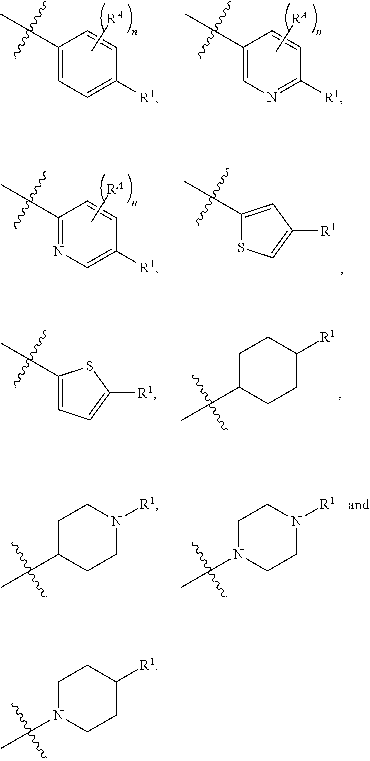

In certain embodiments, the moiety represented by

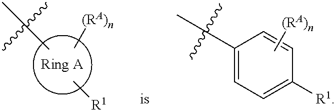

In certain embodiments, the moiety represented by

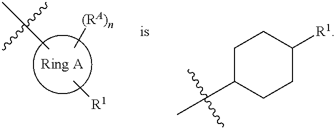

In some embodiments, the moiety represented by

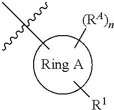

is selected from

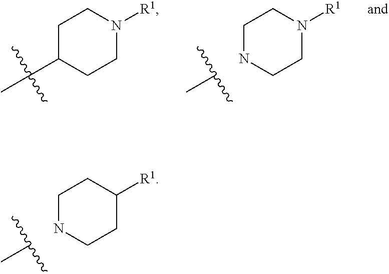

As generally defined herein, n is 0, 1, 2 or 3. In some embodiments, n is selected from 0, 1 or 2. In some embodiments n is 0. In some embodiments, n is 1 or 2. In some embodiments, n is 1. In some embodiments, n is 2.

As generally defined herein, each R^{A} is independently selected from -D, halo, —CN, —C₁-C₆ alkyl, —C₁-C₆ haloalkyl, —C₁-C₆ hydroxyalkyl, —C₃-C₁₀ cycloalkyl, —OR^{A1}, —N(R^{A1}), wherein each R^{A1} is independently selected from H, —C₁-C₆ alkyl, —C₁-C₆ haloalkyl and C₃-C₉ cycloalkyl. In certain embodiments, each R^{A} is independently selected from -D, halo (e.g., —F, —Cl), —C₁-C₆ alkyl (e.g., -Me, -Et, —Pr, -ⁱPr, -ⁿBu, -^{t}Bu), —OH and —O—C₁-C₆ alkyl (e.g., —OMe, -OEt, —OPr, —OⁱPr, -OⁿBu, -O^{t}Bu). In some embodiments, each R^{A} is independently selected from —F, —Cl, -Me, —OH and —OMe.

As generally defined herein, R¹ is an optionally substituted 5-10 membered heteroaryl or an optionally substituted 3-10 member heterocyclyl.

In certain embodiments, R¹ is a 5-10 membered heteroaryl or a 3-10 member heterocyclyl substituted with 0, 1, 2 or 3 instances of R⁵. In some embodiments, the heteroaryl or heterocyclyl is substituted with 0, 1 or 2 instances of R⁵. In some embodiments, the heteroaryl or heterocyclyl is substituted with 1 or 2 instances of R⁵. In some embodiments, the heteroaryl or heterocyclyl is substituted with 1 instance of R⁵. In some embodiments, the heteroaryl or heterocyclyl is substituted with 2 instances of R⁵.

In some embodiments, R¹ is a 3-7 member monocyclic heterocyclyl containing 1-3 heteroatoms selected from O, N and S (e.g., azetidinyl, oxetanyl, tetrahydrofuranyl, pyrrolidinyl, piperidinyl, piperazinyl, morpholinyl, thiomorpholinyl). In some embodiments, R¹ is 5-member monocyclic heterocyclyl (e.g., tetrahydrofuranyl, pyrrolidinyl). In some embodiments, R¹ is pyrrolidinyl (e.g., pyrrolidin-1-yl).

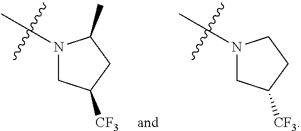 [SMILES: *N1C[C@H](C(F)(F)F)C[C@@H]1C ; "and" ; *N1CC[C@H](C(F)(F)F)C1]

In certain embodiments, R¹ is selected from CF₃ and CF₃.

In some embodiments, R¹ is an optionally substituted 5-6 member monocyclic heteroaryl containing 1-3 heteroatoms selected from O, N and S. In some embodiments, R¹ is substituted with 0, 1, 2 or 3 instances of R⁵. In some embodiments, R¹ is substituted with 0, 1 or 2 instances of R⁵. In some embodiments, R¹ is unsubstituted. In some embodiments, R¹ is substituted with 1 instance of R⁵. In some embodiments, R¹ is substituted with 2 instances of R⁵. In some embodiments, R¹ is substituted with 3 instances of R⁵.

In certain embodiments, R¹ is an optionally substituted 5 member monocyclic heteroaryl containing 1-3 heteroatoms selected from O, N and S. In some embodiments, R¹ is selected from pyrrolyl, pyrazolyl, imidazolyl, thiazolyl, furanyl, thiophenyl, oxazolyl, thiadiazolyl, oxadiazolyl, each substituted with 0, 1, 2 or 3 instances of R⁵. In some embodiments, R¹ is pyrrolyl (e.g., pyrrol-2-yl). In some embodiments, R¹ is pyrazolyl (e.g., pyrazol-1-yl, pyrazol-3-yl, pyrazol-4-yl, pyrazol-5-yl). In some embodiments, R¹ is pyrazol-1-yl. In some embodiments, R¹ is imidazolyl (e.g., imidazol-2-yl, imidazol-4-yl, imidazol-5-yl). In some embodiments, R¹ is imidazol-2-yl. In some embodiments, R¹ is thiazoly (e.g., thiazol-2-yl, thiazol-4-yl, thiazol-5-yl). In some embodiments, R¹ is furanyl (e.g., furan-2-yl, furan-3-yl). In some embodiments, R¹ is thiophenyl (e.g., thiophen-2-yl, thiophen-3-yl). In some embodiments, R¹ is oxazolyl (e.g., oxazol-2-yl, oxazol-4-yl, oxazol-5-yl). In some embodiments, R¹ is thiadiazolyl. In some embodiments, R¹ is oxadiazolyl. In some embodiments, R¹ is substituted with 0, 1 or 2 instances of R⁵. In some embodiments, R¹ is substituted with 1 or 2 instances of R⁵. In some embodiments, R¹ is unsubstituted. In some embodiments, R¹ is substituted with 1 instance of R⁵. In some embodiments, R¹ is substituted with 2 instances of R⁵. In some embodiments, R¹ is substituted with 3 instances of R⁵.

In certain embodiments, R¹ is selected from optionally substituted imidazolyl (e.g., imidazol-2-yl) and pyrazolyl (e.g., pyrazol-1-yl). In some embodiments, the imidazolyl and pyrazolyl are substituted with 1, 2 or 3 instances of R⁵. In some embodiments, the imidazolyl and pyrazolyl are substituted with 1 or 2 instances of R⁵.

In some embodiments, R¹ is imidazolyl (e.g., imidazol-2-yl) substituted with 0, 1, 2 or 3 instances of R⁵. In some embodiments, R¹ is imidazolyl (e.g., imidazol-2-yl) substituted with 0, 1 or 2 instances of R⁵. In some embodiments, R¹ is unsubstituted imidazolyl. In some embodiments, R¹ is imidazolyl substituted with one instance of R⁵. In some embodiments, R¹ is imidazolyl (e.g., imidazol-2-yl) substituted with 2 instances of R⁵. In some embodiments R¹ is imidazolyl (e.g., imidazol-2-yl) substituted with 3 instances of R⁵.

In some embodiments, R¹ is pyrazolyl (e.g., pyrazol-1-yl) substituted with 0, 1, 2 or 3 instances of R⁵. In some embodiments, R¹ is pyrazolyl (e.g., pyrazol-1-yl) substituted with 0, 1 or 2 instances of R⁵. In some embodiments, R¹ is unsubstituted pyrazolyl. In some embodiments, R¹ is pyrazolyl substituted with one instance of R⁵. In some embodiments, R¹ is pyrazolyl (e.g., pyrazol-1-yl) substituted with 2 instances of R⁵. In some embodiments, R¹ is pyrazolyl (e.g., pyrazol-1-yl) substituted with 3 instances of R⁵.

As generally defined herein, each R⁵ is independently selected from halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂ wherein each alkyl, cycloalkyl, heterocyclyl, aryl, heteroaryl, arylalkyl and heteroarylalkyl is optionally substituted at any available position.

In certain embodiments, R⁵ is selected from halo (e.g., —F, —Cl, —Br), —CN, —C₁-C₆ alkyl (e.g., -Me, -Et, —Pr, -ⁱPr, -ⁿBu, -^{t}Bu), —C₁-C₆ haloalkyl (e.g., —CF₃, —CHF₂, —CH₂CF₃, —CH₂CH₂F, —CH₂CHF₂), —OC₁-C₆ alkyl (e.g., —OMe, -OEt, —OPr, —OⁱPr, -O^{t}Bu, -O^{t}Bu), —C₃-C₁₀ cycloalkyl (e.g., cyclopropyl, cyclobutyl, cyclopentyl, cyclohexyl, cycloheptyl) and 3-10 membered heterocyclyl (e.g., azetidinyl, oxetanyl, tetrahydrofuranyl, tetrahydropyranyl, piperazinyl, piperidinyl, morpholinyl), wherein each alkyl, cycloalky and heterocyclyl is substituted with 0, 1 or 2 instances of -Me, —OMe, —OH, —CN, halo (e.g., —F, —Cl).

In certain embodiments, R⁵ is selected from —CN, —F, —Cl, —Br, -Me, -Et, -ⁱPr, —CF₃, —CH₂CH₂F, —CH₂CHF₂, —OMe, -OEt, —CH₂CH₂OMe, —CH₂CH₂OH, cyclopropyl, oxetanyl and azetidinyl (e.g., N-methyl-azetidin-3-yl).

In certain embodiments, R⁵ is halo (e.g., fluoro, chloro, bromo, iodo). In some embodiments, R⁵ is —Cl. In some embodiments, R⁵ is —F. In some embodiments, R⁵ is —Br. In some embodiments, R⁵ is —I.

In some embodiments, R⁵ is —CN.

In certain embodiments, R⁵ is —C₁-C₆ alkyl (e.g., -Me, -Et, —Pr, -ⁱPr, -ⁿBu, -sec-Bu, -iso-Bu, -^{t}Bu). In some embodiments, R⁵ is -Me. In some embodiments, R⁵ is -Et. In some embodiments R⁵ is —Pr. In some embodiments, R⁵ is -iPr.

In some embodiments, R⁵ is —C₁-C₆ heteroalkyl. In some embodiments, R⁵ is methoxymethyl (—CH₂OCH₃). In some embodiments R⁵ is —CH₂CH₂OMe. In some embodiments, R⁵ is aminomethyl (e.g., —CH₂NH₂, —CH₂NHCH₃, —CH₂N(CH₃)₂. In some embodiments, R⁵ is —CH₂N(CH₃)CH₂CH₃.

In some embodiments, R⁵ is —C₁-C₆ haloalkyl (e.g., —CF₃, —CHF₂, —CH₂CH₂F, —CH₂CHF₂). In some embodiments, R⁵ is trifluoromethyl (—CF₃). In other embodiments, R⁵ is difluoromethyl (—CHF₂). In some embodiments, R⁵ is —CH₂CH₂F. In other embodiments, R⁵ is —CH₂CHF₂.

In some embodiments, R⁵ is —C₁-C₆ hydroxyalkyl (e.g., —CH₂OH, —CH₂CH₂OH). In some embodiments, R⁵ is hydroxyethyl (—CH₂CH₂OH).

In some embodiments, R⁵ is —C₃-C₁₀ cycloalkyl (e.g., cyclopropyl, cyclobutyl, cyclopentyl, cyclohexyl). In some embodiments, R⁵ is cyclopropyl. In some embodiments R⁵ is cyclobutyl. In some embodiments, R⁵ is cyclopentyl. In some embodiments, R⁵ is cyclohexyl.

In some embodiments, R⁵ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl, 6-oxa-1-azaspiro[3.3]heptanyl, 6-oxa-1-azaspiro[3.4]octanyl). In some embodiments, R⁵ is oxetanyl. In some embodiments, R⁵ is tetrahydropyranyl. In some embodiments, R⁵ is tetrahydrofuranyl. In some embodiments, R⁵ is azetidinyl (e.g., N-methyl azetidin-3-yl). In some embodiments, R⁵ is pyrrolidinyl. In some embodiments, R⁵ is piperidinyl.

In some embodiments, R⁵ is piperazinyl. In some embodiments, R⁵ is morpholinyl. In some embodiments, R⁵ is azepanyl. In some embodiments, R⁵ is 6-oxa-1-azaspiro[3.3]heptanyl. In some embodiments, R⁵ is 6-oxa-1-azaspiro[3.4]octanyl.

In some embodiments R⁵ is cycloalkylalkyl (e.g., cyclopropylmethyl, cyclobutylmethyl, cyclopentylmethyl, cyclohexylmethyl, cycloheptylmethyl). In some embodiments, R⁵ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R⁵ is arylalkyl. In some embodiments, R⁵ is benzyl.

In some embodiments, R⁵ is heteroarylalkyl (e.g., pyridinylmethyl, thiazolylmethyl, triazolylmethyl, pyrazolylmethyl).

In some embodiments, R⁵ is —OR^{a5} (e.g., hydroxy (—OH), methoxy, difluoromethoxy (—OCHF2), trifluoromethoxy (—OCF₃), —OCH(CH₃)CF₃, —OCH₂CF₃, ethoxy, propoxy, isopropoxy, cyclopropyloxy, cyclobutyloxy,). In some embodiments, R⁵ is hydroxy. In some embodiments, R⁵ is methoxy. In some embodiments, R⁵ is ethoxy. In some embodiments, R⁵ is propoxy. In some embodiments, R⁵ is isopropoxy. In some embodiments R⁵ is difluoromethoxy (—OCHF₂). In some embodiments, R⁵ is trifluoromethoxy (—OCF₃). In some embodiments, R⁵ is —OCH(CH₃)CF₃. In some embodiments, R⁵ is —OCH₂CF₃. In some embodiments, R⁵ is cyclopropyloxy.

In some embodiments, R⁵ is —N(R^{a5})₂ (e.g., —NH₂, —NHR^{a5}, —N(CH₃)R^{a5}). In some embodiments, R⁵ is —NH₂. In some embodiments, R⁵ is —NHR^{a5} (e.g., —NHMe, -NHEt, —NHPr, -NHⁱPr, -NHcyclopropyl, -NHcyclobutyl). In some embodiments, R⁵ is —N(CH₃)R^{a5} (e.g., —NMe₂, —N(CH₃)Et, —N(CH₃)Pr, —N(CH₃)ⁱPr, —N(CH₃)cyclopropyl, —N(CH₃)cyclobutyl).

In some embodiments, R⁵ is —C(═O)R^{a}s or —C(═O)OR^{a}s. In some embodiments, R⁵ is —C(═O)R^{a}s wherein R^{a5} is as described herein. In some embodiments, R⁵ is —C(═O)alkyl. In some embodiments, R⁵ is —C(O)CH₃, —C(O)cyclopropyl, —C(O)cyclobutyl, —C(O)^{t}Bu, —C(O)ⁱPr, —C(O)Pr, —C(O)^{t}Bu, or —C(═O)OMe. In some embodiments, R⁵ is acetyl (—C(═O)Me). In some embodiments, R⁵ is —C(═O)OR^{a5}. In some embodiments, R⁵ is —COOH. In some embodiments, R⁵ is COOMe.

In some embodiments, R⁵ is —NR^{a5}C(═O)R^{a5}. In certain embodiments, R⁵ is —NHC(═O)R^{a5} (e.g., —NHC(═O)Me, —NHC(═O)Et, —NHC(═O)Pr, —NHC(═O)ⁱPr, —NHC(═O)Bu, —NHC(═O)^{t}Bu, —NHC(═O)Cyclopropyl, —NHC(═O)Cyclobutyl). In some embodiments, R⁵ is —N(CH₃)C(═O)R^{a5} (e.g., —N(CH₃)C(═O)Me, —N(CH₃)C(═O)Et, —N(CH₃)C(═O)Pr, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R⁵ is —NR^{a5}C(═O)OR^{a5}. In certain embodiments, R⁵ is —NHC(═O)OR^{a5} (e.g., —NHC(═O)OMe, —NHC(═O)OEt, —NHC(═O)OPr, —NHC(═O)OⁱPr, —NHC(═O)OBu, —NHC(═O)O^{t}Bu, —NHC(═O)OCyclopropyl, —NHC(═O)OCyclobutyl). In some embodiments, R⁵ is —N(CH₃)C(═O)OR^{a}s (e.g., —N(CH₃)C(═O)OMe, —N(CH₃)C(═O)OEt, —N(CH₃)C(═O)OPr, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R⁵ is —C(═O)N(R^{a5})₂ (e.g., —C(═O)NH₂, —C(═O)NHR^{a5}, C(═O)N(CH₃)R^{a5}). In some embodiments, R⁵ is —C(═O)NH₂. In certain embodiments, R⁵ is —C(═O)NHR^{a5} (e.g., —C(═O)NHMe, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R⁵ is —C(═O)N(CH₃)R^{a5} (e.g., —C(═O)NMe₂, —C(═O)N(CH₃)Et, —C(═O)N(CH₃)Pr, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R⁵ is —OC(═O)N(R^{a5})₂. In certain embodiments, R⁵ is —OC(═O)NHR^{a5} (e.g., —OC(═O)NHMe, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R⁵ is —OC(═O)N(CH₃)R^{a}s (e.g., —OC(═O)NMe₂, —OC(═O)N(CH₃)Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R⁵ is —S(═O)R^{a5}. In certain embodiments, R⁵ is —S(═O)alkyl (e.g., —S(═O)Me, —S(═O)Et, —S(═O)Pr, —S(═O)ⁱPr). In certain embodiments, R⁵ is —S(═O)cycloalkyl (e.g., —S(═O)cyclopropyl, —S(═O)cyclobutyl, —S(═O)cyclopentyl, —S(═O)cyclohexyl).

In some embodiments, R⁵ is —S(═O)₂R^{a5}. In certain embodiments, R⁵ is —S(═O)₂alkyl (e.g., —S(═O)₂Me, —S(═O)₂Et, —S(═O)₂Pr, —S(═O)₂′Pr). In certain embodiments, R⁵ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁵ is S(═O)₂aryl (e.g., S(═O)₂phenyl).

In some embodiments, R⁵ is —SR^{a5}. In certain embodiments, R⁵ is -Salkyl (e.g., —SMe, -SEt, —SPr, —SⁱPr). In certain embodiments, R⁵ is -Scycloalkyl (e.g., -Scyclopropyl, -Scyclobutyl, -Scyclopentyl, -Scyclohexyl). In certain embodiments, R⁵ is -Saryl (e.g., Sphenyl).

In some embodiments, R⁵ is —S(═O)(═NR^{a5})R^{a}s. In certain embodiments, R⁵ is —S(═O)(═NH)R^{a5} (e.g., —S(═O)(═NH)Me, —S(═O)(═NH)Et, —S(═O)(═NH)Pr, —S(═O)(═NH) Pr, —S(═O)(═NH)Bu, —S(═O)(═NH)^{t}Bu, —S(═O)(═NH)Cyclopropyl, —S(═O)(═NH)Cyclobutyl). In some embodiments, R⁵ is —S(═O)(═NCH₃)R^{a5} (e.g., —S(═O)(═NCH₃)Me, —S(═O)(═NCH₃)Et, —S(═O)(═NCH₃)Pr, —S(═O)(═NCH₃) Pr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁵ is —NR^{a5}S(═O)₂R^{a5}. In certain embodiments, R⁵ is —NHS(═O)₂alkyl (e.g., —NHS(═O)₂Me, —NHS(═O)₂Et, —NHS(═O)₂Pr, —NHS(═O)₂′Pr). In certain embodiments, R⁵ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R⁵ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂′Pr). In certain embodiments, R⁵ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R⁵ is —S(═O)₂N(R^{a5})₂. (e.g., —S(═O)₂NH₂, —S(═O)₂NHR^{a5}, S(═O)₂N(CH₃)R^{a5}). In some embodiments, R⁵ is —S(═O)₂NH₂. In some embodiments, R⁵ is —S(═O)₂NHR^{a5} (e.g., —S(═O)₂NHMe, —S(═O)₂NHEt, —S(═O)₂NHPr, —S(═O)₂NH′Pr, —S(═O)₂NHcyclopropyl, —S(═O)₂NHcyclobutyl). In some embodiments, R⁵ is —S(═O)₂N(CH₃)R^{a}s (e.g., —S(═O)₂NMe₂, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally defined herein, each R^{a}s is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In some embodiments, each R^{a5} is independently selected from H, —C₁-C₆ alkyl (e.g., -Me, -Et, —Pr, -ⁱPr, -ⁿBu, -^{t}Bu, -sec-Bu, -iso-Bu) and —C₁-C₆ haloalkyl (e.g., —CHF₂, —CF₃, —CH(CH₃)CF₃, —CH₂CF₃).

In some embodiments, each R^{a5} is independently H.

In some embodiments, each R^{a5} is independently —C₁-C₆ alkyl (e.g., -Me, -Et, —Pr, -ⁱPr, -ⁿBu, -^{t}Bu, -sec-Bu, -iso-Bu). In some embodiments, each R^{a5} is independently -Me. In some embodiments, each R^{a5} is independently -Et. In some embodiments, each R^{a5} is independently —Pr. In some embodiments, each R^{a5} is independently -ⁱPr.

In some embodiments, each R^{a5} is independently —C₁-C₆ heteroalkyl. In some embodiments, each R^{a5} is independently methoxymethyl (—CH₂OCH₃). In some embodiments, each R^{a5} is independently hydroxymethyl (—CH₂OH). In some embodiments, each R^{a}s is independently aminomethyl (e.g., —CH₂NH₂, —CH₂NHCH₃, —CH₂N(CH₃)₂.

In some embodiments, each R^{a5} is independently —C₁-C₆ haloalkyl. In some embodiments, each R^{a5} is independently trifluoromethyl (—CF₃). In other embodiments, each R^{a5} is independently difluoromethyl (—CHF₂). In some embodiments, each R^{a5} is —CH(CH₃)CF₃. In some embodiments, each R^{a5} is —CH₂CF₃.

In some embodiments, each R^{a5} is independently —C₃-C₉ cycloalkyl (e.g., cyclopropyl, cyclobutyl, cyclopentyl, cyclohexyl). In some embodiments, each R^{a5} is independently cyclopropyl. In some embodiments each R^{a5} is independently cyclobutyl. In some embodiments, each R^{a5} is independently cyclopentyl. In some embodiments, each R^{a5} is independently cyclohexyl.

In some embodiments, each R^{a5} is independently 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl).

In some embodiments, R^{a5} is independently heteroaryl. In some embodiments, R^{a5} is independently a 5-10 member heteroaryl (e.g., a 5-6 member monocyclic heteroaryl or an 8-10 member bicyclic heteroaryl containing 1-3 heteroatoms independently selected from N, O and S).

In some embodiments, R^{a}s is independently a 5-6 member monocyclic heteroaryl (e.g., a 5-member monocyclic heteroaryl containing 1-3 heteroatoms independently selected from O, N and S, a 6-member monocyclic heteroaryl containing 1-3 N heteroatoms). In some embodiments, R^{a5} is independently a 5-member monocyclic heteroaryl (e.g., pyrazolyl, pyrolyl, thiophenyl, furyl, thiazolyl, isothiazolyl, oxazolyl, isoxazolyl, imidazolyl, triazolyl, thiadiazolyl, oxadiazolyl). In some embodiments, R^{a5} is independently thiophenyl (e.g., thiophen-2-yl, thiophen-3-yl). In some embodiments, R^{a5} is independently pyrazolyl (e.g., pyrazol-1-yl, pyrazol-3-yl, pyrazol-5-yl). In some embodiments, R^{a5} is independently thiazolyl (e.g., thiazol-2-yl, thiazol-4-yl, thiazol-5-yl).

In some embodiments, R^{a5} is independently a 6-member monocyclic heteroaryl (e.g., pyridyl, pyrimidinyl, triazinyl, pyrazinyl, pyridazinyl). In some embodiments, R^{a5} is independently pyridinyl (e.g., pyridin-2-yl, pyridin-3-yl, pyridin-4-yl). In some embodiments, R^{a5} is independently pyrimidinyl (e.g., pyrimidin-2-yl, pyrimidin-4-yl, pyrimidin-5-yl).

In some embodiments, R^{a5} is independently aryl. In some embodiments, R^{a5} is independently 6-10 member mono or bicyclic aryl. In some embodiments, R^{a5} is independently phenyl.

In some embodiments each R^{a5} is independently cycloalkylalkyl (e.g., cyclopropylmethyl, cyclobutylmethyl, cyclopentylmethyl, cyclohexylmethyl, cycloheptylmethyl). In some embodiments, each R^{a5} is independently heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, each R^{a5} is independently arylalkyl. In some embodiments, each R^{a5} is independently benzyl.

In some embodiments, each R^{a5} is independently heteroarylalkyl (e.g., pyridinylmethyl, thiazolylmethyl, triazolylmethyl, pyrazolylmethyl).

In some embodiments, R¹ is selected from:

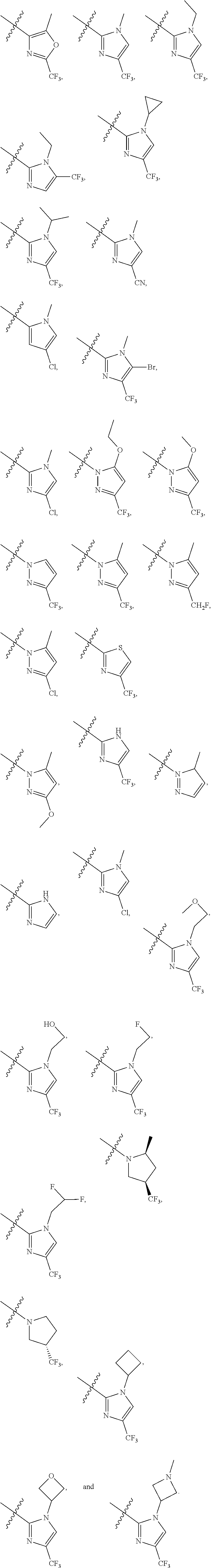

In some embodiments, R¹ is selected from:

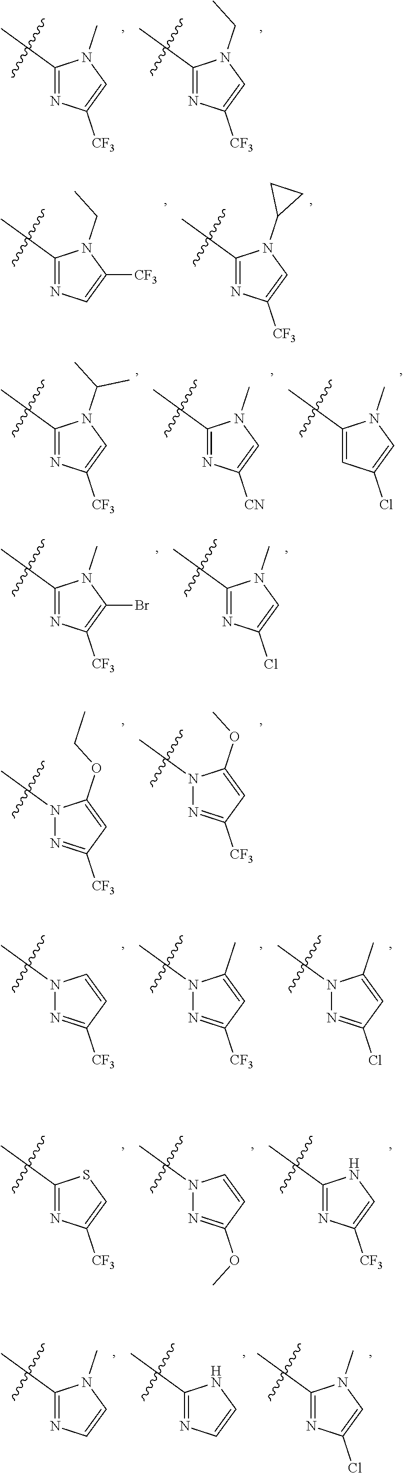

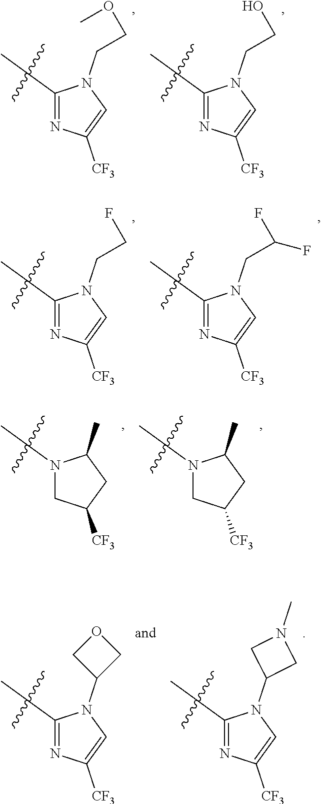

In some embodiments, R¹ is selected from

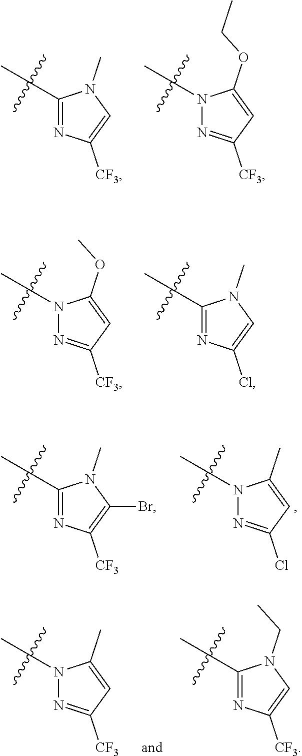

As generally defined herein, R² is selected from H, —C₁-C₆ alkyl, —C₁-C₆ haloalkyl, —C₁-C₆ heteroalkyl, —C₁-C₆ hydroxyalkyl, —C₃-C₁₀ cycloalkyl and arylalkyl, wherein each hydrogen of the alkyl, haloalkyl, heteroalkyl, hydroxylalkyl and arylalkyl can be independently replaced with a deuterium atom.

In some embodiments, R² is selected from —C₁-C₆ alkyl (e.g., -Me, -Et, —Pr, -ⁱPr, -ⁿBu, -^{t}Bu), —C₁-C₆ haloalkyl (e.g., —CF₃, —CHF₂, —CH₂CHF₂, —CH₂CF₃), —C₁-C₆ heteroalkyl (e.g., —CH₂CH₂OMe), —C₃-C₁₀ cycloalkyl (e.g., cyclopropyl, cyclobutyl, cyclopentyl, cyclohexyl) wherein each hydrogen of the alkyl, haloalkyl and heteroalkyl can be independently replaced with a deuterium atom.

In certain embodiments, R² is selected from -Me, -Et, —CH₂CHF₂, —CH₂CF₃, cyclobutyl and —CH₂CH₂OMe.

—C₁-C₆ alkyl wherein one or more of the hydrogen atoms of the alkyl are replaced with a deuterium atom. (e.g., -CD₃, -CD₂CD₃). In some embodiments, R² is -CD₃.

In some embodiments, R² is H or -Me.

In certain embodiments, R² is —C₁-C₆ alkyl (e.g., -Me, -Et, —Pr, -ⁱPr, -ⁿBu, -sec-Bu, -iso-Bu, -^{t}Bu). In some embodiments, R² is -Me. In some embodiments, R² is -Et. In some embodiments R² is —Pr. In some embodiments, R² is -iPr.

In certain embodiments, R² is —C₁-C₆ alkyl (e.g., -Me, -Et, —Pr, -ⁱPr, -ⁿBu, -sec-Bu, -iso-Bu, -^{t}Bu) wherein one or more of the hydrogen atoms of the alkyl are replaced with a deuterium atom. (e.g., -CD₃, -CD₂CD₃). In some embodiments, R² is -CD₃.

In some embodiments, R² is —C₁-C₆ heteroalkyl. In some embodiments, R² is methoxymethyl (—CH₂OCH₃). In some embodiments, R² is aminomethyl (e.g., —CH₂NH₂, —CH₂NHCH₃, —CH₂N(CH₃)₂. In some embodiments, R² is —CH₂N(CH₃)CH₂CH₃.

In some embodiments, R² is —C₁-C₆ haloalkyl. In some embodiments, R² is trifluoromethyl (—CF₃). In other embodiments, R² is difluoromethyl (—CHF₂).

In some embodiments, R² is —C₁-C₆ hydroxyalkyl (e.g., —CH₂OH, —CH₂CH₂OH). In some embodiments, R² is hydroxymethyl (—CH₂OH).

In some embodiments, R² is arylalkyl. In some embodiments, R² is benzyl.

As generally defined herein, R⁶ is H, -D, halo, —CN, —C₁-C₆ alkyl, —C₁-C₆ alkynyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, 6-10 member heteroaryl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, and —OC(═O)N(R^{a6})₂, wherein each alkyl, alkynyl, cycloalkyl, heterocyclyl, aryl, heteroaryl, arylalkyl and heteroarylalkyl is optionally substituted at any available position.

In certain embodiments, R⁶ is selected from H, -D, —CN, halo (e.g., —F, —Cl), —C₁-C₆ alkyl (e.g., -Me, -Et, —Pr, -ⁱPr, -ⁿBu, -^{t}Bu), —C₁-C₆ haloalkyl (e.g., —CF₃, —CHF₂, —CH₂CF₃), —C₁-C₆ alkynyl (e.g., —CCH, —CC—CH₃, —CC-cyclopropyl), —C₆-C₁₀ aryl (e.g., phenyl substituted with 0-1 instances of C₁-C₆ alkyl), —C(═O)N(R^{a6})₂ (e.g., —C(═O)NMe₂, —C(═O)NHMe, —C(═O)NH₂), —C₃-C₁₀ cycloalkyl (e.g., cyclopropyl, cyclobutyl, cyclopentyl, cyclohexyl), 6-10 member heteroaryl (e.g., pyridinyl), —N(R^{a6})₂, (e.g., —NH₂, —NMe₂, —NHMe), —OH, and —O(C₁-C₆ alkyl) (e.g., —OMe).

In some embodiments, R⁶ is selected from H, -D, —CN, —F, —Cl, -Me, -Et, —Pr, -ⁱPr, -ⁿBu, -^{t}Bu, —CF₃, —CHF₂, phenyl (e.g., 2-ⁱPr-phenyl), -pyridinyl (e.g., 2-pyridinyl), —CC—CH₃, —CC-cyclopropyl, —C(═O)NMe₂, —C(═O)NHMe, —C(═O)NH₂, —NH₂, —NMe₂, —NHMe, —OH and —OMe. In some embodiments, R⁶ is selected from H, —Cl, -Me and —CF₃. In some embodiments, R⁶ is H.

In some embodiments, R⁶ is D.

In certain embodiments, R⁶ is halo (e.g., fluoro, chloro, bromo, iodo). In some embodiments, R⁶ is —C₁. In some embodiments, R⁶ is —F. In some embodiments, R⁶ is —Br. In some embodiments, R⁶ is —I.

In some embodiments, R⁶ is —CN.

In certain embodiments, R⁶ is —C₁-C₆ alkyl (e.g., -Me, -Et, —Pr, -ⁱPr, -ⁿBu, -sec-Bu, -iso-Bu, -^{t}Bu). In some embodiments, R⁶ is -Me. In some embodiments, R⁶ is -Et. In some embodiments R⁶ is —Pr. In some embodiments, R⁶ is -iPr.

In some embodiments, R⁶ is —C₁-C₆ heteroalkyl. In some embodiments, R⁶ is methoxymethyl (—CH₂OCH₃). In some embodiments, R⁶ is hydroxymethyl (—CH₂OH). In some embodiments, R⁶ is aminomethyl (e.g., —CH₂NH₂, —CH₂NHCH3, —CH₂N(CH₃)₂. In some embodiments, R⁶ is —CH₂N(CH₃)CH₂CH₃.

In some embodiments, R⁶ is —C₁-C₆ haloalkyl. In some embodiments, R⁶ is trifluoromethyl (—CF₃). In other embodiments, R⁶ is difluoromethyl (—CHF2).

In some embodiments, R⁶ is —C₃-C₁₀ cycloalkyl (e.g., cyclopropyl, cyclobutyl, cyclopentyl, cyclohexyl). In some embodiments, R⁶ is cyclopropyl. In some embodiments R⁶ is cyclobutyl. In some embodiments, R⁶ is cyclopentyl. In some embodiments, R⁶ is cyclohexyl.

In some embodiments, R⁶ is hydroxy (—OH). In certain embodiments, R⁶ is —O(C₁-C₆ alkyl) (e.g., methoxy, ethoxy, propoxy, isopropoxy). In some embodiments, R⁶ is methoxy. In some embodiments, R⁶ is ethoxy. In some embodiments, R⁶ is propoxy. In some embodiments, R⁶ is isopropoxy.

As generally defined herein, each R^{a6} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

In some embodiments, R^{a6} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF2, —CH₂CF₃, —CH(CH₃)CF₃) and C₃-C₉ cycloalkyl (e.g., cyclopropyl, cyclobutyl, cyclopentyl, cyclohexyl, cycloheptyl).

In some embodiments, the compound is selected from the compounds of Table 1.

In some embodiments, provided is a pharmaceutical composition comprising a compound of Formula (I), (II) or a compound of Table 1 or a pharmaceutically acceptable salt, hydrate, solvate, prodrug, tautomer or stereoisomer thereof as defined herein and a pharmaceutically acceptable carrier. In some embodiments, the pharmaceutical composition further comprises a second therapeutic agent.

In various embodiments, the Compounds of the Disclosure are USP1 inhibitors that reduce the level of USP1 protein and/or inhibit or reduce at least one biological activity of USP 1 protein.

In some embodiments, the Compounds of the Disclosure specifically bind to USP1 protein. In some embodiments, the Compounds of the Disclosure specifically bind to USP1 protein in a USP1-UAF1 complex. In some embodiments, the Compounds of the Disclosure specifically bind to USP1 mRNA. In some embodiments, the Compounds of the Disclosure specifically bind to USP1 protein (alone or in a USP1-UAF1 complex) or USP1 mRNA. In some embodiments, the Compounds of the Disclosure specifically bind to UAF1 (alone or in a USP1-UAF1 complex) and inhibit or reduces formation or activity of the USP1-UAF1 complex.

In some embodiments, the Compounds of the Disclosure decrease the formation of the USP1-UAF1 complex. In some embodiments, the Compounds of the Disclosure decrease the activity of the USP1-UAF1 complex. In some embodiments, the Compounds of the Disclosure decrease the deubiquitinase activity of USP1. In some embodiments, the Compounds of the Disclosure increase mono-ubiquitinated PCNA. In some embodiments, the Compounds of the Disclosure increase mono-ubiquitinated FANCD2. In some embodiments, the Compounds of the Disclosure increase mono-ubiquitinated FANCI.

In some embodiments, the Compounds of the Disclosure do not bind to other deubiquitinases, other USP proteins, or other UAF1 complexes (e.g., USP46-UAF1) or bind deubiquitinases, other USP proteins, or other UAF1 complexes (e.g., USP46-UAF1) with at least 5-fold, at least 10-fold, at least 20-fold, or at least 100-fold reduced affinity compared to the affinity for USP1 (i.e., the KD of the USP1 inhibitor for other deubiquitinases, other USP proteins, or other UAF1 complexes (e.g., USP46-UAF1) is at least 5-fold, at least 10-fold, at at least 20 fold, least 50 fold, at least 100 fold.

Certain compounds of the disclosure were assessed for USP1-UAF1 activity in a Ubiquitin Rhodamine assay as described in Biology Example 1.

Table 1 indicates IC₅₀ values (μM) against USP1-UAF1 for exemplary compounds (column 4). For column 4, “a” indicates an IC₅₀ value lower than 30 nM, “b” indicates an IC₅₀ value equal to or greater than 30 nM and lower than 100 nM, “c” indicates an IC₅₀ value equal to or greater than 100 nM but lower than 10 μM, and “d” indicates an IC₅₀ value equal to or greater than 10 μM.

Table 1 also indicates IC₅₀ values in a viability assay for a non-isogenic pair of BRCA1 mutant (column 5-MDA-MB-436) and BRCA1 WT (column 6—HCC1954) cell lines. These values indicate the effect of treatment with compound on cell survival. In columns 5 and 6, a value of “aa” and “aaa” indicates an IC₅₀ of less than 100 nM in the mutant and wild-type cell lines, respectively; a value of “bb” and “bbb” indicates an IC₅₀ equal to or greater than 100 nM but less than 250 nM in the mutant and wild-type cell lines, respectively; a value of “cc” and “ccc” indicates an IC₅₀ equal to or greater than 250 nM but less than 10 μM in the mutant and wild-type cell lines, respectively; a value of “dd” and “ddd” indicates an IC₅₀ greater than or equal to 10 μM in the mutant and wild-type cell lines, respectively.

Table 1 also indicates IC₅₀ values for exemplary compounds in an AlphaLISA assay measuring monoubiquitinated PCNA in a BRCA1 mutant cell line (MDA-MB-436; column 7). In column 7, a value of “A” indicates an IC₅₀ of less than 100 nM, a value of “B” indicates an IC₅₀ equal to or greater than 100 nM but less than 250 nM, a value of “C” indicates an IC₅₀ equal to or greater than 250 nM but less than 10 μM, a value of “D” indicates an IC₅₀ greater than or equal to 10 μM.

Unless otherwise indicated, the absolute stereochemistry of all chiral atoms is as depicted. Compounds marked with (or) or (rel) are single enantiomers wherein the absolute stereochemistry was arbitrarily assigned (e.g., based on chiral SFC elution as described in the Examples section). Compounds marked with (and) or (rac) are mixtures of enantiomers wherein the relative stereochemistry is as shown. Compounds marked with (abs) are single enantiomers wherein the absolute sterochemistry is as indicated.

TABLE 1
Exemplary compounds and biological data

| | | | USP1- | MDA-MB- | | Alpha |
|---|---|---|---|---|---|---|
| | | | UAF1 | 436 rel | HCC1954 | Lisa |
| Structure | Nr | Stereo | IC50 | IC50 | rel IC50 | IC50 |
| 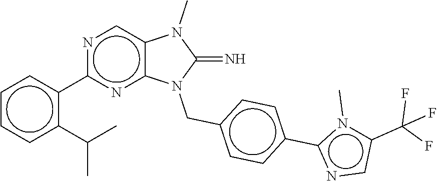 | A-1 | | c | cc | ccc | C |
| 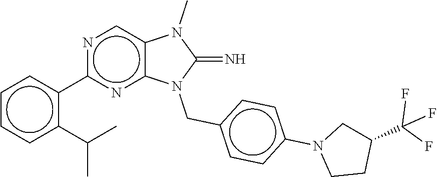 | A-10d | or | c | | | |
| 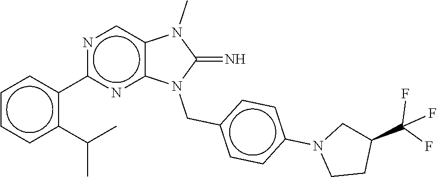 | A-10e | or | c | | | |
| 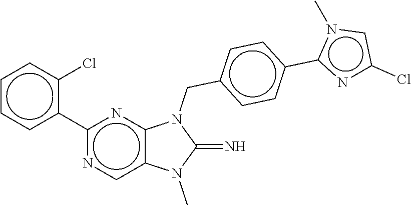 | A-11 | | c | cc | ddd | C |
| 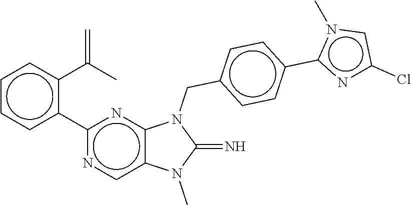 | A-12 | | a | aa | ddd | A |
| 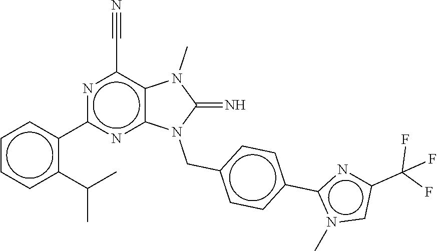 | A-13 | | c | cc | ccc | C |
| 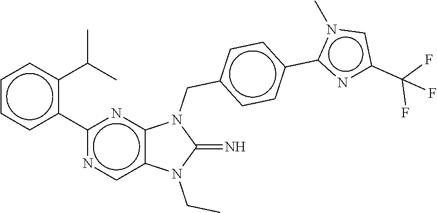 | A-14 | | a | aa | ccc | A |
| 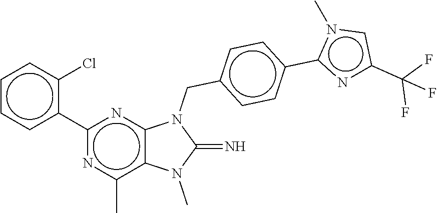 | A-15a | | b | cc | ddd | C |
| 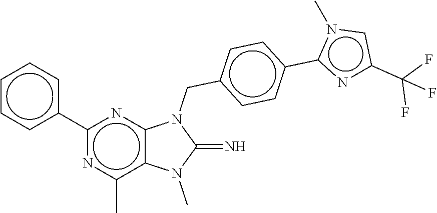 | A-15b | | c | cc | ccc | |
| 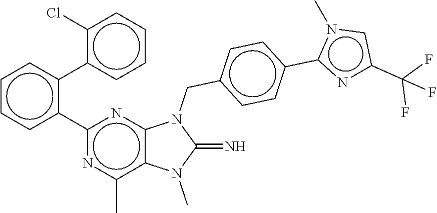 | A-15c | | a | bb | ccc | A |
| 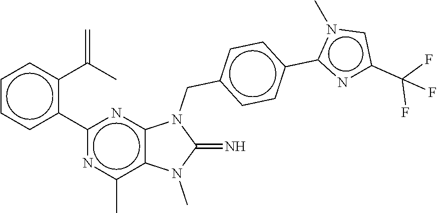 | A-16 | | a | aa | ccc | A |
| 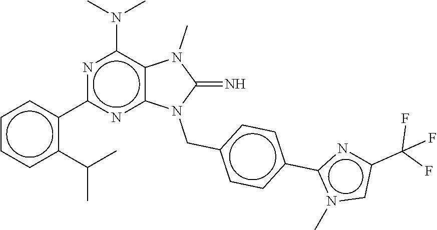 | A-17 | | c | cc | ccc | C |
| 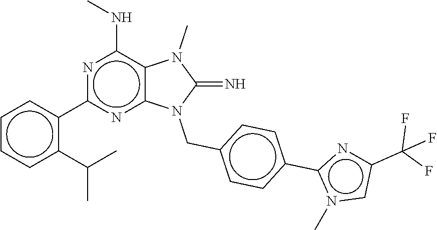 | A-18 | | c | cc | ccc | D |
| 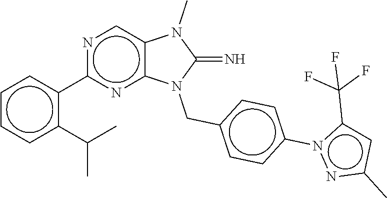 | A-19a | | | | | |
| 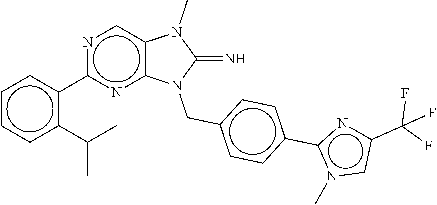 | A-19b | | a | aa | ccc | A |
| 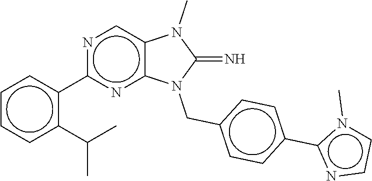 | A-2 | | c | cc | ddd | |
| 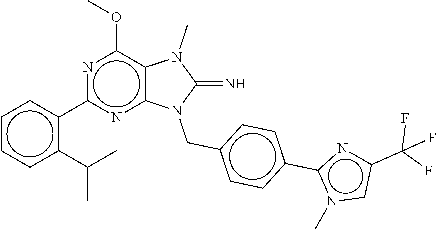 | A-20 | | c | cc | ccc | |
| 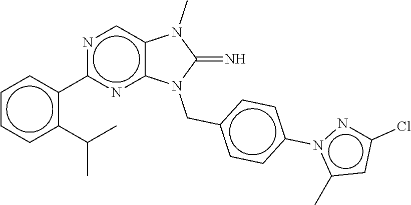 | A-21 | | a | aa | ccc | A |
| 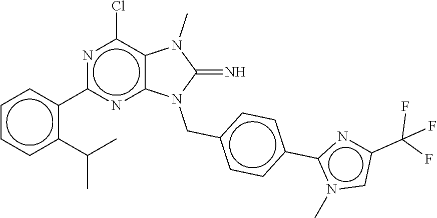 | A-22a | | a | cc | ccc | C |
| 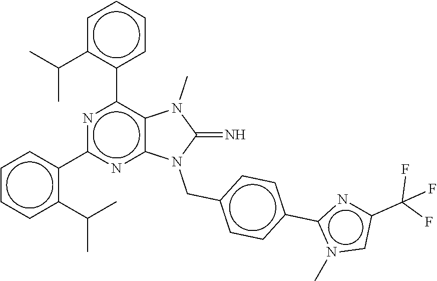 | A-22b | | b | cc | ccc | C |
| 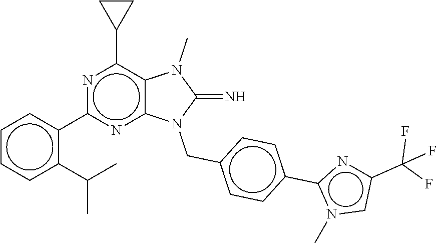 | A-23 | | a | cc | ccc | C |
| 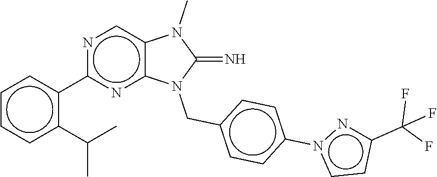 | A-24 | | b | cc | ccc | C |
| 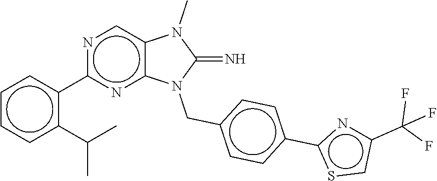 | A-25 | | b | cc | ccc | C |
| 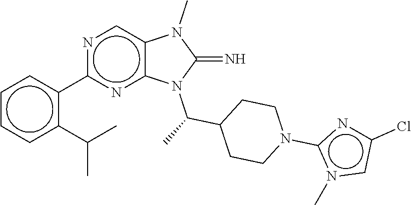 | A-26c | or | c | cc | ccc | C |
| 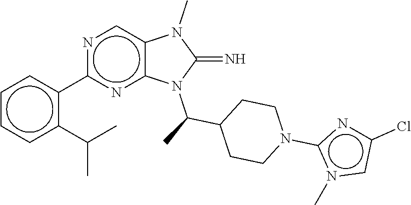 | A-26d | or | c | cc | ddd | |
| 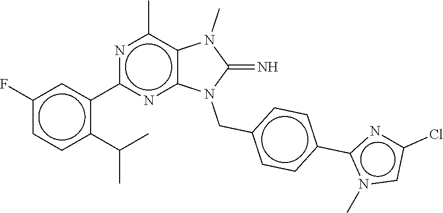 | A-27 | | a | cc | ccc | B |
| 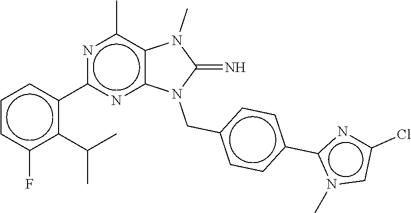 | A-28 | | a | aa | ddd | A |
| 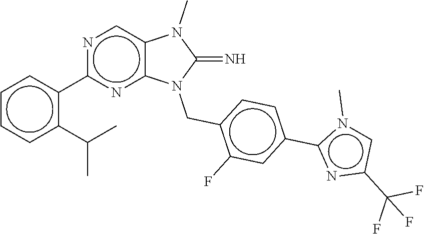 | A-29 | | a | aa | ddd | A |
| 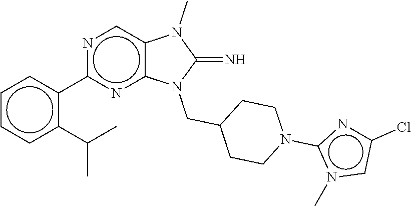 | A-3 | | c | cc | ddd | C |
| 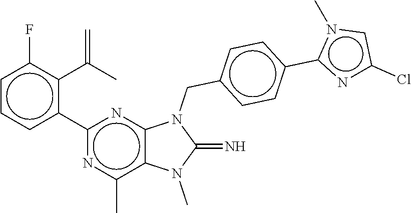 | A-30 | | a | aa | ccc | A |
| 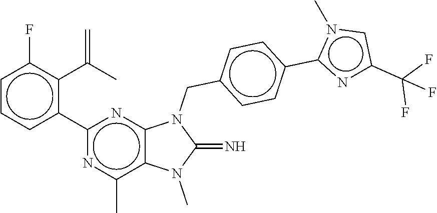 | A-31 | | a | aa | ccc | A |
| 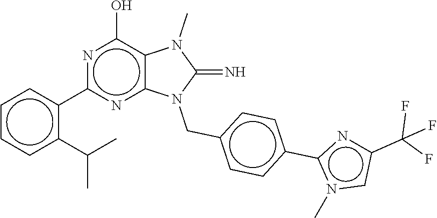 | A-32 | | c | cc | ddd | |
| 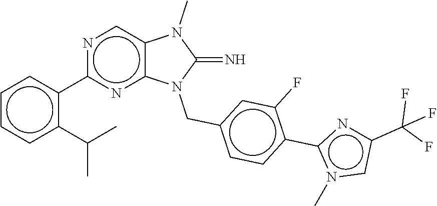 | A-33 | | a | | | A |
| 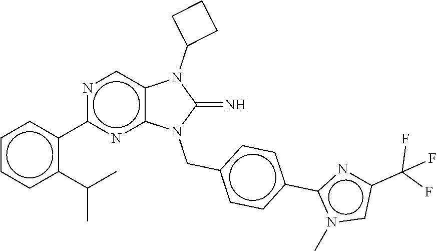 | A-34 | | a | bb | ccc | A |
| 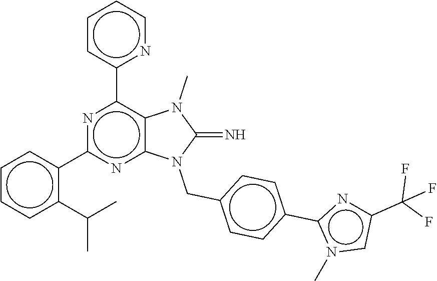 | A-35 | | b | cc | ccc | B |
| 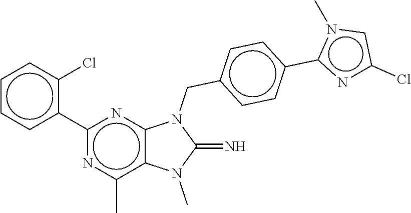 | A-36 | | c | cc | ddd | C |
| 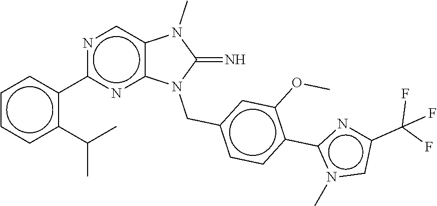 | A-37 | | a | cc | ddd | B |
| 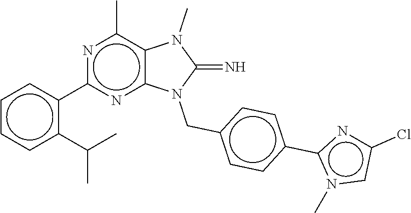 | A-38 | | a | aa | ccc | A |
| 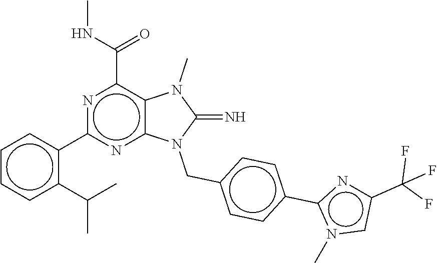 | A-39 | | c | cc | ddd | |
| 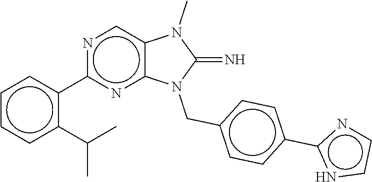 | A-4 | | c | | | |
| 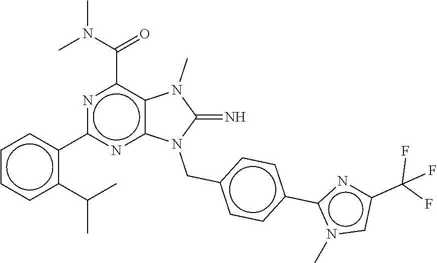 | A-40 | | c | cc | ddd | |
| 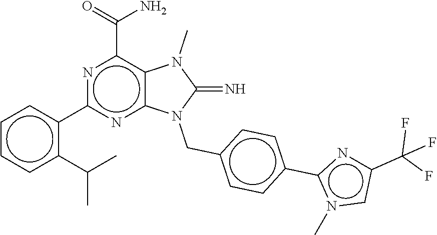 | A-41 | | b | cc | ccc | C |
| 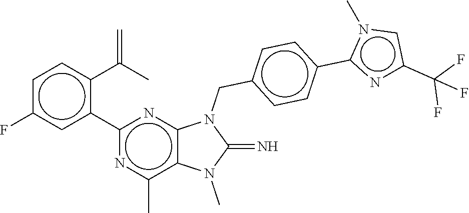 | A-42 | | b | cc | ccc | B |
| 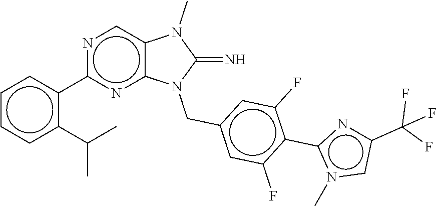 | A-43 | | b | | | C |
| 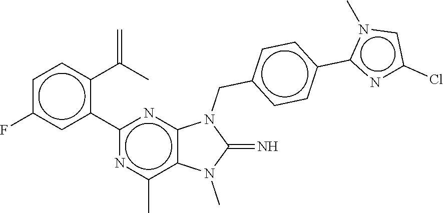 | A-44 | | b | cc | ccc | C |
| 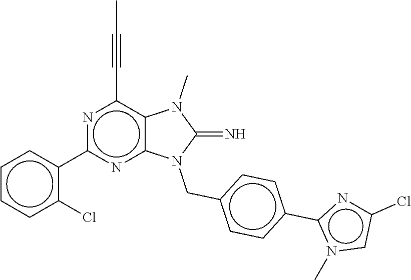 | A-45 | | c | cc | ccc | |
| 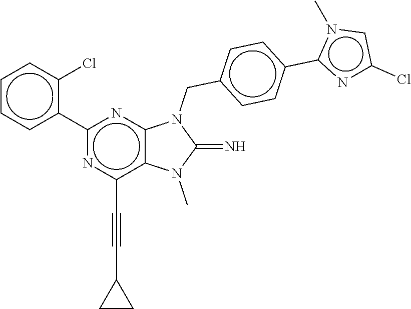 | A-46 | | c | cc | ccc | |
| 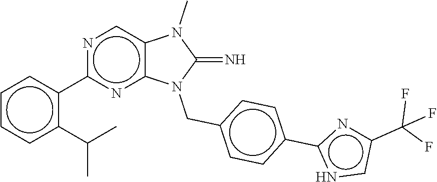 | A-5 | | b | cc | ccc | C |
| 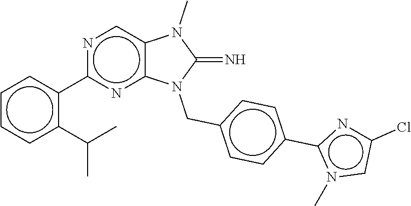 | A-6 | | a | aa | ddd | A |
| 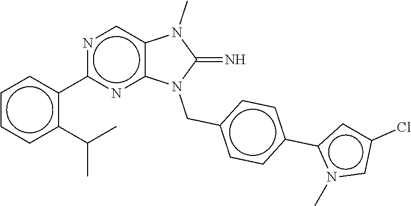 | A-7 | | c | cc | ccc | |
| 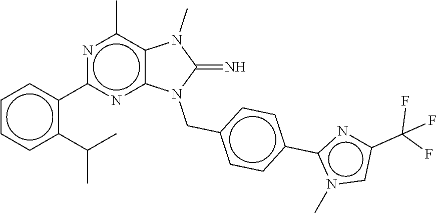 | A-8 | | a | aa | ccc | A |
| 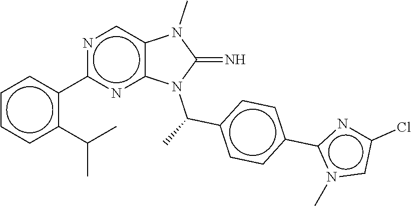 | A-9c | or | b | cc | ddd | B |
| 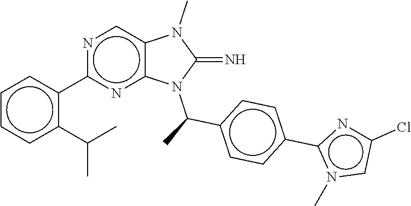 | A-9d | or | a | aa | ccc | A |
| 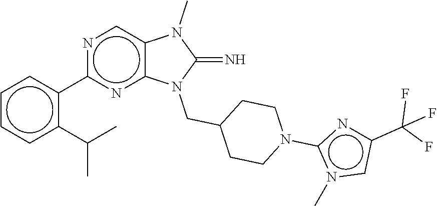 | B-1 | | c | bb | ccc | B |
| 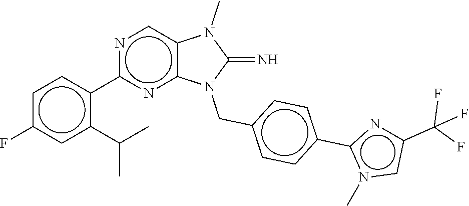 | B-10 | | a | bb | ccc | B |
| 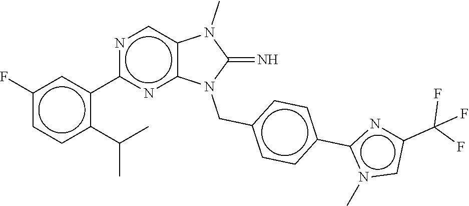 | B-11 | | a | bb | ccc | A |
| 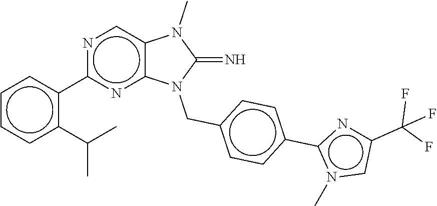 | B-2 | | a | aa | ddd | A |
| 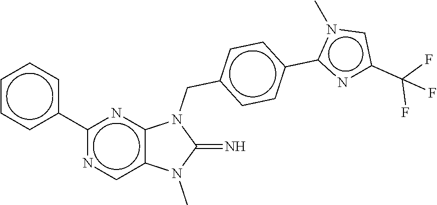 | B-3 | | c | cc | ccc | C |
| 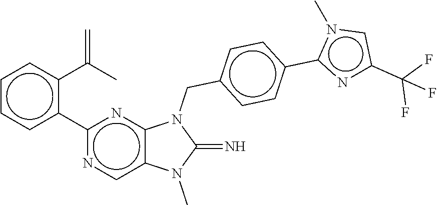 | B-4 | | a | aa | ddd | A |
| 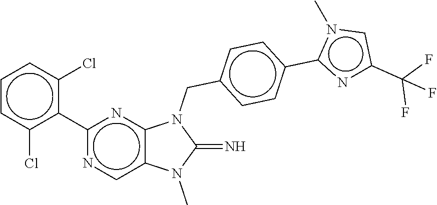 | B-5 | | a | bb | ccc | B |
| 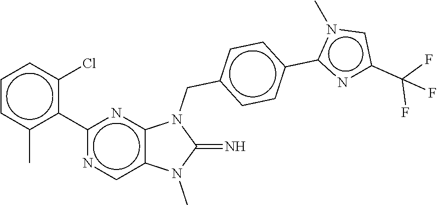 | B-7 | | b | | | |
| 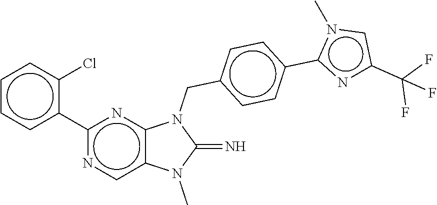 | B-8 | | b | bb | ddd | B |
| 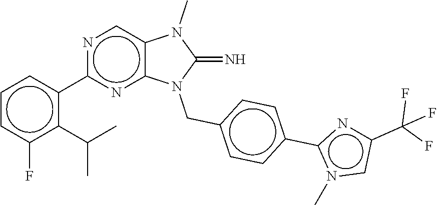 | B-9 | | a | aa | ccc | A |
| 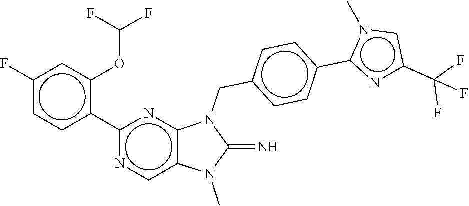 | T-001 | | b | cc | ccc | C |
| 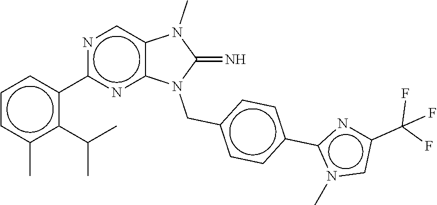 | T-002 | | b | bb | ccc | C |
| 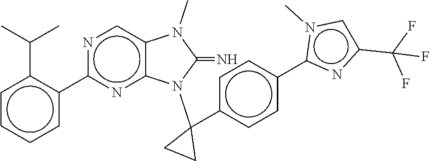 | T-003 | | a | aa | ccc | A |
| 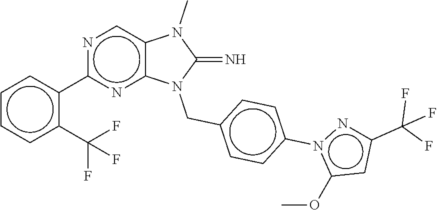 | T-004 | | b | cc | ccc | B |
| 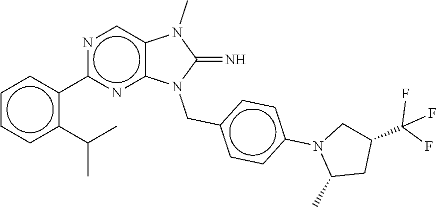 | T-005 | or | | | | |
| 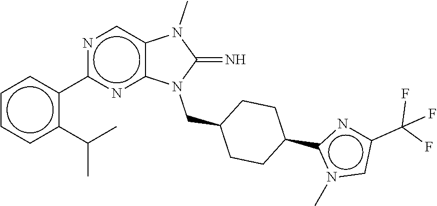 | T-006 | rel | c | cc | ccc | |
| 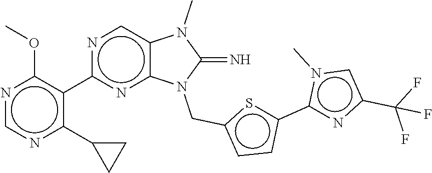 | T-007 | | c | cc | ddd | |
| 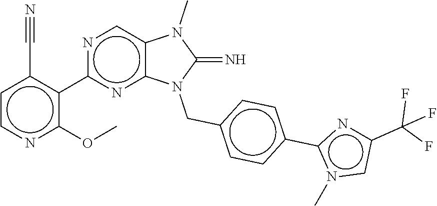 | T-008 | | b | bb | ddd | B |
| 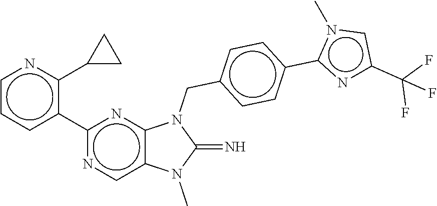 | T-009 | | b | cc | ddd | B |
| 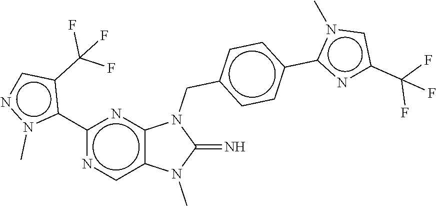 | T-010 | | b | cc | | B |
| 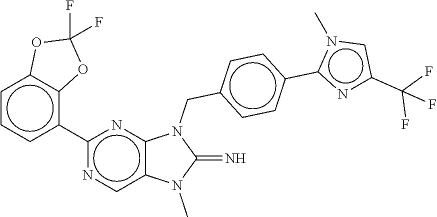 | T-011 | | b | cc | ccc | C |
| 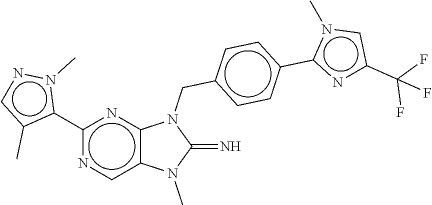 | T-012 | | c | cc | ddd | |
| 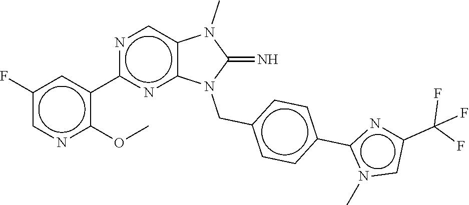 | T-013 | | c | cc | ddd | C |
| 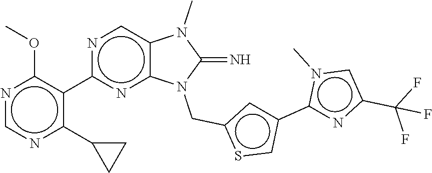 | T-014 | | c | cc | ddd | |
| 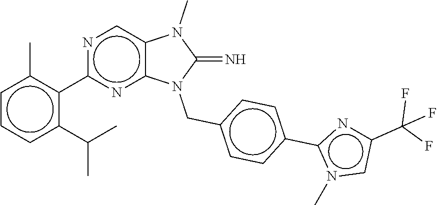 | T-015 | | a | bb | ddd | B |
| 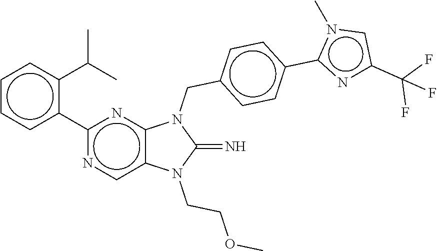 | T-016 | | a | aa | ddd | A |
| 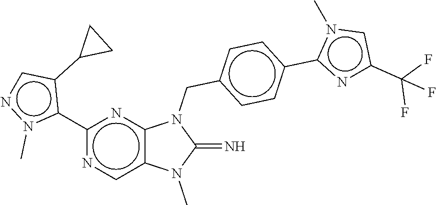 | T-017 | | c | cc | ddd | |
| 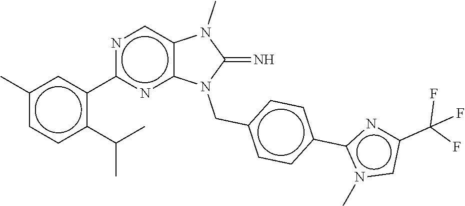 | T-018 | | b | cc | ccc | C |
| 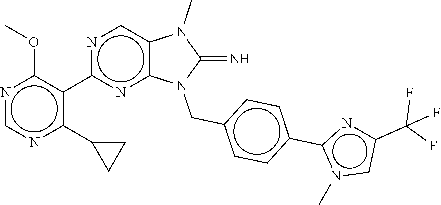 | T-019 | | c | bb | ddd | A |
| 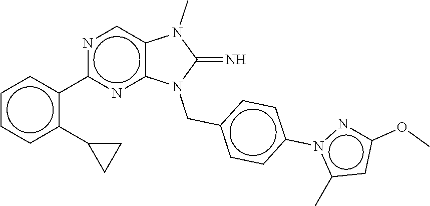 | T-020 | | c | cc | ccc | C |
| 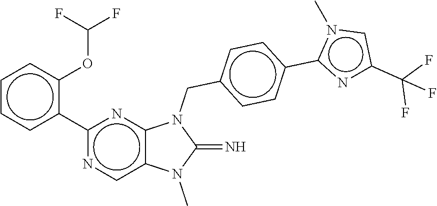 | T-021 | | b | bb | ddd | A |
| 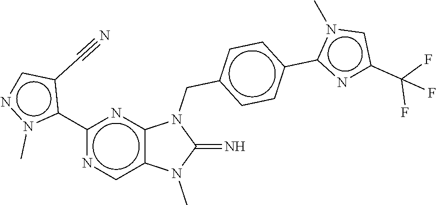 | T-022 | | c | cc | ddd | |
| 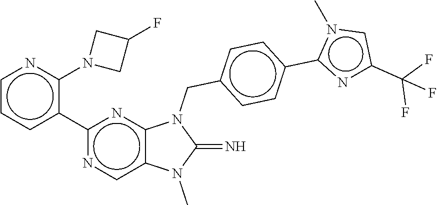 | T-023 | | c | cc | ddd | C |
| 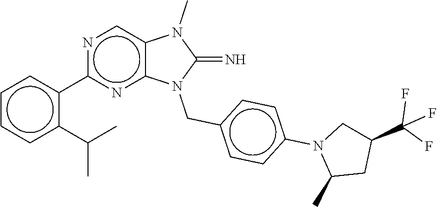 | T-024 | or | c | | | |
| 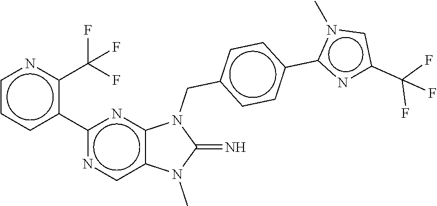 | T-025 | | c | cc | ddd | |
| 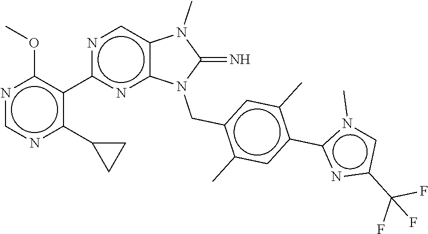 | T-026 | | c | cc | ddd | |
| 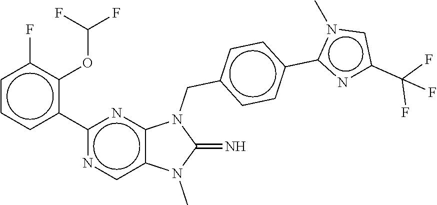 | T-027 | | b | cc | ccc | C |
| 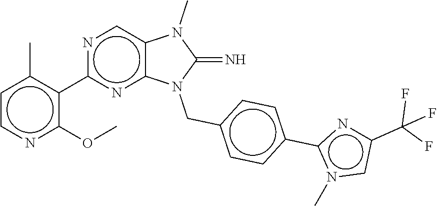 | T-028 | | c | cc | ddd | |
| 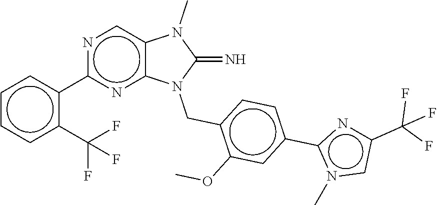 | T-029 | | c | bb | ccc | B |
| 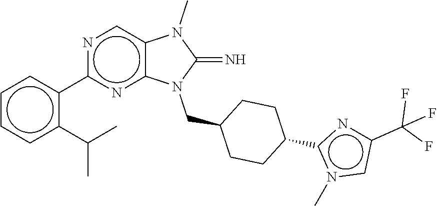 | T-030 | rel | c | aa | ccc | A |
| 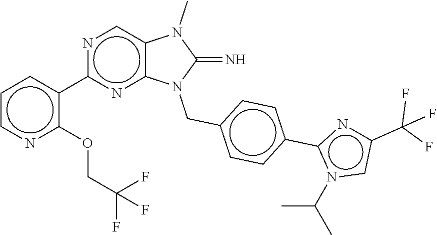 | T-031 | | c | cc | ddd | C |
| 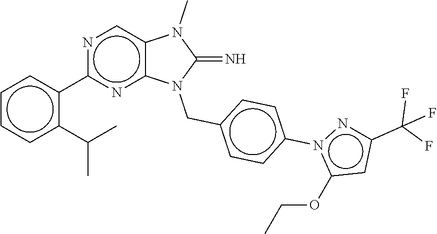 | T-032 | | a | aa | ccc | B |
| 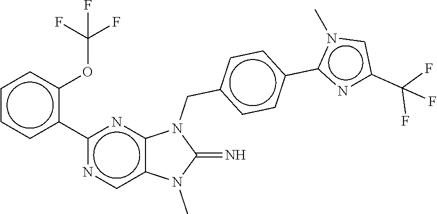 | T-033 | | a | aa | ccc | A |
| 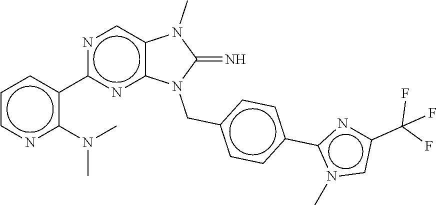 | T-034 | | c | cc | ddd | C |
| 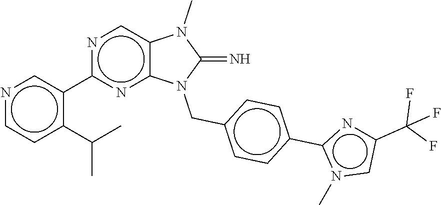 | T-035 | | c | cc | ddd | C |
| 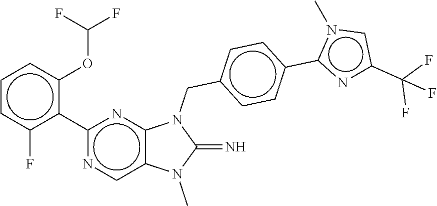 | T-036 | | a | aa | ddd | A |
| 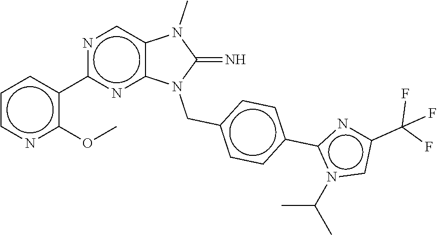 | T-037 | | c | cc | ddd | C |
| 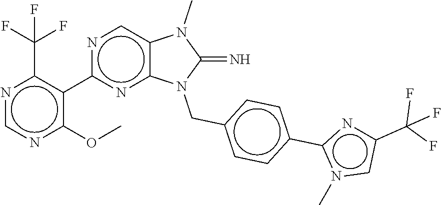 | T-038 | | b | bb | ddd | A |
| 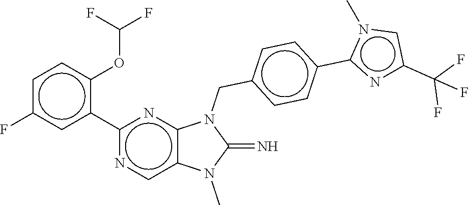 | T-039 | | b | cc | ddd | C |
| 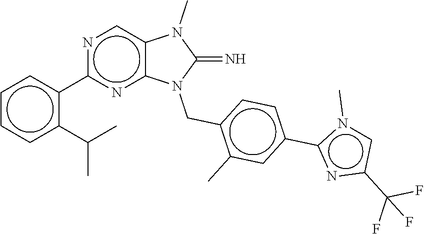 | T-040 | | b | aa | ddd | A |
| 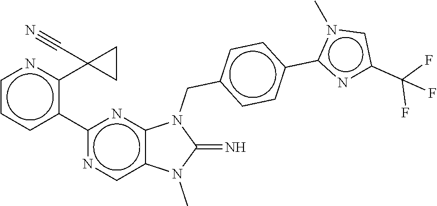 | T-041 | | c | cc | ddd | |
| 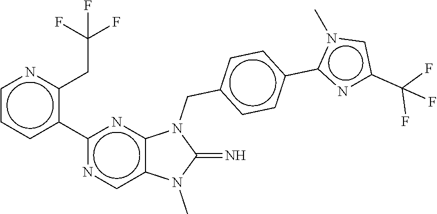 | T-042 | | c | cc | ddd | |
| 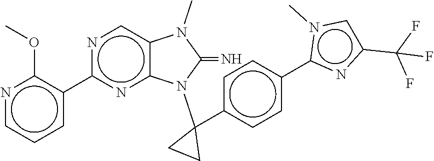 | T-043 | | c | cc | ddd | |
| 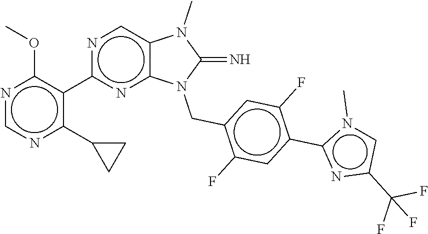 | T-044 | | b | cc | ddd | B |
| 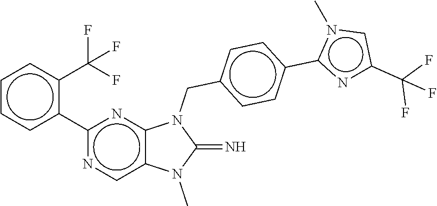 | T-045 | | a | aa | | B |
| 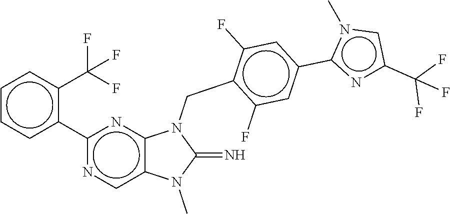 | T-046 | | c | cc | ddd | |
| 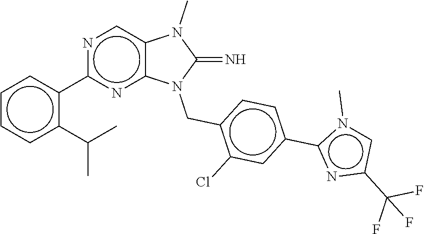 | T-047 | | b | cc | ddd | B |
| 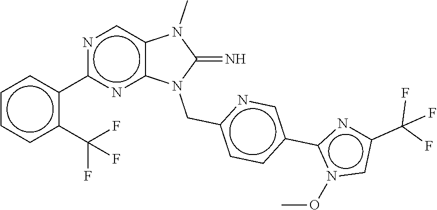 | T-048 | | c | cc | ddd | |
| 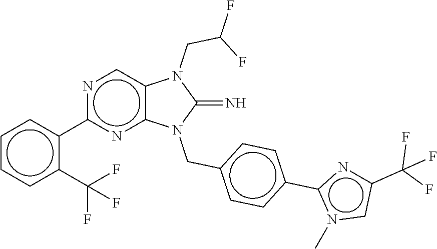 | T-049 | | b | bb | ddd | C |
| 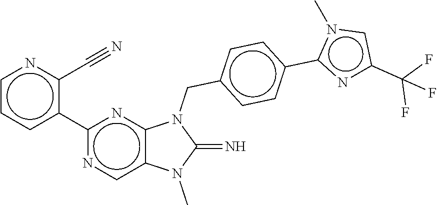 | T-050 | | c | dd | ddd | |
| 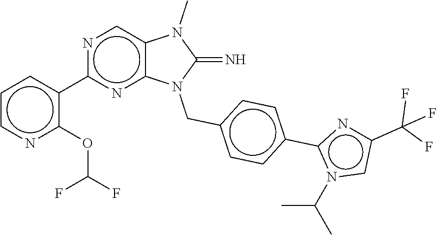 | T-051 | | c | cc | ddd | C |
| 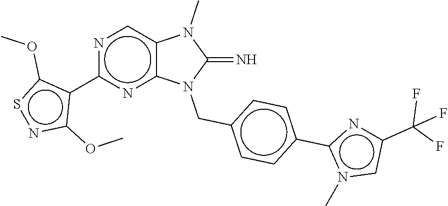 | T-052 | | b | bb | ccc | B |
| 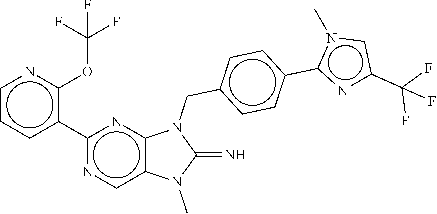 | T-053 | | a | aa | ddd | B |
| 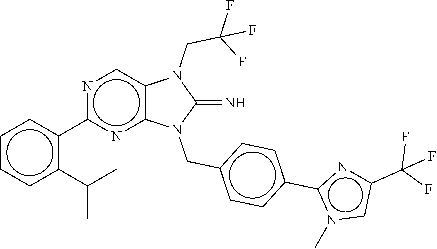 | T-054 | | b | aa | ddd | A |
| 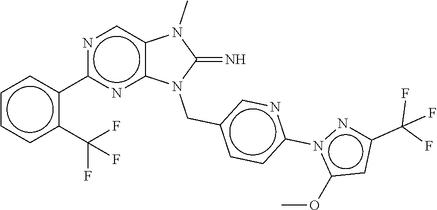 | T-055 | | c | cc | ddd | |
| 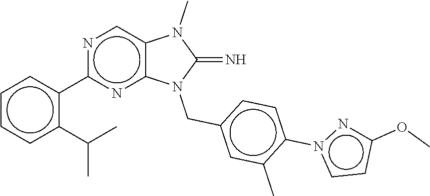 | T-056 | | c | cc | ccc | |
| 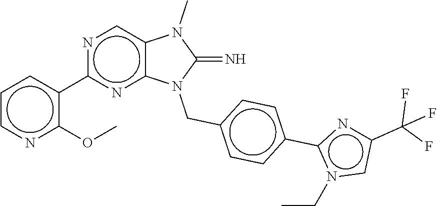 | T-057 | | c | cc | ddd | C |
| 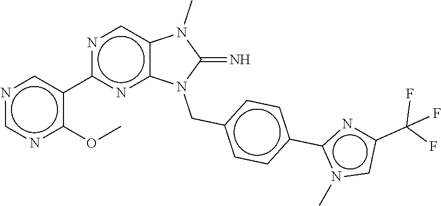 | T-058 | | c | dd | ddd | |
| 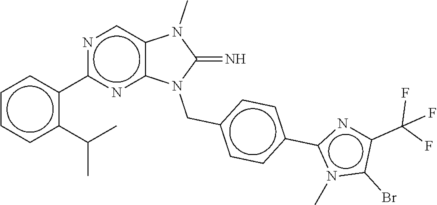 | T-059 | | a | aa | ccc | A |
| 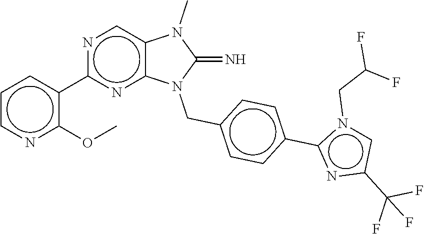 | T-060 | | c | dd | ddd | C |
| 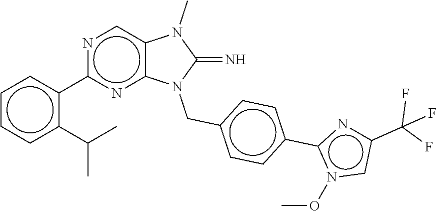 | T-061 | | a | bb | ccc | B |
| 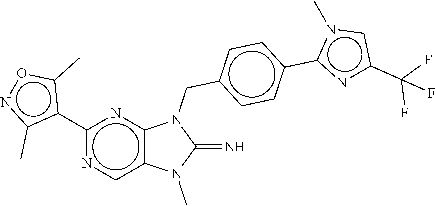 | T-062 | | c | cc | ddd | |
| 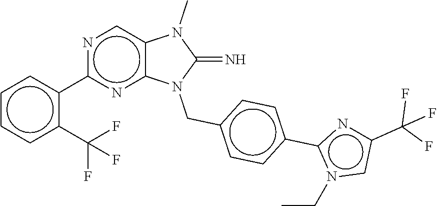 | T-063 | | b | bb | ddd | B |
| 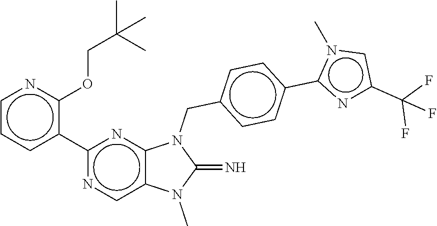 | T-064 | | c | cc | ddd | C |
| 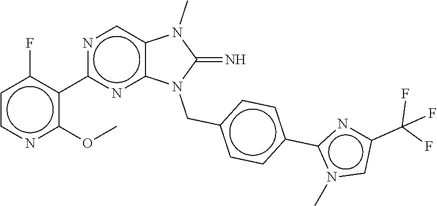 | T-065 | | b | bb | ddd | B |
| 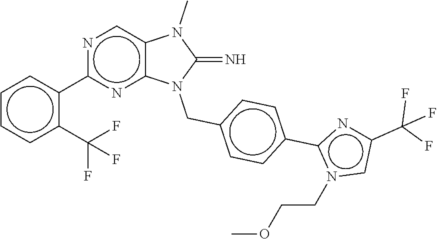 | T-066 | | c | cc | ddd | C |
| 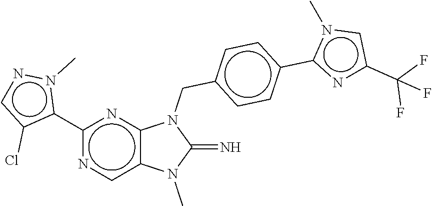 | T-067 | | b | cc | ddd | C |
| 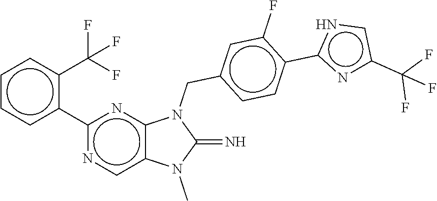 | T-068 | | c | cc | ccc | |
| 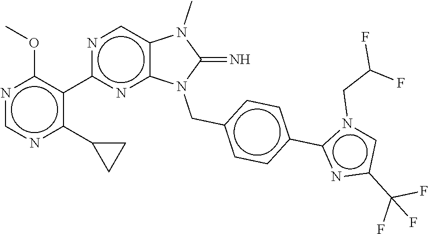 | T-069 | | b | bb | ddd | B |
| 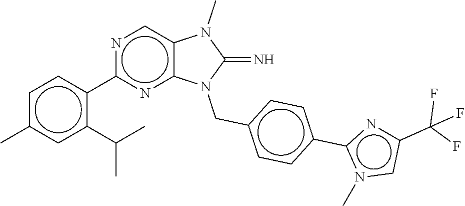 | T-070 | | b | cc | ccc | C |
| 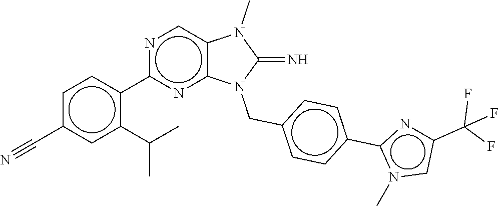 | T-071 | | b | bb | ccc | A |
| 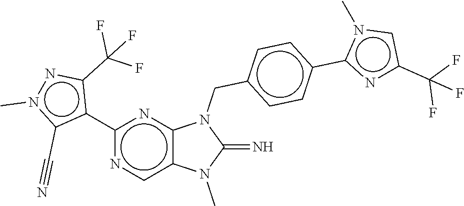 | T-072 | | c | cc | ccc | |
| 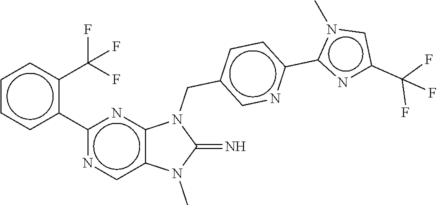 | T-073 | | c | cc | ddd | |
| 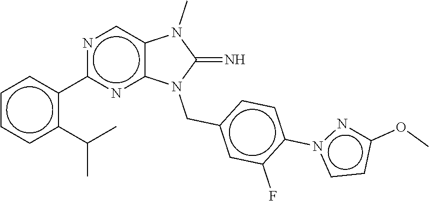 | T-074 | | c | cc | ccc | |
| 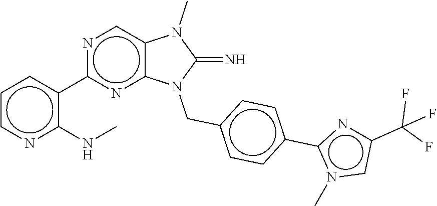 | T-075 | | c | cc | ccc | |
| 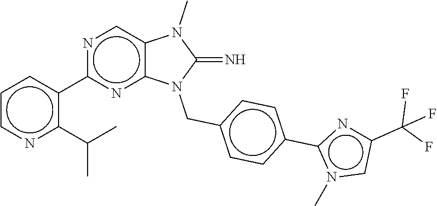 | T-076 | | b | cc | ddd | B |
| 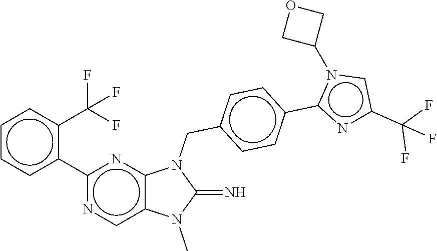 | T-077 | | c | cc | ddd | |
| 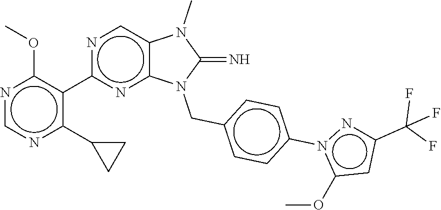 | T-078 | | a | aa | ddd | A |
| 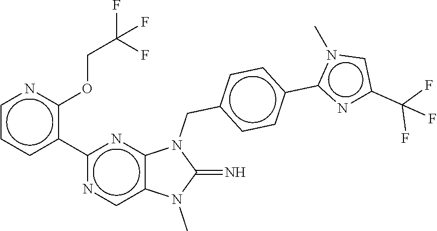 | T-079 | | c | cc | ddd | B |
| 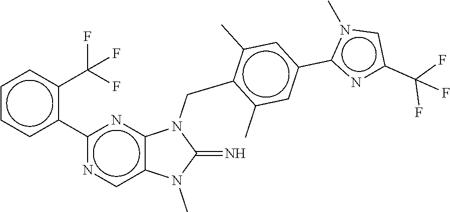 | T-080 | | c | cc | ccc | |
| 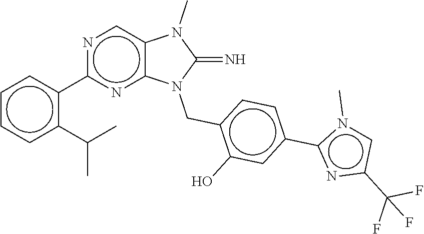 | T-081 | | a | bb | ddd | A |
| 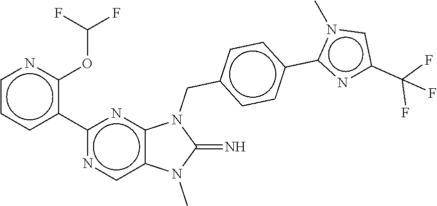 | T-082 | | a | aa | ddd | A |
| 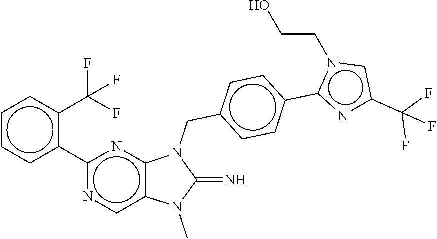 | T-083 | | c | cc | ddd | |
| 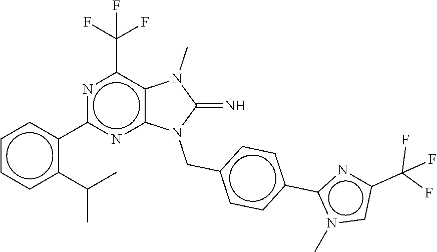 | T-084 | | a | bb | ccc | B |
| 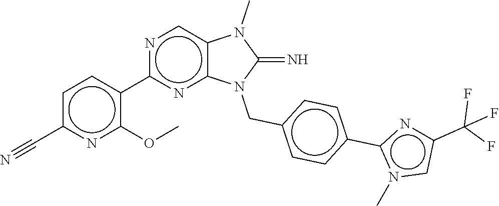 | T-085 | | c | cc | ccc | C |
| 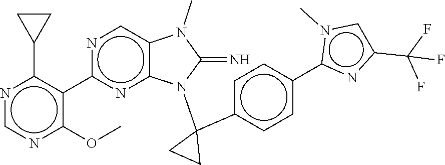 | T-086 | | | bb | ddd | B |
| 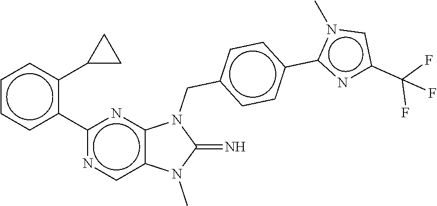 | T-087 | | a | aa | ccc | A |
| 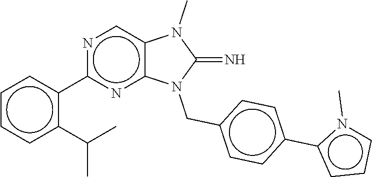 | T-088 | | c | | | |
| 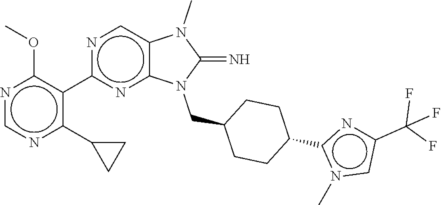 | T-089 | rel | c | cc | ddd | |
| 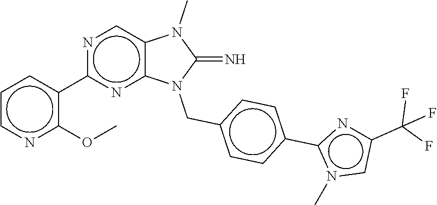 | T-090 | | c | cc | ddd | C |
| 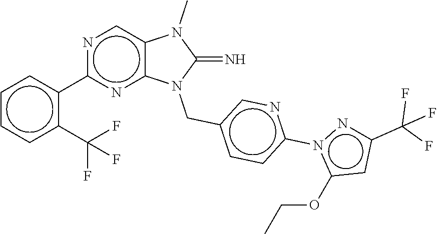 | T-091 | | c | cc | ddd | C |
| 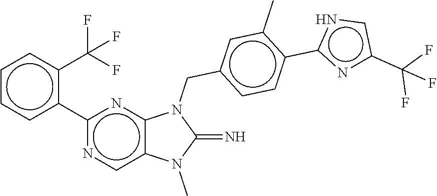 | T-092 | | b | cc | ccc | C |
| 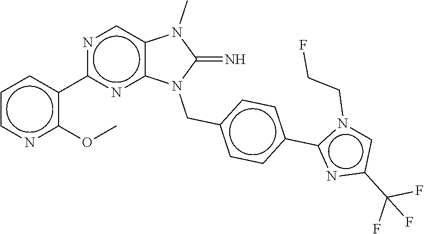 | T-093 | | c | cc | ddd | C |
| 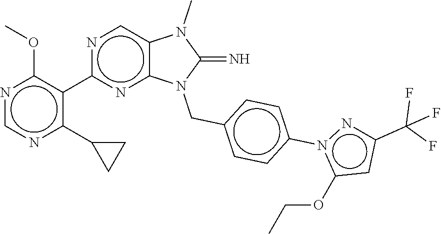 | T-094 | | a | aa | ddd | A |
| 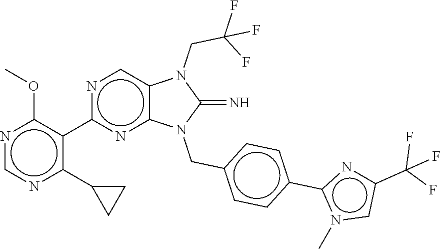 | T-095 | | b | aa | ddd | A |
| 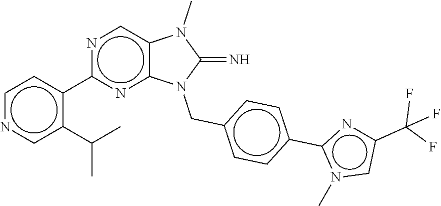 | T-096 | | c | cc | ddd | |
| 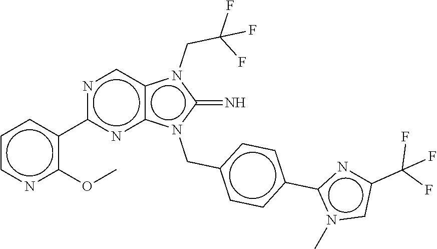 | T-097 | | b | bb | ddd | C |
| 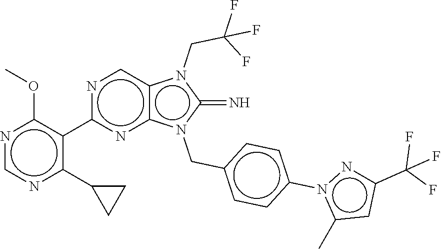 | T-098 | | b | aa | ddd | B |
| 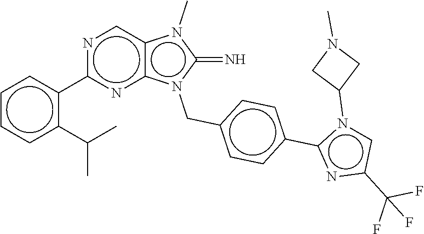 | T-099 | | c | aa | ddd | B |
| 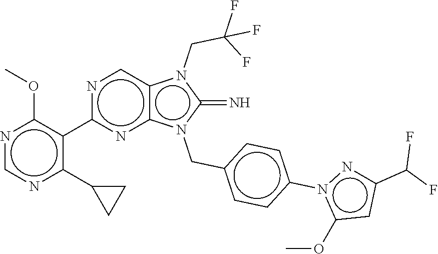 | T-100 | | c | bb | ddd | B |
| 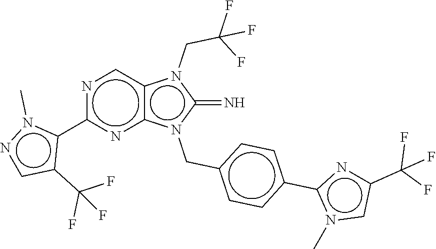 | T-101 | | a | bb | ccc | |
| 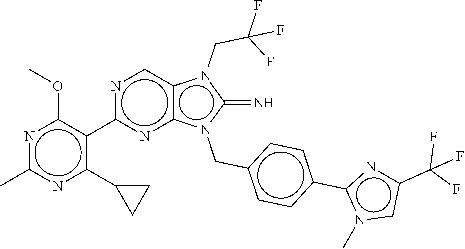 | T-102 | | c | bb | ddd | C |
| 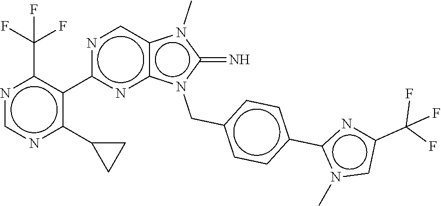 | T-103 | | b | bb | ddd | B |
| 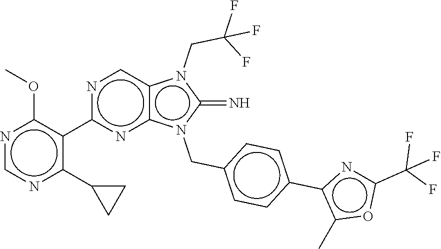 | T-104 | | c | bb | ddd | C |
| 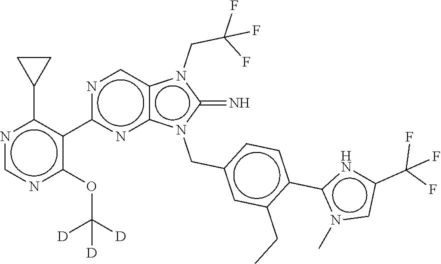 | T-105 | | c | cc | ddd | C |
| 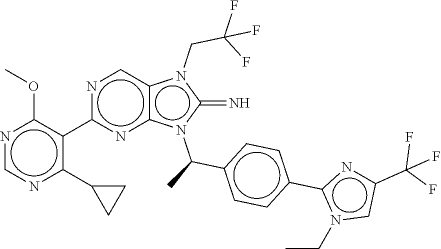 | T-106 | abs | a | aa | ddd | A |
| 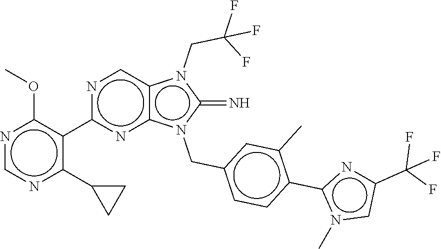 | T-107 | | c | bb | ddd | B |
| 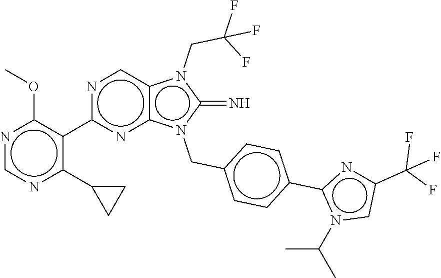 | T-108 | | b | aa | ddd | A |
| 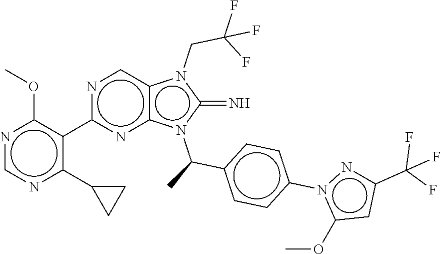 | T-109 | abs | a | aa | ddd | A |
| 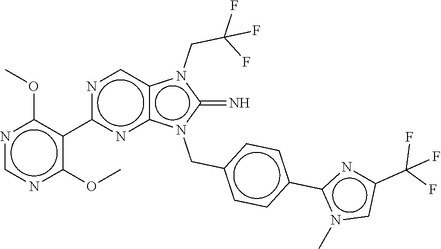 | T-110 | | b | bb | ddd | C |
| 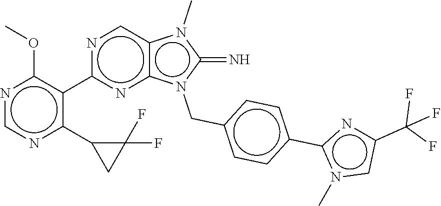 | T-111 | | b | aa | ddd | A |
| 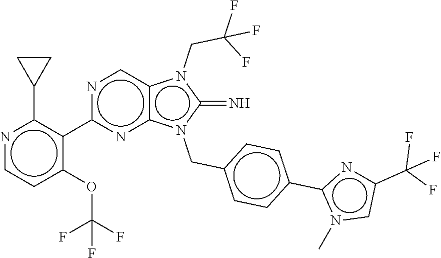 | T-112 | | b | aa | ddd | B |
| 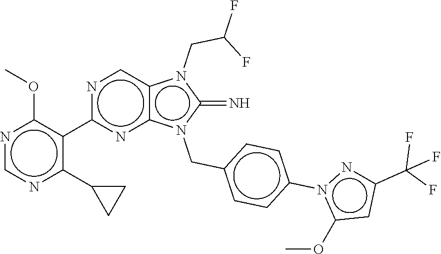 | T-113 | | a | aa | ddd | A |
| 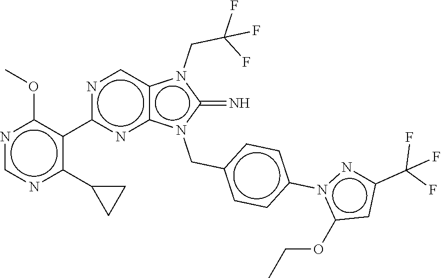 | T-114 | | b | aa | ddd | A |
| 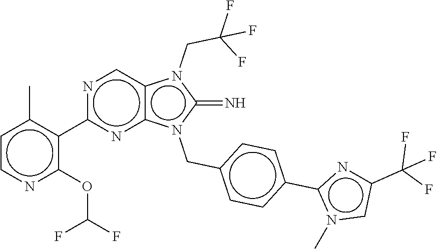 | T-115 | | b | bb | ccc | B |
| 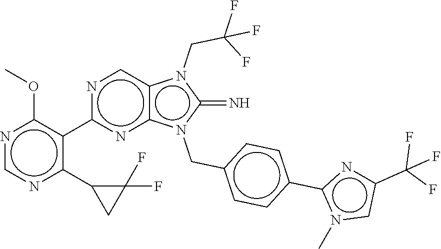 | T-116 | | c | aa | ddd | A |
| 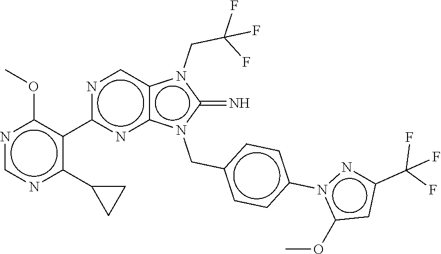 | T-117 | | b | aa | ddd | A |
| 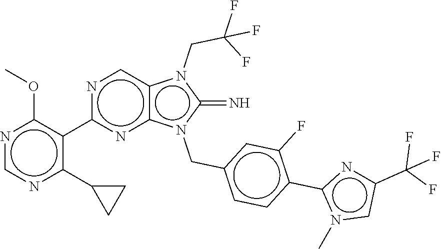 | T-118 | | c | aa | ddd | A |
| 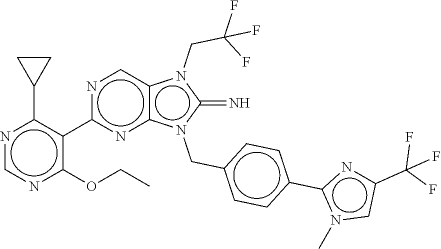 | T-119 | | c | bb | ddd | B |
| 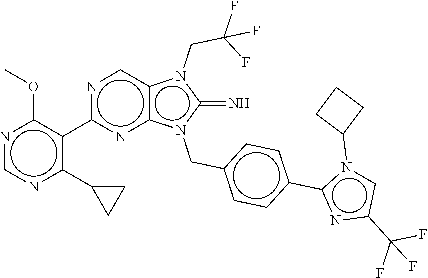 | T-120 | | a | aa | ddd | A |
| 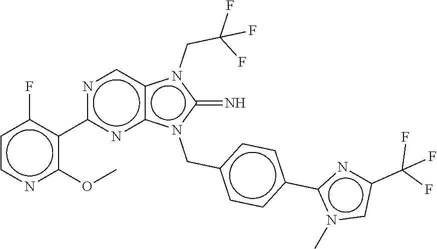 | T-121 | | b | bb | ddd | C |
| 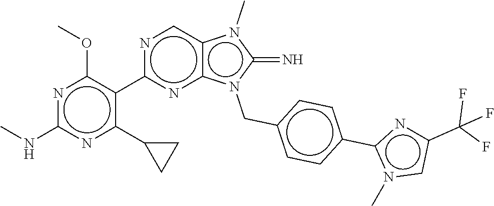 | T-122 | | c | bb | ddd | C |
| 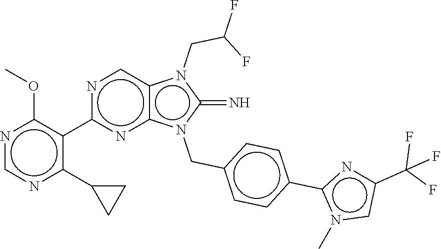 | T-123 | | b | aa | ddd | A |
| 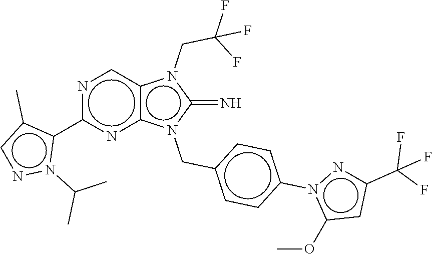 | T-124 | | a | aa | ddd | A |
| 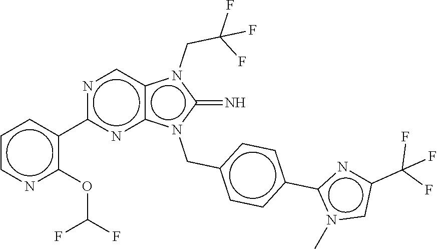 | T-125 | | b | bb | ccc | B |
| 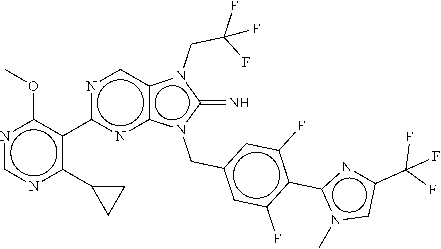 | T-126 | | b | aa | ddd | A |
| 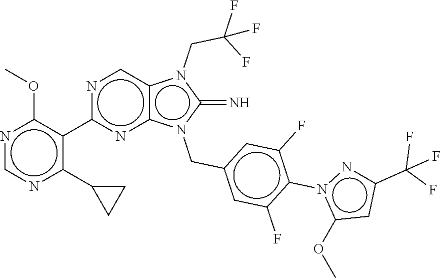 | T-127 | | b | aa | ddd | A |
| 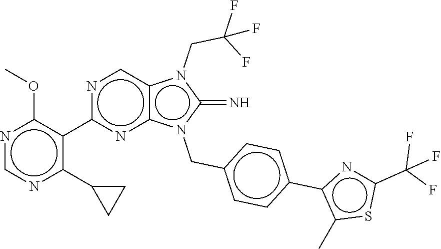 | T-128 | | a | aa | ccc | A |
| 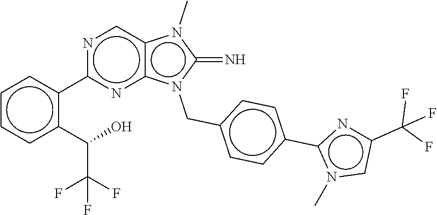 | T-129 | or | b | bb | ddd | B |
| 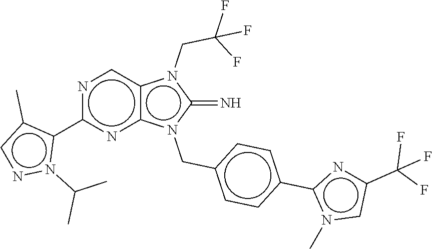 | T-130 | | a | aa | ddd | A |
| 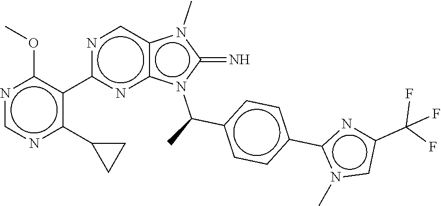 | T-131 | abs | b | aa | ddd | A |
| 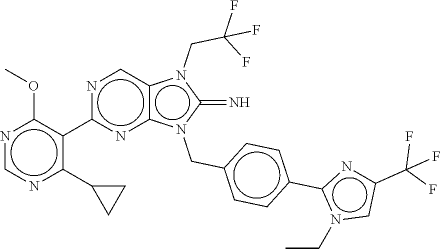 | T-132 | | b | aa | ddd | A |
| 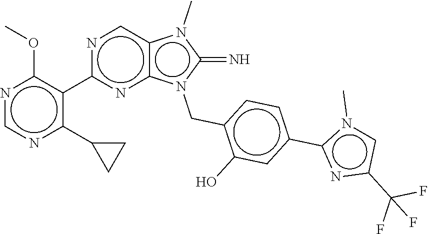 | T-133 | | b | aa | ddd | |
| 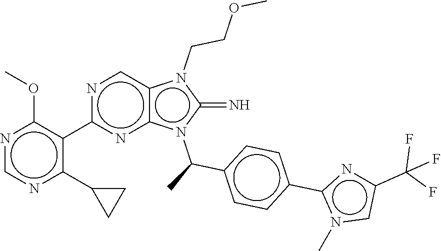 | T-134 | | c | bb | ddd | C |
| 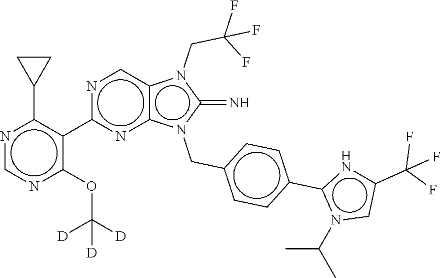 | T-135 | | b | aa | ddd | B |
| 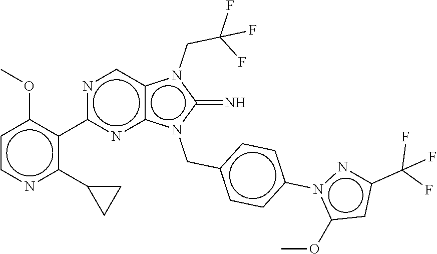 | T-136 | | b | bb | ddd | B |
| 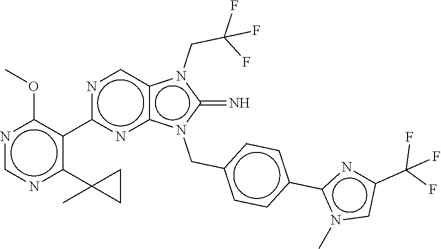 | T-137 | | c | bb | ddd | B |
| 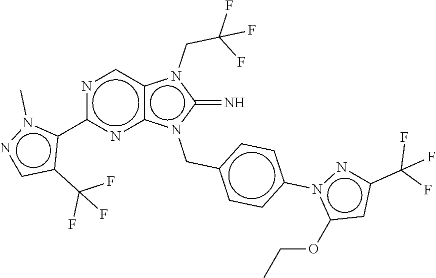 | T-138 | | b | bb | ccc | B |
| 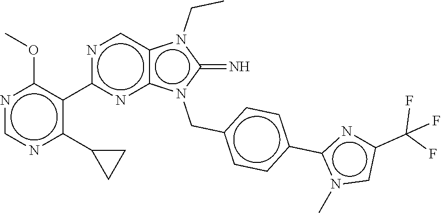 | T-139 | | b | aa | ddd | A |
| 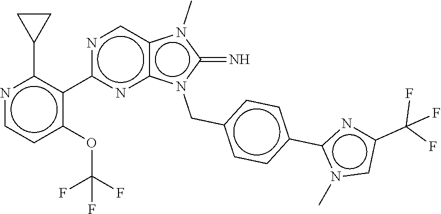 | T-140 | | b | aa | ddd | A |
| 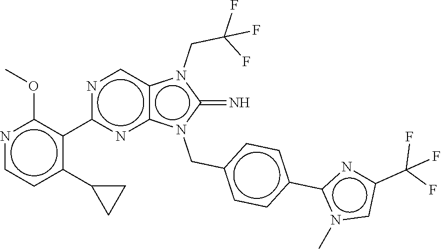 | T-141 | | c | bb | ddd | B |
| 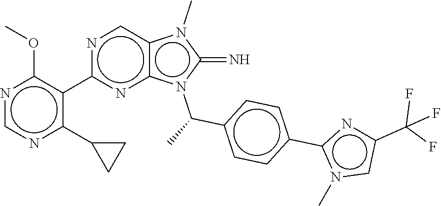 | T-142 | abs | c | bb | ddd | B |
| 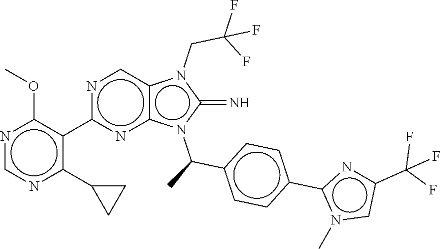 | T-143 | abs | b | aa | ddd | A |
| 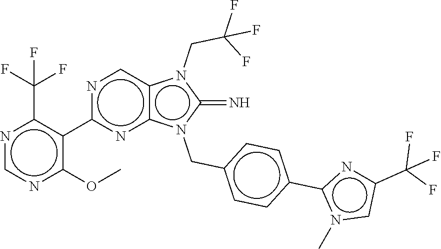 | T-144 | | b | bb | ddd | B |
| 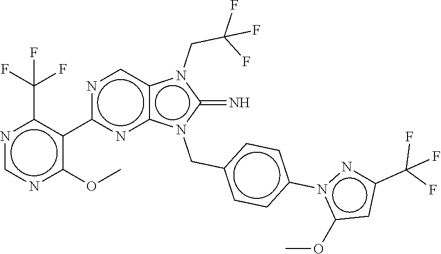 | T-145 | | b | bb | ccc | A |
| 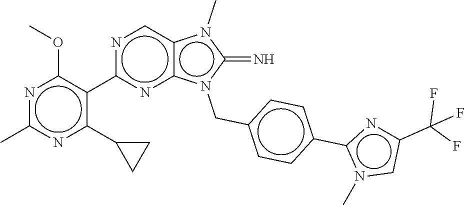 | T-146 | | b | aa | ddd | A |
| 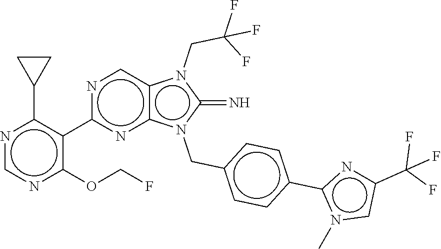 | T-147 | | b | bb | ddd | A |
| 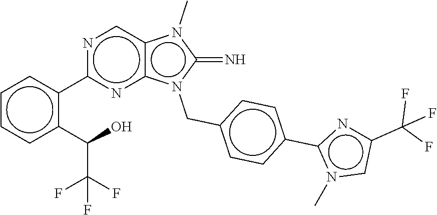 | T-148 | or | c | cc | ddd | B |

### ALTERNATIVE EMBODIMENTS

In an alternative embodiment, compounds described herein may also comprise one or more isotopic substitutions. For example, hydrogen may be ²H (D or deuterium) or ³H (T or tritium); carbon may be, for example, ¹³C or ¹⁴; oxygen may be, for example, ¹⁸O; nitrogen may be, for example, ¹⁵N, and the like. In other embodiments, a particular isotope (e.g., ³H, ¹³C ¹⁴C, ¹⁸O, or ¹⁵N) can represent at least 10%, at least 500, at least 10%, at least 15%0, at least 20%, at least 2500, at least 30%, at least 3500, at least 40%, at least 45%, at least 50%, at least 60%, at least 650%, at least 700%, at least 7500 at least 800%, at least 850%, at least 900%, at least 9500 at least 99%, or at least 99.9% of the total isotopic abundance of an element that occupies a specific site of the compound.

Pharmaceutical Compositions

In some embodiments, provided is a pharmaceutical composition comprising a pharmaceutically acceptable carrier and an effective amount of a compound described herein (e.g., a compound of Formula (I), (II) or a compound of Table 1), or a pharmaceutically acceptable salt, hydrate, solvate, prodrug, stereoisomer, or tautomer thereof.

The term “pharmaceutically acceptable carrier or adjuvant” refers to a carrier or adjuvant that may be administered to a patient, together with a compound provided herewith, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions provided herewith include, but are not limited to, ion exchangers, alumina, aluminum stearate, lecithin, self emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol and wool fat. Cyclodextrins such as α-, β-, and γ-cyclodextrin, or chemically modified derivatives such as hydroxyalkylcyclodextrins, including 2 and 3 hydroxypropyl-p-cyclodextrins, or other solubilized derivatives may also be advantageously used to enhance delivery of compounds of the formulae described herein.

When employed as pharmaceuticals, the compounds provided herein are typically administered in the form of a pharmaceutical composition. Such compositions can be prepared in a manner well known in the pharmaceutical art and comprise at least one active compound.

In some embodiments, with respect to the pharmaceutical composition, the carrier is a parenteral carrier, oral or topical carrier.

In some embodiments, provided is a compound described herein (e.g., a compound of Formula (I), (II) or a compound of Table 1), or a pharmaceutically acceptable salt, hydrate, solvate, prodrug, stereoisomer, or tautomer thereof) (or pharmaceutical composition thereof) for use as a pharmaceutical or a medicament (e.g., a medicament for the treatment of a disease or disorder associated with USP1 in a subject in need thereof). In some embodiments, the disease is a proliferating disease. In a further embodiment, the disease is cancer. In some embodiments, the cancer is breast cancer (e.g., triple negative breast cancer), ovarian cancer (e.g., platinum-resistant ovarian cancer, platinum-refractory ovarian cancer), prostate cancer, pancreatic cancer or lung cancer (e.g., non-small cell lung cancer (NSCLC)).

In some embodiments, provided is a compound described herein (e.g., a compound of Formula (I), (II) or a compound of Table 1), or a pharmaceutically acceptable salt, hydrate, solvate, prodrug, stereoisomer, or tautomer thereof) (or pharmaceutical composition thereof) for use in the treatment of a disease or disorder associated with USP1 in a subject in need thereof. In some embodiments, the disease is a proliferating disease. In a further embodiment, the disease is cancer. In some embodiments, the cancer is breast cancer (e.g., triple negative breast cancer), ovarian cancer (e.g., platinum-resistant ovarian cancer, platinum-refractory ovarian cancer), prostate cancer, pancreatic cancer or lung cancer (e.g., non-small cell lung cancer (NSCLC)).

In some embodiments, provided is a compound described herein (e.g., a compound of Formula (I), (II) or a compound of Table 1), or a pharmaceutically acceptable salt, hydrate, solvate, prodrug, stereoisomer, or tautomer thereof) (or pharmaceutical composition thereof) for use in the manufacturing of a medicament (e.g., a medicament for the treatment of an a disease or disorder associated with USP1 in a subject in need thereof). In some embodiments, the disease is a proliferating disease. In a further embodiment, the disease is cancer. In some embodiments, the cancer is breast cancer (e.g., triple negative breast cancer), ovarian cancer (e.g., platinum-resistant ovarian cancer, platinum-refractory ovarian cancer), prostate cancer, pancreatic cancer or lung cancer (e.g., non-small cell lung cancer (NSCLC)). Generally, the compounds provided herein are administered in a therapeutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herewith may be administered orally, parenterally, by inhalation spray, topically, rectally, nasally, buccally, vaginally or via an implanted reservoir, preferably by oral administration or administration by injection. The pharmaceutical compositions provided herewith may contain any conventional nontoxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like. The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The pharmaceutical compositions provided herewith may also be administered in the form of suppositories for rectal administration. These compositions can be prepared by mixing a compound provided herewith with a suitable non irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions provided herewith may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The above-described components for orally administrable, injectable or topically administrable, rectally administrable and nasally administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds disclosed herein can also be administered in sustained release forms or from sustained release drug delivery systems. A description of representative sustained release materials can be found in Remington's Pharmaceutical Sciences.

When the compositions provided herewith comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds provided herewith. Alternatively, those agents may be part of a single dosage form, mixed together with the compounds provided herewith in a single composition.

Also provided are pharmaceutically acceptable acid addition salt of a compound described herein (e.g., compound of Formula (I), (II) or a compound of Table 1).

The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect.

Typically, the pharmaceutical compositions provided herewith will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long term basis upon any recurrence of disease symptoms.

Methods of Treatment and Use

In some embodiments, the compounds described herein can be used to inhibit the activity of a USP1 protein. For example, in some embodiments, a method of inhibiting a USP1 protein comprises contacting the USP1 protein with a compound disclosed herein. The contacting can occur in vitro or in vivo.

In some embodiments, the compounds described herein can be used to treat a “USP1 protein mediated” disorder (e.g., a USP1 protein mediated cancer), a “USP1 associated” disorder (e.g., a USP1 associated cancer), or a disorder “associated with USP1” (e.g., a cancer associated with USP1). A “USP1 protein mediated”, “USP1 associated” disorder or a disorder “associated with USP1”, is any pathological condition in which a USP1 protein is known to play a role, including any cancers that require USP1 for cell proliferation and survival. In some embodiments, “USP1 protein mediated”, “USP1 associated” disorder or a disorder “associated with USP1” is a proliferative disease such as cancer. The method comprises administering to a patient in need of a treatment for a USP1 protein mediated disorder an effective amount of a compound of Formula (I), (II) or a compound of Table 1, or a pharmaceutically acceptable salt, hydrate, solvate, prodrug, stereoisomer, or tautomer thereof or a pharmaceutical composition comprising an effective amount of a compound of Formula (I), (II) or a compound of Table 1, or a pharmaceutically acceptable salt, hydrate, solvate, prodrug, stereoisomer, or tautomer thereof and a pharmaceutically acceptable excipient.

In some embodiments, provided is a method of treating a disease or disorder associated with modulation of USP1. The method comprises administering to a patient in need of a treatment for diseases or disorders associated with modulation of ubiquitin specific protease 1 (USP1) an effective amount of a compound of Formula (I), (II) or a compound of Table 1, or a pharmaceutically acceptable salt, hydrate, solvate, prodrug, stereoisomer, or tautomer thereof or a pharmaceutical composition comprising an effective amount of a compound of Formula (I), (II) or a compound of Table 1, or a pharmaceutically acceptable salt, hydrate, solvate, prodrug, stereoisomer, or tautomer thereof and a pharmaceutically acceptable excipient. In some embodiments the disease or disorder is cancer. In some embodiments, the compound or composition is administered in combination with a second therapeutic agent.

In some embodiments, provided is a method of treating or preventing cancer. The method comprises administering to a patient in need of a treatment for cancer an effective amount of a compound of Formula (I), (II) or a compound of Table 1 or a pharmaceutically acceptable salt, hydrate, solvate, prodrug, tautomer or stereoisomer thereof or a pharmaceutical composition comprising an effective amount of a compound of Formula (I), (II) or a compound of Table 1 or a pharmaceutically acceptable salt, hydrate, solvate, prodrug, tautomer or stereoisomer thereof and a pharmaceutically acceptable excipient.

In some embodiments, provided is a method of treating cancer. The method comprises administering to a patient in need thereof of a treatment for cancer an effective amount of a compound of Formula (I), (II) or a compound of Table 1 or a pharmaceutically acceptable salt, hydrate, solvate, prodrug, tautomer or stereoisomer thereof or a pharmaceutical composition comprising an effective amount of a compound of Formula (I), (II) or a compound of Table 1 or a pharmaceutically acceptable salt, hydrate, solvate, prodrug, tautomer or stereoisomer thereof and a pharmaceutically acceptable excipient.

In some embodiments, provided is a method of treating or preventing a disease or disorder associated with DNA damage. The method comprises administering to a patient in need of a treatment for diseases or disorders associated with DNA damage an effective amount of a compound of Formula (I), (II) or a compound of Table 1 or a pharmaceutically acceptable salt, hydrate, solvate, prodrug, tautomer or stereoisomer thereof or a pharmaceutical composition comprising an effective amount of a compound of Formula (I), (II) or a compound of Table 1 or a pharmaceutically acceptable salt, hydrate, solvate, prodrug, tautomer or stereoisomer thereof and a pharmaceutically acceptable excipient. In some embodiments the disease is cancer.

In some embodiments, provided is a method of treating a disease or disorder associated with DNA damage. The method comprises administering to a patient in need of a treatment for diseases or disorders associated with DNA damage an effective amount of a compound of Formula (I), (II) or a compound of Table 1 or a pharmaceutically acceptable salt, hydrate, solvate, prodrug, tautomer or stereoisomer thereof or a pharmaceutical composition comprising an effective amount of a compound of Formula (I), (II) or a compound of Table 1 or a pharmaceutically acceptable salt, hydrate, solvate, prodrug, tautomer or stereoisomer thereof and a pharmaceutically acceptable excipient.

In some embodiments, provided is a method of inhibiting, modulating or reducing DNA repair activity exercised by USP1. The method comprises administering to a patient in need thereof an effective amount of a compound of Formula (I), (II) or a compound of Table 1 or a pharmaceutically acceptable salt, hydrate, solvate, prodrug, tautomer or stereoisomer thereof or a pharmaceutical composition comprising an effective amount of a compound of Formula (I), (II) or a compound of Table 1 or a pharmaceutically acceptable salt, hydrate, solvate, prodrug, tautomer or stereoisomer thereof and a pharmaceutically acceptable excipient.

In some embodiments, provided is (a) a compound of Formula (I), (II) or a compound of Table 1, or a pharmaceutically acceptable salt, hydrate, solvate, prodrug, stereoisomer, or tautomer thereof or (b) a pharmaceutical composition comprising an effective amount of a compound of Formula (I), (II) or a compound of Table 1 or a pharmaceutically acceptable salt, hydrate, solvate, prodrug, tautomer or stereoisomer thereof and a pharmaceutically acceptable excipient, for use as a medicament.

In some embodiments, provided is (a) a compound of Formula (I), (II) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II) or a compound of Table 1 or a pharmaceutically acceptable salt, hydrate, solvate, prodrug, tautomer or stereoisomer thereof and a pharmaceutically acceptable excipient for use in the treatment or prevention of a disease associated with inhibiting USP1. In some embodiments the disease is cancer.

In some embodiments, provided is (a) a compound of Formula (I), (II) or a compound of Table 1 or a pharmaceutically acceptable salt, hydrate, solvate, prodrug, tautomer or stereoisomer thereof, or (b) a pharmaceutical composition comprising an effective amount of a compound of Formula (I), (II) or a compound of Table 1 or a pharmaceutically acceptable salt, hydrate, solvate, prodrug, tautomer or stereoisomer thereofand a pharmaceutically acceptable excipient for use in the treatment of a disease or disorder associated with inhibiting USP1.

In some embodiments, provided is (a) a compound of Formula (I), (II) or a compound of Table 1 or a pharmaceutically acceptable salt, hydrate, solvate, prodrug, tautomer or stereoisomer thereof, or (b) a pharmaceutical composition comprising an effective amount of a compound of Formula (I), (II) or a compound of Table 1 or a pharmaceutically acceptable salt, hydrate, solvate, prodrug, tautomer or stereoisomer thereof and a pharmaceutically acceptable excipient for use in the treatment or prevention of cancer.

In some embodiments, provided is (a) a compound of Formula (I), (II) or a compound of Table 1 or a pharmaceutically acceptable salt, hydrate, solvate, prodrug, tautomer or stereoisomer thereof, or (b) a pharmaceutical composition comprising an effective amount of a compound of Formula (I), (II) or a compound of Table 1 or a pharmaceutically acceptable salt, hydrate, solvate, prodrug, tautomer or stereoisomer thereof and a pharmaceutically acceptable excipient for use in the treatment of cancer.

In some embodiments, provided is (a) a compound of Formula (I), (II) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II) or a compound of Table 1, or a pharmaceutically acceptable salt, hydrate, solvate, prodrug, stereoisomer, or tautomer thereof and a pharmaceutically acceptable excipient for use in the treatment or prevention of a disease or disorder associated with DNA damage. In some embodiments the disease or disorder is cancer.

In some embodiments, provided is (a) a compound of Formula (I), (II) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II) or a compound of Table 1, or a pharmaceutically acceptable salt, hydrate, solvate, prodrug, stereoisomer, or tautomer thereof and a pharmaceutically acceptable excipient for use in the treatment of a disease or disorder associated with DNA damage. In some embodiments the disease or disorder is cancer.

In some embodiments, provided is (a) a compound of Formula (I), (II) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II) or a compound of Table 1, or a pharmaceutically acceptable salt, hydrate, solvate, prodrug, stereoisomer, or tautomer thereof and a pharmaceutically acceptable excipient for use in a method of inhibiting or reducing DNA repair activity modulated by USP1.

In some embodiments, provided is a compound of Formula (I), (II) or a compound of Table 1, or a pharmaceutical composition comprising a compound of Formula (I), (II) or a compound of Table 1 and a pharmaceutically acceptable carrier used for the treatment of cancers.

In some embodiments, provided is the use of (a) a compound of Formula (I), (II) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II) or a compound of Table 1, or a pharmaceutically acceptable salt, hydrate, solvate, prodrug, stereoisomer, or tautomer thereof and a pharmaceutically acceptable excipient in the manufacture of a medicament for treating or preventing a disease associated with inhibiting USP1. In some embodiments the disease or disorder is cancer.

In some embodiments, provided is the use of (a) a compound of Formula (I), (II) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II) or a compound of Table 1, or a pharmaceutically acceptable salt, hydrate, solvate, prodrug, stereoisomer, or tautomer thereof and a pharmaceutically acceptable excipient in the manufacture of a medicament for treating or preventing cancer.

In some embodiments, provided is the use of (a) a compound of Formula (I), (II) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II) or a compound of Table 1, or a pharmaceutically acceptable salt, hydrate, solvate, prodrug, stereoisomer, or tautomer thereof and a pharmaceutically acceptable excipient in the manufacture of a medicament for treating or preventing a disease or disorder associated with DNA damage. In some embodiments, the disease or disorder is cancer.

In some embodiments, provided is the use of (a) a compound of Formula (I), (II) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II) or a compound of Table 1, or a pharmaceutically acceptable salt, hydrate, solvate, prodrug, stereoisomer, or tautomer thereof and a pharmaceutically acceptable excipient in the manufacture of a medicament for treating a disease or disorder associated with DNA damage. In some embodiments, the disease or disorder is cancer.

In some embodiments, provided is the use of (a) a compound of Formula (I), (II) or a compound of Table 1, or a pharmaceutically acceptable salt, hydrate, solvate, prodrug, stereoisomer, or tautomer thereof, or (b) a pharmaceutical composition comprising an effective amount of a compound of Formula (I), (II) or a compound of Table 1, or a pharmaceutically acceptable salt, hydrate, solvate, prodrug, stereoisomer, or tautomer thereof and a pharmaceutically acceptable excipient in the manufacture of a medicament for inhibiting or reducing DNA repair activity modulated by USP1.

In some embodiments, provided is a pharmaceutical composition comprising a compound of Formula (I), (II) or a compound of Table 1, or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

In some embodiments, provided are methods of treating a disease or disorder associated with modulation of USP1 including, but not limited to, cancer comprising, administering to a patient suffering from at least one of said diseases or disorder (a) an effective amount (e.g., a therapeutically effective amount) of a compound of Formula (I), (II) or a compound of Table 1, or a pharmaceutically acceptable salt, hydrate, solvate, prodrug, stereoisomer, or tautomer thereof or (b) a pharmaceutical composition comprising an effective amount (e.g., a therapeutically effective amount) of a compound of Formula (I), (II) or a compound of Table 1, or a pharmaceutically acceptable salt, hydrate, solvate, prodrug, stereoisomer, or tautomer thereof and a pharmaceutically acceptable excipient; and one or more additional anti-cancer agent(s).

In some embodiments, the compound disclosed herein (e.g., a compound of Formula (I), (II) or a compound of Table 1, or a pharmaceutically acceptable salt, hydrate, solvate, prodrug, stereoisomer, or tautomer thereof) and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound disclosed herein and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In some embodiments, provided are kits that include one or more of the compounds disclosed herein (e.g., a compound of Formula (I), (II) or a compound of Table 1 or a pharmaceutically acceptable salt, hydrate, solvate, prodrug, stereoisomer or tautomer thereof) and a second therapeutic agent as disclosed herein are provided. Representative kits include (a) a compound disclosed herein or a pharmaceutically acceptable salt, hydrate, solvate, prodrug, stereoisomer, or tautomer thereof (e.g., a compound of Formula (I), (II) or a compound of Table 1 or a pharmaceutically acceptable salt, hydrate, solvate, prodrug, stereoisomer or tautomer thereof), (b) at least one other therapeutic agent, e.g., as indicated above, whereby such kit may comprise a package insert or other labeling including directions for administration.

In some embodiments of the methods and uses described herein, the cancer is selected from adrenocortical carcinoma, AIDS-related lymphoma, AIDS-related malignancies, anal cancer, cerebellar astrocytoma, extrahepatic bile duct cancer, bladder cancer, osteosarcoma/malignant fibrous histiocytoma, brain stem glioma, ependymoma, visual pathway and hypothalamic gliomas, breast cancer, bronchial adenomas/carcinoids, carcinoid tumors, gastrointestinal carcinoid tumors, carcinoma, adrenocortical, islet cell carcinoma, primary central nervous system lymphoma, cerebellar astrocytoma, cervical cancer, chronic lymphocytic leukemia, chronic myelogenous leukemia, clear cell sarcoma of tendon sheaths, colon cancer, colorectal cancer, cutaneous t-cell lymphoma, endometrial cancer, ependymoma, esophageal cancer, Ewing's sarcoma/family of tumors, extracranial germ cell tumors, extragonadal germ cell tumors, extrahepatic bile duct cancer, eye cancers, including intraocular melanoma, and retinoblastoma, gallbladder cancer, gastrointestinal carcinoid tumor, ovarian germ cell tumor, gestational trophoblastic tumor, hairy cell leukemia, head and neck cancer, Hodgkin's disease, hypopharyngeal cancer, Kaposi's sarcoma, laryngeal cancer, acute lymphoblastic leukemia, acute myeloid leukemia, liver cancer, non-small cell lung cancer, small cell lung cancer, non-Hodgkin's lymphoma, Waldenstrom's macroglobulinemia, malignant mesothelioma, malignant thymoma, medulloblastoma, melanoma, merkel cell carcinoma, metastatic squamous neck cancer with occult primary, multiple endocrine neoplasia syndrome, multiple myeloma/plasma cell neoplasm, mycosis fungoides, myelodysplastic syndrome, chronic myelogenous leukemia, myeloid leukemia, multiple myeloma, myeloproliferative disorders, nasal cavity and paranasal sinus cancer, nasopharyngeal cancer, neuroblastoma, oral cancer, oral cavity and lip cancer, oropharyngeal cancer, osteosarcoma/malignant fibrous histiocytoma of bone, ovarian cancer, ovarian low malignant potential tumor, pancreatic cancer, paranasal sinus and nasal cavity cancer, parathyroid cancer, penile cancer, pheochromocytoma, pituitary tumor, pleuropulmonary blastoma, prostate cancer, rectal cancer, renal cell (kidney) cancer, transitional cell cancer (e.g., renal pelvis and ureter), retinoblastoma, rhabdomyosarcoma, salivary gland cancer, malignant fibrous histiocytoma of bone, soft tissue sarcoma, sezary syndrome, skin cancer, small intestine cancer, stomach (gastric) cancer, supratentorial primitive neuroectodennal and pineal tumors, cutaneous t-cell lymphoma, testicular cancer, malignant thymoma, thyroid cancer, gestational trophoblastic tumor, urethral cancer, uterine sarcoma, vaginal cancer, vulvar cancer, and Wilms' tumor. In other embodiments, the cancer is a non-small cell lung cancer.

In any of the embodiments, the cancer can be any cancer in any organ, for example, a cancer is selected from the group consisting of glioma, thyroid carcinoma, breast carcinoma, small-cell lung carcinoma, non-small-cell carcinoma, gastric carcinoma, colon carcinoma, gastrointestinal stromal carcinoma, pancreatic carcinoma, bile duct carcinoma, CNS carcinoma, ovarian carcinoma, endometrial carcinoma, prostate carcinoma, renal carcinoma, anaplastic large-cell lymphoma, leukemia, multiple myeloma, mesothelioma, and melanoma, and combinations thereof.

In some embodiments, the cancer to be treated with a compound disclosed herein is selected from the group consisting of bone cancer, including osteosarcoma and chondrosarcoma; brain cancer, including glioma, glioblastoma, astrocytoma, medulloblastoma, and meningioma; soft ti ssue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer.

In other embodiments, the cancer is selected from liposarcoma, neuroblastoma, glioblastoma, bladder cancer, adrenocortical cancer, multiple myeloma, colorectal cancer, non-small cell lung cancer, Human Papilloma Virus-associated cervical, oropharyngeal, penis, anal, thyroid or vaginal cancer or Epstein-Barr Virus-associated nasopharyngeal carcinoma, gastric cancer, rectal cancer, thyroid cancer, Hodgkin lymphoma and diffuse large B-cell lymphoma.

In some embodiments, the cancer is selected from breast cancer (e.g., triple negative breast cancer (TNBC)), ovarian cancer (e.g., platinum-resistant ovarian cancer, platinum-refractory ovarian cancer), prostate cancer, pancreatic cancer and lung cancer (e.g., non-small cell lung cancer (NSCLC)). In some embodiments, the cancer is selected from breast cancer (e.g., triple negative breast cancer (TNBC)), ovarian cancer (e.g., platinum-resistant ovarian cancer, platinum-refractory ovarian cancer), prostate cancer and lung cancer (e.g., non-small cell lung cancer (NSCLC)). In some embodiments, the cancer is breast cancer. In some embodiments the cancer is triple negative breast cancer (TNBC). In some embodiments the cancer is prostate cancer. In some embodiments the cancer is lung cancer. In some embodiments the cancer is non-small cell lung cancer (NSCLC).

In certain embodiments of the methods described herein, the cancer is a dedifferentiated ID-driven cancer. In other embodiments, the cancer is a cancer that is sensitive to USP1 inhibition. In yet other embodiments, the cancer is a cancer that is sensitive to USP1 inhibition due to DNA damage pathway deficiency.

In some embodiments of the methods and uses described herein, the cancer is selected from the group consisting of a hematological cancer, a lymphatic cancer, and a DNA damage repair pathway deficient cancer.

In some embodiments, a compound disclosed herein is used to treat a cancer, wherein the cancer is a homologous recombination deficient cancer. In some embodiments, a compound disclosed herein is used to treat a cancer that does not have a defect in the homologous recombination pathway.

In some embodiments, the cancer is a DNA damage repair pathway deficient cancer. In some embodiments, the DNA damage repair pathway deficient cancer is selected from the group consisting of lung cancer, non-small cell lung cancer (NSCLC), colon cancer, bladder cancer, osteosarcoma, ovarian cancer (e.g., platinum-resistant ovarian cancer, platinum-refractory ovarian cancer), and breast cancer (e.g., triple negative breast cancer (TNBC)). In some embodiments, the cancer is non-small cell lung cancer (NSCLC). In some embodiments, the cancer is colon cancer. In some embodiments, the cancer is bladder cancer. In some embodiments, the cancer is ovarian cancer or breast cancer. In some embodiments, the cancer is ovarian cancer. In some embodiments, the cancer is platinum-resistant ovarian cancer. In some emobodiments, the cancer is platinum-refractory ovarian cancer. In some embodiments, the cancer is breast cancer. In some embodiments, the cancer is triple negative breast cancer.

In some embodiments, the cancer is a HRR (homologous recombination repair) gene mutant cancer. In some embodiments, the cancer is a HRR (homologous recombination repair) gene mutant cancer selected from the group consisting of ATM, BARD1, BRCA1, BRCA2, BRIP1, CDK12, CHEK1, CHEK2, FANCL, PALB2, PPP2R2A, RAD51B, RAD51C, RAD51D, or RAD54L mutant cancer. In some embodiments, the cancer is an ATM mutant cancer. In some embodiments, the cancer is an BARD1 mutant cancer. In some embodiments, the cancer is an BRCA1 mutant cancer. In some embodiments, the cancer is an BRCA2 mutant cancer. In some embodiments, the cancer is an BRIP1 mutant cancer. In some embodiments, the cancer is an CDK12 mutant cancer. In some embodiments, the cancer is an CHEK1 mutant cancer. In some embodiments, the cancer is an CHEK2 mutant cancer. In some embodiments, the cancer is an FANCL mutant cancer. In some embodiments, the cancer is an PALB2 mutant cancer. In some embodiments, the cancer is an PPP2R2A mutant cancer. In some embodiments, the cancer is an RAD51B mutant cancer. In some embodiments, the cancer is an RAD51C mutant cancer. In some embodiments, the cancer is an RAD51D mutant cancer. In some embodiments, the cancer is an RAD54L mutant cancer.

In some embodiments, the cancer is a BRCA1 mutant cancer. In some embodiments, the BRCA1 mutation is a germline mutation. In some embodiments, the BRCA1 mutation is a somatic mutation. In some embodiments, the BRCA1 mutation leads to BRCA1 deficiency. In some embodiments, the cancer is a BRCA2 mutant cancer. In some embodiments, the BRCA2 mutation is a germline mutation. In some embodiments, the BRCA2 mutation is a somatic mutation. In some embodiments, the BRCA2 mutation leads to BRCA2 deficiency. In some embodiments, the cancer is a BRCA1 mutant cancer and a BRCA2 mutant cancer. In some embodiments, the cancer is a BRCA1 deficient cancer. In some embodiments, the cancer is a BRCA2 deficient cancer. In some embodiments, the cancer is a BRCA1 deficient cancer and a BRCA2 deficient cancer. In some embodiments, the cancer is not a BRCA1 mutant cancer or a BRCA2 mutant cancer. In some embodiments, the cancer is a BRCA1 deficient cancer and a BRCA2 mutant cancer. In some embodiments, the BRCA1 or BRCA2 mutant or BRCA1 or BRCA2 deficient cancer is selected from non-small cell lung cancer (NSCLC), osteosarcoma, prostate cancer, pancreatic cancer, ovarian cancer, and breast cancer. In some embodiments, the BRCA1 mutant, BRCA2 mutant, BRCA1 deficient or BRCA 2 deficient cancer as described herein is ovarian cancer, breast cancer, prostate cancer or pancreatic cancer. In some embodiments, the cancer is ovarian cancer. In some embodiments, the cancer is platinum-resistant ovarian cancer. In some emobodiments, the cancer is platinum-refractory ovarian cancer. In some embodiments, the cancer is breast cancer. In some embodiments, the cancer is a triple negative breast cancer. In some embodiments, the cancer is prostate cancer. In some embodiments, the cancer is homologous recombination deficient. Homologous recombination deficiency can be measured by BRCA1/2 mutation, or genomic instability (positive homologous recombination deficiency (HRD) score) without BRCA1/2 mutations.

In some embodiments, the cancer is a Poly (ADP-ribose) polymerase (“PARP”) inhibitor refractory or resistant cancer. In some embodiments, the cancer is a PARP inhibitor resistant or refractory BRCA1, BRCA2, or BRCA1 and BRCA2 mutant cancer. In some embodiments, the cancer is a PARP inhibitor resistant or refractory BRCA1, BRCA2, or BRCA1 and BRCA2-deficient cancer. In some embodiments, the PARP inhibitor refractory or resistant cancer is selected from the cancers described herein. In some embodiments, the PARP inhibitor refractory or resistant cancer is selected from breast cancer (e.g., triple negative breast cancer (TNBC), ovarian cancer (e.g., platinum-resistant ovarian cancer, platinum-refractory ovarian cancer), pancreatic cancer and prostate cancer).

In some embodiments, the cancer has a mutation in the gene encoding ataxia telangiectasia mutated (ATM) protein kinase or loss of ATM protein expression. In some embodiments, the cancer to be treated with a compound disclosed herein is a cancer (e.g., a cancer selected from the cancers described herein) that comprises cancer cells with a loss of function mutation in a gene encoding ATM. In some embodiments the ATM mutation is a germline mutation. In some embodiments the ATM mutation is a somatic mutation. In some embodiments, the cancer is not an ATM mutant cancer. In some embodiments the cancer is an ATM-deficient cancer. In some embodiments, the ATM-deficient cancer is selected from breast cancer (e.g., triple negative breast cancer (TNBC)), ovarian cancer (e.g., platinum-resistant ovarian cancer, platinum-refractory ovarian cancer), colorectal cancer, stomach cancer, endometrial cancer, urothelial cancer, cervical cancer, melanoma, esophageal cancer, head and neck cancer, mantle cell lymphoma, sarcoma, prostate cancer, pancreatic cancer, and lung cancer (e.g., non-small cell lung cancer (NSCLC)).

In some embodiments, the cancer comprises cancer cells with elevated levels of translesion synthesis. This includes cancers that exhibit elevated PCNA monoubiquitination, with or without elevated levels of RAD18 and/or UBE2K. In some embodiments, the elevated levels of RAD18 and/or UBE2K are elevated RAD18 and/or UBE2K protein levels. In some embodiments, the elevated levels of RAD18 and/or UBE2K are elevated RAD18 and/or UBE2K mRNA levels. In some embodiments, elevated levels of RAD18 and/or UBE2K (e.g., RAD18 and/or UBE2K protein and/or RAD18 and/or UBE2K mRNA) have been detected (e.g., in a cancer sample obtained from the subject) prior to the administration. Elevated translesion synthesis can also be measured by PCNA monoubiquitination without elevated RAD18 and/or UBE2K levels. In some embodiments, a subject's cancer has been tested for RAD18 and/or UBE2K levels protein or mRNA, or PCNA monoubiquitination prior to beginning treatment with a USP1 inhibitor. In some embodiments, the cancer is a breast cancer (e.g., triple negative breast cancer), an ovarian cancer, a lung cancer (e.g., non-small cell lung cancer (NSCLC)), or a prostate cancer.

In some embodiments, the cancer is a BRCA1 and/or BRCA2 mutant cancer, wherein the cancer comprises cells with increased translesion synthesis, as exemplified by elevated PCNA monoubiquitination with or without elevated RAD18 and/or UBE2K levels. In some embodiments, the cancer is a breast cancer (e.g., triple negative breast cancer), an ovarian cancer or a prostate cancer that is a BRCA1 and/or BRCA2 mutant cancer.

In some embodiments, the cancer is selected from the group consisting of bone cancer, including osteosarcoma and chondrosarcoma; brain cancer, including glioma, glioblastoma, astrocytoma, medulloblastoma, and meningioma; soft tissue cancer, including rhabdoid and sarcoma; kidney cancer; bladder cancer; skin cancer, including melanoma; and lung cancer, including non-small cell lung cancer; colon cancer, uterine cancer; nervous system cancer; head and neck cancer; pancreatic cancer; and cervical cancer.

Combination Therapies

In some embodiments, the compounds of the disclosure are administered in therapeutically effective amounts in a combination therapy with one or more therapeutic agents (pharmaceutical combinations) or modalities, e.g., non-drug therapies. For example, synergistic effects can occur with other anti-proliferative, anti-cancer, immunomodulatory or anti-inflammatory substances. Where the compounds of the disclosure are administered in conjunction with other therapies, dosages of the co-administered compounds will vary depending on the type of co-drug employed, on the specific drug employed, on the condition being treated and so forth.

In some embodiments, provided are methods of treatment of a disease or disorder associated with the USP1 with a compound of Formula (I), (II) or a compound of Table 1 or a pharmaceutically acceptable salt, hydrate, solvate, prodrug, stereoisomer or tautomer thereof) in combination with a second therapeutic agent. In some embodiments, provided are methods of treatment of a disease or disorder associated with USP1 with a compound of Formula (I), (II) or a compound of Table 1 or pharmaceutically acceptable salts thereof in combination with a second therapeutic agent and a third therapeutic agent. In some embodiments, provided are methods of treatment of a disease or disorder associated with the USP1 with a compound of Formula (I), (II) or a compound of Table 1, or pharmaceutically acceptable salts thereof) in combination with a second therapeutic agent, a third therapeutic agent, and a fourth therapeutic agent.

The term “Combination” refers to either a fixed combination in one dosage unit form, or a combined administration where a compound disclosed herein (e.g., a compound of Formula (I), (II) or a compound of Table 1 or a pharmaceutically acceptable salt, hydrate, solvate, prodrug, stereoisomer or tautomer thereof) and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., a compound disclosed herein (e.g., a compound of Formula (I), (II) or a compound of Table 1 or a pharmaceutically acceptable salt, hydrate, solvate, prodrug, stereoisomer or tautomer thereof) and a combination partner, are both administered to a patient simultaneously in the form of a single entity or dosage. The term “non-fixed combination” means that the therapeutic agents, e.g., a compound disclosed herein (e.g., a compound of Formula (I), (II) or a compound of Table 1 or a pharmaceutically acceptable salt, hydrate, solvate, prodrug, stereoisomer or tautomer thereof) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times.

In certain embodiments, compounds disclosed herein are combined with other therapeutic agents, including, but not limited to, other anti-cancer agents, anti-allergic agents, anti-nausea agents (or anti-emetics), pain relievers, cytoprotective agents, and combinations thereof.

In some embodiments, provided is a method of treating a disease or disorder associated with USP1 (e.g., cancer) comprising administering or coadministering, in any order, to a patient in need thereof a compound disclosed herein (e.g., a compound of Formula (I), (II) or a compound of Table 1 or pharmaceutically acceptable salts thereof) and a general chemotherapeutic agent selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, provided is a method of treating a disease or disorder associated with USP1 (e.g., cancer) comprising administering or coadministering, in any order, to a patient in need thereof a compound disclosed herein (e.g., a compound of Formula (I), (II) or a compound of Table 1 or pharmaceutically acceptable salts thereof) and an EGFR-inhibitor (e.g., cetuximab, panitumimab, erlotinib, gefitinib and EGFRi NOS). In some embodiments, provided is a method of treating a disease or disorder associated with USP1 (e.g., cancer) comprising administering or coadministering, in any order, to a patient in need thereof a compound disclosed herein (e.g., a compound of Formula (I), (II) or a compound of Table 1 or pharmaceutically acceptable salts thereof) and a MAPK-pathway inhibitor (e.g., BRAFi, panRAFi, MEKi, ERKi) In some embodiments, provided is a method of treating a disease or disorder associated with USP1 (e.g., cancer) comprising administering or coadministering, in any order, to a patient in need thereof a compound disclosed herein (e.g., a compound of Formula (I), (II) or a compound of Table 1 or pharmaceutically acceptable salts thereof) and a PI3K-mTOR pathway inhibitor (e.g., alpha-specific PI3Ki, pan-class I PI3Ki and mTOR/PI3Ki, particularly everolimus and analogues thereof).

In some embodiments, provided is a method of enhancing the chemotherapeutic treatment of cancer in a mammal undergoing treatment with an anti-cancer agent, which method comprises co-administering to the mammal an effective amount of a compound disclosed herein. In certain embodiments, provided is a method of treating a disease or disorder associated with USP1 (e.g., cancer) comprising administering or coadministering, in any order, to a patient in need thereof a compound disclosed herein (e.g., a compound of Formula (I), (II) or a compound of Table 1 or pharmaceutically acceptable salts thereof) and a DNA damaging agent (e.g., actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, tenyposide, triethylenethiophosphoramide and etoposide). In a preferred embodiment, the DNA damaging agent is cisplatin. In some embodiments, the DNA damaging agent is radiation or a biotherapeutic agent (e.g., an antibody).

In some embodiments, the anti-cancer agent is selected from reversible DNA binders (e.g., topotecan hydrochloride, irinotecan (CPT1 1—Camptosar), rubitecan, exatecan, nalidixic acid, TAS-103, etoposide, acridines (e.g., amsacrine, aminocrine), actinomycins (e.g., actinomycin D), anthracyclines (e.g., doxorubicin, daunorubicin), benzophenainse, XR 1 1576/MLN 576, benzopyridoindoles, Mitoxantrone, AQ4, Etoposide, Teniposide, epipodophyllotoxins, and bisintercalating agents such as triostin A and echinomycin), DNA alkylators (e.g., sulfur mustard, the nitrogen mustards (e.g., mechlorethamine), chlorambucil, melphalan, ethyleneimines (e.g., triethylenemelamine, carboquone, diaziquone), methyl methanesulfonate, busulfan, CC-1065, duocarmycins (e.g., duocarmycin A, duocarmycin SA), metabolically activated alkylating agents such as nitrosoureas (e.g., carmustine, lomustine, (2-chloroethyl)nitrosoureas), triazine antitumor drugs such as triazenoimidazole (e.g., dacarbazine), mitomycin C and leinamycin), DNA strand breakers (e.g., doxorubicin and daunorubicin (which are also reversible DNA binders), other anthracyclines, bleomycins, tirapazamine, enediyne antitumor antibiotics such as neocarzinostatin, esperamicins, calicheamicins, dynemicin A, hedarcidin, C-1027, N1999A2, esperamicins and zinostatin), and disruptors of DNA replication (e.g., 5-fluorodeoxyuridine).

In certain embodiments, the DNA damaging agent is radiation (e.g., radiation that induces a DNA cross-linking in a cell when applied to the cell, (e.g., ionizing radiation and ultraviolet (UV) radiation)). Ionizing radiation consists of subatomic particles or electromagnetic waves that are sufficiently energetic to cause ionization by detaching electrons from atoms or molecules. Ionization depends on the energy of the impinging individual particles or waves. In general, ionizing particles or photons with energies above a few electron volts can be ionizing. Non-limiting examples of ionizing particles are alpha particles, beta particles, and neutrons. The ability of photons to ionize a atom or molecule depends on its frequency. Short-wavelength radiation such as high frequency ultraviolet, x-rays, and gamma rays, is ionizing. Ionizing radiation comes from radioactive materials, x-ray tubes, and particle accelerators.

In certain embodiments, the anticancer agent targets a USP1 independent mechanism of DNA repair. Non-limiting examples of suitable DNA repair inhibitors are poly (ADP-ribose) polymerase (PARP) inhibitors, DNA-dependent protein kinase (DNA-PK) inhibitors, ataxia telangiectasia and Rad3-related protein (ATR) inhibitors, ataxia-telangiectasia mutated (ATM) inhibitors, checkpoint kinase 1 (CHK1) inhibitors, checkpoint kinase 2 (CHK2) inhibitors, and Weel inhibitors. It has been reported that BRCA1/2 status predicts the efficacy of PARP inhibitors in the clinic (Audeh et al. Lancet (2010) 376 (9737), 245-51). In general, BRCA1/2 mutant cancers have increased sensitivity to USP1 inhibitors. Accordingly, in some embodiments, a In certain embodiments, provided is a method of treating a disease or disorder associated with USP1 (e.g., cancer) comprising administering or coadministering, in any order, to a patient in need thereof a compound disclosed herein (e.g., a compound of Formula (I), (II) or a compound of Table 1 or pharmaceutically acceptable salts thereof) and a PARP inhibitor (e.g., olaparib, rucaparib, niraparib, talazoparib, and veliparib).

In certain embodiments, the anticancer or DNA damaging agent can be a biotherapeutic. Non-limiting examples of suitable biotherapeutics include rlnterferon-a₂a, rlnterferon-oi2b, rInterleukin-2, rG-CSF, rGM-CSF, and rErythropoietin.

In certain embodiments, the anticancer agent can be an antibody, such as a monoclonal antibody. Non-limiting examples of suitable therapeutic monoclonal antibodies for use in the methods described herein include trastuzumab, an anti-ErbB2/HER2 for breast cancer, cetuximab, an anti-ErbBl/EGFR for colorectal cancer, and bevacizumab, an anti-VEGF for colorectal, breast and lung cancers (G. Adams et al., Nature Biotechnology 23: 1 147-57 (2005)). Multitarget inhibitors, such as Sutent which inhibits TK activity of VEGFR, PDGFR and FGFR, are also suitable for use in the inventive method.

In certain embodiments, the anticancer agent can be a proteasome inhibitor, such as bortezomib.

Administration of the compounds disclosed herein can be accomplished via any mode of administration of therapeutic agents including systemic or local administration such as oral, nasal, parenteral, transdermal, subcutaneous, vaginal, buccal, rectal or topical administration modes.

Some patients may experience allergic reactions to the compounds disclosed herein and/or other anti-cancer agent(s) during or after administration; therefore, anti-allergic agents are often administered to minimize the risk of an allergic reaction. In certain embodiments, provided is a method of treating a disease or disorder associated with USP1 (e.g., cancer) comprising administering or coadministering, in any order, to a patient in need thereof a compound disclosed herein (e.g., a compound of Formula (I), (II) or a compound of Table 1 or pharmaceutically acceptable salts thereof) and an anti-allergic agent (e.g., corticosteroids, including, but not limited to, dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®)).

Some patients may experience nausea during and after administration of the compound disclosed herein and/or other anti-cancer agent(s); therefore, anti-emetics are used in preventing nausea (upper stomach) and vomiting. In certain embodiments, provided is a method of treating a disease or disorder associated with USP1 (e.g., cancer) comprising administering or coadministering, in any order, to a patient in need thereof a compound disclosed herein (e.g., a compound of Formula (I), (II) or a compound of Table 1 or pharmaceutically acceptable salts thereof) and an anti-emetic (e.g., aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof).

Medication to alleviate the pain experienced during the treatment period is often prescribed to make the patient more comfortable. In certain embodiments, provided is a method of treating a disease or disorder associated with USP1 (e.g., cancer) comprising administering or coadministering, in any order, to a patient in need thereof a compound disclosed herein (e.g., a compound of Formula (I), (II) or a compound of Table 1 or pharmaceutically acceptable salts thereof) and an analgesic (e.g., an over-the-counter analgesics, (e.g., Tylenol®), an opioid analgesic (e.g., hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®)).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. In certain embodiments, provided is a method of treating a disease or disorder associated with USP1 (e.g., cancer) comprising administering or coadministering, in any order, to a patient in need thereof a compound disclosed herein (e.g., a compound of Formula (I), (II) or a compound of Table 1 or pharmaceutically acceptable salts thereof) and a cytoprotective agent (e.g., Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid)).

The structure of the active compounds identified by code numbers, generic or trade names may be taken from the actual edition of the standard compendium “The Merck Index” or from databases, e.g., Patents International (e.g., IMS World Publications).

The above-mentioned compounds, which can be used in combination with a compound disclosed herein, can be prepared and administered as described in the art, including, but not limited to, in the documents cited above.

In some embodiments, provided are pharmaceutical compositions comprising at least one compound disclosed herein (e.g., a USP1 inhibitor, e.g., a compound of Formula (I), (II) or a compound of Table 1 or a pharmaceutically acceptable salt, hydrate, solvate, prodrug, stereoisomer or tautomer thereof together with a pharmaceutically acceptable carrier suitable for administration to a human or animal subject, either alone or together with other anti-cancer agents.

In some embodiments, provided are methods of treating human or animal subjects having or having been diagnosed with a disease or disorder associated with USP1 (e.g., cancer) comprising administering to the subject in need thereof a therapeutically effective amount of a compound disclosed herein (e.g., a compound of Formula (I), (II) or a compound of Table 1 or a pharmaceutically acceptable salt, hydrate, solvate, prodrug, stereoisomer or tautomer thereof in combination with a second therapeutic agent.

In some embodiments, provided are methods of treating a a disease or disorder associated with USP1 (e.g., cancer) in a subject in need thereof comprising administering to the subject a therapeutically effective amount of a compound disclosed herein (e.g., a compound of Formula (I), (II) or a compound of Table 1 or a pharmaceutically acceptable salt, hydrate, solvate, prodrug, stereoisomer or tautomer thereof in combination with a second therapeutic agent.

In particular, compositions will either be formulated together as a combination therapeutic or administered separately.

In combination therapy, the compound disclosed herein and other anti-cancer agent(s) may be administered either simultaneously, concurrently or sequentially with no specific time limits, wherein such administration provides therapeutically effective levels of the two compounds in the body of the patient.

A compound disclosed herein (e.g., a compound of Formula (I), (II) or a compound of Table 1 or a pharmaceutically acceptable salt, hydrate, solvate, prodrug, stereoisomer or tautomer thereof) may also be used in combination with known therapeutic processes, for example, the administration of hormones or especially radiation. A compound disclosed herein may in particular be used as a radiosensitizer, especially for the treatment of tumors which exhibit poor sensitivity to radiotherapy.

In certain instances, compounds disclosed herein are combined with other therapeutic agents, including, but not limited to, other anti-cancer agents, anti-allergic agents, anti-nausea agents (or anti-emetics), pain relievers, cytoprotective agents, and combinations thereof.

Patient Selection and Monitoring

Determining Whether a Subject Will Respond to Treatment with USP1 Inhibitors

In some embodiments, provided is a method of determining if a subject having or having been diagnosed with a cancer (e.g., a cancer associated with USP1) (i.e., a cancer patient (e.g., a USP1-associated cancer patient)) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting levels of RAD18 and/or UBE2K (e.g., RAD18 and/or UBE2K protein and/or RAD18 and/or UBE2K mRNA) in a test cancer sample (e.g., in a cancer test sample obtained from the subject);
b) comparing the test cancer sample with reference cells (e.g., a reference sample taken from a non-cancerous or normal control subject), wherein elevated levels of RAD18 and/or UBE2K in said test cancer sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, provided is a method of determining if a subject having or having been diagnosed with a cancer (e.g., a cancer associated with USP1) (i.e., a cancer patient (e.g., a USP1-associated cancer patient)) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting levels of translesion synthesis (e.g., detecting PCNA monoubiquitination levels) in a test cancer sample (e.g., in a cancer test sample obtained from the subject);
b) comparing the test cancer sample with reference cells (e.g., a reference sample taken from a non-cancerous or normal control subject), wherein elevated translesion synthesis (e.g., increased PCNA monoubiquitination levels) in said test cancer sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, provided is a method of determining if a subject having or having been diagnosed with a cancer (e.g., a cancer associated with USP1) (i.e., a cancer patient (e.g., a USP1-associated cancer patient)) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting mutations in a gene encoding ATM (i.e., loss function mutations) in a test cancer sample (e.g., in a cancer test sample obtained from the subject);
b) wherein presence of mutations in a gene encoding ATM in said test cancer sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, provided is a method of determining if a subject having or having been diagnosed with a cancer (e.g., a cancer associated with USP1) (i.e., a cancer patient (e.g., a USP1-associated cancer patient)) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting germline or somatic mutations in a gene encoding BRCA1 (e.g., a loss of function mutation) in a subject test sample (e.g., in a cancer test sample or blood test sample obtained from the subject);
b) wherein presence of mutations in a gene encoding BRCA1 in said test sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, provided is a method of determining if a subject having or having been diagnosed with a cancer (e.g., a cancer patient) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting germline or somatic mutations in a gene encoding BRCA2 (e.g., a loss of function mutation) in a subject test sample (e.g., in a cancer test sample or blood test sample obtained from the subject);
b) wherein presence of mutations in a gene encoding BRCA2 in said test sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, provided is a method of determining if a subject having or having been diagnosed with a cancer (e.g., a cancer associated with USP1) (i.e., a cancer patient (e.g., a USP1-associated cancer patient)) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting deficiency in homologous recombination (e.g., as measured by a positive homologous recombination deficiency (HRD) score) in a subject test sample (e.g., in a cancer sample or blood sample obtained from the subject);
b) wherein presence of homologous recombination deficiency (e.g., a positive homologous recombination deficiency (HRD) score) in said test sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, the cancer is a cancer selected from the cancers disclosed herein. In some embodiments, the cancer is pancreatic cancer, breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)). In certain embodiments, the cancer is breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)).

Determining if a Cancer Will Respond to Treatment with a USP1 Inhibitor

In some embodiments, provided is a method of determining if a cancer (e.g., a cancer associated with USP1) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting levels of RAD18 and/or UBE2K (e.g., RAD18 and/or UBE2K protein and/or RAD18 and/or UBE2K mRNA) a cancer test sample (e.g., in a cancer sample obtained from the subject);
b) comparing the cancer test sample with a reference (e.g., a reference sample taken from a non-cancerous or normal control subject), wherein elevated levels of RAD18 and/or UBE2K in said test sample indicates that the cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, provided is a method of determining if a cancer (e.g., a cancer associated with USP1) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting levels of translesion synthesis (e.g., detecting PCNA monoubiquitination levels) in a test cancer sample (e.g., in a cancer sample obtained from the subject);
b) comparing the test cancer sample with a reference (e.g., a reference sample taken from a non-cancerous or normal control subject), wherein elevated translesion synthesis (e.g., increased PCNA monoubiquitination levels) in said test cancer sample indicates that the cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, provided is a method of determining if a cancer (e.g., a cancer associated with USP1) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting mutations in a gene encoding ATM (i.e., loss function mutations) in a test cancer sample (e.g., in a cancer sample obtained from the subject);
b) wherein presence of mutations in a gene encoding ATM in said cancer sample indicates that the cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, provided is a method of determining if a cancer (e.g., a cancer associated with USP1) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting germline or somatic mutations in a gene encoding BRCA1 (e.g., a loss of function mutation) in a cancer subject test sample (e.g., in a cancer sample or blood sample obtained from the cancer subject);
b) wherein presence of mutations in a gene encoding BRCA1 in said test sample indicates that the subject's cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, provided is a method of determining if a cancer (e.g., a cancer associated with USP1) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting germline or somatic mutations in a gene encoding BRCA2 (e.g., a loss of function mutation) in a cancer subject test sample (e.g., in a cancer sample or blood sample obtained from the cancer subject);
b) wherein presence of mutations in a gene encoding BRCA2 in said test sample indicates that the subject's cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, provided is a method of determining if a cancer (e.g., a cancer associated with USP1) will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof), comprising the steps of:
a) detecting deficiency in homologous recombination (e.g., as measured by a positive homologous recombination deficiency (HRD) score) in a cancer subject test sample (e.g., in a cancer sample or blood sample obtained from the cancer subject);
b) wherein presence of homologous recombination deficiency in said test sample indicates that the subject's cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof).

In some embodiments, the cancer is a cancer selected from the cancers disclosed herein. In some embodiments, the cancer is pancreatic cancer, breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)). In certain embodiments, the cancer is breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)).

Determining Sensitivity of a Cancer Cell to USP1 Inhibition

In some embodiments, provided is a method of determining the sensitivity of a cancer cell to USP1 inhibition (e.g., inhibition with a compound of Formula (I), (II) or a compound of Table 1 or a pharmaceutically acceptable salt, hydrate, solvate, prodrug, stereoisomer or tautomer thereof), comprising the steps of:
a) detecting levels of RAD18 and/or UBE2K (e.g., RAD18 and/or UBE2K protein and/or RAD18 and/or UBE2K mRNA) in a cancer cell test sample (e.g., in a cancer sample obtained from the subject);
b) comparing the test sample with a reference (e.g., a reference sample taken from a non-cancerous or normal control subject), wherein elevated levels of RAD18 and/or UBE2K in said test sample indicates said cancer cell is sensitive to USP1 inhibition.

In some embodiments, provided is a method of determining the sensitivity of a cancer cell to USP1 inhibition (e.g., inhibition with a compound of Formula (I), (II) or a compound of Table 1 or a pharmaceutically acceptable salt, hydrate, solvate, prodrug, stereoisomer or tautomer thereof), comprising the steps of:
a) detecting levels of translesion synthesis (e.g., detecting PCNA monoubiquitination levels) in a cancer cell test sample (e.g., in a cancer sample obtained from the subject);
b) comparing the test sample with a reference (e.g., a reference sample taken from a non-cancerous or normal control subject), wherein elevated translesion synthesis (e.g., increased PCNA monoubiquitination levels) in said test sample indicates said cancer cell is sensitive to USP1 inhibition.

In some embodiments, provided is a method of determining the sensitivity of a cancer cell to USP1 inhibition (e.g., inhibition with a compound of Formula (I), (II) or a compound of Table 1 or a pharmaceutically acceptable salt, hydrate, solvate, prodrug, stereoisomer or tautomer thereof), comprising the steps of:
a) detecting mutations in a gene encoding ATM (i.e., loss function mutations) in a cancer cell test sample (e.g., in a cancer sample obtained from a subject);
b) wherein presence of mutations in a gene encoding ATM in said cancer cell test sample indicates said cancer cell is sensitive to USP1 inhibition.

In some embodiments, provided is a method of determining the sensitivity of a cancer cell to USP1 inhibition (e.g., inhibition with a compound of Formula (I), (II) or a compound of Table 1 or a pharmaceutically acceptable salt, hydrate, solvate, prodrug, stereoisomer or tautomer thereof), comprising the steps of:
a) detecting a mutation in a gene encoding BRCA1 (e.g., a loss of function mutation) in a cancer cell test sample (e.g., in a cancer sample obtained from a subject);
b) wherein presence of mutations in a gene encoding BRCA1 in said cancer cell test sample indicates said cancer cell is sensitive to USP1 inhibition.

In some embodiments, provided is a method of determining the sensitivity of a cancer cell to USP1 inhibition (e.g., inhibition with a compound of Formula (I), (II) or a compound of Table 1 or a pharmaceutically acceptable salt, hydrate, solvate, prodrug, stereoisomer or tautomer thereof), comprising the steps of:
a) detecting a mutation in a gene encoding BRCA2 (e.g., a loss of function mutation) in a cancer cell test sample (e.g., in a cancer sample obtained from a subject);
b) wherein presence of mutations in a gene encoding BRCA2 in said cancer cell test sample indicates said cancer cell is sensitive to USP1 inhibition.

In some embodiments, provided is a method of determining the sensitivity of a cancer cell to USP1 inhibition (e.g., inhibition with a compound of Formula (I), (II) or a compound of Table 1 or a pharmaceutically acceptable salt, hydrate, solvate, prodrug, stereoisomer or tautomer thereof), comprising the steps of:
a) detecting deficiency in homologous recombination (e.g., as measured by a positive homologous recombination deficiency (HRD) score) in a cancer cell test sample (e.g., in a cancer sample obtained from a subject);
b) wherein presence of homologous recombination deficiency in said cancer cell test sample indicates said cancer cell is sensitive to USP1 inhibition.

In some embodiments, the cancer is a cancer selected from the cancers disclosed herein. In some embodiments, the cancer is pancreatic cancer, breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)). In certain embodiments, the cancer is breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)).

Therapeutic Methods for Treating Subjects Having or Having been Diagnosed with Cancer

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of:
a) detecting levels of RAD18 and/or UBE2K (e.g., RAD18 and/or UBE2K protein and/or RAD18 and/or UBE2K mRNA) in a test cancer sample (e.g., in a cancer test sample obtained from the subject);
b) comparing the test cancer sample with reference cells (e.g., a reference sample taken from a non-cancerous or normal control subject), wherein elevated levels of RAD18 and/or UBE2K in said test cancer sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject identified in step b).

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of:
a) detecting levels of translesion synthesis (e.g., detecting PCNA monoubiquitination levels) in a test cancer sample (e.g., in a cancer test sample obtained from the subject);
b) comparing the test cancer sample with reference cells (e.g., a reference sample taken from a non-cancerous or normal control subject), wherein elevated translesion synthesis (e.g., increased PCNA monoubiquitination levels) in said test cancer sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject identified in step b).

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of:
a) detecting mutations in a gene encoding ATM (i.e., loss function mutations) in a test cancer sample (e.g., in a cancer test sample obtained from the subject);
b) wherein presence of mutations in a gene encoding ATM in said test cancer sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject identified in step b).

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of:
a) detecting germline or somatic mutations in a gene encoding BRCA1 (e.g., a loss of function mutation) in a subject test sample (e.g., in a cancer test sample or blood test sample obtained from the subject);
b) wherein presence of mutations in a gene encoding BRCA1 in said test sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject identified in step b).

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of:
a) detecting germline or somatic mutations in a gene encoding BRCA2 (e.g., a loss of function mutation) in a subject test sample (e.g., in a cancer test sample or blood test sample obtained from the subject);
b) wherein presence of mutations in a gene encoding BRCA2 in said test sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject identified in step b).

In some embodiments provided is a therapeutic method of treating a subject having or having been diagnosed with a cancer (e.g., a cancer associated with USP1) (i.e., a cancer patient (e.g., a USP1-associated cancer patient)) comprising the steps of:
a) detecting deficiency in homologous recombination (e.g., as measured by a positive homologous recombination deficiency (HRD) score) in a subject test sample (e.g., in a cancer sample or blood sample obtained from the subject);
b) wherein presence of homologous recombination deficiency in said test sample indicates that the subject will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject identified in step b).

In some embodiments, the cancer is a cancer selected from the cancers disclosed herein. In some embodiments, the cancer is pancreatic cancer, breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)). In certain embodiments, the cancer is breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)).

Therapeutic Methods for Treating Cancer

In some embodiments provided is a therapeutic method of treating a cancer (e.g., a cancer associated with USP1) in a subject in need thereof comprising the steps of:
a) detecting levels of RAD18 and/or UBE2K (e.g., RAD18 and/or UBE2K protein and/or RAD18 and/or UBE2K mRNA) a cancer test sample (e.g., in a cancer sample obtained from the subject);
b) comparing the cancer test sample with a reference (e.g., a reference sample taken from a non-cancerous or normal control subject), wherein elevated levels of RAD18 and/or UBE2K in said test sample indicates that the subject's cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject whose cancer was identified in step b).

In some embodiments provided is a therapeutic method of treating a cancer (e.g., a cancer associated with USP1) in a subject in need thereof comprising the steps of:
a) detecting levels of translesion synthesis (e.g., detecting PCNA monoubiquitination levels) in a test cancer sample (e.g., in a cancer sample obtained from the subject);
b) comparing the test cancer sample with a reference (e.g., a reference sample taken from a non-cancerous or normal control subject), wherein elevated translesion synthesis (e.g., increased PCNA monoubiquitination levels) in said test cancer sample indicates that the subject's cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject whose cancer was identified in step b).

In some embodiments provided is a therapeutic method of treating a cancer (e.g., a cancer associated with USP1) in a subject in need thereof comprising the steps of:
a) detecting mutations in a gene encoding ATM (i.e., loss function mutations) in a test cancer sample (e.g., in a cancer sample obtained from the subject);
b) wherein presence of mutations in a gene encoding ATM in said cancer sample indicates that the subject's cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject whose cancer was identified in step b).

In some embodiments provided is a therapeutic method of treating a cancer (e.g., a cancer associated with USP1) in a subject in need thereof comprising the steps of:
a) detecting germline or somatic mutations in a gene encoding BRCA1 (e.g., a loss of function mutation) in a cancer subject test sample (e.g., in a cancer sample or blood sample obtained from the cancer subject);
b) wherein presence of mutations in a gene encoding BRCA1 in said test sample indicates that the subject's cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject whose cancer was identified in step b).

In some embodiments provided is a therapeutic method of treating a cancer (e.g., a cancer associated with USP1) in a subject in need thereof comprising the steps of:
a) detecting germline or somatic mutations in a gene encoding BRCA2 (e.g., a loss of function mutation) in a cancer subject test sample (e.g., in a cancer sample or blood sample obtained from the cancer subject);
b) wherein presence of mutations in a gene encoding BRCA2 in said test sample indicates that the subject's cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject whose cancer was identified in step b).

In some embodiments provided is a therapeutic method of treating a cancer (e.g., a cancer associated with USP1) in a subject in need thereof comprising the steps of:
a) detecting deficiency in homologous recombination (e.g., as measured by a positive homologous recombination deficiency (HRD) score) in a cancer subject test sample (e.g., in a cancer sample or blood sample obtained from the cancer subject);
b) wherein presence of homologous recombination deficiency in said test sample indicates that the subject's cancer will respond to therapeutic treatment with a USP1 inhibitor (e.g., a compound of Formula (I), (II) or a compound of Table 1 or a pharmaceutically acceptable salt, hydrate, solvate, prodrug, stereoisomer or tautomer thereof); and
c) administering a therapeutically effective amount of USP1 inhibitor (e.g., a compound of Formula (I), (II) or a compound of Table 1 or a pharmaceutically acceptable salt, hydrate, solvate, prodrug, stereoisomer or tautomer thereof) to the subject whose cancer was identified in step b).

In some embodiments, the cancer is a cancer selected from the cancers disclosed herein. In some embodiments, the cancer is pancreatic cancer, breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)). In certain embodiments, the cancer is breast cancer (e.g., triple negative breast cancer (TNBC)), ovarian cancer (e.g., platinum-resistant ovarian cancer, platinum-refractory ovarian cancer), prostate cancer, lung cancer (e.g., non-small cell lung cancer (NSCLC)).

Sample Preparation

The disclosure further provides assays for the detection of levels of translesion synthesis (e.g., PCNA monoubiquitination levels, levels of RAD18, (e.g., RAD18 protein and/or RAD18 mRNA), UBE2K (e.g., UBE2K protein and/or UBE2K mRNA)). The disclosure further provides assays for detecting ATM mutations (e.g., ATM loss of function mutations), loss of ATM protein expression (e.g., as measured by immunohistochemistry), BRCA1 mutations (e.g., BRCA1 loss of function mutations), BRCA2 mutations (e.g., BRCA2 loss of function mutations), BRCA1/2 deficiency and deficiencies in homologous recombination (e.g., as measured by a positive homologous recombination deficiency (HRD) score). They detection of any of the above parameters can be performed in a patient sample, e.g., in a body fluid such as blood (e.g., serum or plasma) bone marrow, cerebral spinal fluid, peritoneal/pleural fluid, lymph fluid, ascite, serous fluid, sputum, lacrimal fluid, stool, and urine, or in a tissue such as a tumor tissue. The tumor tissue can be fresh tissue or preserved tissue (e.g., formalin fixed tissue, e.g., paraffin-embedded tissue).

Body fluid samples can be obtained from a subject using any of the methods known in the art. Methods for extracting cellular DNA from body fluid samples are well known in the art. Typically, cells are lysed with detergents. After cell lysis, proteins are removed from DNA using various proteases. DNA is then extracted with phenol, precipitated in alcohol, and dissolved in an aqueous solution. Methods for extracting acellular DNA from body fluid samples are also known in the art. Commonly, a cellular DNA in a body fluid sample is separated from cells, precipitated in alcohol, and dissolved in an aqueous solution.

Measurement of Gene Expression

In some embodiments, elevated levels of RAD18 and/or UBE2K are elevated RAD18 and/or UBE2K gene expression levels. In some embodiments, elevated levels of RAD18 and/or UBE2K are elevated RAD18 and/or UBE2K mRNA levels. Measurement of gene expression can be performed using any method or reagent known in the art.

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips.

In some embodiments, gene expression is detected and quantitated by hybridization to a probe that specifically hybridizes to the appropriate probe for that biomarker. The probes also can be attached to a solid support for use in high throughput screening assays using methods known in the art.

In some embodiments, the expression level of a gene is determined through exposure of a nucleic acid sample to the probe-modified chip. Extracted nucleic acid is labeled, for example, with a fluorescent tag, preferably during an amplification step.

Hybridization of the labeled sample is performed at an appropriate stringency level. The degree of probe-nucleic acid hybridization is quantitatively measured using a detection device.

Alternatively, any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination. In some embodiments, the hybridized nucleic acids are detected by detecting one or more labels attached to the sample nucleic acids. The labels can be incorporated by any of a number of means well known to those of skill in the art. However, in some embodiments, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label into the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

In one example, the gene expression can be measured through an in-situ hybridization protocol that can detect RNA molecules on a slide containing tissue sections or cells (e.g., through RNAscope®).

Detectable labels suitable for use in the methods disclosed herein include any composition detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵, ³⁵S, ¹⁴C, or ³²P) enzymes (e.g., horseradish peroxidase, alkaline phosphatase and others commonly used in an ELISA), and calorimetric labels such as colloidal gold or colored glass or plastic (e.g., polystyrene, polypropylene, latex, etc.) beads.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

In some embodiments, the detection of elevated of RAD18 and/or UBE2K mRNA levels is by quantitative reverse transcriptase (RT)-polymerase chain reaction (PCR), RNA-Seq, or microarray.

Detection of Polypeptides

Protein levels of RAD18 and/or UBE2K can be determined by examining protein expression or the protein product. Determining the protein level involves measuring the amount of any immunospecific binding that occurs between an antibody that selectively recognizes and binds to the polypeptide of the biomarker in a sample obtained from a subject and comparing this to the amount of immunospecific binding of at least one biomarker in a control sample.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, HPLC, mass spectrometry, confocal microscopy, enzymatic assays, surface plasmon resonance and PAGE-SDS.

In some embodiments, the detection of elevated RAD18 and/or UBE2K protein levels is by Western blot. In some embodiments, the detection of elevated RAD18 and/or UBE2K protein levels is by fluorescence-activated cell sorting (FACS). In some embodiments, the detection of elevated RAD18 and/or UBE2K protein levels is by immunohistochemistry.

Other Detection Methods

Mutations in targets of interest (e.g., BRCA1 mutations, BRCA2 mutations, ATM mutations) can be detected by methods known to those of skill in the art.

For detection of germline mutation, DNA sequencing may be performed using DNA extract from body fluid such as blood (e.g., serum or plasma) bone marrow, cerebral spinal fluid, peritoneal/pleural fluid, lymph fluid, ascite, serous fluid, sputum, lacrimal fluid, stool, and urine. Alternatively, sequencing may be performed on DNA extracted from a tissue such as a tumor tissue. The tumor tissue can be fresh tissue or preserved tissue (e.g., formalin fixed tissue, e.g.paraffin-embedded tissue). Sequencing may also be performed using cell-free DNA. The coding regions and sometimes adjacent regions (e.g., introns, promoter) of genes of interest are sequenced using next generation sequencing (NGS) or Sanger sequencing (Genetic/Familial High-Risk Assessment: Breast, Ovarian, and Pancreatic, Version 2.2021, NCCN Clinical Practice Guidelines in Oncology, ESMO guideline for BRCA testing DOI: 10.1093/annonc/mdw327, Clinical testing of BRCA1 and BRCA2: a worldwide snapshot of technological practices). Loss of function mutations or gene rearrangements may be detected or validated using secondary methods such as qPCR, PCR, immunohistochemistry, Sanger sequencing, comparative genomic hybridization, or the PacBio system.

Deficiencies in homologous recombination can be identified by methods known to those of skill in the art. One indicator of homologous recombination deficiencies is genomic instability (e.g., represented by a positive homologous recombination deficiency (HRD) score), which can be quantified by methods known in the art (see, e.g., Pikor L, et al., Cancer Metastasis Rev. 2013; 32(3-4):341-352). HRD score is measured using next generation sequencing of DNA extracted from tumor tissues (fresh or FFPE), based on genomic instability (e.g., loss of heterozygosity, telomeric allelic imbalance, and large-scale state transitions). Commercial FDA-approved assays are available for such measures (Myriad and Foundation Medicine).

Kits

In some embodiments kits related to methods disclosed herein are provided.

In some embodiments, a kit for predicting the sensitivity of a subject having or having been diagnosed with a disease or disorder associated with USP1 for treatment with a USP1 inhibitor is provided. The kit comprises: i) reagents capable of detecting human cancer cells associated with a disease or disorder associated with USP1 (e.g., reagents capable of specifically detecting RAD18 and/or UBE2K) and ii) instructions for how to use said kit.

In some embodiments, the present disclosure provides kit, comprising: (a) a pharmaceutical composition comprising a USP1 inhibitor and one or more pharmaceutically acceptable excipients, and (b) a diagnostic kit comprising at least one agent capable of specifically detecting RAD18 and/or UBE2K.

In some embodiments, the agent capable of specifically detecting RAD18 and/or UBE2K is capable of specifically hybridizing to RAD18 and/or UBE2K mRNA. In some embodiments, the agent capable of specifically detecting RAD18 and/or UBE2K is capable of specifically binding to RAD18 and/or UBE2K protein.

In another embodiment, the present disclosure provides kits which comprise a compound disclosed herein (or a composition comprising a compound disclosed herein) packaged in a manner that facilitates their use to practice methods of the present disclosure. In some embodiments, the kit includes a compound disclosed herein (or a composition comprising a compound disclosed herein) packaged in a container, such as a sealed bottle or vessel, with a label affixed to the container or included in the kit that describes use of the compound or composition to practice the method of the disclosure. In some embodiments, the compound or composition is packaged in a unit dosage form. The kit further can include a device suitable for administering the composition according to the intended route of administration. In some embodiments, the present disclosure provides a kit which comprise a compound disclosed herein, or a pharmaceutically acceptable salt or solvate thereof, and instructions for administering the compound, or a pharmaceutically acceptable salt or solvate thereof, to a patient having cancer.

### SELECTED EMBODIMENTS

Embodiment 1. A compound of Formula (I) or a pharmaceutically acceptable salt, hydrate, solvate, prodrug, stereoisomer, or tautomer thereof;

wherein:

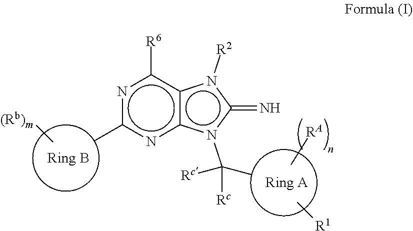

Ring B is a 5-6 member monocyclic aryl or heteroaryl;
Ring A is selected from C₆-C₁₀ aryl, 5-10 membered heteroaryl, —C₃-C₁₀ cycloalkyl, and 3-10 membered heterocyclyl;
R¹ is an optionally substituted 5-10 membered heteroaryl or an optionally substituted 3-10 membered heterocyclyl;
R² is selected from H, —C₁-C₆ alkyl, —C₁-C₆ haloalkyl, —C₁-C₆ heteroalkyl, —C₁-C₆ hydroxyalkyl, —C₃-C₁₀ cycloalkyl and arylalkyl, wherein each hydrogen of the alkyl, haloalkyl, heteroalkyl, hydroxylalkyl and arylalkyl can be independently replaced with a deuterium atom;
R⁶ is selected from H, -D, halo, —CN, —C₁-C₆ alkyl, —C₁-C₆ alkynyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, 6-10 member heteroaryl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, and —OC(═O)N(R^{a6})₂, wherein each alkyl, alkynyl, cycloalkyl, heterocyclyl, aryl, heteroaryl, arylalkyl and heteroarylalkyl is optionally substituted at any available position;
each R^{a6} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl;
each R^{A} is independently selected from -D, halo, —CN, —C₁-C₆ alkyl, —C₁-C₆ haloalkyl, —C₁-C₆ hydroxyalkyl, —C₃-C₁₀ cycloalkyl, —OR^{A1}, —N(R^{A1})₂;
each R^{A1} is independently selected from H, —C₁-C₆ alkyl, —C₁-C₆ haloalkyl and C₃-C₉ cycloalkyl;
each R^{b} is independently selected from D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{b}, —N(R^{b1})₂, —C(═O)R^{b1}, —C(═O)OR^{b1}, —NRIC(═O)R^{b1}, —NR^{b1}C(═O)OR^{b1}, —C(═O)N(R^{b1})₂, —OC(═O)N(R^{b1})₂, —S(═O)R^{b1}, —S(═O)₂R^{b1}, —SR¹, —S(═O)(═NR^{b1})R^{b1}, —NR^{b1}S(═O)₂R^{b1} and —S(═O)₂N(R^{b1})₂ or 2 R^{b} together with the atoms to which they are attached form a 4-7 member carbocyclyl or a 4-7 member heterocyclyl, wherein each alkyl, carbocylyl, cycloalkyl, heterocyclyl, aryl, heteroaryl, arylalkyl and heteroarylalkyl of R^{b} is optionally substituted at any available position;
each R^{b1} is independently selected from H, —C₁-C₆ alkyl (wherein each hydrogen can be independently replaced by deuterium), —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl;
each R^{c} and R^{c′} is independently selected from H, -D, —C₁-C₆ alkyl (e.g., -Me), —C₁-C₆ heteroalkyl and —C₁-C₆ haloalkyl or R^{c} and R^{c′} can be taken together with the atom to which they are attached to form a —C₃-C₉ cycloalkyl (e.g., cyclopropyl) or a carbonyl;
n is 0, 1, 2 or 3; and
m is 0, 1, 2 or 3.

Embodiment 2. The compound of embodiment 1 or a pharmaceutically acceptable salt, hydrate, solvate, prodrug, stereoisomer, or tautomer thereof, wherein m is 0, 1, or 2.

Embodiment 3. The compound of embodiment 1 or a pharmaceutically acceptable salt, hydrate, solvate, prodrug, stereoisomer, or tautomer thereof, wherein m is 1 or 2.

Embodiment 4. The compound of embodiment 1 or a pharmaceutically acceptable salt, hydrate, solvate, prodrug, stereoisomer, or tautomer thereof, wherein m is 1.

Embodiment 5. The compound of embodiment 1 or a pharmaceutically acceptable salt, hydrate, solvate, prodrug, stereoisomer, or tautomer thereof, wherein m is 2.

Embodiment 6. The compound of any one of embodiments 1 to 5 or a pharmaceutically acceptable salt, hydrate, solvate, prodrug, stereoisomer, or tautomer thereof, wherein each R^{b} is independently selected from —CN, halo, —C₁-C₆ alkenyl, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, —OR^{b1} and —N(R^{b1})₂, or 2 R^{b} together with the atoms to which they are attached form a 4-7 member carbocyclyl or a 4-7 member heterocyclyl, wherein each aryl, alkyl, carbocyclyl, cycloalkyl and heterocyclyl is substituted with 0, 1, 2 or 3 instances of halo (e.g., —F, —Cl), —OH, —CN, -Me, -Et, —NH₂ or oxo and wherein each R^{b1} is independently selected from H, —C₁-C₆ alkyl (wherein each hydrogen can be independently replaced by deuterium), —C₁-C₆ heteroalkyl, —C₁-C₆ haloalkyl and C₃-C₉ cycloalkyl.

Embodiment 7. The compound of any one of embodiments 1 to 5 or a pharmaceutically acceptable salt, hydrate, solvate, prodrug, stereoisomer, or tautomer thereof, wherein each R^{b} is independently selected from halo (e.g., —Cl, —F), —CN, —C₁-C₆ alkenyl (e.g., vinyl, propenyl), —C₁-C₆ alkyl (e.g., -Me, -Et, —Pr, -ⁱPr, -ⁿBu, -sec-Bu, -iso-Bu, -^{t}Bu), —C₆-C₁₀ aryl (e.g., phenyl), —C₁-C₆ heteroalkyl (e.g., —CH₂NHCH₂CH₃, —CH₂N(CH₃)CH₂CH₃, —CH₂N(CH₃)₂), —C₁-C₆ haloalkyl (e.g., —CF₃, —CHF₂, —CH₂CF₃), —C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrrolidinyl, piperidinyl, piperazinyl, morpholinyl, 6-oxa-1-azaspiro[3.3]heptanyl, 6-oxa-1-azaspiro[3.4]octanyl), —OR^{b1} and —N(R^{b1})₂, or 2 R^{b} together with the atoms to which they are attached form a 4-7 member carbocyclyl or a 4-7 member heterocyclyl, wherein each aryl, alkyl, carbocyclyl, cycloalkyl and heterocyclyl is substituted with 0, 1, 2 or 3 instances of halo (e.g., —F, —Cl) or -Me, and wherein each R^{b1} is independently selected from H, —C₁-C₆ alkyl (wherein each hydrogen can be independently replaced by deuterium) (e.g., -Me, -Et, —Pr, -ⁱPr, -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and —C₃-C₉ cycloalkyl (e.g., cyclopropyl, cyclobutyl, cyclopentyl, cyclohexyl, cycloheptyl).

Embodiment 8. The compound of any one of embodiments 1 to 5 or a pharmaceutically acceptable salt, hydrate, solvate, prodrug, stereoisomer, or tautomer thereof, wherein each R^{b} is independently selected from —CN, —C(═CH₂)CH₃, —C(CH₃)CH₂CH₃, —Cl, —F, -Me, -ⁱPr, —CH₂N(CH₃)CH₂CF₃, —CH₂N(CH₃)₂, —CH₂OH, —CH(OH)CF₃, —CF₃, —CH₂CF₃, cyclopropyl (substituted with 0, 1, or 2 instances of —F, -Me, —CN), azetidinyl (substituted with 0 or 1 instances of —F), phenyl (substituted with 0 or 1 instances of halo), —OCF₃, —OCH₂CF₃, —OCHF₂, —OⁱPr, —OMe, —OCH₂CH(CH₃)₃, —N(Me)₂ and —NHMe, or 2 R^{b} together with the atoms to which they are attached form 1,3-dioxole substituted with 0, 1 or 2 instances of —F or -Me.

Embodiment 9. The compound of any one of embodiments 1 to 5 or a pharmaceutically acceptable salt, hydrate, solvate, prodrug, stereoisomer, or tautomer thereof, wherein each R^{b} is independently selected from —CN, —C(═CH₂)CH₃, —F, -ⁱPr, —CF₃, cyclopropyl (substituted with 0, 1 or 2 instances of —F, -Me, —CN), —OCF₃, —OCHF₂, and —OMe.

Embodiment 10. The compound of any one of embodiments 1 to 9 or a pharmaceutically acceptable salt, hydrate, solvate, prodrug, stereoisomer, or tautomer thereof, wherein Ring B is a 5-membered heteroaryl containing 1-3 heteroatoms independently selected from O, N and S.

Embodiment 11. The compound of embodiment 10 or a pharmaceutically acceptable salt, hydrate, solvate, prodrug, stereoisomer, or tautomer thereof, wherein ring B is selected from pyrrolyl, furanyl, thiophenyl, imidazolyl, pyrazolyl, oxazolyl, isoxazolyl, thiazolyl, triazolyl, oxadiazolyl, thiadiazolyl.

Embodiment 12. The compound of embodiment 10 or a pharmaceutically acceptable salt, hydrate, solvate, prodrug, stereoisomer, or tautomer thereof, wherein ring B is selected from pyrazoyle, isoxazolyl and isothiazolyl.

Embodiment 13. The compound of any one of embodiments 1 to 9 or a pharmaceutically acceptable salt, hydrate, solvate, prodrug, stereoisomer, or tautomer thereof, wherein Ring B is a 6 membered heteroaryl containing 1-3 nitrogen atoms.

Embodiment 14. The compound of embodiment 13 or a pharmaceutically acceptable salt, hydrate, solvate, prodrug, stereoisomer, or tautomer thereof, wherein Ring B is selected from pyridinyl, pyrimidinyl, pyrazinyl, triazinyl and pyridazinyl.

Embodiment 15. The compound of embodiment 13 or a pharmaceutically acceptable salt, hydrate, solvate, prodrug, stereoisomer, or tautomer thereof, wherein Ring B is selected from pyridinyl and pyrimidinyl.

Embodiment 16. The compound of any one of embodiments 1 to 8 or a pharmaceutically acceptable salt, hydrate, solvate, prodrug, stereoisomer, or tautomer thereof, wherein Ring B is selected from phenyl, pyridinyl and pyrimidinyl.

Embodiment 17. The compound of any one of embodiments 1 to 8 or a pharmaceutically acceptable salt, hydrate, solvate, prodrug, stereoisomer, or tautomer thereof, wherein Ring B is phenyl.

Embodiment 18. A compound of any one of embodiments 1 to 9 or a pharmaceutically acceptable salt, hydrate, solvate, prodrug, stereoisomer, or tautomer thereof, wherein the compound is of Formula (II) wherein:

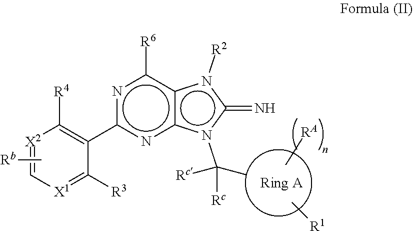

X¹ is selected from CH and N;
X² is selected from CH and N;
R³ is selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂ wherein each alkyl, cycloalkyl, heterocyclyl, aryl, heteroaryl, arylalkyl and heteroarylalkyl is optionally substituted at any available position;
R⁴ is selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂ wherein each alkyl, cycloalkyl, heterocyclyl, aryl, heteroaryl, arylalkyl and heteroarylalkyl is optionally substituted at any available position; and
each R^{a3} and R^{a4} is independently selected from H, —C₁-C₆ alkyl (wherein each hydrogen can be replaced by deuterium), —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

Embodiment 19. The compound of embodiment 18 or a pharmaceutically acceptable salt, hydrate, solvate, prodrug, stereoisomer, or tautomer thereof, wherein X¹ is CH.

Embodiment 20. The compound of embodiment 18 or a pharmaceutically acceptable salt, hydrate, solvate, prodrug, stereoisomer, or tautomer thereof, wherein X¹ is N.

Embodiment 21. The compound of any one of embodiments 18 to 20 or a pharmaceutically acceptable salt, hydrate, solvate, prodrug, stereoisomer, or tautomer thereof, wherein X² is CH.

Embodiment 22. The compound of any one of embodiments 18 to 20 or a pharmaceutically acceptable salt, hydrate, solvate, prodrug, stereoisomer, or tautomer thereof, wherein X² is N.

Embodiment 23. The compound of any one of embodiments 18 to 22 or a pharmaceutically acceptable salt, hydrate, solvate, prodrug, stereoisomer, or tautomer thereof, wherein R^{b} is selected from halo (e.g., —F), —CN, and -Me.

Embodiment 24. The compound of any one of embodiments 18 to 23 or a pharmaceutically acceptable salt, hydrate, solvate, prodrug, stereoisomer, or tautomer thereof, wherein the moiety represented by

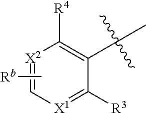

is selected from:

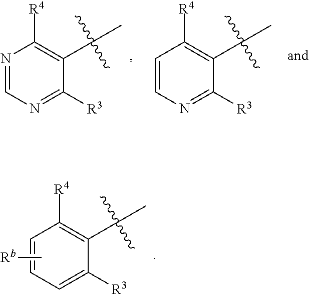

Embodiment 25. The compound of any one of embodiments 18 to 24 or a pharmaceutically acceptable salt, hydrate, solvate, prodrug, stereoisomer, or tautomer thereof, wherein each R³ is independently selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, —OR^{a3} and —N(R^{a3})₂, wherein each aryl, alkyl, cycloalkyl and heterocyclyl is substituted with 0, 1, 2 or 3 instances of halo (e.g., —F, —Cl), —OH, —CN, -Me, -Et, —NH₂ or oxo and wherein each R^{a3} is independently selected from H, —C₁-C₆ alkyl (wherein each hydrogen can be replaced by deuterium), —C₁-C₆ heteroalkyl, —C₁-C₆ haloalkyl and —C₃-C₉ cycloalkyl.

Embodiment 26. The compound of any one of embodiments 18 to 24 or a pharmaceutically acceptable salt, hydrate, solvate, prodrug, stereoisomer, or tautomer thereof, wherein each R³ is independently selected from H, -D, halo (e.g., —F, —Cl), —CN, —C₁-C₆ alkyl (e.g., -Me, -Et, —Pr, -ⁱPr, -ⁿBu, -sec-Bu, -iso-Bu, -^{t}Bu), —C₁-C₆ alkenyl (e.g., vinyl, propenyl), —C₁-C₆ heteroalkyl (e.g., —CH₂NHCH₂CH₃, —CH₂N(CH₃)CH₂CH₃, —CH₂N(CH₃)₂), —C₁-C₆ haloalkyl (e.g., —CF₃, —CHF₂, —CH₂CF₃), —C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrrolidinyl, piperidinyl, piperazinyl, morpholinyl, 6-oxa-1-azaspiro[3.3]heptanyl, 6-oxa-1-azaspiro[3.4]octanyl), —C₆-C₁₀ aryl (e.g., phenyl), —OR^{a3} and —N(R^{a3})₂, wherein each aryl, alkyl, cycloalkyl and heterocyclyl is substituted with 0, 1, 2 or 3 instances of halo (e.g., —F, —Cl), and wherein each R^{a3} is independently selected from H, —C₁-C₆ alkyl (wherein each hydrogen can be replaced by deuterium) (e.g., -Me, -CD3, -Et, —Pr, -ⁱPr, -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and —C₃-C₉ cycloalkyl (e.g., cyclopropyl, cyclobutyl, cyclopentyl, cyclohexyl, cycloheptyl).

Embodiment 27. The compound of any one of embodiments 18 to 24 or a pharmaceutically acceptable salt, hydrate, solvate, prodrug, stereoisomer, or tautomer thereof, wherein each R³ is independently selected from H, -D, —CN, —C(═CH₂)CH₃, —C(CH₃)CH₂CH₃, —Cl, —F, -Me, -ⁱPr, —CH₂N(CH₃)CH₂CF₃, —CF₃, —CH₂CF₃, cyclopropyl (substituted with 0 or 1 instance of —CN), azetidinyl (substituted with 0 or 1 instances of —F), phenyl (substituted with 0 or 1 instances of halo), —OCF₃, —OCH₂CF₃, —OCHF₂, —OCH₂F, —OⁱPr, —OMe, -OEt, -OCD₃, —OCH₂CH(CH₃)₃, —N(Me)₂, —NHMe and —NHⁱPr.

Embodiment 28. The compound of any one of embodiments 18 to 27 wherein each R⁴ is independently selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, —OR^{a4} and —N(R^{a4})₂, wherein each aryl, alkyl, cycloalkyl and heterocyclyl is substituted with 0, 1, 2 or 3 instances of halo (e.g., —F, —Cl), —OH, —CN, -Me, -Et, —NH₂ or oxo and wherein each R^{a4} is independently selected from H, —C₁-C₆ alkyl, —C₁-C₆ heteroalkyl, —C₁-C₆ haloalkyl and —C₃-C₉ cycloalkyl.

Embodiment 29. The compound of any one of embodiments 18 to 27 or a pharmaceutically acceptable salt, hydrate, solvate, prodrug, stereoisomer, or tautomer thereof, wherein each R⁴ is independently selected from H, -D, halo (e.g., —F, —Cl), —CN, —C₁-C₆ alkyl (e.g., -Me, -Et, —Pr, -ⁱPr, -ⁿBu, -sec-Bu, -iso-Bu, -^{t}Bu), —C₁-C₆ alkenyl (e.g., vinyl, propenyl), —C₁-C₆ heteroalkyl (e.g., —CH₂NHCH₂CH₃, —CH₂N(CH₃)CH₂CH₃, —CH₂N(CH₃)₂), —C₁-C₆ haloalkyl (e.g., —CF₃, —CHF₂, —CH₂CF₃), —C₁-C₆ hydroxyalkyl (e.g., —CH₂OH), —C₃-C₁₀ cycloalkyl (e.g., cyclopropyl, cyclobutyl, cyclopentyl, cyclohexyl, cycloheptyl), 3-10 membered heterocyclyl (e.g., oxetanyl, azetidinyl, tetrahydrofuranyl, tetrahydropyranyl, pyrrolidinyl, piperidinyl, piperazinyl, morpholinyl, 6-oxa-1-azaspiro[3.3]heptanyl, 6-oxa-1-azaspiro[3.4]octanyl), —C₆-C₁₀ aryl (e.g., phenyl), —OR^{a4} and —N(R^{a4})₂, wherein each aryl, alkyl, cycloalkyl and heterocyclyl is substituted with 0, 1, 2 or 3 instances of halo (e.g., —F, —Cl) or -Me, and wherein each R^{a4} is independently selected from H, —C₁-C₆ alkyl (e.g., -Me, -Et, —Pr, -ⁱPr, -ⁿBu, -sec-Bu, -iso-Bu, -^{t}Bu), —C₁-C₆ haloalkyl (e.g., —CF₃, —CHF₂, —CH₂CF₃, —CH(CH₃)CF₃) and —C₃-C₉ cycloalkyl (e.g., cyclopropyl, cyclobutyl, cyclopentyl, cyclohexyl, cycloheptyl).

Embodiment 30. The compound of any one of embodiments 18 to 27 or a pharmaceutically acceptable salt, hydrate, solvate, prodrug, stereoisomer, or tautomer thereof, wherein each R⁴ is independently selected from H, -D, —CN, —C(═CH₂)CH₃, —C(CH₃)CH₂CH₃, —Cl, —F, -Me, -ⁱPr, —CH₂N(CH₃)CH₂CF₃, —CF₃, —CH₂CF₃, cyclopropyl (substituted with 0, 1 or 2 instances of —CN, —F, or -Me), azetidinyl (substituted with 0 or 1 instances of —F), phenyl (substituted with 0 or 1 instances of halo), —OCF₃, —OCH₂CF₃, —OCHF₂, —OⁱPr, —OMe, —OCH₂CH(CH₃)₃, —N(Me)₂ and —NHMe and —NHⁱPr.

Embodiment 31. The compound of any one of embodiments 18 to 27 or a pharmaceutically acceptable salt, hydrate, solvate, prodrug, stereoisomer, or tautomer thereof, wherein each R⁴ is selected from H and —OMe.

Embodiment 32. The compound of any one of embodiments 18 to 27 or a pharmaceutically acceptable salt, hydrate, solvate, prodrug, stereoisomer, or tautomer thereof, wherein each R⁴ is —OMe.

Embodiment 33. The compound of any one of embodiments 18 to 32 or a pharmaceutically acceptable salt, hydrate, solvate, prodrug, stereoisomer, or tautomer thereof, wherein the moiety represented by

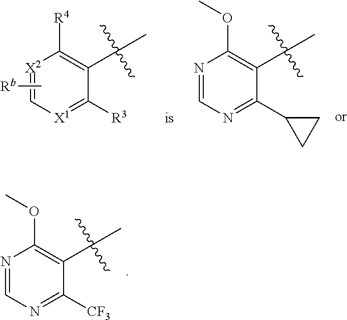

Embodiment 34. The compound of any one of embodiments 18 to 27 or a pharmaceutically acceptable salt, hydrate, solvate, prodrug, stereoisomer, or tautomer thereof, wherein each R⁴ is H.

Embodiment 35. The compound of embodiment 34 or a pharmaceutically acceptable salt, hydrate, solvate, prodrug, stereoisomer, or tautomer thereof, wherein the moiety represented by

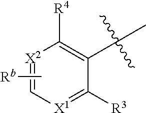

is selected from

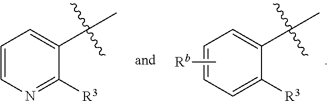

Embodiment 36. The compound of embodiment 34 or a pharmaceutically acceptable salt, hydrate, solvate, prodrug, stereoisomer, or tautomer thereof, wherein the moiety represented by

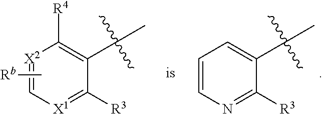

Embodiment 37. The compound of embodiment 36 or a pharmaceutically acceptable salt, hydrate, solvate, prodrug, stereoisomer, or tautomer thereof, wherein R³ is selected from cyclopropyl, —OCH₂CF₃, —OCF₃, —OCHF₂, -ⁱPr and —OMe.

Embodiment 38. The compound of embodiment 34 or a pharmaceutically acceptable salt, hydrate, solvate, prodrug, stereoisomer, or tautomer thereof, wherein the moiety represented by

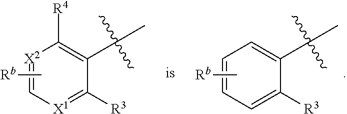

Embodiment 39. The compound of embodiment 38 or a pharmaceutically acceptable salt, hydrate, solvate, prodrug, stereoisomer, or tautomer thereof, wherein R³ is selected from —C₁, -ⁱPr, —C(═CH₂)CH₃, —OCHF₂, —OCF₃, -2-C₁-phenyl, —CF₃ and cyclopropyl.

Embodiment 40. The compound any one of embodiments 1 to 39 or a pharmaceutically acceptable salt, hydrate, solvate, prodrug, stereoisomer, or tautomer thereof, wherein R^{c} and R^{c′} are each independently selected from H and -Me or are taken together to form a cyclopropyl group.

Embodiment 41. The compound of any one of embodiments 1 to 40 or a pharmaceutically acceptable salt, hydrate, solvate, prodrug, stereoisomer, or tautomer thereof, wherein Ring A is a monocyclic 5-6 membered heteroaryl containing 1, 2 or 3 heteroatoms independently selected from N, O and S.

Embodiment 42. The compound of embodiment 41 or a pharmaceutically acceptable salt, hydrate, solvate, prodrug, stereoisomer, or tautomer thereof, wherein Ring A is a 6-membered heteroaryl containing 1-3 nitrogen atoms (e.g., pyridinyl, pyrimidinyl, pyrazinyl, triazinyl, pyridazinyl).

Embodiment 43. The compound of embodiment 41 or a pharmaceutically acceptable salt, hydrate, solvate, prodrug, stereoisomer, or tautomer thereof, wherein Ring A is pyridinyl.

Embodiment 44. The compound of embodiment 41 or a pharmaceutically acceptable salt, hydrate, solvate, prodrug, stereoisomer, or tautomer thereof, wherein Ring A is a 5-membered heteroaryl containing 1, 2 or 3 heteroatoms independently selected from N, O and S (e.g., furanyl, thiophenyl, pyrrolyl, pyrazolyl, thiazolyl, thiadiazolyl, oxazolyl, oxadiazolyl, triazolyl, thiadiazolyl, isothiazolyl, isoxazolyl, oxadiazolyl).

Embodiment 45. The compound of embodiment 44 or a pharmaceutically acceptable salt, hydrate, solvate, prodrug, stereoisomer, or tautomer thereof, wherein Ring A is thiophenyl.

Embodiment 46. The compound of any one of embodiments 1 to 40 or a pharmaceutically acceptable salt, hydrate, solvate, prodrug, stereoisomer, or tautomer thereof, wherein Ring A is a C₃-C₁₀ cycloalkyl (e.g., cyclopropyl, cyclobutyl, cyclopentyl, cyclohexyl, cycloheptyl).

Embodiment 47. The compound of any one of embodiments 1 to 40 or a pharmaceutically acceptable salt, hydrate, solvate, prodrug, stereoisomer, or tautomer thereof, wherein Ring A is cyclohexyl.

Embodiment 48. The compound of any one of embodiments 1 to 40 or a pharmaceutically acceptable salt, hydrate, solvate, prodrug, stereoisomer, or tautomer thereof, wherein Ring A is a C₆-C₁₀ aryl or a 3-10 membered heterocyclyl containing 1 or 2 heteroatoms selected from N, O and S.

Embodiment 49. The compound of any one of embodiments 1 to 40 or a pharmaceutically acceptable salt, hydrate, solvate, prodrug, stereoisomer, or tautomer thereof, wherein Ring A is phenyl.

Embodiment 50. The compound of any one of embodiments 1 to 40 or a pharmaceutically acceptable salt, hydrate, solvate, prodrug, stereoisomer, or tautomer thereof, wherein Ring A a 3-10 membered heterocyclyl containing 1 or 2 heteroatoms selected from N, O and S.

Embodiment 51. The compound of embodiment 50 or a pharmaceutically acceptable salt, hydrate, solvate, prodrug, stereoisomer, or tautomer thereof, wherein Ring A is selected from piperidinyl and piperazinyl.

Embodiment 52. The compound of any one of embodiments 1 to 40 or a pharmaceutically acceptable salt, hydrate, solvate, prodrug, stereoisomer, or tautomer thereof, wherein the moiety represented by

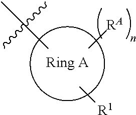

is selected from

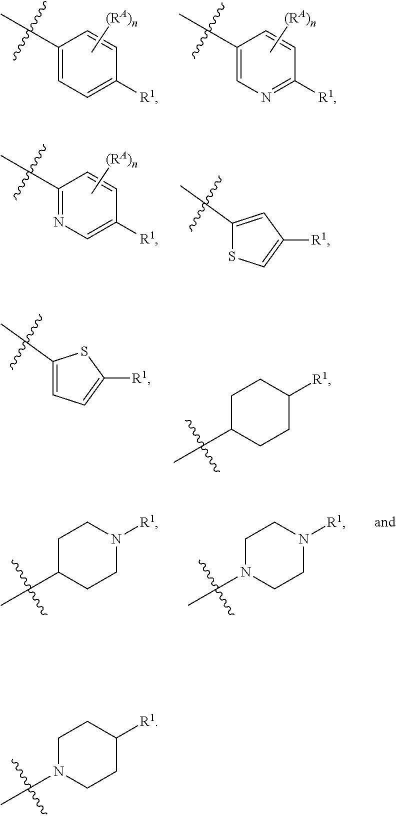

Embodiment 53. The compound of any one of embodiments 1 to 40 or a pharmaceutically acceptable salt, hydrate, solvate, prodrug, stereoisomer, or tautomer thereof, wherein the moiety represented by

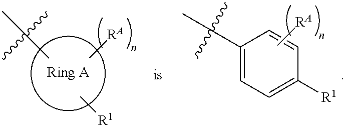

Embodiment 54. The compound of any one of embodiments 1 to 40 or a pharmaceutically acceptable salt, hydrate, solvate, prodrug, stereoisomer, or tautomer thereof, wherein the moiety represented by

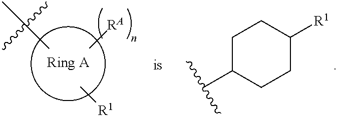

Embodiment 55. The compound of any one of embodiments 1 to 40 or a pharmaceutically acceptable salt, hydrate, solvate, prodrug, stereoisomer, or tautomer thereof, wherein the moiety represented by

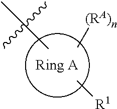

is selected from

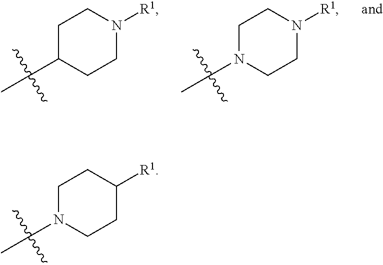

Embodiment 56. The compound of any one of embodiments 1 to 55 or a pharmaceutically acceptable salt, hydrate, solvate, prodrug, stereoisomer, or tautomer thereof, wherein n is 0.

Embodiment 57. The compound of any one of embodiments 1 to 55 or a pharmaceutically acceptable salt, hydrate, solvate, prodrug, stereoisomer, or tautomer thereof, wherein n is 1 or 2.

Embodiment 58. The compound of any one of embodiments 1 to 55 or a pharmaceutically acceptable salt, hydrate, solvate, prodrug, stereoisomer, or tautomer thereof, wherein n is 1.

Embodiment 59. The compound of any one of embodiments 1 to 55 or a pharmaceutically acceptable salt, hydrate, solvate, prodrug, stereoisomer, or tautomer thereof, wherein n is 2.

Embodiment 60. The compound of any one of embodiments 57 to 59 or a pharmaceutically acceptable salt, hydrate, solvate, prodrug, stereoisomer, or tautomer thereof, wherein each R^{A} is independently selected from -D, halo (e.g., —F, —Cl), —C₁-C₆ alkyl (e.g., -Me, -Et, —Pr, -ⁱPr, -ⁿBu, -^{t}Bu), —OH and —O—C₁-C₆ alkyl (e.g., —OMe, -OEt, —OPr, —OⁱPr, -OⁿBu, -OⁱBu).

Embodiment 61. The compound of embodiment 60 or a pharmaceutically acceptable salt, hydrate, solvate, prodrug, stereoisomer, or tautomer thereof, wherein each R^{A} is independently selected from —F, —Cl, -Me, —OH and —OMe.

Embodiment 62. The compound of any one of embodiments 1 to 56 or a pharmaceutically acceptable salt, hydrate, solvate, prodrug, stereoisomer, or tautomer thereof, wherein R¹ is a 5-10 membered heteroaryl or a 3-10 membered heterocyclyl, each substituted with 0, 1, 2 or 3 instances of R⁵, wherein each R⁵ is independently selected from halo, —CN, —C₁-C₆ alkyl, —C₁-C₆ heteroalkyl, —C₁-C₆ haloalkyl, —C₁-C₆ hydroxyalkyl, —C₃-C₁₀ cycloalkyl, 3-10 membered heterocyclyl, heterocyclylalkyl, heteroarylalkyl, arylalkyl, cycloalkylalkyl, —OR^{a5}, —N(R^{a5})₂, —C(═O)R^{a5}, —C(═O)OR^{a5}, —NR^{a5}C(═O)R^{a5}, —NR^{a5}C(═O)OR^{a5}, —C(═O)N(R^{a5})₂, —OC(═O)N(R^{a5})₂, —S(═O)R^{a5}, —S(═O)₂R^{a5}, —SR^{a5}, —S(═O)(═NR^{a5})R^{a5}, —NR^{a5}S(═O)₂R^{a5} and —S(═O)₂N(R^{a5})₂ wherein each alkyl, cycloalkyl, heterocyclyl, aryl, heteroaryl, arylalkyl and heteroarylalkyl is optionally substituted at any available position and wherein each R^{a5} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

Embodiment 63. The compound of embodiment 62 or a pharmaceutically acceptable salt, hydrate, solvate, prodrug, stereoisomer, or tautomer thereof, wherein R¹ is a 3-7 member monocyclic heterocyclyl containing 1-3 heteroatoms selected from O, N and S (e.g., azetidinyl, oxetanyl, tetrahydrofuranyl, pyrrolidinyl, piperidinyl, piperazinyl, morpholinyl, thiomorpholinyl).

Embodiment 64. The compound of embodiment 63 or a pharmaceutically acceptable salt, hydrate, solvate, prodrug, stereoisomer, or tautomer thereof, wherein R¹ is a 5-member monocyclic heterocyclyl (e.g., tetrahydrofuranyl, pyrrolidinyl).

Embodiment 65. The compound of embodiment 63 or a pharmaceutically acceptable salt, hydrate, solvate, prodrug, stereoisomer, or tautomer thereof, wherein R¹ is pyrrolidinyl.

Embodiment 66. The compound of embodiment 62 or a pharmaceutically acceptable salt, hydrate, solvate, prodrug, stereoisomer, or tautomer thereof, wherein R¹ is a 5-6 member monocyclic heteroaryl containing 1-3 heteroatoms selected from O, N and S.

Embodiment 67. The compound of embodiment 62 or a pharmaceutically acceptable salt, hydrate, solvate, prodrug, stereoisomer, or tautomer thereof, wherein R¹ is a 5 member monocyclic heteroaryl containing 1-3 heteroatoms selected from O, N and S.

Embodiment 68. The compound of embodiment 62 or a pharmaceutically acceptable salt, hydrate, solvate, prodrug, stereoisomer, or tautomer thereof, wherein R¹ is selected from pyrrolyl, pyrazolyl, imidazolyl, thiazolyl, furanyl, thiophenyl, oxazolyl, thiadiazolyl, oxadiazolyl, each substituted with 0, 1, 2 or 3 instances of R⁵.

Embodiment 69. The compound of embodiment 62 or a pharmaceutically acceptable salt, hydrate, solvate, prodrug, stereoisomer, or tautomer thereof, wherein R¹ is imidazolyl (e.g., imidazol-2-yl) or pyrazolyl (e.g., pyrazol-1-yl) substituted with 0, 1, 2 or 3 instances of R⁵.

Embodiment 70. The compound of of embodiment 62 or a pharmaceutically acceptable salt, hydrate, solvate, prodrug, stereoisomer, or tautomer thereof, wherein R¹ is pyrazolyl (e.g., pyrazol-1-yl) substituted with 0, 1, 2 or 3 instances of R⁵.

Embodiment 71. The compound of of embodiment 62 or a pharmaceutically acceptable salt, hydrate, solvate, prodrug, stereoisomer, or tautomer thereof, wherein R¹ is imidazolyl (e.g., imidazol-2-yl) substituted with 0, 1, 2 or 3 instances of R⁵.

Embodiment 72. The compound of any one of embodiments 62 to 71 or a pharmaceutically acceptable salt, hydrate, solvate, prodrug, stereoisomer, or tautomer thereof, wherein R⁵ is selected from halo (e.g., —F, —Cl, —Br), —CN, —C₁-C₆ alkyl (e.g., -Me, -Et, —Pr, -ⁱPr, -ⁿBu, -^{t}Bu), —C₁-C₆ haloalkyl (e.g., —CF₃, —CHF₂, —CH₂CF₃, —CH₂CH₂F, —CH₂CHF₂), —OC₁-C₆ alkyl (e.g., —OMe, -OEt, —OPr, —OⁱPr, -OⁿBu, —O^{t}Bu), —C₃-C₁₀ cycloalkyl (e.g., cyclopropyl, cyclobutyl, cyclopentyl, cyclohexyl, cycloheptyl) and 3-10 membered heterocyclyl (e.g., azetidinyl, oxetanyl, tetrahydrofuranyl, tetrahydropyranyl, piperazinyl, piperidinyl, morpholinyl), wherein each alkyl, cycloalky and heterocyclyl is substituted with 0, 1 or 2 instances of -Me, —OMe, —OH, —CN, halo (e.g., —F, —Cl).

Embodiment 73. The compound of any one of embodiments 62 to 71 or a pharmaceutically acceptable salt, hydrate, solvate, prodrug, stereoisomer, or tautomer thereof, wherein R⁵ is selected from —CN, —F, —Cl, —Br, -Me, -Et, -ⁱPr, —CF₃, —CH₂CH₂F, —CH₂CHF₂, —OMe, -OEt, —CH₂CH₂OMe, —CH₂CH₂OH, cyclopropyl, oxetanyl and azetidinyl (e.g., N-methyl-azetidin-3-yl).

Embodiment 74. The compound of any one of embodiments 1 to 73 or a pharmaceutically acceptable salt, hydrate, solvate, prodrug, stereoisomer, or tautomer thereof, wherein R¹ is selected from:

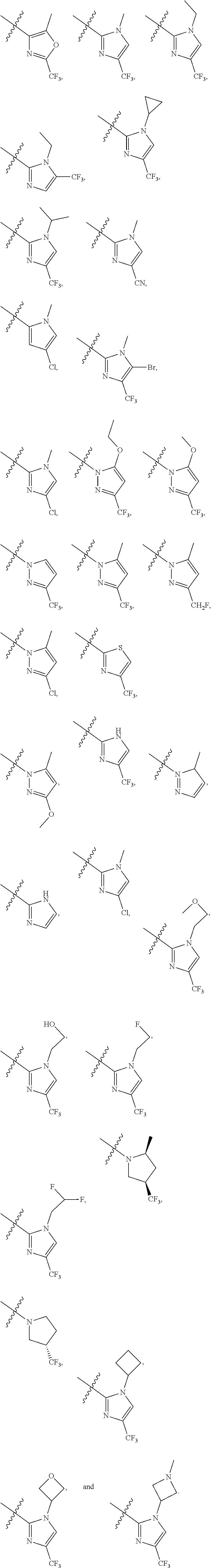

Embodiment 75. The compound of any one of embodiments 1 to 73 or a pharmaceutically acceptable salt, hydrate, solvate, prodrug, stereoisomer, or tautomer thereof, wherein R¹ is selected from:

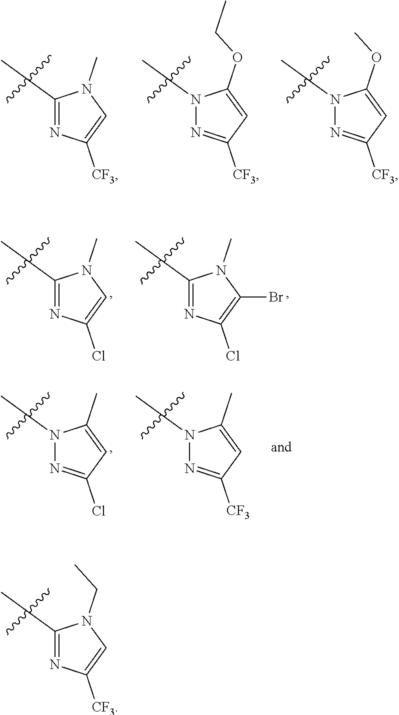

Embodiment 76. The compound of any one of embodiments 1 to 75 or a pharmaceutically acceptable salt, hydrate, solvate, prodrug, stereoisomer, or tautomer thereof, wherein R² is selected from —C₁-C₆ alkyl (e.g., -Me, -Et, —Pr, -ⁱPr, -ⁿBu, -^{t}Bu), —C₁-C₆ haloalkyl (e.g., —CF₃, —CHF₂, —CH₂CHF₂, —CH₂CF₃), —C₁-C₆ heteroalkyl (e.g., —CH₂CH₂OMe), —C₃-C₁₀ cycloalkyl (e.g., cyclopropyl, cyclobutyl, cyclopentyl, cyclohexyl) wherein each hydrogen of the alkyl, haloalkyl and heteroalkyl can be independently replaced with a deuterium atom.

Embodiment 77. The compound of any one of embodiments 1 to 75 or a pharmaceutically acceptable salt, hydrate, solvate, prodrug, stereoisomer, or tautomer thereof, wherein R² is selected from -Me, -Et, —CH₂CHF₂, —CH₂CF₃, cyclobutyl and —CH₂CH₂OMe.

Embodiment 78. The compound of any one of embodiments 1 to 75 or a pharmaceutically acceptable salt, hydrate, solvate, prodrug, stereoisomer, or tautomer thereof, wherein R² is —C₁-C₆ alkyl wherein one or more of the hydrogen atoms of the alkyl are replaced with a deuterium atom. (e.g., -CD₃, -CD₂CD₃).

Embodiment 79. The compound of embodiment 78 or a pharmaceutically acceptable salt, hydrate, solvate, prodrug, stereoisomer, or tautomer thereof, wherein R² is -CD₃.

Embodiment 80. The compound of any one of embodiments 1 to 75 or a pharmaceutically acceptable salt, hydrate, solvate, prodrug, stereoisomer, or tautomer thereof, wherein R² is -Me.

Embodiment 81. The compound of any one of embodiments 1 to 80 or a pharmaceutically acceptable salt, hydrate, solvate, prodrug, stereoisomer, or tautomer thereof, wherein R⁶ is selected from H, -D, —CN, halo (e.g., —F, —Cl), —C₁-C₆ alkyl (e.g., -Me, -Et, —Pr, -ⁱPr, -ⁿBu, -^{t}Bu), —C₁-C₆ haloalkyl (e.g., —CF₃, —CHF₂, —CH₂CF₃), —C₁-C₆ alkynyl (e.g., —CCH, —CC—CH₃, —CC-cyclopropyl), —C₆-C₁₀ aryl (e.g., phenyl substituted with 0-1 instances of C₁-C₆ alkyl), —C(═O)N(R^{a6})₂ (e.g., —C(═O)NMe2, —C(═O)NHMe, —C(═O)NH₂), —C₃-C₁₀ cycloalkyl (e.g., cyclopropyl, cyclobutyl, cyclopentyl, cyclohexyl), 6-10 member heteroaryl (e.g., pyridinyl), —N(R^{a6})₂, (e.g., —NH₂, —NMe2, —NHMe), —OH, and —O(C₁-C₆ alkyl) (e.g., —OMe).

Embodiment 82. The compound of any one of embodiments 1 to 80 or a pharmaceutically acceptable salt, hydrate, solvate, prodrug, stereoisomer, or tautomer thereof, wherein R⁶ is selected from H, -D, —CN, —F, —Cl, -Me, -Et, —Pr, -ⁱPr, -ⁿBu, -^{t}Bu, —CF₃, —CHF₂, phenyl (e.g., 2-ⁱPr-phenyl), -pyridinyl (e.g., 2-pyridinyl), —CC—CH₃, —CC-cyclopropyl, —C(═O)NMe₂, —C(═O)NHMe, —C(═O)NH₂, —NH₂, —NMe₂, —NHMe, —OH and —OMe.

Embodiment 83. The compound of any one of embodiments 1 to 80 or a pharmaceutically acceptable salt, hydrate, solvate, prodrug, stereoisomer, or tautomer thereof, wherein R⁶ is selected from H, —Cl, -Me and —CF₃.

Embodiment 84. The compound of any one of embodiments 1 to 80 or a pharmaceutically acceptable salt, hydrate, solvate, prodrug, stereoisomer, or tautomer thereof, wherein R⁶ is H.

Embodiment 85. The compound of any one of embodiments 1 to 84 or a pharmaceutically acceptable salt, hydrate, solvate, prodrug, stereoisomer, or tautomer thereof, wherein the compound is selected from the compounds of Table 1.

Embodiment 86. A pharmaceutical composition comprising a compound of any one of embodiments 1 to 88 or a pharmaceutically acceptable salt, hydrate, solvate, prodrug, stereoisomer, or tautomer thereof and a pharmaceutically acceptable carrier.

Embodiment 87. The pharmaceutical composition of embodiment 86, further comprising a second therapeutic agent.

Embodiment 88. A method for treating or preventing a disease or disorder associated with the inhibition of USP1 comprising administering to a patient in need thereof an effective amount of a compound of any one of embodiments 1 to 88 or a pharmaceutically acceptable salt, hydrate, solvate, prodrug, stereoisomer, or tautomer thereof.

Embodiment 89. A method of treating a disease or disorder associated with the inhibition of USP1 comprising administering to a patient in need thereof an effective amount (e.g., a therapeutically effective amount) of a compound of any one of embodiments 1 to 88 or a pharmaceutically acceptable salt, hydrate, solvate, prodrug, stereoisomer, or tautomer thereof.

Embodiment 90. A method for inhibiting USP1 comprising administering to a patient in need thereof an effective amount of a compound of any one of embodiments 1 to 88 or a pharmaceutically acceptable salt, hydrate, solvate, prodrug, stereoisomer, or tautomer thereof.

Embodiment 91. A method for treating or preventing cancer in a patient in need thereof comprising administering to the patient in need thereof an effective amount of a compound of any one of embodiments 1 to 88 or a pharmaceutically acceptable salt, hydrate, solvate, prodrug, stereoisomer, or tautomer thereof.

Embodiment 92. A method for treating cancer in a patient in need thereof comprising administering to the patient in need thereof an effective amount (e.g., a therapeutically effective amount) of a compound of any one of embodiments 1 to 88 or a pharmaceutically acceptable salt, hydrate, solvate, prodrug, stereoisomer, or tautomer thereof.

Embodiment 93. The method of embodiment 91 or 92, wherein the cancer is a dedifferentiated ID-driven cancer.

Embodiment 94. The method of any one of embodiments 91 to 93, wherein the cancer is a cancer that is sensitive to USP1 inhibition.

Embodiment 95. The method of any one of embodiments 91 to 94, wherein the cancer is a cancer that is sensitive to USP1 inhibition due to a dysfunctional DNA-repair pathway.

Embodiment 96. The method of any one of embodiments 91 to 95, wherein the cancer is a HRR (homologous recombination repair) gene mutant cancer.

Embodiment 97. The method of any one of embodiments 91 to 96, wherein the cancer is a HRR (homologous recombination repair) gene mutant cancer selected from the group consisting of ATM, BARD1, BRCA1, BRCA2, BRIP1, CDK12, CHEK1, CHEK2, FANCL, PALB2, PPP2R2A, RAD51B, RAD51C, RAD51D, or RAD54L mutant cancer.

Embodiment 98. The method of any one of embodiments 91 to 97, wherein the cancer is characterized by elevated levels of translesion synthesis (e.g., a cancer characterized by elevated levels of RAD18 and/or UBE2K, a cancer characterized by elevated PCNA monoubiquitination).

Embodiment 99. The method of any one of embodiments 91 to 98, wherein the cancer is characterized by a deficiency in homologous recombination (e.g., a positive homologous recombination deficiency (HRD) score).

Embodiment 100. The method of any one of embodiments 91 to 99, wherein the cancer is a BRCA1 and/or a BRCA2 mutant cancer.

Embodiment 101. The method of any one of embodiments 91 to 100, wherein the cancer is a BRCA1 and/or a BRCA2 deficient cancer.

Embodiment 102. The method of any one of embodiments 91 to 101, wherein the cancer is an ATM mutant cancer.

Embodiment 103. The method of any one of embodiments 91 to 102, wherein the cancer is an BARD1 mutant cancer.

Embodiment 104. The method of any one of embodiments 91 to 103, wherein the cancer is an BRIP1 mutant cancer.

Embodiment 105. The method of any one of embodiments 91 to 104, wherein the cancer is an CDK12 mutant cancer.

Embodiment 106. The method of any one of embodiments 91 to 105, wherein the cancer is an CHEK1 mutant cancer.

Embodiment 107. The method of any one of embodiments 91 to 106, wherein the cancer is an CHEK2 mutant cancer.

Embodiment 108. The method of any one of embodiments 91 to 107, wherein the cancer is an FANCL mutant cancer.

Embodiment 109. The method of any one of embodiments 91 to 108, wherein the cancer is an PALB2 mutant cancer.

Embodiment 110. The method of any one of embodiments 91 to 109, wherein the cancer is an PPP2R2A mutant cancer.

Embodiment 111. The method of any one of embodiments 91 to 110, wherein the cancer is an RAD51B mutant cancer.

Embodiment 112. The method of any one of embodiments 91 to 111, wherein the cancer is an RAD51C mutant cancer.

Embodiment 113. The method of any one of embodiments 91 to 112, wherein the cancer is an RAD51D mutant cancer.

Embodiment 114. The method of any one of embodiments 91 to 113, wherein the cancer is an RAD54L mutant cancer.

Embodiment 115. The method of any one of embodiments 91 to 114, wherein the cancer is a PARP inhibitor resistant or refractory cancer.

Embodiment 116. The method of any one of embodiments 91 to 115, wherein the cancer is selected from adrenocortical carcinoma, AIDS-related lymphoma, AIDS-related malignancies, anal cancer, cerebellar astrocytoma, extrahepatic bile duct cancer, bladder cancer osteosarcoma/malignant fibrous histiocytoma, brain stem glioma, ependymoma, visual pathway and hypothalamic gliomas, breast cancer, bronchial adenomas/carcinoids, carcinoid tumors, gastrointestinal carcinoid tumors, carcinoma, adrenocortical, islet cell carcinoma, primary central nervous system lymphoma, cerebellar astrocytoma, cervical cancer, chronic lymphocytic leukemia, chronic myelogenous leukemia, clear cell sarcoma of tendon sheaths, colon cancer, colorectal cancer, cutaneous t-cell lymphoma, endometrial cancer, ependymoma, esophageal cancer, Ewing's sarcoma/family of tumors, extracranial germ cell tumors, extragonadal germ cell tumors, extrahepatic bile duct cancer, eye cancers, including intraocular melanoma, and retinoblastoma, gallbladder cancer, gastrointestinal carcinoid tumor, ovarian germ cell tumor, gestational trophoblastic tumor, hairy cell leukemia, head and neck cancer, Hodgkin's disease, hypopharyngeal cancer, hypothalamic and visual pathway glioma, intraocular melanoma, Kaposi's sarcoma, laryngeal cancer, acute lymphoblastic leukemia, acute myeloid leukemia, liver cancer, non-small cell lung cancer, small cell lung cancer, non-Hodgkin's lymphoma, Waldenstrom's macroglobulinemia, malignant mesothelioma, malignant thymoma, medulloblastoma, melanoma, intraocular melanoma, merkel cell carcinoma, metastatic squamous neck cancer with occult primary, multiple endocrine neoplasia syndrome, multiple myeloma/plasma cell neoplasm, mycosis fungoides, myelodysplastic syndrome, chronic myelogenous leukemia, myeloid leukemia, multiple myeloma, myeloproliferative disorders, nasal cavity and paranasal sinus cancer, nasopharyngeal cancer, neuroblastoma, oral cancer, oral cavity and lip cancer, oropharyngeal cancer, osteosarcoma/malignant fibrous histiocytoma of bone, ovarian cancer, ovarian low malignant potential tumor, pancreatic cancer, paranasal sinus and nasal cavity cancer, parathyroid cancer, penile cancer, pheochromocytoma, pituitary tumor, pleuropulmonary blastoma, prostate cancer, rectal cancer, renal cell (kidney) cancer, transitional cell cancer (e.g., renal pelvis and ureter), retinoblastoma, rhabdomyosarcoma, salivary gland cancer, malignant fibrous histiocytoma of bone, soft tissue sarcoma, Sezary syndrome, skin cancer, small intestine cancer, stomach (gastric) cancer, supratentorial primitive neuroectodennal and pineal tumors, cutaneous t-cell lymphoma, testicular cancer, malignant thymoma, thyroid cancer, gestational trophoblastic tumor, urethral cancer, uterine sarcoma, vaginal cancer, vulvar cancer, and Wilms' tumor.

Embodiment 117. The method of any one of embodiments 91 to 116, wherein the cancer can be any cancer in any organ, for example, a cancer selected from the group consisting of glioma, thyroid carcinoma, breast carcinoma, small-cell lung carcinoma, non-small-cell carcinoma, gastric carcinoma, colon carcinoma, gastrointestinal stromal carcinoma, pancreatic carcinoma, bile duct carcinoma, CNS carcinoma, ovarian carcinoma, endometrial carcinoma, prostate carcinoma, renal carcinoma, anaplastic large-cell lymphoma, leukemia, multiple myeloma, mesothelioma, and melanoma, and combinations thereof.

Embodiment 118. The method of any one of embodiments 91 to 116, wherein the cancer is selected from liposarcoma, neuroblastoma, glioblastoma, bladder cancer, adrenocortical cancer, multiple myeloma, colorectal cancer, non-small cell lung cancer, Human Papilloma Virus-associated cervical, oropharyngeal, penis, anal, thyroid or vaginal cancer or Epstein-Barr Virus-associated nasopharyngeal carcinoma, gastric cancer, rectal cancer, thyroid cancer, breast cancer, prostate cancer, ovarian cancer, pancreatic cancer, Hodgkin lymphoma and diffuse large B-cell lymphoma.

Embodiment 119. The method of any one of embodiments 91 to 116, wherein the cancer is selected from breast cancer (e.g., triple negative breast cancer (TNBC)), ovarian cancer (e.g., platinum-resistant ovarian cancer, platinum-refractory ovarian cancer), pancreatic cancer, prostate cancer and lung cancer (e.g., non-small cell lung cancer (NSCLC)).

Embodiment 120. The method of any one of embodiments 91 to 116 wherein the cancer is selected from breast cancer (e.g., triple negative breast cancer (TNBC)), ovarian cancer (e.g., platinum-resistant ovarian cancer, platinum-refractory ovarian cancer), prostate cancer and lung cancer (e.g., non-small cell lung cancer (NSCLC)).

Embodiment 121. The method of any one of embodiments 91 to 116 wherein the cancer is breast cancer.

Embodiment 122. The method of any one of embodiments 91 to 116 wherein the cancer is triple negative breast cancer (TNBC).

Embodiment 123. The method of any one of embodiments 91 to 116 wherein the cancer is ovarian cancer.

Embodiment 124. The method of embodiment 123, wherein the cancer is platinum-resistant ovarian cancer.

Embodiment 125. The method of embodiment 123, wherein the cancer is platinum-refractory ovarian cancer.

Embodiment 126. The method of any one of embodiments 91 to 116 wherein the cancer is prostate cancer.

Embodiment 127. The method of any one of embodiments 91 to 116 wherein the cancer is lung cancer.

Embodiment 128. The method of any one of embodiments 91 to 116 wherein the cancer is non-small cell lung cancer (NSCLC).

Embodiment 129. A method for treating or preventing a disease or disorder associated with DNA damage comprising administering to a patient in need of a treatment for diseases or disorders associated with DNA damage an effective amount of a compound of any one of embodiments 1 to 88 or a pharmaceutically acceptable salt, hydrate, solvate, prodrug, stereoisomer, or tautomer thereof.

Embodiment 130. The method of embodiment 129, wherein the disease is cancer.

Embodiment 131. A method for treating a disease or disorder associated with DNA damage comprising administering to a patient in need of a treatment for diseases or disorders associated with DNA damage an effective amount (e.g., a therapeutically effective amount) of a compound of any one of embodiments 1 to 88 or a pharmaceutically acceptable salt, hydrate, solvate, prodrug, stereoisomer, or tautomer thereof.

Embodiment 132. A method of inhibiting, modulating or reducing DNA repair activity exercised by USP1 comprising administering to a patient in need thereof an effective amount of a compound of any one of embodiments 1 to 88 or a pharmaceutically acceptable salt, hydrate, solvate, prodrug, stereoisomer, or tautomer thereof.

Embodiment 133. A compound of any one of embodiments 1-88 or a pharmaceutically acceptable salt, hydrate, solvate, prodrug, stereoisomer, or tautomer thereof for use in a method for treating or preventing a disease or disorder associated with the inhibition of USP1, wherein the method comprises administering to a patient in need thereof an effective amount of the compound.

Embodiment 134. A compound of any one of embodiments 1-88 or a pharmaceutically acceptable salt, hydrate, solvate, prodrug, stereoisomer, or tautomer thereof for use in a method of treating a disease or disorder associated with the inhibition of USP1 comprising administering to a patient in need thereof an effective amount (e.g., a therapeutically effective amount) of the compound.

Embodiment 135. A compound of any one of embodiments 1-88 or a pharmaceutically acceptable salt, hydrate, solvate, prodrug, stereoisomer, or tautomer thereof for use in a method for inhibiting USP1 comprising administering to a patient in need thereof an effective amount of the compound.

Embodiment 136. A compound of any one of embodiments 1-88 or a pharmaceutically acceptable salt, hydrate, solvate, prodrug, stereoisomer, or tautomer thereof for use in a method for treating or preventing cancer in a patient in need thereof comprising administering to the patient in need thereof an effective amount of the compound.

Embodiment 137. A compound of any one of embodiments 1-88 or a pharmaceutically acceptable salt, hydrate, solvate, prodrug, stereoisomer, or tautomer thereof for use in a method for treating cancer in a patient in need thereof comprising administering to the patient in need thereof a therapeutically effective amount (e.g., a therapeutically effective amount) of the compound.

Embodiment 138. The compound for use of embodiment 136 or 137, wherein the cancer is a dediferentiated ID-driven cancer.

Embodiment 139. The compound for use of any one of embodiments 136 to 138, wherein the cancer is a cancer that is sensitive to USP1 inhibition.

Embodiment 140. The compound for use of any one of embodiments 136 to 139, wherein the cancer is a cancer that is sensitive to USP1 inhibition due to a dysfunctional DNA-repair pathway.

Embodiment 141. The compound for use of any one of embodiments 136 to 140, wherein the cancer is a HRR (homologous recombination repair) gene mutant cancer.

Embodiment 142. The compound for use of any one of embodiments 136 to 141, wherein the cancer is a HRR (homologous recombination repair) gene mutant cancer selected from the group consisting of ATM, BARD1, BRCA1, BRCA2, BRIP1, CDK12, CHEK1, CHEK2, FANCL, PALB2, PPP2R2A, RAD51B, RAD51C, RAD51D, or RAD54L mutant cancer.

Embodiment 143. The compound for use of any one of embodiments 136 to 142, wherein the cancer is characterized by elevated levels of translesion synthesis (e.g., a cancer characterized by elevated levels of RAD18 and/or UBE2K, a cancer characterized by elevated PCNA monoubiquitination).

Embodiment 144. The compound for use of any one of embodiments 136 to 143, wherein the cancer is characterized by a deficiency in homologous recombination (e.g., a positive homologous recombination deficiency (HRD) score).

Embodiment 145. The compound for use of any one of embodiments 136 to 144, wherein the cancer is a BRCA1 and/or a BRCA2 mutant cancer.

Embodiment 146. The compound for use of any one of embodiments 136 to 145, wherein the cancer is a BRCA1 and/or a BRCA2 deficient cancer.

Embodiment 147. The compound for use of any one of embodiments 136 to 146, wherein the cancer is an ATM mutant cancer.

Embodiment 148. The compound for use of any one of embodiments 136 to 147, wherein the cancer is an BARD1 mutant cancer.

Embodiment 149. The compound for use of any one of embodiments 136 to 148, wherein the cancer is an BRIP1 mutant cancer.

Embodiment 150. The compound for use of any one of embodiments 136 to 149, wherein the cancer is an CDK12 mutant cancer.

Embodiment 151. The compound for use of any one of embodiments 136 to 150, wherein the cancer is an CHEK1 mutant cancer.

Embodiment 152. The compound for use of any one of embodiments 136 to 151, wherein the cancer is an CHEK2 mutant cancer.

Embodiment 153. The compound for use of any one of embodiments 136 to 152, wherein the cancer is an FANCL mutant cancer.

Embodiment 154. The compound for use of any one of embodiments 136 to 153, wherein the cancer is an PALB2 mutant cancer.

Embodiment 155. The compound for use of any one of embodiments 136 to 154, wherein the cancer is an PPP2R2A mutant cancer.

Embodiment 156. The compound for use of any one of embodiments 136 to 155, wherein the cancer is an RAD51B mutant cancer.

Embodiment 157. The compound for use of any one of embodiments 136 to 156, wherein the cancer is an RAD51C mutant cancer.

Embodiment 158. The compound for use of any one of embodiments 136 to 157, wherein the cancer is an RAD51D mutant cancer.

Embodiment 159. The compound for use of any one of embodiments 136 to 158, wherein the cancer is an RAD54L mutant cancer.

Embodiment 160. The compound for use of any one of embodiments 136 to 159, wherein the cancer is a PARP inhibitor resistant or refractory cancer.

Embodiment 161. The compound for use of any one of embodiments 136 to 160, wherein the cancer is selected from adrenocortical carcinoma, AIDS-related lymphoma, AIDS-related malignancies, anal cancer, cerebellar astrocytoma, extrahepatic bile duct cancer, bladder cancer osteosarcoma/malignant fibrous histiocytoma, brain stem glioma, ependymoma, visual pathway and hypothalamic gliomas, breast cancer, bronchial adenomas/carcinoids, carcinoid tumors, gastrointestinal carcinoid tumors, carcinoma, adrenocortical, islet cell carcinoma, primary central nervous system lymphoma, cerebellar astrocytoma, cervical cancer, chronic lymphocytic leukemia, chronic myelogenous leukemia, clear cell sarcoma of tendon sheaths, colon cancer, colorectal cancer, cutaneous t-cell lymphoma, endometrial cancer, ependymoma, esophageal cancer, Ewing's sarcoma/family of tumors, extracranial germ cell tumors, extragonadal germ cell tumors, extrahepatic bile duct cancer, eye cancers, including intraocular melanoma, and retinoblastoma, gallbladder cancer, gastrointestinal carcinoid tumor, ovarian germ cell tumor, gestational trophoblastic tumor, hairy cell leukemia, head and neck cancer, Hodgkin's disease, hypopharyngeal cancer, hypothalamic and visual pathway glioma, intraocular melanoma, Kaposi's sarcoma, laryngeal cancer, acute lymphoblastic leukemia, acute myeloid leukemia, liver cancer, non-small cell lung cancer, small cell lung cancer, non-Hodgkin's lymphoma, Waldenstrom's macroglobulinemia, malignant mesothelioma, malignant thymoma, medulloblastoma, melanoma, intraocular melanoma, merkel cell carcinoma, metastatic squamous neck cancer with occult primary, multiple endocrine neoplasia syndrome, multiple myeloma/plasma cell neoplasm, mycosis fungoides, myelodysplastic syndrome, chronic myelogenous leukemia, myeloid leukemia, multiple myeloma, myeloproliferative disorders, nasal cavity and paranasal sinus cancer, nasopharyngeal cancer, neuroblastoma, oral cancer, oral cavity and lip cancer, oropharyngeal cancer, osteosarcoma/malignant fibrous histiocytoma of bone, ovarian cancer, ovarian low malignant potential tumor, pancreatic cancer, paranasal sinus and nasal cavity cancer, parathyroid cancer, penile cancer, pheochromocytoma, pituitary tumor, pleuropulmonary blastoma, prostate cancer, rectal cancer, renal cell (kidney) cancer, transitional cell cancer (e.g., renal pelvis and ureter), retinoblastoma, rhabdomyosarcoma, salivary gland cancer, malignant fibrous histiocytoma of bone, soft tissue sarcoma, Sezary syndrome, skin cancer, small intestine cancer, stomach (gastric) cancer, supratentorial primitive neuroectodennal and pineal tumors, cutaneous t-cell lymphoma, testicular cancer, malignant thymoma, thyroid cancer, gestational trophoblastic tumor, urethral cancer, uterine sarcoma, vaginal cancer, vulvar cancer, and Wilms' tumor.

Embodiment 162. The compound for use of any one of embodiments 136 to 160, wherein the cancer can be any cancer in any organ, for example, a cancer selected from the group consisting of glioma, thyroid carcinoma, breast carcinoma, small-cell lung carcinoma, non-small-cell carcinoma, gastric carcinoma, colon carcinoma, gastrointestinal stromal carcinoma, pancreatic carcinoma, bile duct carcinoma, CNS carcinoma, ovarian carcinoma, endometrial carcinoma, prostate carcinoma, renal carcinoma, anaplastic large-cell lymphoma, leukemia, multiple myeloma, mesothelioma, and melanoma, and combinations thereof.

Embodiment 163. The compound for use of any one of embodiments 136 to 160 wherein the cancer is selected from liposarcoma, neuroblastoma, glioblastoma, bladder cancer, adrenocortical cancer, multiple myeloma, colorectal cancer, non-small cell lung cancer, Human Papilloma Virus-associated cervical, oropharyngeal, penis, anal, thyroid or vaginal cancer or Epstein-Barr Virus-associated nasopharyngeal carcinoma, gastric cancer, rectal cancer, thyroid cancer, breast cancer, prostate cancer, ovarian cancer, pancreatic cancer, Hodgkin lymphoma and diffuse large B-cell lymphoma.

Embodiment 164. The compound for use of any one of embodiments 136 to 160 wherein the cancer is selected from breast cancer (e.g., triple negative breast cancer (TNBC)), ovarian cancer (e.g., platinum-resistant ovarian cancer, platinum-refractory ovarian cancer), pancreatic cancer, prostate cancer and lung cancer (e.g., non-small cell lung cancer (NSCLC)).

Embodiment 165. The compound for use of any one of embodiments 136 to 160 wherein the cancer is selected from breast cancer (e.g., triple negative breast cancer (TNBC)), ovarian cancer (e.g., platinum-resistant ovarian cancer, platinum-refractory ovarian cancer), prostate cancer and lung cancer (e.g., non-small cell lung cancer (NSCLC)).

Embodiment 166. The compound for use of any one of embodiments 136 to 160 wherein the cancer is breast cancer.

Embodiment 167. The compound for use of any one of embodiments 136 to 160 wherein the cancer is triple negative breast cancer (TNBC).

Embodiment 168. The compound for use of any one of embodiments 136 to 160 wherein the cancer is ovarian cancer.

Embodiment 169. The compound for use of embodiment 168, wherein the cancer is platinum-resistant ovarian cancer.

Embodiment 170. The compound for use of embodiment 168, wherein the cancer is platinum-refractory ovarian cancer.

Embodiment 171. The compound for use of any one of embodiments 136 to 160 wherein the cancer is prostate cancer.

Embodiment 172. The compound for use of any one of embodiments 136 to 160 wherein the cancer is lung cancer.

Embodiment 173. The compound for use of any one of embodiments 136 to 160 wherein the cancer is non-small cell lung cancer (NSCLC).

Embodiment 174. A compound of any one of embodiments 1-88 or a pharmaceutically acceptable salt, hydrate, solvate, prodrug, stereoisomer, or tautomer thereof for use in a method for treating or preventing a disease or disorder associated with DNA damage comprising administering to a patient in need of a treatment for diseases or disorders associated with DNA damage an effective amount of the compound.

Embodiment 175. The compound for use of embodiment 174, wherein the disease is cancer.

Embodiment 176. A compound of any one of embodiments 1-88 or a pharmaceutically acceptable salt, hydrate, solvate, prodrug, stereoisomer, or tautomer thereof for use in a method for treating a disease or disorder associated with DNA damage comprising administering to a patient in need of a treatment for diseases or disorders associated with DNA damage an effective amount (e.g., a therapeutically effective amount) of the compound.

Embodiment 177. A compound of any one of embodiments 1-88 or a pharmaceutically acceptable salt, hydrate, solvate, prodrug, stereoisomer, or tautomer thereof for use in a method of inhibiting, modulating or reducing DNA repair activity exercised by USP1 comprising administering to a patient in need thereof an effective amount of the compound.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope. In the synthetic examples below, the descriptions of experimental procedures within a reaction sequence are listed in numerical order.

### Abbreviations

### General

ADDP 1,1′-(azodicarbonyl)dipiperidine
anhy. anhydrous
aq. aqueous
satd. saturated
min(s) minute(s)
hr(s) hour(s)
mL milliliter
mmol millimole(s)
mol mole(s)
MS mass spectrometry
NMR nuclear magnetic resonance
TLC thin layer chromatography
HPLC high-performance liquid chromatography
Me methyl
i-Pr iso-propyl
Bu butyl
t-Bu tert-butyl
^{t} BuXPhos 2-di-tert-butylphosphino-2′,4′,6′-triisopropylbiphenyl
Ph phenyl
Et ethyl
Bz benzoyl
TBS t-butyldimethylsilyl
TMS trimethylsilyl
Ts p-toluenesulfonyl
RuPhos 2-dicyclohexylphosphino-2′,6′-diisopropoxybiphenyl

Spectrum
Hz hertz
δ chemical shift
J coupling constant
s singlet
d doublet
t triplet
q quartet
sept septet
m multiplet
br broad
qd quartet of doublets
dquin doublet of quintets
dd doublet of doublets
dt doublet of triplets

Solvents and Reagents
DAST Diethylaminosulfurtrifluoride
CHCl₃ chloroform
DCM dichloromethane
DMF dimethylformamide
Et₂O diethyl ether
EtOH ethyl alcohol
EtOAc ethyl acetate
MeOH methyl alcohol
MeCN acetonitrile
PE petroleum ether
THE tetrahydrofuran
DMSO dimethyl sulfoxide
t-BuOK potassium tert-butoxide
9-BBN 9-borabicyclo[3.3.1]nonane
AcOH acetic acid
FA formic acid
HCl hydrochloric acid
H₂SO₄ sulfuric acid
NH₄Cl ammonium chloride
KOH potassium hydroxide
NaOH sodium hydroxide
K₂CO₃ potassium carbonate
Na₂CO₃ sodium carbonate
Cs₂CO₃ cesium carbonate
TFA trifluoroacetic acid
Na₂SO₄ sodium sulfate
NaBH₄ sodium borohydride
NaHCO₃ sodium bicarbonate
LiHMDS lithium hexamethyldisilylamide
NaBH₄ sodium borohydride
Et₃N triethylamine
Py pyridine
PCC pyridinium chlorochromate
DMAP 4-(dimethylamino)pyridine
DIPEA N,N-diisopropylethylamine
BINAP 2,2′-bis(diphenylphosphanyl)-1,1′-binaphthyl
dppf 1,1′-bis(diphenylphosphino)ferrocene
PEP Phospho(enol)pyruvic acid
LDH Lactate Dehydrogenase
DTT DL-Dithiothreitol
BSA Bovine Serum Albumin
NADH β-Nicotinamide adenine dinucleotide, reduced
Pd(t-Bu₃P)₂ bis(tri-tert-butylphosphine)palladium(0)
AcCl acetyl chloride
i-PrMgCl Isopropylmagnesium chloride
TBSCl tert-Butyl(chloro)dimethylsilane
(i-PrO)₄Ti titanium tetraisopropoxide
BHT 2,6-di-t-butyl-4-methylphenoxide
BzCl benzoyl chloride
CsF cesium fluoride
DCC dicyclohexylcarbodiimide
DMP Dess-Martin periodinane
EtMgBr ethylmagnesium bromide
EtOAc ethyl acetate
TEA triethylamine
AlaOH alanine
TBAF tetra-n-butylammonium fluoride
TBS t-butyldimethylsilyl
TMS trimethylsilyl
TMSCF₃ (Trifluoromethyl)trimethylsilane
Bu butyl
Ti(OⁱPr)₄ tetraisopropoxytitanium
LAH Lithium Aluminium Hydride
LDA lithium diisopropylamide
LiOH·H₂O lithium hydroxide hydrates
MAD methyl aluminum bis(2,6-di-t-butyl-4-methylphenoxide)
NBS N-bromosuccinimide
Na₂SO₄ sodium sulfate
MgSO₄ magnesium sulfate
Na₂S₂O₃ sodium thiosulfate
Pet Ether petroleum ether
MeCN acetonitrile
Boc t-butoxycarbonyl
MTBE methyl tert-butyl ether
DIAD diisopropyl azodicarboxylate

### General Experimental Notes

In the following examples, the chemical reagents were purchased from commercial sources (such as Alfa, Acros, Enamine, Sigma Aldrich, TCI and Shanghai Chemical Reagent Company), and used without further purification.

Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated.

Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative pyrazoles that have been listed herein. The compounds provided herein may be prepared from known or commercially available starting materials and reagents by one skilled in the art of organic synthesis.

General Synthesis of Compounds Disclosed Herein

Compounds disclosed herein and intermediates useful for the synthesis of these compounds may be prepared by a variety of methods and techniques known to those skilled in the art. The general synthetic schemes and preparative examples shown and described below illustrate typical synthetic routes to the compounds disclosed herein and intermediates to these compounds, but as will be readily apparent to the ordinary skilled organic chemist, alternative routes may also be used for the preparation of the entire compounds or to various portions of the compounds. Starting materials and reagents used are available from commercial suppliers or can be prepared according to literature procedures using methods well known to those skilled in the art.

In the case that functional groups are present on any of the building blocks or intermediates that may interfere in reactions, these are suitably protected during the reaction in order to avoid undesired side reactions, and deprotected at the end of the synthesis. Appropriate protecting groups that can be used are extensively described in the literature, e.g., in Greene, “Protective Groups in Organic Chemistry”, John Wiley & Sons, New York (1981).

Compounds disclosed herein are prepared from commercially available starting materials using techniques and methods known in the art of synthetic organic chemistry. Intermediates and final compounds are prepared according to literature procedures and/or as illustrated in the general synthetic schemes and as detailed in the experimental part herein below.

A general route to compounds of formula (I) starting from a dichloro substituted pyrimidine is illustrated in Scheme 1.

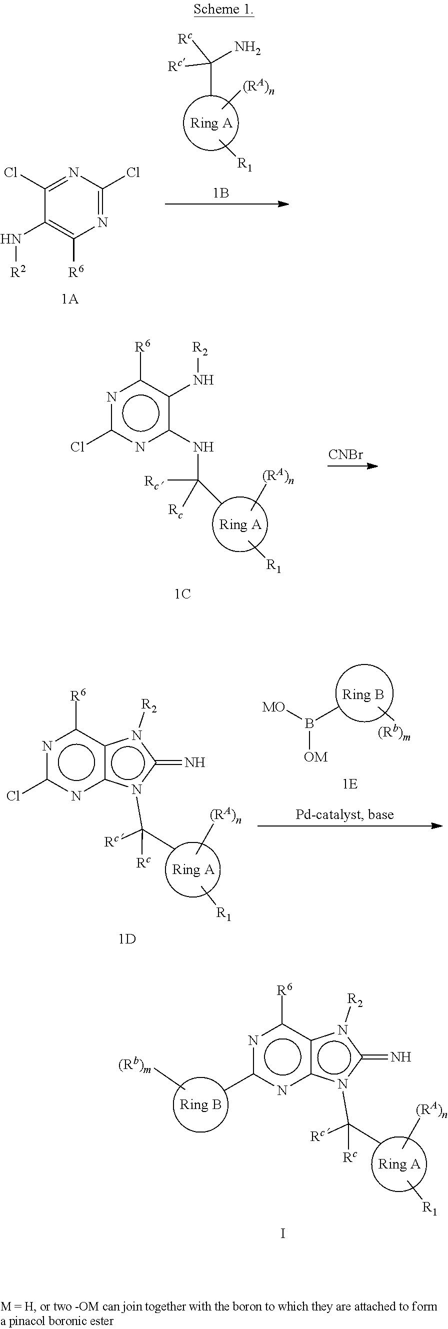

Dichloropyrimidines (1A) carrying the desired substituents R⁶ and NHR² are generally commercially available or they can prepared according to literature procedures using general methods well known in the art of synthetic organic chemistry. The dichloro substituted pyrimidine derivative (1A) is reacted with the desired amine building block (1B) in the presence of a base such as a tertiary amine like triethylamine or similar in an inert solvent such as DMF or THE or the like to provide the amino substituted pyrimidine derivative (IC). The reaction is typically carried out at a temperature from room temperature up to around 80-120° C. Treatment of the afforded amino substituted pyrimidine derivative with cyanogen bromide in a solvent like ethanol or similar then provides the bicyclic guanine derivative (1D). Introduction of Ring B is for instance by a palladium catalyzed reaction, e.g., a Suzuki reaction, with the suitable boronic acid or ester derivative (1E) in the presence of a base like a carbonate, such as sodium or cesium carbonate or similar, typically at elevated temperature, and provides the compound of formula (I). The heating in the palladium catalyst reaction is effected either by thermal heating or by microwave irradiation. Boronic acids (1E) are obtained e.g., from the corresponding bromide by treatment with a base such as BuLi or similar followed by reaction with triisopropylborate or the like. Amine building blocks (1B) for use as shown in Scheme 1 are prepared from commercially available starting materials according to literature procedures or as described in the General Schemes and Chemistry Examples & Intermediates sections herein below.

Dichloropyrimidines useful for the preparation of compounds disclosed herein are typically commercially available, or alternatively they can be prepared according to literature procedures using standard methods known to the person skilled in organic synthesis. For example, they can be prepared from an alkoxyamidine and a β-ketoester as illustrated in Scheme 2.

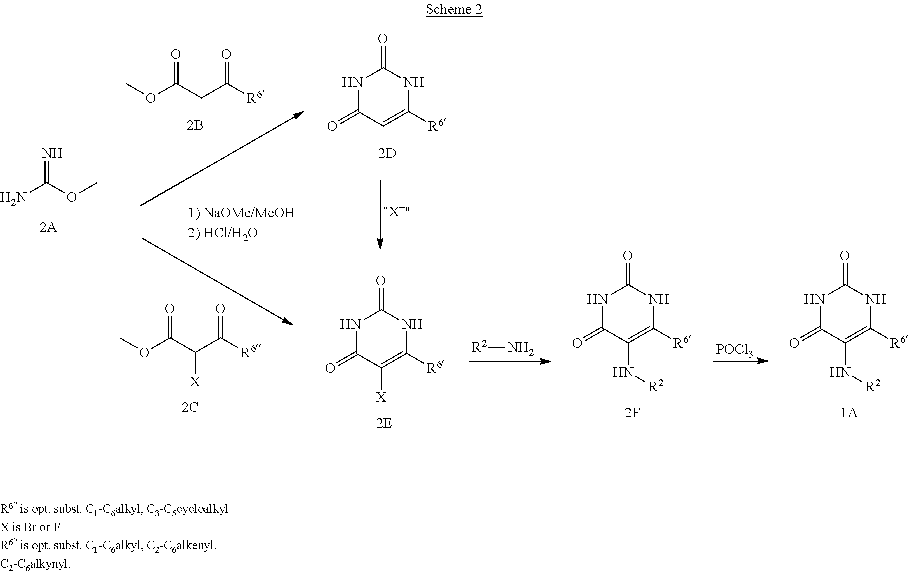

### Scheme 2

Condensation of methoxyamidine (2A) and a 0-ketoester carrying the desired group R⁶ (2B) or α-halo-β-ketoester (2C) and subsequent ring closure under basic conditions such as in methanolic sodium methoxide or equivalent followed by acidic demethoxylation provides pyrimidine derivative (2D) and (2E) respectively. Treatment of compound (2E) to electrophilic halogenation, for instance by treatment with bromine in acetic acid, or with an electrophilic fluorinating agent such as selectfluor or similar, provides the 5-halo compound (2E). The dichloropyrimidine (1A) is then obtained by way of a displacement reaction with a desired alkylamine R²NH₂ typically at an elevated temperature, followed by chlorination effected by treatment with phosphorus oxychloride in the presence of a base such as pyridine, trimethylamine or similar.

A pyrimidine derivative suitable for the preparation of compounds disclosed herein wherein R⁶ is C(═O)N(R^{a6})₂ can be prepared from the corresponding commercially available acid as shown in Scheme 3.

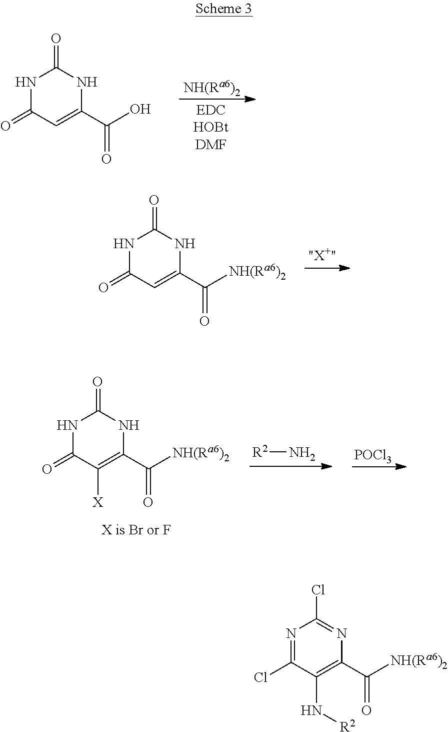

### Scheme 3

A pyrimidine derivative useful for the preparation of compounds disclosed herein wherein R⁶ is NHC(═O) R^{a6} can be prepared by acylation of commercially available amine as indicated in Scheme 4.

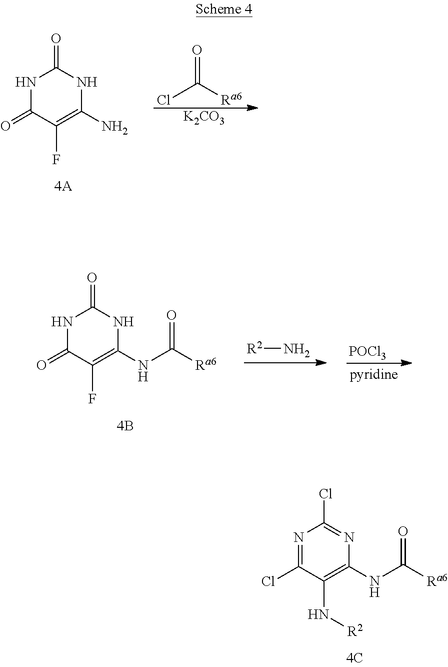

### Scheme 4

Acylation of the amine (4A) using the suitable acylating agent such as the acid halide R^{a6}C(═O)X wherein X typically is chloro, or acid anhydride R^{a6}OC(═O)OR^{a6} in the presence of a base such as trimethylamine, isopropylethylamine, pyridine, or a carbonate or the like, provides amine (4B). Subsequent reaction with the amine R²NH₂ followed by treatment with phosphorus oxychloride in the presence of a base like pyridine or similar provides the desired dichloro pyrimidine (4C).

A pyrimidine derivative useful for the preparation of compounds disclosed herein wherein R⁶ is CN can be prepared from commercially available acid as indicated in Scheme 5.

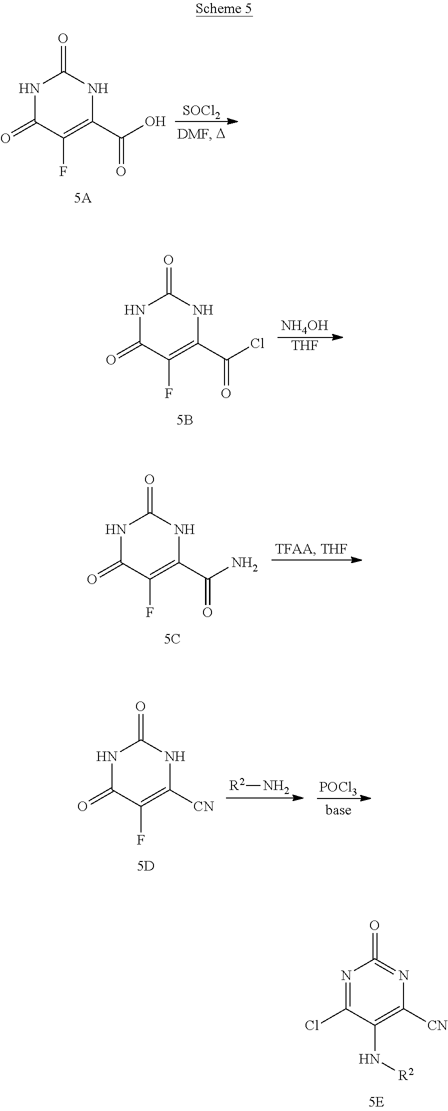

### Scheme 5

Conversion of commercially available acid (5A) to the corresponding acid chloride (5B) effected for instance by treatment with thionyl chloride or any other suitable conditions, followed by amination provides the primary amide (5C). The cyano function is then introduced by treatment with trifluoroacetic anhydride in THE or similar, thus providing cyano substituted pyrimidine derivative (5D). Alternatively, the acid (5A) can be converted to the corresponding cyano derivative (5D) using conditions in line with what those described in Open Journal of Med. Chem., 2014, 4, 39-60, i.e., by conversion of the acid moiety to the chloroactylamino moiety by treatment with chloroacetyl chloride followed by treatment with malonnitrile in the presence of a strong base. Introduction of the desired amine R₂NH₂ followed by conversion to the dichloro derivative as described above provides the desired cyano substituted pyrimidine derivative (5E).

A pyrimidine derivative useful for the preparation of compounds disclosed herein wherein R⁶ is N₃ can be prepared from commercially available amine as indicated in Scheme 6.

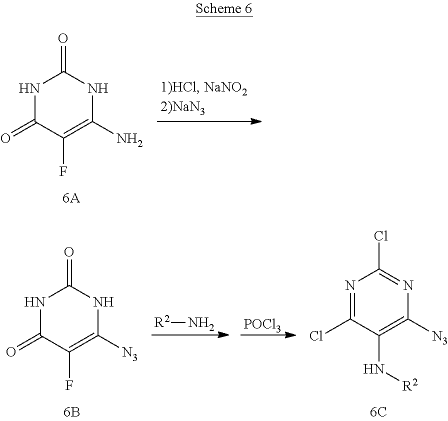

### Scheme 6

Diazotization of aminopyrimidine (6A) accomplished with sodium nitrite under acidic conditions such as in the presence of HCl or TFA or the like followed by coupling with sodium azide provides the azide substituted pyrimidine derivative (6B). Subsequent introduction of the desired amine R₂NH₂ followed by conversion to the dichloro derivative as described above provides the desired azido substituted pyrimidine derivative (6C). A pyrimidine derivative useful for the preparation of compounds disclosed herein wherein R⁶ is N(R^{6a})₂ can be prepared from commercially available dichloro substituted pyrimidine derivative as indicated in Scheme 7.

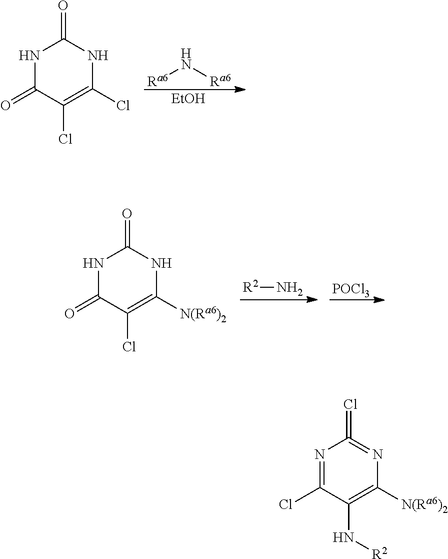

### Scheme 7

In an alternative approach to compounds disclosed herein, the desired substituent R⁶ is introduced at a later stage of the synthesis. A trichloro substituted pyrimidine derivative or equivalent is suitably used as starting material in this approach. The route is illustrated in Scheme 8.

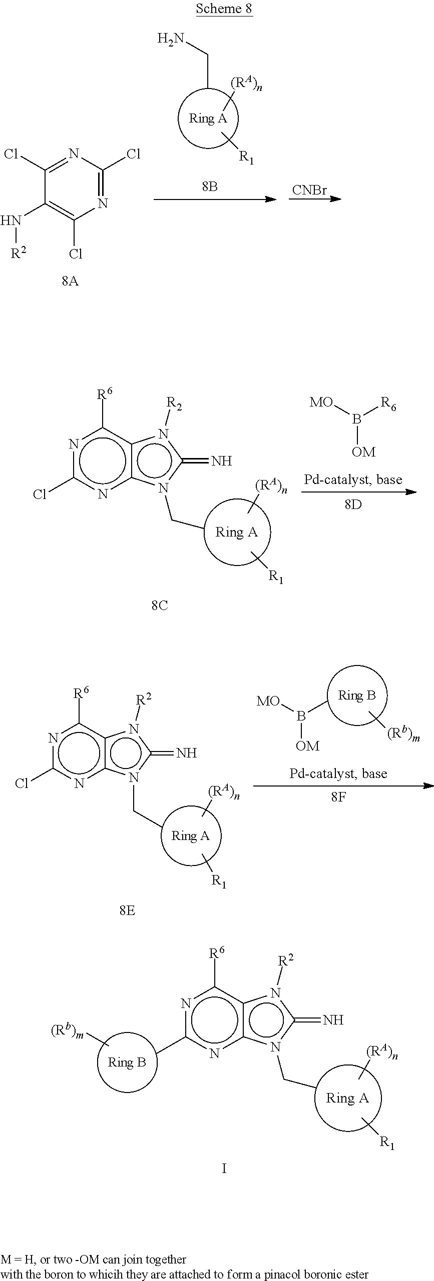

### Scheme 8

Reaction of trichloropyrimidine (8A) with the desired amine (8B) followed by ring closure as described in Scheme 1, provides dichloro substituted bicycle (8C). The substituent R⁶ can then be introduced by way of a palladium catalysed reaction such as a Suzuki coupling or similar, i.e., reaction with the appropriate boronic acid or ester of the group R⁶ (8D) in the presence of a base. The substituent Ring B is then introduced as described in Scheme 1, thus providing the compound of formula (I).

Compounds disclosed herein wherein R^{c} and R^{c′} form a C(═O) can be prepared as outlined in Scheme 10.

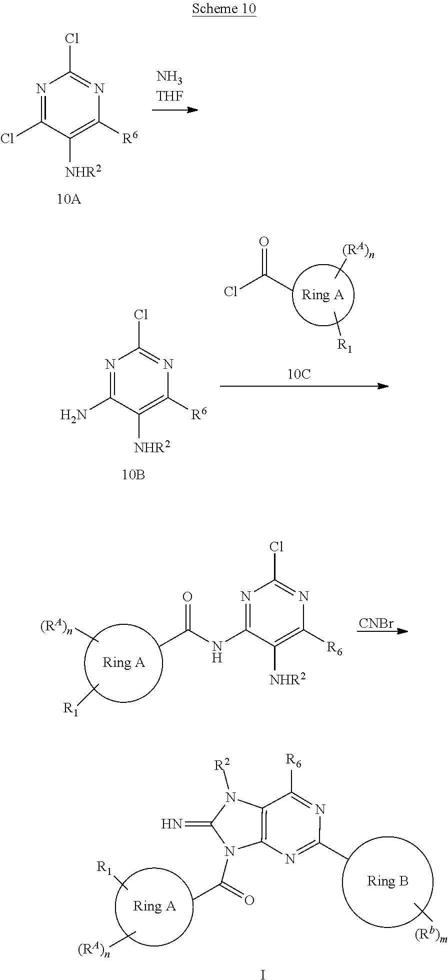

### Scheme 10

Treatment of dichloropyrimidine (10A) with ammonia in a solvent like THE or similar provides the corresponding amine (10B). The afforded amine is then reacted with an acid halide, typically acid chloride, of the desired Ring A-R¹ moiety (10C) provides the amide (10D). Ring closure accomplished by reaction with CNBr in ethanol or similar provides the compound of formula I wherein R^{c} and R^{c′} combine to form C(═O).

In an alternative approach to compounds disclosed herein wherein R^{c} and R^{c′} are both —F, the amide of Scheme 10 is fluorinated using a fluorinating agent like DAST or the like. This approach is briefly depicted in Scheme 12.

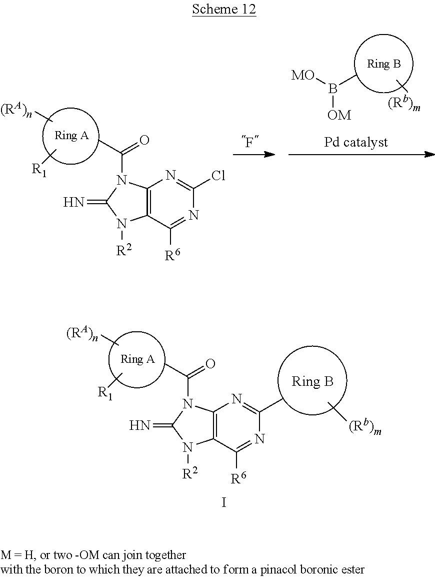

### Scheme 12

A Ring A amino moiety used in the above schemes wherein R¹ is a nitrogen containing heterocycle and the rings are linked to each other via an N-atom of R¹ can be prepared as depicted in Scheme 13.

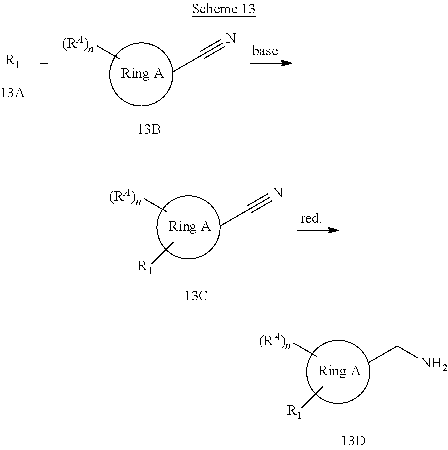

### Scheme 13

Reaction of R¹ (13A) with a fluoro- and cyano or cyanomethyl substituted derivative of Ring A (13B) in the presence of a base such as a carbonate, followed by reduction of the cyano group using any convenient reduction method, for instance treatment with LAH provides the amine (13D).

Certain compounds disclosed herein wherein Ring B is substituted with amino or alkoxy substituents can be prepared as shown in Scheme 14.

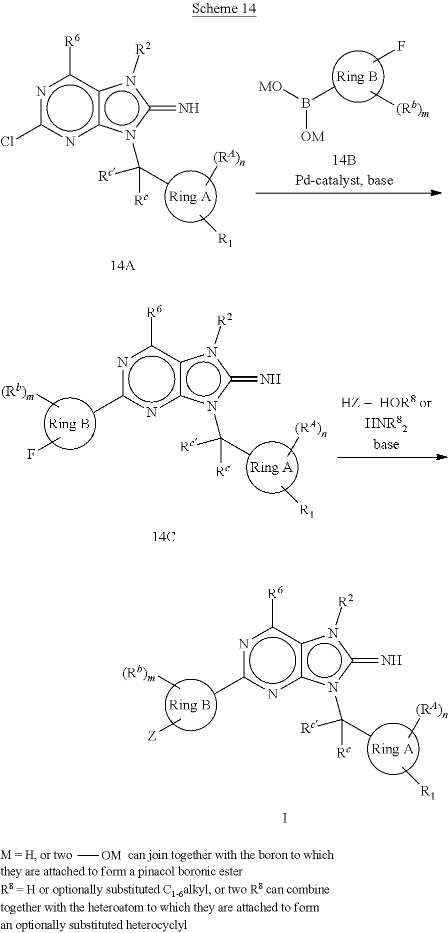

### Scheme 14

Reaction of 14A and fluoride-substituted 14B with base and a palladium catalyst (e.g., via a Suzuki reaction) results in coupled product 14C. Reaction of the fluoride moiety of 14C with an alcohol or amine respectively bearing one or two instances of R⁸ and base results in alkoxy or amino functionalized product of formula (I).

A Ring A amine used in the above schemes wherein Ring A is substituted with an alkyl- and trifluoro-substituted imidazo group, and the rings are linked to each other via the imidazo carbon as shown below (Scheme 15) can be prepared as depicted in Scheme 15.

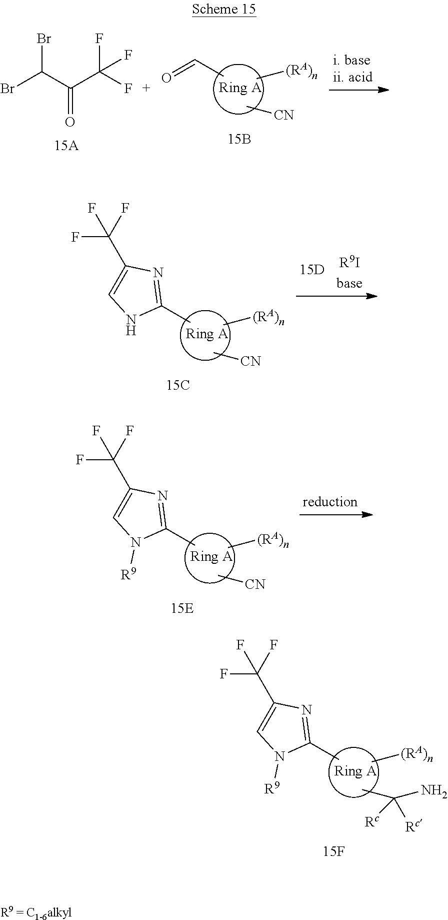

### Scheme 15

15A (3,3-dibromo-1,1,1-trifluoropropan-2-one) and 15B are reacted with base and then acid to produce product 15C. The imidazo substituent is alkylated by treatment with an alkyl iodide (15D) and base to afford 15E, and the cyano group of 15E is reduced (e.g., by treatment with lithium aluminum hydride) to afford amine product 15F.

In an alternative approach to compounds disclosed herein, the desired substituent R² is introduced at a later stage of the synthesis. This approach is briefly depicted in Scheme 16.

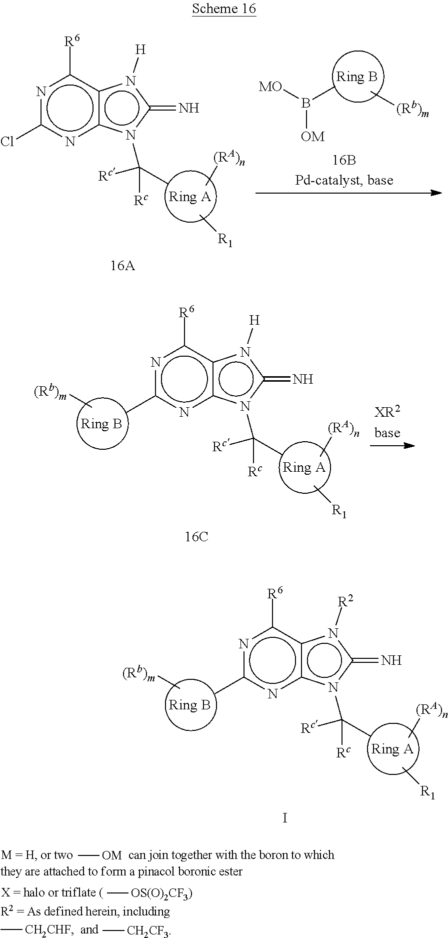

### Scheme 16

Reaction of 16A and the appropriately substituted boronic acid or ester derivative of 16B with base and a palladium catalyst (e.g., via a Suzuki reaction) results in coupled product 16C. Reaction of 16C with a halide or triflate functionalized R² in the presence of a base results in the functionalized product of formula (I).

In an alternative approach to compounds disclosed herein, the desired substituent R² is introduced at a later stage of the synthesis. This approach is briefly depicted in Scheme 17.

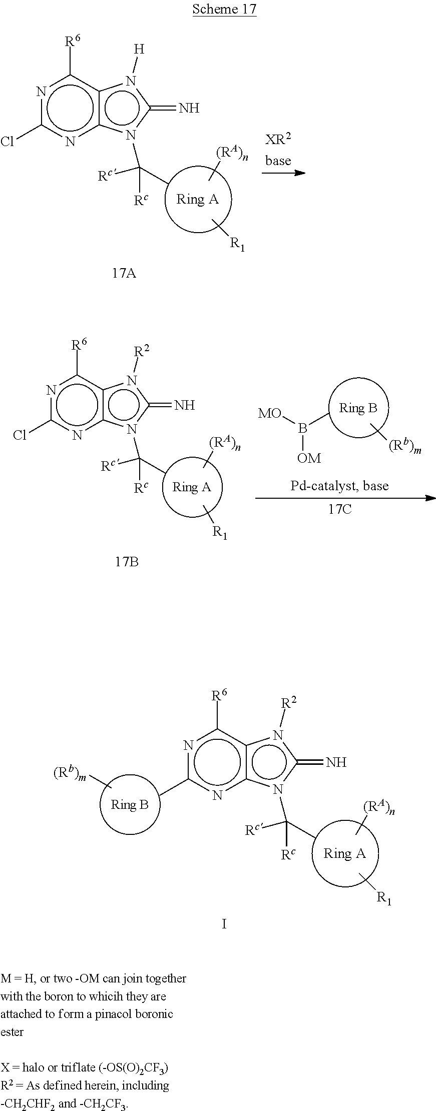

### Scheme 17

Reaction of 17A and the appropriate halide or triflate of R² in the presence of a base results in 17B. Then 17B reacted with the appropriate substituted boronic acid or ester derivative of 17C with base and a palladium catalyst (e.g., via a Suzuki reaction) results in formula (I).

### DETAILED DESCRIPTION OF THE EMBODIMENTS

Various embodiments of the compounds invention and intermediates therefore will now be illustrated by the following examples. The Examples are just intended to further illustrate the invention and are by no means limiting the scope disclosed herein.

### CHEMISTRY EXAMPLES & INTERMEDIATES

As is well known to a person skilled in the art, reactions are performed in an inert atmosphere (including but not limited to nitrogen and argon) where necessary to protect reaction components from air or moisture. Temperatures are given in degrees Celsius (° C.). Solution percentages and ratios express a volume to volume relationship, unless stated otherwise. The reactants used in the examples below may be obtained from commercial sources or they may be prepared from commercially available starting materials as described herein or by methods known in the art.

The compounds disclosed herein including intermediates are prepared as described in the Examples and in the general schemes herein. It will be apparent to a skilled person that analogous synthetic routes may be used, with appropriate modifications, to prepare the compounds disclosed herein as described herein. The progress of the reactions described herein were followed as appropriate by e.g., LC, GC or TLC, and as the skilled person will readily realize, reaction times and temperatures may be adjusted accordingly.

The compound names were generated by ChemDraw Ultra software, Cambridgesoft, version 12.0.2 and/or Scilligence 6.5.1.

### Intermediate 1

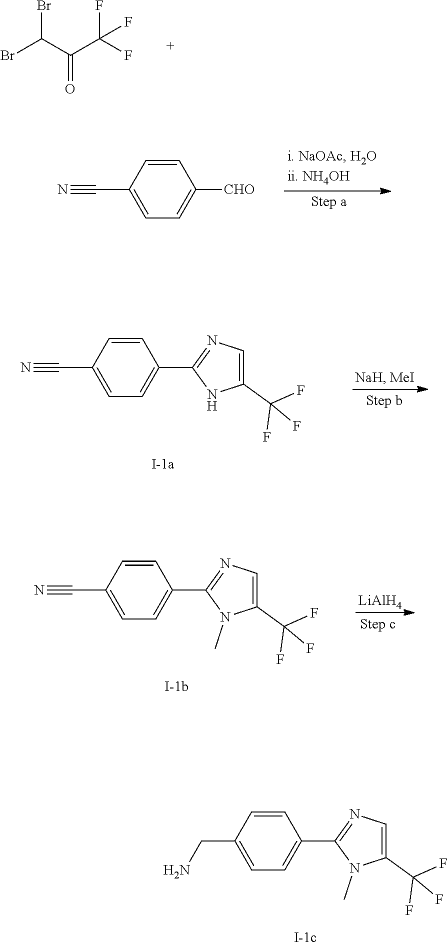 [SMILES: O=C(C(Br)Br)C(F)(F)F ; N#Cc1ccc(C=O)cc1 ; N#Cc1ccc(-c2ncc(C(F)(F)F)[nH]2)cc1 ; Cn1c(C(F)(F)F)cnc1-c1ccc(C#N)cc1 ; "LiAlH4" ; Cn1c(C(F)(F)F)cnc1-c1ccc(CN)cc1]

### Step a) 4-(5-(trifluoromethyl)-1H-imidazol-2-yl)benzonitrile (I-1a)

A mixture of sodium acetate (3.7 g, 44.9 mmol) and 3,3-dibromo-1,1,1-trifluoropropan-2-one (12 g, 44.03 mmol) in water (12 mL) was heated at 100° C. for 45 min, then was cooled to rt. The mixture was added to a solution of 4-formylbenzonitrile (5.8 g, 44.23 mmol) in MeOH (55 mL) followed by addition of 35% aq. NH₄OH (42 mL). The resulting reaction mixture was stirred at rt for 45 min, heated at 100° C. for 1 h, then concentrated. Water (50 mL) was added to the residue and the precipitated solid was filtered and dried, which gave the title compound (8 g) as a solid. LCMS (ES+) m/z 236.30 [M−H]⁻. The compound was taken to the next step without further purification.

### Step b) 4-(1-methyl-5-(trifluoromethyl)-1H-imidazol-2-yl)benzonitrile (I-1b)

NaH (60%, 1.35 g, 33.7 mmol) was added at 0° C. to a solution of compound I-1a (8 g, 33.7 mmol) in THE (80 ml). The mixture was stirred at 0° C. for 1 h, then CH₃I (2.1 mL, 33.7 mmol) was added at 0° C. and the stirring was then continued for 16 h. at rt. Ice cold water (40 mL) was added and the mixture was extracted with EtOAc (2×75 mL). The combined organic layers were washed with brine, dried (Na₂SO₄), filtered and concentrated. The crude compound was purified twice by column chromatography on silica gel and eluted with 15% EtOAc in pet ether, which gave the title compound (500 mg) as a solid. MS (ES+) 252.30 [M+H]⁺.

### Step c) (4-(1-methyl-5-(trifluoromethyl)-1H-imidazol-2-yl)phenyl)methanamine (I-1c)

LiAlH₄ (solid) (150 mg, 4.0 mmol) was added at 0° C. to a stirred solution of compound I-1b (500 mg, 2.0 mmol) in dry THE (25 mL). The resulting reaction mixture was stirred at rt until TLC indicated complete consumption of starting material (for 2 h), then the temperature was lowered to 0° C. and sodium sulfate solution (1 mL) was added. The cooling bath was removed and the resulting mixture was stirred at rt for 1 h, then filtered through Celite bed and the filtrate was concentrated under reduced pressure, which gave the title compound (500 mg, 90%) as a liquid. MS (ES+) 256.36 [M+H]⁺.

### Intermediate 2

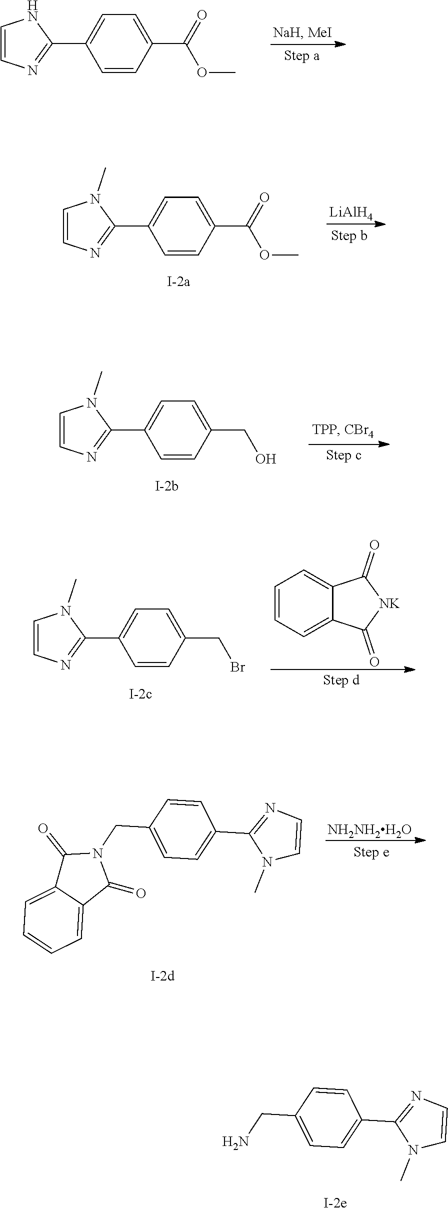

### Step a) methyl 4-(1-methyl-1H-imidazol-2-yl)benzoate (I-2a)

NaH (60%, 5.3 g, 132 mmol) was added at 0° C. to a solution of methyl 4-(1H-imidazol-2-yl)benzoate (18 g, 87.9 mmol) in DMF (300 mL). The mixture was stirred at rt for 15 min, then the temperature was lowered to 0° C. and CH₃I (6.6 mL, 105 mmol) was added. The mixture was stirred for 3 h. at rt, then ice cold water (400 mL) was added and the mixture was extracted with EtOAc (3×150 mL). The combined organic layers were washed with brine, dried (Na₂SO₄), filtered and concentrated under reduced pressure. The obtained crude was triturated with pet ether, which gave the title compound (11 g, 57%) as a solid. MS (ES+) 217.25 [M+H]⁺.

### Step b) (4-(1-methyl-1H-imidazol-2-yl)phenyl)methanol (I-2b)

To a suspension of LiAlH₄ (solid) (3.8 g, 100 mmol) in dry THE (300 mL) was added a solution of compound I-2a (11 g, 50.1 mmol) in THE (100 mL) at 0° C. The resulting reaction mixture was stirred at rt until TLC indicated complete consumption of starting material (4 h), then the temperature was lowered to 0° C. and sodium sulfate solution (12 mL) was added. The resulting mixture was stirred at rt for 10 min, then filtered through Celite bed and the filtrate was concentrated under reduced pressure, which gave the title compound (9 g, 92%) as a liquid. MS (ES+) 189.18 [M+H]⁺.

### Step c) 2-(4-(bromomethyl)phenyl)-1-methyl-1H-imidazole (I-2c)

CBr₄ (10.22 g, 30.81 mmol) and triphenylphosphine (8.1 g, 30.81 mmol) were added at 0° C. to a stirred solution of compound I-2b (4 g, 20.5 mmol) in DCM (200 mL). The mixture was stirred for 3 h at rt, then concentrated, which gave the crude title compound (20 g). MS (ES+) 253.21 [M+H]⁺. The compound was taken to the next step without further purification.

### Step d) 2-(4-(1-methyl-1H-imidazol-2-yl)benzyl)isoindoline-1,3-dione (I-2d)

To a stirred solution of compound I-2c (20 g, 7.60 mmol) in DMF (120 mL) was added potassium 1,3-dioxoisoindolin-2-ide (2.11 g, 11.41 mmol) at 0° C. The resulting reaction mixture was stirred at 80° C. for 8 h. Ice cold water (150 mL) was added and the mixture extracted with EtOAc (3×80 mL). The combined organic layers were washed with brine, dried (Na₂SO₄), filtered and concentrated under reduced pressure, which gave the crude title compound (12 g) as a solid. MS (ES+) 318.25 [M+H]⁺. The compound was taken to the next step without further purification.

### Step e) (4-(1-methyl-1H-imidazol-2-yl)phenyl)methanamine (I-2e)

Hydrazine hydrate (15 mL, 305 mmol) was added at 0° C. to a solution of I-2d (12 g, 15.3 mmol) in EtOH (250.0 mL). The resulting mixture was heated at 80° C. for 6 h, then cooled to rt. The precipitated solid was filtered and the filtrate was concentrated. Water (50 mL) was added to the residue and the precipitated solid was filtered, the filtrate was concentrated. The afforded crude was purified by column chromatography on neutral alumina, eluted with 3-5% MeOH/DCM, which gave the title compound (1.6 g) as a semi-solid. LCMS (ES+) m/z 188.21 [M+H]⁺.

The compound was used in next step without further purification.

### Intermediate 3

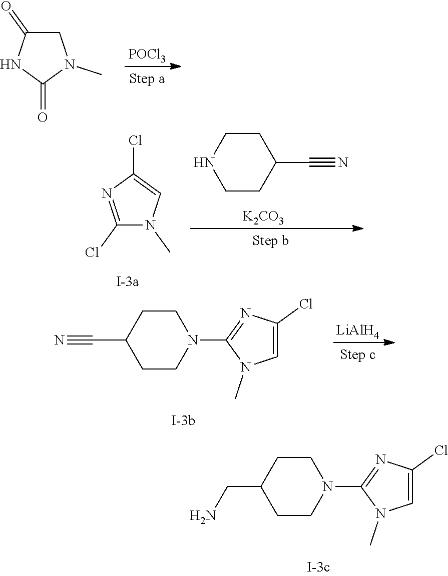

### Step a) 2,4-dichloro-1-methyl-1H-imidazole (I-3a)

Phosphorus oxychloride (200 mL, 2.14 mol) was added at rt to 1-methylimidazolidine-2,4-dione (20 g, 175.3 mmol). The mixture was refluxed for 4 h at 100° C., then cooled to rt and concentrated under reduced pressure. The residue was basified at 0° C. with saturated NaHCO₃ solution. The aqueous layer was extracted with EtOAc (2×250 mL). The combined organic layers were washed with saturated NaHCO₃ solution, brine, dried (Na₂SO₄), filtered and concentrated. The afforded crude was purified by column chromatography on silica gel, eluted with 17% EtOAc/pet ether, which gave the title compound (5.6 g, 20%) as a solid. LCMS (ES+) m/z 151.23 [M+H]⁺.

### Step b) 1-(4-chloro-1-methyl-1H-imidazol-2-yl)piperidine-4-carbonitrile (I-3b)

Potassium carbonate (17 g, 123 mmol) and piperidine-4-carbonitrile (27.14 g, 246.4 mmol) were added at rt to a stirred solution of compound I-3a (4 g, 24.6 mmol) in N-methyl-2-pyrrolidone (25 mL). The resulting reaction mixture was stirred at 180° C. for 24 h in steel bomb.

Water (50 mL) was added and the mixture was extracted with EtOAc (2×75 mL). The organic layer was washed with water, brine, dried (Na₂SO₄), filtered and concentrated. The crude compound was purified by column chromatography on silica gel and eluted with 35% EtOAc/pet ether, which gave the title compound (2 g, 34%) as a solid. LCMS (ES+) 225.36[M+H]⁺.

### Step c) (1-(4-chloro-1-methyl-1H-imidazol-2-yl)piperidin-4-yl)methanamine (I-3c)

LiAlH₄ (solid) (640 mg, 17.0 mmol) was added at 0° C. to a stirred solution of compound I-3b (2 g, 8.0 mmol) in dry THE (40 mL). The resulting reaction mixture was stirred at rt until TLC indicated complete consumption of starting material (2 h), then the temperature was lowered to 0° C. and sodium sulfate solution (12 mL) was added. The resulting mixture was stirred at rt for 1 h, then filtered through Celite bed and the filtrate was concentrated under reduced pressure, which gave the title compound (1.75 g, 81%) as a liquid. MS (ES+) 229.2 [M+H]⁺.

### Intermediate 4

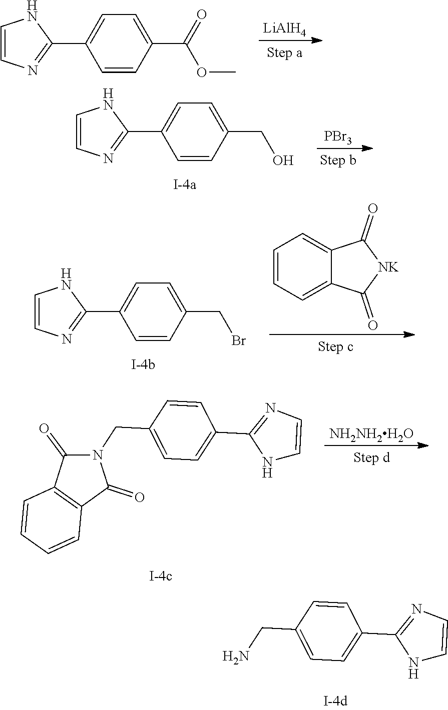

### Step a) (4-(1H-imidazol-2-yl)phenyl)methanol (I-4a)

To a stirred suspension of LiAlH₄ (solid) (9.84 g, 259.4 mmol) in dry THE (600 mL) was added methyl 4-(1H-imidazol-2-yl)benzoate (18 g, 86.5 mmol) at 0° C. The resulting reaction mixture was stirred at rt until TLC indicated complete consumption of starting material (16 h), then the temperature was lowered to 0° C. and sodium sulfate solution (12 mL) was added. The resulting mixture was stirred at rt for 10 min, then filtered through Celite bed and the filtrate was concentrated under reduced pressure, which gave the title compound (14 g, 90%) as a solid. MS (ES+) 175.17 [M+H]⁺.

### Step b) 2-(4-(bromomethyl)phenyl)-1H-imidazole (I-4b)

To a stirred solution of compound I-4a (14 g, 77.5 mmol) in DCM (700 mL) was added phosphorus tribromide (22.1 mL, 232.6 mmol) at 0° C. The resulting reaction mixture was stirred at rt for 16 h. The mixture was basified with saturated NaHCO₃ solution and the precipitated solid was filtered and dried, which gave the title compound (14 g, 55%) as a solid. MS (ES+) 239.20 [M+H]⁺.

### Step c) 2-(4-(1H-imidazol-2-yl)benzyl)isoindoline-1,3-dione (I-4c)

To a stirred solution of compound I-4b (14 g, 42.5 mmol) in DMF (300 mL) at 0° C. was added potassium 1,3-dioxoisoindolin-2-ide (8.3 g, 44.6 mmol) and heated at 80° C. for 8 h. Ice cold water (150 mL) was added and the mixture and extracted with EtOAc (3×150 mL). The combined organic layers were washed with brine, dried (Na₂SO₄), filtered and concentrated under reduced pressure. The crude compound was triturated with 15% DCM in diethyl ether, which gave the crude title compound (7.2 g) as a solid. MS (ES+) 304.24 [M+H]⁺. The compound was taken to the next step without further purification.

### Step d) (4-(1H-imidazol-2-yl)phenyl)methanamine (I-4d)

Hydrazine hydrate (14.1 mL, 286.6 mmol) was added at 0° C. to a solution of I-4c (7.2 g, 14.3 mmol) in EtOH (250.0 mL). The resulting mixture was stirred at 70° C. for 8 h, then cooled to rt. The precipitated solid was filtered and the filtrate was concentrated, which gave the crude title compound (3.5 g) as a semi-solid. LCMS (ES+) m/z 174.27 [M+H]⁺. The compound was used in next step without further purification.

### Intermediate 5

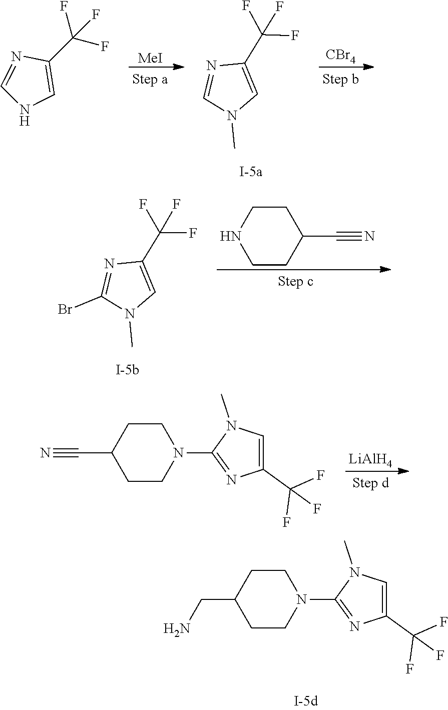

### Step a) 1-methyl-4-(trifluoromethyl)-1H-imidazole (1-5a)

NaH (60%, 5.9 g, 147 mmol) and CH₃I (5.5 mL, 88.2 mmol) were added at 0° C. to a solution of 4-(trifluoromethyl)-1H-imidazole (10 g, 73.5 mmol) in THE (250 ml). The mixture was stirred for 1 h at 0° C., then ice cold water (200 mL) was added and the mixture was extracted with EtOAc (2×250 mL). The combined organic layers were washed with brine, dried (Na₂SO₄), filtered and concentrated. The crude compound was purified by column chromatography on silica gel and eluted with 2-5% MeOH in DCM, which gave crude title compound as a mixture with inseparable isomer (8 g, 70:30 mixture) as a liquid. MS (ES+) 151.14 [M+H]⁺. The compound was taken to next step without further purification.

### Step b) 2-bromo-1-methyl-4-(trifluoromethyl)-1H-imidazole (1-5b)

n-BuLi (2.5M in hexane) (19.2 mL, 48.0 mmol) was added dropwise at −78° C. under argon to a solution of compound I-5a (8 g, 48.0 mmol) in dry THE (300 mL). The solution was stirred for 15 min at −78° C., then a solution of CBr₄ (19.25 g, 58 mmol) in THE (100 mL) was added at −78° C. and stirred at that temperature for 2 h followed by 1 h. at rt. To the reaction mixture saturated ammonium chloride solution was added and the mixture was extracted with EtOAc (4×100 mL). The combined organic layers were washed with brine, dried (Na₂SO₄), filtered and concentrated. The crude compound was purified by column chromatography on silica gel and eluted with 30-70% EtOAc in pet ether, which gave the title compound (2.5 g, 20%) as a liquid. MS (ES+) 229.12 [M+H]⁺.

### Step c) 1-(1-methyl-4-(trifluoromethyl)-1H-imidazol-2-yl)piperidine-4-carbonitrile (1-5c)

A mixture of potassium carbonate (3.31 g, 24 mmol), piperidine-4-carbonitrile (14.3 mL, 128 mmol) and compound I-5b (2.5 g, 11.0 mmol) was heated at 150° C. for 36 h in a sealed tube, then ice cold water (50 mL) was added and the mixture was extracted with EtOAc (3×75 mL). The combined organic layers were washed with brine, dried (Na₂SO₄), filtered and concentrated. The crude compound was purified by column chromatography on silica gel and eluted with 30% EtOAc in pet ether, which gave the title compound (1.8 g) as a semi-solid. MS (ES+) 259.23 [M+H]⁺.

### Step d) (1-(1-methyl-4-(trifluoromethyl)-1H-imidazol-2-yl)piperidin-4-yl)methanamine (I-5d)

LiAlH₄ (solid) (470 mg, 12.41 mmol) was added at 0° C. to a stirred solution of compound I-5c (1.8 g, 6.20 mmol) in dry THE (60 mL). The resulting reaction mixture was stirred at 0° C. until TLC indicated complete consumption of starting material (2 h), then the temperature was lowered to 0° C. and sodium sulfate solution (5 mL) was added. The resulting mixture was stirred at rt for 15 min, then filtered through Celite bed and concentrated under reduced pressure, which gave the crude title compound (1.6 g) as a semi-solid. MS (ES+) 263.26[M+H]⁺.

### Intermediate 6

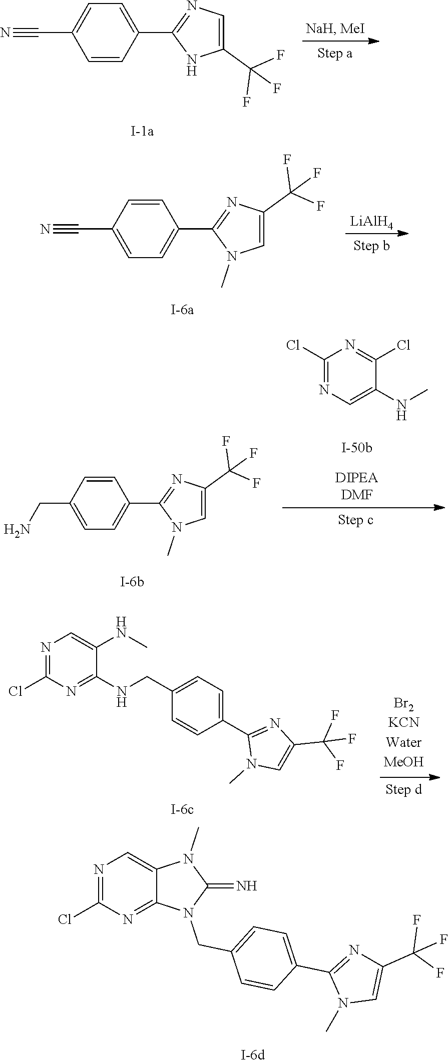

### Step a) 4-(1-methyl-5-(trifluoromethyl)-1H-imidazol-2-yl)benzonitrile (I-6a)

NaH (60%, 4.34 g, 108.5 mmol) was added at 0° C. to a solution of compound I-1a (31 g, 108.5 mmol) in THE (320 ml) and stirred at 0° C. for 1 h. CH₃I (6.8 mL, 108.5 mmol) was added at 0° C. and the mixture was stirred for 16 h. at rt. Ice cold water (400 mL) was added and the mixture was extracted with EtOAc (2×250 mL). The combined organic layers were washed with brine, dried (Na₂SO₄), filtered and concentrated. The crude compound was purified by column chromatography on silica gel and eluted with 10-20% EtOAc in pet ether, which gave the title compound (12 g, 42%) as a solid. MS (ES+) 252.09 [M+H]⁺.

### Step b) (4-(1-methyl-5-(trifluoromethyl)-1H-imidazol-2-yl)phenyl)methanamine (I-6b)

LiAlH₄ (solid) (3.5 g, 91.72 mmol) was added at 0° C. to a stirred solution of compound I-6a (12 g, 45.90 mmol) in dry THE (250 mL). The resulting reaction mixture was stirred at rt until TLC indicated complete consumption of starting material (2 h), then the temperature was lowered to 0° C. and sodium sulfate solution (12 mL) was added. The resulting mixture was stirred at rt for 1 h, then filtered through Celite bed and the filtrate was concentrated under reduced pressure, which gave the title compound (10 g, 80%) as a liquid. MS (ES+) 256.20 [M+H]⁺.

### Step c) 2-chloro-N5-methyl-N4-[[4-[1-methyl-4-(trifluoromethyl)imidazol-2-yl]phenyl]methyl]pyrimidine-4,5-diamine

To a stirred solution of [4-[1-methyl-4-(trifluoromethyl)imidazol-2-yl]phenyl]methanamine (4.30 g, 11.8 mmol) in DMF (20 mL) DIPEA (4.57 g, 35.4 mmol, 6.16 mL) and 2,4-dichloro-N-methyl-pyrimidin-5-amine (2.73 g, 15.3 mmol) were added. The mixture was stirred at 100° C. for 18 hr then cooled to r.t. The reaction mixture was diluted with water (10 mL) and extracted with EtOAc (3×20 mL). The combined organic extracts were washed with brine (30 mL), dried over Na₂SO₄, filtered and concentrated in vacuo to give 2-chloro-N₅-methyl-N4-[[4-[1-methyl-4-(trifluoromethyl)imidazol-2-yl]phenyl]methyl]pyrimidine-4,5-diamine (4.9 g, crude, 77% purity by LCMS) as a red oil which was used in the next step without further purification.

LCMS(ESI): [M+H]⁺ m/z: calcd 397.13; found 397.2; Rt=1.09.

### Step d) 2-chloro-7-methyl-9-(4-(1-methyl-4-(trifluoromethyl)-1H-imidazol-2-yl)benzyl)-7H-purin-8(9H)-imine

To a stirred solution of potassium cyanide (2.85 g, 43.8 mmol) in water (20 mL) a solution of molecular bromine (6.99 g, 43.8 mmol) in MeOH (250 mL) was added at r.t. The reaction mixture was stirred for 1 hr. Then 2-chloro-N5-methyl-N4-[[4-[1-methyl-4-(trifluoromethyl)imidazol-2-yl]phenyl]methyl]pyrimidine-4,5-diamine (4.51 g, 77% purity, 8.75 mmol) was added. The mixture was stirred for 40 hr. at r.t. The reaction mixture was diluted with EtOAc (200 mL) then potassium carbonate (10 g) was added. The obtained mixture was stirred for 15 min. The organic phase was separated; the aqueous layer was extracted with EtOAc (2×100 mL). The combined organic phase was washed with brine (3×100 mL), dried over anhydrous Na₂SO₄ and concentrated under reduce pressure. The residue was subjected to flash-column chromatography (SiO₂; ACN-MeOH) to yield 2-chloro-7-methyl-9-(4-(1-methyl-4-(trifluoromethyl)-1H-imidazol-2-yl)benzyl)-7H-purin-8(9H)-imine (2.60 g, 6.16 mmol, overall yield from [4-[1-methyl-4-(trifluoromethyl)imidazol-2-yl]phenyl]methanamine is 56.76%) as a light yellow solid which can be used in the next steps without further purification.

¹H NMR (400 MHz, DMSO) δ 3.33 (s, 3H), 3.76 (s, 3H), 5.13 (s, 2H), 6.85 (br, 1H), 7.47 (d, 2H), 7.68 (d, 2H), 7.92 (s, 1H), 8.01 (s, 1H).

LCMS(ESI): [M+H]⁺ m/z: calcd 422.12; found 422.0; Rt=0.91.

### Intermediate 7

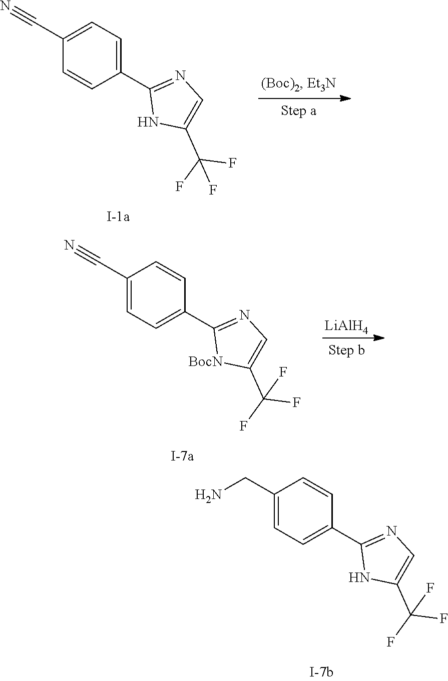 [SMILES: N#Cc1ccc(-c2ncc(C(F)(F)F)[nH]2)cc1 ; CC(C)(C)OC(=O)n1c(C(F)(F)F)cnc1-c1ccc(C#N)cc1 ; "LiAlH4" ; NCc1ccc(-c2ncc(C(F)(F)F)[nH]2)cc1]

### Step a) tert-butyl 2-(4-cyanophenyl)-5-(trifluoromethyl)-1H-imidazole-1-carboxylate (I-7a)

Et₃N (1.73 mL, 12.4 mmol) was added at 0° C. to a solution of compound I-1a (1.5 g, 6 mmol) in DCM (10 mL), then BOC anhydride (1.7 mL, 7.4 mmol) was added at 0° C. The reaction mixture was stirred for 16 h at rt, then diluted with DCM. The organic layer was washed with water, brine, dried (Na₂SO₄) and concentrated. The crude product was purified by column chromatography on silica gel eluted with a gradient of 15-20% EtOAc in pet ether which gave the title compound (1 g, 34%) as a solid. LCMS (ES+) m/z 339.21 [M+H]⁺.

### Step b) (4-(5-(trifluoromethyl)-1H-imidazol-2-yl)phenyl)methanamine (I-7b)

LiAlH₄ (solid) (162 mg, 4.3 mmol) was added at 0° C. to a stirred solution of compound I-7a (1 g, 2.12 mmol) in dry THE (15 mL). The resulting reaction mixture was stirred at rt for 2 h, then sodium sulfate solution was added and the resulting mixture was extracted with EtOAc. The combined organic layers were dried (Na₂SO₄), filtered and concentrated under reduced pressure, which gave the title compound (600 mg, 96%) as a semi solid. MS (ES+) 240.35 [M−H]⁻. The compound was taken to next step without further purification.

### Intermediate 8

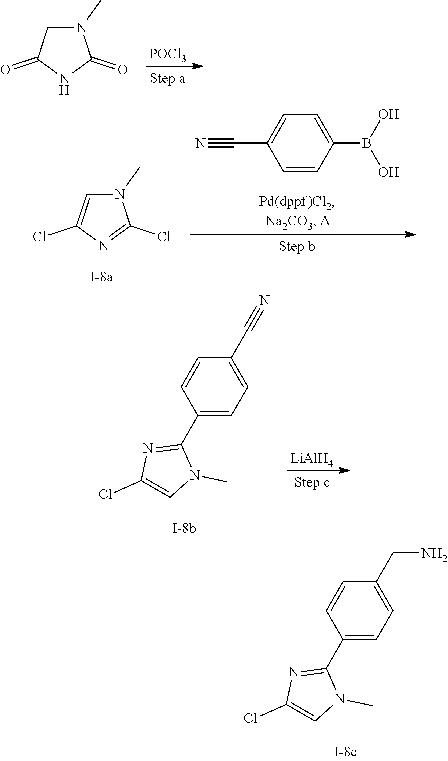

### Step a) 2,4-dichloro-1-methyl-1H-imidazole (I-8a)

Phosphorus oxychloride (200 mL, 2139 mmol) was added at rt to 1-methylimidazolidine-2,4-dione (20 g, 175.3 mmol). The mixture was refluxed for 4 h at 100° C., then cooled to rt and concentrated under reduced pressure. Ice cold water was added to the residue was basified with saturated NaHCO₃ solution at 0° C. The aqueous layer was extracted with EtOAc. The combined organic layers were washed with saturated brine, dried (Na₂SO₄), filtered and concentrated. The crude product was purified by column chromatography on silica gel eluted with a gradient of 17% EtOAc in pet ether which gave the title compound (5.7 g, 20%) as a solid. LCMS (ES+) m/z 151.02 [M+H]⁺.

### Step b) 4-(4-chloro-1-methyl-1H-imidazol-2-yl)benzonitrile (I-8b)

Sodium carbonate (1.75 g, 16.6 mmol) was added to a stirred solution of compound I-8a (500 mg, 3.0 mmol) and (4-cyanophenyl)boronic acid (975 mg, 6.62 mmol) in 1,4-dioxane (6 mL) and water (2 mL) in a sealed tube. The reaction mixture was degassed by bubbling with argon for 10 min then Pd(dppf)Cl₂·DCM, (1.35 g, 2.0 mmol) was added and the reaction mixture was stirred at 100° C. for 16 h in a sealed tube. The reaction mixture was diluted with water, filtered through the celite bed, extracted with EtOAc and the combined organic layers were washed with brine, dried (Na₂SO₄), filtered and concentrated under reduced pressure. The crude product was purified by column chromatography on silica gel eluted with a gradient of 20-30% EtOAc in pet ether which gave the title compound (400 mg, 54%) as a solid. LCMS (ES+) m/z 218.31 [M+H]⁺.

### Step c) (4-(4-chloro-1-methyl-1H-imidazol-2-yl)phenyl)methanamine (I-8c)

LiAlH₄ (solid) (227 mg, 6.0 mmol) was added at 0° C. to a stirred solution of compound I-8b (650 mg, 3.0 mmol) in dry THE (15 mL). The resulting reaction mixture was stirred at rt for 2 h, then sodium sulfate solution was added and the resulting mixture was extracted with EtOAc. The combined organic layers were dried (Na₂SO₄), filtered and concentrated under reduced pressure, which gave the title compound (700 mg, 99%) as a semi solid. MS (ES+) 222.36 [M+H]⁺.

### Intermediate 9

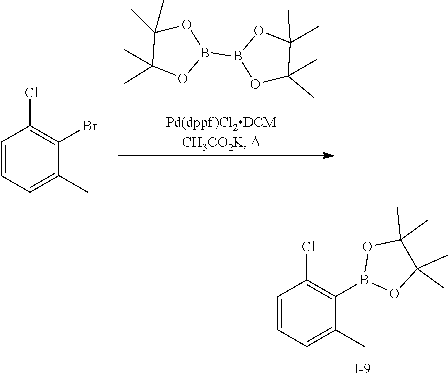

### Step a) 2-(2-chloro-6-methylphenyl)-4,4,5,5-tetramethyl-1,3,2-dioxaborolane (I-9)

Potassium acetate (717 mg, 7.3 mmol) was added to a solution of 4,4,4′,4′,5,5,5′,5′-octamethyl-2,2′-bi(1,3,2-dioxaborolane) (741 mg, 2.92 mmol) in DMF (20 mL). The resulting mixture was de-gassed for 15 min with argon, then bis(triphenylphosphine)palladium(II) dichloride (99 mg, 0.12 mmol) and 2-bromo-1-chloro-3-methylbenzene (500 mg, 2.43 mmol) were added, the mixture was de-gassed for 5 min then stirred at 110° C. for 16 h in a sealed tube. The reaction mixture was diluted with water, and extracted with EtOAc (3×25 mL). The organic layer was washed with brine (15 mL), dried (Na₂SO₄) and concentrated under reduced pressure, which gave the crude title compound (450 mg). MS (ES+) 253.27 [M+H]⁺.

### Intermediate 10

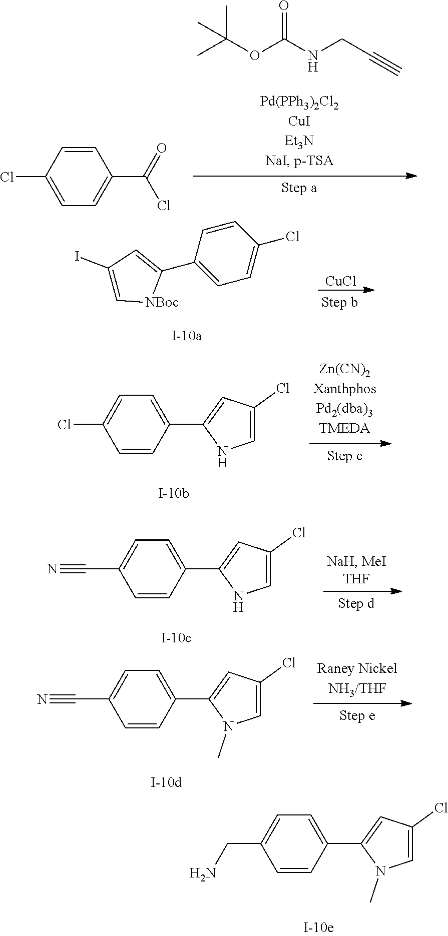

### Step a) tert-butyl 2-(4-chlorophenyl)-4-iodo-1H-pyrrole-1-carboxylate (I-10a)

A mixture of Pd(PPh₃)₂Cl₂ (400 mg, 0.6 mmol) and CuI (110 mg, 0.06 mol) was heated with hot air gun in a sealed tube, cooled to rt and THF (40 mL) was added and de-gassed. Et₃N (2.9 g, 3.0 mmol) was added, followed by addition of 4-Chloro benzoyl chloride (5 g, 28.6 mmol) and tert-butyl prop-2-yn-1-ylcarbamate (4.43 g, 28.6 mmol) while degassing the reaction and was stirred at rt for 1 h. NaI (21.41 g, 14 mmol) was then added to the reaction, followed by p-toluene sulfonic acid (10.87 g, 6.0 mol) and t-BuOH (20 mL). The reaction was then stirred at rt for 2 h. The reaction mixture was diluted with water and extracted with EtOAc. The combined organic layers were dried (Na₂SO₄), filtered and concentrated under reduced. The crude product was purified by column chromatography on silica gel eluted with a gradient of 10-25% EtOAc in pet ether which gave the title compound (5 g, 41%) as a semi-solid. LCMS (ES+) m/z 402.13 [M−H]⁻.

### Step b) 4-chloro-2-(4-chlorophenyl)-1H-pyrrole (I-10b)

CuCl (12.3 g, 0.12 mol) was added to a stirred solution of compound I-10a (5 g, 0.012 mol) in DMF (50 mL) and stirred at 110° C. for 16 h in a sealed tube. The reaction mixture was filtered through the celite bed, the filtrate was diluted with water and extracted with 10% MeOH in DCM. The combined organic layers were dried (Na₂SO₄), filtered and concentrated under reduced pressure. The crude product was purified by column chromatography on silica gel eluted with a gradient of 15-25% EtOAc in pet ether which gave the title compound (2.2 g, 43%) as a semi-solid. LCMS (ES+) m/z 212.20 [M+H]⁺.

### Step c) 4-(4-chloro-1H-pyrrol-2-yl)benzonitrile (I-10c)

To a de-gassed stirred solution of compound I-10b (2.2 g, 10.4 mmol) in DMF (15 mL) was added TMEDA (603 mg, 5.2 mmol), Zn(CN)₂ (731 mg, 6.2 mmol) followed by Pd₂(dba)₃ (475 mg, 0.52 mmol) and Xanthphos (600 mg, 1.04 mmol) after de-gassing for 5 min the reaction was heated to 180° C. for 2 h in a microwave. The reaction mixture was diluted with water and extracted with EtOAc. The combined organic layers were dried (Na₂SO₄), filtered and concentrated under reduced. The crude product was purified by column chromatography on silica gel eluted with a gradient of 10-15% EtOAc in pet ether which gave the title compound (1.0 g, 28%) as a solid. LCMS (ES−) m/z 201.21 [M−H]⁻.

### Step d) 4-(4-chloro-1-methyl-1H-pyrrol-2-yl)benzonitrile (I-10d)

NaH (60%, 395 mg, 9.9 mmol) was added at 0° C. to a solution of compound I-10c (1 g, 5.0 mmol) in DMF (25 mL). The mixture was stirred for 15 min at 0° C., then CH₃I (0.37 mL, 6.0 mmol) and stirred for 2 h. at rt. The reaction was quenched by adding water and the mixture was extracted with EtOAc. The combined organic layers were washed with brine, dried (Na₂SO₄), filtered and concentrated under reduced pressure. The crude compound was purified by column chromatography on silica gel and eluted with 15-25% EtOAc in pet ether, which gave the title compound (900 mg, 50%) as a liquid. MS (ES+) 217.22 [M+H]⁺.

### Step e) (4-(4-chloro-1-methyl-1H-pyrrol-2-yl)phenyl)methanamine (I-10e)

Compound I-10d (900 mg, 4.2 mmol) was added to a solution of Raney nickel (731 mg, 12 mmol) and 7M NH₃ in THF (6 mL) in EtOH (10 mL). The reaction mixture was stirred under hydrogen balloon at rt for 6 h, then the reaction mixture was filtered through Celite bed, washed with 10% MeOH in DCM and the filtrate was concentrated under reduced pressure which gave the title compound (800 mg) as a liquid. The compound was taken to next step without further purification.

### Intermediate 11

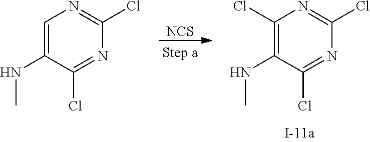

### Step a) 2,4,6-trichloro-N-methylpyrimidin-5-amine (I-11a)

To a stirred solution of 2,4-dichloro-N-methylpyrimidin-5-amine (1 g, 5.62 mmol) in THF (50 mL) was added NCS (1.5 g, 11.23 mmol) at rt. The resulting reaction mixture was stirred at rt for 16 h, then water (100 mL) was added and extracted with EtOAc (3×150 mL). The combined organic layers were washed with brine (200 mL), dried (Na₂SO₄), filtered and concentrated under reduced pressure. The crude compound was purified by column chromatography on silica gel and eluted with 20% EtOAc in pet ether, which gave the title compound (800 mg, 67%) as a solid. MS (ES+) 214.16 [M+H]⁺.

### Intermediate 12

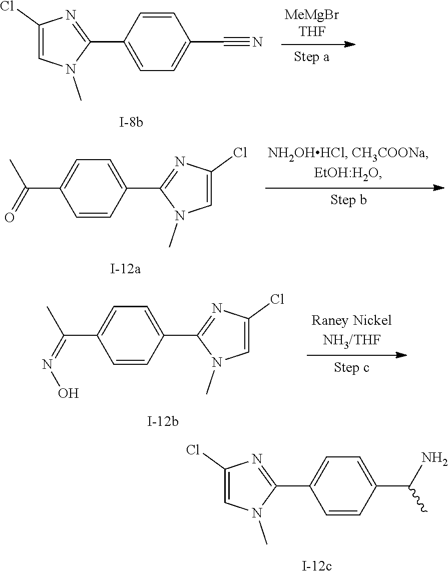 [SMILES: Cn1cc(Cl)nc1-c1ccc(C#N)cc1 ; CC(=O)c1ccc(-c2nc(Cl)cn2C)cc1 ; C/C(=N/O)c1ccc(-c2nc(Cl)cn2C)cc1 ; CC(N)c1ccc(-c2nc(Cl)cn2C)cc1]

### Step a) 1-(4-(4-chloro-1-methyl-1H-imidazol-2-yl)phenyl)ethan-1-one (I-12a)

MeMgBr (1M in THF) (83 mL, 8.3 mmol) was added at 0° C. to a stirred solution of compound I-8b (4.1 g, 8 mmol) and CuI (1.3 g, 7 mmol) in THF (100 mL). The resulting mixture was stirred at rt for 0.5 h, then the reaction was quenched with ammonium chloride solution (˜50 mL) at 0° C. and extracted with EtOAc (2×25 mL). The combined organic layers were dried (Na₂SO₄), filtered and concentrated under reduced pressure. The crude compound was purified by column chromatography on silica gel and eluted with 33% EtOAc in pet ether, which gave the title compound (3.2 g, 98%) as a solid. MS (ES+) 235.23 [M+H]⁺.

### Step b) (Z)-1-(4-(4-chloro-1-methyl-1H-imidazol-2-yl)phenyl)ethan-1-one oxime (I-12b)

Sodium acetate (2.22 g, 27 mmol) was added to a stirred solution of compound I-12a (3.2 g, 14 mmol) in EtOH (50 mL) and water (25 mL), followed by addition of hydroxylamine hydrochloride (1.9 g, 27 mmol). The resulting mixture was heated at 85° C. for 4 h, then concentrated under reduced pressure. Water (15 mL) was added to the residue and stirred for 15 min, the precipitated solid was filtered and dried, which gave the crude title compound (3.0 g, 88%) as a solid. LCMS (ES+) m/z 250.19 [M+H]⁺.

### Step c) 1-(4-(4-chloro-1-methyl-1H-imidazol-2-yl)phenyl)ethan-1-amine (I-12c)

Raney nickel (450 mg, 7.6 mmol) was added to a solution of compound I-12b (1.0 g, 4.0 mmol) in EtOH (70 mL), then 7M NH₃ in MeOH (1.0 mL) The reaction mixture was stirred in a Parr shaker at 60 psi at rt for 6 h, then the reaction mixture was filtered through Celite bed, washed with MeOH (2×25 mL) and the filtrate was concentrated under reduced pressure, which gave the title compound (600 mg, 53%) as a semi solid. MS (ES+) 236.22 [M+H]⁺.

### Intermediate 13

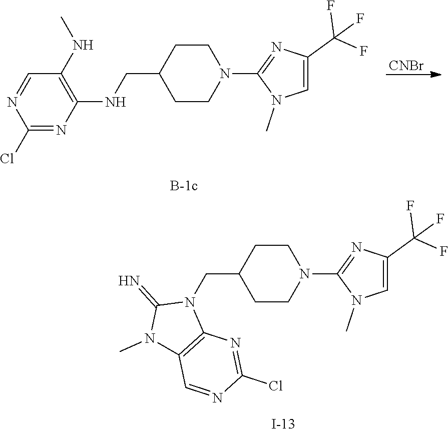

### 2-chloro-7-methyl-9-((1-(1-methyl-4-(trifluoromethyl)-1H-imidazol-2-yl)piperidin-4-yl)methyl)-7H-purin-8(9H)-imine (I-13)

Cyanogen bromide (386 mg, 3.64 mmol) was added at 0° C. to a stirred solution of compound B-1c (600 mg, 0.73 mmol) in EtOH (15 mL). The resulting mixture was stirred at 80° C. for 16 h, then concentrated. The residue was dissolved in water (basified by adding saturated NaHCO₃ solution) and extracted with 10% MeOH/DCM. The combined organic layers were dried (Na₂SO₄), filtered and concentrated under reduced pressure. The afforded crude compound was combined with another batch and purified by column chromatography on silica gel, eluted with 4-5% MeOH/DCM. The obtained impure compound was further purified by prep HPLC on an X-Select C18 (19×150) mm 5μ column using a gradient of 10 mM NH₄OAc in H₂O:MeCN as mobile phase. The impure product was further purified by prep HPLC on an X-Select C18 (19×150) mm 5μ column using a gradient of 10 mM NH₄OAc in H₂O:MeCN as mobile phase, which gave the title compound (50 mg, 15%) as a solid. LCMS (ES+) m/z 429.43 [M+H]⁺.

¹H NMR (500 MHz, DMSO): δ 7.94 (d, J=17.5 Hz, 1H), 7.52 (d, J=1.4 Hz, 1H), 6.66 (d, J=63.4 Hz, 1H), 3.77 (d, J=18.6 Hz, 2H), 3.48 (s, 3H), 3.26 (q, J=9.7 Hz, 3H), 2.66 (q, J=8.3 Hz, 2H), 1.99 (q, J=32.2 Hz, 1H), 1.66 (d, J=10.9 Hz, 2H), 1.42 (m, J=5.5 Hz, 2H).

### Intermediate 14

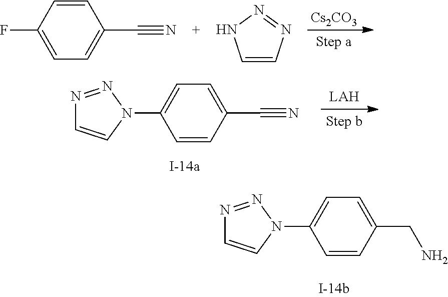

### Step a) 4-(1H-1,2,3-Triazol-1-yl)benzonitrile (I-14a)

Cesium carbonate (80.7 g) and 1H-1,2,3-triazole (5.7 g) were added at rt to a stirred solution of 4-fluorobenzonitrile (10.0 g) in DMF (120.0 mL). The resulting reaction mixture was stirred under N₂ at 80° C. for 3 h, then diluted with water (500 mL), stirred for 15 min and filtered. The crude was purified by column chromatography on silica gel eluted with 70% EtOAc in pet ether, which gave the title compound (5.0 g, 36%) as a solid. LCMS (ES+) m/z 171.04 [M+H]⁺.

### Step b) (4-(1H-1,2,3-Triazol-1-yl)phenyl)methanamine (I-14b)

Lithium aluminium hydride (solid) (5.57 g) was added at −10° C. over a period of 20 minutes to a stirred solution of I-14a (5.0 g) in dry THE (250 mL). The resulting reaction mixture was stirred at 0° C. until TLC indicated complete consumption of starting material (1 h), then 20% NaOH solution (50 mL) was added. The mixture was filtered through Celite and concentrated under reduced pressure. The obtained semi solid was triturated with diethyl ether (50 mL) and dried which gave the title compound (5.0 g, 84%) as a solid.

### Intermediate 15

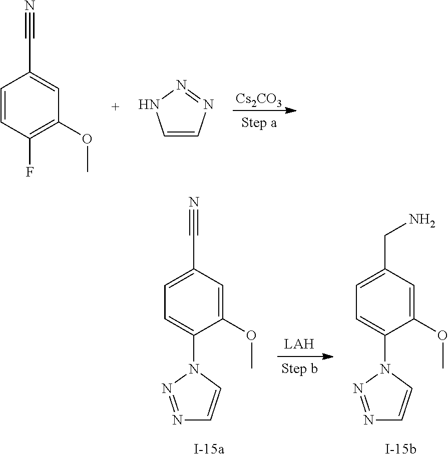

### Step a) 3-Methoxy-4-(1H-1,2,3-triazol-1-yl)benzonitrile (I-15a)

Cesium carbonate (80.7 g) and 1H-1,2,3-triazole (5.7 g) were added at rt to a stirred solution of 4-fluorobenzonitrile (10.0 g) in DMF (120.0 mL). The resulting reaction mixture was stirred under N₂ at 100° C. for 16 h, then diluted with water (500 mL), stirred for 15 min and filtered. The crude was combined with another batch (300 mg) and purified by column chromatography on silica gel eluted with a gradient of 30-40% EtOAc in pet ether, which gave the title compound (2.3 g, 23%) as a solid. LCMS (ES+) m/z 201.24 [M+H]⁺.

### Step b) (3-Methoxy-4-(1H-1,2,3-triazol-1-yl)phenyl)methanamine (I-15b)

Compound I-15a (2.0 g, 9.38 mmol) was reduced as described for intermediate 14 step b, which gave the title compound in 83% yield. LCMS (ES+) m/z 205.28 [M+H]⁺.

### Intermediate 16

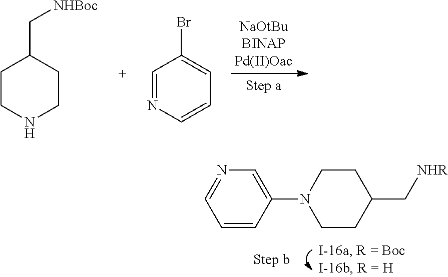

### Step a) tert-butyl ((1-(pyridin-3-yl)piperidin-4-yl)methyl)carbamate (I-16a)

NaOtBu (4.0 g, 42 mmol) and 3-bromopyridine (4.4 g, 28 mmol) were added to a stirred solution of tert-butyl piperidin-4-ylmethylcarbamate (3.0 g, 14 mmol) in dioxane in a sealed tube. The resulting mixture was purged with argon for 10 minutes followed by addition of BINAP (0.87 g, 1.4 mmol) and Pd(OAc)₂ (0.16 g, 0.7 mmol). The mixture was stirred at 100° C. for 16 h, then diluted with water and extracted with EtOAc. The combined organic layers were washed with brine, dried (Na₂SO₄), filtered and concentrated under reduced pressure. The afforded crude compound was purified by column chromatography on silica gel eluting with 3% MeOH in DCM, which gave the title compound (1.3 g, 22%). MS (ES+) m/z 292.29 [M+H]⁺.

### Step b) (1-(pyridin-3-yl)piperidin-4-yl)methanamine (I-16b)

4M HCl in dioxane (17 mL) was added at rt to a stirred solution of compound I-16a (2.5 g, 5.5 mmol) in dioxane. The solution was stirred for 3 h, then concentrated. The afford residue was washed with diethyl ether followed by pentane and dried under vacuum, which gave the HCl salt of title compound (1.5 g, 100%). MS (ES+) m/z 192.20 [M+H]⁺.

### Intermediate 17

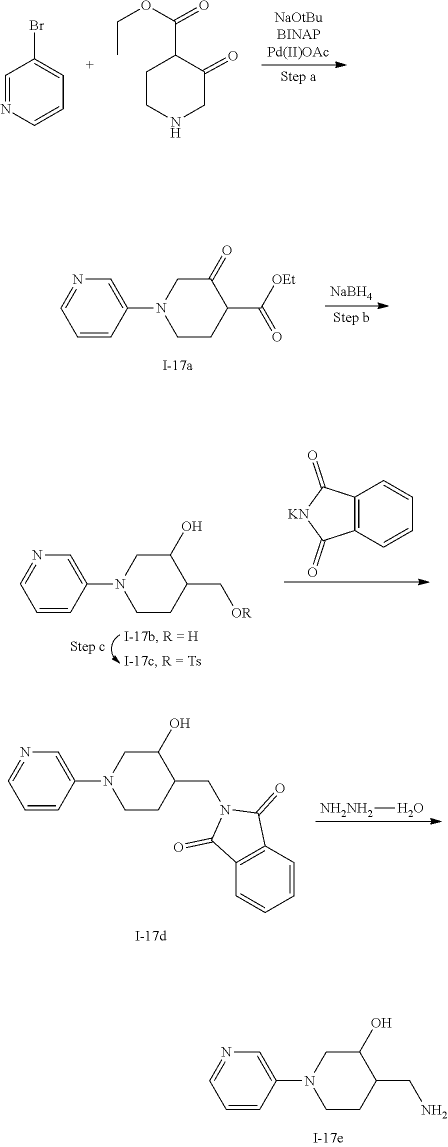

### Step a) ethyl 3-oxo-1-(pyridin-3-yl)piperidine-4-carboxylate (I-17a)

3-Bromopyridine (3.38 ml, 35.05 mmol), palladium acetate (0.2 g, 0.88 mmol) and BINAP (1.09 g, 1.75 mmol) were carefully added to a sealed tube charged with a degassed solution of ethyl 3-oxopiperidine-4-carboxylate (3.0 g, 17.5 mmol) and tBuONa (8.42 g, 87.6 mmol) in 1,4-dioxane (150 mL). The mixture was stirred at 80° C. for 16 h, then diluted with water and extracted with EtOAc (4×100 mL). The combined organic layers were washed with water and brine, dried (Na₂SO₄), filtered and concentrated under reduced pressure. The afforded crude compound was purified by column chromatography on silica gel eluting with 70% EtOAc in hexane, which gave the title compound (1.27 g, 27%). MS (ES+) m/z 249.18 [M+H]⁺.

### Step b) 4-(Hydroxymethyl)-1-(pyridin-3-yl)piperidin-3-ol (I-17b)

NaBH₄ (0.9 g, 23.84 mmol) was added at 0° C. to a solution of I-17a (2.13 g, 7.95 mmol) in EtOH (40 mL). The reaction mixture was stirred at rt. for 8 h, then water (1 mL) was added followed by 1N HCl to pH 7.5-8. The resulting mixture was concentrated and the afforded solid was triturated with 5% MeOH in DCM (300 mL). The solids were filtered off and the filtrate was concentrated. The crude product was purified by column chromatography on silica-gel eluted with a gradient of 5 to 10% MeOH in DCM, which gave the title compound (1.17 g, 71%) as a solid. LCMS (ES+) m/z 209.21 [M+H]⁺.

### Step c) (3-Hydroxy-1-(pyridin-3-yl)piperidin-4-yl)methyl 4-methylbenzenesulfonate (I-17c)

Tosyl chloride (769 mg, 4.03 mmol) was added at 0° C. to a solution of I-17b (421 mg, 2.02 mmol) in pyridine (10 mL). The resulting mixture was stirred at 0° C. for 3 h. Ice cold water was added and the reaction mixture was extracted with EtOAc (2×100 mL). The combined organic layers were washed with water (75 mL) and brine (75 mL), dried (Na₂SO₄) and concentrated under reduced pressure, which gave the title compound (675 mg) which was used in next step without further purification. (LCMS ES+) m/z 363.31 [M+H]⁺.

### Step d) 2-((3-Hydroxy-1-(pyridin-3-yl)piperidin-4-yl)methyl)isoindoline-1,3-dione (I-17d)

Potassium phthalimide (376 mg, 2.03 mmol) was added at rt to a solution of I-17c (675 mg, 1.35 mmol) in DMF (18 mL). The resulting mixture was stirred at 70° C. for 16 h, then ice cold water (20 mL) was added and the mixture was extracted with EtOAc (3×50 mL). The combined organic layers were washed with brine (50 mL), dried (Na₂SO₄), filtered and concentrated under reduced pressure. The obtained crude product was combined with additional batches and purified by silica-gel column chromatography eluted with a gradient of 5 to 10% MeOH in DCM which gave the title compound. LCMS (ES+) m/z 338.33 [M+H]⁺.

### Step e) 4-(Aminomethyl)-1-(pyridin-3-yl)piperidin-3-ol (I-17e)

Hydrazine hydrate (0.27 ml, 5.65 mmol) was added at rt to a solution of I-17d (681 mg, 1.61 mmol) in EtOH (15.0 mL). The resulting mixture was stirred at 70° C. for 16 h, then [0816] diluted with DCM (100 mL) and filtered through Celite. The filtrate was concentrated which gave the title compound (230 mg, 64%). LCMS (ES+) m/z 208.20 [M+H]⁺.

### Intermediate 18

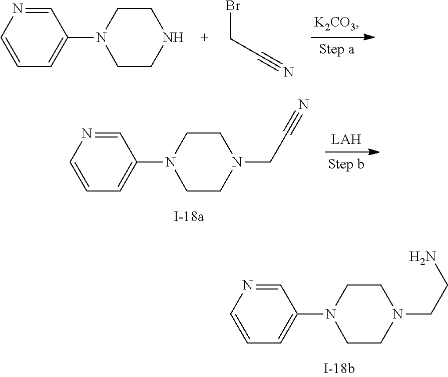

### Step a) 2-(4-(Pyridin-3-yl)piperazin-1-yl)acetonitrile (I-18a)

K₂CO₃ (931 mg, 6.74 mmol) and 2-bromoacetonitrile (0.23 mL, 3.37 mmol) were added at 0° C. to a stirred solution of 1-(pyridin-3-yl)piperazine (500 mg, 3.06 mmol) in acetonitrile (20 mL). The resulting reaction mixture was stirred at rt for 3 h, then filtered and concentrated under reduced pressure. The afforded crude compound (700 mg) was used in the next step without purification. LC-MS (ES+) m/z 203.16 [M+H]⁺.

### Step b) 2-(4-(Pyridin-3-yl)piperazin-1-yl)ethanamine (I-18b)

LiAlH₄ (263 mg, 0.01 mol) was added at 0° C. to a stirred solution of I-18a (700 mg, 3.46 mmol) in THE (10 mL). The resulting mixture was stirred at rt for 2 h, then the temperature was lowered to 0° C. and a sodium sulfate solution (8 mL) was added. The resulting mixture was stirred at rt for 1 h, then filtered through Celite and concentrated under reduced pressure. The obtained crude compound (440 mg) was used in the next step without purification. LC-MS (ES+) m/z 207.15 [M+H]⁺.

### Intermediate 19

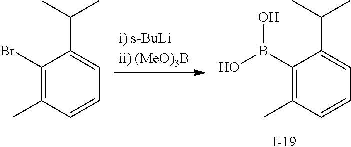

### (2-Isopropyl-6-methylphenyl)boronic acid (I-19)

1.4 M s-BuLi in pentane (4.51 mL) was added dropwise at −78° C. under argon to a solution of 2-bromo-1-isopropyl-3-methylbenzene (538 mg, 2.52 mmol) in dry THE (11 mL). The solution was stirred for 2 h at −78° C., then transferred through a cannula to a solution at -78° C. of triisopropyl borate (1.75 ml, 7.57 mmol) in dry THE (11 mL). The resulting mixture was stirred over night at rt, then 2M HCl (4 mL) was added and the mixture was extracted with ether (4×50 mL). The combined organic layers were washed with brine (75 mL), dried (Na₂SO₄), filtered and concentrated. The afforded crude was dissolved in EtOH (6.3 mL), NaOH (328 mg) was added and the mixture was stirred at rt for 30 min, then concentrated, acidified with 2M HCl and extracted with ether (4×50 mL). The combined organic layers were dried (Na₂SO₄), filtered and concentrated which gave the title compound the product (256 mg, 40%).

### Intermediate 20

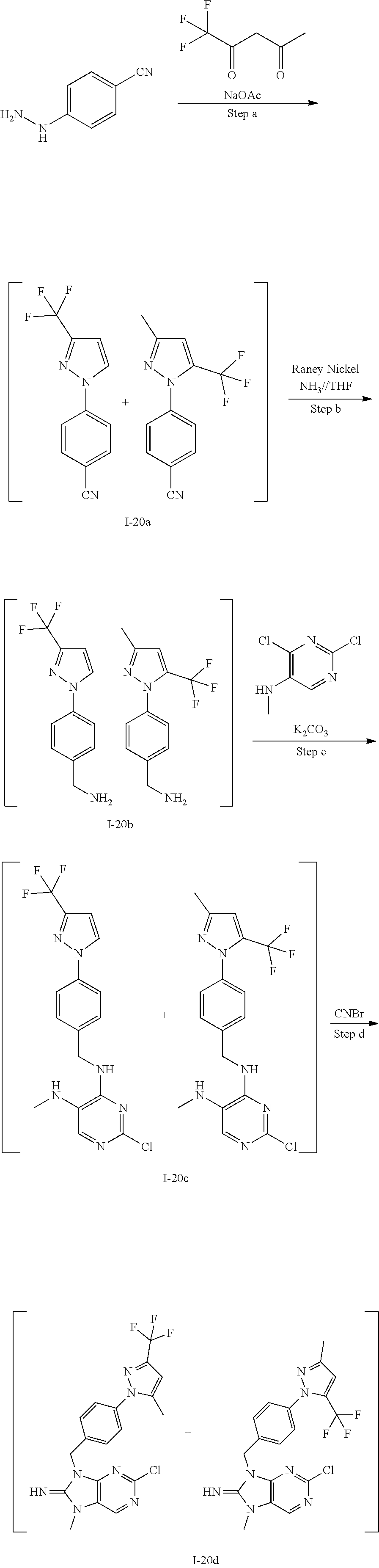

### Step a) 4-(3-methyl-5-(trifluoromethyl)-1H-pyrazol-1-yl)benzonitrile & 4-(5-methyl-3-(trifluoromethyl)-1H-pyrazol-1-yl)benzonitrile (I-20a)

Sodium acetate (6.2 g, 75 mmol) was added to a stirred solution of 4-hydrazinylbenzonitrile (5 g, 38 mmol) and 1,1,1-trifluoropentane-2,4-dione (7.2 g, 47 mmol) in acetic acid (25 mL) and the resulting mixture was heated at 120° C. for 1 h, then concentrated under reduced pressure. The crude product was purified by column chromatography on silica-gel eluted with a gradient of 10-35% EtOAc in pet ether, which gave the mixture of title compounds (6.0 g, 31%) as a solid. LCMS (ES+) m/z 252.18 [M+H]⁺.

### Step b) (4-(3-methyl-5-(trifluoromethyl)-1H-pyrazol-1-yl)phenyl)methanamine & (4-(5-methyl-3-(trifluoromethyl)-1H-pyrazol-1-yl)phenyl)methanamine (I-20b)

Compound I-20a (2 g, 4.0 mmol) was added to a solution of Raney nickel (250 mg, washed with acetone) in EtOH (50 mL) followed by addition of 7M NH₃ in THF (3 mL). The reaction mixture was stirred under hydrogen balloon at rt for 12 h, then the reaction mixture was filtered through Celite bed, washed with 10% MeOH in DCM and the filtrate was concentrated under reduced pressure, which gave the mixture of title compounds (2 g, 41%) as a liquid. LCMS (ES+) m/z 255.22 [M+H]⁺. The compound was taken to next step without further purification.

### Step c) 2-chloro-N5-methyl-N4-(4-(3-methyl-5-(trifluoromethyl)-1H-pyrazol-1-yl)benzyl)pyrimidine-4,5-diamine & 2-chloro-N5-methyl-N4-(4-(5-methyl-3-(trifluoromethyl)-1H-pyrazol-1-yl)benzyl)pyrimidine-4,5-diamine (I-20c)

K₂CO₃ (3.1 g, 22 mmol) and compound I-20b (3.1 g, 6.2 mmol) were added at 0° C. to a stirred solution of 2,4-dichloro-N-methylpyrimidin-5-amine (1 g, 5.6 mmol) in DMF (25 mL). The resulting reaction mixture was stirred at 90° C. for 12 h, then dissolved in water and extracted with EtOAc. The combined organic layer was washed with brine, dried (Na₂SO₄), filtered and concentrated under reduced pressure. The crude product was purified by column chromatography on silica-gel eluted with a gradient of 30-65% EtOAc in pet ether, which gave the mixture of title compounds (1.0 g, 12%) as a solid. LCMS (ES+) m/z 397.31 [M+H]⁺.

### Step d) 2-chloro-7-methyl-9-(4-(3-methyl-5-(trifluoromethyl)-1H-pyrazol-1-yl)benzyl)-7,9-dihydro-8H-purin-8-imine & 2-chloro-7-methyl-9-(4-(5-methyl-3-(trifluoromethyl)-1H-pyrazol-1-yl)benzyl)-7,9-dihydro-8H-purin-8-imine (I-20d)

Cyanogen bromide (480 mg, 4.5 mmol) was added at rt to a stirred solution of compound I-20c (1 g, 1.13 mmol) in EtOH (25 mL). The resulting mixture was stirred at 80° C. for 16 h, then concentrated under reduced pressure. The afforded residue was purified by column chromatography on silica gel, eluted with 3-6% MeOH in DCM, which gave the title compound (450 mg, 13%) as a solid. LCMS (ES+) 422.28 [M+H]⁺.

### Intermediate 21

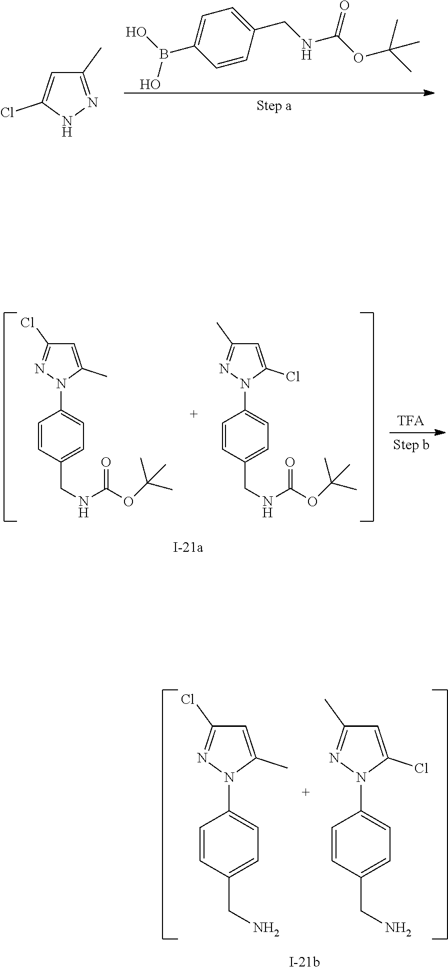

### Step a) tert-butyl (4-(3-chloro-5-methyl-1H-pyrazol-1-yl)benzyl)carbamate & tert-butyl (4-(5-chloro-3-methyl-1H-pyrazol-1-yl)benzyl)carbamate (I-21a)

(4-(((tert-butoxycarbonyl)amino)methyl)phenyl)boronic acid (2.6 g, 10 mmol), copper(II) acetate (2 g, 11.2 mmol), Et₃N (2.5 mL, 18 mmol) and pyridine (2.3 mL, 28.3 mmol) were added at rt to a stirred solution of 5-chloro-3-methyl-1H-pyrazole (1.0 g, 8.6 mmol) in DCM (100 mL). The resulting reaction mixture was stirred at 40° C. for 16 h, then filtered through the celite bed. The filtrate was concentrated under reduced pressure. The crude compound was purified by column chromatography on silica gel and eluted with 30% EtOAc in pet ether, which gave the inseparable mixture of title compounds (1.1 g, 15%) as a liquid. LCMS (ES+) m/z: 322.24 [M+H]⁺.

### Step b) (4-(3-chloro-5-methyl-1H-pyrazol-1-yl)phenyl)methanamine & (4-(5-chloro-3-methyl-1H-pyrazol-1-yl)phenyl)methanamine (I-21b)

To a stirred solution of compound I-21a (1.1 g, 1.7 mmol) in DCM (20 mL) was added TFA (2 g, 17.1 mmol). The resulting reaction mixture was stirred at rt for 16 h, then concentrated under reduced pressure, which gave the inseparable mixture of title compounds (750 mg, 93%) as a liquid. LCMS (ES+) m/z: 222.22 [M+H]⁺.

### Intermediate 22

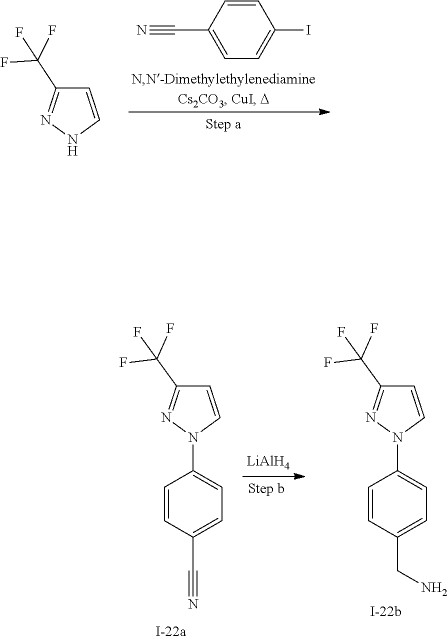

### Step a) 4-(3-(trifluoromethyl)-1H-pyrazol-1-yl)benzonitrile (I-22a)

Copper iodide (83 mg, 0.44 mmol) and N,N′-dimethylethylenediamine (77 mg, 0.9 mmol) were added to a stirred solution of 4-iodobenzonitrile (1.0 g, 4.4 mmol), 3-(trifluoromethyl)-1H-pyrazole (900 mg, 6.6 mmol) and K₂CO₃ (1.2 g, 8.7 mmol) in 1,4-dioxane (10 mL). The mixture was stirred under argon at 100° C. for 2 h in a microwave, then reaction mixture was filtered through the celite bed. The filtrate was diluted with water (50 mL) and the mixture was extracted with EtOAc (3×50 mL). The combined organic layer was washed with brine (100 mL), dried (Na₂SO₄), filtered and concentrated. The crude compound was purified by column chromatography on neutral alumina and eluted with 10% EtOAc in pet ether, which gave the inseparable title compound (900 mg, 86%) as a solid. LCMS (ES+) m/z: 238.16 [M+H]⁺.

### Step b) (4-(3-(trifluoromethyl)-1H-pyrazol-1-yl)phenyl)methanamine (I-22b)

LiAlH₄ (solid) (256 mg, 6.7 mmol) was added at 0° C. to a stirred solution of compound I-22a (800 mg, 3.4 mmol) in dry THE (50 mL). The resulting reaction mixture was stirred at rt until TLC indicated complete consumption of starting material (2 h), then the temperature was lowered to 0° C. and sodium sulfate solution (15 mL) was added. The resulting mixture was stirred at rt for 30 min, then filtered through Celite bed and the filtrate was concentrated under reduced pressure, which gave the title compound (800 mg, 93%) as a liquid. MS (ES+) 242.18 [M+H]⁺.

### Intermediate 23

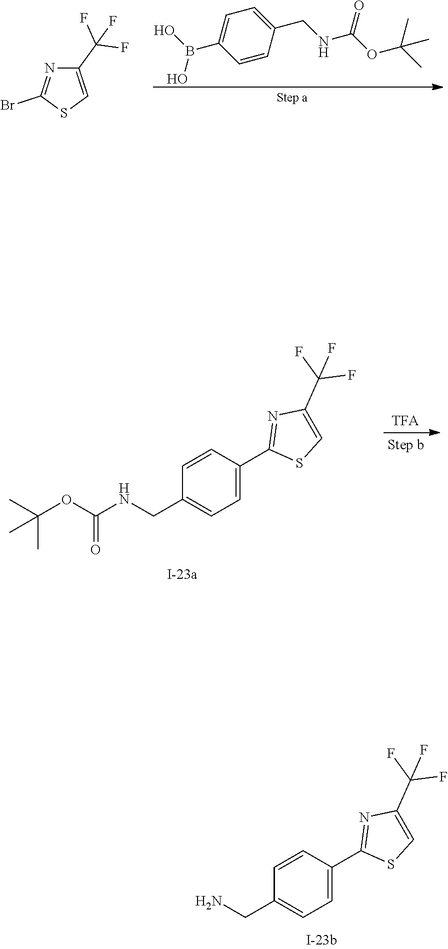 [SMILES: FC(F)(F)c1csc(Br)n1 ; CC(C)(C)OC(=O)NCc1ccc(B(O)O)cc1 ; CC(C)(C)OC(=O)NCc1ccc(-c2nc(C(F)(F)F)cs2)cc1 ; "TFA" ; NCc1ccc(-c2nc(C(F)(F)F)cs2)cc1]

### Step a) tert-butyl (4-(4-(trifluoromethyl)thiazol-2-yl)benzyl)carbamate (I-23a)

(4-(((tert-butoxycarbonyl)amino)methyl)phenyl)boronic acid (1.3 g, 5.2 mmol)) was added to a stirred solution of 2-bromo-4-(trifluoromethyl)thiazole (1 g, 4.3 mmol) and sodium carbonate (1.8 g, 17.2 mmol) in toluene (15 mL), EtOH (15 mL) and water (3 mL). The reaction mixture was degassed by bubbling with argon for 10 minutes then Pd(PPh₃)₄ (500 mg, 0.43 mmol) was added and the reaction mixture was degassed for 2 min. The resulting reaction mixture was stirred at 120° C. for 16 h in sealed tube, then filtered through the celite bed, the filtrate was concentrated under reduced pressure. The afforded crude compound was purified by column chromatography on silica gel, eluted with 20-30% EtOAc/hexane, which gave the title compound (1.1 g, 69%) as a solid. LCMS (ES+) m/z 359.27 [M+H]⁺.

### Step b) (4-(4-(trifluoromethyl)thiazol-2-yl)phenyl)methanamine (I-23b)

To a stirred solution of compound I-23a (1.0 g, 2.8 mmol) in DCM (10 mL) was added TFA (1.6 g, 14 mmol). The resulting reaction mixture was stirred at rt for 3 h, then concentrated under reduced pressure. The residue was triturated with pentane and dried, which gave title compound (700 mg, 95%) as a solid. LCMS (ES+) m/z: 259.17 [M+H]⁺.

### Intermediate 24

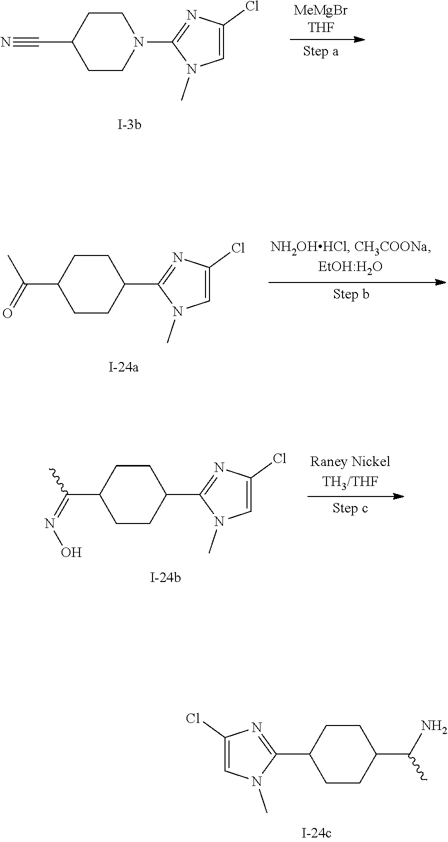

### Step a) 1-(4-(4-chloro-1-methyl-1H-imidazol-2-yl)cyclohexyl)ethan-1-one (I-24a)

MeMgBr (1M in THF) (37.3 mL, 37.3 mmol) was added at 0° C. to a stirred solution of compound I-3b (3.0 g, 12.4 mmol) in THF (60 mL). The resulting mixture was stirred at 70° C. for 8 h, then the reaction was quenched with ammonium chloride solution at 10° C. and extracted with EtOAc. The combined organic layer was dried (Na₂SO₄), filtered and concentrated under reduced pressure. The crude compound was purified by column chromatography on silica gel and eluted with 40-50% EtOAc in pet ether, which gave the title compound (2.5 g, 69%) as a solid. MS (ES+) 242.15 [M+H]⁺.

### Step b) 1-(4-(4-chloro-1-methyl-1H-imidazol-2-yl)cyclohexyl)ethan-1-one oxime (I-24b)

Sodium acetate (2.4 g, 29.1 mmol) was added to a stirred solution of compound I-24a (4 g, 14.6 mmol) in EtOH (40 mL) and water (10 mL), followed by addition of hydroxylamine hydrochloride (2.0 g, 29.1 mmol). The resulting mixture was heated at 85° C. for 4 h, then concentrated under reduced pressure. Water was added and extracted with EtOAc. The combined organic layer was dried (Na₂SO₄), filtered and concentrated under reduced pressure, which gave the crude title compound (3.5 g, 62%) as a solid. LCMS (ES+) m/z 257.19 [M+H]⁺.

### Step c) 1-(4-(4-chloro-1-methyl-1H-imidazol-2-yl)cyclohexyl)ethan-1-amine (I-24c)

Raney nickel (1.8 g, 31.3 mmol) was added to a solution of compound I-24b (3.0 g, 7.8 mmol) in MeOH (50 mL), then 7M NH₃ in MeOH (5.0 mL) was added. The reaction mixture was stirred in a Parr shaker at 60 psi at 50° C. for 16 h, then the reaction mixture was filtered through Celite bed, washed with MeOH (30 mL) and the filtrate was concentrated under reduced pressure. The crude compound was purified by column chromatography on neutral alumina and eluted with 2% MeOH in DCM, which gave the title compound (600 mg, 26%) as a semi solid. MS (ES+) 243.23 [M+H]⁺.

### Intermediate 25

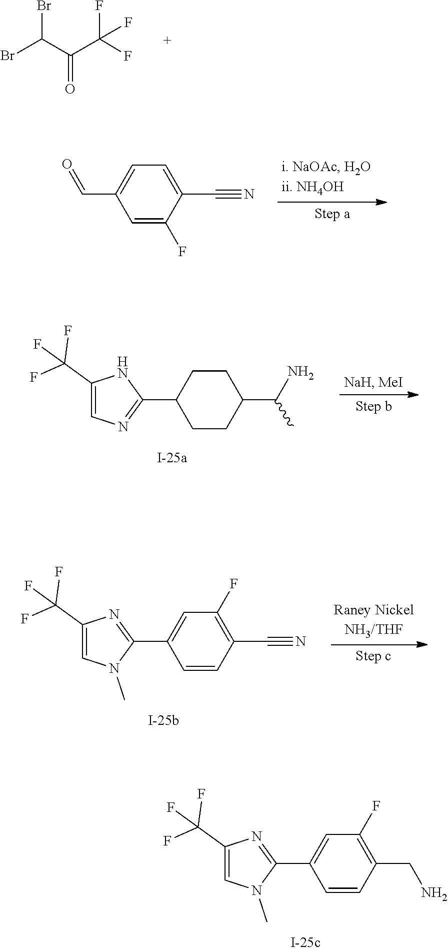 [SMILES: O=C(C(Br)Br)C(F)(F)F ; N#Cc1ccc(C=O)cc1F ; CC(N)C1CCC(c2ncc(C(F)(F)F)[nH]2)CC1 ; Cn1cc(C(F)(F)F)nc1-c1ccc(C#N)c(F)c1 ; Cn1cc(C(F)(F)F)nc1-c1ccc(CN)c(F)c1]

### Step a) 2-fluoro-4-(5-(trifluoromethyl)-1H-imidazol-2-yl)benzonitrile (I-25a)

A mixture of 3,3-dibromo-1,1,1-trifluoropropan-2-one (9 g, 33.4 mmol) and sodium acetate (2.7 g, 33.4 mmol) in water (20 mL) was heated at 100° C. for 45 min, then was cooled to rt. The mixture was added to a solution of 2-fluoro-4-formylbenzonitrile (5.0 g, 33.4 mmol) in MeOH (100 mL) followed by addition of 35% aq. NH₄OH (30 g, 299.3 mmol). The resulting reaction mixture was stirred at rt for 45 min, heated at 100° C. for 1 h, then concentrated under reduced pressure. Water (60 mL) was added to the residue and stirred for 20 min. The precipitated solid was filtered and dried. The crude compound was purified by column chromatography on silica gel and eluted with 20% EtOAc in pet ether, which gave the title compound (3.5 g, 35%) as a solid. LCMS (ES+) m/z 256.21 [M+H]⁺.

### Step b) 2-fluoro-4-(1-methyl-4-(trifluoromethyl)-1H-imidazol-2-yl)benzonitrile (I-25b)

NaH (60%, 1.0 g, 25.5 mmol) was added at 0° C. to a solution of compound I-25a (3.5 g, 12.8 mmol) in THF (50 ml), then CH₃I (1.2 mL, 19.1 mmol) was added at 0° C. and stirring was then continued for 16 h at rt. Ice water was added and the mixture was extracted with EtOAc (1×100 mL). The combined organic layer was washed with brine (100 mL), dried (Na₂SO₄), filtered and concentrated. The crude compound was purified twice by column chromatography on silica gel and eluted with 20% acetone in pet ether, which gave the title compound (1.9 g, 51%) as a solid. MS (ES+) 270.19 [M+H]⁺.

### Step c) (2-fluoro-4-(1-methyl-4-(trifluoromethyl)-1H-imidazol-2-yl)phenyl)methanamine (I-25c)

Compound I-25b (100 mg, 0.4 mmol) and 0.4M NH₃ in THF (0.63 mL, 0.3 mmol) was added to a suspension of Raney nickel (63 mg, 1.1 mmol) in EtOH (10 mL). The reaction mixture was stirred under hydrogen balloon at rt for 3 h, then the reaction mixture was filtered through Celite bed, washed with EtOAc and the filtrate was concentrated under reduced pressure, which gave the title compound (100 mg, 28%). MS (ES+) 273.23 [M+H]⁺.

### Intermediate 26

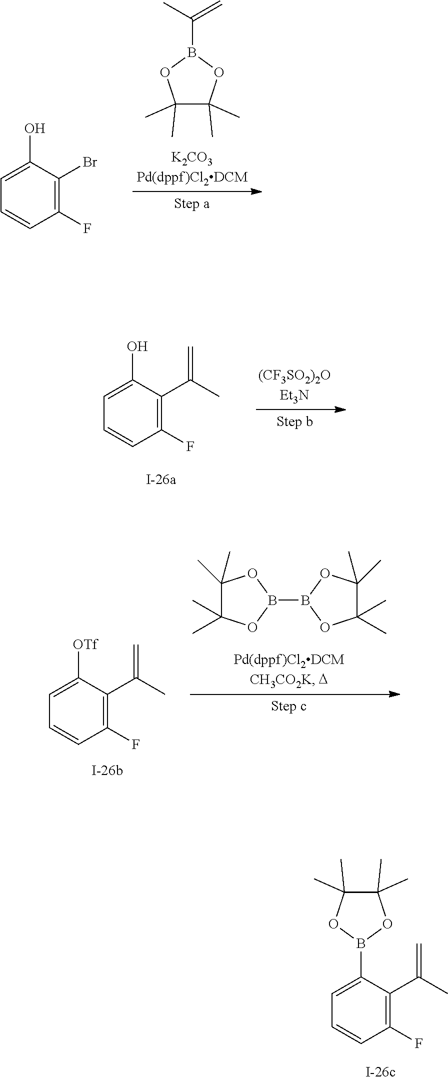

### Step a) 3-fluoro-2-(prop-1-en-2-yl)phenol (I-26a)

To a stirred solution of 2-bromo-3-fluorophenol (10 g, 52.4 mmol) and 4,4,5,5-tetramethyl-2-(prop-1-en-2-yl)-1,3,2-dioxaborolane (9.7 g, 57.6 mmol) in 1,4-dioxane (150 mL) and water (10 mL). The reaction mixture was degassed by bubbling with argon for 10 min, K₂CO₃ (14.5 g, 105 mmol) was added followed by addition of Pd(dppf)Cl₂·DCM (4.3 g, 5.2 mmol). and the mixture was stirred at 70° C. for 16 h. The reaction mixture was diluted with water, filtered through the celite bed. The filtrate was extracted with EtOAc. The combined organic layer was washed with brine, dried (Na₂SO₄), filtered and concentrated under reduced pressure. The afforded crude compound was purified by column chromatography on silica gel, eluted with 5% EtOAc in pet ether, which gave the title compound (5 g, 54%) as a solid. LCMS (ES−) m/z 151.21 [M−H]⁻.

### Step b) 3-fluoro-2-(prop-1-en-2-yl)phenyl trifluoromethanesulfonate (I-26b)

Et₃N (5.5 mL, 39.4 mmol) was added at 0° C. to a solution of compound I-26a (3 g, 19.7 mmol) in DCM (25 mL), then triflic anhydride (3.6 mL, 21.7 mmol) was added at 0° C. The reaction mixture was stirred for 3 h at same temperature, then diluted with DCM. The organic layer was washed with water, brine, dried (Na₂SO₄) and concentrated under reduced pressure. The crude product was purified by column chromatography on silica gel eluted with pet ether, which gave the title compound (3 g, 50%) as a liquid.

### Step c) 2-(3-fluoro-2-(prop-1-en-2-yl)phenyl)-4,4,5,5-tetramethyl-1,3,2-dioxaborolane (I-26c)

Potassium acetate (660 mg, 6.7 mmol) and 4,4,4′,4′,5,5,5′,5′-octamethyl-2,2′-bi(1,3,2-dioxaborolane) (1.7 g, 6.7 mmol) were added to a solution of compound I-26b (1 g, 3.3 mmol) in 1,4-dioxane (20 mL). The resulting mixture was de-gassed for 15 min with argon, then bis(triphenylphosphine)palladium(II) dichloride (275 mg, 0.33 mmol) was added, the mixture was de-gassed for 5 min, then stirred at 80° C. for 16 h in a sealed tube. The reaction mixture was filtered through the celite bed. The filtrate was concentrated under reduced pressure. The afforded crude compound was purified by column chromatography on silica gel, eluted with 5-10% EtOAc in pet ether, which gave the title compound (350 mg, 19%) as a semi-solid.

### Intermediate 27

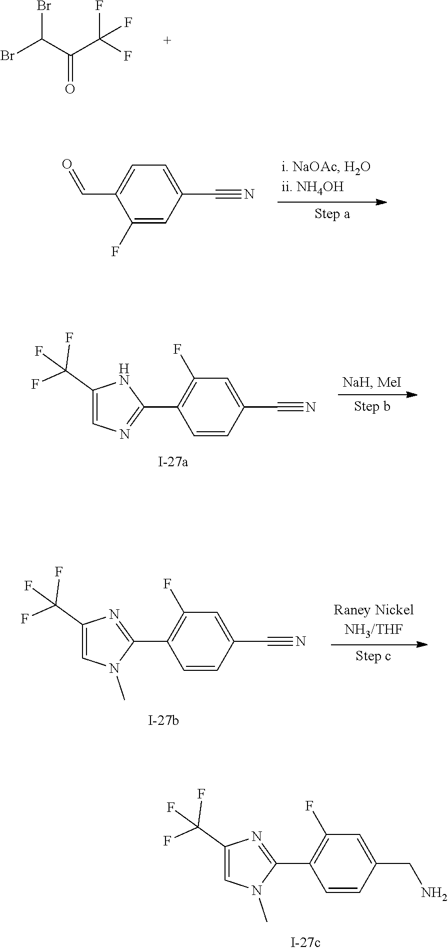 [SMILES: O=C(C(Br)Br)C(F)(F)F ; N#Cc1ccc(C=O)c(F)c1 ; N#Cc1ccc(-c2ncc(C(F)(F)F)[nH]2)c(F)c1 ; Cn1cc(C(F)(F)F)nc1-c1ccc(C#N)cc1F ; Cn1cc(C(F)(F)F)nc1-c1ccc(CN)cc1F]

### Step a) 3-fluoro-4-(5-(trifluoromethyl)-1H-imidazol-2-yl)benzonitrile (I-27a)

A mixture of 3,3-dibromo-1,1,1-trifluoropropan-2-one (18 g, 66.7 mmol) and sodium acetate (5.5 g, 66.7 mmol) in water (10 mL) was heated at 100° C. for 45 min, then was cooled to rt. The mixture was added to a solution of 3-fluoro-4-formylbenzonitrile (9.95 g, 66.7 mmol) in MeOH (150 mL) followed by addition of 35% aq. NH₄OH (60 g, 598.6 mmol). The resulting reaction mixture was stirred at rt for 45 min, heated at 100° C. for 1 h, then concentrated under reduced pressure. Water (60 mL) was added to the residue and stirred for 20 min. The precipitated solid was filtered and dried. The crude compound was triturated with diethyl ether, which gave the title compound (10 g, 52%) as a solid. LCMS (ES+) m/z 256.12 [M+H]⁺.

### Step b) 2-fluoro-4-(1-methyl-4-(trifluoromethyl)-1H-imidazol-2-yl)benzonitrile (I-27b)

NaH (60%, 2.9 g, 72.9 mmol) was added at 0° C. to a solution of compound I-27a (10 g, 36.5 mmol) in THF (100 mL), then CH₃I (3.4 mL, 54.7 mmol) was added at 0° C. and the stirring was continued for 16 h at rt. Ice cold water (50 mL) was added and the mixture was extracted with EtOAc (3×100 mL). The combined organic layers were washed with brine (100 mL), dried (Na₂SO₄), filtered and concentrated. The crude compound was purified by column chromatography on silica gel and eluted with 20% acetone in pet ether, which gave the title compound (4.5 g, 38%) as a solid. MS (ES+) 270.19 [M+H]⁺.

### Step c) (2-fluoro-4-(1-methyl-4-(trifluoromethyl)-1H-imidazol-2-yl)phenyl)methanamine (I-27c)

Compound I-27b (400 mg, 1.5 mmol) was added to a suspension of Raney nickel (250 mg, 4.3 mmol) and 0.4M NH₃ in THF (2.5 mL, 1.0 mmol) in EtOH (20 mL). The reaction mixture was stirred under hydrogen balloon at rt for 3 h, then the reaction mixture was filtered through Celite bed, washed with EtOAc and the filtrate was concentrated under reduced pressure, which gave the title compound (380 mg, 51%). MS (ES+) 273.21 [M+H]⁺.

### Intermediate 28

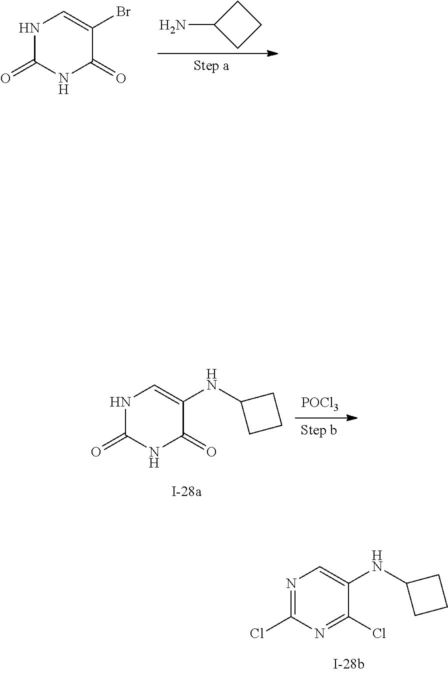

### Step a) 5-(cyclobutylamino)pyrimidine-2,4(1H,3H)-dione (I-28a)

A mixture of 5-bromopyrimidine-2,4(1H,3H)-dione (20 g, 104.7 mmol) and cyclobutanamine (10.7 mL, 156.11 mmol) was heated at 90° C. for 1 h. Water was added to the residue and the precipitated solid was filtered and dried, which gave the title compound (18.2 g, 77%) as a solid. LCMS (ES+) 182.31 [M+H]⁺.

### Step b) 2,4-dichloro-N-cyclobutylpyrimidin-5-amine (I-28b)

To a stirred suspension of compound I-28a (15 g, 67.1 mmol) in phosphorus oxychloride (31.5 mL, 336.9 mmol) was added Et₃N (18.6 mL, 133.4 mmol) dropwise at 0° C. The mixture was refluxed for 16 h at 100° C., then cooled to rt and concentrated under reduced pressure. Ice water was added to the residue and basified with saturated NaHCO₃ solution. The aqueous layer was extracted with EtOAc (twice). The combined organic layer was dried (Na₂SO₄), filtered and concentrated. The crude product was purified by column chromatography on silica gel eluted with 20-30% EtOAc in pet ether which gave the title compound (5.2 g, 22%) as a solid. LCMS (ES+) m/z 218.26 [M+H]⁺.

### Intermediate 29

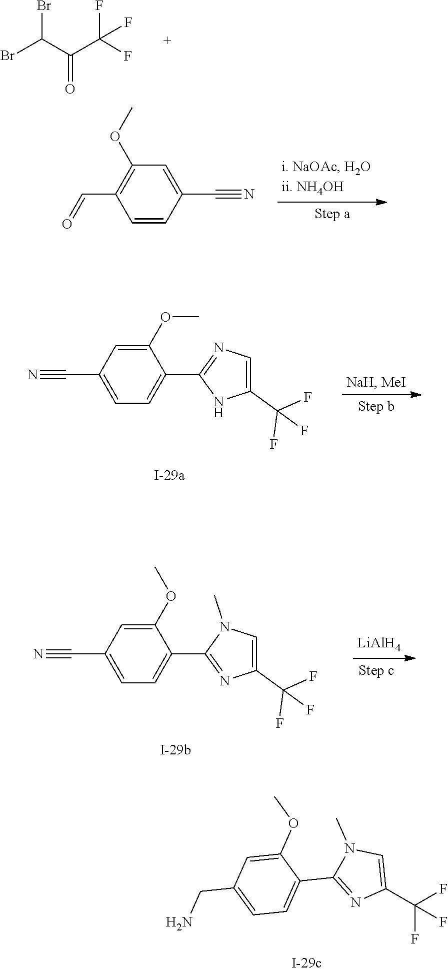 [SMILES: O=C(C(Br)Br)C(F)(F)F ; COc1cc(C#N)ccc1C=O ; COc1cc(C#N)ccc1-c1ncc(C(F)(F)F)[nH]1 ; COc1cc(C#N)ccc1-c1nc(C(F)(F)F)cn1C ; "LiAlH4" ; COc1cc(CN)ccc1-c1nc(C(F)(F)F)cn1C]

### Step a) 3-methoxy-4-(5-(trifluoromethyl)-1H-imidazol-2-yl)benzonitrile (I-29a)

A mixture of 3,3-dibromo-1,1,1-trifluoropropan-2-one (10 g, 36.7 mmol) and sodium acetate (3 g, 36.6 mmol) in water (50 mL) was heated at 100° C. for 1 h, then was cooled to rt. The mixture was added to a solution of 4-formyl-3-methoxybenzonitrile (5.9 g, 36.6 mmol) in MeOH (100 mL) followed by addition of 35% aq. NH₄OH (37 g, 369.5 mmol). The resulting reaction mixture was stirred at rt for 1 h, heated at 100° C. for 1 h, then concentrated under reduced pressure. Water (60 mL) was added to the residue and stirred for 20 min. The precipitated solid was filtered and dried, which gave the title compound (9.5 g, 87%) as a solid. LCMS (ES+) m/z 268.15 [M+H]⁺.

### Step b) 3-methoxy-4-(1-methyl-4-(trifluoromethyl)-1H-imidazol-2-yl)benzonitrile (I-29b)

NaH (60%, 1.3 g, 32.5 mmol) was added portionwise at 0° C. to a solution of compound I-29a (9.5 g, 32 mmol) in THE (100 mL). The mixture was stirred for 30 min at 0° C., then CH₃I (2 mL, 32.1 mmol) was added and stirred for 4 h at rt. The reaction was quenched by adding ice water and the mixture was extracted with EtOAc (twice). The combined organic layers were washed with brine, dried (Na₂SO₄), filtered and concentrated under reduced pressure. The crude compound was purified by column chromatography on silica gel and eluted with 15-20% EtOAc in pet ether, which gave the title compound (3.8 g, 41%) as a solid. MS (ES+) 282.23 [M+H]⁺.

### Step c) (3-methoxy-4-(1-methyl-4-(trifluoromethyl)-1H-imidazol-2-yl)phenyl)methanamine (I-29c)

To a suspension of LiAlH₄ (solid) (470 mg, 12.4 mmol) in dry THE (80 mL) was added a solution of compound I-29b (2 g, 6.0 mmol) in THE (20 mL) dropwise at 0° C. The resulting reaction mixture was stirred at rt until TLC indicated complete consumption of starting material (2 h), then the temperature was lowered to 0° C. and sodium sulfate solution was added. The resulting mixture was stirred at rt for 10 min, then filtered through Celite bed and washed with EtOAc. The filtrate was concentrated under reduced pressure. The obtained residue was dissolved in EtOAc dried (Na₂SO₄), filtered and concentrated under reduced pressure, which gave the title compound (1.8 g, 97%) as a liquid. MS (ES+) 286.27 [M+H]⁺.

### Intermediate 30

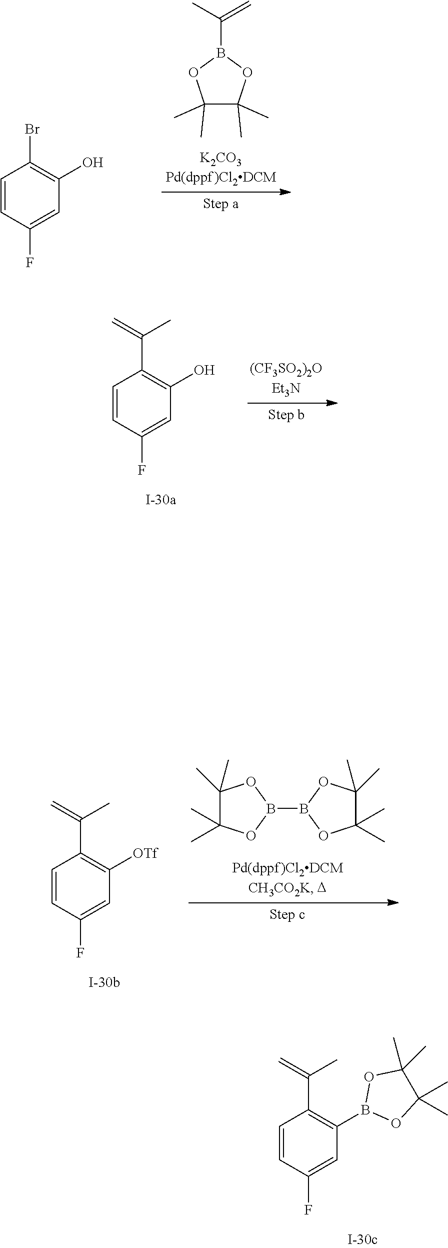

### Step a) 5-fluoro-2-(prop-1-en-2-yl)phenol (I-30a)

A stirred solution of 2-bromo-5-fluorophenol (4 g, 21 mmol) and 4,4,5,5-tetramethyl-2-(prop-1-en-2-yl)-1,3,2-dioxaborolane (4.2 g, 25.1 mmol) in 1,4-dioxane (40 mL) and water (5 mL) was degassed by bubbling with argon for 10 min, then K₂CO₃ (5.3 g, 38 mmol) was added followed by addition of Pd(dppf)Cl₂·DCM (1.6 g, 1.9 mmol) and the mixture was stirred at 70° C. for 16 h. The reaction mixture was diluted with water, filtered through the celite bed. The filtrate was extracted with EtOAc. The combined organic layer was washed with brine, dried (Na₂SO₄), filtered and concentrated under reduced pressure. The afforded crude compound was purified by column chromatography on silica gel, eluted with 5% EtOAc in pet ether, which gave the title compound (2 g, 41%) as a solid. LCMS (ES−) m/z 151.29 [M−H]⁻.

### Step b) 5-fluoro-2-(prop-1-en-2-yl)phenyl trifluoromethanesulfonate (I-30b)

Et₃N (1.8 mL, 13 mmol) was added at 0° C. to a solution of compound I-30a (2 g, 8.7 mmol) in DCM (15 mL), then triflic anhydride (1.6 mL, 9.5 mmol) was added at 0° C. The reaction mixture was stirred for 3 h at same temperature, then diluted with DCM. The organic layer was washed with water, brine, dried (Na₂SO₄) and concentrated under reduced pressure. The crude product was purified by column chromatography on silica gel eluted with pet ether, which gave the title compound (1 g, 36%) as a liquid.

### Step c) 2-(5-fluoro-2-(prop-1-en-2-yl)phenyl)-4,4,5,5-tetramethyl-1,3,2-dioxaborolane (I-30c)

4,4,4′,4′,5,5,5′,5′-Octamethyl-2,2′-bi(1,3,2-dioxaborolane) (1.6 g, 6.3 mmol) and potassium acetate (622 mg, 6.3 mmol) were added to a solution of compound I-30b (1 g, 3.2 mmol) in 1,4-dioxane (15 mL). The resulting mixture was de-gassed for 15 min with argon, then bis(triphenylphosphine)palladium(II) dichloride (260 mg, 0.32 mmol) was added, the mixture was de-gassed for 5 min, then stirred at 80° C. for 16 h. The reaction mixture was filtered through the celite bed. The filtrate was concentrated under reduced pressure. The afforded crude compound was purified by column chromatography on silica gel, eluted with 10% EtOAc in pet ether, which gave the title compound (800 mg, 38%) as a semi-solid. LCMS (ES−) m/z 263.25 [M+H]⁺.

### Intermediate 31

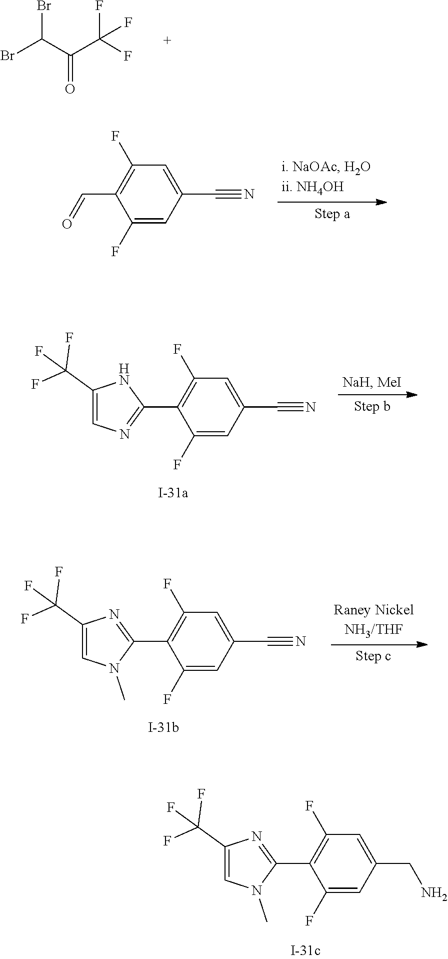 [SMILES: O=C(C(Br)Br)C(F)(F)F ; N#Cc1cc(F)c(C=O)c(F)c1 ; N#Cc1cc(F)c(-c2ncc(C(F)(F)F)[nH]2)c(F)c1 ; Cn1cc(C(F)(F)F)nc1-c1c(F)cc(C#N)cc1F ; Cn1cc(C(F)(F)F)nc1-c1c(F)cc(CN)cc1F]

### Step a) 3,5-difluoro-4-(5-(trifluoromethyl)-1H-imidazol-2-yl)benzonitrile (I-31a)

A mixture of 3,3-dibromo-1,1,1-trifluoropropan-2-one (8 g, 29.7 mmol) and sodium acetate (2.9 g, 35.6 mmol) in water (10 mL) was heated at 100° C. for 45 min, then was cooled to rt. The mixture was added to a solution of 3,5-difluoro-4-formylbenzonitrile (5 g, 29.7 mmol) followed by addition of 35% aq. NH₄OH (26.6 g, 266.1 mmol) in MeOH (100 mL). The resulting reaction mixture was stirred at rt for 45 min, heated at 100° C. for 1 h, then concentrated under reduced pressure. Water (60 mL) was added to the residue and stirred for 20 min. The precipitated solid was filtered and dried. The crude compound was triturated with diethyl ether/n-pentane. The residue was further purified by column chromatography on silica gel and eluted with 20% EtOAc in pet ether, which gave the title compound (3.5 g, 40%) as a solid. LCMS (ES+) m/z 274.30 [M+H]⁺.

### Step b) 3,5-difluoro-4-(1-methyl-4-(trifluoromethyl)-1H-imidazol-2-yl)benzonitrile (I-31b)

NaH (60%, 550 mg, 13.6 mmol) was added at 0° C. to a solution of compound I-31a (2 g, 6.8 mmol) in THF (50 mL), then CH₃I (0.7 mL, 10.2 mmol) was added at 0° C. and the stirring was continued for 16 h at rt. Ice water was added and the mixture was extracted with EtOAc (100 mL). The combined organic layers were washed with brine (100 mL), dried (Na₂SO₄), filtered and concentrated under reduced pressure. The crude compound was purified by column chromatography on silica gel and eluted with 5% acetone in pet ether, which gave the title compound (1.2 g, 49%) as a solid. MS (ES+) 288.28 [M+H]⁺.

### Step c) (3,5-difluoro-4-(1-methyl-4-(trifluoromethyl)-1H-imidazol-2-yl)phenyl)methanamine (I-31c)

Compound I-31b (1 g, 3.5 mmol) was added to a suspension of Raney nickel (590 mg, 10.0 mmol) in EtOH (20 mL), then 0.4M NH₃ in THE (5.9 mL, 2.3 mmol) The reaction mixture was stirred under hydrogen balloon at rt for 3 h, then the reaction mixture was filtered through Celite bed, washed with EtOAc and the filtrate was concentrated under reduced pressure, which gave the title compound (832 mg, 41%). MS (ES+) 292.33 [M+H]⁺.

### Intermediate 32

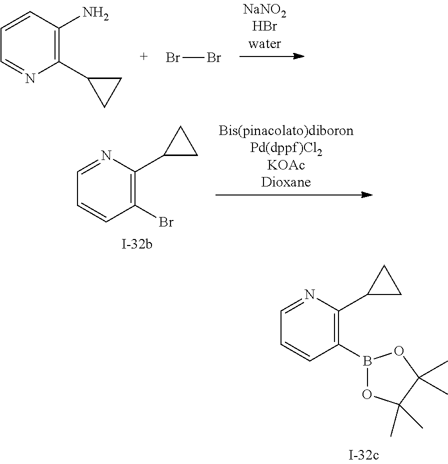

### Step a) 3-bromo-2-cyclopropyl-pyridine

2-cyclopropylpyridin-3-amine (5.00 g, 37.3 mmol) was suspended in Hydrobromic acid, 48% wt in water, (16.2 mL). The reaction mixture was cooled to −10° C. Molecular bromine (7.74 g, 48.4 mmol, 2.50 mL) was added dropwise followed by the slow addition of sodium nitrite (5.14 g, 74.5 mmol) in water (40 mL), maintaining the internal temperature below −10° C. The resulting mixture was stirred at room temperature for 18 hr. The reaction mixture was diluted with water (50 mL), neutralized to pH≈1 1-12 by the slow addition of NaOH and extracted with MTBE (2×30 mL). The combined organic layers were washed with water (30 mL), dried over Na₂SO₄ and concentrated in vacuo. The crude product was purified by distillation under reduced pressure (0.4 mbar, 45° C.) to give 3-bromo-2-cyclopropyl-pyridine (4.6 g, 23.2 mmol, 62.3% yield) as light-yellow liquid.

¹H NMR (500 MHz, CDCl₃) δ 1.01 (m, 2H), 1.08 (m, 2H), 2.51 (m, 1H), 6.89 (dd, 1H), 7.76 (dd, 1H), 8.34 (d, 1H).

GCMS: [M]⁺ m/z: calcd 196.98; found 196.0, 198.0; Rt=6.34.

### Step 2) 2-cyclopropyl-3-(4,4,5,5-tetramethyl-1,3,2-dioxaborolan-2-yl)pyridine

3-Bromo-2-cyclopropyl-pyridine (1.0 g, 5.05 mmol) and 4,4,5,5-tetramethyl-2-(4,4,5,5-tetramethyl-1,3,2-dioxaborolan-2-yl)-1,3,2-dioxaborolane (1.92 g, 7.57 mmol) were mixed in dioxane (8 mL). The mixture was evacuated and backfilled with argon. Bis(diphenylphosphino)ferrocene]dichloropalladium(II)-DCM (412 mg, 505 μmol) and potassium acetate (1.24 g, 12.6 mmol) were added to the mixture. The resulting mixture was heated at 90° C. for 18 hr. The reaction mixture was cooled to room temperature and concentrated under reduced pressure. The residue was diluted with water (10 mL) and extracted with EtOAc (3×10 mL). The combined organic layers were dried over anhydrous sodium sulfate and filtered through a pad of silica gel. The obtained filtrate was concentrated under reduce pressure to give 2-cyclopropyl-3-(4,4,5,5-tetramethyl-1,3,2-dioxaborolan-2-yl)pyridine (1.4 g, crude) as brown oil which was used in the next steps without further purification.

¹H NMR (500 MHz, CDCl₃) δ 0.95 (m, 2H), 1.09 (m, 2H), 1.34 (s, 12H), 2.85 (m, 1H), 6.98 (dd, 1H), 7.96 (d, 1H), 8.46 (m, 1H).

### Intermediate 33

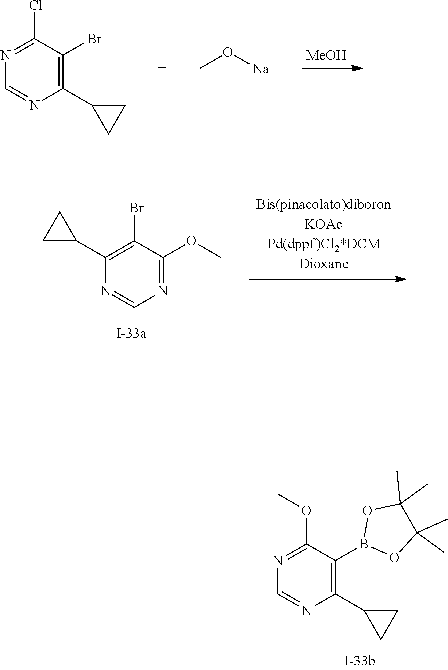

### Step a) 5-bromo-4-cyclopropyl-6-methoxy-pyrimidine

To a solution of 5-bromo-4-chloro-6-cyclopropyl-pyrimidine (20.0 g, 85.7 mmol) in MeOH (250 mL) sodium methoxide (4.63 g, 85.7 mmol) was added at 0° C. The resulting mixture was stirred at room temperature for 18 hr. The reaction mixture was concentrated under reduced pressure. The residue was diluted with water (200 mL) and the precipitate formed was collected by filtration to afford 5-bromo-4-cyclopropyl-6-methoxy-pyrimidine (19.0 g, 82.9 mmol, 96.83% yield) as a white solid which was used in the next steps without further purification.

¹H NMR (500 MHz, CDCl₃) δ 1.09 (m, 2H), 1.16 (m, 2H), 2.52 (m, 1H), 4.03 (s, 3H), 8.42 (s, 1H).

LCMS(ESI): [M+H]⁺ m/z: calcd 229; found 229.2.

### Step b) 4-cyclopropyl-6-methoxy-5-(4,4,5,5-tetramethyl-1,3,2-dioxaborolan-2-yl)pyrimidine

To a mixture of 5-bromo-4-cyclopropyl-6-methoxy-pyrimidine (19.0 g, 82.9 mmol), 4,4,5,5-tetramethyl-2-(4,4,5,5-tetramethyl-1,3,2-dioxaborolan-2-yl)-1,3,2-dioxaborolane (31.6 g, 124 mmol) and potassium acetate (28.5 g, 290 mmol) in dioxane (150 mL) bis(diphenylphosphino)ferrocene]dichloropalladium(II)-DCM (6.77 g, 8.29 mmol) was added in an inert atmosphere. The resulting mixture was stirred at 100° C. for 18 hr. The reaction mixture was cooled to room temperature and concentrated under reduced pressure. The obtained residue was diluted with water (100 mL) and EtOAc (300 mL). The organic phase was separated, washed with water (2×50 mL) and filtered through a pad of SiO₂. The mother liquor was concentrated under reduced pressure. The residue was subjected to flash-column chromatography (SiO₂, hexane/MTBE as an eluent) to afford 4-cyclopropyl-6-methoxy-5-(4,4,5,5-tetramethyl-1,3,2-dioxaborolan-2-yl)pyrimidine (6.5 g, 23.5 mmol, 28.4% yield) as a white powder.

¹H NMR (500 MHz, CDCl₃) δ 1.19 (m, 2H), 1.23 (m, 2H), 1.38 (s, 12H), 2.10 (m, 1H), 3.92 (s, 3H), 8.56 (s, 1H).

LCMS(ESI): [M+H]⁺ m/z: calcd 277.19; found 277.2.

### Intermediate 34

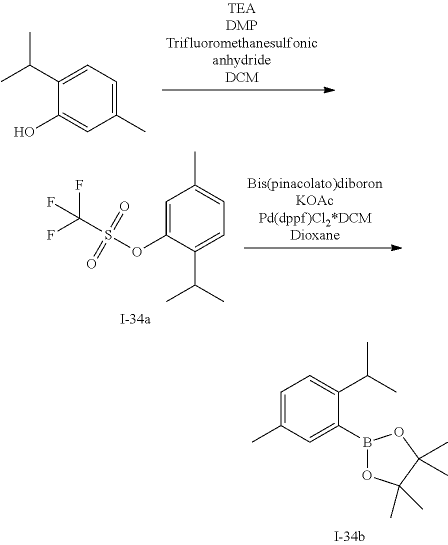

### Step a) (2-isopropyl-5-methyl-phenyl) trifluoromethanesulfonate

To a mixture of 2-isopropyl-5-methyl-phenol (1.0 g, 6.66 mmol) in DCM (50 mL) Triethylamine (1.35 g, 13.3 mmol, 1.86 mL) and 4-dimethylaminopyridine (41 mg, 332 μmol) were added. The reaction mixture was cooled to −50° C., and then trifluoromethanesulfonic anhydride (1.97 g, 6.99 mmol, 1.17 mL) was added dropwise. The resulting mixture was stirred at 25° C. for 24 hr. The reaction mixture was quenched with water (50 mL). The organic layer was separated and washed with saturated citric acid solution (10 mL), water (10 mL) and brine (10 mL), dried over anhydrous sodium sulfate and concentrated in vacuo to give (2-isopropyl-5-methyl-phenyl) trifluoromethanesulfonate (1.5 g, 5.31 mmol, 79.8% yield) as yellow oil which was used in the next steps without further purification.

¹H NMR (600 MHz, DMSO-d6) δ 1.19 (d, 6H), 2.31 (s, 3H), 3.10 (m, 1H), 7.12 (s, 1H), 7.28 (d, 1H), 7.44 (m, 1H).

### Step b) 2-(2-isopropyl-5-methyl-phenyl)-4,4,5,5-tetramethyl-1,3,2-dioxaborolane

(2-isopropyl-5-methyl-phenyl) trifluoromethanesulfonate (0.70 g, 2.48 mmol), potassium acetate (730.13 mg, 7.44 mmol), bis(diphenylphosphino)ferrocene]dichloropalladium(II)-DCM (9.0 mg, 124 μmol) and bis(pinacolato)diboron (945 mg, 3.72 mmol) were dissolved in dioxane (25 mL). The resulting mixture was evacuated and then backfilled with argon. This operation was repeated three times. The resulting mixture was stirred at 80° C. for 24 hr. The reaction mixture was cooled, diluted with water (25 mL) and extracted with EtOAc (50 mL). The organic layer was washed with water (20 mL) and brine (20 mL), dried over anhydrous sodium sulfate and concentrated in vacuo. The residue was subjected to HPLC (2-8 min 50-75% water−ACN; flow: 30 mL/min, column: Waters SunFire C18, 100×19 mm, 5 μm) to afford 2-(2-isopropyl-5-methyl-phenyl)-4,4,5,5-tetramethyl-1,3,2-dioxaborolane (0.15 g, 577 μmol, 23.3% yield) as colorless oil. ¹H NMR (400 MHz, DMSO-d6) δ 1.13 (d, 6H), 1.29 (s, 12H), 2.24 (s, 3H), 3.56 (m, 1H), 7.20 (m, 2H), 7.40 (s, 1H).

GCMS: [M]⁺ m/z: calcd 260.19; found 260.2.

### Intermediate 35

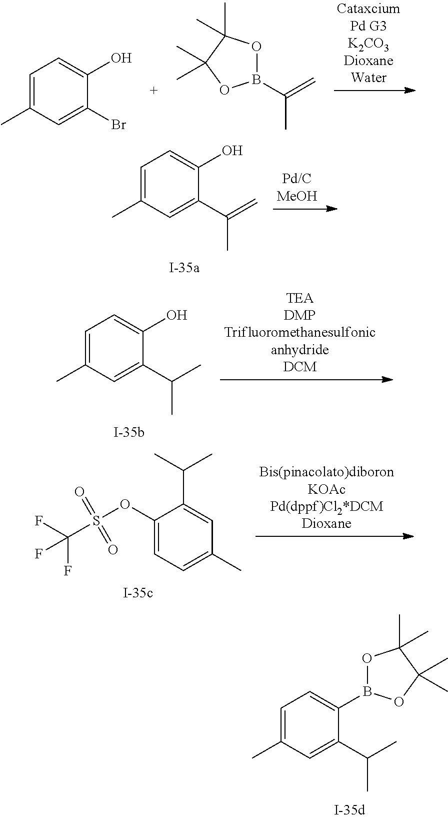

### Step a) 2-isopropenyl-4-methyl-phenol

2-bromo-4-methyl-phenol (6.00 g, 32.1 mmol), 2-isopropenyl-4,4,5,5-tetramethyl-1,3,2-dioxaborolane (8.09 g, 48.1 mmol), potassium carbonate (8.87 g, 64.2 mmol) and cataCXium® A Pd G3 (0.10 g, 641 μmol) were dissolved in dioxane (80 mL) and water (10 mL). The resulting mixture was evacuated and then backfilled with argon. This operation was repeated three times. The resulting mixture was stirred at 90° C. for 24 hr. The reaction mixture was cooled, diluted with EtOAc (120 mL) and water (80 mL). The organic layer was separated, washed with water (30 mL) and brine, dried over anhydrous sodium sulfate and concentrated in vacuo. The residue was subjected to flash-column chromatography (SiO₂; gradient Hexanes—MTBE) to give 2-isopropenyl-4-methyl-phenol (3.0 g, 20.2 mmol, 63.10%) as a light-yellow oil. Purity is 97% by LCMS but molar ion was not detected.

¹H NMR (400 MHz, DMSO-d₆) δ 2.05 (s, 3H), 2.17 (s, 3H), 5.04 (m, 2H), 6.69 (m, 1H), 6.87 (m, 2H), 9.11 (s, 1H).

### Step b) 2-isopropyl-4-methyl-phenol

2-isopropenyl-4-methyl-phenol (3.00 g, 20.2 mmol) was added to a suspension of palladium, 10% on carbon, (2.15 g) in MeOH (150 mL). The reaction mixture was evacuated and then backfilled with hydrogen. The resulting mixture was stirred in hydrogen atmosphere at 25° C. for 16 hr. The reaction mixture was filtered and the filtrate was concentrated under reduced pressure to give 2-isopropyl-4-methyl-phenol (2.80 g, 18.6 mmol, 92.1% yield) as light-yellow oil which was used in the next steps without further purification.

¹H NMR (500 MHz, DMSO-d₆) δ 1.10 (d, 6H), 2.15 (s, 3H), 3.13 (m, 1H), 6.60 (m, 1H), 6.73 (m, 1H), 6.87 (s, 1H), 8.91 (br, 1H).

### Step c) (2-isopropyl-4-methyl-phenyl) trifluoromethanesulfonate

To a solution of 2-isopropyl-4-methyl-phenol (2.80 g, 18.6 mmol) in DCM (100 mL) triethylamine (2.83 g, 28 mmol, 3.90 mL) and 4-dimethylaminopyridine (228 mg, 1.86 mmol) were added. The reaction mixture was cooled to −50° C., followed by the dropwise addition of trifluoromethanesulfonic anhydride (5.52 g, 19.6 mmol, 3.29 mL). The resulting mixture was stirred at 25° C. for 16 hr. The reaction mixture was quenched with water (100 mL). The organic layer was separated and washed sequentially with saturated citric acid solution (30 mL), water (50 mL) and brine (30 mL), dried over anhydrous sodium sulfate and concentrated in vacuo to give (2-isopropyl-4-methyl-phenyl) trifluoromethanesulfonate (4.50 g, 15.9 mmol, 85.5% yield) as a yellow oil which was used in the next steps without further purification.

¹H NMR (500 MHz, DMSO-d₆) δ 1.19 (d, 6H), 2.32 (s, 3H), 3.10 (m, 1H), 7.17 (m, 2H), 7.36 (s, 1H).

### Step d) 2-(2-isopropyl-4-methyl-phenyl)-4,4,5,5-tetramethyl-1,3,2-dioxaborolane

(2-isopropyl-4-methyl-phenyl) trifluoromethanesulfonate (5.70 g, 20.19 mmol), 4,4,5,5-tetramethyl-2-(4,4,5,5-tetramethyl-1,3,2-dioxaborolan-2-yl)-1,3,2-dioxaborolane (10.3 g, 40.4 mmol), potassium acetate (5.95 g, 60.6 mmol) and bis(diphenylphosphino)ferrocene]dichloropalladium(II)-DCM (0.30 g, 404 μmol) were mixed in dioxane (120 mL) in an inert atmosphere. The resulting mixture was evacuated and then backfilled with argon. This operation was repeated three times. The resulting mixture was stirred at 90° C. for 48 hr. The reaction mixture was cooled, diluted with water (100 mL) and extracted with EtOAc (100 mL). The organic layer was washed with water (2×50 mL) and brine (50 mL), dried over anhydrous sodium sulfate and concentrated in vacuo. The residue was purified by flash-column chromatography (SiO₂; gradient hexanes—MTBE) to afford 2-(2-isopropyl-4-methyl-phenyl)-4,4,5,5-tetramethyl-1,3,2-dioxaborolane (1.00 g, 3.84 mmol, 19.0% yield) as a white solid.

¹H NMR (400 MHz, CDCl₃) δ 1.23 (d, 6H), 1.35 (s, 12H), 2.35 (s, 3H), 3.69 (m, 1H), 6.99 (d, 1H), 7.13 (s, 1H), 7.65 (d, 1H).

LCMS(ESI): [M+H]⁺ m/z: calcd 261.24; found 261.2.

### Intermediate 36

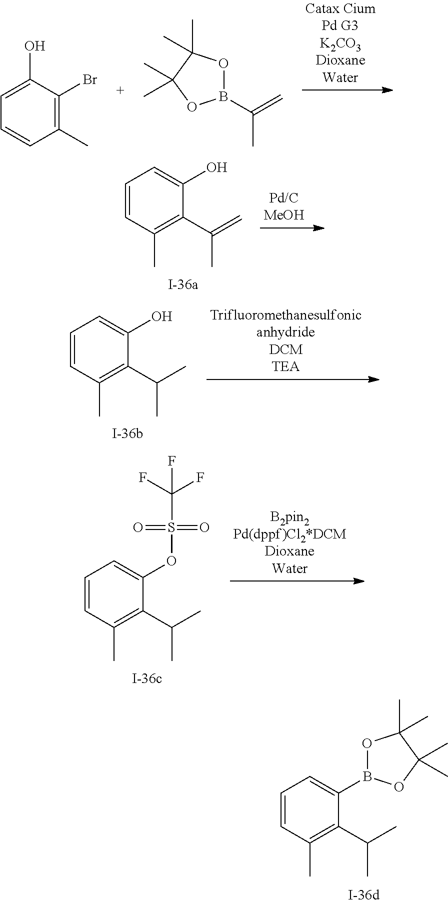

### Step a) 2-isopropenyl-3-methyl-phenol

A mixture of water (2 mL) and dioxane (8 mL) was evacuated and backfilled with argon, then 2-bromo-3-methyl-phenol (500 mg, 2.67 mmol), 2-isopropenyl-4,4,5,5-tetramethyl-1,3,2-dioxaborolane (674 mg, 4.01 mmol), cataCXium® A Pd G3 (146 mg, 200 μmol) and potassium carbonate (1.11 g, 8.02 mmol) were added in an inert atmosphere. The resulting mixture was stirred at 110° C. for 14 hr. The reaction mixture was cooled to room temperature and concentrated in vacuo. The residue was diluted with H₂O (30 mL) and extracted with EtOAc (3×50 mL). The combined organic layers were dried over anhydrous sodium sulfate and concentrated in vacuo. The residue was subjected to flash column chromatography (SiO₂; MTBE
n-Hexane, 1:9, Rf=0.5) to afford 2-isopropenyl-3-methyl-phenol (350 mg, 2.36 mmol, 88.34% yield) as a yellow oil.

¹H NMR (400 MHz, CDCl₃) δ 2.00 (s, 3H), 2.23 (s, 3H), 5.03 (s, 1H), 5.32 (br, 1H), 5.50 (s, 1H), 6.75 (m, 2H), 7.05 (m, 1H).

GCMS: [M]⁺ m/z: calcd 148.09; found 148.1.

### Step b) 3-methyl-2-(prop-1-en-2-yl)phenol

To a solution of 2-isopropenyl-3-methyl-phenol (350 mg, 2.36 mmol) in MeOH (5 mL) Pd, 10% on charcoal (25 mg) was added. The reaction mixture was hydrogenated under atmospheric pressure at 40° C. for 15 hr. and then filtered. The filtrate was concentrated in vacuo to afford 3-methyl-2-(prop-1-en-2-yl)phenol (310 mg, 2.06 mmol 87.3% yield) as a white solid which was used in the next steps without further purification.

¹H NMR (400 MHz, CDCl₃) δ 1.38 (d, 6H), 2.34 (s, 3H), 3.31 (m, 1H), 4.55 (br, 1H), 6.57 (d, 1H), 6.73 (d, 1H), 6.95 (m, 1H).

### Step c) (2-isopropyl-3-methyl-phenyl) trifluoromethanesulfonate

To a solution of 2-isopropyl-3-methyl-phenol (140 mg, 932 μmol) in DCM (10 mL) TEA (217 mg, 2.14 mmol) was added and the obtained mixture was cooled to 0° C. Trifluoromethanesulfonic anhydride (394 mg, 1.40 mmol, 235 μL) was added dropwise at 0° C. The resulting mixture was stirred for 14 hr. at ambient temperature. The reaction mixture was poured into H₂O (50 mL) and extracted with DCM (2×30 mL). The combined organic layers were washed with Brine (2×20 mL) dried over anhydrous sodium sulfate and concentrated in vacuo to afford (2-isopropyl-3-methyl-phenyl) trifluoromethanesulfonate (200 mg, 709 μmol, 76.0% yield) as a light-yellow oil which was used in the next steps without further purification. ¹H NMR (500 MHz, CDCl₃) δ 1.35 (d, 6H), 2.43 (s, 3H), 3.44 (m, 1H), 7.12 (m, 3H).

### Step d) 2-(2-isopropyl-3-methyl-phenyl)-4,4,5,5-tetramethyl-1,3,2-dioxaborolane

Dioxane (50 mL) was evacuated and backfilled with argon, then (2-isopropyl-3-methyl-phenyl) trifluoromethanesulfonate (2.30 g, 8.15 mmol), bis(pinacolato)diboron (5.17 g, 20.4 mmol) and bis(diphenylphosphino)ferrocene]dichloropalladium(II)-DCM (665 mg, 815 μmol) were added in an inert atmosphere. The resulting mixture was stirred at 100° C. for 12 hr. The reaction mixture was cooled to room temperature and concentrated in vacuo. The residue was diluted with H₂O (40 mL) and extracted with EtOAc (3×50 mL). The combined organic layers were dried over anhydrous sodium sulfate and concentrated in vacuo to give a crude product (2.5 g) which was purified by flash column chromatography (SiO₂; CHCl₃-n-Hexane, 1:1, Rf≈0.5) to afford 2-(2-isopropyl-3-methyl-phenyl)-4,4,5,5-tetramethyl-1,3,2-dioxaborolane (1.40 g, 5.38 mmol, yield is 66.0%) as a light-yellow oil.

¹H NMR (400 MHz, CDCl₃) δ 1.24 (m, 15H), 2.35 (s, 3H), 3.42 (m, 1H), 7.03 (m, 1H), 7.12 (m, 1H), 7.41 (m, 1H).

### Intermediate 37

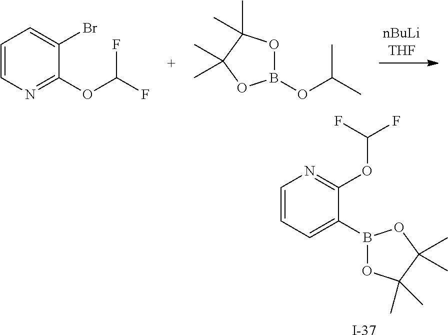

### Step a) 2-(difluoromethoxy)-3-(4,4,5,5-tetramethyl-1,3,2-dioxaborolan-2-yl)pyridine

To a solution of 3-bromo-2-(difluoromethoxy)pyridine (2.0 g, 8.93 mmol) and 2-isopropoxy-4,4,5,5-tetramethyl-1,3,2-dioxaborolane (3.32 g, 17.9 mmol) in THE (60 mL) n-Butyllithium, 2.5M in hexane, (5.36 mL, 13.4 mmol) was added dropwise in an inert atmosphere at −78° C. The reaction mixture was stirred at this temperature for 4 hr., then allowed to warm to room temperature and quenched with saturated aqueous NH₄C₁ (25 mL) solution. The obtained mixture was extracted with EtOAc (30 mL). The organic layer was separated, washed with water (20 mL), dried over anhydrous sodium sulfate and concentrated in vacuo to give 2-(difluoromethoxy)-3-(4,4,5,5-tetramethyl-1,3,2-dioxaborolan-2-yl)pyridine (2.5 g, crude) as light-yellow oil which was used in the next steps without further purification.

GCMS(ESI): [M]+ m/z: calcd 271.12; found 271.1.

### Intermediate 38

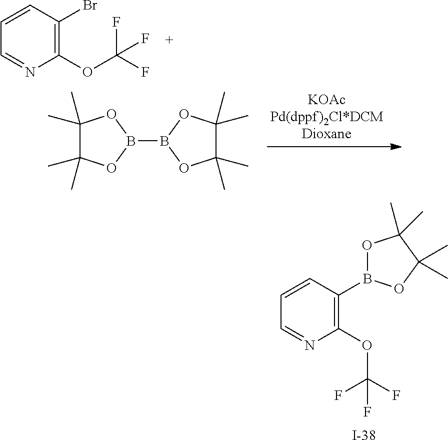

### Step a) 3-(4,4,5,5-tetramethyl-1,3,2-dioxaborolan-2-yl)-2-(trifluoromethoxy)pyridine

3-bromo-2-(trifluoromethoxy)pyridine (1.0 g, 4.13 mmol), 4,4,5,5-tetramethyl-2-(4,4,5,5-tetramethyl-1,3,2-dioxaborolan-2-yl)-1,3,2-dioxaborolane (1.57 g, 6.20 mmol), potassium acetate (1.01 g, 10.3 mmol) and bis(diphenylphosphino)ferrocene]dichloropalladium(II)-DCM (67.5 mg, 82.7 μmol) were sequentially added to degassed dioxane (20 mL). The resulting mixture was stirred at 100° C. for 24 hr. in an inert atmosphere. The reaction mixture was cooled, diluted with EtOAc (30 mL), washed with water (20 mL) and brine (20 mL). The organic phase was concentrated under reduced pressure to give 3-(4,4,5,5-tetramethyl-1,3,2-dioxaborolan-2-yl)-2-(trifluoromethoxy)pyridine (1.19 g, 100% yield) as brown oil which was used in the next step without further purification.

¹H NMR (400 MHz, CDCl₃) δ 1.34 (s, 12H), 7.19 (m, 1H), 8.10 (d, 1H), 8.36 (m, 1H).

GCMS: [M]+ m/z: calcd 289.11; found 289; Rt=6.54.

### Intermediate 39

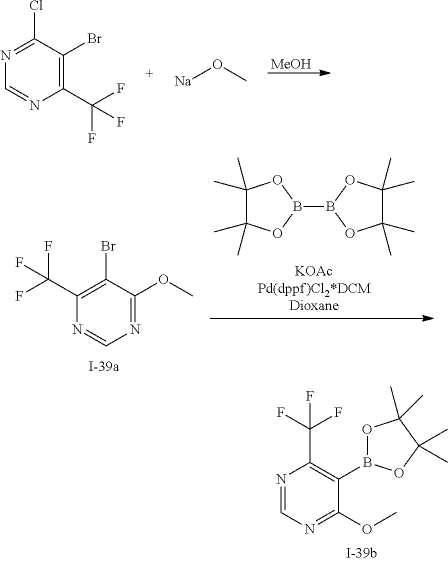

### Step a) 5-bromo-4-methoxy-6-(trifluoromethyl)pyrimidine

To a solution of 5-bromo-4-chloro-6-(trifluoromethyl)pyrimidine (1.5 g, 5.74 mmol) in MeOH (10 mL) methoxysodium (310 mg, 5.74 mmol) was added at 0° C. The resulting mixture was stirred for 18 hr. at room temperature. The reaction mixture was concentrated under reduced pressure. The residue was triturated with water (20 mL) and filtered off to give 5-bromo-4-methoxy-6-(trifluoromethyl)pyrimidine (850 mg, 3.31 mmol, 57.6% yield) as a white solid.

¹H NMR (500 MHz, CDCl₃) δ 4.12 (s, 3H), 8.73 (s, 1H).

### Step b) 4-methoxy-5-(4,4,5,5-tetramethyl-1,3,2-dioxaborolan-2-yl)-6-(trifluoromethyl)pyrimidine

To a mixture of 5-bromo-4-methoxy-6-(trifluoromethyl)pyrimidine (850 mg, 3.31 mmol), 4,4,5,5-tetramethyl-2-(4,4,5,5-tetramethyl-1,3,2-dioxaborolan-2-yl)-1,3,2-dioxaborolane (1.09 g, 4.30 mmol) and potassium acetate (974 mg, 9.92 mmol) in dioxane (15 mL) [1,1′-bis(diphenylphosphino)ferrocene]dichloropalladium(II)-DCM (270 mg, 330 μmol) was added under an inert atmosphere of argon. The reaction mixture was stirred at 100° C. for 18 hr. The reaction mixture was cooled to room temperature and concentrated under reduced pressure. The obtained residue was diluted with water (10 mL) and EtOAc (20 mL). The organic phase was separated, washed with water (2×10 mL) and filtered through a pad of SiO₂. The filtrate was concentrated under reduced pressure to give a crude product, which was purified by flash-column chromatography (SiO₂; gradient Hexane—MTBE) to obtain of 4-methoxy-5-(4,4,5,5-tetramethyl-1,3,2-dioxaborolan-2-yl)-6-(trifluoromethyl)pyrimidine (400 mg, 1.32 mmol, 39.8% yield) as light-yellow solid.

¹H NMR (500 MHz, CDCl₃) δ 1.38 (s, 12H), 4.03 (s, 3H), 8.86 (s, 1H).

LCMS(ESI): [M+H]⁺ m/z: calcd 305.12; found 305.1; Rt=3.29.

### Intermediate 40

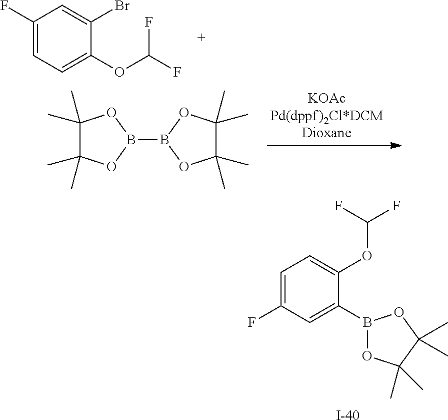

### Step a) 2-[2-(difluoromethoxy)-5-fluoro-phenyl]-4,4,5,5-tetramethyl-1,3,2-dioxaborolane

To a solution of 2-bromo-1-(difluoromethoxy)-4-fluoro-benzene (1.05 g, 4.36 mmol) in dioxane (10 mL) 4,4,5,5-tetramethyl-2-(4,4,5,5-tetramethyl-1,3,2-dioxaborolan-2-yl)-1,3,2-dioxaborolane (1.22 g, 4.79 mmol), potassium acetate (855 mg, 8.71 mmol) and bis(diphenylphosphino)ferrocene]dichloropalladium(II)-DCM (178 mg, 218 μmol) were sequentially added in an inert atmosphere. The resulting mixture was stirred at 80° C. for 16 hr. The reaction mixture was cooled to room temperature and concentrated under reduced pressure. The residue was diluted with EtOAc (40 mL), washed with brine (10 mL), dried over anhydrous sodium sulfate and concentrated in vacuo to afford 2-[2-(difluoromethoxy)-5-fluoro-phenyl]-4,4,5,5-tetramethyl-1,3,2-dioxaborolane (1.5 g, crude) as dark-brown oil which was used in the next steps without further purification.

¹H NMR (400 MHz, CDCl₃) δ 1.33 (s, 12H), 6.45 (t, 1H), 7.09 (m, 2H), 7.38 (m, 1H).

### Intermediate 41

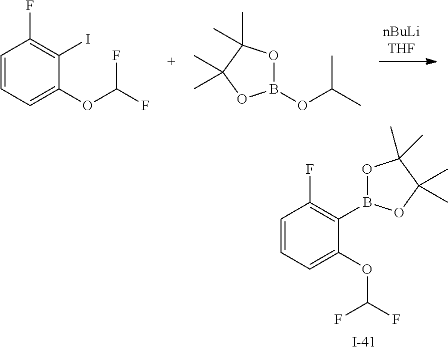

### Step a) 2-[2-(difluoromethoxy)-6-fluoro-phenyl]-4,4,5,5-tetramethyl-1,3,2-dioxaborolane

To a solution of 1-(difluoromethoxy)-3-fluoro-2-iodo-benzene (0.45 g, 1.56 mmol) in THE (5 mL) n-Butyllithium, 2.5M solution in hexane (0.81 mL, 2.03 mmol) was added dropwise at −80° C., followed by the addition of 2-isopropoxy-4,4,5,5-tetramethyl-1,3,2-dioxaborolane (436 mg, 2.34 mmol, 478 μL). The resulting mixture was stirred for 1 hr. at -80 to −30° C. The reaction mixture was quenched with water (5 mL) and extracted with hexane (10 mL). The organic phase was dried over anhydrous sodium sulfate and concentrated in vacuo to afford 2-[2-(difluoromethoxy)-6-fluoro-phenyl]-4,4,5,5-tetramethyl-1,3,2-dioxaborolane (0.20 g, 694 μmol, 44.4% yield) as yellow oil which was used in the next steps without further purification.

¹H NMR (400 MHz, CDCl₃) δ 1.36 (s, 12H), 6.48 (t, 1H), 6.89 (m, 2H), 7.33 (m, 1H).

### Intermediate 42

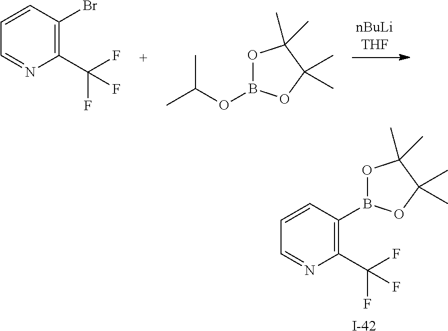

### Step a) synthesis of 3-(4,4,5,5-tetramethyl-1,3,2-dioxaborolan-2-yl)-2-(trifluoromethyl)pyridine

To a solution of 3-bromo-2-(trifluoromethyl)pyridine (1.49 g, 6.59 mmol) and 2-isopropoxy-4,4,5,5-tetramethyl-1,3,2-dioxaborolane (2.45 g, 13.2 mmol, 2.69 mL) in anhydrous THE (40 mL), n-butyl lithium, 2.5 M in hexane, (3.96 mL, 9.89 mmol) was added dropwise at −78° C. The resulting mixture was stirred for 4 hr. at −78° C. The reaction mixture was allowed to warm to room temperature and then quenched with saturated aqueous NH₄C₁ solution (20 mL). The organic phase was separated, dried over anhydrous magnesium sulfate and concentrated under reduced pressure to give 3-(4,4,5,5-tetramethyl-1,3,2-dioxaborolan-2-yl)-2-(trifluoromethyl)pyridine (2.2 g, crude) as a yellow liquid which was used in the next steps without further purification. ¹H NMR (400 MHz, CDCl₃) δ 1.36 (s. 12H), 7.43 (m, 1H), 8.02 (m, 1H), 8.70 (m, 1H).

LCMS(ESI): [M+H]⁺ m/z: calcd 274.14; found 274.0.

### Intermediate 43

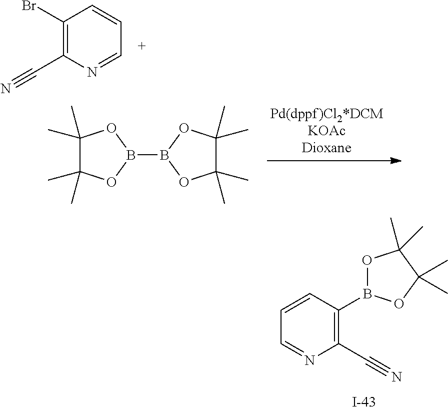

### Step a) 3-(4,4,5,5-tetramethyl-1,3,2-dioxaborolan-2-yl)pyridine-2-carbonitrile

3-bromopyridine-2-carbonitrile (1.50 g, 8.20 mmol), 4,4,5,5-tetramethyl-2-(4,4,5,5-tetramethyl-1,3,2-dioxaborolan-2-yl)-1,3,2-dioxaborolane (3.75 g, 14.8 mmol), potassium acetate (2.41 g, 24.6 mmol) and bis(diphenylphosphino)ferrocene]dichloropalladium(II)-DCM (134 mg, 164 μmol) were added to degassed dioxane (50 mL). The resulting mixture was stirred at 100° C. in an inert atmosphere for 12 hr. The reaction mixture was cooled to room temperature and diluted with EtOAc (100 mL). The obtained mixture was washed with water (50 mL) and brine (50 mL). The organic phase was dried over anhydrous sodium sulfate and concentrated under reduce pressure to afford 3-(4,4,5,5-tetramethyl-1,3,2-dioxaborolan-2-yl)pyridine-2-carbonitrile (2.5 g, crude) as red oil which was used in the next steps without further purification.

¹H NMR (400 MHz, CDCl₃) δ 1.36 (s, 12H), 7.45 (m, 1H), 8.15 (m, 1H), 8.71 (m, 1H).

### Intermediate 44

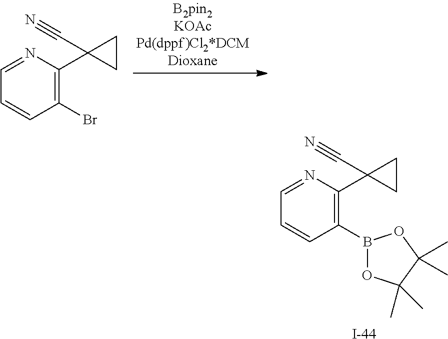

### Step a) I-[3-(4,4,5,5-tetramethyl-1,3,2-dioxaborolan-2-yl)-2-pyridyl]cyclopropanecarbonitrile

1-(3-bromo-2-pyridyl)cyclopropanecarbonitrile (1.50 g, 6.72 mmol), bis(pinacolato)diboron (1.88 g, 7.40 mmol) and potassium acetate (1.32 g, 13.45 mmol) were mixed in dioxane (10 mL). The reaction mixture was evacuated and then backfilled with Ar. Bis(diphenylphosphino)ferrocene]dichloropalladium(II)-DCM (548 mg, 672 μmol) was added. The resulting mixture was stirred at 90° C. for 12 hr. The reaction mixture was cooled, diluted with MTBE (100 mL), filtered through a pad of SiO₂ and washed with water (150 mL). The organic layer was separated, dried over anhydrous sodium sulfate and concentrated in vacuo to give 1-[3-(4,4,5,5-tetramethyl-1,3,2-dioxaborolan-2-yl)-2-pyridyl]cyclopropanecarbonitrile (1.80 g, crude) as a brown oil which was used in the next steps without further purification.

¹H NMR (400 MHz, CDCl₃) δ 1.23 (s, 12H), 1.65 (m, 4H), 7.25 (m, 1H), 8.11 (m, 1H), 8.53 (m, 1H).

LCMS(ESI): [M+H]⁺ m/z: calcd 271.18; found 271.2.

### Intermediate 45

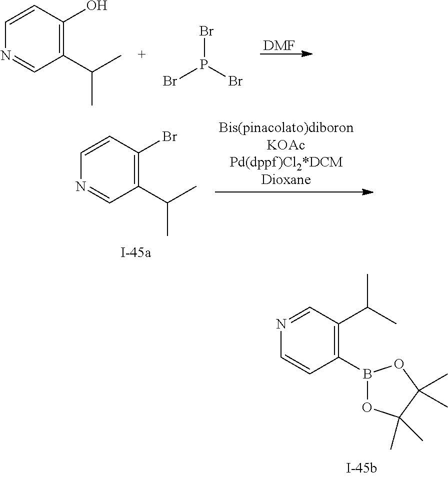

### Step a) 4-bromo-3-isopropyl-pyridine PP15C

To a solution of 3-isopropylpyridin-4-ol (5.0 g, 36.5 mmol) in DMF (5 mL) tribromophosphane (14.8 g, 54.7 mmol, 5.14 mL) was added. When bubble formation ceased, the suspension was poured into ice water under vigorous stirring. The obtained mixture was concentrated under reduced pressure. The residue was dissolved in saturated NaHCO₃ (20 mL) solution and extracted with MTBE (2×10 mL). The combined organic layers were dried over anhydrous sodium sulfate and concentrated under reduced pressure. The obtained residue was distilled under reduced pressure (0.40 mBar, 45° C.) to give 4-bromo-3-isopropyl-pyridine (3.2 g, 16.0 mmol, 43.9% yield) as light yellow liquid.

¹H NMR (500 MHz, CDCl₃) δ 1.29 (d, 6H), 3.32 (m, 1H), 7.44 (d, 1H), 8.21 (d, 1H), 8.46 (s, 1H).

LCMS(ESI): [M+H]⁺ m/z: calcd 199.0; found 199.

### Step b) 3-isopropyl-4-(4,4,5,5-tetramethyl-1,3,2-dioxaborolan-2-yl)pyridine

4-bromo-3-isopropyl-pyridine (3.2 g, 16.0 mmol), 4,4,5,5-tetramethyl-2-(4,4,5,5-tetramethyl-1,3,2-dioxaborolan-2-yl)-1,3,2-dioxaborolane (6.09 g, 24.0 mmol), potassium acetate (3.92 g, 40.0 mmol) and bis(diphenylphosphino)ferrocene]dichloropalladium(II)-DCM (1.31 g, 1.60 mmol) were dissolved in dioxane (8 mL) in an inert atmosphere. The resulting mixture was evacuated and then backfilled with argon. This operation was repeated three times. The resulting mixture was stirred at 90° C. for 18 hr. The reaction mixture was concentrated in vacuo. The residue was diluted with EtOAc (40 mL) and washed with water (3×15 mL). The organic phase was separated, dried over anhydrous sodium sulfate and concentrated under reduced pressure to obtain crude 3-isopropyl-4-(4,4,5,5-tetramethyl-1,3,2-dioxaborolan-2-yl)pyridine (2.5 g, crude) as a brown oil which was used in the next steps without further purification.

¹H NMR (400 MHz, CDCl₃) δ 1.21 (s, 12H), 1.33 (d, 6H), 2.92 (m, 1H), 7.25 (m, 1H), 8.41 (m, 2H).

### Intermediate 46

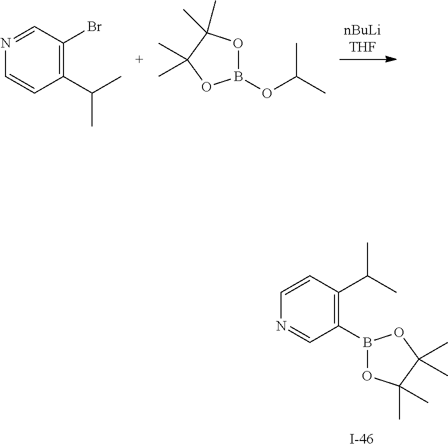

### Step a) 4-isopropyl-3-(4,4,5,5-tetramethyl-1,3,2-dioxaborolan-2-yl)pyridine

To a solution of 3-bromo-4-isopropyl-pyridine (1.00 g, 5.00 mmol) in dry THE (50 mL) n-butyllithium solution 2.5M in hexane (2.6 mL, 6.50 mmol) was added dropwise in an inert atmosphere at −78° C. The reaction mixture was stirred for 30 min at −78° C., then 2-isopropoxy-4,4,5,5-tetramethyl-1,3,2-dioxaborolane (1.39 g, 7.50 mmol, 1.53 mL) was added dropwise. The resulting mixture was stirred for 4 hr. gradually raising temperature to 20° C. The reaction mixture was quenched with water (10 mL), diluted with EtOAc (50 mL). The organic layer was separated, washed with water (10 mL) and brine (20 mL), dried over anhydrous sodium sulfate and concentrated under reduced pressure. The residue was subjected to HPLC (0.5-6.5 min 47% water—ACN; flow: 30 mL/min, column: Waters SunFire C18, 100×19 mm, 5 μm) to give 4-isopropyl-3-(4,4,5,5-tetramethyl-1,3,2-dioxaborolan-2-yl)pyridine (0.17 g, 688 μmol, 13.76% yield) as a light-yellow solid.

¹H NMR (400 MHz, DMSO-d₆) δ 1.17 (d, 6H), 1.32 (s, 12H), 3.54 (m, 1H), 7.33 (d, 1H), 8.53 (d, 1H), 8.65 (s, 1H).

### Intermediate 47

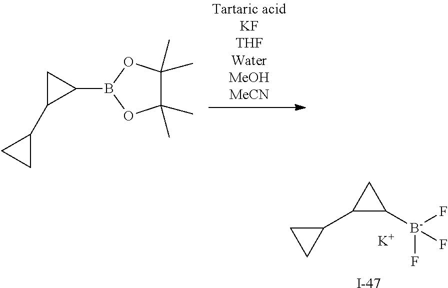

### Step a) (2-cyclopropylcyclopropyl)-trifluoro-boranuide

To a solution of 2-(2-cyclopropylcyclopropyl)-4,4,5,5-tetramethyl-1,3,2-dioxaborolane (400 mg, 1.92 mmol) in MeOH (4 mL) and MeCN (4 mL), KF (447 mg, 7.69 mmol) in water (8 mL) was added. The reaction mixture was stirred at 25° C. for 10 min. Tartaric acid (865 mg, 5.77 mmol) in THE (3 mL) was added to the reaction mixture. The resulting mixture was stirred for 0.1 hr. at room temperature. The reaction mixture was diluted with acetonitrile (5 mL). The mixture was filtered, the filtrate was concentrated in vacuo to give 150 mg of a crude product, which was triturated with CHCl₃ (10 mL). The precipitate was filtered off and dried in vacuo to obtain potassium [1,1′-bi(cyclopropan)]-2-yltrifluoroborate (170 mg, crude) as a white solid which was used in the next step as is.

¹H NMR (400 MHz, DMSO-d₆) 0.18-0.37 (m, 7H), 0.67 (m, 2H).

### Intermediate 48

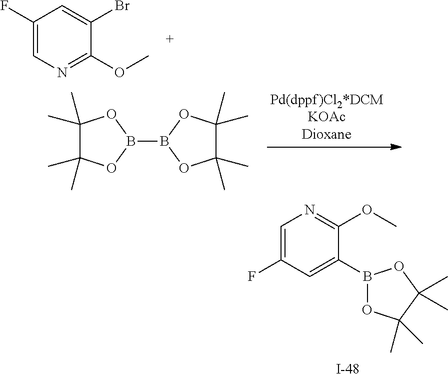

### Step a) 5-fluoro-2-methoxy-3-(4,4,5,5-tetramethyl-1,3,2-dioxaborolan-2-yl)pyridine

3-bromo-5-fluoro-2-methoxy-pyridine (500 mg, 2.43 mmol) was dissolved in dioxane (5 mL). The obtained mixture was evacuated and then backfilled with argon twice. 4,4,5,5-tetramethyl-2-(4,4,5,5-tetramethyl-1,3,2-dioxaborolan-2-yl)-1,3,2-dioxaborolane (924 mg, 3.64 mmol), bis(diphenylphosphino)ferrocene]dichloropalladium(II)-DCM (198 mg, 243 μmol) and potassium acetate (715 mg, 7.28 mmol, 455 μL) were added to the mixture. The resulting mixture was stirred at 90° C. for 10 hr. The reaction mixture was cooled to room temperature, diluted with EtOAc (5 mL) and hexane (5 mL), filtered and concentrated in vacuo to give 5-fluoro-2-methoxy-3-(4,4,5,5-tetramethyl-1,3,2-dioxaborolan-2-yl)pyridine (720 mg, crude) as brown oil which was used in the next steps without further purification.

¹H NMR (400 MHz, CDCl₃) δ 1.32 (s, 12H), 3.90 (s, 3H), 7.68 (m, 1H), 8.01 (m, 1H).

GCMS: [M] m/z: calcd 253.13; found 253.1.

### Intermediate 49

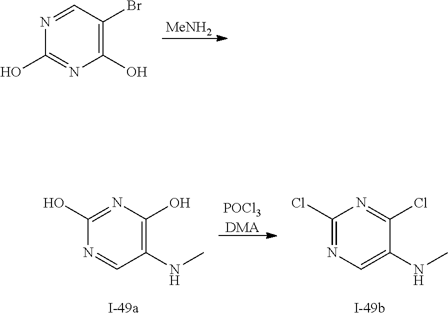

### Step a) 5-(methylamino)pyrimidine-2,4-diol

A mixture of 5-bromopyrimidine-2,4-diol (100 g, 522 mmol) and methylamine, 2M in methanol (556 mL) was stirred at 60° C. for 96 hr. The reaction mixture was concentrated in vacuo. The residue was diluted with ice cooled H₂O, the precipitate formed was filtered off and dried on air to afford 5-(methylamino)pyrimidine-2,4-diol (45.0 g, 319 mmol, 60.9% yield) as a light-yellow solid which was used in the next step without further purification.

¹H NMR (400 MHz, DMSO) δ 2.49 (s, 3H), 2.49 (br, 1H), 6.18 (s, 1H), 10.19 (br, 1H), 11.06 (br, 1H).

### Step b) 2,4-dichloro-N-methylpyrimidin-5-amine

5-(methylamino)pyrimidine-2,4-diol (45.0 g, 319 mmol) and phosphoryl chloride (175.8 g, 1.15 mol) were mixed together. To the obtained mixture dimethylaniline (17.6 g, 145 mmol, 18.4 mL) was slowly added at vigorous stirring. The mixture was stirred at 101° C. for 12 hr and then cooled to r.t. The reaction mixture was concentrated in vacuo. The residue was quenched with a saturated ice cooled aqueous NaHCO₃ solution (300 mL). The obtained mixture was basified with NaHCO₃ to pH≈7 and stirred for 40 min. The precipitate formed was filtered off. The filter cake was washed with H₂O and subjected to flash-column chromatography (SiO₂; CHCl₃-ACN) to afford 2,4-dichloro-N-methylpyrimidin-5-amine (26.0 g, 146 mmol, 45.8% yield) as a white solid.

¹H NMR (400 MHz, DMSO) δ 2.80 (s, 3H), 8.07 (s, 1H).

LCMS(ESI): [M+H]⁺ m/z: calcd 177.99; found 177.8; Rt=0.92.

### Intermediate 51

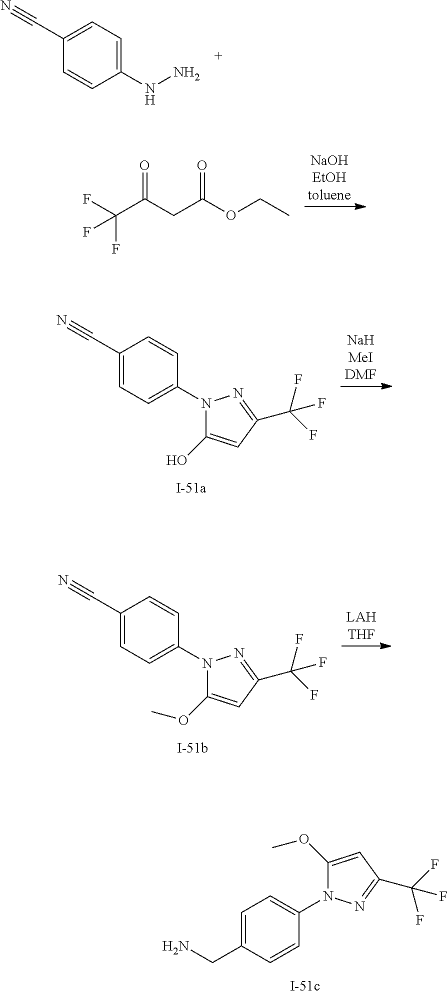 [SMILES: N#Cc1ccc(NN)cc1 ; CCOC(=O)CC(=O)C(F)(F)F ; N#Cc1ccc(-n2nc(C(F)(F)F)cc2O)cc1 ; COc1cc(C(F)(F)F)nn1-c1ccc(C#N)cc1 ; COc1cc(C(F)(F)F)nn1-c1ccc(CN)cc1]

### Step a: The synthesis of 4-(5-hydroxy-3-(trifluoromethyl)-1H-pyrazol-1-yl)benzonitrile

To a solution of 4-hydrazinobenzonitrile (4.00 g, 23.6 mmol, HCl salt) in EtOH (130 mL) Sodium hydroxide (943 mg, 23.6 mmol) was added. The obtained mixture was stirred at room temperature for 40 min, then ethyl 4,4,4-trifluoro-3-oxo-butanoate (5.21 g, 28.3 mmol, 4.14 mL) in EtOH (20.0 mL) was added. The resulting mixture was stirred under reflux for 24 hr. The reaction mixture was cooled to room temperature and filtered. The filtrate was concentrated in vacuo. The residue was dissolved in toluene (200 mL) and a catalytic amount of p-toluenesulfonic acid was added. The resulting mixture was stirred at 120° C. for 6 hr. The mixture was cooled to room temperature and concentrated in vacuo to afford 4-(5-hydroxy-3-(trifluoromethyl)-1H-pyrazol-1-yl)benzonitrile (4.60 g, 18.2 mmol, 77.04% yield) as a yellow solid which was used in the next steps without further purification.

¹H NMR (400 MHz, DMSO-d₆) δ 5.98 (s, 1H), 7.98 (s, 4H), 13.01 (br, 1H).

LCMS(ESI): [M+H]⁺ m/z: calcd 254.05; found 254.0.

### Step b: The synthesis of 4-(5-methoxy-3-(trifluoromethyl)-1H-pyrazol-1-yl)benzonitrile

To a solution of 4-(5-hydroxy-3-(trifluoromethyl)-1H-pyrazol-1-yl)benzonitrile (4.60 g, 18.2 mmol) in DMF (20.0 mL) Sodium hydride (479 mg, 19.9 mmol, 60% dispersion in mineral oil) was added at 0° C. The reaction mixture was stirred at room temperature for 2 hr. To the obtained mixture methyliodide (3.09 g, 21.8 mmol, 1.36 mL) was added dropwise. The resulting mixture was stirred at room temperature for 16 hr. The reaction mixture was poured into ice-water mixture (60 mL). The precipitate formed was filtered off and dissolved in EtOAc (30.0 mL). The obtained solution was washed with water (15.0 mL) and brine (15 mL), dried over anhydrous sodium sulfate and concentrated in vacuo to give a crude product (4.00 g) which was purified by recrystallization from hexane (30.0 mL) to afford 4-(5-methoxy-3-(trifluoromethyl)-1H-pyrazol-1-yl)benzonitrile (3.00 g, 11.2 mmol, 61.79%) as a yellow solid.

¹H NMR (500 MHz, DMSO-d₆) δ 4.04 (s, 3H), 6.54 (s, 1H), 7.91 (d, 2H), 8.00 (d, 2H).

LCMS(ESI): [M+H]⁺ m/z: calcd 268.07; found 268.0.

### Step c: The synthesis of (4-(5-methoxy-3-(trifluoromethyl)-1H-pyrazol-1-yl)phenyl)methanamine (U-51c)

To a suspension of lithium aluminium hydride (289 mg, 8.53 mmol) in THE (50.0 mL) a solution of 4-(5-methoxy-3-(trifluoromethyl)-1H-pyrazol-1-yl)benzonitrile (2.24 g, 7.11 mmol) in THE (5 mL) was added at 0° C. The reaction mixture was stirred at room temperature for 4 hr. The reaction mixture was cooled to 0° C. and quenched with water (1.00 mL). The solid was filtered out and the filtrate was concentrated under reduced pressure to give (4-(5-methoxy-3-(trifluoromethyl)-1H-pyrazol-1-yl)phenyl)methanamine (1.50 g, 5.53 mmol, 77.8% yield) as an yellow oil which was used in the next steps without further purification.

LCMS(ESI): [M+H]⁺ m/z: calcd 272.11; found 272.2.

### Intermediate 52

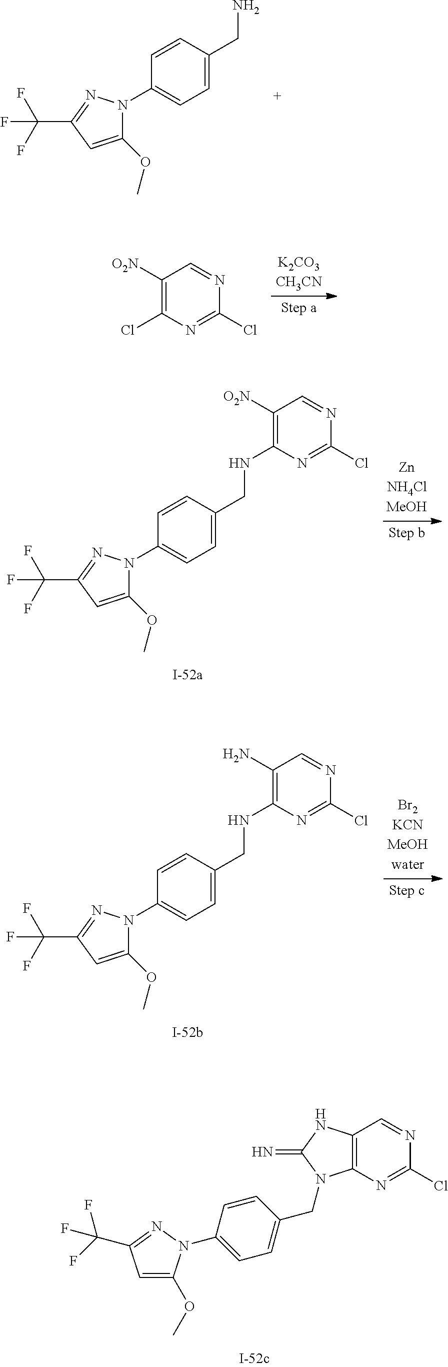 [SMILES: COc1cc(C(F)(F)F)nn1-c1ccc(CN)cc1 ; O=[N+]([O-])c1cnc(Cl)nc1Cl ; COc1cc(C(F)(F)F)nn1-c1ccc(CNc2nc(Cl)ncc2[N+](=O)[O-])cc1 ; COc1cc(C(F)(F)F)nn1-c1ccc(CNc2nc(Cl)ncc2N)cc1 ; COc1cc(C(F)(F)F)nn1-c1ccc(Cn2c(=N)[nH]c3cnc(Cl)nc32)cc1]

### Step a: The synthesis of 2-chloro-N-[[4-[5-methoxy-3-(trifluoromethyl)pyrazol-1-yl]phenyl]methyl]-5-nitro-pyrimidin-4-amine

The synthesis of the starting (4-(1-methyl-4-(trifluoromethyl)-1H-imidazol-2-yl)phenyl)methanamine is described in Intermediate 51.

Potassium carbonate (4.03 g, 29.2 mmol) was added to a stirred mixture of [4-[5-methoxy-3-(trifluoromethyl)pyrazol-1-yl]phenyl]methanamine (5.00 g, 19.4 mmol) and 2,4-dichloro-5-nitro-pyrimidine (3.77 g, 19.4 mmol) in ACN (300 mL). The mixture was stirred at ambient temperature for 16 hr. The reaction mixture was filtered. The filtrate was concentrated under reduced pressure to afford 2-chloro-N-[[4-[5-methoxy-3-(trifluoromethyl)pyrazol-1-yl]phenyl]methyl]-5-nitro-pyrimidin-4-amine (7.00 g, 16.3 mmol, 84.0% yield) as yellow solid which was used in a next step without further purifications.

LCMS(ESI): [M+H]⁺ m/z: calcd 429.07; found 429.0.

### Step b: The synthesis of 2-chloro-N₄-[[4-[5-methoxy-3-(trifluoromethyl)pyrazol-1-yl]phenyl]methyl]pyrimidine-4,5-diamine

To a stirred mixture of 2-chloro-N-[[4-[5-methoxy-3-(trifluoromethyl)pyrazol-1-yl]phenyl]methyl]-5-nitro-pyrimidin-4-amine (7.00 g, 16.3 mmol) and Ammonium Chloride (13.1 g, 245 mmol) in MeOH (500 mL) Zinc powder (8.54 g, 131 mmol) was added portionwise at -10-0 OC. The reaction mixture was stirred at room temperature for 14 hr. The reaction mixture was filtered. The filtrate was concentrated under reduced pressure. The residue was partitioned between DCM (600 mL) and water (250 mL). The organic layer was dried over anhydrous sodium sulfate and concentrated under reduced pressure to afford 2-chloro-N₄-[[4-[5-methoxy-3-(trifluoromethyl)pyrazol-1-yl]phenyl]methyl]pyrimidine-4,5-diamine (5.00 g, 12.5 mmol, 76.8%) as a light-yellow solid which was used in the next steps without further purification. LCMS(ESI): [M+H]⁺ m/z: calcd 399.11; found 399.0.

### Step c: The synthesis 2-chloro-9-[[4-[5-methoxy-3-(trifluoromethyl)pyrazol-1-yl]phenyl]methyl]-7H-purin-8-imine

BrCN (3.98 g, 37.6 mmol) was added portionwise to a solution of 2-chloro-N₄-[[4-[5-methoxy-3-(trifluoromethyl)pyrazol-1-yl]phenyl]methyl]pyrimidine-4,5-diamine (5.00 g, 12.5 mmol) in MeOH (250 mL) at room temperature. The reaction mixture was stirred at 40° C. for 72 hr. The mixture was cooled to room temperature and concentrated under reduced pressure. The residue was diluted with MTBE (200 mL). The solids were filtered off and partitioned between EtOAc (400 mL) and saturated aqueous NaHCO₃ solution (200 mL). The organic layer was separated, washed with brine (100 mL), dried over anhydrous Na₂SO₄ and concentrated under reduced pressure. The solvent was evaporated under the reduced pressure. The residue was subjected to flash-column chromatography (SiO₂, gradient acetonitrile—methanol) to afford 2-chloro-9-[[4-[5-methoxy-3-(trifluoromethyl)pyrazol-1-yl]phenyl]methyl]-7H-purin-8-imine (2.00 g, 4.72 mmol, 37.6% yield) as a light-yellow solid.

¹H NMR (500 MHz, DMSO-d₆) δ 3.96 (s, 3H), 5.32 (s, 2H), 6.42 (s, 1H), 7.34 (d, 2H), 7.45 (s, 1H), 7.60 (d, 2H), 8.29 (s, 1H).

LCMS(ESI): [M+H]⁺ m/z: calcd 424.09; found 424.0.

### Intermediate 53

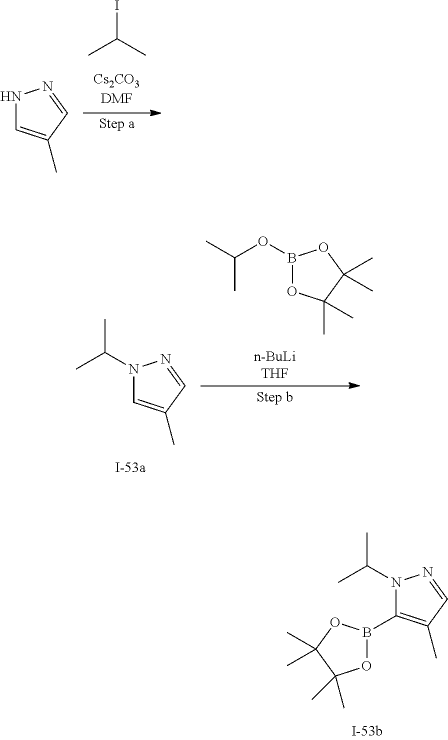

### Step a: The synthesis of I-isopropyl-4-methyl-pyrazole

A mixture of 4-methyl-1H-pyrazole (5.00 g, 60.9 mmol), isopropyl iodide (20.7 g, 122 mmol, 12.2 mL) and cesium carbonate (39.7 g, 122 mmol) in DMF (200 mL) was stirred at 80° C. for 12 hr. The reaction mixture was cooled to room temperature, poured into ice-cold water (300 mL) and extracted with MTBE (2×200 mL). The combined organic layers were washed with brine (100 mL), dried over anhydrous sodium sulfate and concentrated under reduced pressure to afford 1-isopropyl-4-methyl-pyrazole (6.20 g, 49.9 mmol, 82.0% yield) as a yellow liquid which was used in the next steps without further purification.

¹H NMR (500 MHz, CDCl₃) δ 1.43 (d, 6H), 2.03 (s, 3H), 4.34-4.43 (m, 1H), 7.15 (s, 1H), 7.26 (s, 1H).

LCMS(ESI): [M+H]⁺ m/z: calcd 125.13; found 125.0.

### Step b: The synthesis of 1-isopropyl-4-methyl-5-(4,4,5,5-tetramethyl-1,3,2-dioxaborolan-2-yl)pyrazole (I-53b)

n-Butyllithium (2.5 M, 40.0 mL) was added dropwise to a solution of 1-isopropyl-4-methyl-pyrazole (6.20 g, 49.9 mmol) in THE (120 mL) at −40° C. The reaction mixture was allowed to warm to 0° C. and stirred at this temperature for 1 hr. The reaction mixture was cooled to −78° C. 2-Isopropoxy-4,4,5,5-tetramethyl-1,3,2-dioxaborolane (18.6 g, 99.9 mmol, 20.4 mL) was added to the reaction mixture at −78° C. The resulting mixture was allowed to warm to room temperature and stirred at this temperature for 12 hr. The reaction mixture was quenched by dropwise addition of cold aqueous solution of NH₄C₁ (100 mL) and extracted with EtOAc (2×100 mL). The combined organic layers were washed with brine (100 mL), dried over anhydrous sodium sulfate and concentrated under reduced pressure. The residue was subjected to flash column chromatography (SiO₂, gradient hexane—EtOAc) to afford 1-isopropyl-4-methyl-5-(4,4,5,5-tetramethyl-1,3,2-dioxaborolan-2-yl)pyrazole (8.00 g, 32.0 mmol, 64.1% yield) as a yellow liquid.

¹H NMR (500 MHz, CDCl₃) δ 1.31 (s, 12H), 1.44 (d, 6H), 2.20 (s, 3H), 5.00-5.09 (m, 1H), 7.31 (s, 1H).

LCMS(ESI): [M+H]⁺ m/z: calcd 251.23; found 251.2.

### Intermediate 54

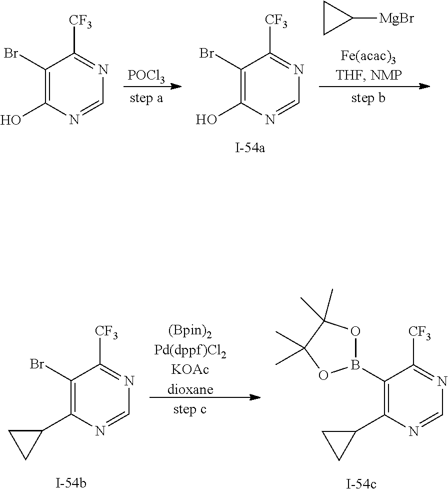

### Step a: Synthesis of 5-bromo-4-chloro-6-(trifluoromethyl)pyrimidine

5-bromo-6-(trifluoromethyl)pyrimidin-4-ol (4.00 g, 16.5 mmol) was mixed with phosphoryl chloride (5.05 g, 32.9 mmol). The resulting mixture was stirred at 101° C. for 6 hr. The mixture was cooled to room temperature and poured into water (150 mL). The resulting mixture was extracted with chloroform (2×80 mL). The combined organic layers were washed with water (50 mL) and brain (50 mL), dried over anhydrous sodium sulfate and concentrated under reduced pressure to afford 5-bromo-4-chloro-6-(trifluoromethyl)pyrimidine (4.00 g, 15.3 mmol, 92.94% yield) as a light-yellow oil which was used in the next steps without further purification.

¹H NMR (400 MHz, CDCl₃) δ 9.00 (s, 1H).

GCMS: [M]⁺ m/z: calcd 261.89 & 259.90; found 262 and 260.

### Step b: Synthesis of 5-bromo-4-cyclopropyl-6-(trifluoromethyl)pyrimidine

Cyclopropylmagnesium bromide (21.1 mmol, 17.5 mL, 1.21 M in THF) was added dropwise to a solution of tris(acetylacetonato)iron(III) (730 mg, 2.07 mmol) and 5-bromo-4-chloro-6-(trifluoromethyl)pyrimidine (2.70 g, 10.3 mmol) in tetrahydrofuran (15 mL) and N-Methyl-2-pyrrolidone (2.5 mL) at 0° C. The resulting mixture was allowed to warm to room temperature and stirred for 1 hr. The reaction mixture was quenched with water (100 mL) and extracted with EtOAc (100 mL). The organic layer was washed with brine (2×50 mL), dried over anhydrous sodium sulfate, and concentrated in vacuo. The residue was subjected to flash-column chromatography (SiO₂, gradient hexane-MTBE) to afford 5-bromo-4-cyclopropyl-6-(trifluoromethyl)pyrimidine (1.40 g, 5.24 mmol, 50.8% yield) as a white solid.

¹H NMR (400 MHz, CDCl₃) δ 1.20-1.39 (m, 4H), 2.69-2.81 (m, 1H), 8.94 (s, 1H).

### Step c: Synthesis of 4-cyclopropyl-5-(4,4,5,5-tetramethyl-1,3,2-dioxaborolan-2-yl)-6-(trifluoromethyl)pyrimidine (I-53c)

To a mixture of 4,4,5,5-tetramethyl-2-(4,4,5,5-tetramethyl-1,3,2-dioxaborolan-2-yl)-1,3,2-dioxaborolane (357 mg, 1.40 mmol), 5-bromo-4-cyclopropyl-6-(trifluoromethyl)pyrimidine (250 mg, 936.17 μmol) and potassium acetate (276 mg, 2.81 mmol) in degasses Dioxane (10 mL) bis(diphenylphosphino)ferrocene]dichloropalladium(II)-DCM (76.5 mg, 93.6 μmol) was added under argon atmosphere. The resulting mixture was stirred at 95° C. for 20 hr. The mixture was cooled to room temperature and concentrated under reduced pressure. The residue was submitted to flash-column chromatography (SiO₂, gradient hexane-MTBE) to afford 4-cyclopropyl-5-(4,4,5,5-tetramethyl-1,3,2-dioxaborolan-2-yl)-6-(trifluoromethyl)pyrimidine (330 mg, crude) as a brown liquid which was used in the next steps without further purification.

LCMS(ESI): [M+H]⁺ m/z: calcd 315.18; found 315.2.

### Intermediate 55

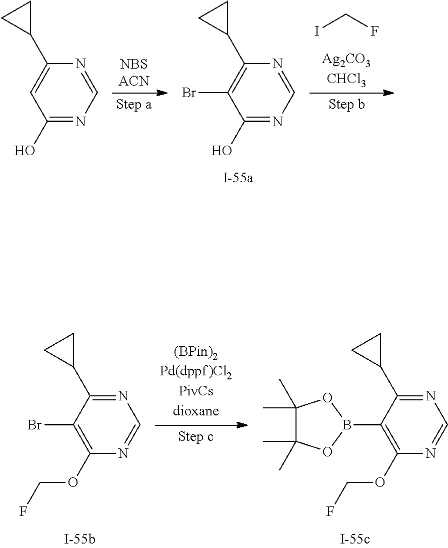

### Step a: The Synthesis of 5-bromo-6-cyclopropyl-pyrimidin-4-ol

N-Bromosuccinimide (2.75 g, 15.4 mmol) was added portionwise to a solution of 6-cyclopropylpyrimidin-4-ol (2.00 g, 14.7 mmol) in ACN (14.7 mL) at room temperature. The reaction mixture was stirred at room temperature for 1 hr. The reaction mixture was concentrated under reduced pressure. The residue was diluted with aqueous thiosulfate (5% wt.). The resulting mixture was stirred for 10 min. The solid precipitate was filtered off, washed with water and dried on air to afford 5-bromo-6-cyclopropyl-pyrimidin-4-ol (2.60 g, 12.1 mmol, 82.3% yield) as a light-yellow solid which was used in the next steps without further purification.

¹H NMR (500 MHz, DMSO-d₆) δ 0.95-1.05 (m, 4H), 2.31-2.35 (m, 1H), 8.04 (s, 1H), 12.75 (br, 1H).

LCMS(ESI): [M+H]⁺ m/z: calcd 214.98; found 215.0.

### Step b: The synthesis of 5-bromo-4-cyclopropyl-6-(fluoromethoxy)pyrimidine

5-bromo-6-cyclopropyl-pyrimidin-4-ol (900 mg, 4.19 mmol), fluoroiodomethane (3.00 g, 18.8 mmol) and silver carbonate (1.05 g, 6.28 mmol) were mixed in chloroform (5.0 mL). The reaction mixture was stirred at 50° C. for 72 hr. The reaction mixture was cooled to room temperature and filtered. The filtrate was concentrated under reduced pressure. The residue was subjected to flash column chromatography (SiO₂, gradient dichloromethane—EtOAc) to afford 5-bromo-4-cyclopropyl-6-(fluoromethoxy)pyrimidine (350 mg, 1.42 mmol, 33.9% yield) as a yellow solid.

¹H NMR (500 MHz, CDCl₃) δ 1.12-1.16 (m, 2H), 1.19-1.23 (m, 2H), 2.54-2.60 (m, 1H), 6.11 (d, 2H, CH₂F), 8.49 (s, 1H).

LCMS(ESI): [M+H]⁺ m/z: calcd 247.00; found 247.0.

### Step c: The synthesis of 4-cyclopropyl-6-(fluoromethoxy)-5-(4,4,5,5-tetramethyl-1,3,2-dioxaborolan-2-yl)pyrimidine

Bis(diphenylphosphino)ferrocene]dichloropalladium(II)-DCM (33.0 mg, 40.5 μmol) was added to a mixture of 5-bromo-4-cyclopropyl-6-(fluoromethoxy)pyrimidine (200 mg, 810 μmol), cesium pivalate (322 mg, 1.38 mmol) and bis(pinacolato)diboron (308 mg, 1.21 mmol) in degassed dioxane (3.0 mL) under argon atmosphere. The reaction mixture was stirred at 75° C. for 12 hr. The reaction mixture was cooled to room temperature and filtered through a pad of silica gel. The filtrate was concentrated under reduced pressure to afford 4-cyclopropyl-6-(fluoromethoxy)-5-(4,4,5,5-tetramethyl-1,3,2-dioxaborolan-2-yl)pyrimidine (700 mg, crude) as a yellow oil which as used in the next step without further purification.

LCMS(ESI): [M+H]⁺ m/z: calcd 295.17; found 295.2.

### Intermediate 56

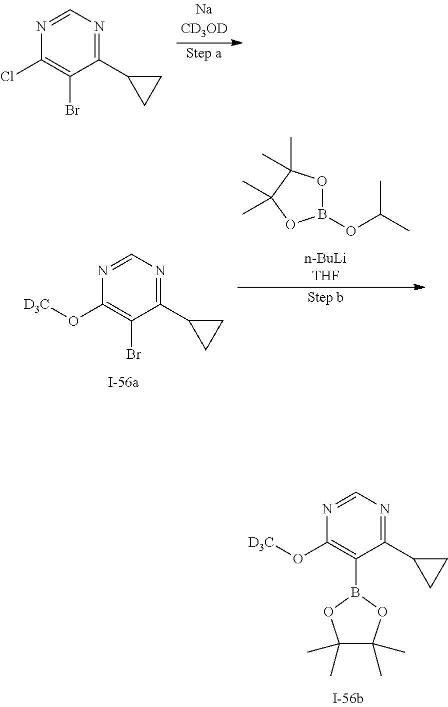 [SMILES: Clc1ncnc(C2CC2)c1Br ; CC(C)OB1OC(C)(C)C(C)(C)O1 ; [2H]C([2H])([2H])Oc1ncnc(C2CC2)c1Br ; [2H]C([2H])([2H])Oc1ncnc(C2CC2)c1B1OC(C)(C)C(C)(C)O1]

### Step a: The synthesis of 5-bromo-4-cyclopropyl-6-(trideuteriomethoxy)pyrimidine

Sodium (325 mg, 14.1 mmol) was added portionwise to vigorously stirred trideuterio(deuteriooxy)methane (23.2 g, 642 mmol, 26.0 mL). The resulting mixture was stirred at room temperature for 1 hr, then cooled to 0° C. 5-bromo-4-chloro-6-cyclopropyl-pyrimidine (3.00 g, 12.9 mmol) was added to the mixture. The reaction mixture was stirred at room temperature for 16 hr. The reaction mixture was concentrated under reduced pressure. The residue was diluted with EtOAc (150 mL) and washed with water (30 mL). The organic layer was separated, washed with brine (20 mL), dried over anhydrous sodium sulfate and concentrated under reduced pressure to afford 5-bromo-4-cyclopropyl-6-(trideuteriomethoxy)pyrimidine (2.80 g, 12.1 mmol, 93.9% yield) as a yellow solid which was used in the next steps without further purification.

¹H NMR (500 MHz, CDCl₃) δ 1.05-1.10 (m, 2H), 1.14-1.20 (m, 2H), 2.47-2.55 (m, 1H), 8.42 (s, 1H).

LCMS(ESI): [M+H]⁺ m/z: calcd 232.02; found 232.2.

### Step b: The synthesis of 4-cyclopropyl-5-(4,4,5,5-tetramethyl-1,3,2-dioxaborolan-2-yl)-6-(trideuteriomethoxy)pyrimidine

5-bromo-4-cyclopropyl-6-(trideuteriomethoxy)pyrimidine (2.80 g, 12.1 mmol) and 2-isopropoxy-4,4,5,5-tetramethyl-1,3,2-dioxaborolane (3.14 g, 16.9 mmol, 3.45 mL) were mixed in THE (100 mL) under argon atmosphere. The resulting solution was cooled to −80° C. n-Butyllithium (18.1 mmol, 7.24 mL, 2.5 M in hexane) was added dropwise to the solution at -80° C. The reaction mixture was stirred at −80° C. for 3 hr, then at ambient temperature for 16 hr. The reaction mixture was quenched with a saturated aqueous solution of NH₄C₁ (20 mL) and extracted with EtOAc (30 mL). The organic layer was separated, washed with water (20 mL) and brine (30 mL), dried over anhydrous sodium sulfate and concentrated under reduced pressure. The residue was subjected to flash column chromatography (SiO₂, gradient hexane—MTBE) to afford 4-cyclopropyl-5-(4,4,5,5-tetramethyl-1,3,2-dioxaborolan-2-yl)-6-(trideuteriomethoxy)pyrimidine (800 mg, 2.87 mmol, 23.8% yield) as a white solid.

¹H NMR (500 MHz, CDCl₃) δ 0.93-1.00 (m, 2H), 1.13-1.19 (m, 2H), 1.38 (s, 12H), 2.04-2.10 (m, 1H), 8.55 (s, 1H).

LCMS(ESI): [M+H]⁺ m/z: calcd 280.19; found 280.2.

### Example A-1

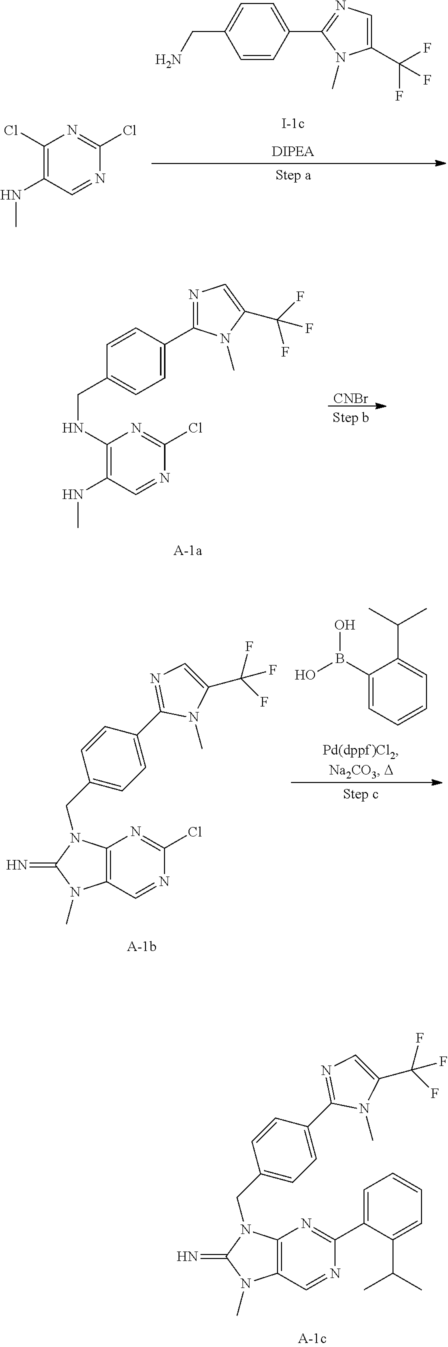

### Step a) 2-chloro-N5-methyl-N4-(4-(1-methyl-5-(trifluoromethyl)-1H-imidazol-2-yl)benzyl)pyrimidine-4,5-diamine (A-1a)

DIPEA (0.93 mL, 0.6 mmol) was added to a stirred solution of 2,4-dichloro-N-methylpyrimidin-5-amine (50 mg, 0.28 mmol) and compound I-1c (72 mg, 2.93 mmol) in THE (10 mL) at 0° C. The resulting mixture was stirred for 12 h at 80° C., then water (10 mL) was added and the mixture was extracted with EtOAc (2×10 mL). The organic layer was washed with water, brine, dried (Na₂SO₄), filtered and concentrated under reduced pressure, which gave the title compound (100 mg) as a liquid. MS (ES+) 397.33 [M+H]⁺. The compound was taken to next step without further purification.

### Step b) 2-chloro-7-methyl-9-(4-(1-methyl-5-(trifluoromethyl)-1H-imidazol-2-yl)benzyl)-7H-purin-8(9H)-imine (A-1b)

Cyanogen bromide (1.1 g, 10.1 mmol) was added at 0° C. to a stirred solution of compound A-1a (1 g, 2.52 mmol) in EtOH (50 mL). The resulting mixture was stirred at rt for 30 min followed by 12 h at 80° C., then concentrated, which gave the title compound (1 g) as a solid.

MS (ES+) 422.40 [M−H]⁻. The compound was taken to next step without further purification.

### Step c) 2-(2-isopropylphenyl)-7-methyl-9-(4-(J-methyl-5-(trifluoromethyl)-1H-imidazol-2-yl)benzyl)-7H-purin-8(9H)-imine (A-1c)

Sodium carbonate (628 mg, 5.93 mmol) and (2-isopropylphenyl) boronic acid (500 mg, 3.1 mmol) were added to a stirred solution of compound A-1b (1 g, 0.71 mmol) in 1,4-dioxane (20 mL) and water (7 mL) in a sealed tube. The reaction mixture was degassed by bubbling with argon for 10 min then Pd(dppf)Cl₂·DCM, (194 mg, 0.24 mmol) was added and the reaction mixture was stirred at 100° C. for 16 h in a sealed tube. The reaction mixture was concentrated and diluted with water (10 mL), extracted with EtOAc (3×25 mL) and the combined organic layers were washed with brine, dried (Na₂SO₄) and concentrated under reduced pressure. The afforded crude compound was triturated with pentane and purified by prep HPLC Sunfire C₁₈ column (30×150) mm 5 u using a gradient of 10 mM NH₄HCO₃ in H₂O: MeCN as mobile phase.

The impure compound was further purified by SFC, which gave the title compound (100 mg, 8%) as a solid. LCMS (ES+) m/z 506.52 [M+H]⁺.

¹H NMR (500 MHz, DMSO): δ 8.23 (s, 1H), 7.66 (d, J=8.2 Hz, 2H), 7.61 (d, J=1.1 Hz, 1H), 7.49 (q, J=5.0 Hz, 3H), 7.37 (m, J=3.6 Hz, 2H), 7.22 (m, J=3.2 Hz, 1H), 6.53 (d, J=11.8 Hz, 1H), 5.18 (s, 2H), 3.71 (s, 3H), 3.44 (q, J=6.8 Hz, 1H), 3.39 (s, 3H), 1.08 (d, J=6.9 Hz, 6H).

Preparative SFC Conditions
Column/dimensions: Chiralcel OD-H (250×30 mm), 5μ
CO₂:75.0%
Co solvent: 25.0% (30 mM methanolic ammonia in methanol)
Total flow: 70.0 g/min
Back pressure: 90.0 bar
UV: 214 nm
Stack time: 5.1 min
Load/Inj.: 7.0 mg

### Example A-2

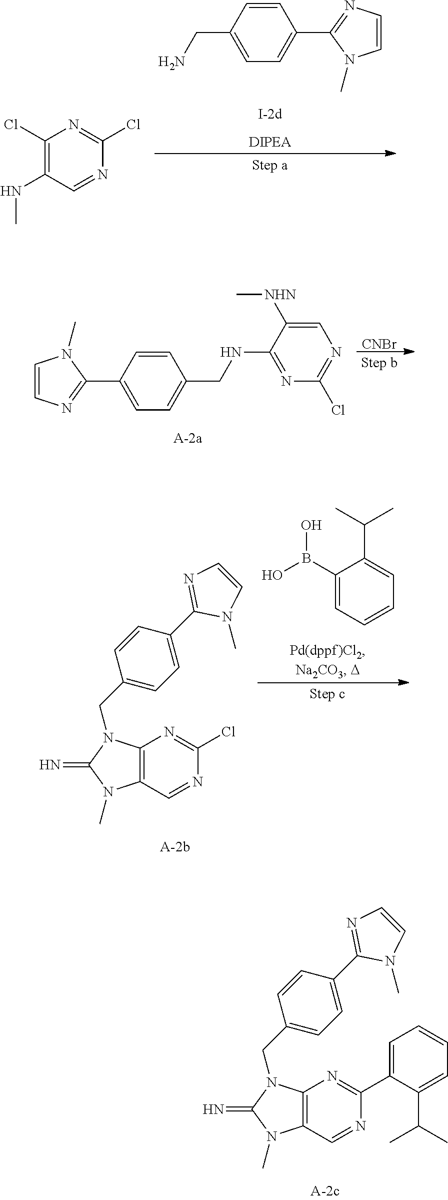

### Step a) 2-chloro-N5-methyl-N4-(4-(1-methyl-1H-imidazol-2-yl)benzyl)pyrimidine-4,5-diamine (A-2a)

DIPEA (2.9 mL, 16.5 mmol) was added to a stirred solution of 2,4-dichloro-N-methylpyrimidin-5-amine (508 mg, 2.75 mmol) and compound I-2d (700 mg, 2.75 mmol) in THE (50 mL) at rt. The resulting mixture was stirred for 120 h at 80° C., then concentrated. Ice cold water (40 mL) was added and the mixture was extracted with EtOAc (2×50 mL). The organic layer was washed with brine, dried (Na₂SO₄), filtered and concentrated under reduced pressure, which gave the title compound (500 mg) as a liquid. MS (ES+) 329.35 [M+H]⁺. The compound was taken to next step without further purification.

### Step b) 2-chloro-7-methyl-9-(4-(1-methyl-1H-imidazol-2-yl)benzyl)-7H-purin-8(9H)-imine (A-2b)

Cyanogen bromide (466 mg, 4.4 mmol) was added at 0° C. to a stirred solution of compound A-2a (500 mg, 1.1 mmol) in EtOH (25 mL). The resulting mixture was stirred at 80° C. for 6 h, then concentrated, which gave the title compound (520 mg) as a semi-solid. MS (ES+) 354.39 [M+H]⁺. The compound was taken to next step without further purification.

### Step c)₂-(2-isopropylphenyl)-7-methyl-9-(4-(1-methyl-1H-imidazol-2-yl)benzyl)-7H-purin-8(9H)-imine (A-2c)

Sodium carbonate (93 mg, 0.9 mmol) and (2-isopropylphenyl) boronic acid (58 mg, 0.4 mmol) were added to a stirred solution of compound A-2b (500 mg, 0.30 mmol) in 1,4-dioxane (20 mL) and water (10 mL) in a sealed tube. The reaction mixture was degassed by bubbling with argon for 10 minutes then Pd(dppf)Cl₂·DCM, (48 mg, 0.06 mmol) was added and the reaction mixture was stirred at 110° C. for 16 h in a sealed tube. The reaction mixture was diluted with water (10 mL), extracted with EtOAc (3×25 mL). The combined organic layers were washed with brine, dried (Na₂SO₄), filtered and concentrated under reduced pressure. The crude compound was triturated with 30% diethyl ether in pentane (2×10 mL). The afforded residue was purified by prep HPLC Sunfire C₁₈ column (30×150) mm 5p using a gradient of 10 mM NH₄HCO₃ in H₂O: MeCN as mobile phase, which gave the title compound (35 mg, 27%) as a solid. LCMS (ES+) m/z 438.47 [M+H]⁺.

¹H NMR (500 MHz, DMSO): δ 8.24 (s, 1H), 7.63 (d, J=8.3 Hz, 2H), 7.48 (q, J=3.0 Hz, 1H), 7.43 (d, J=8.3 Hz, 2H), 7.38 (m, J=3.4 Hz, 2H), 7.23 (q, J=3.4 Hz, 2H), 6.94 (d, J=1.1 Hz, 1H), 6.60 (s, 1H), 5.15 (s, 2H), 3.71 (s, 3H), 3.44 (t, J=6.9 Hz, 1H), 3.39 (s, 3H), 1.09 (d, J=6.9 Hz, 6H).

### Example A-3

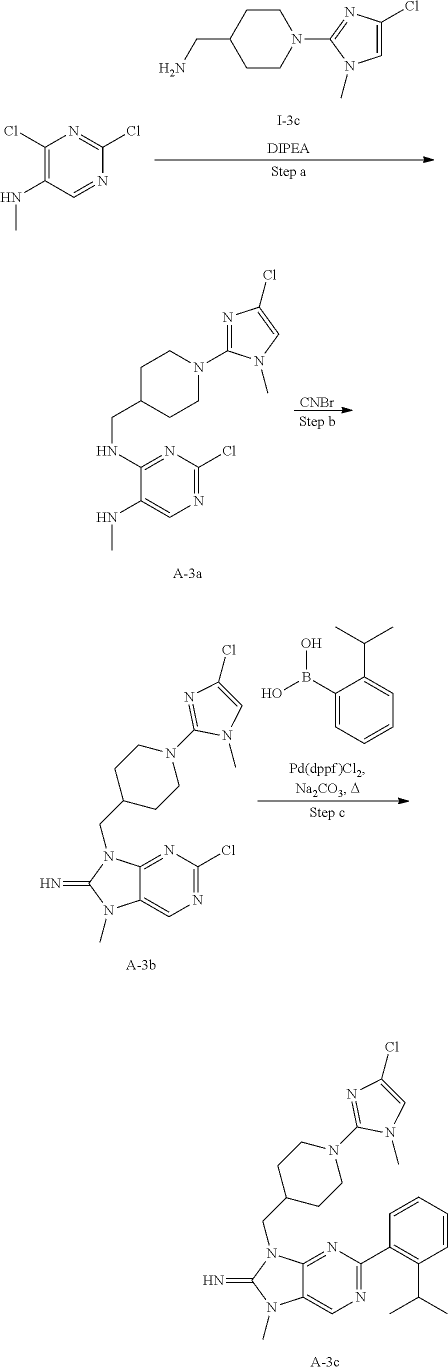

### Step a) 2-chloro-N4-((1-(4-chloro-1-methyl-1H-imidazol-2-yl)piperidin-4-yl)methyl)-N₅-methylpyrimidine-4,5-diamine (A-3a)

DIPEA (0.81 mL, 5.0 mmol) was added to a stirred solution of 2,4-dichloro-N-methylpyrimidin-5-amine (455 mg, 2.50 mmol) and compound I-3c (600 mg, 2.0 mmol) in THE (15 mL) at 0° C. and stirred at rt for 30 min. The mixture was heated at 70° C. for 48 h, then concentrated under reduced pressure. Water (40 mL) was added and the mixture was extracted with EtOAc (2×50 mL). The organic layer was washed with brine, dried (Na₂SO₄), filtered and concentrated under reduced pressure. The crude compound was purified by column chromatography on silica gel and eluted with 80% EtOAc/pet ether, which gave the title compound (300 mg, 32%) as a solid. LCMS (ES+) 370.39 [M+H]⁺.

### Step b) 2-chloro-9-((J-(4-chloro-1-methyl-1H-imidazol-2-yl)piperidin-4-yl)methyl)-7-methyl-7H-purin-8(9H)-imine (A-3b)

Cyanogen bromide (160 mg, 1.52 mmol) was added at 0° C. to a stirred solution of compound A-3a (300 mg, 0.76 mmol) in EtOH (20 mL). The resulting mixture was stirred at rt for 30 min and heated at 80° C. for 2 h, then concentrated under reduced pressure. The residue was basified with saturated NaHCO₃ solution. The aqueous layer was extracted with EtOAc (2×25 mL). The combined organic layers were washed with brine, dried (Na₂SO₄), filtered and concentrated under reduced pressure, which gave the crude title compound (350 mg) as a liquid. The compound was taken to next step without further purification.

### Step c) 9-((1-(4-chloro-1-methyl-1H-imidazol-2-yl)piperidin-4-yl)methyl)-2-(2-isopropylphenyl)-7-methyl-7H-purin-8(9H)-imine (A-3c)

Sodium carbonate (155 mg, 1.50 mmol) and (2-isopropylphenyl) boronic acid (144 mg, 0.90 mmol) were added to a stirred solution of compound A-3b (350 mg, 0.30 mmol) in 1,4-dioxane (12 mL) and water (3 mL) in a sealed tube. The reaction mixture was degassed by bubbling with argon for 10 minutes then Pd(dppf)Cl₂·DCM (64 mg, 0.1 mmol) was added and the reaction mixture was stirred at 100° C. for 16 h in a sealed tube. The reaction mixture was concentrated, diluted with water (10 mL), extracted with EtOAc (3×25 mL) and the combined organic layers were dried (Na₂SO₄) and concentrated under reduced pressure. The crude compound was purified by column chromatography on silica gel and eluted with 10% MeOH/DCM. The impure compound was purified by prep HPLC on an X-Select C18 (19×150) mm 5μ column using a gradient of 10 mM NH₄OAc in H₂O: MeCN as mobile phase, which gave the title compound (33 mg, 23%) as a solid. LCMS (ES+) m/z 479.46 [M+H]⁺. [0911] ¹H NMR (500 MHz, DMSO): δ 8.18 (s, 1H), 7.50 (q, J=3.0 Hz, 1H), 7.43 (q, J=2.9 Hz, 1H), 7.38 (m, J=3.3 Hz, 1H), 7.24 (m, J=3.2 Hz, 1H), 6.93 (s, 1H), 6.41 (d, J=65.7 Hz, 1H), 3.81 (d, J=5.7 Hz, 2H), 3.53 (t, J=6.8 Hz, 1H), 3.38 (s, 3H), 3.35 (s, 3H), 3.18 (d, J=12.4 Hz, 2H), 2.61 (t, J=11.7 Hz, 2H), 2.08 (d, J=6.0 Hz, 1H), 1.64 (d, J=11.1 Hz, 2H), 1.42 (m, J=6.1 Hz, 2H), 1.18 (d, J=6.9 Hz, 6H).

### Example A-4

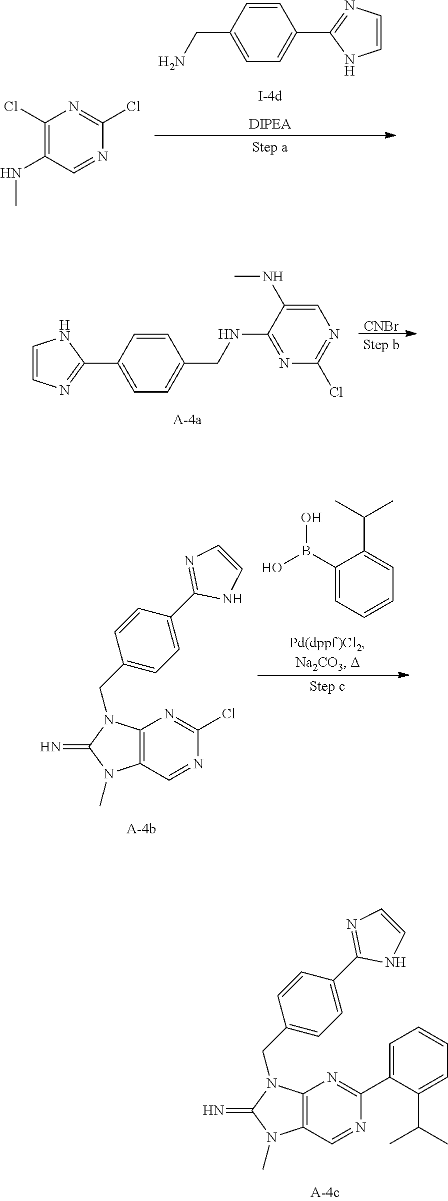

### Step a) N₄-(4-(1H-imidazol-2-yl)benzyl)-2-chloro-N5-methylpyrimidine-4,5-diamine (A-4a)

DIPEA (1.33 mL, 7.70 mmol) was added to a stirred solution of 2,4-dichloro-N-methylpyrimidin-5-amine (354 mg, 1.91 mmol) and compound I-4d (500 mg, 1.91 mmol) in DMF (15 mL) at rt. The resulting mixture was stirred for 4 h at 80° C. Ice cold water (40 mL) was added and the mixture was extracted with EtOAc (3×25 mL). The organic layer was washed with water, brine, dried (Na₂SO₄), filtered and concentrated. The crude compound was triturated with 3% MeOH in diethyl ether. The afforded residue was purified by column chromatography on neutral alumina, eluted with 3-5% MeOH/DCM which gave the title compound (300 mg, 46%) as a solid. LCMS (ES+) 315.22 [M+H]⁺.

### Step b) 9-(4-(1H-imidazol-2-yl)benzyl)-2-chloro-7-methyl-7H-purin-8(9H)-imine (A-4b)

Cyanogen bromide (310 mg, 2.92 mmol) was added at 0° C. to a stirred solution of compound A-4a (250 mg, 0.73 mmol) in EtOH (25 mL). The resulting mixture was stirred at 80° C. for 6 h, then concentrated under reduced pressure, which gave the crude title compound (350 mg) as a liquid. LCMS (ES+) 340.26 [M+H]⁺. The compound was taken to next step without further purification.

### Step c) 9-(4-(1H-imidazol-2-yl)benzyl)-2-(2-isopropylphenyl)-7-methyl-7H-purin-8(9H)-imine (A-4c)

Sodium carbonate (142 mg, 1.34 mmol) and (2-isopropylphenyl) boronic acid (126 mg, 0.80 mmol) were added to a stirred solution of compound A-4b (350 mg, 0.40 mmol) in 1,4-dioxane (6 mL) and water (2 mL) in a sealed tube. The reaction mixture was degassed by bubbling with argon for 10 minutes then Pd(dppf)Cl₂·DCM, (28 mg, 0.04 mmol) was added and the reaction mixture was stirred at 110° C. for 16 h in a sealed tube. The reaction mixture was diluted with ice cold water (10 mL), extracted with EtOAc (3×25 mL) and the combined organic layers were dried (Na₂SO₄) and concentrated under reduced pressure. The afforded crude compound was purified by prep HPLC Sunfire C₁₈ column (30×150) mm 5p using a gradient of 10 mM NH₄HCO₃ in H₂O: MeCN as mobile phase, which gave the title compound (42 mg, 26%) as a solid. LCMS (ES+) m/z 424.50 [M+H]⁺. ¹H NMR (500 MHz, DMSO) δ 12.46 (s, 1H), 8.22 (s, 1H), 7.87 (d, J=8.2 Hz, 2H), 7.47 (q, J=3.0 Hz, 1H), 7.38 (m, J=3.0 Hz, 4H), 7.22 (m, J=4.1 Hz, 2H), 6.99 (s, 1H), 6.53 (d, J=19.0 Hz, 1H), 5.11 (s, 2H), 3.45 (t, J=6.9 Hz, 1H), 3.38 (s, 3H), 1.08 (d, J=6.9 Hz, 6H).

### Example A-5

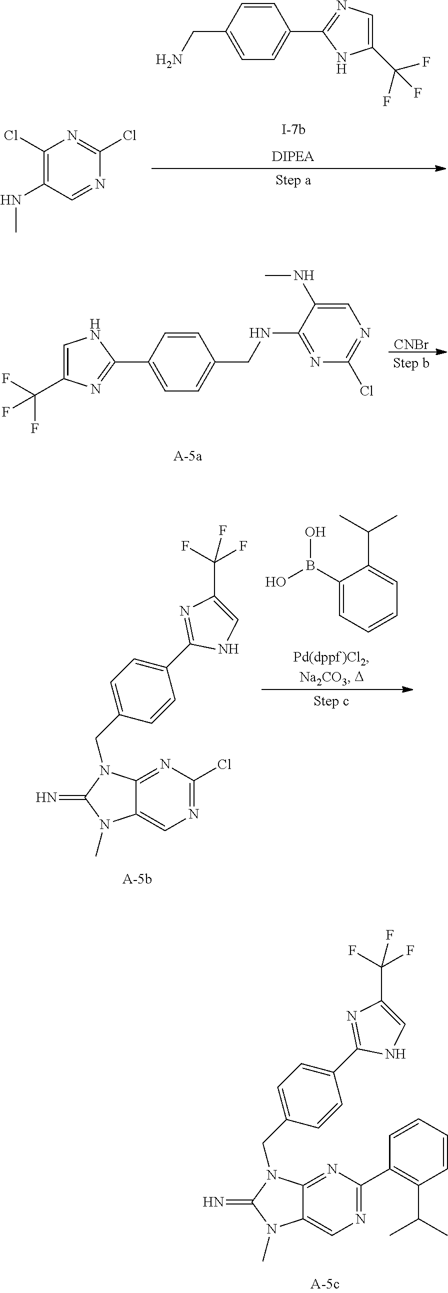

### Step a) 2-chloro-N5-methyl-N4-(4-(4-(trifluoromethyl)-1H-imidazol-2-yl)benzyl)pyrimidine-4,5-diamine (A-5a)

DIPEA (2.4 mL, 13.6 mmol) was added to a stirred solution of compound I-7b (600 mg, 2 mmol) in DMF (10 mL) at rt, 2,4-dichloro-N-methylpyrimidin-5-amine (482 mg, 2.7 mmol) was added and the resulting mixture was stirred for 20 h at 90° C. EtOAc was added and the mixture was washed with water. The organic layer was washed with brine, dried (Na₂SO₄), filtered and concentrated under reduced pressure. The afforded residue was purified by column chromatography on silica gel, eluted with 5-10% MeOH/DCM which gave the title compound (500 mg, 49%) as a solid. LCMS (ES+) 383.36 [M+H]⁺.

### Step b) 2-chloro-7-methyl-9-(4-(4-(trifluoromethyl)-1H-imidazol-2-yl)benzyl)-7,9-dihydro-8H-purin-8-imine (A-5b)

Cyanogen bromide (432 mg, 4.1 mmol) was added at rt. to a stirred solution of compound A-5a (400 mg, 0.8 mmol) in EtOH (20 mL). The resulting mixture was stirred at 80° C. for 16 h, then concentrated under reduced pressure, which gave the crude title compound (500 mg) as a semi-solid. LCMS (ES+) 408.35 [M+H]⁺. The compound was taken to next step without further purification.

### Step c) 2-(2-isopropylphenyl)-7-methyl-9-(4-(4-(trifluoromethyl)-1H-imidazol-2-yl)benzyl)-7,9-dihydro-8H-purin-8-imine (A-5c)

Sodium carbonate (137 mg, 1.3 mmol) was added to a stirred solution of compound A-5b (500 mg, 0.3 mmol) and (2-isopropylphenyl) boronic acid (212 mg, 1.3 mmol) in 1,4-dioxane (6 mL) and water (3 mL). The reaction mixture was degassed by bubbling with argon for 10 minutes then Pd(dppf)Cl₂·DCM, (105 mg, 0.13 mmol) was added and the reaction mixture was stirred at 100° C. for 2 h in a microwave. The reaction mixture was diluted with water and filtered through a celite bed, the filtrate was extracted with EtOAc and the combined organic layers were washed with brine, dried (Na₂SO₄) and concentrated under reduced pressure. The afforded crude compound was combined with another batch and purified by column chromatography on silica gel, eluted with 5-10% MeOH/DCM. The residue was further purified by prep HPLC on an YMC Trait C18 (25×150) mm 10μ column using a gradient of 10 mM NH₄HCO₃ in H₂O: MeCN as mobile phase. The title compound was further purified by prep HPLC on an X-Select C₁₈ (19×150) mm 5μ column using 0.1% formic acid in H₂O: MeCN as mobile phase which gave the title compound (33 mg, 26%) as a solid. LCMS (ES+) m/z 492.51 [M+H]⁺. [0918] ¹H NMR (500 MHz, DMSO): δ 13.16 (s, 1H), 8.22 (s, 1H), 7.91 (d, J=8.4 Hz, 3H), 7.46 (m, J=4.1 Hz, 3H), 7.37 (m, J=3.3 Hz, 2H), 7.22 (m, J=3.2 Hz, 1H), 6.52 (s, 1H), 5.13 (s, 2H), 3.43 (q, J=6.9 Hz, 1H), 3.38 (s, 3H), 1.08 (d, J=6.9 Hz, 6H).

### Example A-6

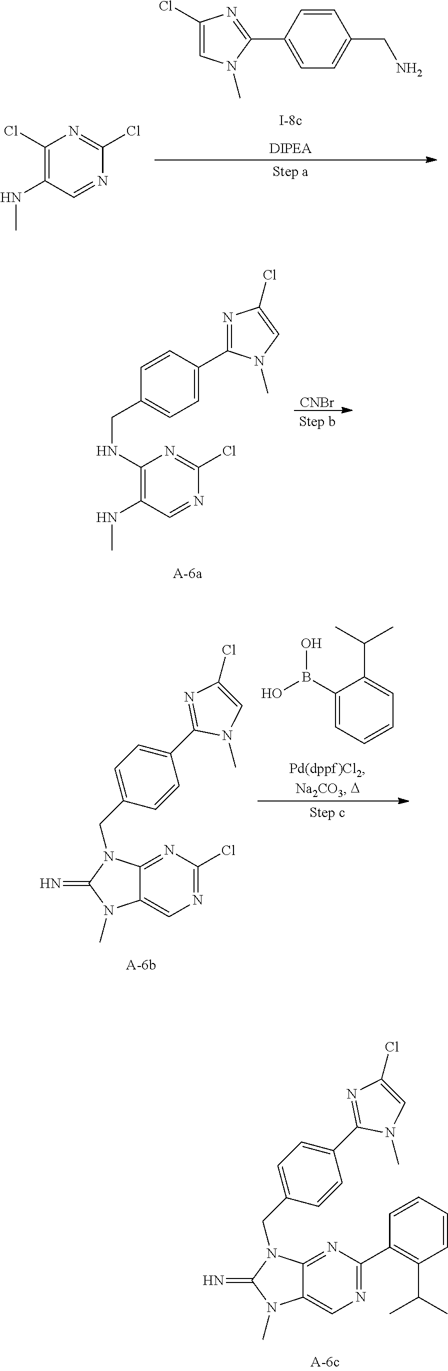

### Step a) 2-chloro-N4-(4-(4-chloro-1-methyl-1H-imidazol-2-yl)benzyl)-N5-methylpyrimidine-4,5-diamine (A-6a)

DIPEA (3.1 mL, 17.8 mmol) was added to a stirred solution of compound I-8c (700 mg, 3.0 mmol) in DMF (10 mL) at rt. 2,4-dichloro-N-methylpyrimidin-5-amine (694 mg, 3.9 mmol) was added and the resulting mixture was stirred for 16 h at 90° C. EtOAc was added and the mixture was washed with water. The organic layer was washed with brine, dried (Na₂SO₄), filtered and concentrated under reduced pressure. The afforded residue was purified by column chromatography on silica gel, eluted with 5-10% MeOH/DCM which gave the title compound (700 mg, 60%) as a solid. LCMS (ES+) 363.38 [M+H]⁺.

### Step b) 2-chloro-9-(4-(4-chloro-1-methyl-1H-imidazol-2-yl)benzyl)-7-methyl-7,9-dihydro-8H-purin-8-imine (A-6b)

Cyanogen bromide (671 mg, 6.3 mmol) was added at rt to a stirred solution of compound A-6a (500 mg, 1.3 mmol) in EtOH (10 mL). The resulting mixture was stirred at 80° C. for 16 h, then concentrated under reduced pressure, which gave the crude title compound (490 mg) as a semi-solid. The compound was taken to next step without further purification.

### Step c) 9-(4-(4-chloro-1-methyl-1H-imidazol-2-yl)benzyl)-2-(2-isopropylphenyl)-7-methyl-7,9-dihydro-8H-purin-8-imine (A-6c)

Sodium carbonate (683 mg, 6.4 mmol) was added to a stirred solution of compound A-6b (500 mg, 1.3 mmol) and (2-isopropylphenyl) boronic acid (1.1 g, 6.4 mmol) in 1,4-dioxane (6 mL) and water (2 mL). The reaction mixture was degassed by bubbling with argon for 10 minutes then Pd(dppf)Cl₂·DCM, (526 mg, 0.6 mmol) was added and the reaction mixture was stirred at 100° C. for 16 h in a sealed tube. The reaction mixture was diluted with water and filtered through the celite bed, the filtrate was extracted with EtOAc and the combined organic layers were washed with brine, dried (Na₂SO₄) and concentrated under reduced pressure. The afforded crude compound was combined with another batch and purified by column chromatography on silica gel, eluted with 5-10% MeOH/DCM. The residue was further purified by prep HPLC on an YMC Trait C18 (25×150) mm 10μ column using a gradient of 10 mM NH₄HCO₃ in H₂O: MeCN as mobile phase. The title compound was further purified by prep HPLC on an X-Select C₁₈ (19×150) mm 5μ column using 0.1% formic acid in H₂O: MeCN as mobile phase which gave the title compound (63 mg, 10%) as a solid. LCMS (ES+) m/z 472.48 [M+H]⁺.

¹H NMR (500 MHz, DMSO): δ 8.22 (s, 1H), 7.62 (d, J=8.1 Hz, 2H), 7.46 (m, J=4.9 Hz, 3H), 7.37 (m, J=4.1 Hz, 3H), 7.22 (m, J=3.2 Hz, 1H), 6.51 (t, J=16.2 Hz, 1H), 5.15 (d, J=32.5 Hz, 2H), 3.44 (t, J=6.8 Hz, 3H), 3.38 (s, 3H), 1.08 (d, J=6.9 Hz, 6H).

### Example 4-7

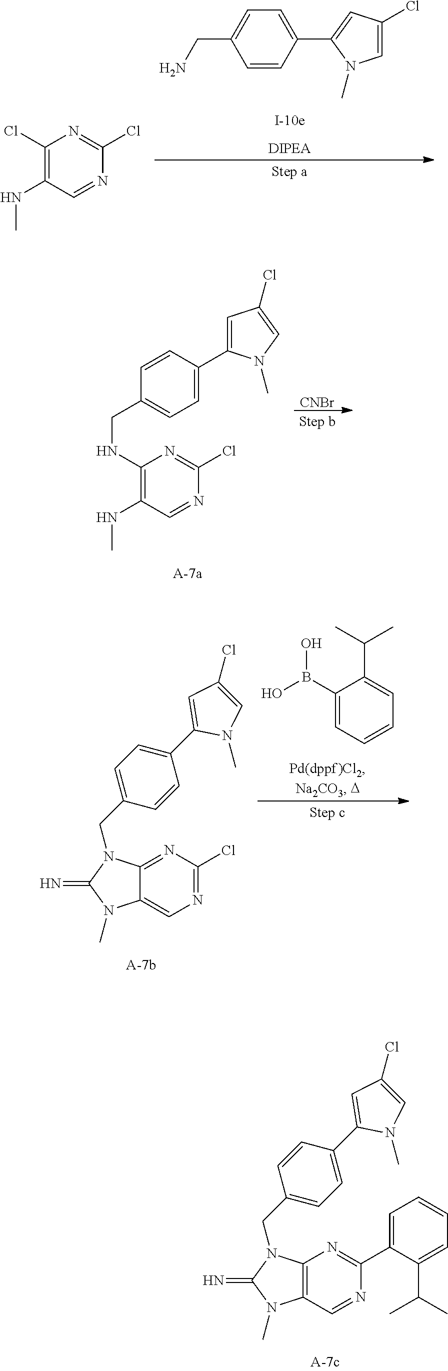

### Step a) 2-chloro-N4-(4-(4-chloro-1-methyl-1H-pyrrol-2-yl)benzyl)-N5-methylpyrimidine-4,5-diamine (A-7a)

2,4-dichloro-N-methylpyrimidin-5-amine (622 mg, 3.5 mmol) was added to a stirred solution of compound I-10e (700 mg, 3.2 mmol) in DMF (25 mL) at rt, then potassium carbonate (1.3 g, 9.5 mmol) was added and the resulting mixture was stirred for 6 h at 90° C. Water was added and the mixture was extracted with EtOAc. The organic layer was washed with brine, dried (Na₂SO₄), filtered and concentrated under reduced pressure. The afforded residue was purified by column chromatography on silica gel, eluted with 50-65% EtOAc in pet ether, which gave the title compound (400 mg, 18%) as a solid. LCMS (ES+) 362.20 [M+H]⁺.

### Step b) 2-chloro-9-(4-(4-chloro-1-methyl-1H-pyrrol-2-yl)benzyl)-7-methyl-7,9-dihydro-8H-purin-8-imine (A-7b)

Cyanogen bromide (422 mg, 4.0 mmol) was added at rt to a stirred solution of compound A-7a (400 mg, 1.0 mmol) in EtOH (40 mL). The resulting mixture was stirred at 80° C. for 8 h, then concentrated under reduced pressure. The afforded residue was purified by column chromatography on silica gel, eluted with 3-6% MeOH in DCM, which gave the title compound (280 mg, 22%) as a solid. LCMS (ES+) 387.19 [M+H]⁺.

### Step c) 9-(4-(4-chloro-1-methyl-1H-pyrrol-2-yl)benzyl)-2-(2-isopropylphenyl)-7-methyl-7,9-dihydro-8H-purin-8-imine (A-7c)

(2-isopropylphenyl) boronic acid (44 mg, 0.3 mmol)) was added to a stirred solution of compound A-7b (280 mg, 0.3 mmol) and sodium carbonate (95 mg, 0.9 mmol) in 1,4-dioxane (15 mL) and water (3 mL). The reaction mixture was degassed by bubbling with argon for 10 minutes then Pd(dppf)Cl₂·DCM, (19 mg, 0.02 mmol) was added and the reaction mixture was stirred at 120° C. for 2 h in microwave. The reaction mixture was concentrated under reduced pressure. The afforded crude compound was purified by column chromatography on silica gel, eluted with 3-6% MeOH/DCM. The residue was further purified by prep HPLC on Kromosil C₁₈ (25×150) mm 10μ column using a gradient of 10 mM NH₄HCO₃ in H₂O: MeCN as mobile phase. The title compound was further purified by prep SFC, which gave the title compound (20 mg, 19%) as a solid. LCMS (ES+) m/z 471.47 [M+H]⁺.

¹H NMR (500 MHz, DMSO): δ 8.22 (d, J=25.5 Hz, 1H), 7.49 (q, J=2.9 Hz, 1H), 7.38 (m, J=3.5 Hz, 6H), 7.23 (m, J=3.2 Hz, 1H), 6.96 (d, J=1.9 Hz, 1H), 6.49 (d, J=35.2 Hz, 1H), 6.14 (s, 1H), 5.12 (d, J=47.0 Hz, 2H), 3.56 (s, 3H), 3.44 (d, J=6.6 Hz, 1H), 3.38 (d, J=14.9 Hz, 3H), 1.09 (d, J=6.8 Hz, 6H).

Preparative SFC Conditions
Column/dimensions: Chiralcel AD-H (250×4.6 mm), 5p
CO₂: 60.0%
Co solvent: 40.0% (30 mM methanolic ammonia in methanol)
Total flow: 60.0 g/min
Back pressure: 90.0 bar
UV: 214 nm
Stack time: 6 min
Load/Inj.: 6.0 mg

### Example A-8

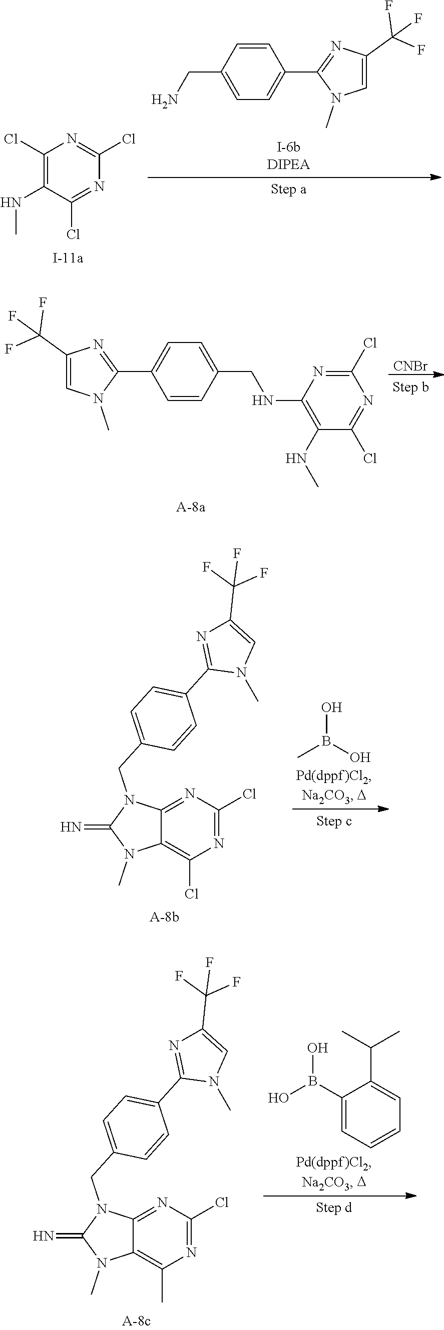

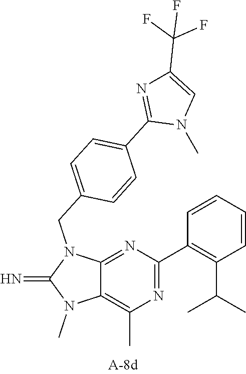 [SMILES: Cc1nc(-c2ccccc2C(C)C)nc2c1n(C)c(=N)n2Cc1ccc(-c2nc(C(F)(F)F)cn2C)cc1]

### Step a) 2,6-dichloro-N5-methyl-N4-(4-(1-methyl-4-(trifluoromethyl)-1H-imidazol-2-yl)benzyl)pyrimidine-4,5-diamine (A-8a)

DIPEA (1.3 mL, 7.5 mmol) was added to a stirred solution of compound I-6b (865 mg, 3.4 mmol) in THE (50 mL) at rt, then compound I-11a (800 mg, 3.8 mmol) was added at 0° C. and the resulting mixture was stirred for 16 h at 80° C. Water (100 mL) was added and the mixture was extracted with EtOAc (3×70 mL). The organic layer was washed with brine (100 mL), dried (Na₂SO₄), filtered and concentrated under reduced pressure. The afforded residue was purified by column chromatography on silica gel, eluted with 25% EtOAc in pet ether, which gave the title compound (1 g, 53%) as a solid. LCMS (ES+) 431.21 [M+H]⁺.

### Step b) 2,6-dichloro-7-methyl-9-(4-(1-methyl-4-(trifluoromethyl)-1H-imidazol-2-yl)benzyl)-7,9-dihydro-8H-purin-8-imine (A-8b)

Cyanogen bromide (533 mg, 5.0 mmol) was added at 0° C. to a stirred solution of compound A-8b (1 g, 2.0 mmol) in EtOH (20 mL). The residue was basified with saturated NaHCO₃ solution. The aqueous layer was extracted with EtOAc (3×70 mL). The combined organic layers were washed with brine, dried (Na₂SO₄), filtered and concentrated under reduced pressure. The afforded residue was purified by column chromatography on silica gel, eluted with 60% EtOAc in pet ether, which gave the crude title compound (600 mg, 64%) as a solid. LCMS (ES+) 456.33 [M+H]⁺.

### Step c) 2-chloro-6,7-dimethyl-9-(4-(1-methyl-4-(trifluoromethyl)-1H-imidazol-2-yl)benzyl)-7,9-dihydro-8H-purin-8-imine (A-8c)

Methyl boronic acid (156 mg, 2.6 mmol) were added to a stirred solution of compound A-8b (600 mg, 1.3 mmol) and sodium carbonate (690 mg, 6.5 mmol) in 1,4-dioxane (16 mL) and water (4 mL). The reaction mixture was degassed by bubbling with argon for 10 minutes then Pd(dppf)Cl₂·DCM, (96 mg, 0.13 mmol) was added and the reaction mixture was stirred at 100° C. for 1 h in microwave. The reaction mixture was diluted with water (100 mL), extracted with EtOAc (3×100 mL). The combined organic layers were washed with water (100 mL), brine (100 mL), dried (Na₂SO₄), filtered and concentrated under reduced pressure. The afforded residue was purified by column chromatography on silica gel, eluted with 6% MeOH in DCM, which gave the title compound (430 g, 64%) as a solid. LCMS (ES+) 436.18 [M+H]⁺.

### Step d) 2-(2-isopropylphenyl)-6,7-dimethyl-9-(4-(1-methyl-4-(trifluoromethyl)-1H-imidazol-2-yl)benzyl)-7,9-dihydro-8H-purin-8-imine (A-8d)

(2-isopropylphenyl) boronic acid (253 mg, 1.5 mmol) was added to a stirred solution of compound A-8c (400 mg, 0.8 mmol) and sodium carbonate (409 mg, 3.9 mmol) in 1,4-dioxane (16 mL) and water (4 mL). The reaction mixture was degassed by bubbling with argon for 10 minutes then Pd(dppf)Cl₂·DCM, (63 mg, 0.08 mmol) was added and the reaction mixture was stirred at 100° C. for 1 h in microwave. The reaction mixture was diluted with water (50 mL) and was extracted with EtOAc (3×50 mL) and the combined organic layers were washed with water (100 mL), brine (100 mL), dried (Na₂SO₄) and concentrated under reduced pressure. The afforded crude compound was combined with another batch and purified by column chromatography on silica gel, eluted with 3% MeOH/DCM. The residue was further purified twice by prep HPLC on Kromosil C18 (25×150) mm 10μ column using a gradient of 10 mM NH₄HCO₃ in H₂O: MeCN as mobile phase, which gave the title compound (143 mg, 36%) as a solid. LCMS (ES+) 520.53 [M+H]⁺.

¹H NMR (500 MHz, DMSO): δ 7.91 (d, J=0.9 Hz, 1H), 7.67 (d, J=8.3 Hz, 2H), 7.47 (m, J=4.6 Hz, 3H), 7.37 (m, J=3.6 Hz, 2H), 7.22 (m, J=3.2 Hz, 1H), 6.51 (s, 1H), 5.17 (s, 2H), 3.74 (s, 3H), 3.57 (s, 3H), 3.42 (m, J=6.9 Hz, 1H), 2.67 (s, 3H), 1.09 (d, J=6.9 Hz, 6H).

### Example A-9c & A-9d

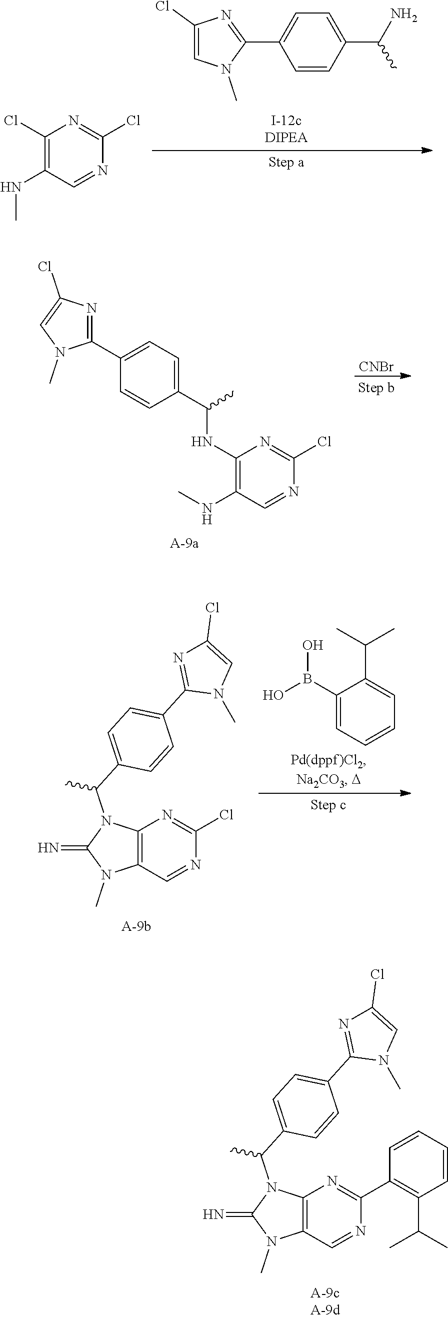

### Step a) 2-chloro-N4-(1-(4-(4-chloro-1-methyl-1H-imidazol-2-yl)phenyl)ethyl)-N₅-methylpyrimidine-4,5-diamine (A-9a)

DIPEA (13.1 mL, 75 mmol) was added to a stirred solution of compound I-12c (2 g, 8.0 mmol) in DMF (25 mL) at rt. 2,4-dichloro-N-methylpyrimidin-5-amine (1.8 g, 10 mmol) was added and the resulting mixture was stirred for 48 h at 90° C. EtOAc (25 mL) was added and the mixture was washed with water (10 mL). The organic layer was washed with brine (10 mL), dried (Na₂SO₄), filtered and concentrated under reduced pressure. The afforded residue was purified by column chromatography on silica gel, eluted with 85% EtOAc/pet ether, which gave the title compound (2.4 g, 81%) as a solid. LCMS (ES+) 377.23 [M+H]⁺.

### Step b) 2-chloro-9-(1-(4-(4-chloro-1-methyl-1H-imidazol-2-yl)phenyl)ethyl)-7-methyl-7,9-dihydro-8H-purin-8-imine (A-9b)

Cyanogen bromide (751 mg, 7.1 mmol) was added at 0° C. to a stirred solution of compound A-9a (700 mg, 1.8 mmol) in EtOH (20 mL). The resulting mixture was stirred at 80° C. for 5 h, then concentrated under reduced pressure. The afforded residue, was cooled to rt and precipitated solid was filtered and vacuum dried, which gave the title compound (200 mg, 28%) as a solid. LCMS (ES+) 402.28 [M+H]⁺.

### Step c) 9-(1-(4-(4-chloro-1-methyl-1H-imidazol-2-yl)phenyl)ethyl)-2-(2-isopropylphenyl)-7-methyl-7,9-dihydro-8H-purin-8-imine (A-9c & A-9d)

To a stirred and degassed solution of compound A-9b (200 mg, 0.5 mmol), (2-isopropylphenyl) boronic acid (121 mg, 0.74 mmol) and sodium carbonate (156 mg, 1.5 mmol) in 1,4-dioxane (10 mL) and water (3 mL), was added Pd(dppf)Cl₂·DCM, (180 mg, 0.25 mmol) was added and the reaction mixture was stirred at 100° C. for 1 h in microwave. The reaction mixture was diluted with water (20 mL) and was extracted with EtOAc (2×25 mL) and combined the organic layers were washed with brine (10 mL), dried (Na₂SO₄) and concentrated under reduced pressure. The residue was purified by prep HPLC on Kromosil C18 (25×150) mm 10μ column using a gradient of 10 mM NH₄HCO₃ in H₂O: MeCN as mobile phase. The racemate was purified with by Chiral SFC, which gave the title compounds A-9c (42 mg, 17%) and A-9d (29 mg, 12%) as a solid. LCMS (ES+) 486.57 [M+H]⁺.

A-9c: ¹H NMR (500 MHz, DMSO): δ 8.20 (s, 1H), 7.62 (d, J=7.6 Hz, 2H), 7.48 (m, J=5.2 Hz, 3H), 7.36 (m, J=3.4 Hz, 3H), 7.21 (m, J=2.7 Hz, 1H), 6.45 (d, J=33.4 Hz, 1H), 5.89 (t, J=16.8 Hz, 1H), 3.68 (s, 3H), 3.46 (q, J=6.8 Hz, 1H), 3.38 (s, 3H), 1.98 (d, J=7.3 Hz, 3H), 1.04 (q, J=5.8 Hz, 6H).

A-9d: ¹H NMR (500 MHz, DMSO): δ 8.20 (s, 1H), 7.62 (d, J=7.5 Hz, 2H), 7.48 (m, J=5.2 Hz, 3H), 7.36 (m, J=3.4 Hz, 3H), 7.21 (m, J=2.7 Hz, 1H), 6.44 (d, J=34.7 Hz, 1H), 5.89 (t, J=18.5 Hz, 1H), 3.68 (s, 3H), 3.47 (m, J=6.8 Hz, 1H), 3.37 (d, J=10.5 Hz, 3H), 1.98 (d, J=7.3 Hz, 3H), 1.04 (q, J=5.9 Hz, 6H).

Preparative SFC Conditions
Column/dimensions: Chiralcel OJ-H (250×4.6 mm), 5p
CO₂: 60.0%
Co-solvent: 40.0% (30 mM methanolic ammonia in ethanol)
Total flow: 70.0 g/min
Back pressure: 100.0 bar
UV: 214 nm
Stack time: 20.1 min
Load/Inj.: 8.8 mg

### Example A-10d & A-10e

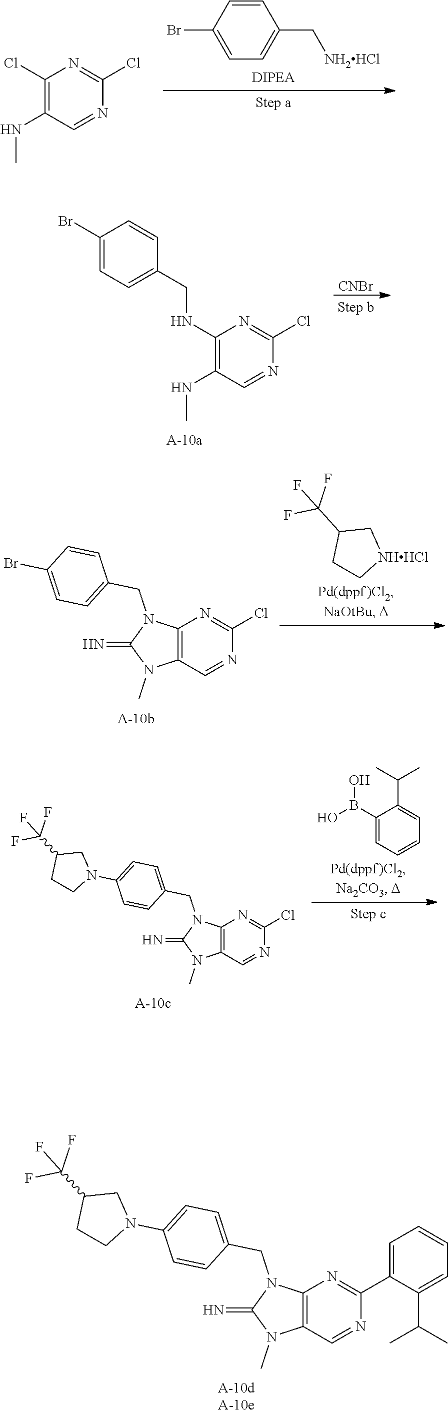

### Step a) N₄-(4-bromobenzyl)-2-chloro-N5-methylpyrimidine-4,5-diamine (A-10a)

DIPEA (13.8 mL, 78.7 mmol) was added to a stirred solution of (4-bromophenyl)methanamine hydrochloride (7 g, 31.7 mmol) in DMF (30 mL) at rt and stirred for 5 min. 2,4-dichloro-N-methylpyrimidin-5-amine (5 g, 28.3 mmol) was added at 0° C. and the resulting mixture was stirred for 16 h at 80° C., then concentrated under reduced pressure. Water was added to the residue and stirred for 30 min. The precipitated solid was filtered and dried, which gave the title compound (4.5 g, 39%) as a solid. LCMS (ES+) 329.12 [M+H]⁺.

### Step b) 9-(4-bromobenzyl)-2-chloro-7-methyl-7,9-dihydro-8H-purin-8-imine (A-10b)

Cyanogen bromide (3.6 g, 34.3 mmol) was added at 0° C. to a stirred solution of compound A-10a (4.5 g, 13.7 mmol) in EtOH (40 mL). The resulting mixture was stirred at 80° C. for 16 h, then cooled to rt. The precipitated solid was filtered and vacuum dried, which gave the title compound (3 g, 58%) as a solid. LCMS (ES+) 354.11 [M+H]⁺.

### Step c) 2-chloro-7-methyl-9-(4-(3-(trifluoromethyl)pyrrolidin-1-yl)benzyl)-7,9-dihydro-8H-purin-8-imine (A-10c)

3-(Trifluoromethyl)pyrrolidine hydrochloride (450 mg, 2.6 mmol) was added to a stirred solution of compound A-10b (1 g, 2.8 mmol) and NaOtBu (1.4 g, 14.2 mmol) in 1,4-dioxane (30 mL). The resulting mixture was purged with argon for 10 minutes followed by addition of 2-dicyclohexylphosphino-2′,4′,6′-triisopropylbiphenyl (270 mg, 0.6 mmol) and Pd(dppf)Cl₂·DCM (232 mg, 0.3 mmol). The mixture was again degassed with argon for 2 minutes and stirred at 60° C. for 16 h. The reaction mixture was diluted with water (80 mL) and extracted with EtOAc (3×80 mL). The combined organic layer was washed with water (100 mL) and brine (100 mL), dried (Na₂SO₄), filtered and concentrated under reduced pressure, which gave the crude title compound (2 g, 47%) as a solid. LCMS (ES+) m/z 411.38 [M+H]⁺. The compound was taken to next step without further purification.

### Step d) 2-(2-isopropylphenyl)-7-methyl-9-(4-(3-(trifluoromethyl)pyrrolidin-1-yl)benzyl)-7,9-dihydro-8H-purin-8-imine (A-10d & A-10e)

(2-isopropylphenyl) boronic acid (112 mg, 0.7 mmol) was added to a stirred solution of compound A-10c (1 g, 0.7 mmol) and sodium carbonate (360 mg, 3.4 mmol) in 1,4-dioxane (10 mL) and water (2.5 mL). The reaction mixture was degassed by bubbling with argon for 5 minutes, then Pd(dppf)Cl₂·DCM, (56 mg, 0.07 mmol) was added and the reaction mixture was stirred at 100° C. for 2 h in microwave. The reaction mixture was diluted with water (50 mL) and was extracted with EtOAc (3×50 mL) and the combined organic layers were washed with water (80 mL), brine (80 mL), dried (Na₂SO₄) and concentrated under reduced pressure. The afforded crude compound was purified by column chromatography on neutral alumina, eluted with 40% EtOAc/pet ether. The residue was further purified by prep HPLC on Kromosil C18 (25×150) mm 10μ column using a gradient of 10 mM NH₄HCO₃ in H₂O: MeCN as mobile phase. The racemate was purified by Chiral SFC.

### A-10d

The compound obtained from the first fraction was further purified by prep HPLC on Kromosil C18 (25×150) mm 10μ column using a gradient of 10 mM NH₄HCO₃ in H2O: MeCN as mobile phase and lyophilised, which gave the title compound (12.9 mg, 3%) as a solid. MS (ES+) m/z 495.42 [M+H]⁺.

¹H NMR (500 MHz, DMSO): δ 8.18 (d, J=28.1 Hz, 1H), 7.50 (d, J=7.6 Hz, 1H), 7.39 (m, J=3.9 Hz, 2H), 7.25 (m, J=4.9 Hz, 3H), 6.54 (d, J=6.7 Hz, 2H), 6.38 (s, 1H), 4.96 (d, J=36.5 Hz, 2H), 3.49 (m, J=7.3 Hz, 2H), 3.35 (s, 3H), 3.25 (m, J=6.1 Hz, 2H), 2.24 (m, J=3.7 Hz, 1H), 2.05 (m, J=7.0 Hz, 1H), 1.14 (d, J=6.9 Hz, 6H).

### A-10e

The compound obtained from the second fraction was further purified by chiral SFC. The impure compound was further purified by prep HPLC on Kromosil C18 (25×150) mm 10p column using a gradient of 10 mM NH₄HCO₃ in H₂O: MeCN as mobile phase, which gave the title compound (13 mg, 3%) as a solid. MS (ES+) m/z 495.42 [M+H]⁺.

¹H NMR (500 MHz, DMSO): δ 8.18 (s, 1H), 7.50 (q, J=3.0 Hz, 1H), 7.39 (m, J=3.9 Hz, 2H), 7.24 (m, J=4.1 Hz, 3H), 6.54 (d, J=8.7 Hz, 2H), 6.43 (s, 1H), 4.96 (s, 2H), 3.49 (m, J=7.2 Hz, 2H), 3.34 (s, 3H), 3.25 (m, J=6.1 Hz, 2H), 2.25 (m, J=3.8 Hz, 1H), 2.05 (m, J=7.0 Hz, 1H), 1.14 (d, J=6.9 Hz, 6H).

Conditions for Chiral SFC
Column/dimensions: Chiralpak IC (30×250) mm, 5μ
% CO₂: 60.0%
% Co solvent: 40.0% (0.5% isopropyl amine in isopropanol)
Total Flow: 70.0 g/min
Back Pressure: 90.0 bar
UV: 214 nm
Stack time: 10.6 min
Load/Inj: 4.5 mg
Conditions for chiral SFC for A-10e
Column/dimensions: Chiralpak IC (30×250) mm, 5p
% CO₂: 65.0%
% Co solvent: ³5.0% (0.5% isopropyl amine in isopropanol)
Total Flow: 100.0 g/min
Back Pressure: 100.0 bar
UV: 214 nm
Stack time: 8.5 min
Load/Inj: 3.0 mg

### Example A-11

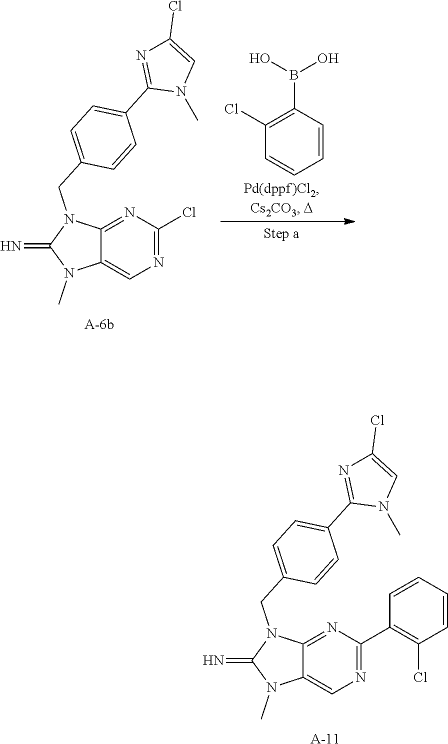

### Step a) 9-(4-(4-chloro-1-methyl-1H-imidazol-2-yl)benzyl)-2-(2-chlorophenyl)-7-methyl-7,9-dihydro-8H-purin-8-imine (A-11)

A stirred solution of compound A-6b (50 mg, 0.11 mmol), (2-chlorophenyl)boronic acid (21 mg, 0.13 mmol) and cesium carbonate (108 mg, 0.33 mmol) in 1,4-dioxane (2 mL) in a microwave vial was degassed by bubbling with argon for 15 minutes, then Pd(dppf)Cl₂·DCM, (8 mg, 0.01 mmol) was added and the reaction mixture was again degassed by bubbling with argon for 5 minutes, then stirred at 100° C. for 2 h in microwave. The reaction mixture was diluted with EtOAc, filtered through the celite bed. The filtrate was concentrated under reduced pressure. The crude was combined with another batch and purified by prep HPLC on Kromosil C18 (25×150) mm 10μ column using a gradient of 10 mM NH₄HCO₃ in H₂O: MeCN as mobile phase and lyophilised, which gave the title compound (18 mg) as a solid. MS (ES+) m/z 464.40 [M+H]⁺. ¹H NMR (500 MHz, DMSO): δ 8.28 (s, 1H), 7.65 (m, J=8.2 Hz, 3H), 7.50 (d, J=8.1 Hz, 3H), 7.43 (t, J=3.7 Hz, 2H), 7.35 (s, 1H), 6.80 (s, 1H), 5.16 (s, 2H), 3.68 (s, 3H), 3.40 (s, 3H).

### Example A-12

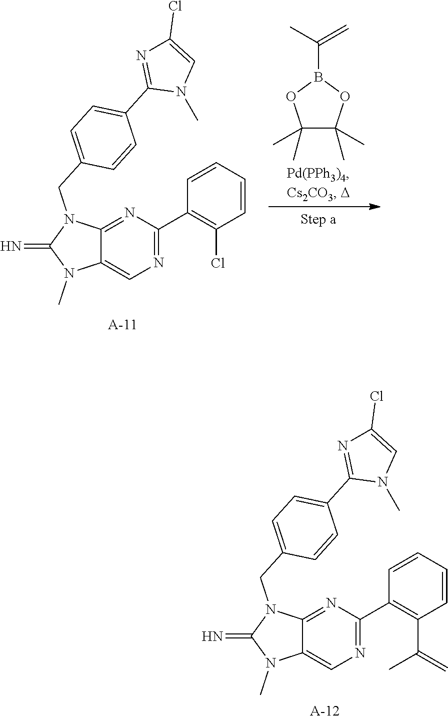

### Step a) 9-(4-(4-chloro-1-methyl-1H-imidazol-2-yl)benzyl)-7-methyl-2-(2-(prop-1-en-2-yl)phenyl)-7,9-dihydro-8H-purin-8-imine (A-12)

A stirred solution of compound A-11 (340 mg, 0.5 mmol), 2-isopropenyl-4,4,5,5-tetramethyl-1,3,2-dioxaborolane (0.13 mL, 0.7 mmol) and cesium carbonate (374 mg, 1.1 mmol) in toluene (6 mL) in a microwave vial was degassed by bubbling with argon for 15 minutes, then Pd(PPh₃)₄ (51 mg, 0.04 mmol) was added, the mixture was degassed for 5 minutes, then stirred at 120° C. for 3 h in a microwave. The reaction mixture was diluted with EtOAc, filtered through the celite bed. The filtrate was concentrated under reduced pressure and triturated with diethyl ether. The obtained crude was further purified twice by prep HPLC on Kromosil C18 (25×150) mm 10μ column using a gradient of 10 mM NH₄HCO₃ in H₂O: MeCN as mobile phase and lyophilised, which gave the title compound (45 mg, 20%) as a solid. MS (ES+) m/z 470.46 [M+H]⁺.

¹H NMR (500 MHz, DMSO): δ 8.20 (s, 1H), 7.65 (m, J=3.9 Hz, 3H), 7.46 (d, J=8.4 Hz, 2H), 7.36 (m, J=1.9 Hz, 3H), 7.26 (m, J=2.2 Hz, 1H), 6.52 (s, 1H), 5.12 (s, 2H), 4.85 (t, J=1.7 Hz, 1H), 4.61 (d, J=1.2 Hz, 1H), 3.69 (s, 3H), 3.37 (s, 3H), 1.78 (s, 3H).

### Example A-13

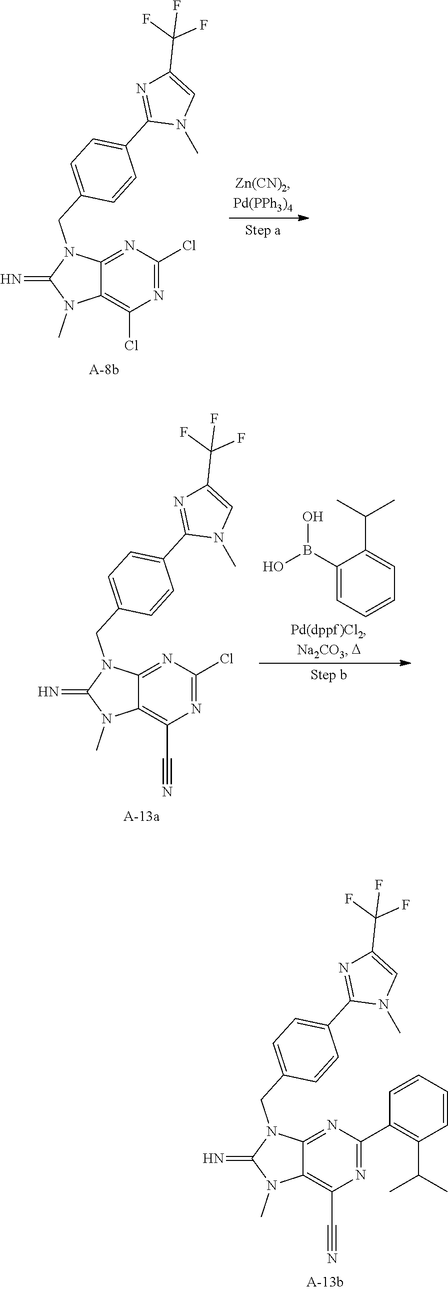

### Step a) 2-chloro-8-imino-7-methyl-9-(4-(1-methyl-4-(trifluoromethyl)-1H-imidazol-2-yl)benzyl)-8,9-dihydro-7H-purine-6-carbonitrile (A-13a)

Zn(CN)₂ (129 mg, 1.1 mmol) was added at rt to a stirred solution of compound A-8b (500 mg, 1.1 mmol) in DMF (20 mL). Reaction mixture was degassed with argon for 5 min, then Pd(PPh₃)₄ (115 mg, 0.11 mmol) was added and the mixture was again degassed with argon for 2 min. The reaction mixture was stirred at 120° C. for 3 h, then concentrated under reduced pressure. The residue was diluted with water and stirred for 5 min. The precipitated solid was filtered and dried. The afforded crude compound was purified by column chromatography on neutral alumina, eluted with 3% MeOH/DCM, which gave the title compound (600 mg, 41%) as a solid. LCMS (ES+) m/z 447.44 [M+H]⁺.

### Step b) 8-imino-2-(2-isopropylphenyl)-7-methyl-9-(4-(1-methyl-4-(trifluoromethyl)-1H-imidazol-2-yl)benzyl)-8,9-dihydro-7H-purine-6-carbonitrile (A-13b)

(2-isopropylphenyl) boronic acid (303 mg, 1.85 mmol) was added to a stirred solution of compound A-13a (550 mg, 1.2 mmol) and sodium carbonate (652 mg, 6.2 mmol) in 1,4-dioxane (12 mL) and water (3 mL). The reaction mixture was degassed by bubbling with argon for 10 minutes, then Pd(dppf)Cl₂·DCM (101 mg, 0.12 mmol) was added and the mixture was again degassed with argon for 2 min. The resulting reaction mixture was stirred at 100° C. for 1 h in microwave. The reaction mixture was diluted with water (50 mL) and was extracted with EtOAc (3×60 mL) and the combined organic layers were washed with water (50 mL), brine (50 mL), dried (Na₂SO₄), filtered and concentrated under reduced pressure. The afforded crude compound was purified by column chromatography on neutral alumina, eluted with 3% MeOH/DCM. The residue was further purified by prep HPLC on Kromosil C18 (25×150) mm 10μ column using a gradient of 10 mM NH₄HCO₃ in H₂O: MeCN as mobile phase. The second fraction was pooled and concentrated under reduced pressure, which gave the title compound (85 mg, 12%) as a solid. LCMS (ES+) m/z 531.55 [M+H]⁺.

¹H NMR (500 MHz, DMSO): δ 7.92 (s, 1H), 7.68 (t, J=7.2 Hz, 2H), 7.44 (m, J=9.1 Hz, 6H), 7.25 (m, J=4.0 Hz, 1H), 5.21 (d, J=60.5 Hz, 2H), 3.75 (s, 3H), 3.58 (d, J=16.2 Hz, 3H), 3.34 (d, J=2.5 Hz, 1H), 1.09 (q, J=5.8 Hz, 6H).

### Example A-14

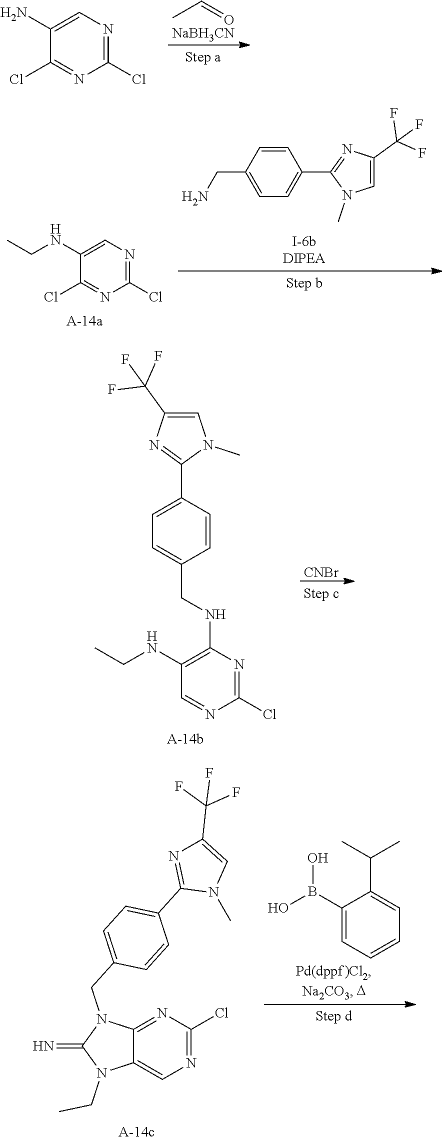

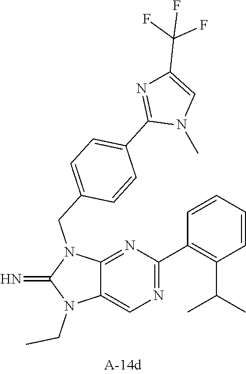 [SMILES: CCn1c(=N)n(Cc2ccc(-c3nc(C(F)(F)F)cn3C)cc2)c2nc(-c3ccccc3C(C)C)ncc21]

### Step a) 2,4-dichloro-N-ethylpyrimidin-5-amine (A-14a)

To a stirred solution of 2,4-dichloropyrimidin-5-amine (1 g, 6 mmol) in MeOH (25 mL) and acetic acid (2 mL) was added acetaldehyde (1.3 g, 30 mmol) followed by NaBH₃CN (1.94 g, 30 mmol) at 0° C. The resultant reaction mixture was stirred at rt for 16 h, then concentrated under reduced pressure. The residue was dissolved in water and extracted with DCM. The combined organic layer was washed with brine, dried (Na₂SO₄), filtered and concentrated under reduced pressure. The crude compound was purified by column chromatography on silica gel and eluted with 25-30% EtOAc/hexane, which gave the title compound (530 mg, 44%) as a liquid.

MS (ES+) 192.09 [M+H]⁺.

### Step b)

### 2-chloro-N₅-ethyl-N4-(4-(1-methyl-4-(trifluoromethyl)-1H-imidazol-2-yl)benzyl)pyrimidine-4,5-diamine (A-14b)

DIPEA (970 mg, 7 mmol) was added followed by addition of 2,4-dichloro-5-nitropyrimidine (480 mg, 2.0 mmol) to a stirred solution compound I-6b (640 mg, 2 mmol) in DMF (5 mL) at 0° C. The resulting mixture was stirred for 16 h at 90° C. Water was added and the mixture was extracted with EtOAc. The organic layer was washed with brine, dried (Na₂SO₄), filtered and concentrated. The crude compound was purified by column chromatography on silica gel and eluted with 55-65% EtOAc/hexane, which gave the title compound (800 mg, 55%) as a liquid. LCMS (ES+) 411.29 [M+H]⁺. The compound was taken to next step without further purification.

### Step c) 2-chloro-7-ethyl-9-(4-(1-methyl-4-(trifluoromethyl)-1H-imidazol-2-yl)benzyl)-7,9-dihydro-8H-purin-8-imine (A-14c)

Cyanogen bromide (700 mg, 6.6 mmol) was added at 0° C. to a stirred solution of compound A-14b (750 mg, 1.6 mmol) in EtOH (25 mL). The resulting mixture was stirred at 90° C. for 16 h, then concentrated under reduced pressure. The crude compound was purified by column chromatography on silica gel and eluted with 4-6% MeOH/DCM, which gave the title compound (750 mg, 29%) as a liquid. LCMS (ES+) 436.25 [M+H]⁺. The compound was taken to next step without further purification.

### Step d) 7-ethyl-2-(2-isopropylphenyl)-9-(4-(1-methyl-4-(trifluoromethyl)-1H-imidazol-2-yl)benzyl)-7,9-dihydro-8H-purin-8-imine (A-14d)

To a stirred and degassed solution of compound A-14c (700 mg, 1.6 mmol) in 1,4-dioxane (12 mL) and water (3 mL) in a microwave vial, were added potassium carbonate (444 mg, 3.2 mmol), Pd(dppf)Cl₂·DCM (131 mg, 0.16 mmol) followed by addition of (2-isopropylphenyl) boronic acid (316 mg, 1.9 mmol) and the mixture was again degassed with argon for 5 min. The resulting reaction mixture was stirred at 120° C. for 2 h in microwave. The reaction mixture was concentrated, diluted with water, extracted with EtOAc. The combined organic layer was washed with brine, dried (Na₂SO₄), filtered and concentrated under reduced pressure. The crude compound was purified by column chromatography on silica gel and eluted with 4-5% MeOH/DCM. The impure compound was purified by prep HPLC on a Kromosil C18 (25×150) mm 10μ column using a gradient of 10 mM NH₄HCO₃ in H₂O: MeCN as mobile phase, which gave the title compound (50 mg, 5%) as a solid. LCMS (ES+) m/z 520.57 [M+H]⁺. ¹H NMR (500 MHz, DMSO): δ 8.28 (d, J=22.4 Hz, 1H), 7.91 (d, J=0.9 Hz, 1H), 7.68 (d, J=7.8 Hz, 2H), 7.48 (q, J=3.0 Hz, 3H), 7.38 (m, J=3.8 Hz, 2H), 7.22 (m, J=3.2 Hz, 1H), 6.56 (s, 1H), 5.17 (d, J=48.1 Hz, 2H), 3.95 (s, 2H), 3.75 (s, 3H), 3.44 (q, J=6.6 Hz, 1H), 1.23 (s, 3H), 1.10 (d, J=6.8 Hz, 6H).

### Example A-15

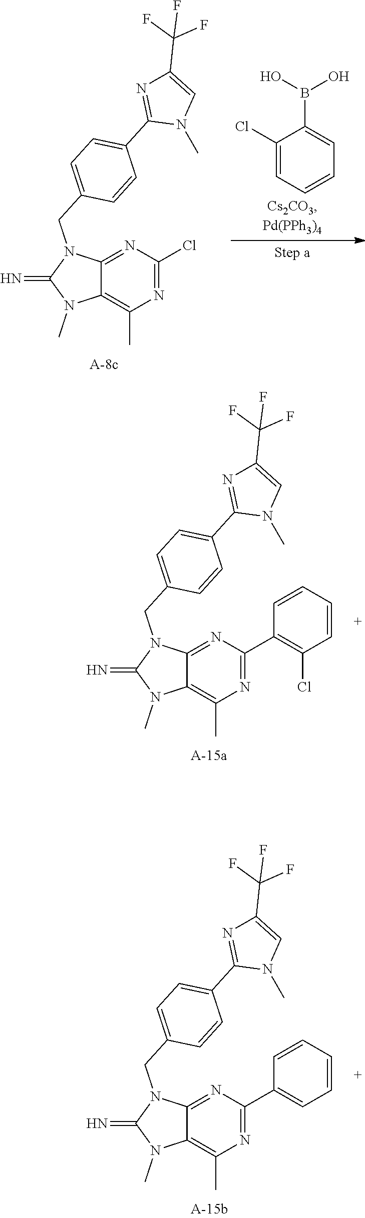 [SMILES: Cc1nc(Cl)nc2c1n(C)c(=N)n2Cc1ccc(-c2nc(C(F)(F)F)cn2C)cc1 ; OB(O)c1ccccc1Cl ; Cc1nc(-c2ccccc2Cl)nc2c1n(C)c(=N)n2Cc1ccc(-c2nc(C(F)(F)F)cn2C)cc1 ; Cc1nc(-c2ccccc2)nc2c1n(C)c(=N)n2Cc1ccc(-c2nc(C(F)(F)F)cn2C)cc1]

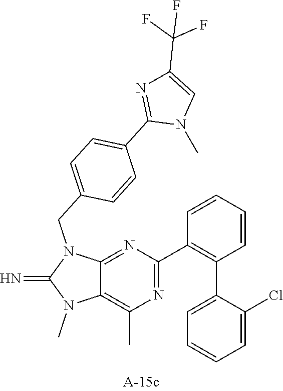 [SMILES: Cc1nc(-c2ccccc2-c2ccccc2Cl)nc2c1n(C)c(=N)n2Cc1ccc(-c2nc(C(F)(F)F)cn2C)cc1]

### Step a) 2-(2-chlorophenyl)-6,7-dimethyl-9-(4-(1-methyl-4-(trifluoromethyl)-1H-imidazol-2-yl)benzyl)-7,9-dihydro-8H-purin-8-imine; 6,7-dimethyl-9-(4-(1-methyl-4-(trifluoromethyl)-1H-imidazol-2-yl)benzyl)-2-phenyl-7,9-dihydro-8H-purin-8-imine & 2-(2′-chloro-[1,1′-biphenyl]-2-yl)-6,7-dimethyl-9-(4-(1-methyl-4-(trifluoromethyl)-1H-imidazol-2-yl)benzyl)-7,9-dihydro-8H-purin-8-imine (A-15a; A-15b & A-15c)

To a stirred solution of compound A-8c (220 mg, 0.42 mmol) in toluene (15 mL) in a microwave vial, were added (2-chlorophenyl)boronic acid (83 mg, 0.53 mmol) and cesium carbonate (415 mg, 1.3 mmol) was degassed by bubbling with argon for 15 minutes, then Pd(PPh₃)₄ (50 mg, 0.04 mmol) was added, the mixture was degassed for 5 minutes, then stirred at 100° C. for 1.5 h in a microwave. The reaction mixture was filtered through the celite bed. The filtrate was concentrated under reduced pressure The crude compound was combined with another batch and purified by column chromatography on silica gel and eluted with 7% MeOH/DCM (two fractions were collected). The residue from the second fraction was further purified by prep HPLC on a Kromosil C18 (25×150) mm 10μ column using a gradient of 10 mM NH₄HCO₃ in H₂O: MeCN as mobile phase, which gave three peaks.

### A-15a

Peak-1 was concentrated and lyophilised, which gave the title compound (80 mg) as a solid. MS (ES+) m/z 512.46 [M+H]⁺.

¹H NMR (500 MHz, DMSO): δ 10.90 (s, 1H), 10.83 (s, 1H), 7.90 (d, J=0.7 Hz, 1H), 7.77 (s, 1H), 7.58 (d, J=8.2 Hz, 2H), 7.38 (m, J=5.2 Hz, 2H), 7.19 (m, J=4.3 Hz, 3H), 6.99 (d, J=7.4 Hz, 1H), 4.26 (t, J=7.2 Hz, 1H), 4.09 (d, J=21.4 Hz, 2H), 3.74 (s, 3H), 2.73 (t, J=6.9 Hz, 1H), 1.06 (d, J=29.2 Hz, 6H).

### A-15b

Peak-2 was concentrated and lyophilised, which gave the title compound (60 mg) as a solid. MS (ES+) m/z 478.45 [M+H]⁺.

¹H NMR (500 MHz, DMSO): δ 8.32 (m, J=1.9 Hz, 2H), 7.90 (d, J=0.9 Hz, 1H), 7.69 (d, J=7.8 Hz, 2H), 7.55 (d, J=6.5 Hz, 2H), 7.44 (m, J=3.8 Hz, 3H), 6.36 (d, J=20.2 Hz, 1H), 5.24 (d, J=49.0 Hz, 2H), 3.74 (s, 3H), 3.55 (s, 3H), 2.69 (s, 3H).

A-15c: Peak-3 was concentrated and lyophilised, which gave the title compound (22 mg) as a solid. MS (ES+) m/z 588.58 [M+H]⁺.

¹H NMR (500 MHz, DMSO); δ 8.10 (d, J=5.0 Hz, 1H), 7.91 (s, 1H), 7.63 (s, 2H), 7.50 (m, J=3.0 Hz, 2H), 7.25 (m, J=6.3 Hz, 6H), 6.17 (t, J=16.4 Hz, 1H), 4.67 (m, J=20.0 Hz, 2H), 3.74 (s, 3H), 3.46 (s, 3H), 2.49 (s, 3H).

### Example A-16

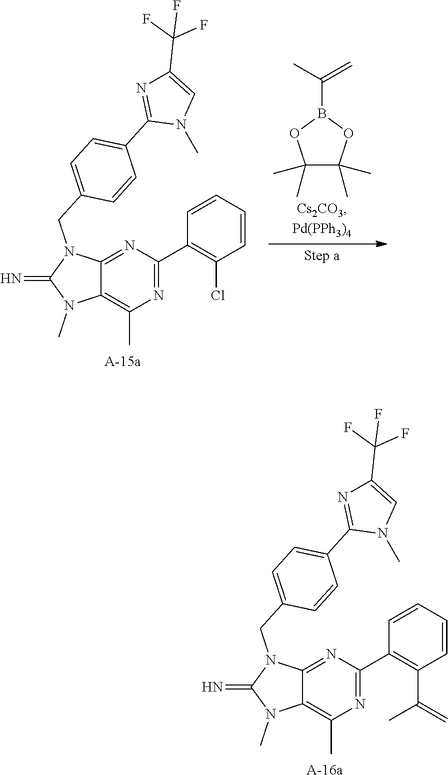

### Step b) 6,7-dimethyl-9-(4-(1-methyl-4-(trifluoromethyl)-1H-imidazol-2-yl)benzyl)-2-(2-(prop-1-en-2-yl)phenyl)-7,9-dihydro-8H-purin-8-imine (A-16a)

To a stirred solution of compound A-15a (250 mg, 0.23 mmol) in toluene (10 mL) in a microwave vial, were added 4,4,5,5-tetramethyl-2-(prop-1-en-2-yl)-1,3,2-dioxaborolane (51 mg, 0.3 mmol) and cesium carbonate (191 mg, 0.6 mmol) was degassed by bubbling with argon for 15 minutes, then Pd(PPh₃)₄ (27 mg, 0.02 mmol) was added, the mixture was degassed for 5 minutes, then stirred at 100° C. for 1.5 h in a microwave. The reaction mixture was filtered through the celite bed and the filtrate was concentrated under reduced pressure. The crude compound was combined with another batch and purified by column chromatography on silica gel and eluted with 6% MeOH/DCM. The impure compound was purified by prep HPLC on a Kromosil C18 (25×150) mm 10μ column using a gradient of 10 mM NH₄HCO₃ in H₂O: MeCN as mobile phase. The residue was further purified twice with SFC. The impure residue was further purified by prep HPLC on a Kromosil C18 (25×150) mm 10μ column using a gradient of 10 mM NH₄HCO₃ in H₂O: MeCN as mobile phase and lyophilised, which gave the title compound (50 mg) as a solid. MS (ES+) m/z 518.59 [M+H]⁺.

¹H NMR (500 MHz, DMSO): δ 7.92 (d, J=0.8 Hz, 1H), 7.68 (m, J=2.1 Hz, 3H), 7.47 (d, J=8.1 Hz, 2H), 7.35 (m, J=2.8 Hz, 2H), 7.24 (m, J=2.2 Hz, 1H), 6.30 (s, 1H), 5.11 (s, 2H), 4.84 (s, 1H), 4.59 (s, 1H), 3.75 (s, 3H), 3.54 (s, 3H), 2.66 (s, 3H), 1.81 (s, 3H).

Conditions for Preparative SFC-1
Column/dimensions: Chiralpak IG (30×250) mm, 5μ
% CO₂: 70.0%
% Co solvent: 30.0% (0.5% diethyl amine in methanol)
Total Flow: 70.0 g/min
Back Pressure: 90.0 bar
UV: 214 nm
Stack time: 5.6 min
Load/Inj: 5.5 mg
Conditions for preparative SFC
Column/dimensions: Chiralpak IG (30×250) mm, 5p
% CO₂: 55.0%
% Co solvent: ⁴5.0% (0.5% diethyl amine in methanol)
Total Flow: 70.0 g/min
Back Pressure: 100.0 bar
UV: 214 nm
Stack time: 4.0 min
Load/Inj: 12.5 mg

### Example A-17

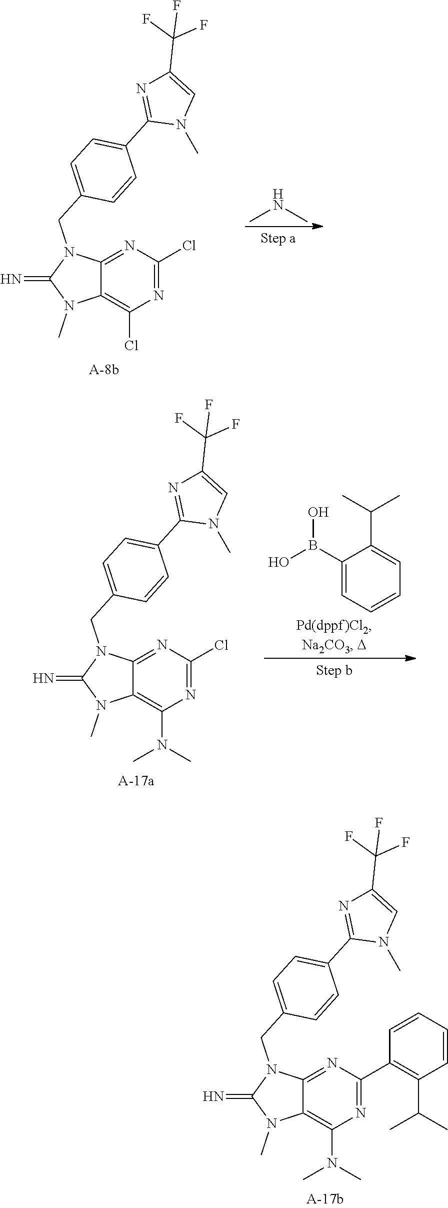

### Step b) 2-chloro-8-imino-N,N, 7-trimethyl-9-(4-(1-methyl-4-(trifluoromethyl)-1H-imidazol-2-yl)benzyl)-8,9-dihydro-7H-purin-6-amine (A-17a)

A solution of compound A-8b (300 mg, 0.7 mmol) in dimethylamine (2M in MeOH) (3.3 mL, 6.6 mmol) in a sealed tube was stirred at rt for 16 h, then concentrated under reduced pressure, which gave the title compound (270 mg, 66%) as a solid. MS (ES+) m/z 465.35 [M+H]⁺.

### Step b) 8-imino-2-(2-isopropylphenyl)-N,N,7-trimethyl-9-(4-(1-methyl-4-(trifluoromethyl)-1H-imidazol-2-yl)benzyl)-8,9-dihydro-7H-purin-6-amine (A-17b)

To a stirred solution of compound A-17a (260 mg, 0.6 mmol) and sodium carbonate (297 mg, 2.8 mmol) in 1,4-dioxane (10 mL) and water (2.5 mL) in a microwave vial, was added (2-isopropylphenyl)boronic acid (138 mg, 0.84 mmol). The reaction mixture was degassed by bubbling with argon for 5 minutes, then Pd(dppf)Cl₂·DCM (46 mg, 0.06 mmol) was added, the mixture was degassed for 2 minutes and stirred at 100° C. for 2 h in a microwave. The reaction mixture was diluted with water (50 mL), extracted with EtOAc (3×50 mL). The combined organic layers were washed with water (50 mL), brine (20 mL), dried (Na₂SO₄), filtered and concentrated under reduced pressure. The afforded crude compound was purified by column chromatography on silica gel, eluted with 2% MeOH/DCM. The residue was purified by prep HPLC on Kromosil C18 (25×150) mm 10μ column using a gradient of 10 mM NH₄HCO₃ in H₂O: MeCN as mobile phase. The residue was further purified by SFC, which gave the title compound (100 mg, 31%) as a solid. LCMS (ES+) m/z 549.64 [M+H]⁺.

¹H NMR (500 MHz, DMSO): δ 7.91 (d, J=1.1 Hz, 1H), 7.67 (d, J=8.2 Hz, 2H), 7.56 (q, J=3.0 Hz, 1H), 7.48 (d, J=7.7 Hz, 2H), 7.40 (q, J=3.0 Hz, 1H), 7.35 (m, J=3.3 Hz, 1H), 7.22 (m, J=3.3 Hz, 1H), 6.11 (d, J=40.0 Hz, 1H), 5.12 (s, 2H), 3.75 (s, 3H), 3.58 (t, J=6.9 Hz, 1H), 3.44 (s, 3H), 2.91 (s, 6H), 1.14 (d, J=6.9 Hz, 6H).

Conditions for Preparative SFC
Column/dimensions: Chiralcel OD-H (250×30 mm), 5p
CO₂: 85.0% Co solvent: 15.0% (30 mM methanolic ammonia in methanol)
Total flow: 70.0 g/min
Back pressure: 90.0 bar
UV: 214 nm
Stack time: 6.6 min
Load/Inj.: 10 mg

### Example A-18

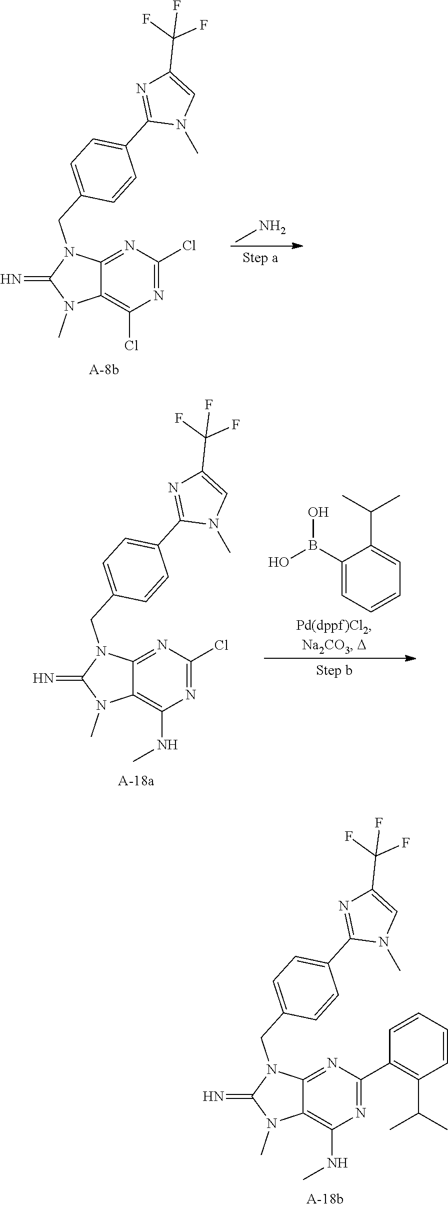

### Step a) 2-chloro-8-imino-N,7-dimethyl-9-(4-(1-methyl-4-(trifluoromethyl)-1H-imidazol-2-yl)benzyl)-8,9-dihydro-7H-purin-6-amine (A-18a)

A solution of compound A-8b (300 mg, 0.7 mmol) in methylamine (2M in MeOH) (3.3 mL, 6.6 mmol) in a sealed tube was stirred at 80° C. for 1 h, then concentrated under reduced pressure, which gave the title compound (270 mg, 89%) as a solid. MS (ES+) m/z 451.32 [M+H]⁺.

### Step b) 8-imino-2-(2-isopropylphenyl)-N, 7-dimethyl-9-(4-(1-methyl-4-(trifluoromethyl)-1H-imidazol-2-yl)benzyl)-8,9-dihydro-7H-purin-6-amine (A-18b)

To a stirred solution of compound A-18a (270 mg, 0.6 mmol) and sodium carbonate (318 mg, 3.0 mmol) in 1,4-dioxane (8.0 mL) and water (2.0 mL) in a microwave vial, was added (2-isopropylphenyl)boronic acid (148 mg, 0.9 mmol). The reaction mixture was degassed by bubbling with argon for 5 minutes, then Pd(dppf)Cl₂·DCM (49 mg, 0.06 mmol) was added, the mixture was degassed for 2 minutes and stirred at 100° C. for 1 h in a microwave. The reaction mixture was diluted with water (50 mL), extracted with EtOAc (3×50 mL). The combined organic layers were washed with water (50 mL), brine (20 mL), dried (Na₂SO₄), filtered and concentrated under reduced pressure. The afforded crude compound was purified by column chromatography on neutral alumina, eluted with 3% MeOH/DCM. The residue was further purified by prep HPLC on Kromosil C18 (25×150) mm 10μ column using a gradient of 10 mM NH₄HCO₃ in H₂O: MeCN as mobile phase. The residue was further purified by SFC, which gave the title compound (60 mg, 18%) as a solid. LCMS (ES+) m/z 535.59 [M+H]⁺.

¹H NMR (500 MHz, DMSO): δ 7.91 (d, J=1.1 Hz, 1H), 7.66 (d, J=8.3 Hz, 2H), 7.53 (q, J=3.0 Hz, 1H), 7.45 (d, J=8.3 Hz, 2H), 7.38 (q, J=3.0 Hz, 1H), 7.33 (m, J=3.3 Hz, 1H), 7.20 (m, J=3.3 Hz, 1H), 6.50 (q, J=4.4 Hz, 1H), 5.78 (s, 1H), 5.10 (s, 2H), 3.75 (s, 3H), 3.60 (m, J=6.4 Hz, 1H), 3.51 (s, 3H), 2.91 (d, J=4.5 Hz, 3H), 1.13 (d, J=6.9 Hz, 6H).

Conditions for Preparative SFC
Column/dimensions: Chiralpak-IG (250×30) mm, 5p
CO₂: 70.0%
Co solvent: 30.0% (30 mM methanolic ammonia in methanol)
Total flow: 100.0 g/min
Back pressure: 100.0 bar
UV: 214 nm
Stack time: 5.0 min
Load/Inj.: 6.2 mg

### Example A-19

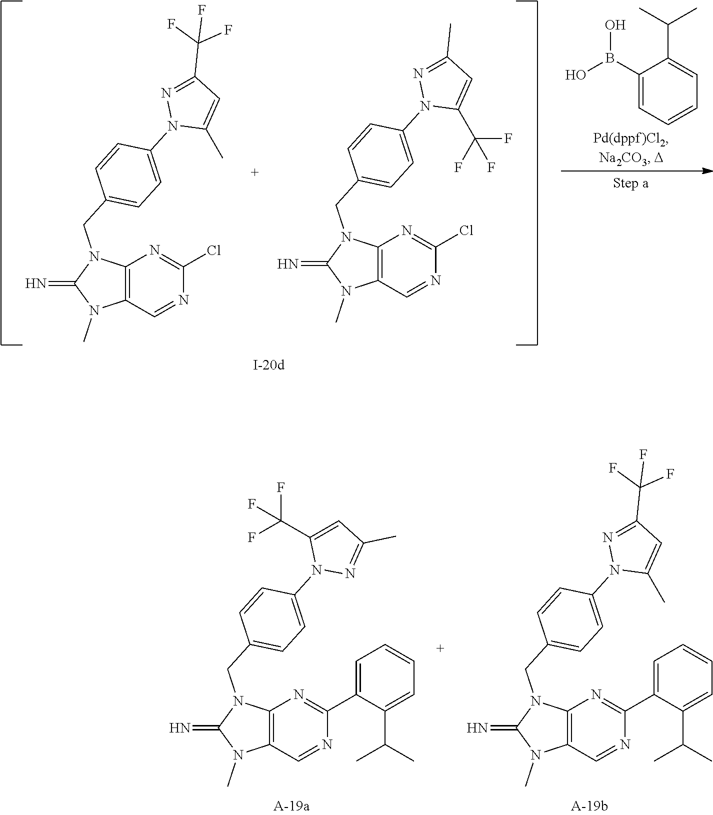

### 2-(2-isopropylphenyl)-7-methyl-9-(4-(3-methyl-5-(trifluoromethyl)-1H-pyrazol-1-yl)benzyl)-7,9-dihydro-8H-purin-8-imine & 2-(2-isopropylphenyl)-7-methyl-9-(4-(5-methyl-3-(trifluoromethyl)-1H-pyrazol-1-yl)benzyl)-7,9-dihydro-8H-purin-8-imine (A-19a & A-19b)

To a stirred solution of compound I-20d (450 mg, 0.53 mmol) and sodium carbonate (226 mg, 2.1 mmol) in 1,4-dioxane (10.0 mL) and water (2.0 mL) in a microwave vial, was added (2-isopropylphenyl)boronic acid (105 mg, 0.64 mmol). The reaction mixture was degassed by bubbling with argon for 5 minutes, then Pd(dppf)Cl₂·DCM (44 mg, 0.05 mmol) was added, the mixture was degassed for 2 minutes and stirred at 120° C. for 2 h in a microwave. The reaction mixture was filtered through the celite bed. The filtrate was concentrated under reduced pressure. The afforded crude compound was purified by column chromatography on silica gel, eluted with 3-4% MeOH/DCM. The impure compound was further purified by column chromatography on silica gel, eluted with 1-3% MeOH/DCM. The residue was purified by prep HPLC on Kromosil C₁₈ (25×150) mm 10μ column using a gradient of 10 mM NH₄HCO₃ in H₂O: MeCN as mobile phase. The residue was further purified by preparative SFC.

### A-19a

Peak-1 was concentrated, which gave the title compound (6 mg, 2%) as a solid. MS (ES+) m/z 506.56 [M+H]⁺.

¹H NMR (500 MHz, DMSO): δ 8.23 (d, J=16.2 Hz, 1H), 7.45 (m, J=5.4 Hz, 5H), 7.37 (m, J=4.0 Hz, 2H), 7.21 (m, J=3.2 Hz, 1H), 6.92 (s, 1H), 6.54 (d, J=57.4 Hz, 1H), 5.19 (d, J=51.6 Hz, 1H), 3.40 (s, 4H), 2.27 (s, 3H), 1.06 (d, J=6.6 Hz, 6H).

### A-19b

Peak-2 was concentrated, which gave the title compound (25 mg, 9%) as a solid. MS (ES+) m/z 506.56 [M+H]⁺.

¹H NMR (500 MHz, DMSO): δ 8.24 (d, J=24.5 Hz, 1H), 7.50 (m, J=7.3 Hz, 5H), 7.37 (m, J=4.3 Hz, 2H), 7.22 (m, J=3.2 Hz, 1H), 6.74 (s, 1H), 6.54 (d, J=48.4 Hz, 1H), 5.19 (d, J=50.9 Hz, 1H), 3.39 (d, J=15.6 Hz, 4H), 2.31 (d, J=0.5 Hz, 3H), 1.09 (d, J=6.5 Hz, 6H).

Conditions for Preparative SFC
Column/dimensions: Chiralpak IG (30×250) mm, 5p
% CO₂: 75.0%
% Co solvent: ²5.0% (methanol)
Total Flow: 70.0 g/min
Back Pressure: 100.0 bar
UV: 214 nm
Stack time: 6.0 min
Load/Inj: 3.05 mg

### Example A-20

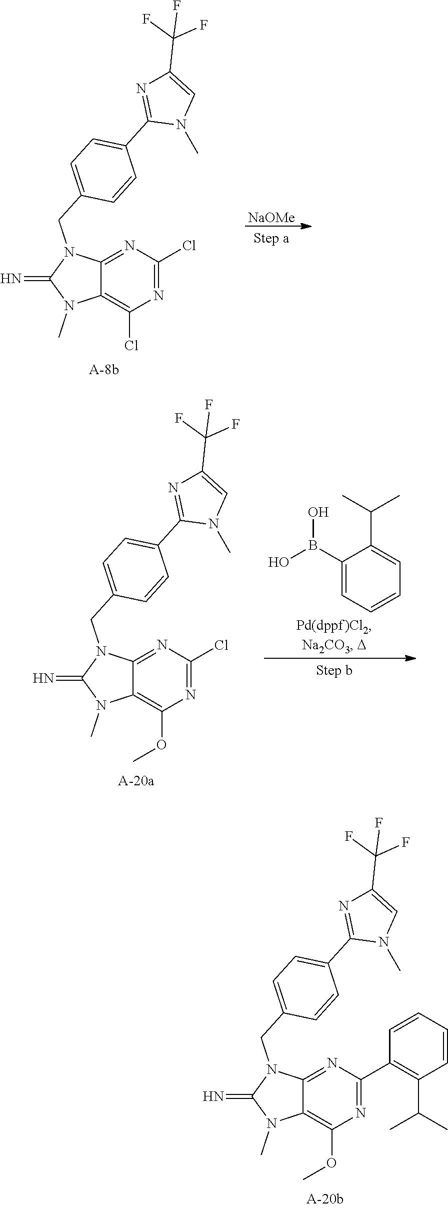 [SMILES: Cn1cc(C(F)(F)F)nc1-c1ccc(Cn2c(=N)n(C)c3c(Cl)nc(Cl)nc32)cc1 ; COc1nc(Cl)nc2c1n(C)c(=N)n2Cc1ccc(-c2nc(C(F)(F)F)cn2C)cc1 ; CC(C)c1ccccc1B(O)O ; "NaOMe" ; COc1nc(-c2ccccc2C(C)C)nc2c1n(C)c(=N)n2Cc1ccc(-c2nc(C(F)(F)F)cn2C)cc1]

### Step a) 2-chloro-6-methoxy-7-methyl-9-(4-(1-methyl-4-(trifluoromethyl)-1H-imidazol-2-yl)benzyl)-7,9-dihydro-8H-purin-8-imine (A-20a)

NaOMe (2.5M in MeOH) (0.25 mL, 1.1 mmol) was added at 0° C. to a stirred solution of compound A-8b (300 mg, 0.52 mmol) in MeOH (10 mL). The mixture was stirred at 0° C. for 10 min and at rt for 1 h. Ice water was added to the residue and extracted with EtOAc. The combined organic layer was washed with brine, dried (Na₂SO₄), filtered and concentrated under reduced pressure, which gave the title compound (300 mg, 97%) as a solid. MS (ES+) 452.30 [M+H]⁺.

### Step b) 2-(2-isopropylphenyl)-6-methoxy-7-methyl-9-(4-(1-methyl-4-(trifluoromethyl)-1H-imidazol-2-yl)benzyl)-7,9-dihydro-8H-purin-8-imine (A-20b)

(2-isopropylphenyl) boronic acid (166 mg, 1.0 mmol) and sodium carbonate (161 mg, 1.5 mmol) were added to a stirred solution of compound A-20a (300 mg, 0.51 mmol) in 1,4-dioxane (13 mL) and water (3 mL) in a microwave vial. The reaction mixture was degassed by bubbling with argon for 15 min, then Pd(dppf)Cl₂·DCM (37 mg, 0.05 mmol) was added and the reaction mixture was degassed with argon for 2 minutes and stirred at 120° C. for 2 h in a microwave. The reaction mixture was filtered through the celite bed. The filtrate was concentrated under reduced pressure. The afforded crude compound was combined with another batch and purified by column chromatography on silica gel, eluted with 7% MeOH/DCM. The residue was purified by prep IPLC on Kromosil C18 (25×150) mm 10μ column using a gradient of 10 mM NH₄HCO₃ in H₂O: MeCN as mobile phase, which gave the title compound (80 mg) as a solid. MS (ES+) m/z 536.60 [M+H]⁺.

¹H NMR (500 MHz, DMSO): δ 7.91 (d, J=1.1 Hz, 1H), 7.67 (d, J=8.3 Hz, 2H), 7.59 (q, J=3.0 Hz, 1H), 7.47 (d, J=8.1 Hz, 2H), 7.42 (q, J=3.0 Hz, 1H), 7.38 (m, J=3.3 Hz, 1H), 7.24 (m, J=3.2 Hz, 1H), 6.12 (s, 1H), 5.16 (s, 2H), 4.02 (s, 3H), 3.75 (s, 3H), 3.57 (m, J=6.9 Hz, 1H), 3.48 (s, 3H), 1.14 (d, J=6.9 Hz, 6H).

### Example A-21

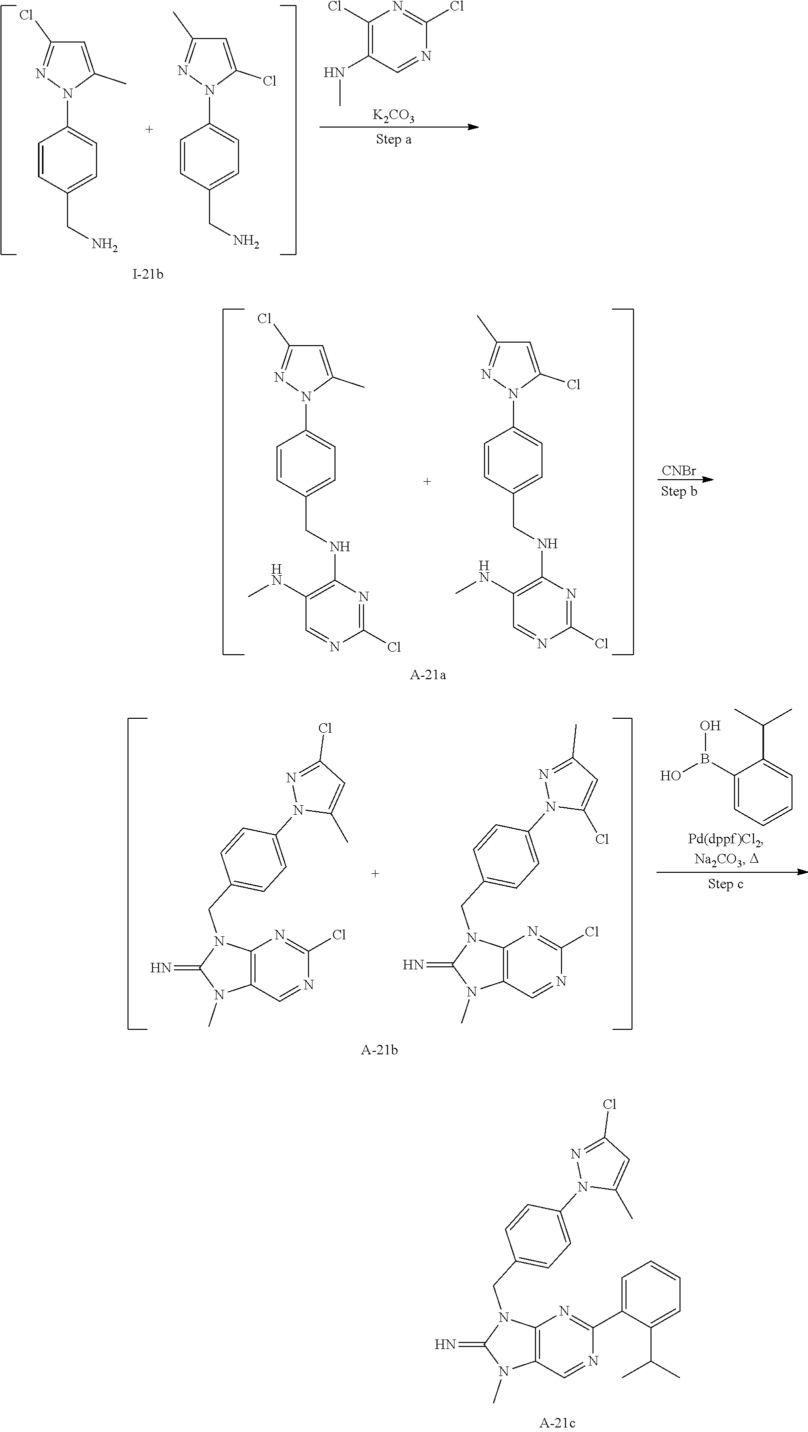

### Step a) 2-chloro-N4-(4-(3-chloro-5-methyl-1H-pyrazol-1-yl)benzyl)-N5-methylpyrimidine-4,5-diamine & 2-chloro-N4-(4-(5-chloro-3-methyl-1H-pyrazol-1-yl)benzyl)-N5-methylpyrimidine-4,5-diamine (A-21a)

K₂CO₃ (875 mg, 6.8 mmol) was added at 0° C. to a stirred solution compound I-21b (1 g, 2.3 mmol) and of 2,4-dichloro-N-methylpyrimidin-5-amine (600 mg, 3.4 mmol) in DMF (10 mL). The resulting reaction mixture was stirred at 100° C. for 16 h, then quenched with ice water and extracted with EtOAc (2×100 mL). The combined organic layer was washed with brine (100 mL), dried (Na₂SO₄), filtered and concentrated under reduced pressure. The crude product was purified by column chromatography on silica-gel eluted with a gradient of 20% EtOAc in pet ether, which gave the inseparable mixture of title compounds (350 mg, 18%) as a semi-solid.

LCMS (ES+) m/z 363.25 [M+H]⁺.

### Step b) 2-chloro-9-(4-(3-chloro-5-methyl-1H-pyrazol-1-yl)benzyl)-7-methyl-7,9-dihydro-8H-purin-8-imine & 2-chloro-9-(4-(5-chloro-3-methyl-1H-pyrazol-1-yl)benzyl)-7-methyl-7,9-dihydro-8H-purin-8-imine (A-21b)

Cyanogen bromide (255 mg, 2.4 mmol) was added at 0° C. to a stirred solution of compound A-21a (350 mg, 0.5 mmol) in EtOH (10 mL). The resulting mixture was stirred at 90° C. for 16 h, then concentrated under reduced pressure. The crude compound was purified by column chromatography on silica gel and eluted with 10% MeOH/DCM, which gave the inseparable mixture of title compounds (150 mg, 44%) as a solid. LCMS (ES+) m/z 388.25 [M+H]⁺.

### Step c) 9-(4-(3-chloro-5-methyl-1H-pyrazol-1-yl)benzyl)-2-(2-isopropylphenyl)-7-methyl-7,9-dihydro-8H-purin-8-imine (A-21c)

Sodium carbonate (51.2 mg, 0.5 mmol) was added to a stirred solution of compound A-21b (150 mg, 0.2 mmol) and (2-isopropylphenyl) boronic acid (39 mg, 0.23 mmol) in 1,4-dioxane (5 mL) and water (1 mL) in a microwave vial. The reaction mixture was degassed by bubbling with argon for 10 min, then Pd(dppf)Cl₂·DCM (8 mg, 0.01 mmol) was added and the mixture was degassed for 10 minutes and stirred at 120° C. for 2 h in a microwave. The reaction mixture was filtered through the celite bed, filtrate was concentrated under reduced pressure. The afforded crude compound was combined with another batch and purified by column chromatography on silica gel, eluted with 10% MeOH/DCM. The residue was purified by prep HPLC on Kromosil C18 (25×150) mm 10μ column using a gradient of 10 mM NH₄HCO₃ in H₂O: MeCN as mobile phase. The impure compound was further purified by preparative SFC, which gave the title compound (8 mg) as a solid. MS (ES+) m/z 472.51 [M+H]⁺.

¹H NMR (500 MHz, DMSO): δ 8.24 (s, 1H), 7.48 (q, J=2.9 Hz, 5H), 7.38 (m, J=3.4 Hz, 2H), 7.22 (m, J=3.2 Hz, 1H), 6.52 (s, 1H), 6.36 (d, J=0.6 Hz, 1H), 5.16 (s, 2H), 3.43 (d, J=6.9 Hz, 1H), 3.39 (s, 3H), 2.27 (d, J=0.4 Hz, 3H), 1.08 (d, J=6.9 Hz, 6H).

Conditions for Preparative SFC
Column/dimensions: Chiralpak IG (30×250) mm, 5p
% CO₂: 65.0%
% Co solvent: ³5.0% (0.5% diethylamine in methanol)
Total Flow: 70.0 g/min
Back Pressure: 90.0 bar
UV: 214 nm
Stack time: 17 min
Load/Inj: 2.9 mg

### Example A-22

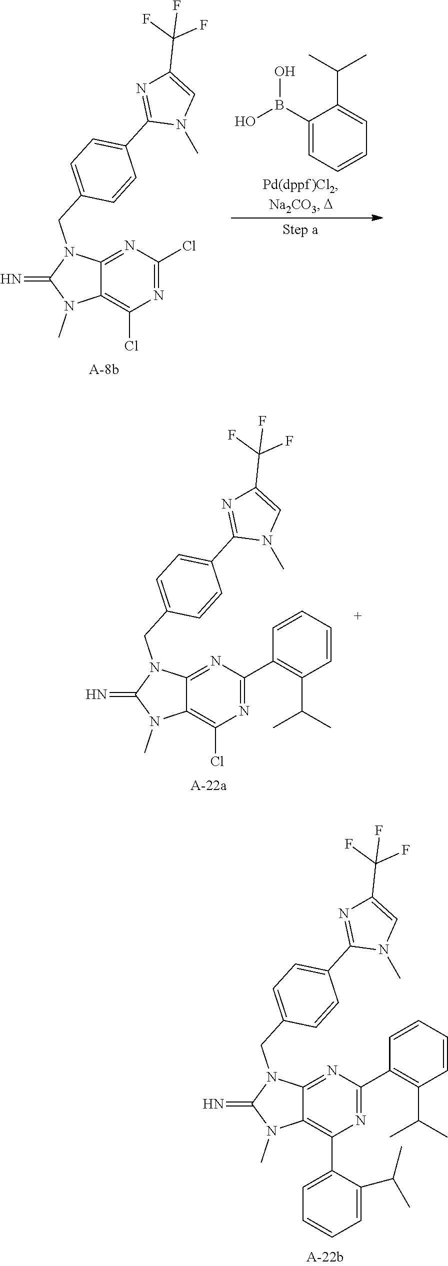 [SMILES: Cn1cc(C(F)(F)F)nc1-c1ccc(Cn2c(=N)n(C)c3c(Cl)nc(Cl)nc32)cc1 ; CC(C)c1ccccc1B(O)O ; CC(C)c1ccccc1-c1nc(Cl)c2c(n1)n(Cc1ccc(-c3nc(C(F)(F)F)cn3C)cc1)c(=N)n2C ; CC(C)c1ccccc1-c1nc(-c2ccccc2C(C)C)c2c(n1)n(Cc1ccc(-c3nc(C(F)(F)F)cn3C)cc1)c(=N)n2C]

### 6-chloro-2-(2-isopropylphenyl)-7-methyl-9-(4-(1-methyl-4-(trifluoromethyl)-1H-imidazol-2-yl)benzyl)-7,9-dihydro-8H-purin-8-imine & 2,6-bis(2-isopropylphenyl)-7-methyl-9-(4-(1-methyl-4-(trifluoromethyl)-1H-imidazol-2-yl)benzyl)-7,9-dihydro-8H-purin-8-imine (A-22a & A-22b)

(2-isopropylphenyl) boronic acid (67 mg, 0.41 mmol) and sodium carbonate (216 mg, 2.04 mmol) were added to a stirred solution of compound A-8b (200 mg, 0.41 mmol) in 1,4-dioxane (16 mL) and water (4 mL) in a microwave vial. The reaction mixture was degassed by bubbling with argon for 15 min then Pd(dppf)Cl₂·DCM (30 mg, 0.04 mmol) was added and the mixture was degassed for 5 minutes and stirred at 100° C. for 90 minutes in a microwave. The reaction mixture was filtered through the celite bed. The filtrate was concentrated under reduced pressure. The afforded crude compound was combined with another batch and purified by column chromatography on silica gel, eluted with 7% MeOH/DCM. The residue was further purified by prep HPLC on Kromosil C18 (25×150) mm 10μ column using a gradient of 10 mM NH₄HCO₃ in H₂O: MeCN as mobile phase.

A-22a: Peak-2 was concentrated and lyophilised, which gave the title compound (30 mg) as a solid. MS (ES+) m/z 540.56 [M+H]⁺.

¹H NMR (500 MHz, DMSO): δ 7.92 (s, 1H), 7.68 (t, J=7.4 Hz, 2H), 7.48 (m, J=5.9 Hz, 3H), 7.41 (t, J=8.0 Hz, 2H), 7.25 (m, J=4.1 Hz, 1H), 6.78 (d, J=23.5 Hz, 1H), 5.20 (d, J=54.2 Hz, 2H), 3.75 (s, 3H), 3.59 (d, J=6.1 Hz, 3H), 3.43 (t, J=5.8 Hz, 1H), 1.10 (t, J=6.3 Hz, 6H).

A-22b: Peak-3 was concentrated and lyophilised, which gave the title compound (30 mg) as a solid. MS (ES+) m/z 624.71 [M+H]⁺.

¹H NMR (500 MHz, DMSO): δ 7.93 (s, 1H), 7.71 (s, 2H), 7.50 (m, J=3.1 Hz, 5H), 7.35 (m, J=5.2 Hz, 3H), 7.23 (m, J=4.0 Hz, 1H), 6.50 (d, J=87.0 Hz, 1H), 5.23 (d, J=48.1 Hz, 2H), 3.77 (s, 3H), 3.57 (d, J=6.8 Hz, 1H), 2.94 (s, 1H), 2.82 (s, 3H), 1.15 (t, J=7.6 Hz, 9H), 1.05 (d, J=6.7 Hz, 3H).

### Example A-23

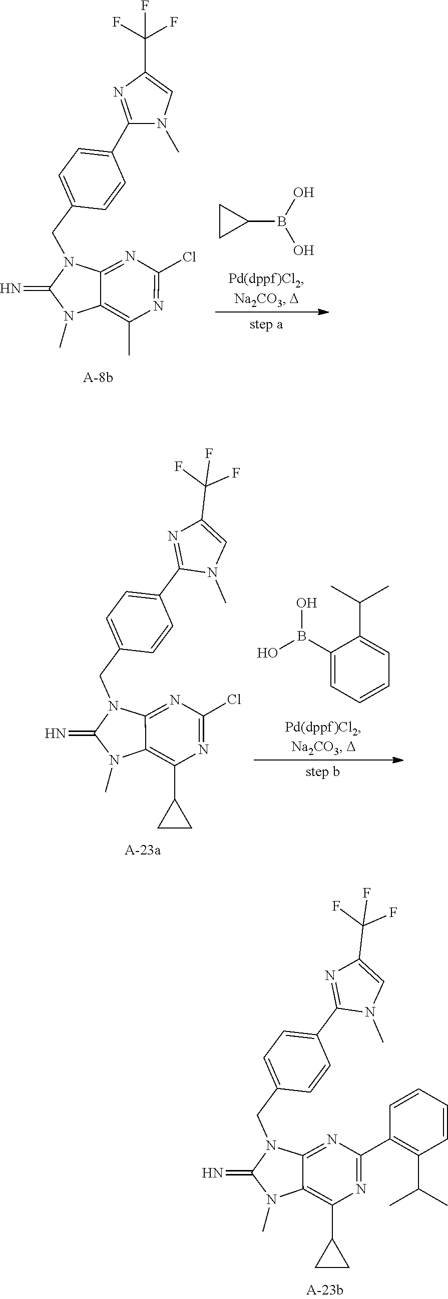

### Step a) 2-chloro-6-cyclopropyl-7-methyl-9-(4-(1-methyl-4-(trifluoromethyl)-1H-imidazol-2-yl)benzyl)-7,9-dihydro-8H-purin-8-imine (A-23a)

Cyclopropylboronic acid (70 mg, 0.82 mmol) and sodium carbonate (130 mg, 1.2 mmol) were added to a stirred solution of compound A-8b (200 mg, 0.41 mmol) in 1,4-dioxane (8 mL) and water (2 mL) in a microwave vial. The reaction mixture was degassed by bubbling with argon for 15 min then Pd(dppf)Cl₂·DCM (30 mg, 0.04 mmol) was added and the mixture was degassed for 5 minutes and stirred at 100° C. for 3 h in a microwave. The reaction mixture was filtered through the celite bed and the filtrate was concentrated under reduced pressure. The afforded crude compound was purified by column chromatography on silica gel, eluted with 8% MeOH/DCM, which gave the title compound (150 mg, 63%) as a solid. MS (ES+) m/z 462.46 [M+H]⁺.

### Step b) 6-cyclopropyl-2-(2-isopropylphenyl)-7-methyl-9-(4-(1-methyl-4-(trifluoromethyl)-1H-imidazol-2-yl)benzyl)-7,9-dihydro-8H-purin-8-imine (A-23b)

(2-isopropylphenyl)boronic acid (85 mg, 0.52 mmol) and sodium carbonate (83 mg, 0.8 mmol) were added to a stirred solution of compound A-23a (150 mg, 0.3 mmol) in 1,4-dioxane (8 mL) and water (2 mL) in a sealed tube. The reaction mixture was degassed by bubbling with argon for 15 min then Pd(dppf)Cl₂·DCM (19 mg, 0.03 mmol) was added and the mixture was degassed for 5 minutes, then stirred at 100° C. for 16 h in a sealed tube. The reaction mixture was filtered through the celite bed and the filtrate was concentrated under reduced pressure. The afforded crude compound was purified by column chromatography on silica gel, eluted with 7% MeOH/DCM. The residue was purified by prep HPLC on Kromosil C18 (25×150) mm 10p column using a gradient of 10 mM NH₄HCO₃ in H₂O: MeCN as mobile phase (Peak-2), which gave the title compound (40 mg, 27%) as a solid. MS (ES+) m/z 546.65 [M+H]⁺.

¹H NMR (500 MHz, DMSO): δ 7.91 (d, J=0.8 Hz, 1H), 7.67 (s, 2H), 7.48 (q, J=3.0 Hz, 3H), 7.36 (m, J=3.8 Hz, 2H), 7.21 (m, J=3.3 Hz, 1H), 6.35 (s, 1H), 5.17 (d, J=46.7 Hz, 2H), 3.75 (s, 3H), 3.64 (s, 3H), 3.47 (t, J=6.4 Hz, 1H), 1.11 (d, J=6.9 Hz, 10H).

### Example A-24

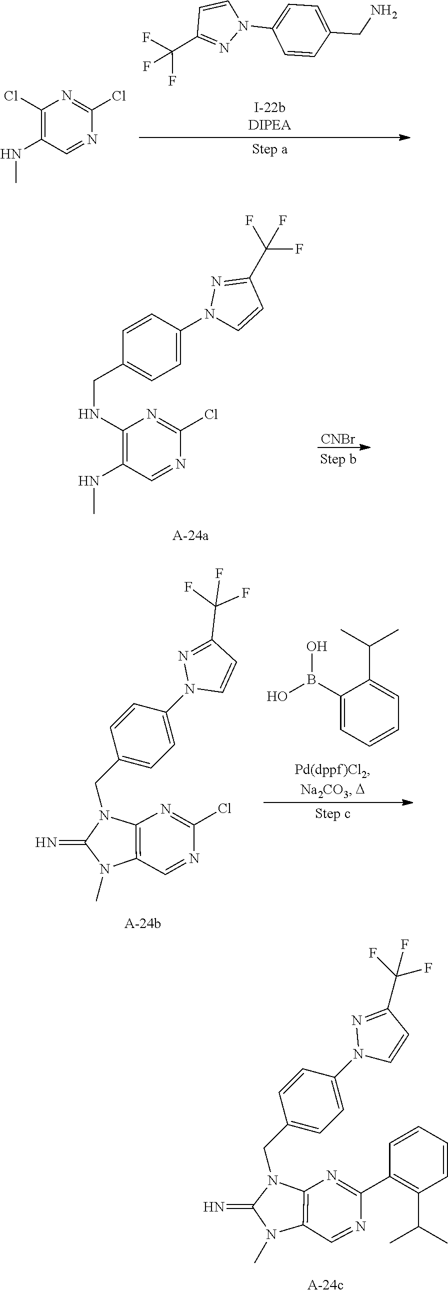

### Step a) 2-chloro-N5-methyl-N4-(4-(3-(trifluoromethyl)-1H-pyrazol-1-yl)benzyl)pyrimidine-4,5-diamine (A-24a)

DIPEA (2.9 mL, 16.6 mmol) was added to a stirred solution of compound I-22b (800 mg, 3.3 mmol) in DMF (20 mL) at rt and stirred at rt for 5 min, then 2,4-dichloro-N-methylpyrimidin-5-amine (590 mg, 3.3 mmol) was added at 0° C. and the resulting mixture was stirred for 16 h at 80° C. Water (80 mL) was added and the mixture was extracted with EtOAc (3×50 mL). The organic layer was washed with brine (100 mL), dried (Na₂SO₄), filtered and concentrated under reduced pressure. The afforded residue was purified by column chromatography on neutral alumina, eluted with 70% EtOAc in pet ether, which gave the title compound (700 mg, 52%) as a solid. LCMS (ES+) 383.21 [M+H]⁺.

### Step b) 2-chloro-7-methyl-9-(4-(3-(trifluoromethyl)-1H-pyrazol-1-yl)benzyl)-7,9-dihydro-8H-purin-8-imine (A-24b)

Cyanogen bromide (1.8 g, 17.4 mmol) was added at 0° C. to a stirred solution of compound A-24a (700 mg, 1.7 mmol) in EtOH (20 mL). The resulting mixture was stirred at 80° C. for 16 h, then cooled to rt. The precipitated solid was filtered, washed with pet ether and dried, which gave the title compound (400 mg, 53%) as a solid. LCMS (ES+) 408.22 [M+H]⁺.

### Step c) 2-(2-isopropylphenyl)-7-methyl-9-(4-(3-(trifluoromethyl)-1H-pyrazol-1-yl)benzyl)-7,9-dihydro-8H-purin-8-imine (A-24c)

(2-isopropylphenyl) boronic acid (161 mg, 1.0 mmol)) was added to a stirred solution of compound A-24b (200 mg, 0.5 mmol) and sodium carbonate (260 mg, 2.5 mmol) in 1,4-dioxane (16 mL) and water (4 mL) in a microwave vial. The reaction mixture was degassed by bubbling with argon for 10 minutes then Pd(dppf)Cl₂·DCM (40 mg, 0.05 mmol) was added and the mixture was degassed for 2 minutes and stirred at 100° C. for 1 h in a microwave. The reaction mixture was diluted with water (50 mL) and extracted with EtOAc (3×50 mL). The combined organic layers were washed with water (80 mL), brine (80 mL), dried (Na₂SO₄), filtered and concentrated under reduced pressure. The afforded crude compound was purified by column chromatography on neutral alumina, eluted with 2% MeOH/DCM. The residue was further purified by prep HPLC on Kromosil C18 (25×150) mm 10μ column using a gradient of 10 mM NH₄HCO₃ in H₂O: MeCN as mobile phase, which gave the title compound (115 mg, 47%) as a solid. LCMS (ES+) m/z 492.54 [M+H]⁺.

¹H NMR (500 MHz, DMSO): δ 8.68 (d, J=1.6 Hz, 1H), 8.23 (s, 1H), 7.83 (d, J=8.6 Hz, 2H), 7.50 (m, J=6.0 Hz, 3H), 7.37 (m, J=3.6 Hz, 2H), 7.23 (m, J=3.2 Hz, 1H), 7.03 (d, J=2.5 Hz, 1H), 6.54 (d, J=14.0 Hz, 1H), 5.15 (s, 2H), 3.44 (t, J=6.9 Hz, 1H), 3.38 (s, 3H), 1.09 (d, J=6.9 Hz, 6H).

### Example A-25

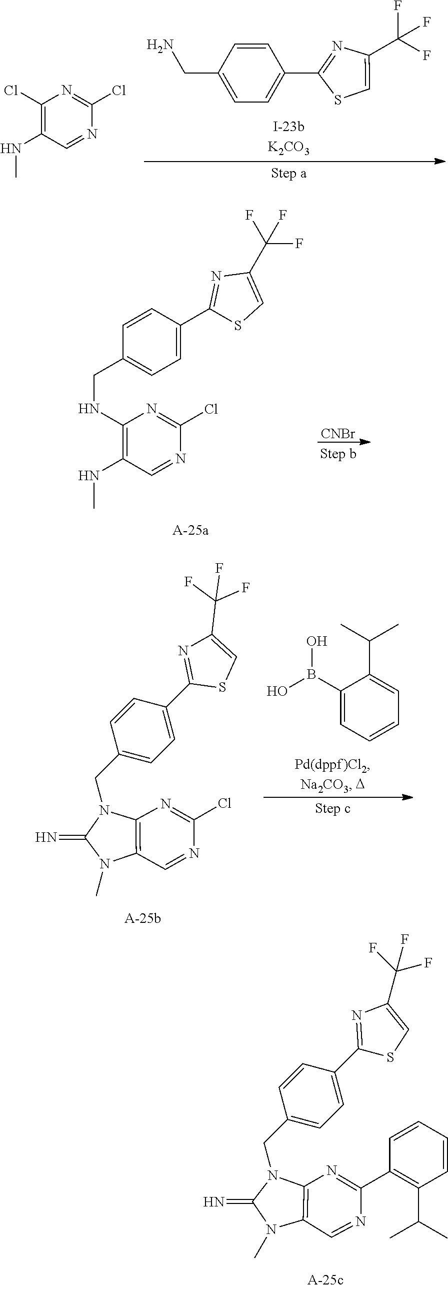

### Step a) 2-chloro-N5-methyl-N4-(4-(4-(trifluoromethyl)thiazol-2-yl)benzyl)pyrimidine-4,5-diamine (A-25a)

K₂CO₃ (1.4 g, 10 mmol) and compound I-23b (718 mg, 2.8 mmol) were added at rt to a stirred solution of 2,4-dichloro-N-methylpyrimidin-5-amine (450 mg, 2.5 mmol) in DMF (15 mL). The resulting reaction mixture was stirred at 90° C. for 12 h, then dissolved in water and extracted with EtOAc. The combined organic layers were washed with brine, dried (Na₂SO₄), filtered and concentrated under reduced pressure. The crude product was purified by column chromatography on silica-gel eluted with a gradient of 65-70% EtOAc in pet ether, which gave the mixture of title compounds (750 mg, 52%) as a semi-solid. LCMS (ES+) m/z 400.22 [M+H]⁺.

### Step b) 2-chloro-7-methyl-9-(4-(4-(trifluoromethyl)thiazol-2-yl)benzyl)-7,9-dihydro-8H-purin-8-imine (A-25b)

Cyanogen bromide (716 mg, 6.8 mmol) was added at rt to a stirred solution of compound A-25a (750 mg, 1.7 mmol) in EtOH (10 mL). The resulting mixture was stirred at 80° C. for 8 h, then concentrated under reduced pressure. The afforded residue was purified by column chromatography on silica gel, eluted with 3-6% MeOH in DCM, which gave the title compound (500 mg, 29%) as a solid. LCMS (ES+) 425.35 [M+H]⁺.

### Step c) 2-(2-isopropylphenyl)-7-methyl-9-(4-(4-(trifluoromethyl)thiazol-2-yl)benzyl)-7,9-dihydro-8H-purin-8-imine (A-25c)

(2-isopropylphenyl) boronic acid (139 mg, 0.85 mmol)) was added to a stirred solution of compound A-25b (300 mg, 0.71 mmol) and sodium carbonate (300 mg, 2.8 mmol) in 1,4-dioxane (10 mL) and water (2 mL) in a microwave vial. The reaction mixture was degassed by bubbling with argon for 5 minutes then Pd(dppf)Cl₂·DCM (58 mg, 0.07 mmol) was added and the mixture was degassed for 2 minutes and stirred at 120° C. for 2 h in a microwave. The reaction mixture was filtered through the celite bed and the filtrate was concentrated under reduced pressure. The afforded crude compound was purified by column chromatography on neutral alumina, eluted with 4-6% MeOH/DCM. The residue was further purified by prep HPLC on Kromosil C18 (25×150) mm 10μ column using a gradient of 10 mM NH₄HCO₃ in H₂O: MeCN as mobile phase, which gave the title compound (45 mg, 12%) as a solid. LCMS (ES+) m/z 509.55 [M+H]⁺.

¹H NMR (500 MHz, DMSO): δ 8.54 (s, 1H), 8.24 (t, J=15.0 Hz, 1H), 7.95 (d, J=7.5 Hz, 2H), 7.49 (m, J=5.1 Hz, 3H), 7.36 (m, J=3.3 Hz, 2H), 7.22 (m, J=2.3 Hz, 1H), 6.53 (d, J=37.3 Hz, 1H), 5.18 (d, J=51.1 Hz, 2H), 3.39 (d, J=12.1 Hz, 3H), 1.06 (d, J=6.7 Hz, 6H).

### Example A-26

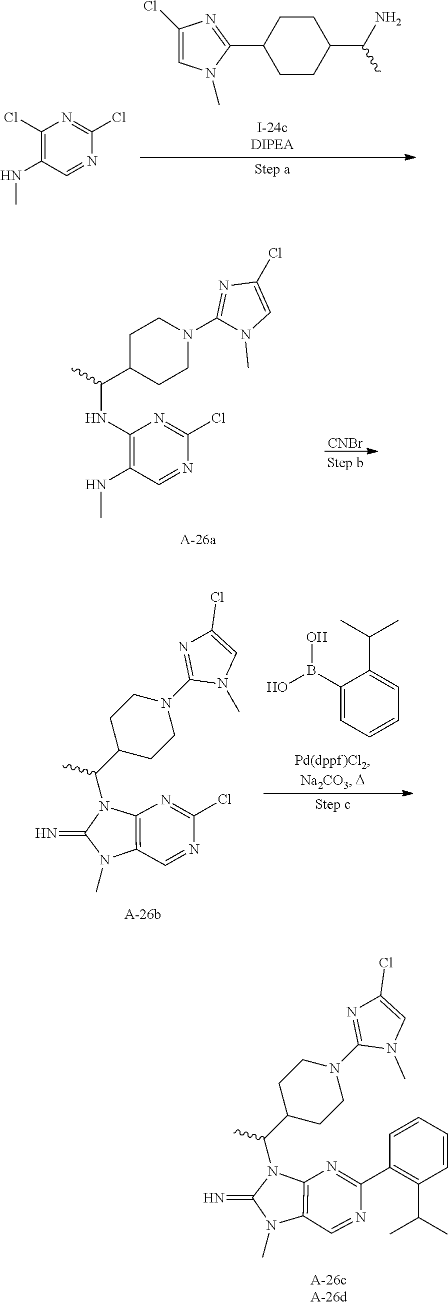

### Step a) 2-chloro-N4-(1-(1-(4-chloro-1-methyl-1H-imidazol-2-yl)piperidin-4-yl)ethyl)-N₅-methylpyrimidine-4,5-diamine (A-26a)

DIPEA (1.7 mL, 9.5 mmol) was added to a stirred solution of compound I-24c (550 mg, 1.9 mmol) in DMF (10 mL) at rt and stirred at rt for 5 min, then 2,4-dichloro-N-methylpyrimidin-5-amine (340 mg, 1.9 mmol) was added at 0° C. and the resulting mixture was stirred for 16 h at 80° C. Water (100 mL) was added and the mixture was extracted with EtOAc (3×80 mL). The organic layer was washed with brine (100 mL), dried (Na₂SO₄), filtered and concentrated under reduced pressure. The afforded residue was purified by column chromatography on neutral alumina, eluted with 50% EtOAc in pet ether, which gave the title compound (500 mg, 65%) as a solid. LCMS (ES+) 384.26 [M+H]⁺.

### Step b) 2-chloro-9-(1-(1-(4-chloro-1-methyl-H-imidazol-2-yl)piperidin-4-yl)ethyl)-7-methyl-7,9-dihydro-8H-purin-8-imine (A-26b)

Cyanogen bromide (276 mg, 2.6 mmol) was added at 0° C. to a stirred solution of compound A-26a (400 mg, 1.0 mmol) in EtOH (20 mL). The resulting mixture was stirred at 80° C. for 4 h, then concentrated under reduced pressure. The residue was dissolved in water (pH was adjusted to 7 by adding saturated NaHCO₃ solution) and extracted with EtOAc (3×100 mL). The combined organic layers were washed with brine, dried (Na₂SO₄), filtered and concentrated under reduced pressure, which gave the title compound (350 mg, 43%) as a semi-solid. LCMS (ES+) m/z 409.44 [M+H]⁺.

### Step c) 9-(1-(1-(4-chloro-1-methyl-1H-imidazol-2-yl)piperidin-4-yl)ethyl)-2-(2-isopropylphenyl)-7-methyl-7,9-dihydro-8H-purin-8-imine (A-26c & A-26d)

(2-isopropylphenyl) boronic acid (320 mg, 2.0 mmol) was added to a stirred solution of compound A-26b (400 mg, 1.0 mmol) and sodium carbonate (518 mg, 4.9 mmol) in 1,4-dioxane (20 mL) and water (5 mL) in a microwave vial. The reaction mixture was degassed by bubbling with argon for 10 minutes then Pd(dppf)Cl₂·DCM (80 mg, 0.1 mmol) was added and the mixture was degassed for 2 minutes, then stirred at 100° C. for 1 h in a microwave. The reaction mixture was diluted with water (50 mL) extracted with EtOAc (3×50 mL). The combined organic layers were washed with water (100 mL), brine (100 mL), dried (Na₂SO₄), filtered and concentrated. The afforded crude compound was combined with another batch and purified by column chromatography on neutral alumina, eluted with 5% MeOH/DCM. The residue was further purified by prep HPLC on Kromosil C18 (25×150) mm 10μ column using a gradient of 10 mM NH₄HCO₃ in H₂O: MeCN as mobile phase. The racemate was separated by Chiral SFC.

A-26c: Peak-1 was concentrated, which gave the title compound (27 mg) as a solid. MS (ES+) m/z 493.59 [M+H]⁺.

¹H NMR (500 MHz, DMSO): δ 8.18 (d, J=19.0 Hz, 1H), 7.51 (q, J=3.0 Hz, 1H), 7.44 (q, J=3.0 Hz, 1H), 7.38 (m, J=3.3 Hz, 1H), 7.24 (m, J=3.2 Hz, 1H), 6.92 (s, 1H), 6.34 (s, 1H), 4.39 (q, J=5.8 Hz, 1H), 3.59 (t, J=6.7 Hz, 1H), 3.37 (s, 6H), 3.23 (d, J=2.7 Hz, 1H), 3.14 (t, J=12.2 Hz, 1H), 2.67 (m, J=5.9 Hz, 1H), 2.46 (s, 2H), 1.91 (d, J=10.7 Hz, 1H), 1.49 (d, J=6.9 Hz, 3H), 1.31 (m, J=11.0 Hz, 3H), 1.22 (d, J=6.7 Hz, 3H), 1.16 (d, J=6.7 Hz, 3H).

A-26d: Peak-2 was concentrated and lyophilised, which gave the title compound (26 mg) as a solid. MS (ES+) m/z 493.59 [M+H]⁺.

¹H NMR (500 MHz, DMSO): δ 8.18 (d, J=18.8 Hz, 1H), 7.51 (q, J=3.0 Hz, 1H), 7.44 (q, J=3.0 Hz, 1H), 7.38 (m, J=3.3 Hz, 1H), 7.24 (m, J=3.2 Hz, 1H), 6.92 (s, 1H), 6.34 (s, 1H), 4.39 (q, J=5.8 Hz, 1H), 3.59 (t, J=6.7 Hz, 1H), 3.37 (s, 6H), 3.25 (m, J=6.2 Hz, 1H), 3.13 (d, J=11.7 Hz, 1H), 2.66 (d, J=20.6 Hz, 1H), 2.45 (s, 2H), 1.90 (s, 1H), 1.49 (d, J=6.9 Hz, 3H), 1.32 (m, J=10.4 Hz, 3H), 1.22 (d, J=6.7 Hz, 3H), 1.16 (d, J=6.8 Hz, 3H).

Preparative Chiral SFC Conditions
Column/dimensions: Chiralpak-IC (250×30) mm, 5p
CO₂: 60.0%
Co solvent: 40.0% (30 mM methanolic ammonia in methanol)
Total flow: 70.0 g/min
Back pressure: 90.0 bar
UV: 214 nm
Stack time: 7.0 min
Load/Inj.: 4.4 mg

### Example A-27

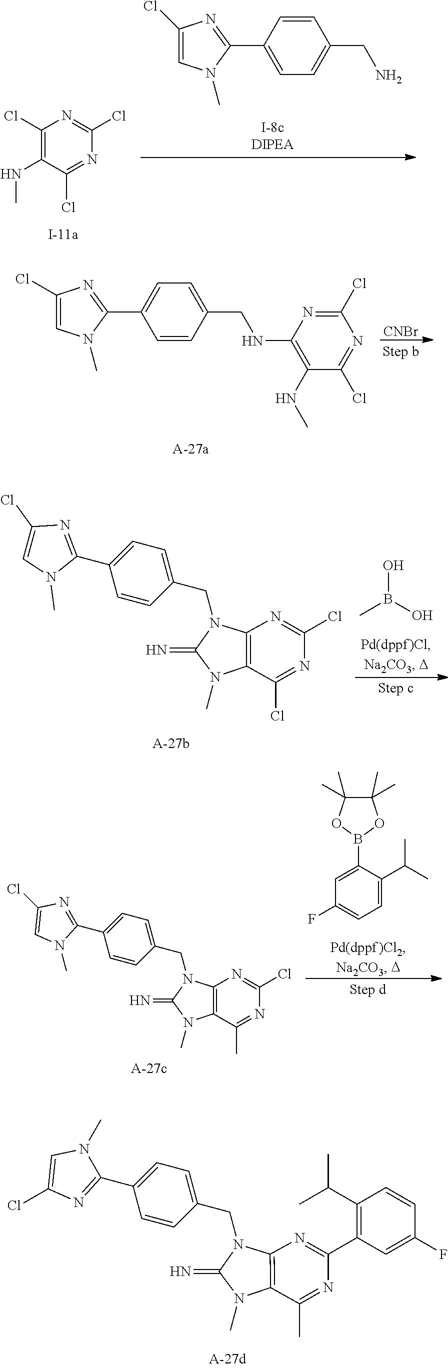

### Step a) 2,6-dichloro-N4-(4-(4-chloro-1-methyl-1H-imidazol-2-yl)benzyl)-N5-methylpyrimidine-4,5-diamine (A-27a)

DIPEA (12.6 mL, 72.1 mmol) was added to a stirred solution of compound I-11a (5.5 g, 24.1 mmol) in THE (200 mL) at rt for 5 min, then compound I-8c (5.8 g, 22.6 mmol) was added at 0° C. and the resulting mixture was stirred for 16 h at 80° C., then concentrated under reduced pressure. Ice cold water was added to the residue and extracted with EtOAc (3×150 mL). The organic layer was washed with brine (100 mL), dried (Na₂SO₄), filtered and concentrated under reduced pressure. The afforded residue was purified by column chromatography on silica gel, eluted with 60% EtOAc in pet ether, which gave the title compound (7.2 g, 73%) as a solid. LCMS (ES+) 397.24 [M+H]⁺.

### Step b) 2,6-dichloro-9-(4-(4-chloro-1-methyl-1H-imidazol-2-yl)benzyl)-7-methyl-7,9-dihydro-8H-purin-8-imine (A-27b)

Cyanogen bromide (2.6 g, 24.6 mmol) was added at 0° C. to a stirred solution of compound A-27a (4 g, 9.9 mmol) in EtOH (80 mL). The resulting mixture was stirred at 80° C. for 16 h, then the precipitated solid was filtered, washed with EtOH (10 mL) and dried, which gave the title compound (2.2 g, 46%) as a solid. LCMS (ES+) 422.36 [M+H]⁺.

### Step c) 2-chloro-9-(4-(4-chloro-1-methyl-1H-imidazol-2-yl)benzyl)-6,7-dimethyl-7,9-dihydro-8H-purin-8-imine (A-27c)

A stirred solution of compound A-27b (1.5 g, 3 mmol) and methyl boronic acid (280 mg, 4.7 mmol) in 1,4-dioxane (15 mL) and water (5 mL) was degassed by bubbling with argon for 10 minutes, then sodium carbonate (1.7 g, 16 mmol) and Pd(dppf)Cl₂·DCM (230 mg, 0.31 mmol) were added and the reaction mixture was stirred at 90° C. for 16 h. The reaction mixture was diluted with water, filtered through the celite bed. The filtrate was extracted with EtOAc. The combined organic layer was washed with brine, dried (Na₂SO₄), filtered and concentrated under reduced pressure. The afforded crude compound was purified by column chromatography on silica gel, eluted with 5% MeOH/DCM, which gave the title compound (600 mg, 41%) as a solid. LCMS (ES+) m/z 402.36 [M+H]⁺.

### Step d) 9-(4-(4-chloro-1-methyl-1H-imidazol-2-yl)benzyl)-2-(5-fluoro-2-isopropylphenyl)-6,7-dimethyl-7,9-dihydro-8H-purin-8-imine (A-27d)

A stirred solution of compound A-27c (300 mg, 0.7 mmol) and 2-(5-fluoro-2-isopropylphenyl)-4,4,5,5-tetramethyl-1,3,2-dioxaborolane (322 mg, 1.0 mmol) in 1,4-dioxane (6 mL) and water (2 mL) was degassed by bubbling with argon for 10 minutes, then sodium carbonate (175 mg, 1.6 mmol) and Pd(dppf)Cl₂·DCM (53 mg, 0.07 mmol) were added and the reaction mixture was stirred at 90° C. for 16 h. The reaction mixture was diluted with water, filtered through the celite bed. The filtrate was extracted with EtOAc. The combined organic layer was washed with brine, dried (Na₂SO₄), filtered and concentrated under reduced pressure. The afforded crude compound was purified by column chromatography on silica gel, eluted with 5-10% MeOH/DCM. The residue was further purified by prep HPLC on Kromosil C18 (25×150) mm 10μ column using a gradient of 10 mM NH₄HCO₃ in H₂O: MeCN as mobile phase, which gave the title compound (55 mg, 16%) as a solid. LCMS (ES+) m/z 504.58 [M+H]⁺.

¹H NMR (500 MHz, DMSO): δ 7.62 (d, J=8.2 Hz, 2H), 7.42 (q, J=4.8 Hz, 3H), 7.35 (s, 1H), 7.27 (q, J=4.3 Hz, 1H), 7.19 (m, J=4.0 Hz, 1H), 6.42 (s, 1H), 5.16 (s, 2H), 3.68 (s, 3H), 3.56 (s, 3H), 3.46 (t, J=6.9 Hz, 1H), 2.67 (s, 3H), 1.07 (d, J=6.9 Hz, 6H).

### Example A-28

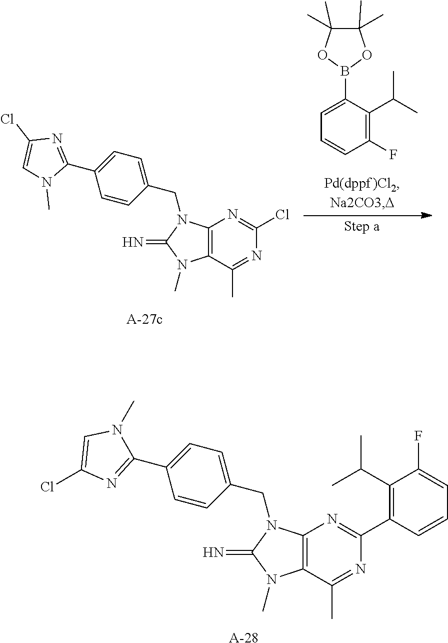

### 9-(4-(4-chloro-1-methyl-1H-imidazol-2-yl)benzyl)-2-(3-fluoro-2-isopropylphenyl)-6,7-dimethyl-7,9-dihydro-8H-purin-8-imine (A-28)

A stirred solution of compound A-27c (200 mg, 0.41 mmol) and 2-(3-fluoro-2-isopropylphenyl)-4,4,5,5-tetramethyl-1,3,2-dioxaborolane (205 mg, 0.62 mmol) in 1,4-dioxane (6 mL) and water (2 mL) was degassed by bubbling with argon for 10 minutes, then sodium carbonate (110 mg, 1.03 mmol) and Pd(dppf)Cl₂·DCM (34 mg, 0.04 mmol) were added and the reaction mixture was stirred at 90° C. for 16 h. The reaction mixture was diluted with water, filtered through the celite bed. The filtrate was extracted with EtOAc. The combined organic layer was washed with brine, dried (Na₂SO₄), filtered and concentrated under reduced pressure. The afforded crude compound was purified by column chromatography on silica gel, eluted with 5-10% MeOH/DCM. The residue was further purified by prep HPLC on Kromosil C18 (25×150) mm 10μ column using a gradient of 10 mM NH₄HCO₃ in H₂O: MeCN as mobile phase, which gave the title compound (35 mg, 16%) as a solid. LCMS (ES+) m/z 504.58 [M+H]⁺.

¹H NMR (500 MHz, DMSO): δ 7.62 (d, J=8.0 Hz, 2H), 7.42 (s, 2H), 7.35 (s, 1H), 7.27 (m, J=3.4 Hz, 2H), 7.16 (m, J=3.1 Hz, 1H), 6.38 (s, 1H), 5.15 (d, J=36.0 Hz, 2H), 3.68 (s, 3H), 3.56 (s, 3H), 3.28 (d, J=7.1 Hz, 1H), 2.67 (s, 3H), 1.19 (q, J=2.6 Hz, 6H).

### Example A-29

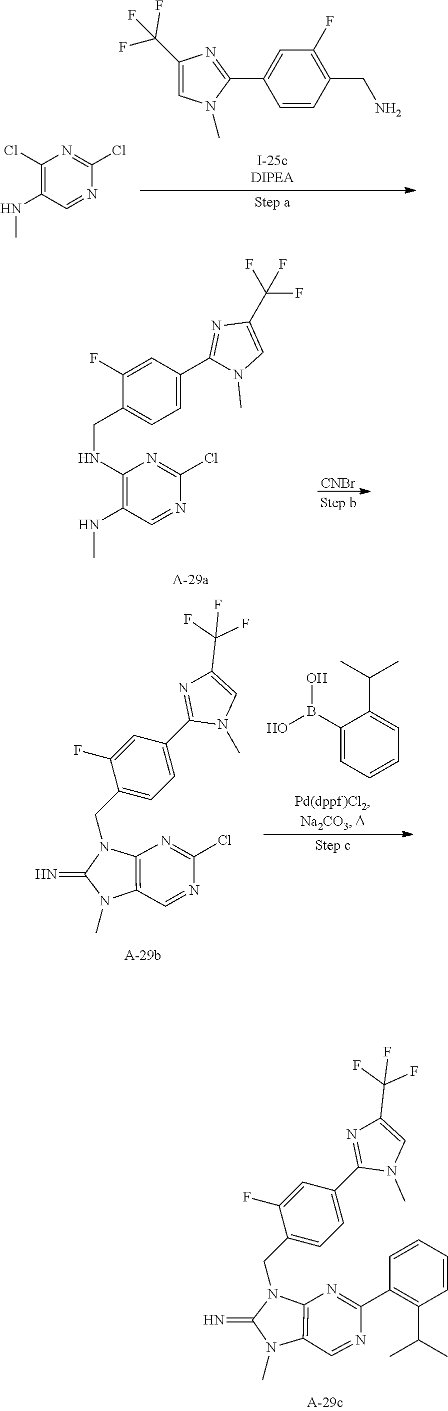

### Step a) 2-chloro-N4-(2-fluoro-4-(1-methyl-4-(trifluoromethyl)-1H-imidazol-2-yl)benzyl)-N₅-methylpyrimidine-4,5-diamine (A-29a)

DIPEA (730 mg, 5.62 mmol) and compound I-25c (530 mg, 1.1 mmol) were added to a stirred solution of 2,4-dichloro-N-methylpyrimidin-5-amine (200 mg, 1.1 mmol) in THE (20 mL), then was stirred for 40 h at 85° C., then concentrated under reduced pressure. The afforded residue was purified by column chromatography on silica gel, eluted with 30% EtOAc in pet ether, which gave the title compound (300 mg, 51%) as a solid. LCMS (ES+) 415.34 [M+H]⁺.

### Step b) 2-chloro-9-(2-fluoro-4-(1-methyl-4-(trifluoromethyl)-H-imidazol-2-yl)benzyl)-7-methyl-7,9-dihydro-8H-purin-8-imine (A-29b)

Cyanogen bromide (320 mg, 3.0 mmol) was added at 0° C. to a stirred solution of compound A-29a (250 mg, 0.63 mmol) in EtOH (20 mL). The resulting mixture was stirred at 85° C. for 24 h, then concentrated under reduced pressure, which gave the title compound (250 mg, 31%) as a solid. LCMS (ES+) 440.30 [M+H]⁺.

### Step c) 9-(2-fluoro-4-(1-methyl-4-(trifluoromethyl)-1H-imidazol-2-yl)benzyl)-2-(2-isopropylphenyl)-7-methyl-7,9-dihydro-8H-purin-8-imine (A-29c)

A stirred solution of compound A-29b (240 mg, 0.6 mmol), (2-isopropylphenyl) boronic acid (110 mg, 0.7 mmol) and sodium carbonate (121 mg, 1.2 mmol) in 1,4-dioxane (10 mL) and water (2 mL) was degassed by bubbling with argon for 10 minutes. Pd(dppf)Cl₂·DCM (22 mg, 0.03 mmol) was added and the reaction mixture was degassed by bubbling with argon for 10 minutes. The resulting reaction mixture was stirred at 120° C. for 2 h in microwave. The reaction mixture was filtered through the celite bed and the filtrate was concentrated under reduced pressure. The afforded crude compound was purified twice by column chromatography on silica gel, eluted with 5% MeOH/DCM. The residue was further purified by prep HPLC on Kromosil C₁₈ (25×150) mm 10μ column using a gradient of 10 mM NH₄HCO₃ in H₂O: MeCN as mobile phase and lyophilised, which gave the title compound (13 mg, 4%) as a solid. LCMS (ES+) m/z 524.57 [M+H]⁺.

¹H NMR (500 MHz, DMSO): δ 8.25 (s, 1H), 7.95 (d, J=1.1 Hz, 1H), 7.59 (d, J=11.2 Hz, 1H), 7.48 (m, J=3.4 Hz, 2H), 7.36 (m, J=3.4 Hz, 3H), 7.21 (m, J=3.2 Hz, 1H), 6.52 (s, 1H), 5.20 (s, 2H), 3.78 (s, 3H), 3.40 (s, 4H), 1.05 (d, J=6.8 Hz, 6H).

### Example A-30

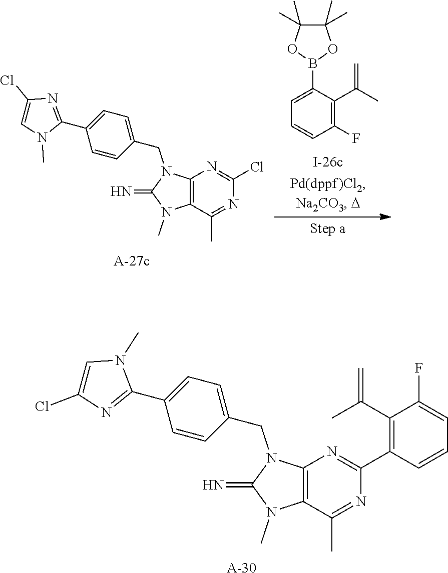 [SMILES: Cc1nc(Cl)nc2c1n(C)c(=N)n2Cc1ccc(-c2nc(Cl)cn2C)cc1 ; C=C(C)c1c(F)cccc1B1OC(C)(C)C(C)(C)O1 ; C=C(C)c1c(F)cccc1-c1nc(C)c2c(n1)n(Cc1ccc(-c3nc(Cl)cn3C)cc1)c(=N)n2C ; "A-30"]

### 9-(4-(4-chloro-1-methyl-1H-imidazol-2-yl)benzyl)-2-(3-fluoro-2-(prop-1-en-2-yl)phenyl)-6,7-dimethyl-7,9-dihydro-8H-purin-8-imine (A-30)

A stirred solution of compound A-27c (200 mg, 0.41 mmol) and compound I-26c (282 mg, 0.54 mmol) in 1,4-dioxane (6 mL) and water (2 mL) was degassed by bubbling with argon for 10 minutes, then sodium carbonate (110 mg, 1.03 mmol) and Pd(dppf)Cl₂·DCM (34 mg, 0.04 mmol) were added and the reaction mixture was stirred at 90° C. for 16 h. The reaction mixture was diluted with water, filtered through the celite bed. The filtrate was extracted with EtOAc. The combined organic layer was washed with brine, dried (Na₂SO₄), filtered and concentrated under reduced pressure. The afforded crude compound was purified by column chromatography on silica gel, eluted with 5-10% MeOH/DCM. The residue was further purified by prep HPLC on Kromosil C18 (25×150) mm 10μ column using a gradient of 10 mM NH₄HCO₃ in H₂O: MeCN as mobile phase, which gave the title compound (20 mg, 9%) as a solid. LCMS (ES+) m/z 502.56 [M+H]⁺.

¹H NMR (500 MHz, DMSO): δ 7.63 (d, J=8.3 Hz, 2H), 7.55 (q, J=2.9 Hz, 1H), 7.38 (m, J=5.2 Hz, 4H), 7.23 (m, J=4.8 Hz, 1H), 6.45 (s, 1H), 5.15 (s, 2H), 4.88 (s, 1H), 4.47 (s, 1H), 3.69 (s, 3H), 3.55 (s, 3H), 2.66 (s, 3H), 1.98 (s, 3H).

### Example A-3

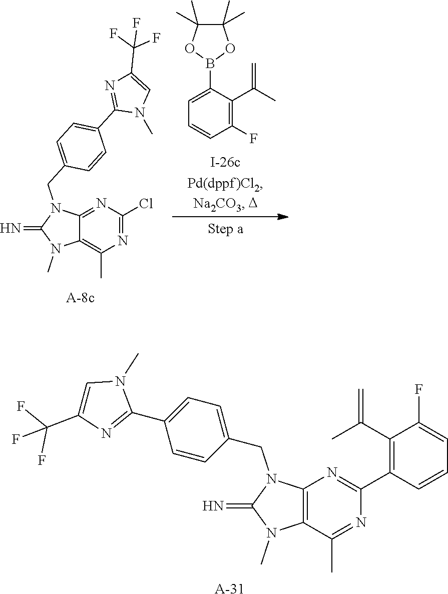

### 2-(3-fluoro-2-(prop-1-en-2-yl)phenyl)-6,7-dimethyl-9-(4-(1-methyl-4-(trifluoromethyl)-1H-imidazol-2-yl)benzyl)-7,9-dihydro-8H-purin-8-imine (A-31)

A stirred solution of compound A-8c (130 mg, 0.3 mmol) and compound I-26c (190 mg, 0.4 mmol) in 1,4-dioxane (12 mL) and water (3 mL) was degassed by bubbling with argon for 10 minutes, then sodium carbonate (74 mg, 0.7 mmol) and Pd(dppf)Cl₂·DCM (23 mg, 0.03 mmol) were added and the reaction mixture was stirred at 90° C. for 16 h. The reaction mixture was diluted with water, filtered through the celite bed. The filtrate was extracted with EtOAc. The combined organic layer was washed with brine, dried (Na₂SO₄), filtered and concentrated under reduced pressure. The afforded crude compound was purified by column chromatography on silica gel, eluted with 5-10% MeOH/DCM. The residue was further purified twice by prep HPLC on Kromosil C18 (25×150) mm 10μ column using a gradient of 10 mM NH₄HCO₃ in H₂O: MeCN as mobile phase, which gave the title compound (30 mg, 19%) as a solid. LCMS (ES+) m/z 536.60 [M+H]⁺.

¹H NMR (500 MHz, DMSO): δ 7.92 (d, J=1.0 Hz, 1H), 7.68 (d, J=8.2 Hz, 2H), 7.56 (q, J=2.9 Hz, 1H), 7.43 (d, J=8.3 Hz, 2H), 7.38 (m, J=4.3 Hz, 1H), 7.23 (m, J=3.8 Hz, 1H), 6.34 (s, 1H), 5.15 (s, 2H), 4.89 (t, J=1.6 Hz, 1H), 4.47 (d, J=0.8 Hz, 1H), 3.75 (s, 3H), 3.54 (s, 3H), 2.66 (s, 3H), 1.99 (s, 3H).

### Example A-32

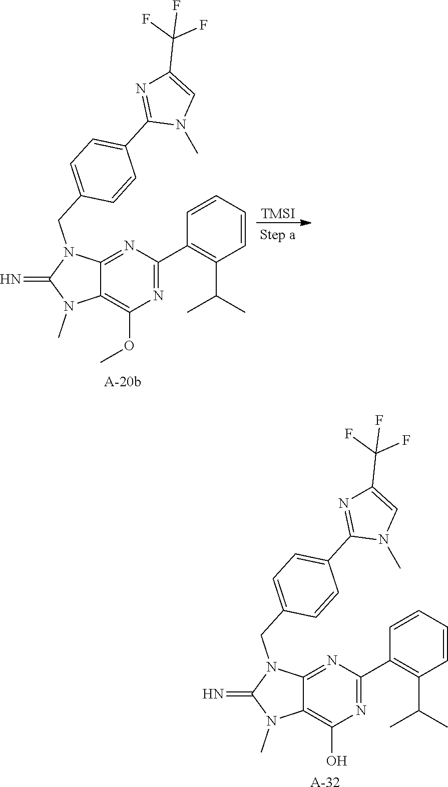

### 8-imino-2-(2-isopropylphenyl)-7-methyl-9-(4-(1-methyl-4-(trifluoromethyl)-1H-imidazol-2-yl)benzyl)-8,9-dihydro-7H-purin-6-ol (A-32)

Trimethyliodosilane (0.31 mL, 2.2 mmol) was added to a stirred solution of compound A-20b (300 mg, 0.6 mmol) in acetonitrile (5 mL) at 0° C. The resulting mixture was stirred for 6 h at 80° C., then concentrated under reduced pressure. Ice water was added and the mixture was extracted with EtOAc. The organic layer was washed with dried (Na₂SO₄), filtered and concentrated. The crude compound was purified by column chromatography on silica gel, eluted with 9% MeOH in DCM, which gave the title compound (4.5 g) as a solid. The impure compound was purified twice by prep HPLC on a Kromosil C18 (25×150) mm 10μ column using a gradient of 10 mM NH₄HCO₃IN H₂O: MeCN as mobile phase, which gave the title compound (34 mg, 11%) as a solid. LCMS (ES+) 522.59 [M+H]⁺.

¹H NMR (500 MHz, DMSO): δ 8.28 (s, 1H), 7.92 (d, J=1.0 Hz, 1H), 7.68 (d, J=8.3 Hz, 2H), 7.44 (d, J=8.3 Hz, 2H), 7.33 (t, J=3.6 Hz, 3H), 7.18 (d, J=5.0 Hz, 1H), 5.25 (s, 2H), 3.75 (s, 6H), 1.05 (d, J=6.9 Hz, 6H).

### Example A-33

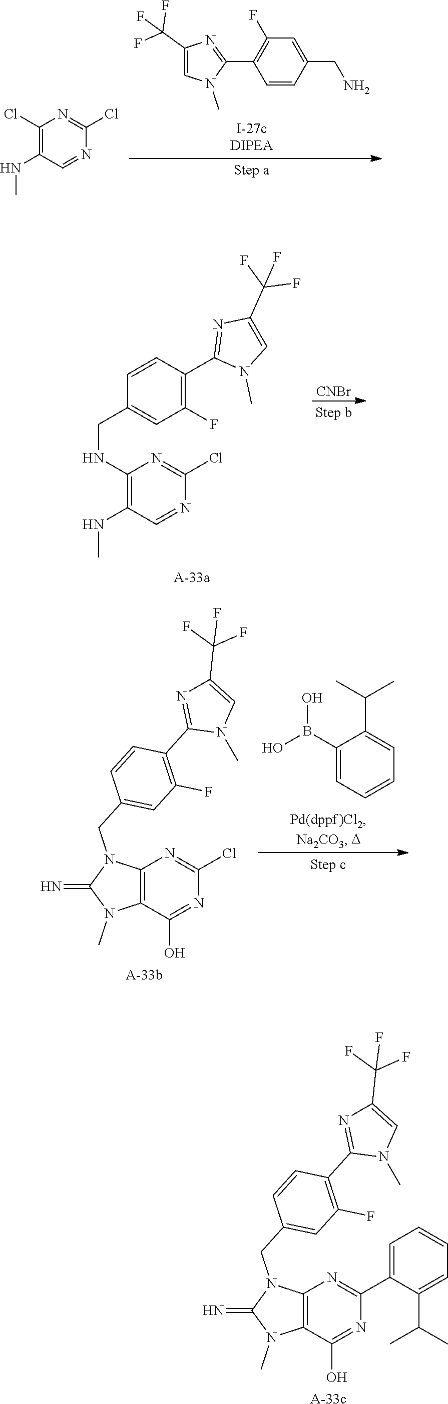

### Step a) 2-chloro-N4-(3-fluoro-4-(1-methyl-4-(trifluoromethyl)-1H-imidazol-2-yl)benzyl)-N₅-methylpyrimidine-4,5-diamine (A-33a)

DIPEA (360 mg, 2.8 mmol) was added to a stirred solution of 2,4-dichloro-N-methylpyrimidin-5-amine (100 mg, 0.6 mmol) and compound I-27c (385 g, 0.6 mmol) in THE (10 mL) at 0° C. and the resulting mixture was stirred for 48 h at 85° C., then concentrated under reduced pressure. The afforded residue was purified by column chromatography on silica gel, eluted with 50% EtOAc in pet ether, which gave the title compound (200 mg, 61%). LCMS (ES+) 415.28 [M+H]⁺.

### Step b) 2-chloro-9-(3-fluoro-4-(1-methyl-4-(trifluoromethyl)-H-imidazol-2-yl)benzyl)-7-methyl-7,9-dihydro-8H-purin-8-imine (A-33b)

Cyanogen bromide (192 mg, 1.8 mmol) was added at 0° C. to a stirred solution of compound A-33a (150 mg, 0.4 mmol) in EtOH (10 mL). The resulting mixture was stirred at 90° C. for 40 h, then concentrated under reduced pressure. The obtained residue was triturated with pentane, diethyl ether and EtOAc, which gave the title compound (159 mg, 25%). LCMS (ES+) 440.27 [M+H]⁺.

### Step c)

### 9-(3-fluoro-4-(1-methyl-4-(trifluoromethyl)-1H-imidazol-2-yl)benzyl)-2-(2-isopropylphenyl)-7-methyl-7,9-dihydro-8H-purin-8-imine (A-33c)

A stirred solution of compound A-33b (100 mg, 0.23 mmol), (2-isopropylphenyl) boronic acid (45 mg, 0.3 mmol) and sodium carbonate (51 mg, 0.5 mmol) in 1,4-dioxane (5 mL) and water (1 mL) in a microwave vial was degassed by bubbling with argon for 10 minutes. Pd(dppf)Cl₂·DCM (9 mg, 0.01 mmol) was added and the reaction mixture was degassed by bubbling with argon for 10 minutes. The resulting reaction mixture was stirred at 120° C. for 2 h in microwave. The reaction mixture was filtered through the celite bed and the filtrate was concentrated under reduced pressure. The residue was purified twice by prep HPLC on Kromosil C₁₈ (25×150) mm 10μ column using a gradient of 10 mM NH₄HCO₃ in H₂O: MeCN as mobile phase and lyophilised. The residue was further purified by SFC and lyophilised, which gave the title compound (8 mg, 6%). LCMS (ES+) m/z 524.61 [M+H]⁺.

¹H NMR (500 MHz, DMSO): δ 8.23 (s, 1H), 7.99 (d, J=1.0 Hz, 1H), 7.56 (t, J=7.7 Hz, 1H), 7.47 (q, J=3.0 Hz, 1H), 7.38 (m, J=3.4 Hz, 3H), 7.31 (d, J=6.1 Hz, 1H), 7.22 (m, J=3.2 Hz, 1H), 6.52 (d, J=4.7 Hz, 1H), 5.19 (q, J=13.6 Hz, 2H), 3.57 (d, J=1.2 Hz, 3H), 3.42 (q, J=8.0 Hz, 4H), 1.08 (d, J=6.9 Hz, 6H).

Preparative SFC Conditions
Column/dimensions: Chiralcel OJ-H (250×21 mm), 5p
CO₂: 90.0%
Co-solvent: 10.0% (0.5% diethylamine in methanol)
Total flow: 70.0 g/min
Back pressure: 90.0 bar
UV: 214 nm
Stack time: 14 min
Load/Inj.: 1.5 mg

### Example A-34

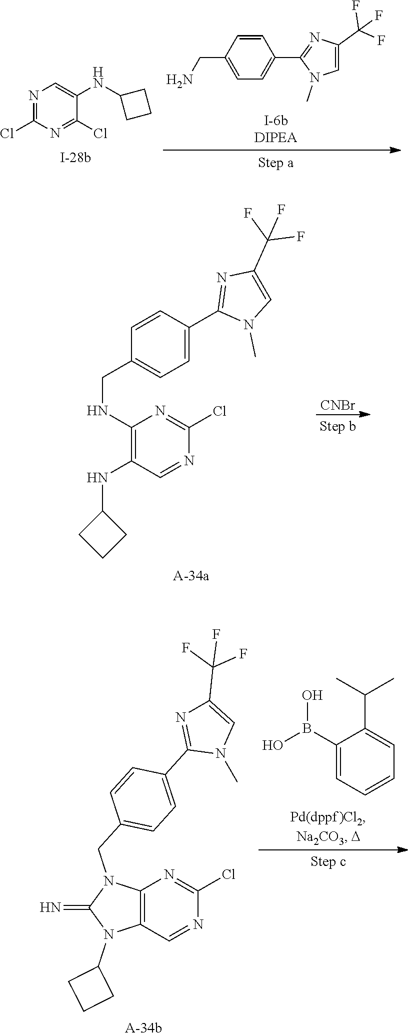

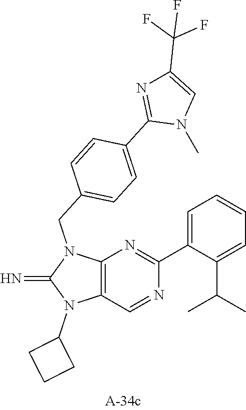 [SMILES: CC(C)c1ccccc1-c1ncc2c(n1)n(Cc1ccc(-c3nc(C(F)(F)F)cn3C)cc1)c(=N)n2C1CCC1]

### Step a) 2-chloro-N₅-cyclobutyl-N4-(4-(1-methyl-4-(trifluoromethyl)-1H-imidazol-2-yl)benzyl)pyrimidine-4,5-diamine (A-34a)

DIPEA (8 mL, 45.8 mmol) was added to a stirred solution of compound I-28b (5.2 g, 15.3 mmol) and compound I-6b (4.1 g, 15.1 mmol) in THF (50 mL) at 0° C. and the resulting mixture was stirred for 24 h at 80° C., then concentrated under reduced pressure. The afforded residue was purified by column chromatography on silica gel, eluted with 30-40% EtOAc in pet ether, which gave the title compound (120 mg, 1%) as a solid.

### Step b) 2-chloro-7-cyclobutyl-9-(4-(1-methyl-4-(trifluoromethyl)-1H-imidazol-2-yl)benzyl)-7,9-dihydro-8H-purin-8-imine (A-34b)

Cyanogen bromide (42 mg, 0.4 mmol) was added at 0° C. to a stirred solution of compound A-34a (100 mg, 0.2 mmol) in EtOH (3 mL). The resulting mixture was stirred at 80° C. for 10 h, then concentrated under reduced pressure. The afforded residue was purified by column chromatography on silica gel, eluted with 80% EtOAc in pet ether, which gave the title compound (110 mg, 38%) as a solid. LCMS (ES+) 462.38 [M+H]⁺.

### Step c) 7-cyclobutyl-2-(2-isopropylphenyl)-9-(4-(1-methyl-4-(trifluoromethyl)-1H-imidazol-2-yl)benzyl)-7,9-dihydro-8H-purin-8-imine (A-34c)

To a stirred solution of compound A-34b (110 mg, 0.1 mmol) and (2-isopropylphenyl)boronic acid (17 mg, 0.1 mmol) in 1,4-dioxane (1.5 mL) and water (0.5 mL) was added sodium carbonate (21 mg, 0.2 mmol) in a microwave vial. The reaction mixture was degassed by bubbling with argon for 15 minutes, then Pd(dppf)Cl₂·DCM (6 mg, 0.01 mmol) was added and degassed by bubbling with argon for 5 minutes, then the reaction mixture was stirred at 100° C. for 1 h in a microwave. The reaction mixture was filtered through the celite bed and washed with EtOAc. The combined organic layer was, dried (Na₂SO₄), filtered and concentrated under reduced pressure. The residue was purified by prep HPLC on Kromosil C18 (25×150) mm 10μ column using a gradient of 10 mM NH₄HCO₃ in H₂O: MeCN as mobile phase, which gave the title compound (18 mg, 41%) as a solid. LCMS (ES+) m/z 546.65 [M+H]⁺.

¹H NMR (500 MHz, DMSO): δ 8.53 (s, 1H), 7.91 (d, J=1.1 Hz, 1H), 7.68 (d, J=8.2 Hz, 2H), 7.49 (m, J=5.7 Hz, 3H), 7.38 (m, J=3.6 Hz, 2H), 7.23 (m, J=3.2 Hz, 1H), 6.57 (d, J=88.4 Hz, 1H), 5.05 (m, J=29.3 Hz, 3H), 3.75 (s, 3H), 3.48 (m, J=6.6 Hz, 1H), 2.76 (t, J=5.5 Hz, 2H), 2.34 (m, J=5.2 Hz, 2H), 1.92 (t, J=6.4 Hz, 1H), 1.77 (m, J=4.5 Hz, 1H), 1.10 (d, J=6.9 Hz, 6H).

### Example A-35

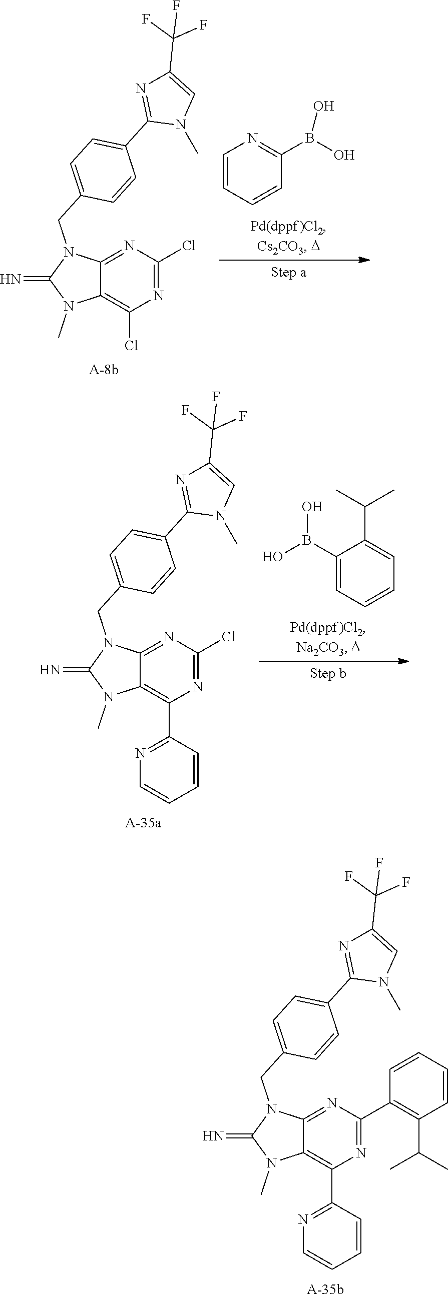

### Step a) 2-chloro-7-methyl-9-(4-(1-methyl-4-(trifluoromethyl)-1H-imidazol-2-yl)benzyl)-6-(pyridin-2-yl)-7,9-dihydro-8H-purin-8-imine (A-35a)

Pyridin-2-ylboronic acid (317 mg, 2.6 mmol) and cesium carbonate (3.5 g, 11 mmol) were added to a stirred solution of compound A-8b (1.0 g, 2.2 mmol) in 1,4-dioxane (24 mL) and water (6 mL) in a sealed tube. The reaction mixture was degassed by bubbling with argon for 15 minutes then Pd(dppf)Cl₂·DCM (176 mg, 0.22 mmol) was added and the reaction mixture was degassed by bubbling with argon for 5 minutes and stirred at 100° C. for 16 h in a sealed tube. The reaction mixture was filtered through a celite bed. The filtrate was concentrated under reduced pressure. The afforded crude compound was purified by column chromatography on silica gel, eluted with 7% MeOH/DCM, which gave the title compound (220 mg, 5%) as a solid.

LCMS (ES+) m/z 499.45 [M+H]⁺.

### Step b) 2-(2-isopropylphenyl)-7-methyl-9-(4-(1-methyl-4-(trifluoromethyl)-1H-imidazol-2-yl)benzyl)-6-(pyridin-2-yl)-7,9-dihydro-8H-purin-8-imine (A-35b)

(2-Isopropylphenyl)boronic acid (40 mg, 0.24 mmol) and sodium carbonate (38 mg, 0.4 mmol) were added to a stirred solution of compound A-35a (220 mg, 0.12 mmol) in 1,4-dioxane (9 mL) and water (3 mL) in a sealed tube. The reaction mixture was degassed by bubbling with argon for 15 min then Pd(dppf)Cl₂·DCM (10 mg, 0.01 mmol) was added and the mixture was degassed for 5 minutes, then stirred at 100° C. for 16 h in a sealed tube. The reaction mixture was filtered through the celite bed and the filtrate was concentrated under reduced pressure. The afforded crude compound was purified by column chromatography on silica gel, eluted with 7% MeOH/DCM. The residue was purified by prep HPLC on Kromosil C18 (25×150) mm 10p column using a gradient of 10 mM NH₄HCO₃ in H₂O: MeCN as mobile phase, which gave the title compound (40 mg, 57%) as a solid. MS (ES+) m/z 583.69 [M+H]⁺.

¹H NMR (500 MHz, DMSO): δ 8.75 (d, J=4.1 Hz, 1H), 8.09 (d, J=7.9 Hz, 1H), 8.01 (m, J=3.4 Hz, 1H), 7.92 (d, J=0.9 Hz, 1H), 7.70 (d, J=8.2 Hz, 2H), 7.61 (q, J=2.9 Hz, 1H), 7.51 (m, J=2.1 Hz, 3H), 7.41 (m, J=4.8 Hz, 2H), 7.25 (m, J=3.2 Hz, 1H), 6.77 (s, 1H), 5.27 (s, 2H), 3.75 (s, 3H), 3.55 (m, J=6.8 Hz, 1H), 3.37 (s, 3H), 1.14 (d, J=6.9 Hz, 6H).

### Example A-36

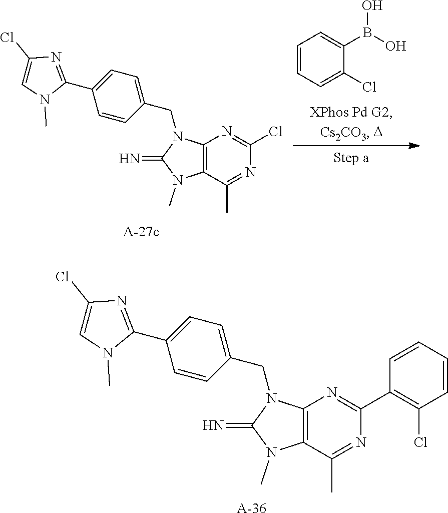

### 9-(4-(4-chloro-1-methyl-1H-imidazol-2-yl)benzyl)-2-(2-chlorophenyl)-6,7-dimethyl-7,9-dihydro-8H-purin-8-imine (A-36)

(2-Chlorophenyl)boronic acid (158 mg, 1 mmol) and cesium carbonate (820 mg, 2.5 mmol) were added to a stirred solution of compound A-27c (250 mg, 0.5 mmol) in 1,4-dioxane (15 mL) in a sealed tube. The reaction mixture was degassed by bubbling with argon for 20 min, then XPhos-Pd-G2 (40 mg, 0.05 mmol) was added and the mixture was degassed for 10 minutes, then stirred at 110° C. for 16 h in a sealed tube. The reaction mixture was filtered through the celite bed, washed with EtOAc (100 mL) and the filtrate was concentrated under reduced pressure. The afforded crude compound was purified by column chromatography on silica gel, eluted with 5% MeOH/DCM. The residue was purified by prep HPLC on Kromosil C18 (25×150) mm 10μ column using a gradient of 10 mM NH₄HCO₃ in H₂O: MeCN as mobile phase and lyophilised, which gave the title compound (17 mg, 6%) as a solid. MS (ES+) m/z 478.41 [M+H]⁺.

¹H NMR (500 MHz, DMSO): δ 7.63 (m, J=4.2 Hz, 3H), 7.46 (m, J=4.4 Hz, 5H), 7.35 (s, 1H), 6.39 (d, J=17.7 Hz, 1H), 5.15 (d, J=48.2 Hz, 2H), 3.68 (s, 3H), 3.55 (s, 3H), 2.67 (d, J=8.8 Hz, 3H).

### Example A-37

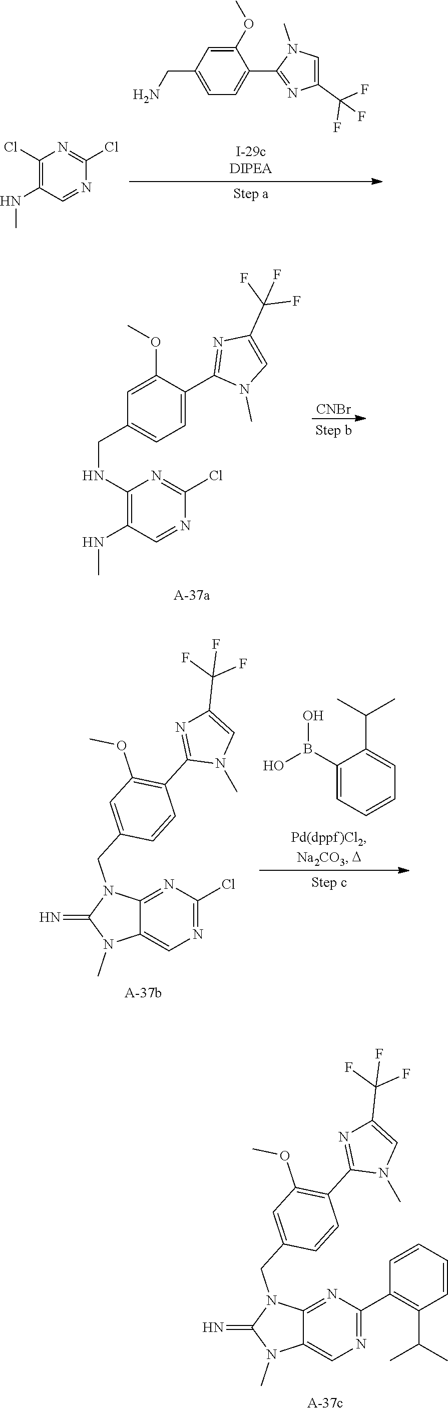

### Step a) 2-chloro-N4-(3-methoxy-4-(1-methyl-4-(trifluoromethyl)-1H-imidazol-2-yl)benzyl)-N₅-methylpyrimidine-4,5-diamine (A-37a)

DIPEA (2.2 mL, 12.6 mmol) was added to a stirred solution of compound I-29c (1.8 g, 5.9 mmol) and 2,4-dichloro-N-methylpyrimidin-5-amine (1.12 g, 6.2 mmol) in THE (30 mL) at 0° C. and the resulting mixture was stirred for 24 h at 80° C., then concentrated under reduced pressure. The afforded residue was purified by column chromatography on silica gel, eluted with 80-90% EtOAc in pet ether, which gave the title compound (1.3 g, 48%) as a solid. LCMS (ES+) 427.27 [M+H]⁺.

### Step b) 2-chloro-9-(3-methoxy-4-(1-methyl-4-(trifluoromethyl)-1H-imidazol-2-yl)benzyl)-7-methyl-7,9-dihydro-8H-purin-8-imine (A-37b)

Cyanogen bromide (60 mg, 0.6 mmol) was added at 0° C. to a stirred solution of compound A-37a (100 mg, 0.22 mmol) in EtOH (5 mL). The resulting mixture was stirred at 80° C. for 6 h, then concentrated under reduced pressure. The obtained residue was triturated with diethyl ether, which gave the title compound (130 mg, 83%). LCMS (ES+) 452.27 [M+H]⁺.

### Step c) 2-(2-isopropylphenyl)-9-(3-methoxy-4-(1-methyl-4-(trifluoromethyl)-1H-imidazol-2-yl)benzyl)-7-methyl-7,9-dihydro-8H-purin-8-imine (A-37c)

To a stirred solution of compound A-37b (130 mg, 0.2 mmol) and (2-isopropylphenyl)boronic acid (40 mg, 0.24 mmol) in 1,4-dioxane (1.5 mL) and water (0.5 mL) was added sodium carbonate (50 mg, 0.5 mmol) in a microwave vial. The reaction mixture was degassed by bubbling with argon for 15 minutes, then Pd(dppf)Cl₂·DCM (15 mg, 0.02 mmol) was added and the mixture was degassed for 5 minutes, then stirred at 100° C. for 2 h in a microwave. The reaction mixture was diluted with EtOAc, filtered through the celite bed. The combined organic layer was, dried (Na₂SO₄), filtered and concentrated under reduced pressure. The residue was purified by prep HPLC on Kromosil C18 (25×150) mm 10μ column using a gradient of 10 mM NH₄HCO₃ in H₂O: MeCN as mobile phase, which gave the title compound (25 mg, 25%) as a solid. LCMS (ES+) m/z 536.60 [M+H]⁺.

¹H NMR (500 MHz, DMSO): δ 8.23 (s, 1H), 7.88 (d, J=1.1 Hz, 1H), 7.48 (q, J=3.0 Hz, 1H), 7.38 (m, J=3.5 Hz, 2H), 7.29 (t, J=7.8 Hz, 2H), 7.22 (m, J=3.2 Hz, 1H), 6.94 (d, J=7.8 Hz, 1H), 6.53 (s, 1H), 5.16 (s, 2H), 3.76 (s, 3H), 3.42 (d, J=31.8 Hz, 7H), 1.09 (d, J=6.9 Hz, 6H).

### Example A-38

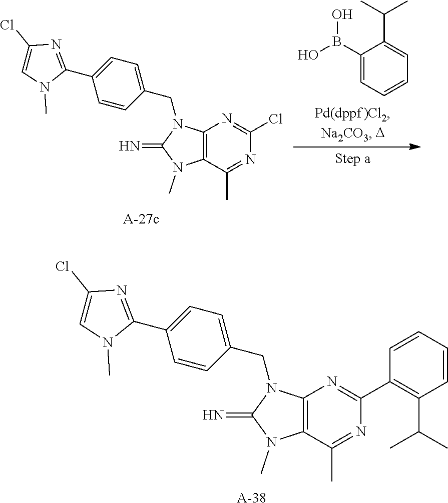

### Step a) 9-(4-(4-chloro-1-methyl-1H-imidazol-2-yl)benzyl)-2-(2-isopropylphenyl)-6,7-dimethyl-7,9-dihydro-8H-purin-8-imine (A-38)

(2-Isopropylphenyl)boronic acid (142 mg, 0.9 mmol) and sodium carbonate (300 mg, 2.8 mmol) were added to a stirred solution of compound A-27c (400 mg, 0.6 mmol) in 1,4-dioxane (8 mL) and water (2 mL). The reaction mixture was degassed by bubbling with argon for 15 min then Pd(dppf)Cl₂·DCM (47 mg, 0.06 mmol) was added and the mixture was degassed for 5 minutes, then stirred at 110° C. for 16 h. The reaction mixture was diluted with ice water, extracted with EtOAc (3×25 mL). The combined organic layers were washed with brine (15 mL), dried (Na₂SO₄), filtered and concentrated under reduced pressure. The residue was purified by prep HPLC on Kromosil C18 (25×150) mm 10μ column using a gradient of 10 mM NH₄HCO₃ in H₂O: MeCN as mobile phase and lyophilised, which gave the title compound (100 mg, 34%) as a solid. LCMS (ES+) m/z 486.57[M+H]⁺.

¹H NMR (500 MHz, DMSO): δ 7.62 (d, J=8.2 Hz, 2H), 7.48 (q, J=2.9 Hz, 1H), 7.42 (d, J=7.8 Hz, 2H), 7.36 (m, J=4.0 Hz, 3H), 7.21 (m, J=3.2 Hz, 1H), 6.36 (s, 1H), 5.15 (s, 1H), 3.68 (s, 3H), 3.56 (s, 3H), 3.42 (t, J=6.8 Hz, 1H), 2.67 (s, 3H), 1.09 (d, J=6.9 Hz, 6H).

### Example A-39

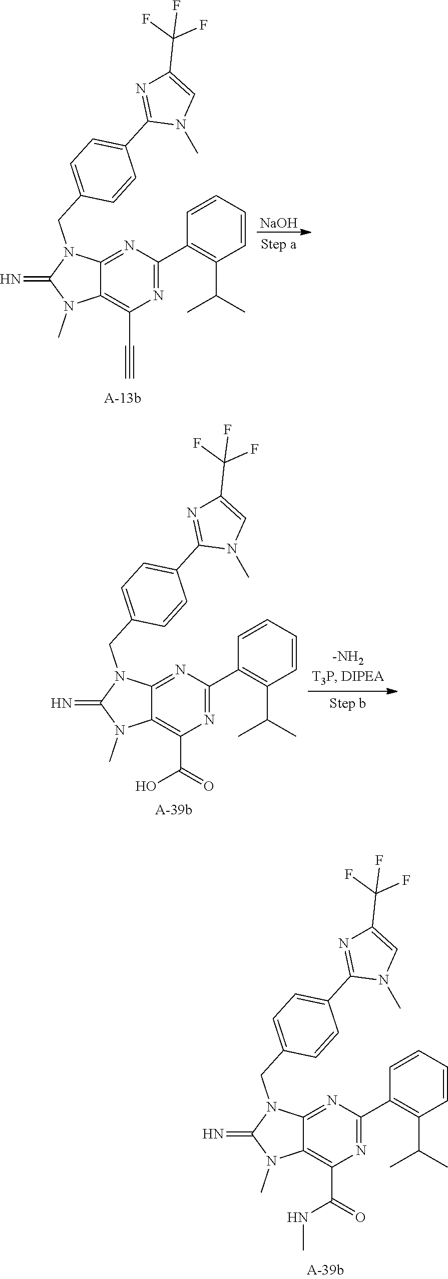

### Step a) 8-imino-2-(2-isopropylphenyl)-7-methyl-9-(4-(1-methyl-4-(trifluoromethyl)-1H-imidazol-2-yl)benzyl)-8,9-dihydro-7H-purine-6-carboxylic acid (A-39a)

To a stirred solution of compound A-13b (100 mg, 0.2 mmol) in MeOH (10 mL) and water (2 mL) was added NaOH (38 mg, 0.94 mmol) at rt. The resulting mixture was stirred at 60° C. for 16 h, then concentrated. Water was added and neutralised with 6N HCl. The precipitated solid was filtered and dried, which gave the title compound (90 mg, 86%) as a solid. LCMS (ES+) 550.61 [M+H]⁺.

### Step b) 8-imino-2-(2-isopropylphenyl)-N, 7-dimethyl-9-(4-(1-methyl-4-(trifluoromethyl)-1H-imidazol-2-yl)benzyl)-8,9-dihydro-7H-purine-6-carboxamide (A-39b)

DIPEA (57 mg, 0.43 mmol) and T₃P (113 mg, 0.43 mmol) were added at 0° C. solution of compound A-39a (80 mg, 0.15 mmol) in DCM (15 mL). The mixture was stirred for 10 min, then methylamine (2M in THF) (0.73 mL, 1.5 mmol) was added at 0° C. The mixture was stirred at rt for 16 h. Water was added and the mixture was extracted with DCM (3×20 mL). The organic layer was washed with water, brine and dried (Na₂SO₄), filtered and concentrated under reduced pressure. The residue was purified by prep HPLC on Kromosil C18 (25×150) mm 10μ column using a gradient of 10 mM NH₄HCO₃ in H₂O: MeCN as mobile phase and lyophilised, which gave the title compound (65 mg, 79%) as a solid. LCMS (ES+) m/z 563.62 [M+H]⁺.

¹H NMR (500 MHz, DMSO): δ 8.66 (q, J=4.6 Hz, 1H), 7.91 (d, J=1.2 Hz, 1H), 7.67 (t, J=4.2 Hz, 2H), 7.62 (q, J=2.9 Hz, 1H), 7.46 (d, J=8.3 Hz, 2H), 7.41 (m, J=3.9 Hz, 2H), 7.25 (m, J=2.3 Hz, 1H), 6.94 (s, 1H), 5.23 (s, 2H), 3.74 (s, 3H), 3.66 (s, 3H), 3.41 (d, J=5.7 Hz, 1H), 2.83 (d, J=4.9 Hz, 3H), 1.09 (d, J=6.9 Hz, 6H).

### Example A-40

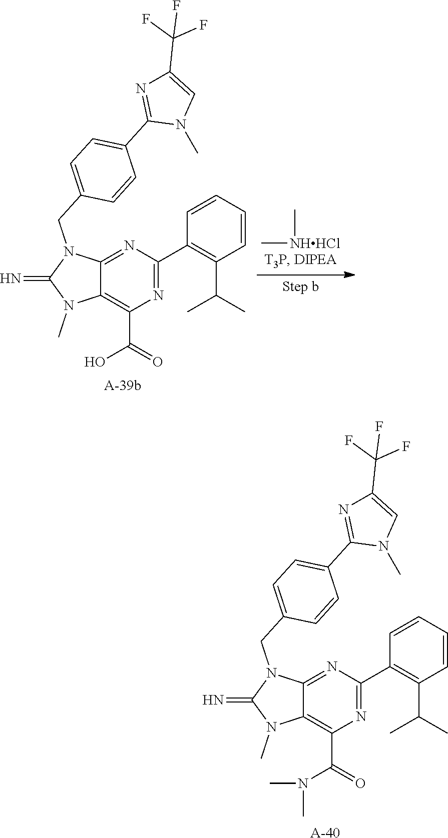

### Step a) 8-imino-2-(2-isopropylphenyl)-N,N, 7-trimethyl-9-(4-(1-methyl-4-(trifluoromethyl)-1H-imidazol-2-yl)benzyl)-8,9-dihydro-7H-purine-6-carboxamide (A-40)

DIPEA (0.07 mL, 0.4 mmol) and T₃P (0.1 mL, 0.4 mmol) were added at 0° C. solution of compound A-39a (70 mg, 0.13 mmol) in DCM (15 mL). The mixture was stirred for 10 min, then dimethylamine hydrochloride (21 mg, 0.3 mmol) was added at 0° C. The mixture was stirred at rt for 16 h. Water was added and the mixture was extracted with DCM (3×20 mL). The organic layer was washed with water, brine and dried (Na₂SO₄), filtered and concentrated under reduced pressure. The residue was combined with another batch and purified by prep HPLC on Kromosil C₁₈ (25×150) mm 10μ column using a gradient of 10 mM NH₄HCO₃ in H₂O: MeCN as mobile phase and lyophilised, which gave the title compound (60 mg) as a solid. LCMS (ES+) m/z 577.63 [M+H]⁺.

¹H NMR (500 MHz, DMSO): δ 7.92 (d, J=1.1 Hz, 1H), 7.68 (d, J=8.1 Hz, 2H), 7.49 (m, J=4.7 Hz, 3H), 7.39 (m, J=3.1 Hz, 2H), 7.24 (m, J=2.3 Hz, 1H), 6.76 (d, J=36.3 Hz, 1H), 5.20 (d, J=40.1 Hz, 2H), 3.75 (s, 3H), 3.43 (s, 1H), 3.28 (s, 3H), 3.06 (s, 3H), 3.01 (s, 3H), 1.09 (d, J=6.9 Hz, 6H).

### Example A-41

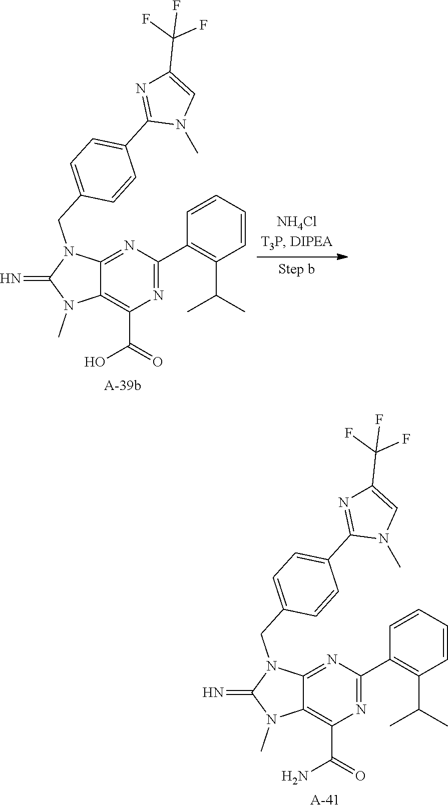

### Step a) 8-imino-2-(2-isopropylphenyl)-7-methyl-9-(4-(1-methyl-4-(trifluoromethyl)-1H-imidazol-2-yl)benzyl)-8,9-dihydro-7H-purine-6-carboxamide (A-41)

DIPEA (0.06 mL, 0.3 mmol) and T₃P (0.08 mL, 0.3 mmol) were added at 0° C. solution of compound A-39a (60 mg, 0.11 mmol) in DCM (15 mL). The mixture was stirred for 10 min, then ammonium chloride (18 mg, 0.3 mmol) was added at 0° C. The mixture was stirred at rt for 16 h.

Ice water was added and the mixture was extracted with DCM (3×20 mL). The organic layer was washed with water, brine and dried (Na₂SO₄), filtered and concentrated under reduced pressure. The residue was combined with another batch and purified by prep HPLC on Kromosil C₁₈ (25×150) mm 10μ column using a gradient of 10 mM NH₄HCO₃ in H₂O: MeCN as mobile phase and lyophilised, which gave the title compound (52 mg) as a solid. LCMS (ES+) m/z 549.60 [M+H]⁺.

¹H NMR (500 MHz, DMSO): δ 8.04 (s, 1H), 7.91 (s, 1H), 7.83 (s, 1H), 7.67 (d, J=6.4 Hz, 2H), 7.63 (q, J=2.9 Hz, 1H), 7.43 (m, J=6.7 Hz, 4H), 7.25 (m, J=3.3 Hz, 1H), 6.81 (d, J=48.1 Hz, 1H), 5.22 (d, J=52.0 Hz, 2H), 3.75 (s, 3H), 3.63 (s, 3H), 3.46 (s, 1H), 1.10 (s, 6H).

### Example A-42

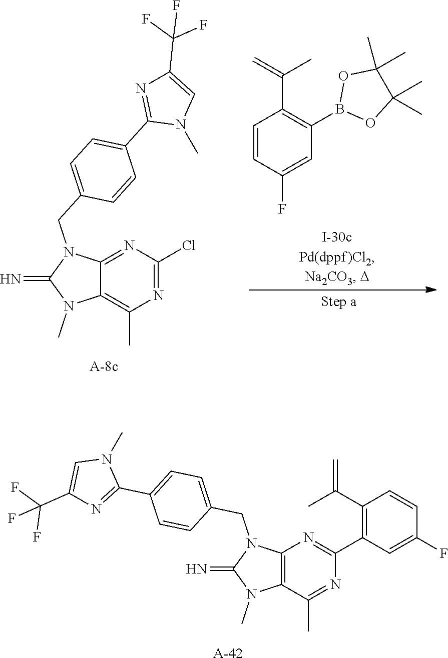

### Step a) 2-(5-fluoro-2-(prop-1-en-2-yl)phenyl)-6,7-dimethyl-9-(4-(1-methyl-4-(trifluoromethyl)-1H-imidazol-2-yl)benzyl)-7,9-dihydro-8H-purin-8-imine (A-42)

A stirred solution of compound A-8c (500 mg, 1.0 mmol) and compound I-30c (758 mg, 1.2 mmol) in 1,4-dioxane (10 mL) and water (3 mL) was degassed by bubbling with argon for 10 minutes, then sodium carbonate (255 mg, 2.4 mmol) and Pd(dppf)Cl₂·DCM (79 mg, 0.1 mmol) were added and the reaction mixture was stirred at 90° C. for 16 h. The reaction mixture was diluted with water, filtered through the celite bed. The filtrate was extracted with EtOAc. The combined organic layer was washed with brine, dried (Na₂SO₄), filtered and concentrated under reduced pressure. The afforded crude compound was purified by column chromatography on silica gel, eluted with 5-10% MeOH/DCM. The residue was further purified by prep HPLC on Kromosil C18 (25×150) mm 10μ column using a gradient of 10 mM NH₄HCO₃ in H₂O: MeCN as mobile phase, which gave the title compound (15 mg, 2%) as a solid. LCMS (ES+) m/z 536.60 [M+H]⁺.

¹H NMR (500 MHz, DMSO): δ 7.92 (d, J=0.9 Hz, 1H), 7.68 (d, J=8.0 Hz, 2H), 7.47 (m, J=3.9 Hz, 3H), 7.27 (q, J=4.8 Hz, 1H), 7.21 (m, J=3.9 Hz, 1H), 6.35 (s, 1H), 5.14 (d, J=42.0 Hz, 2H), 4.85 (s, 1H), 4.60 (s, 1H), 3.75 (s, 3H), 3.54 (s, 3H), 2.66 (s, 3H), 1.80 (s, 3H).

### Example A-43

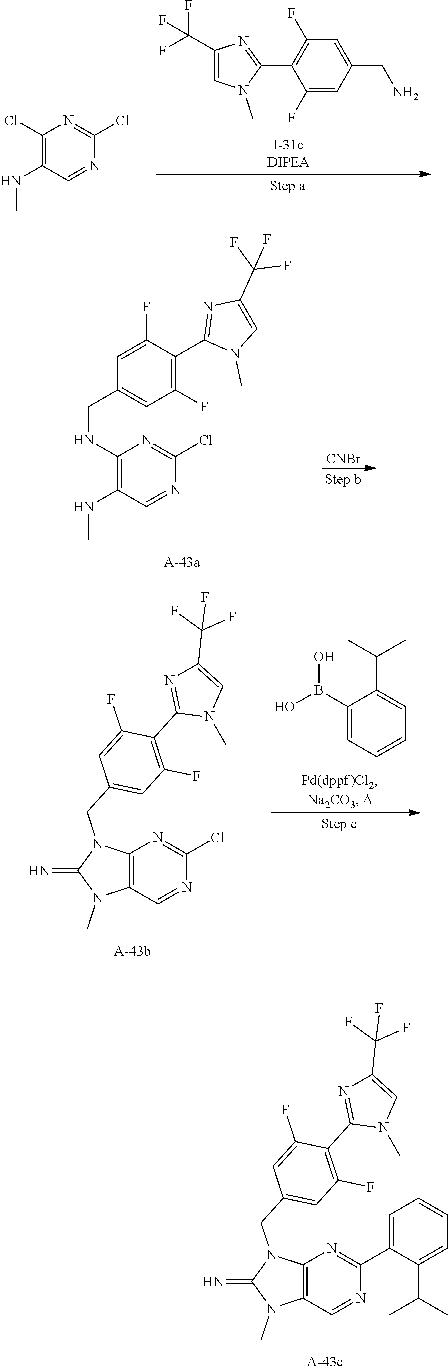

### Step a) 2-chloro-N4-(3,5-difluoro-4-(1-methyl-4-(trifluoromethyl)-1H-imidazol-2-yl)benzyl)-N₅-methylpyrimidine-4,5-diamine (A-43a)

DIPEA (1.5 g, 11.2 mmol) was added to a stirred solution of 2,4-dichloro-N-methylpyrimidin-5-amine (200 mg, 1.1 mmol) and compound I-31c (873 mg, 1.4 mmol) in THE (20 mL) at 0° C. and the resulting mixture was stirred for 48 h at 85° C., then concentrated under reduced pressure. The afforded residue was purified by column chromatography on silica gel, eluted with 50% EtOAc in pet ether, which gave the title compound (400 mg, 37%). LCMS (ES+) 433.32 [M+H]⁺.

### Step b) 2-chloro-9-(3,5-difluoro-4-(1-methyl-4-(trifluoromethyl)-1H-imidazol-2-yl)benzyl)-7-methyl-7,9-dihydro-8H-purin-8-imine (A-43b)

Cyanogen bromide (490 mg, 4.6 mmol) was added at 0° C. to a stirred solution of compound A-43a (400 mg, 0.92 mmol) in EtOH (10 mL). The resulting mixture was stirred at 95° C. for 24 h, then concentrated under reduced pressure, which gave the title compound (400 mg, 17%). LCMS (ES+) 458.33 [M+H]⁺.

### Step c) 9-(3,5-difluoro-4-(1-methyl-4-(trifluoromethyl)-1H-imidazol-2-yl)benzyl)-2-(2-isopropylphenyl)-7-methyl-7,9-dihydro-8H-purin-8-imine (A-43c)

A stirred solution of compound A-43b (350 mg, 0.8 mmol), (2-isopropylphenyl) boronic acid (150 mg, 0.9 mmol) and sodium carbonate (170 mg, 1.6 mmol) in 1,4-dioxane (5 mL) and water (1 mL) in a microwave vial was degassed by bubbling with argon for 10 minutes. Pd(dppf)Cl₂·DCM (62 mg, 0.08 mmol) was added and the reaction mixture was degassed by bubbling with argon for 10 minutes. The resulting reaction mixture was stirred at 120° C. for 2 h in microwave. The reaction mixture was filtered through the celite bed and the filtrate was concentrated under reduced pressure. The afforded crude compound was purified by column chromatography on silica gel, eluted with 5% MeOH/DCM. The obtained residue was further purified twice by reverse phase chromatography in H₂O: MeCN as mobile phase. The impure compound was further purified by prep HPLC on Kromosil C18 (25×150) mm 10μ column using a gradient of 10 mM NH₄HCO₃ in H₂O: MeCN as mobile phase and lyophilised, which gave the title compound (7 mg, 1%). LCMS (ES+) m/z 542.54 [M+H]⁺.

¹H NMR (500 MHz, DMSO): δ 8.24 (s, 1H), 8.06 (d, J=0.9 Hz, 1H), 7.47 (q, J=3.0 Hz, 1H), 7.38 (m, J=3.4 Hz, 2H), 7.29 (d, J=8.5 Hz, 2H), 7.22 (m, J=3.3 Hz, 1H), 6.52 (s, 1H), 5.20 (d, J=41.3 Hz, 2H), 3.55 (s, 3H), 3.42 (t, J=7.0 Hz, 3H), 1.08 (d, J=6.9 Hz, 6H).

### Example A-44

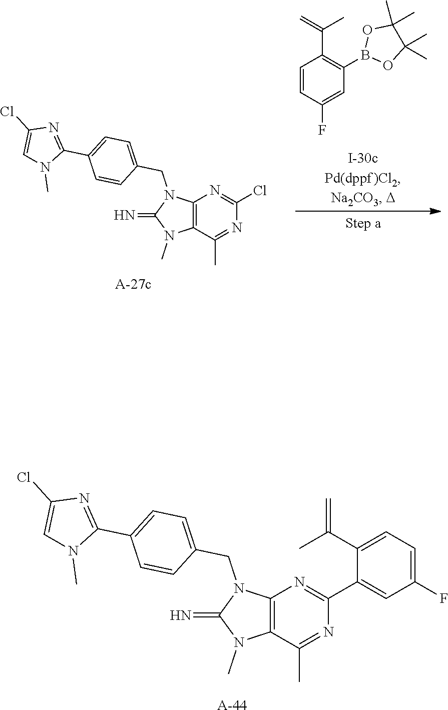

### Step a) 9-(4-(4-chloro-1-methyl-1H-imidazol-2-yl)benzyl)-2-(5-fluoro-2-(prop-1-en-2-yl)phenyl)-6,7-dimethyl-7,9-dihydro-8H-purin-8-imine (A-44)

A stirred solution of compound A-27c (300 mg, 0.7 mmol) and compound I-30c (553 mg, 0.8 mmol) in 1,4-dioxane (6 mL) and water (2 mL) was degassed by bubbling with argon for 10 minutes, then sodium carbonate (172 mg, 1.6 mmol) and Pd(dppf)Cl₂·DCM (53 mg, 0.07 mmol) were added and the reaction mixture was stirred at 90° C. for 16 h. The reaction mixture was diluted with water, filtered through the celite bed. The filtrate was extracted with EtOAc. The combined organic layer was washed with brine, dried (Na₂SO₄), filtered and concentrated under reduced pressure. The afforded crude compound was purified by column chromatography on silica gel, eluted with 5-10% MeOH/DCM. The residue was further purified by prep HPLC on Kromosil C18 (25×150) mm 10μ column using a gradient of 10 mM NH₄HCO₃ in H₂O: MeCN as mobile phase. The residue obtained from peak-1 was further purified by prep HPLC on Kromosil C18 (25×150) mm 10μ column using a gradient of 10 mM NH₄HCO₃ in H₂O: MeCN as mobile phase, which gave the title compound (20 mg, 6%) as a solid. LCMS (ES+) m/z 502.52 [M+H]⁺.

¹H NMR (500 MHz, DMSO): δ 7.63 (d, J=8.2 Hz, 2H), 7.45 (m, J=5.7 Hz, 3H), 7.35 (s, 1H), 7.27 (q, J=4.8 Hz, 1H), 7.21 (m, J=3.9 Hz, 1H), 6.36 (s, 1H), 5.12 (s, 1H), 4.84 (s, 1H), 4.59 (s, 1H), 3.69 (s, 3H), 3.54 (s, 3H), 2.66 (s, 3H), 1.79 (s, 3H).

### Example A-45

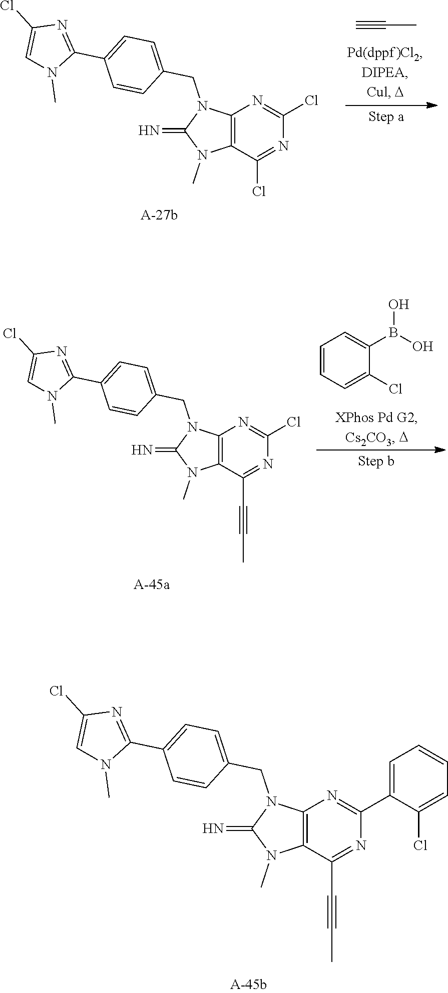

### Step a) 2-chloro-9-(4-(4-chloro-1-methyl-1H-imidazol-2-yl)benzyl)-7-methyl-6-(prop-1-yn-1-yl)-7,9-dihydro-8H-purin-8-imine (A-45a)

DIPEA (0.6 mL, 3.4 mmol), copper iodide (41 mg, 0.2 mmol) and Pd(dppf)Cl₂·DCM (74 mg, 0.1 mmol) were added at rt to a stirred solution of compound A-27b (500 mg, 1.0 mmol) in DMF (10 mL). The reaction mixture was degassed by bubbling with argon for 20 minutes, then prop-1-yne (0.05 mL, 1.1 mmol) was added and the reaction mixture was stirred at 100° C. for 16 h in a sealed tube. The reaction mixture was filtered through the celite bed and washed with EtOAc (50 mL). The filtrate was diluted with water (100 mL) and extracted with EtOAc (2×100 mL). The combined organic layer was dried (Na₂SO₄), filtered and concentrated under reduced pressure, which gave the title compound (600 mg, 58%) as a liquid. MS (ES+) m/z 426.40 [M+H]⁺.

### Step b) 9-(4-(4-chloro-1-methyl-1H-imidazol-2-yl)benzyl)-2-(2-chlorophenyl)-7-methyl-6-(prop-1-yn-1-yl)-7,9-dihydro-8H-purin-8-imine (A-45b)

(2-Chlorophenyl)boronic acid (147 mg, 0.94 mmol) and cesium carbonate (764 mg, 2.3 mmol) were added to a stirred solution of compound A-45a (500 mg, 0.5 mmol) in 1,4-dioxane (20 mL) in a sealed tube. The reaction mixture was degassed by bubbling with argon for 20 min, then XPhos-Pd-G2 (37 mg, 4.69 mmol) was added and the mixture was degassed for 10 minutes, then stirred at 110° C. for 16 h in a sealed tube. The reaction mixture was filtered through the celite bed, washed with EtOAc (100 mL) and the filtrate was washed with water and brine (2×100 mL). The combined organic layer was dried (Na₂SO₄), filtered and concentrated under reduced pressure. The afforded crude compound was purified by prep HPLC on X-Bridge C₁₈ (30×250) mm 10μ column using a gradient of 10 mM NH₄HCO₃ in H₂O: MeCN as mobile phase. The obtained residue was further purified by prep HPLC on Kromosil C18 (25×150) mm 10μ column using a gradient of 10 mM NH₄HCO₃ in H₂O: MeCN as mobile phase and lyophilised, which gave the title compound (20 mg, 8%) as a solid. MS (ES+) m/z 502.44 [M+H]⁺.

¹H NMR (500 MHz, DMSO): δ 7.62 (m, J=3.3 Hz, 3H), 7.46 (m, J=3.5 Hz, 5H), 7.35 (s, 1H), 6.76 (d, J=35.6 Hz, 1H), 5.15 (d, J=51.3 Hz, 2H), 3.68 (s, 3H), 3.60 (d, J=13.4 Hz, 3H), 2.18 (s, 3H).

### Example A-46

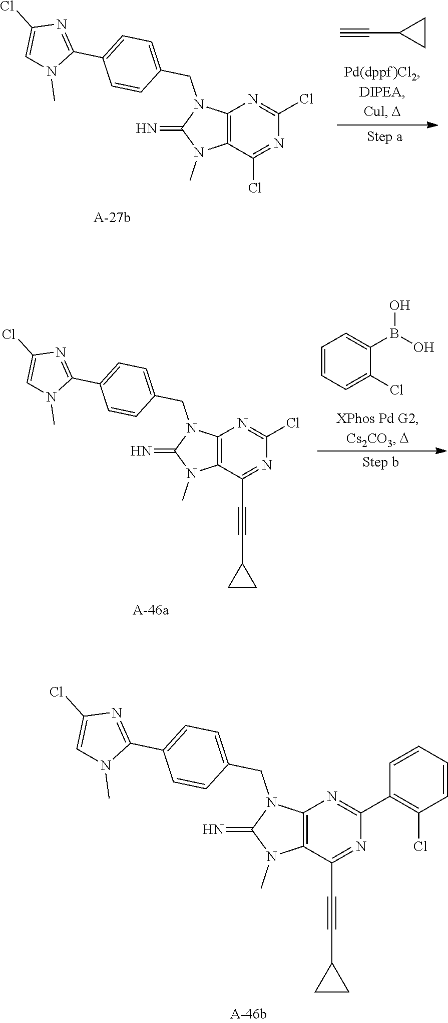

### Step a) 2-chloro-9-(4-(4-chloro-1-methyl-1H-imidazol-2-yl)benzyl)-6-(cyclopropylethynyl)-7-methyl-7,9-dihydro-8H-purin-8-imine (A-46a)

DIPEA (1.3 mL, 7.4 mmol), copper iodide (89 mg, 0.5 mmol) and Pd(dppf)Cl₂·DCM (155 mg, 0.2 mmol) were added at rt to a stirred solution of compound A-27b (1 g, 2.1 mmol) in DMF (12 mL). The reaction mixture was degassed by bubbling with argon for 15 minutes, then ethynylcyclopropane (210 mg, 3.2 mmol) was added and the reaction mixture was degassed by bubbling with argon for 5 minutes. The resulting reaction mixture was stirred at 110° C. for 16 h. The reaction mixture was ice water and extracted with DCM (3×30 mL). The combined organic layer was washed with brine (15 mL), dried (Na₂SO₄), filtered and concentrated under reduced pressure, which gave the title compound (1.3 g, 51%). MS (ES+) m/z 452.41 [M+H]⁺.

### Step b) 9-(4-(4-chloro-1-methyl-1H-imidazol-2-yl)benzyl)-2-(2-chlorophenyl)-6-(cyclopropylethynyl)-7-methyl-7,9-dihydro-8H-purin-8-imine (A-46b)

(2-Chlorophenyl)boronic acid (350 mg, 2.2 mmol) and cesium carbonate (1.8 g, 5.5 mmol) were added to a stirred solution of compound A-46a (1.3 g, 1.1 mmol) in 1,4-dioxane (20 mL) in a sealed tube. The reaction mixture was degassed by bubbling with argon for 15 min, then XPhos-Pd-G2 (90 mg, 0.11 mmol) was added and the mixture was degassed for 5 minutes, then stirred at 110° C. for 16 h. The reaction mixture was diluted with ice water and extracted with DCM (3×30 mL). The combined organic layer was dried (Na₂SO₄), filtered and concentrated under reduced pressure. The afforded crude compound was purified by column chromatography on silica gel, eluted with 3% MeOH/DCM. The obtained residue was further purified by prep HPLC on Kromosil C18 (25×150) mm 10μ column using a gradient of 10 mM NH₄HCO₃ in H₂O: MeCN as mobile phase, which gave the title compound (20 mg, 3%) as a solid. MS (ES+) m/z 528.53 [M+H]⁺.

¹H NMR (500 MHz, DMSO): δ 7.61 (m, J=1.8 Hz, 1H), 7.51 (m, J=2.3 Hz, 1H), 7.47 (d, J=8.2 Hz, 1H), 7.42 (m, J=2.7 Hz, 1H), 7.35 (s, 1H), 6.81 (s, 1H), 5.14 (s, 1H), 3.68 (s, 1H), 3.58 (s, 1H), 1.70 (m, J=4.1 Hz, 1H), 0.99 (m, J=3.2 Hz, 1H), 0.86 (m, J=2.6 Hz, 1H).

### Example B-1

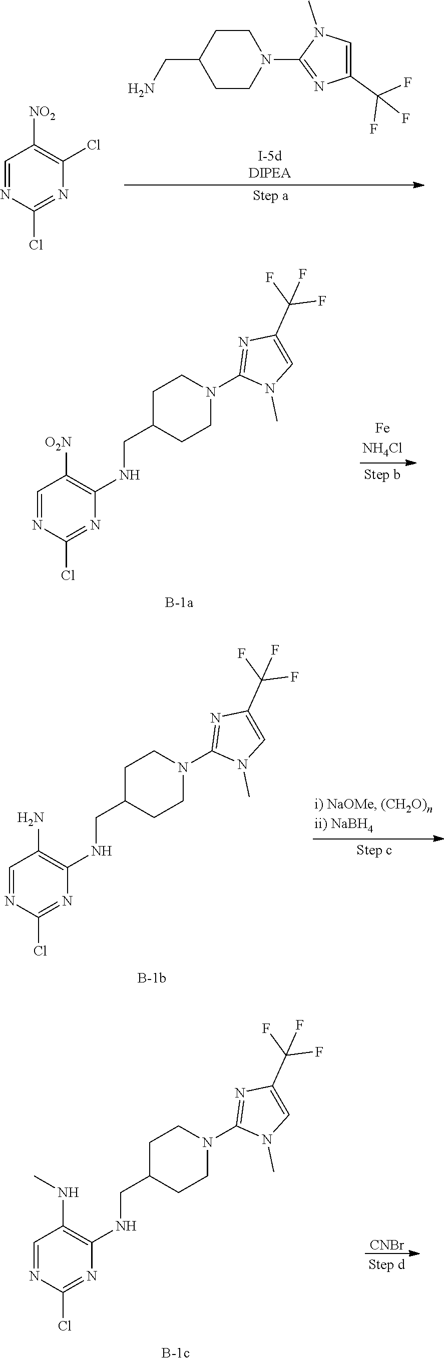 [SMILES: O=[N+]([O-])c1cnc(Cl)nc1Cl ; Cn1cc(C(F)(F)F)nc1N1CCC(CN)CC1 ; Cn1cc(C(F)(F)F)nc1N1CCC(CNc2nc(Cl)ncc2[N+](=O)[O-])CC1 ; Cn1cc(C(F)(F)F)nc1N1CCC(CNc2nc(Cl)ncc2N)CC1 ; CNc1cnc(Cl)nc1NCC1CCN(c2nc(C(F)(F)F)cn2C)CC1 ; "CNBr"]

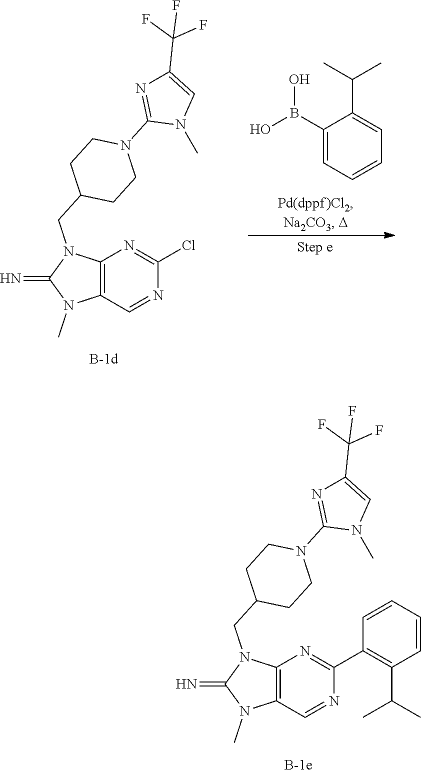 [SMILES: Cn1cc(C(F)(F)F)nc1N1CCC(Cn2c(=N)n(C)c3cnc(Cl)nc32)CC1 ; CC(C)c1ccccc1B(O)O ; CC(C)c1ccccc1-c1ncc2c(n1)n(CC1CCN(c3nc(C(F)(F)F)cn3C)CC1)c(=N)n2C]

### Step a) 2-chloro-N-((1-(1-methyl-4-(trifluoromethyl)-H-imidazol-2-yl)piperidin-4-yl)methyl)-5-nitropyrimidin-4-amine (B-1a)

DIPEA (2.12 mL, 12.2 mmol) was added to a stirred solution of 2,4-dichloro-5-nitropyrimidine (1.42 g, 7.32 mmol) and compound I-5d (1.6 g, 6.1 mmol) in DMF (5 mL) at 0° C. The resulting mixture was stirred for 16 h. at rt. Water (40 mL) was added and the mixture was extracted with EtOAc (2×50 mL). The organic layer was washed with water, brine, dried (Na₂SO₄), filtered and concentrated. The crude compound was purified by column chromatography on silica gel and eluted with 25% EtOAc/pet ether, which gave the title compound (1 g) as a solid. LCMS (ES+) 420.24 [M+H]⁺. The compound was taken to next step without further purification.

### Step b) 2-chloro-N4-((1-(1-methyl-4-(trifluoromethyl)-1H-imidazol-2-yl)piperidin-4-yl)methyl)pyrimidine-4,5-diamine (B-1b)

Fe (472 mg, 8.50 mmol) and NH₄C₁ (180 mg, 3.40 mmol) were added at rt to a stirred solution of compound B-1a (1 g, 1.7 mmol) in ethanol (15 mL), THE (15 mL) and water (6 mL). The resulting reaction mixture was heated at 80° C. for 2 h, then filtered through the celite bed. The filtrate was diluted with water (20 mL) extracted with EtOAc (2×50 mL). The combined organic layers were washed with brine, dried (Na₂SO₄), filtered and concentrated. The crude compound was triturated with diethyl ether (2×5 mL) and n-pentane (2×5 mL), which gave the title compound (800 mg) as a solid. MS (ES+) 390.34 [M+H]⁺.

### Step c) 2-chloro-N5-methyl-N4-((1-(1-methyl-4-(trifluoromethyl)-1H-imidazol-2-yl)piperidin-4-yl)methyl)pyrimidine-4,5-diamine (B-1c)

NaOMe (2.5M in MeOH) (1.2 mL, 5.13 mmol) was added at 0° C. to a stirred solution of compound B-1b (800 mg, 2.1 mmol) and paraformaldehyde (93 mg, 3.1 mmol) in MeOH (5 mL). The mixture was heated for 2 h at 60° C., then NaBH₄ (194 mg, 5.13 mmol) was added at 0° C. The mixture was stirred at rt for 2 h, then concentrated. Water (5 mL) was added to the residue and extracted with EtOAc (2×25 mL). The combined organic layers were washed with brine, dried (Na₂SO₄), filtered and concentrated. The crude compound was triturated with diethyl ether (2×5 mL) and n-pentane (2×5 mL), which gave the title compound (500 mg, 57%) as a solid. MS (ES+) 404.32 [M+H]⁺.

### Step d) 2-chloro-7-methyl-9-((1-(1-methyl-4-(trifluoromethyl)-1H-imidazol-2-yl)piperidin-4-yl)methyl)-7H-purin-8(9H)-imine (B-1d)

Cyanogen bromide (420 mg, 4.00 mmol) was added at 0° C. to a stirred solution of compound B-1c (450 mg, 1.00 mmol) in EtOH (15 mL). The resulting mixture was stirred at 80° C. for 4 h, then concentrated. The residue was dissolved in water (20 mL) and extracted with EtOAc (2×25 mL). The combined organic layers were washed with brine, dried (Na₂SO₄), filtered and concentrated under reduced pressure. The crude compound was triturated with diethyl ether (2×5 mL) and n-pentane (2×5 mL), which gave the crude title compound (500 mg) as a liquid. MS (ES+) 429.26 [M+H]⁺. The compound was taken to next step without further purification.

### Step e) 2-(2-isopropylphenyl)-7-methyl-9-((1-(1-methyl-4-(trifluoromethyl)-1H-imidazol-2-yl)piperidin-4-yl)methyl)-7H-purin-8(9H)-imine (B-1e)

Sodium carbonate (130 mg, 1.22 mmol) and (2-isopropylphenyl) boronic acid (96 mg, 0.60 mmol) were added to a stirred solution of compound B-1d (500 mg, 0.50 mmol) in 1,4-dioxane (6 mL) and water (2 mL) in a sealed tube. The reaction mixture was degassed by bubbling with argon for 10 minutes then Pd(dppf)Cl₂·DCM, (36 mg, 0.05 mmol) was added and the reaction mixture was stirred at 120° C. for 16 h in a sealed tube. The reaction mixture was concentrated, diluted with water (10 mL), extracted with EtOAc (3×25 mL) and the combined organic layers were dried (Na₂SO₄) and concentrated. The crude product was purified by prep HPLC on a Kromosil C18 (25×150) mm 10μ column using a gradient of 10 mM NH₄HCO₃ in H₂O: MeCN as mobile phase. The title compound was further purified by prep HPLC on an X-Select C18 (19×150) mm 5μ column using 0.1% formic acid in H₂O: MeCN as mobile phase. Water (20 mL) was added to the obtained compound, adjusted pH to 9 by adding saturated NaHCO₃ solution and extracted with DCM (2×50 mL), dried (Na₂SO₄) and concentrated, which gave the title compound (52 mg, 20%) as a solid. LCMS (ES+) m/z 513.43 [M+H]⁺.

¹H NMR (500 MHz, DMSO) δ 8.21 (s, 1H), 7.50 (m, J=2.9 Hz, 2H), 7.43 (d, J=7.1 Hz, 1H), 7.38 (m, J=3.3 Hz, 1H), 7.24 (m, J=3.2 Hz, 1H), 6.54 (s, 1H), 3.83 (d, J=7.1 Hz, 2H), 3.53 (t, J=6.9 Hz, 1H), 3.46 (s, 3H), 3.37 (s, 3H), 3.26 (t, J=10.2 Hz, 2H), 2.66 (t, J=11.4 Hz, 2H), 2.07 (s, 1H), 1.66 (d, J=11.1 Hz, 2H), 1.44 (m, J=6.1 Hz, 2H), 1.18 (d, J=6.9 Hz, 6H).

### Example B-2

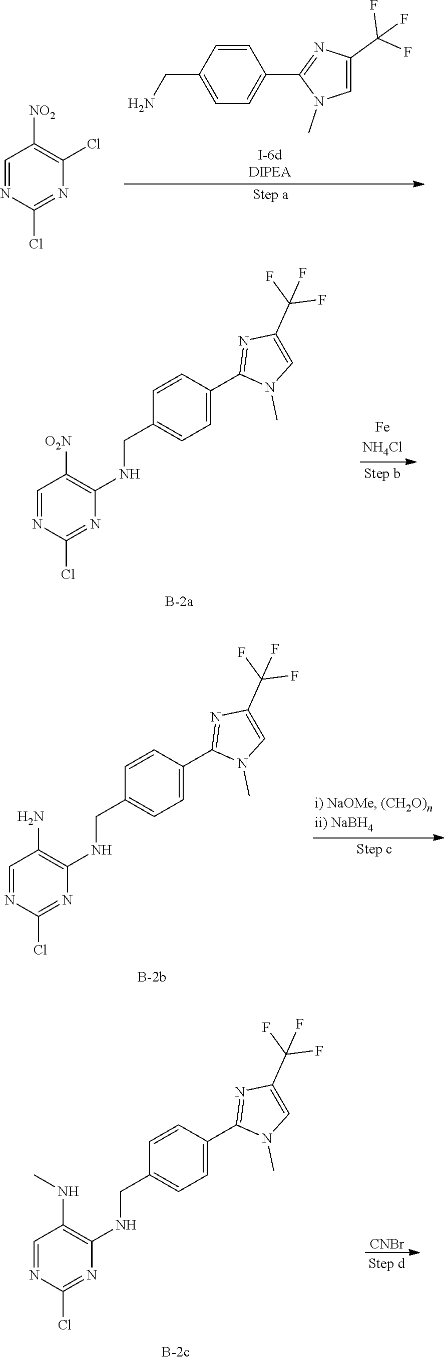 [SMILES: O=[N+]([O-])c1cnc(Cl)nc1Cl ; Cn1cc(C(F)(F)F)nc1-c1ccc(CN)cc1 ; Cn1cc(C(F)(F)F)nc1-c1ccc(CNc2nc(Cl)ncc2[N+](=O)[O-])cc1 ; Cn1cc(C(F)(F)F)nc1-c1ccc(CNc2nc(Cl)ncc2N)cc1 ; CNc1cnc(Cl)nc1NCc1ccc(-c2nc(C(F)(F)F)cn2C)cc1 ; "CNBr"]

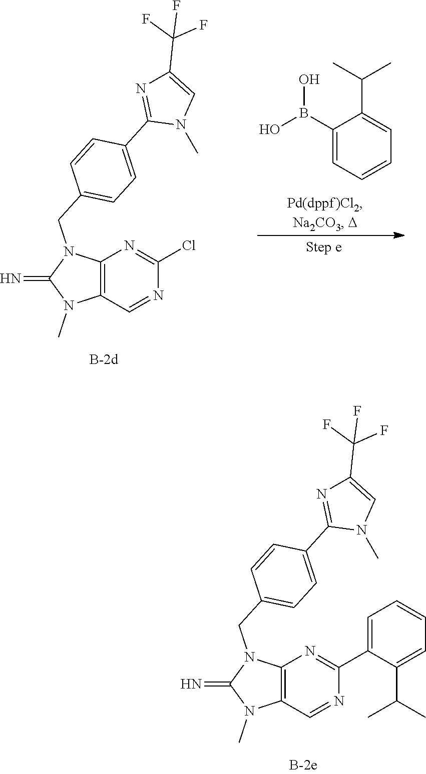 [SMILES: Cn1cc(C(F)(F)F)nc1-c1ccc(Cn2c(=N)n(C)c3cnc(Cl)nc32)cc1 ; CC(C)c1ccccc1B(O)O ; CC(C)c1ccccc1-c1ncc2c(n1)n(Cc1ccc(-c3nc(C(F)(F)F)cn3C)cc1)c(=N)n2C]

### Step a) 2-chloro-N-(4-(1-methyl-4-(trifluoromethyl)-1H-imidazol-2-yl)benzyl)-5-nitropyrimidin-4-amine (B-2a)

DIPEA (13.7 mL, 78.4 mmol) was added to a stirred solution of 2,4-dichloro-5-nitropyrimidine (9.1 g, 47.0 mmol) and compound I-6b (10 g, 39.2 mmol) in DMF (45 mL) at 0° C. The resulting mixture was stirred for 16 h. at rt. Water (80 mL) was added and the mixture was extracted with EtOAc (2×100 mL). The organic layer was washed with brine, dried (Na₂SO₄), filtered and concentrated. The crude compound was purified by column chromatography on silica gel and eluted with 25% EtOAc/pet ether, which gave the title compound (4.1 g) as a liquid. LCMS (ES+) 413.12 [M+H]⁺. The compound was taken to next step without further purification.

### Step b) 2-chloro-N4-(4-(1-methyl-4-(trifluoromethyl)-1H-imidazol-2-yl)benzyl)pyrimidine-4,5-diamine (B-2b)

Fe (2.6 g, 46.03 mmol) and NH₄C₁ (985 mg, 18.41 mmol) were added at rt to a stirred solution of compound B-2a (4 g, 9.20 mmol) in ethanol (20 mL), THE (20 mL) and water (8 mL). The resulting reaction mixture was heated at 80° C. for 2 h, then filtered through a celite bed. The filtrate was concentrated, the crude compound was purified by column chromatography on silica gel and eluted with 75% EtOAc/pet ether, which gave the title compound (2.5 g, 64%) as a solid. LCMS (ES+) 383.19 [M+H]⁺.

### Step c) 2-chloro-N5-methyl-N4-(4-(1-methyl-4-(trifluoromethyl)-1H-imidazol-2-yl)benzyl)pyrimidine-4,5-diamine (B-2c)

A solution of NaOMe (25%, 2.80 g, 12.80 mmol) in MeOH was added at 0° C. to a stirred solution of compound B-2b (1 g, 2.6 mmol) and paraformaldehyde (115 mg, 3.84 mmol) in MeOH (20 mL). The mixture was heated for 16 h at 60° C., then NaBH₄ (242 mg, 6.40 mmol) was added at 0° C. The mixture was stirred at rt for 16 h, then concentrated. Water (5 mL) was added to the residue and extracted with EtOAc (2×75 mL). The combined organic layers were washed with brine, dried (Na₂SO₄), filtered and concentrated. The crude compound was purified by column chromatography on silica gel and eluted with 75% EtOAc/pet ether, which gave the title compound (200 mg) as a solid. MS (ES+) 397.21 [M+H]⁺. The compound was taken to next step without further purification.

### Step d) 2-chloro-7-methyl-9-(4-(1-methyl-4-(trifluoromethyl)-1H-imidazol-2-yl)benzyl)-7H-purin-8(9H)-imine (B-2d)

Cyanogen bromide (111 mg, 1.1 mmol) was added at 0° C. to a stirred solution of compound B-2c (180 mg, 0.44 mmol) in EtOH (5 mL). The resulting mixture was stirred at 80° C. for 16 h. Another lot of cyanogen bromide (56 mg, 0.53 mmol) was added and heated at 80° C. for 8 h, then concentrated. The residue was dissolved in water (20 mL) and extracted with EtOAc (2×25 mL). The combined organic layers were washed with brine, dried (Na₂SO₄), filtered and concentrated under reduced pressure. The crude compound was triturated with diethyl ether (2×5 mL) and n-pentane (2×5 mL), which gave the crude title compound (100 mg) as a solid. MS (ES+) 422.24 [M+H]⁺. The compound was taken to next step without further purification.

### Step e) 2-(2-isopropylphenyl)-7-methyl-9-(4-(1-methyl-4-(trifluoromethyl)-1H-imidazol-2-yl)benzyl)-7H-purin-8(9H)-imine (B-2e)

Sodium carbonate (25 mg, 0.23 mmol) and (2-isopropylphenyl) boronic acid (23 mg, 0.14 mmol) were added to a stirred solution of compound B-2d (100 mg, 0.1 mmol) in 1,4-dioxane (3 mL) and water (1 mL) in a sealed tube. The reaction mixture was degassed by bubbling with argon for 10 minutes then Pd(dppf)Cl₂·DCM, (7 mg, 0.01 mmol) was added and the reaction mixture was stirred at 100° C. for 16 h in a sealed tube. The reaction mixture was concentrated, diluted with water (10 mL), extracted with EtOAc (3×25 mL) and the combined organic layers were dried (Na₂SO₄). The crude compound was purified by column chromatography on silica gel and eluted with 5% MeOH/DCM. The residue was purified by prep HPLC on an X-Select C18 (19×150) mm 5μ column using 0.1% formic acid in H₂O: MeCN as mobile phase, which gave the title compound (12 mg, 24%) as a solid. LCMS (ES+) m/z 506.28 [M+H]⁺.

¹H NMR (500 MHz, DMSO): δ 8.23 (s, 1H), 7.91 (d, J=0.9 Hz, 1H), 7.67 (d, J=8.3 Hz, 2H), 7.48 (m, J=2.6 Hz, 3H), 7.38 (m, J=3.7 Hz, 2H), 7.23 (m, J=3.2 Hz, 1H), 5.17 (s, 2H), 3.75 (s, 3H), 3.39 (s, 3H), 1.09 (d, J=6.9 Hz, 6H).

### Example B-3

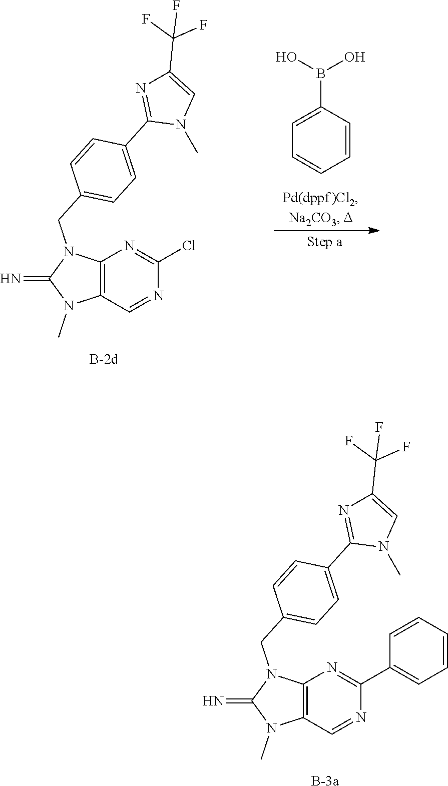

### Step a) 7-methyl-9-(4-(1-methyl-4-(trifluoromethyl)-1H-imidazol-2-yl)benzyl)-2-phenyl-7,9-dihydro-8H-purin-8-imine (B-3a)

Sodium carbonate (111 mg, 1.0 mmol) and phenylboronic acid (213 mg, 1.74 mmol) were added to a stirred solution of compound B-2d (160 mg, 0.4 mmol) in 1,4-dioxane (10 mL) and water (3 mL) in a sealed tube. The reaction mixture was degassed by bubbling with argon for 10 minutes then Pd(dppf)Cl₂·DCM, (128 mg, 0.2 mmol) was added and the reaction mixture was stirred at 100° C. for 16 h in a sealed tube. The reaction mixture was concentrated, diluted with water (10 mL), extracted with EtOAc (2×25 mL). The combined organic layers were washed with brine (10 mL), dried (Na₂SO₄), filtered and concentrated under reduced pressure. The crude compound was purified by prep HPLC on an X-Select C18 (19×150) mm 5μ column using 0.1% formic acid in H₂O: MeCN as mobile phase which gave the title compound (40 mg, 24%) as a solid. LCMS (ES+) m/z 464.44 [M+H]⁺.

¹H NMR (500 MHz, DMSO): δ 8.32 (m, J=2.0 Hz, 2H), 8.21 (s, 1H), 7.91 (d, J=1.1 Hz, 1H), 7.69 (d, J=8.3 Hz, 2H), 7.58 (d, J=7.7 Hz, 2H), 7.45 (m, J=3.4 Hz, 3H), 6.52 (s, 1H), 5.20 (s, 2H), 3.74 (s, 3H), 3.38 (s, 3H).

### Example B-4

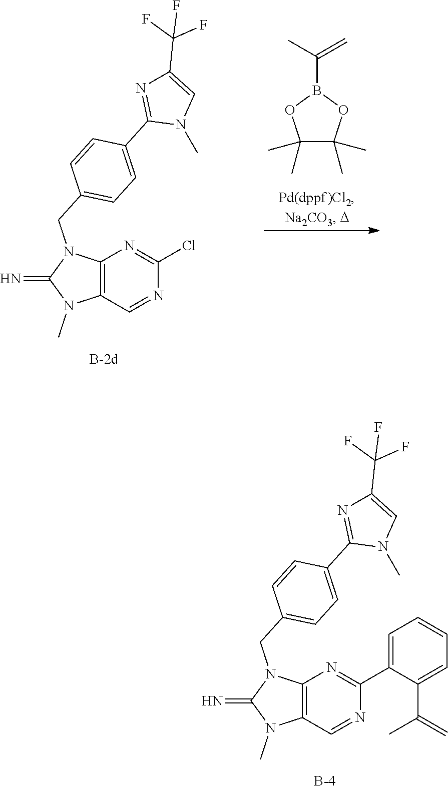

### 7-methyl-9-(4-(1-methyl-4-(trifluoromethyl)-1H-imidazol-2-yl)benzyl)-2-(2-(prop-1-en-2-yl)phenyl)-7,9-dihydro-8H-purin-8-imine (B-4)

Cesium carbonate (243 mg, 0.74 mmol) and 4,4,5,5-tetramethyl-2-(prop-1-en-2-yl)-1,3,2-dioxaborolane (65 mg, 0.4 mmol) were added to a stirred solution of compound B-3a (390 mg, 0.3 mmol) in toluene (10 mL) in a sealed tube. The reaction mixture was degassed by bubbling with argon for 15 minutes then Pd(PPh₃)₄ (34 mg, 0.03 mmol) was added and the reaction mixture was stirred at 100° C. for 12 h in a sealed tube. The reaction mixture was concentrated and diluted with water, extracted with EtOAc. The combined organic layers were washed with brine, dried (Na₂SO₄), filtered and concentrated under reduced pressure. The afforded crude compound was purified by column chromatography on silica gel, eluted with 7% MeOH/DCM. The residue was purified by prep HPLC on an X-Select C18 (19×150) mm 5μ column using 10 mM NH₄HCO₃ in H₂O: MeCN as mobile phase. The residue was further purified by Prep SFC, which gave the title compound (30 mg, 20%) as a solid. LCMS (ES+) m/z 504.50 [M+H]⁺.

¹H NMR (500 MHz, DMSO): δ 8.21 (s, 1H), 7.92 (d, J=0.9 Hz, 1H), 7.68 (q, J=5.5 Hz, 3H), 7.49 (d, J=8.3 Hz, 2H), 7.36 (m, J=3.2 Hz, 2H), 7.26 (m, J=2.2 Hz, 1H), 6.57 (s, 1H), 5.13 (s, 2H), 4.85 (s, 1H), 4.61 (d, J=1.0 Hz, 1H), 3.75 (s, 3H), 3.37 (s, 3H), 1.78 (s, 3H).

Preparative SFC Conditions
Column/dimensions: Chiralcel AD-H (250×4.6 mm), 5p
CO₂: 80.0%
Co solvent: 20.0% (30 mM methanolic ammonia in methanol)
Total flow: 70.0 g/min
Back pressure: 90.0 bar
UV: 214 nm
Stack time: 10.5 min
Load/Inj.: 9.5 mg

### Example B-5

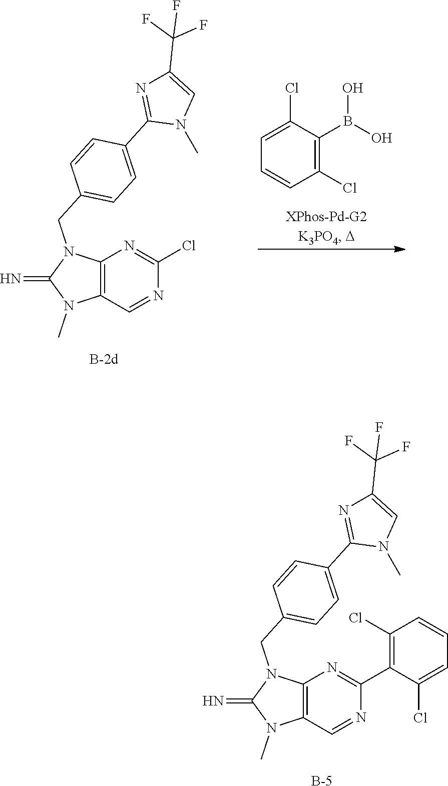

### 2-(2,6-dichlorophenyl)-7-methyl-9-(4-(1-methyl-4-(trifluoromethyl)-1H-imidazol-2-yl)benzyl)-7,9-dihydro-8H-purin-8-imine (B-5)

(2,6-dichlorophenyl)boronic acid (132 mg, 0.7 mmol) and K3PO4 (80.3 mg, 0.4 mmol) was added to a stirred solution of compound B-3a (350 mg, 0.63 mmol) in THE (8 mL) and water (2 mL). The reaction mixture was degassed for 10 min with argon, Xphos-Pg-G2 (20 mg, 0.03 mmol) was added and the resulting reaction mixture was stirred at 60° C. for 2 h in microwave. The reaction mixture was diluted with ice water and extracted with EtOAc. The combined organic layers were washed with water, brine, dried (Na₂SO₄), filtered and concentrated under reduced pressure. The crude compound was purified by column chromatography on silica gel, eluted with 8% MeOH in DCM. The residue was further purified by prep HPLC on Kromosil C18 (25×150) mm 10μ column using a gradient of 10 mM NH₄HCO₃ in H₂O: MeCN as mobile phase. The impure product was further purified by prep HPLC on Kromosil C18 (25×150) mm 10μ column using a gradient of 0.1% formic acid in H₂O: MeCN as mobile phase, which gave the title compound (22 mg, 6%) as a solid. LCMS (ES+) 532.45 [M+H]⁺.

¹H NMR (500 MHz, DMSO): δ 8.36 (s, 1H), 7.92 (s, 1H), 7.65 (d, J=8.2 Hz, 2H), 7.57 (d, J=7.8 Hz, 2H), 7.48 (q, J=6.0 Hz, 3H), 5.20 (s, 2H), 3.74 (s, 3H), 3.43 (s, 3H).

### Example B-7

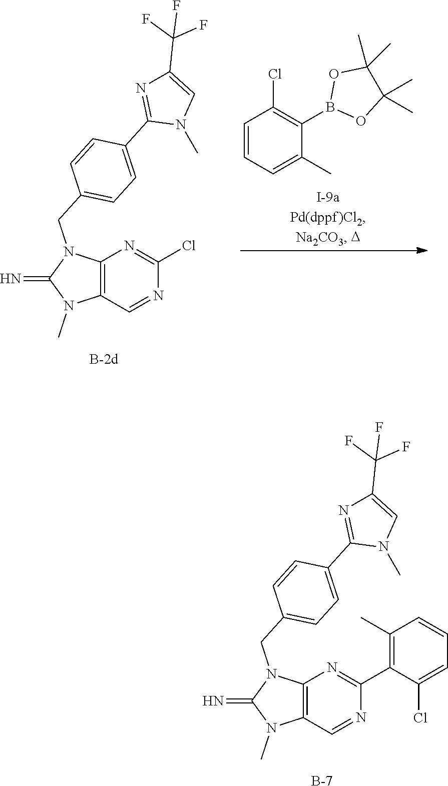

### 2-(2-chloro-6-methylphenyl)-7-methyl-9-(4-(1-methyl-4-(trifluoromethyl)-1H-imidazol-2-yl)benzyl)-7,9-dihydro-8H-purin-8-imine (B-7)

Sodium carbonate (238 mg, 2.3 mmol) and compound I-9a (310 mg, 1.13 mmol) were added to a stirred solution of compound B-3a (250 mg, 0.45 mmol) in 1,4-dioxane (20 mL) and water (10 mL) in a sealed tube. The reaction mixture was degassed by bubbling with argon for 15 minutes then Pd(dppf)Cl₂·DCM, (37 mg, 0.05 mmol) was added and the reaction mixture was stirred at 110° C. for 16 h in a sealed tube. The reaction mixture was diluted with water (10 mL), extracted with EtOAc (3×25 mL). The combined organic layers were washed with brine (20 mL), dried (Na₂SO₄), filtered and concentrated under reduced pressure. The afforded crude compound was purified by by column chromatography on silica gel, eluted with 4% MeOH/DCM. The residue was purified by prep HPLC on Kromosil C18 (25×150) mm 10μ column using a gradient of 10 mM NH₄HCO₃ in H₂O: MeCN as mobile phase, which gave the title compound (12 mg, 5%) as a solid. LCMS (ES+) m/z 512.46 [M+H]⁺.

¹H NMR (500 MHz, DMSO): δ 10.28 (s, 1H), 7.64 (d, J=30.2 Hz, 1H), 7.52 (d, J=8.5 Hz, 2H), 6.92 (d, J=8.4 Hz, 2H), 5.72 (s, 1H), 3.00 (m, J=10.8 Hz, 2H), 2.07 (m, J=6.3 Hz, 2H), 1.38 (m, J=15.7 Hz, 7H).

### Example B-8

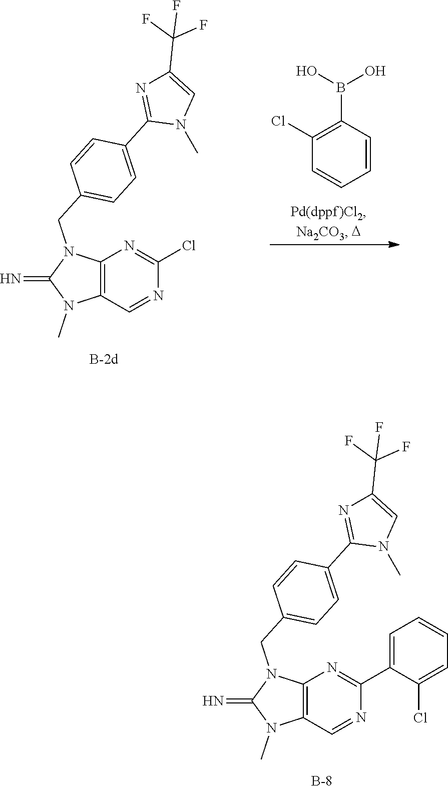

### 2-(2-chlorophenyl)-7-methyl-9-(4-(1-methyl-4-(trifluoromethyl)-1H-imidazol-2-yl)benzyl)-7,9-dihydro-8H-purin-8-imine (B-8)

Sodium carbonate (145 mg, 1.4 mmol) and (2-chlorophenyl)boronic acid (112 mg, 0.7 mmol) were added to a stirred solution of compound B-3a (300 mg, 0.6 mmol) in 1,4-dioxane (9 mL) and water (3 mL). The reaction mixture was degassed by bubbling with argon for 15 minutes then Pd(dppf)Cl₂·DCM, (41 mg, 0.05 mmol) was added and the reaction mixture was stirred at 100° C. for 1 h in microwave. The reaction mixture was concentrated and diluted with water and extracted with EtOAc. The combined organic layers were washed with brine, dried (Na₂SO₄), filtered and concentrated under reduced pressure. The afforded crude compound was purified by column chromatography on silica gel, eluted with 7% MeOH/DCM. The residue was purified by prep HPLC on an YMC Trait C18 (25×150) mm 10μ column using a gradient of 10 mM NH₄HCO₃ in H₂O: MeCN as mobile phase, which gave the title compound (41 mg, 15%) as a solid. LCMS (ES+) m/z 498.48 [M+H]⁺.

¹H NMR (500 MHz, DMSO): δ 8.23 (s, 1H), 7.91 (d, J=1.1 Hz, 1H), 7.67 (m, J=2.3 Hz, 3H), 7.53 (m, J=2.0 Hz, 3H), 7.43 (m, J=2.1 Hz, 1H), 6.58 (d, J=36.0 Hz, 1H), 5.17 (d, J=44.0 Hz, 2H), 3.75 (s, 3H), 3.38 (d, J=9.1 Hz, 3H).

### Example B-9

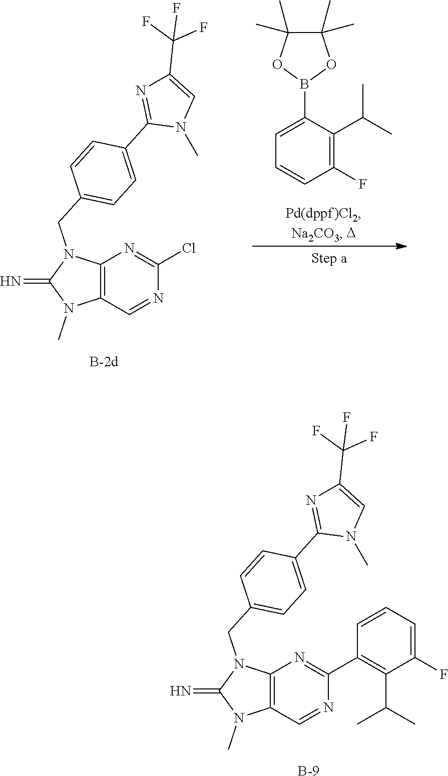

### 2-(3-fluoro-2-isopropylphenyl)-7-methyl-9-(4-(1-methyl-4-(trifluoromethyl)-1H-imidazol-2-yl)benzyl)-7,9-dihydro-8H-purin-8-imine (B-9)

A stirred solution of compound B-3a (300 mg, 0.5 mmol) and 2-(3-fluoro-2-isopropylphenyl)-4,4,5,5-tetramethyl-1,3,2-dioxaborolane (285 mg, 0.65 mmol) in 1,4-dioxane (10 mL) and water (3 mL) was degassed by bubbling with argon for 10 minutes, then Sodium carbonate (132 mg, 1.2 mmol) and Pd(dppf)Cl₂·DCM, (41 mg, 0.05 mmol) were added and the reaction mixture was stirred at 80° C. for 16 h. The reaction mixture was diluted with water, filtered through the celite bed. The filtrate was extracted with EtOAc. The combined organic layer was washed with brine, dried (Na₂SO₄), filtered and concentrated under reduced pressure. The afforded crude compound was purified by column chromatography on silica gel, eluted with 5% MeOH/DCM. The residue was purified by prep HPLC on Kromosil C18 (25×150) mm 10p column using a gradient of 10 mM NH₄HCO₃ in H₂O: MeCN as mobile phase, which gave the title compound (119 mg, 45%) as a solid. LCMS (ES+) m/z 524.53 [M+H]⁺.

¹H NMR (500 MHz, DMSO): δ 8.24 (d, J=25.1 Hz, 1H), 7.92 (s, 1H), 7.67 (d, J=7.1 Hz, 2H), 7.47 (q, J=7.4 Hz, 2H), 7.27 (m, J=2.6 Hz, 2H), 7.16 (m, J=3.1 Hz, 1H), 6.56 (d, J=34.1 Hz, 1H), 5.17 (d, J=50.8 Hz, 2H), 3.75 (s, 3H), 3.38 (d, J=15.1 Hz, 3H), 3.29 (d, J=8.5 Hz, 1H), 1.19 (d, J=6.7 Hz, 6H).

### Example B-10

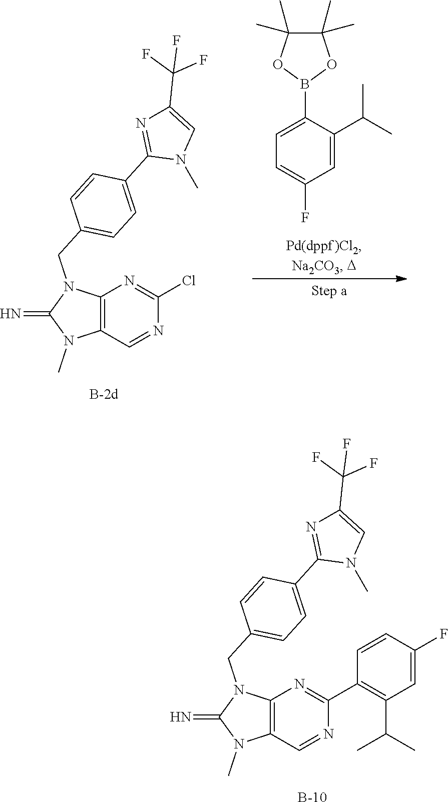

### Step a) 2-(4-fluoro-2-isopropylphenyl)-7-methyl-9-(4-(1-methyl-4-(trifluoromethyl)-1H-imidazol-2-yl)benzyl)-7,9-dihydro-8H-purin-8-imine (B-10)

2-(4-fluoro-2-isopropylphenyl)-4,4,5,5-tetramethyl-1,3,2-dioxaborolane (132 mg, 0.5 mmol) and sodium carbonate (93 mg, 0.9 mmol) were added to a stirred solution of compound B-3a (150 mg, 0.35 mmol) in 1,4-dioxane (12 mL) and water (3 mL) in a sealed tube. The reaction mixture was degassed by bubbling with argon for 15 minutes then Pd(dppf)Cl₂·DCM, (26 mg, 0.04 mmol) was added and the reaction mixture was degassed by bubbling with argon for 5 minutes and stirred at 100° C. for 16 h in a sealed tube. The reaction mixture was filtered through the celite bed. The filtrate was concentrated under reduced pressure. The afforded crude compound was purified by column chromatography on silica gel, eluted with 6% MeOH/DCM. The residue was purified by prep HPLC on Kromosil C18 (25×150) mm 10μ column using a gradient of 10 mM NH₄HCO₃ in H₂O: MeCN as mobile phase and lyophilised, which gave the title compound (50 mg, 27%) as a solid. LCMS (ES+) m/z 524.53 [M+H]⁺.

¹H NMR (500 MHz, DMSO): δ 8.23 (s, 1H), 7.92 (d, J=1.1 Hz, 1H), 7.68 (d, J=8.4 Hz, 2H), 7.56 (q, J=5.0 Hz, 1H), 7.47 (d, J=8.3 Hz, 2H), 7.20 (q, J=4.6 Hz, 1H), 7.06 (m, J=3.9 Hz, 1H), 6.1 (s, 1H), 5.17 (s, 2H), 3.75 (s, 3H), 3.51 (m, J=3.4 Hz, 1H), 3.39 (s, 3H), 1.09 (d, J=6.9 Hz, 6H).

### Example B-11

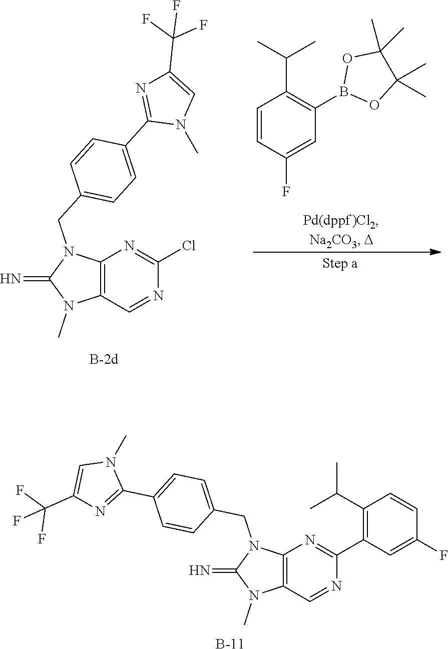

### 2-(5-fluoro-2-isopropylphenyl)-7-methyl-9-(4-(1-methyl-4-(trifluoromethyl)-1H-imidazol-2-yl)benzyl)-7,9-dihydro-8H-purin-8-imine (B-11)

A stirred solution of compound B-3a (450 mg, 0.7 mmol) and 2-(5-fluoro-2-isopropylphenyl)-4,4,5,5-tetramethyl-1,3,2-dioxaborolane (491 mg, 0.93 mmol) in 1,4-dioxane (10 mL) and water (3 mL) was degassed by bubbling with argon for 10 minutes, then sodium carbonate (190 mg, 2.0 mmol) followed by Pd(dppf)Cl₂·DCM (58 mg, 0.07 mmol) were added and the reaction mixture was stirred at 80° C. for 16 h. The reaction mixture was diluted with water and filtered through the celite bed. The filtrate was extracted with EtOAc. The combined organic layer was washed with brine, dried (Na₂SO₄), filtered and concentrated under reduced pressure. The afforded crude compound was purified by column chromatography on silica gel, eluted with 5% MeOH/DCM. The residue was further purified by prep HPLC on Kromosil C18 (25×150) mm 10μ column using a gradient of 10 mM NH₄HCO₃ in H₂O: MeCN as mobile phase, which gave the title compound (91 mg, 24%) as a solid. LCMS (ES+) m/z 524.57 [M+H]⁺.

¹H NMR (500 MHz, DMSO): δ 8.25 (d, J=24.7 Hz, 1H), 7.91 (d, J=0.9 Hz, 1H), 7.67 (d, J=7.1 Hz, 2H), 7.45 (m, J=6.5 Hz, 3H), 7.28 (q, J=4.3 Hz, 1H), 7.20 (m, J=4.0 Hz, 1H), 6.57 (d, J=37.5 Hz, 1H), 5.18 (d, J=50.6 Hz, 2H), 3.75 (s, 3H), 3.48 (d, J=6.1 Hz, 1H), 3.39 (d, J=14.4 Hz, 3H), 1.08 (d, J=6.8 Hz, 6H).

### Example C-1

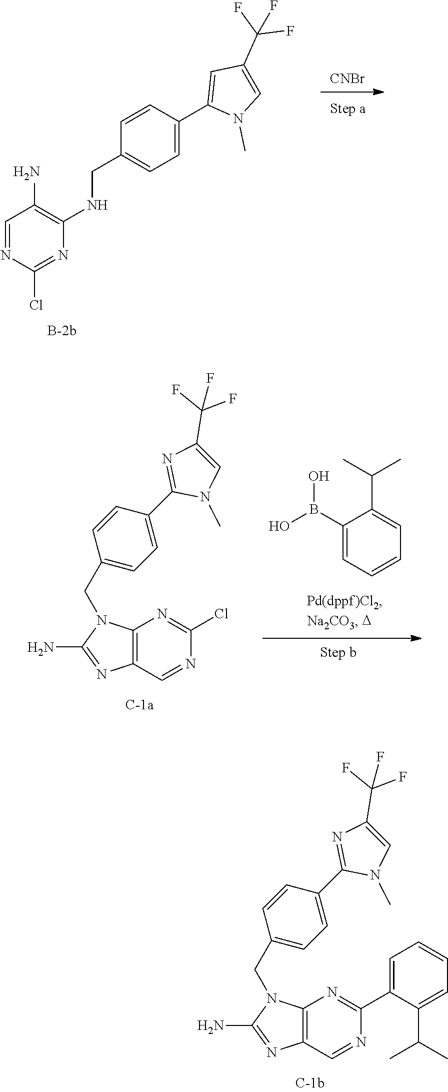

### Step a) 2-chloro-9-(4-(1-methyl-4-(trifluoromethyl)-1H-imidazol-2-yl)benzyl)-9H-purin-8-amine (C-1a)

Cyanogen bromide (390 mg, 3.70 mmol) was added at 0° C. to a stirred solution of compound B-2b (400 mg, 0.92 mmol) in EtOH (5 mL). The resulting mixture was stirred at 80° C. for 4 h, then concentrated. The residue was dissolved in water (20 mL) and extracted with EtOAc (2×25 mL). The combined organic layers were washed with brine, dried (Na₂SO₄), filtered and concentrated under reduced pressure. The crude compound was purified by column chromatography on silica gel and eluted with 10% MeOH/DCM (1% Et₃N), which gave the title compound (150 mg) as a solid. MS (ES+) 408.19 [M+H]⁺. The compound was taken to next step without further purification.

### Step b) 2-(2-isopropylphenyl)-9-(4-(1-methyl-4-(trifluoromethyl)-1H-imidazol-2-yl)benzyl)-9H-purin-8-amine (C-1b)

Sodium carbonate (77 mg, 0.73 mmol) and (2-isopropylphenyl) boronic acid (72 mg, 0.44 mmol) were added to a stirred solution of compound C-1a (150 mg, 0.29 mmol) in 1,4-dioxane (9 mL) and water (3 mL) in a sealed tube. The reaction mixture was degassed by bubbling with argon for 10 minutes then Pd(dppf)Cl₂·DCM, (21 mg, 0.03 mmol) was added and the reaction mixture was stirred at 100° C. for 16 h in a sealed tube. The reaction mixture was concentrated, diluted with water (10 mL), extracted with EtOAc (3×25 mL) and the combined organic layers were dried (Na₂SO₄). The crude compound was purified by column chromatography on silica gel and eluted with 75% EtOAc/pet ether. The impure compound was purified by prep HPLC on a Kromosil C18 (25×150) mm 10μ column using a gradient of 10 mM NH₄HCO₃ in H₂O: MeCN as mobile phase, which gave the title compound (30 mg, 20%) as a solid. LCMS (ES+) m/z 492.27 [M+H]⁺.

¹H NMR (500 MHz, DMSO): δ 8.53 (s, 1H), 7.91 (s, 1H), 7.69 (d, J=8.3 Hz, 2H), 7.52 (d, J=7.2 Hz, 1H), 7.38 (q, J=7.3 Hz, 6H), 7.23 (t, J=6.9 Hz, 1H), 5.39 (s, 2H), 3.74 (s, 3H), 3.48 (m, J=6.8 Hz, 1H), 1.08 (d, J=6.9 Hz, 6H).

### Example D-1 & D-2

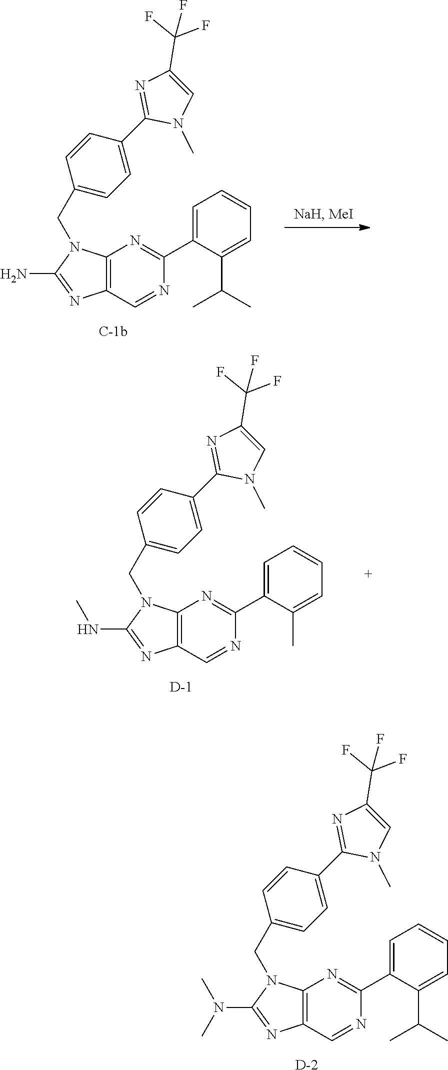

### 2-(2-isopropylphenyl)-N-methyl-9-(4-(1-methyl-4-(trifluoromethyl)-1H-imidazol-2-yl)benzyl)-9H-purin-8-amine (D-1)

### 2-(2-isopropylphenyl)-N,N-dimethyl-9-(4-(1-methyl-4-(trifluoromethyl)-1H-imidazol-2-yl)benzyl)-9H-purin-8-amine (D-2)

NaH (60%, 10 mg, 0.26 mmol) and CH₃I (0.02 mL, 0.26 mmol) were added at 0° C. to a solution of compound C-1b (150 mg, 0.26 mmol) in THE (10 ml). The mixture was stirred for 90 min at 0° C., then ice cold water (40 mL) was added and the mixture was extracted with EtOAc (2×75 mL). The combined organic layers were washed with brine, dried (Na₂SO₄), filtered and concentrated. The crude compound was purified by prep HPLC on an X-Select C18 (19×150) mm 5μ column using a gradient of 10 mM NH₄₀Ac in H₂O: MeCN as mobile phase, which gave the title compounds (8-1: 22 mg, 16%) & (8-2: 12 mg, 9%) as solids.

### D-1

MS (ES+) 506.44 [M+H]⁺.

¹H NMR (500 MHz, DMSO): δ 8.60 (s, 1H), 7.91 (d, J=0.9 Hz, 1H), 7.69 (d, J=8.3 Hz, 2H), 7.55 (m, J=5.4 Hz, 2H), 7.37 (m, J=4.1 Hz, 4H), 7.23 (m, J=3.2 Hz, 1H), 5.38 (s, 2H), 3.74 (s, 3H), 3.48 (m, J=6.8 Hz, 1H), 3.00 (d, J=4.5 Hz, 3H), 1.08 (d, J=6.9 Hz, 6H).

### D-2

MS (ES+) 520.41 [M+H]⁺.

¹H NMR (500 MHz, DMSO): δ 8.78 (s, 1H), 7.92 (s, 1H), 7.71 (d, J=8.2 Hz, 2H), 7.55 (d, J=7.1 Hz, 1H), 7.37 (m, J=7.3 Hz, 2H), 7.31 (d, J=8.2 Hz, 2H), 7.23 (q, J=4.9 Hz, 1H), 5.54 (s, 2H), 3.75 (s, 3H), 3.48 (m, J=6.8 Hz, 1H), 3.05 (s, 6H), 1.06 (d, J=6.8 Hz, 6H).

### Example T-003

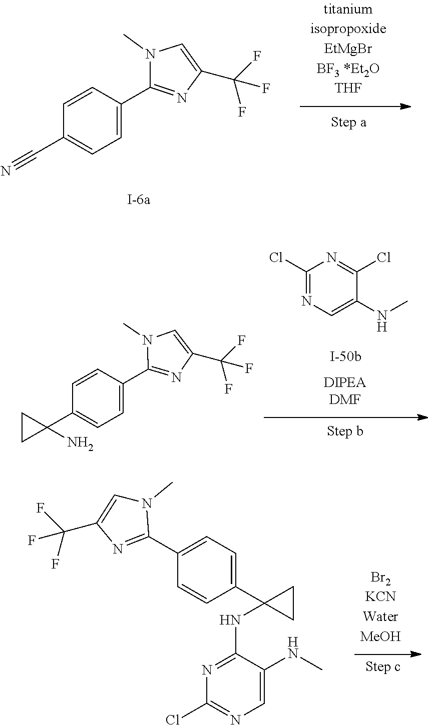

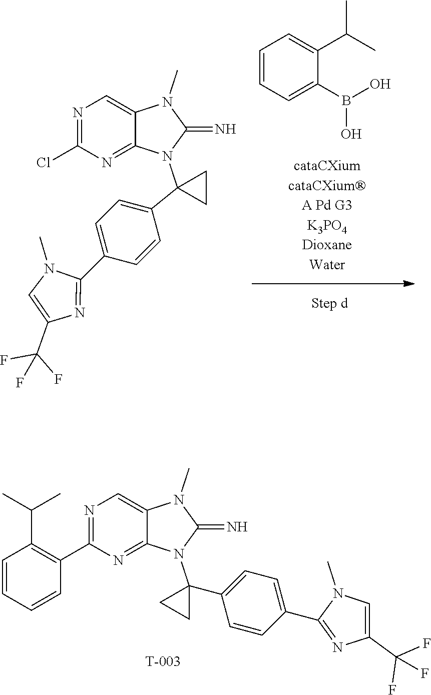 [SMILES: Cn1cc(C(F)(F)F)nc1-c1ccc(C2(n3c(=N)n(C)c4cnc(Cl)nc43)CC2)cc1 ; CC(C)c1ccccc1B(O)O ; CC(C)c1ccccc1-c1ncc2c(n1)n(C1(c3ccc(-c4nc(C(F)(F)F)cn4C)cc3)CC1)c(=N)n2C]

### Step a) 1-[4-[I-methyl-4-(trifluoromethyl)imidazol-2-yl]phenyl]cyclopropanamine

To a mixture of 4-[1-methyl-4-(trifluoromethyl)imidazol-2-yl]benzonitrile (0.80 g, 3.18 mmol) and titanium isopropoxide (905 mg, 3.18 mmol, 948 μL) in THE (10 mL) ethylmagnesium bromide (3.4 M, 1.9 mL, 6.46 mmol) was added dropwise at −80° C. The reaction mixture was stirred for 1 hr. at ambient temperature. Then boron trifluoride diethyl etherate (720 μL, 814 mg, 5.73 mmol) was added and the resulting mixture was stirred at room temperature overnight. The reaction mixture was quenched with 20% aqueous sodium hydroxide (2 mL). The product was extracted with DCM (10 mL×3). The combined organic layers were dried over anhydrous sodium sulfate and concentrated in vacuo. The residue was subjected to flash-column chromatography (SiO₂; gradient MTBE-MeOH) to afford 1-[4-[1-methyl-4-(trifluoromethyl)imidazol-2-yl]phenyl]cyclopropanamine (0.10 g, 360 μmol, 11% yield) as yellow oil which was used in the next steps without further purification.

¹H NMR (400 MHz, CDCl₃) δ 1.05 (m, 2H), 1.17 (m, 2H), 3.76 (s, 3H), 7.31 (s, 1H), 7.40 (m, 2H), 7.58 (m, 2H).

LCMS(ESI): [M+H]⁺ m/z: calc d 282.14; found 282.1.

### Step b) 2-chloro-N5-methyl-N4-[1-[4-[1-methyl-4-(trifluoromethyl)imidazol-2-yl]phenyl]cyclopropyl]pyrimidine-4,5-diamine

1-[4-[1-methyl-4-(trifluoromethyl)imidazol-2-yl]phenyl]cyclopropanamine (0.10 g, 360 μmol), 2,4-dichloro-N-methyl-pyrimidin-5-amine (0.10 g, 560 μmol) and DIPEA (46 mg, 360 μmol, 62 μL) were mixed in DMF (1 mL). The resulting mixture was stirred at 90° C. for 12 hr. At this point, LCMS showed only small conversion. The reaction mixture was heated at 115° C. for 12 h. Then it was cooled to room temperature, diluted with water (3 mL) and extracted with EtOAc (3 mL×2). Combined organic extracts were washed with water (2 mL×3), dried over anhydrous sodium sulfate and concentrated in vacuo to yield 2-chloro-N5-methyl-N4-[1-[4-[1-methyl-4-(trifluoromethyl)imidazol-2-yl]phenyl]cyclopropyl]pyrimidine-4,5-diamine (0.15 g, crude, 24% by LCMS) as a white solid which was used in the next step without further purification.

¹H NMR (400 MHz, DMSO-d₆) δ 1.26 (m, 2H), 1.36 (m, 2H), 2.71 (d, 3H), 3.72 (s, 3H), 5.13 (q, 1H), 7.19-7.28 (m, 3H), 7.57 (d, 2H), 7.72 (s, 1H), 7.86 (s, 1H).

LCMS(ESI): [M+H]⁺ m/z: calcd 423.15; found 423.0.

### Step c) 2-chloro-7-methyl-9-[1-[4-[1-methyl-4-(trifluoromethyl)imidazol-2-yl]phenyl]cyclopropyl]purin-8-imine

A solution of potassium cyanide (46 mg, 710 μmol) in water (0.25 mL) was added at 0° C. to a solution of bromine (113 mg, 710 μmol, 36.6 μL) in MeOH (0.5 mL). The resulting mixture was stirred until discoloration. 2-chloro-N5-methyl-N4-[1-[4-[1-methyl-4-(trifluoromethyl)imidazol-2-yl]phenyl]cyclopropyl]pyrimidine-4,5-diamine (0.15 g, 354.75 mol) in MeOH (0.5 mL) was added and the obtained mixture was stirred for 12 hr. at room temperature. The reaction mixture was quenched with aqueous sodium carbonate until pH≈ 9 and extracted with DCM (3 mL×2). The combined organic layers were dried over anhydrous sodium sulfate and concentrated under reduced pressure to give 2-chloro-7-methyl-9-[1-[4-[1-methyl-4-(trifluoromethyl)imidazol-2-yl]phenyl]cyclopropyl]purin-8-imine (0.10 g, crude, 57% purity by LCMS) as light yellow oil which was used in the next step without further purification.

LCMS(ESI): [M+H]⁺ m/z: calcd 448.14; found 448.2.

### Step d) 2-(2-isopropylphenyl)-7-methyl-9-[1-[4-[1-methyl-4-(trifluoromethyl)imidazol-2-yl]phenyl]cyclopropyl]purin-8-imine

2-chloro-7-methyl-9-[1-[4-[1-methyl-4-(trifluoromethyl)imidazol-2-yl]phenyl]cyclopropyl]purin-8-imine (0.10 g, 220 μmol), (2-isopropylphenyl)boronic acid (73 mg, 447 μmol), cataCXium (4.0 mg, 11 μmol) were mixed in dioxane/water (5:1, 4 mL). The mixture was evacuated and backfilled with argon. CataCXium® A Pd G3 (8.0 mg, 11 μmol) and potassium phosphate tribasic anhydrous (237 mg, 1.12 mmol) were added in an inert atmosphere. The reaction mixture was stirred for 12 hr. at 100° C. The mixture was cooled to room temperature and SiliaMetS© Dimercaptotriazine (100 mg) was added. The obtained mixture was stirred for 3 hr. at room temperature and filtered. The filtrate was directly subjected to HPLC (10-40-55-100% 0-2-12-13.2 min, water—ACN, +0.1% vol. of 25% aq. NH₃, 30 mL/min, column: YMC-Actus Triart C18, 100×20 mm, 5 μm) to afford 2-(2-isopropylphenyl)-7-methyl-9-[1-[4-[1-methyl-4-(trifluoromethyl)imidazol-2-yl]phenyl]cyclopropyl]purin-8-imine (T-003) (13 mg, 25 mol, 11% yield) as an off-white solid.

¹H NMR (600 MHz, DMSO-d₆) δ 0.99-1.13 (m, 6H), 1.45-1.80 (m, 4H), 3.35 (s, 3H), 3.43 (m, 1H), 3.69 (s, 3H), 5.94-6.42 (m, 1H), 7.15-7.21 (m, 3H), 7.32 (t, 1H), 7.36 (d, 1H), 7.47 (d, 1H), 7.54-7.61 (m, 2H), 7.87 (s, 1H), 8.17-8.32 (m, 1H).

LCMS(ESI): [M+H]⁺ m/z: calcd 532.28; found 532.4.

### Example T-076

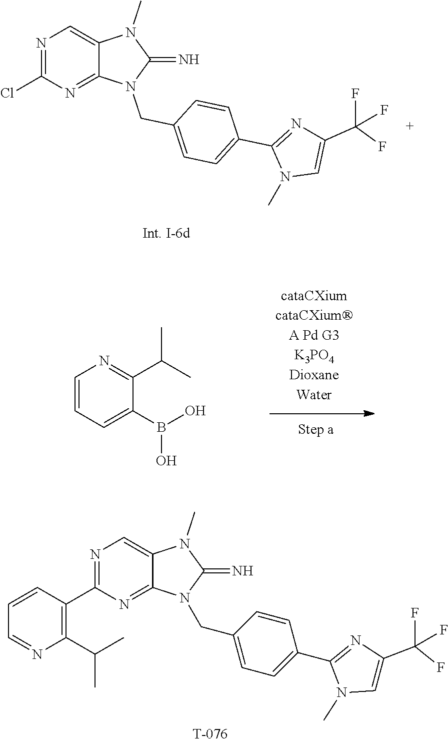 [SMILES: Cn1cc(C(F)(F)F)nc1-c1ccc(Cn2c(=N)n(C)c3cnc(Cl)nc32)cc1 ; CC(C)c1ncccc1B(O)O ; CC(C)c1ncccc1-c1ncc2c(n1)n(Cc1ccc(-c3nc(C(F)(F)F)cn3C)cc1)c(=N)n2C]

### Step a) 2-(2-isopropyl-3-pyridyl)-7-methyl-9-[[4-[I-methyl-4-(trifluoromethyl)imidazol-2-yl]phenyl]methyl]purin-8-imine

To a solution of 2-chloro-7-methyl-9-[[4-[1-methyl-4-(trifluoromethyl)imidazol-2-yl]phenyl]methyl]purin-8-imine (150 mg, 356 μmol) in dioxane/water (5:1, 4 mL) (2-isopropyl-3-pyridyl)boronic acid (117 mg, 711 μmol) and CataCXium (6.4 mg, 18 μmol) were added. The reaction mixture was evacuated and then backfilled with Ar, followed by the addition of CataCXium Pd G3 (13 mg, 18 μmol) and potassium phosphate tribasic anhydrous (377 mg, 1.78 mmol). The reaction mixture was stirred for 12 hr at 100° C. The reaction mixture was cooled then SiliaMetS® Dimercaptotriazine (100 mg) was added. The obtained mixture was stirred for 3 hr. at room temperature then filtered. The filtrate was directly subjected to HPLC (2-10 min 30% water—ACN, +0.1% vol. of 25% aq. NH₃, 30 mL/min, column: YMC-Actus Triart C18, 100×20 mm, 5 μm) to afford 2-(2-isopropyl-3-pyridyl)-7-methyl-9-[[4-[1-methyl-4-(trifluoromethyl)imidazol-2-yl]phenyl]methyl]purin-8-imine (T-076)(13.0 mg, 25.7 μmol, 7.22% yield) as a yellow solid.

¹H NMR (500 MHz, DMSO-d₆) δ 1.11 (d, 6H), 3.34-3.41 (m, 3H), 3.55-3.66 (m, 1H), 3.73 (s, 3H), 5.08-5.24 (m, 2H), 6.49-6.66 (m, 1H), 7.26 (dd, 1H), 7.42-7.51 (m, 2H), 7.63-7.68 (m, 2H), 7.87-7.93 (m, 2H), 8.18-8.30 (m, 1H), 8.54 (dd, 1H).

LCMS(ESI): [M+H]⁺ m/z: calcd 507.25; found 507.2.

### Example T-009

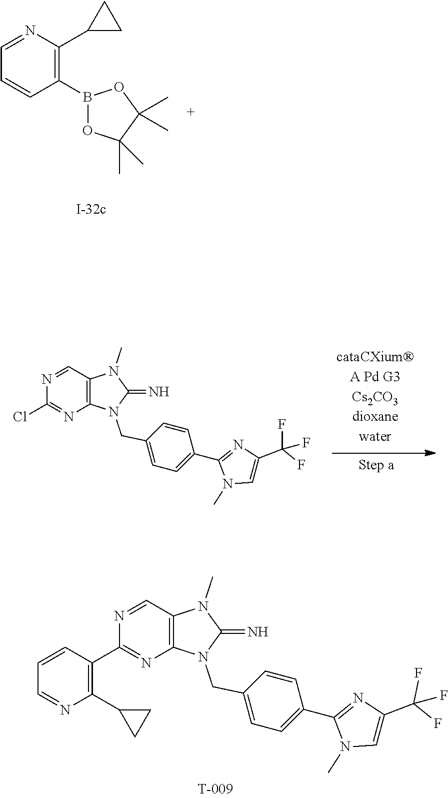 [SMILES: CC1(C)OB(c2cccnc2C2CC2)OC1(C)C ; Cn1cc(C(F)(F)F)nc1-c1ccc(Cn2c(=N)n(C)c3cnc(Cl)nc32)cc1 ; Cn1cc(C(F)(F)F)nc1-c1ccc(Cn2c(=N)n(C)c3cnc(-c4cccnc4C4CC4)nc32)cc1]

### Step a) 2-(2-cyclopropyl-3-pyridyl)-7-methyl-9-[[4-[I-methyl-4-(trifluoromethyl)imidazol-2-yl]phenyl]methyl]purin-8-imine

2-chloro-7-methyl-9-[[4-[1-methyl-4-(trifluoromethyl)imidazol-2-yl]phenyl]methyl]purin-8-imine (150 mg, 356 μmol) was dissolved in dioxane (1 mL) and water (0.1 mL). The reaction mixture was evacuated and then backfilled with argon twice. 2-cyclopropyl-3-(4,4,5,5-tetramethyl-1,3,2-dioxaborolan-2-yl)pyridine (174 mg, 711 μmol), cesium carbonate (348 mg, 1.07 mmol) and cataCXium© A Pd G3 (15.0 mg, 20.6 μmol) were added. The resulting mixture was stirred at 90° C. for 18 hr. The reaction mixture was cooled to room temperature and diluted with methanol (5 mL). SiliaMetS® Dimercaptotriazine (150 mg) was added and the resulting mixture was stirred at room temperature for 8 hr. The mixture was filtered and the filtrate was concentrated in vacuo. The residue was subjected to HPLC (0.5-6.5 min 35-60% water—ACN; flow: 30 mL/min, column: Waters SunFire C18, 100×19 mm, 5 μm) to afford 2-(2-cyclopropyl-3-pyridyl)-7-methyl-9-[[4-[1-methyl-4-(trifluoromethyl)imidazol-2-yl]phenyl]methyl]purin-8-imine (T-009)(46.0 mg, 91.2 μmol, 25.6% yield) as a white solid.

¹H NMR (400 MHz, DMSO-d₆) δ 0.70-0.80 (m, 2H), 0.90-1.00 (m, 2H), 2.72-2.85 (m, 1H), 3.35-3.41 (m, 3H), 3.74 (s, 3H), 5.11-5.30 (m, 2H), 6.50-6.69 (m, 1H), 7.21 (dd, 1H), 7.45-7.56 (m, 2H), 7.62-7.72 (m, 2H), 7.91 (s, 1H), 7.97 (d, 1H), 8.21-8.32 (m, 1H), 8.42 (s, 1H).

LCMS(ESI): [M+H]⁺ m/z: calcd 505.23; found 505.2.

### Example T-087

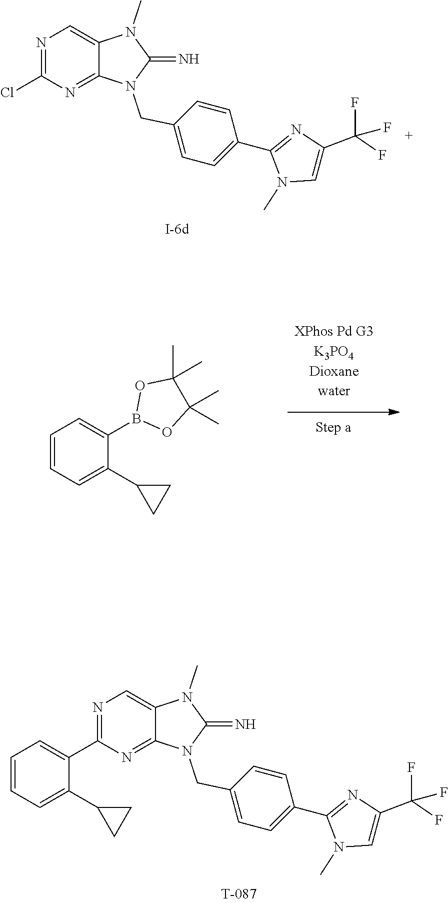 [SMILES: Cn1cc(C(F)(F)F)nc1-c1ccc(Cn2c(=N)n(C)c3cnc(Cl)nc32)cc1 ; CC1(C)OB(c2ccccc2C2CC2)OC1(C)C ; Cn1cc(C(F)(F)F)nc1-c1ccc(Cn2c(=N)n(C)c3cnc(-c4ccccc4C4CC4)nc32)cc1]

### Step a) 2-(2-cyclopropylphenyl)-7-methyl-9-[[4-[1-methyl-4-(trifluoromethyl)imidazol-2-yl]phenyl]methyl]purin-8-imine

2-chloro-7-methyl-9-[[4-[1-methyl-4-(trifluoromethyl)imidazol-2-yl]phenyl]methyl]purin-8-imine (0.15 g, 356 μmol), 2-(2-cyclopropylphenyl)-4,4,5,5-tetramethyl-1,3,2-dioxaborolane (130 mg, 533 μmol), XPhos Pd G3 (15.0 mg, 17.8 μmol) and potassium phosphate tribasic anhydrous (226 mg, 1.07 mmol) were sequentially added to degassed water (0.5 mL) and dioxane (5 mL). The resulting mixture was stirred at 100° C. in an inert atmosphere for 24 hr. The reaction mixture was cooled to room temperature and diluted with EtOAc (20 mL). The obtained mixture was washed with water (5 mL) and brine (5 mL). The organic phase was concentrated under reduced pressure. The residue was subjected to HPLC (0.5-6.5 min 42% water—ACN; flow: 30 mL/min, column: Waters SunFire C18, 100×19 mm, 5 μm) to afford 2-(2-cyclopropylphenyl)-7-methyl-9-[[4-[1-methyl-4-(trifluoromethyl)imidazol-2-yl]phenyl]methyl]purin-8-imine (T-087)(51 mg, 101 μmol, 28.5% yield) as a light-yellow solid.

¹H NMR (400 MHz, DMSO) δ 0.45-0.52 (m, 2H), 0.58-0.67 (m, 2H), 2.32-2.47 (m, 1H), 3.40 (s, 3H), 3.74 (s, 3H), 5.18 (s, 2H), 6.61 (br, 1H), 6.96 (d, 1H), 7.20 (t, 1H), 7.28 (t, 1H), 7.47 (d, 2H), 7.56 (d, 1H), 7.66 (d, 2H), 7.91 (s, 1H), 8.24 (s, 1H).

LCMS(ESI): [M+H]⁺ m/z: calcd 504.24; found 504.2.

### Example T-033

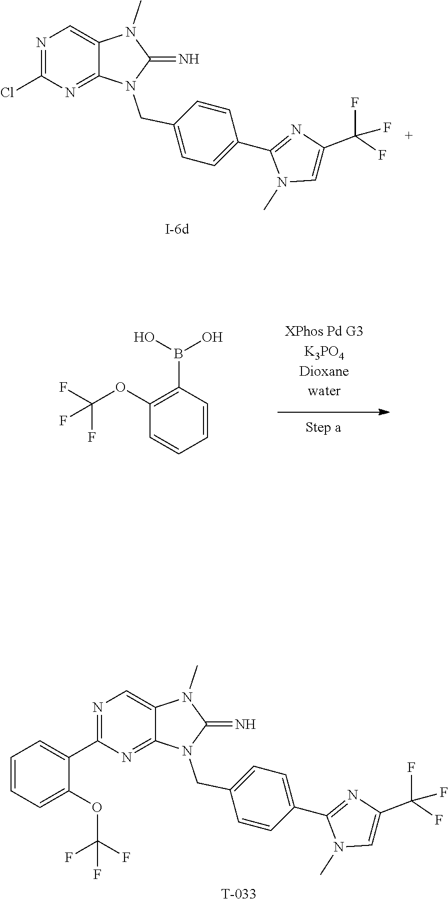 [SMILES: Cn1cc(C(F)(F)F)nc1-c1ccc(Cn2c(=N)n(C)c3cnc(Cl)nc32)cc1 ; OB(O)c1ccccc1OC(F)(F)F ; Cn1cc(C(F)(F)F)nc1-c1ccc(Cn2c(=N)n(C)c3cnc(-c4ccccc4OC(F)(F)F)nc32)cc1]

### Step a) 7-methyl-9-[[4-[I-methyl-4-(trifluoromethyl)imidazol-2-yl]phenyl]methyl]-2-[2-(trifluoromethoxy)phenyl]purin-8-imine (NBK0066-115)

To a mixture of 2-chloro-7-methyl-9-[[4-[1-methyl-4-(trifluoromethyl)imidazol-2-yl]phenyl]methyl]purin-8-imine (90.0 mg, 213 μmol), [2-(trifluoromethoxy)phenyl]boronic acid (87.88 mg, 426.74 μmol) and XPhos Pd G3 (10.67 μmol) in dioxane (10 mL) a solution of potassium phosphate tribasic anhydrous (136 mg, 640 μmol) in water (0.5 mL) was added. The resulting mixture was evacuated and then backfilled with argon. This operation was repeated three times. The resulting mixture was stirred at 90° C. for 16 hr. The reaction mixture was cooled, diluted with water (10 mL) and extracted with EtOAc (2×20 mL). The combined organic layers were washed with water (20 mL) and brine, then dried over anhydrous sodium sulfate and concentrated under reduced pressure. The residue was subjected to HPLC (2-8 min 50-75% water—ACN, +0.1% vol. of 25% aq. NH₃, 30 mL/min, column: Waters SunFire C18, 100×19 mm, 5 μm) to afford 7-methyl-9-[[4-[1-methyl-4-(trifluoromethyl)imidazol-2-yl]phenyl]methyl]-2-[2-(trifluoromethoxy)phenyl]purin-8-imine (T-033)(70.0 mg, 128 μmol, 59.9% yield) as a white solid.

¹H NMR (600 MHz, DMSO-d₆) δ 3.34-3.40 (m, 3H), 3.72 (s, 3H), 5.07-5.25 (m, 2H), 6.54 (br, 1H), 7.43 (d, 1H), 7.44-7.59 (m, 4H), 7.62-7.69 (m, 2H), 7.89 (s, 1H), 7.93 (dd, 1H), 8.25 (br, 1H).

LCMS(ESI): [M+H]⁺ m/z: calcd 548.18; found 548.2.

### Example T-021

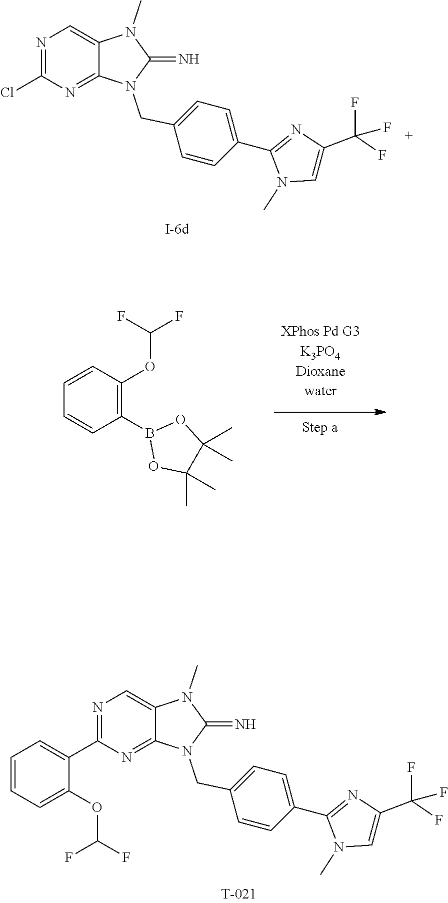 [SMILES: Cn1cc(C(F)(F)F)nc1-c1ccc(Cn2c(=N)n(C)c3cnc(Cl)nc32)cc1 ; CC1(C)OB(c2ccccc2OC(F)F)OC1(C)C ; Cn1cc(C(F)(F)F)nc1-c1ccc(Cn2c(=N)n(C)c3cnc(-c4ccccc4OC(F)F)nc32)cc1]

### Step a) 2-[2-(difluoromethoxy)phenyl]-7-methyl-9-[[4-[1-methyl-4-(trifluoromethyl)imidazol-2-yl]phenyl]methyl]purin-8-imine

2-chloro-7-methyl-9-[[4-[1-methyl-4-(trifluoromethyl)imidazol-2-yl]phenyl]methyl]purin-8-imine (80.0 mg, 190 μmol), 2-[2-(difluoromethoxy)phenyl]-4,4,5,5-tetramethyl-1,3,2-dioxaborolane (102 mg, 379 μmol) and XPhos Pd G3 (8.0 mg. 9.48 μmol) were dissolved in dioxane (8 mL). Then potassium phosphate tribasic anhydrous (121 mg, 567 μmol) in water (0.5 mL) was added. The resulting mixture was evacuated and then backfilled with argon. This operation was repeated three times. The resulting mixture was stirred at 90° C. for 16 hr. The reaction mixture was cooled, diluted with water (10 mL) and extracted with EtOAc (2×20 mL). The combined organic layers were washed with water (20 mL) and brine (20 mL), then dried over anhydrous sodium sulfate and concentrated under reduced pressure. The residue was subjected to IPLC (2-8 min 50-75% water—ACN, +0.1% vol. of 25% aq. NH₃, 30 mL/min; column: Waters SunFire C18, 100×19 mm, 5 μm) to afford 2-[2-(difluoromethoxy)phenyl]-7-methyl-9-[[4-[1-methyl-4-(trifluoromethyl)imidazol-2-yl]phenyl]methyl]purin-8-imine (T-021) (60.0 mg, 113 μmol, 59.8% yield) as a white solid.

¹H NMR (600 MHz, DMSO-d₆) δ 3.33-3.40 (m, 3H), 3.72 (s, 3H), 5.07-5.25 (m, 2H), 6.50 (br, 1H), 7.11 (t, 1H, CHF₂), 7.26 (d, 1H), 7.36 (t, 1H), 7.45-7.54 (m, 3H), 7.61-7.69 (m, 2H), 7.81 (d, 1H), 7.89 (s, 1H), 8.18-8.29 (m, 1H).

LCMS(ESI): [M+H]⁺ m/z: calcd 530.19; found 530.2.

### Example T-071

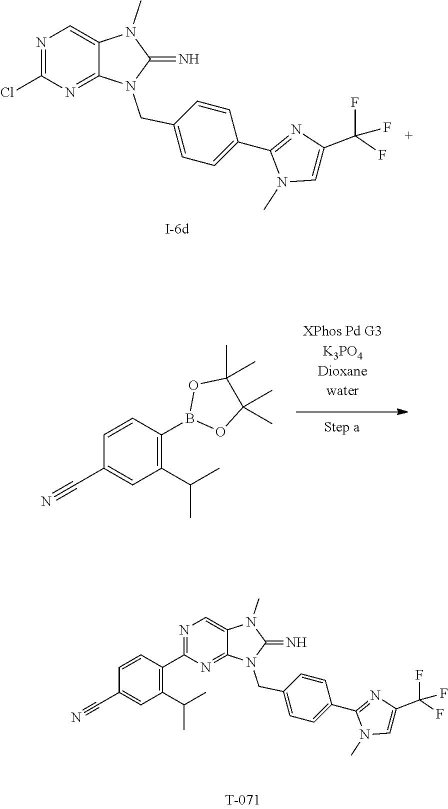 [SMILES: Cn1cc(C(F)(F)F)nc1-c1ccc(Cn2c(=N)n(C)c3cnc(Cl)nc32)cc1 ; CC(C)c1cc(C#N)ccc1B1OC(C)(C)C(C)(C)O1 ; CC(C)c1cc(C#N)ccc1-c1ncc2c(n1)n(Cc1ccc(-c3nc(C(F)(F)F)cn3C)cc1)c(=N)n2C]

### Step a) 4-[8-imino-7-methyl-9-[[4-[I-methyl-4-(trifluoromethyl)imidazol-2-yl]phenyl]methyl]purin-2-yl]-3-isopropyl-benzonitrile

2-chloro-7-methyl-9-[[4-[1-methyl-4-(trifluoromethyl)imidazol-2-yl]phenyl]methyl]purin-8-imine (85 mg, 202 μmol), 3-isopropyl-4-(4,4,5,5-tetramethyl-1,3,2-dioxaborolan-2-yl)benzonitrile (82 mg, 304 μmol), potassium phosphate tribasic anhydrous (128 mg, 605 μmol) and XPhos Pd G3 (8.5 mg, 10.1 μmol) were sequentially added to a mixture of water (2 mL) and dioxane (10 mL). The reaction mixture was evacuated and then backfilled with Ar. The resulting mixture was stirred at 100° C. for 12 hr. The reaction mixture was cooled, diluted with EtOAc (30 mL), washed with water (10 mL) and brine (10 mL). SiliaMetS® Dimercaptotriazine (30 mg) was added to the obtained solution. The obtained mixture was stirred for 30 min and filtered. The filtrate was concentrated under reduce pressure. The residue was subjected to HPLC (0.5-6.5 min 44% water—ACN; flow: 30 mL/min, column: Waters SunFire C18, 100×19 mm, 5 μm) to afford 4-[8-imino-7-methyl-9-[[4-[1-methyl-4-(trifluoromethyl)imidazol-2-yl]phenyl]methyl]purin-2-yl]-3-isopropyl-benzonitrile (T-071)(32.0 mg, 60.3 μmol, 29.9% yield) as a yellow solid.

¹H NMR (500 MHz, DMSO-d₆) δ 1.09 (d, 6H), 3.34-3.40 (m, 3H), 3.43-3.55 (m, 1H), 3.73 (s, 3H), 5.06-5.23 (m, 2H), 6.53-6.67 (m, 1H), 7.38-7.49 (m, 2H), 7.61-7.70 (m, 4H), 7.88 (d, 2H), 8.20-8.29 (m, 1H).

LCMS(ESI): [M+H]⁺ m/z: calcd 531.25; found 531.2.

### Example T-019

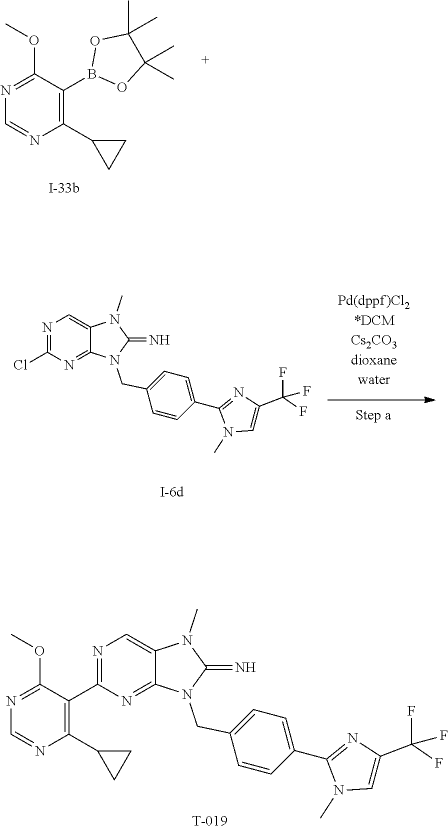 [SMILES: COc1ncnc(C2CC2)c1B1OC(C)(C)C(C)(C)O1 ; Cn1cc(C(F)(F)F)nc1-c1ccc(Cn2c(=N)n(C)c3cnc(Cl)nc32)cc1 ; COc1ncnc(C2CC2)c1-c1ncc2c(n1)n(Cc1ccc(-c3nc(C(F)(F)F)cn3C)cc1)c(=N)n2C]

### Step a) 2-(4-cyclopropyl-6-methoxy-pyrimidin-5-yl)-7-methyl-9-[[4-[I-methyl-4-(trifluoromethyl)imidazol-2-yl]phenyl]methyl]purin-8-imine

To a mixture of 2-chloro-7-methyl-9-[[4-[1-methyl-4-(trifluoromethyl)imidazol-2-yl]phenyl]methyl]purin-8-imine (144 mg, 342 μmol) and 4-cyclopropyl-6-methoxy-5-(4,4,5,5-tetramethyl-1,3,2-dioxaborolan-2-yl)pyrimidine (315 mg, 513 μmol) in dioxane (10 mL) cesium carbonate (335 mg, 1.03 mmol) in water (2 mL) was added. The reaction mixture was evacuated and then backfilled with argon. Bis(diphenylphosphino)ferrocene]dichloropalladium(II)-DCM (28.0 mg, 34.2 μmol) was added in an inert atmosphere. The resulting mixture was stirred at 100° C. for 18 hr. The reaction mixture was cooled to room temperature and concentrated under reduced pressure. The obtained material was diluted with water (5 mL) and EtOAc (10 mL). The organic phase was separated, washed with water (2×5 mL) and filtered through a pad of SiO₂.

The mother liquor was concentrated under reduced pressure. The residue was subjected to IPLC (0-1-6 min 50-50-60% water—methanol, +0.1% vol. of 25% aq. NH₃, 30 mL/min, column: YMC-Actus Triart C18, 100×20 mm, 5 μm) to afford 2-(4-cyclopropyl-6-methoxy-pyrimidin-5-yl)-7-methyl-9-[[4-[1-methyl-4-(trifluoromethyl)imidazol-2-yl]phenyl]methyl]purin-8-imine L-019) (11.0 mg, 20.5 μmol, 6.00% yield) as light-yellow gum which solidified upon freeze-drying into white powder.

¹H NMR (500 MHz, DMSO-d₆) δ 0.72-0.85 (m, 2H), 0.89-1.03 (m, 2H), 1.64-1.73 (m, 1H), 3.32-3.40 (m, 3H), 3.73 (s, 3H), 3.81 (s, 3H), 5.03-5.23 (m, 2H), 6.49-6.65 (m, 1H), 7.48 (d, 2H), 7.64 (d, 2H), 7.90 (s, 1H), 8.14-8.26 (m, 1H), 8.61 (s, 1H).

LCMS(ESI): [M+H]⁺ m/z: calcd 536.23; found 536.2.

### Example T-018

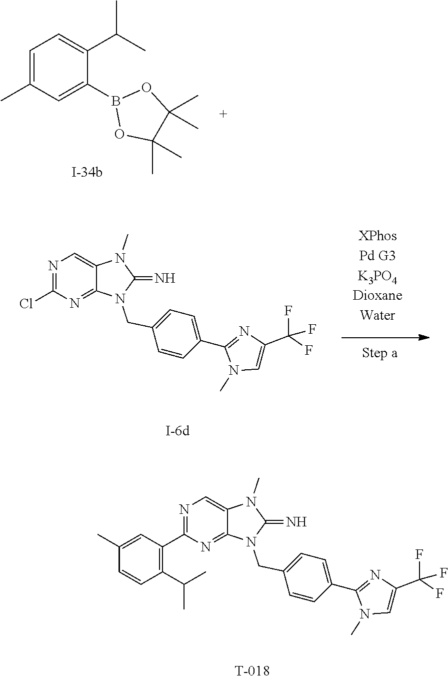

### Step a) 2-(2-isopropyl-5-methyl-phenyl)-7-methyl-9-[[4-[1-methyl-4-(trifluoromethyl)imidazol-2-yl]phenyl]methyl]purin-8-imine

2-(2-isopropyl-5-methyl-phenyl)-4,4,5,5-tetramethyl-1,3,2-dioxaborolane (0.15 g, 576.53 μmol), 2-chloro-7-methyl-9-[[4-[1-methyl-4-(trifluoromethyl)imidazol-2-yl]phenyl]methyl]purin-8-imine (111 mg, 262 μmol) and potassium phosphate tribasic anhydrous (167 mg, 786 μmol) were dissolved in dioxane (8 mL) and water (0.5 mL). The reaction mixture was evacuated and backfilled with argon. XPhosPdG3 (10.0 mg, 13.1 μmol) was added. The resulting mixture was stirred at 90° C. for 18 hr. in an inert atmosphere. The reaction mixture was cooled, diluted with water (10 mL) and extracted with EtOAc (20 mL). The organic layer was washed with water (10 mL) and brine (10 mL), dried over anhydrous sodium sulfate and concentrated in vacuo. The residue was subjected to HPLC (2-8 min 50-75% water—ACN; flow: 30 mL/min, column: Waters SunFire C18, 100×19 mm, 5 μm) to afford 2-(2-isopropyl-5-methyl-phenyl)-7-methyl-9-[[4-[1-methyl-4-(trifluoromethyl)imidazol-2-yl]phenyl]methyl]purin-8-imine (T-018)(16.0 mg, 30.8 μmol, 11.8% yield) as a yellow solid which solidified upon freeze-drying into white powder.

¹H NMR (600 MHz, DMSO-d₆) δ 1.02-1.08 (m, 6H), 2.27 (s, 3H), 3.34-3.40 (m, 4H), 3.72 (s, 3H), 5.06-5.23 (m, 2H), 6.43-6.54 (m, 1H), 7.15 (d, 1H), 7.22-7.28 (m, 2H), 7.40-7.48 (m, 2H), 7.62-7.67 (m, 2H), 7.89 (s, 1H), 8.16-8.24 (m, 1H).

LCMS(ESI): [M+H]⁺ m/z: calcd 520.28; found 520.2; Rt=1.22.

### Example T-015

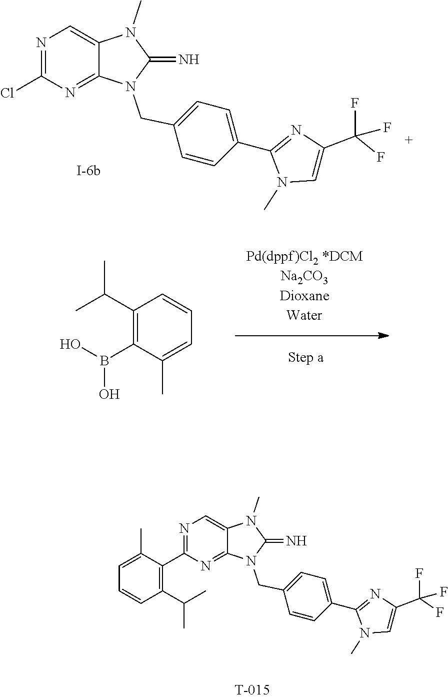 [SMILES: Cn1cc(C(F)(F)F)nc1-c1ccc(Cn2c(=N)n(C)c3cnc(Cl)nc32)cc1 ; Cc1cccc(C(C)C)c1B(O)O ; Cc1cccc(C(C)C)c1-c1ncc2c(n1)n(Cc1ccc(-c3nc(C(F)(F)F)cn3C)cc1)c(=N)n2C]

### Step a) 2-(2-isopropyl-6-methyl-phenyl)-7-methyl-9-[[4-[1-methyl-4-(trifluoromethyl)imidazol-2-yl]phenyl]methyl]purin-8-imine

2-chloro-7-methyl-9-[[4-[1-methyl-4-(trifluoromethyl)imidazol-2-yl]phenyl]methyl]purin-8-imine (100 mg, 237 μmol), (2-isopropyl-6-methyl-phenyl)boronic acid (127 mg, 711 μmol) and bis(diphenylphosphino)ferrocene]dichloropalladium(II)-DCM (20.0 mg, 11.9 μmol) were mixed in dioxane (8 mL) in an inert atmosphere. Then potassium carbonate (98 mg, 711 μmol) in water (0.5 mL) was added. The resulting mixture was evacuated and then backfilled with argon. This operation was repeated three times. The resulting mixture was stirred at 90° C. for 72 hr. An aliquot showed about 20% conversion. Then 1 equivalent of (2-isopropyl-6-methyl-phenyl)boronic (42 mg) was added to the reaction mixture and the mixture was stirred at 90° C. for 48 h. The reaction mixture was cooled, diluted with water (15 mL) and extracted with EtOAc (20 mL). The organic layer was washed with water (10 mL) and brine (10 mL), dried over anhydrous sodium sulfate and concentrated in vacuo. The residue was subjected HPLC (2-8 min 50-75% water—ACN, +0.1% vol. of 25% aq. NH₃, 30 mL/min, column: Waters SunFire C18, 100×19 mm, 5 μm) to afford 2-(2-isopropyl-6-methyl-phenyl)-7-methyl-9-[[4-[1-methyl-4-(trifluoromethyl)imidazol-2-yl]phenyl]methyl]purin-8-imine (T-015) (10.0 mg, 19.3 μmol, 8.12% yield) as a light-yellow solid which solidified upon freeze-drying into white powder.

¹H NMR (600 MHz, DMSO-d₆) δ 0.98 (d, 6H), 1.89 (s, 3H), 3.36-3.41 (m, 4H), 3.71 (s, 3H), 5.12 (br, 2H), 6.13-6.82 (m, 1H), 7.03 (d, 1H), 7.16 (d, 1H), 7.23 (t, 1H), 7.42 (d, 2H), 7.62 (d, 2H), 7.89 (s, 1H), 8.20 (s, 1H).

LCMS(ESI): [M+H]⁺ m/z: calcd 520.28; found 520.2.

### Example T-070

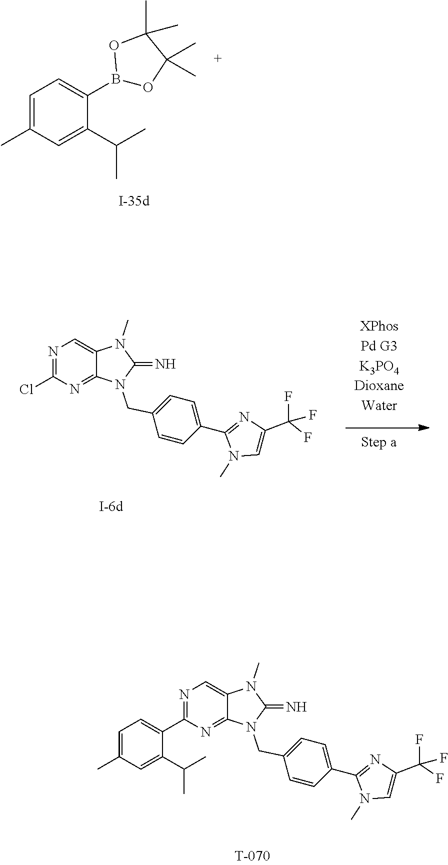

### Step a) 2-(2-isopropyl-4-methyl-phenyl)-7-methyl-9-[[4-[1-methyl-4-(trifluoromethyl)imidazol-2-yl]phenyl]methyl]purin-8-imine

2-(2-isopropyl-4-methyl-phenyl)-4,4,5,5-tetramethyl-1,3,2-dioxaborolane (0.18 g, 692 μmol), 2-chloro-7-methyl-9-[[4-[1-methyl-4-(trifluoromethyl)imidazol-2-yl]phenyl]methyl]purin-8-imine (146 mg, 346 μmol) and potassium phosphate tribasic anhydrous (220 mg, 1.04 mmol) were mixed in dioxane (8 mL) and water (0.5 mL) in an inert atmosphere. The resulting mixture was evacuated and backfilled with argon. XPhos Pd G3 (14.6 mg, 17.30 μmol) was added. The resulting mixture was stirred at 90° C. for 16 hr. The reaction mixture was cooled, diluted with water (15 mL) and extracted with EtOAc (25 mL). The organic layer was washed with water (10 mL) and brine (10 mL), dried over anhydrous sodium sulfate and concentrated in vacuo. The final compound was purified by HPLC (2-8 min 50-75% water -methanol, +0.1% vol. of 25% aq. NH₃, 30 mL/min, column: Waters SunFire C18, 100×19 mm, 5 μm) to afford 2-(2-isopropyl-4-methyl-phenyl)-7-methyl-9-[[4-[1-methyl-4-(trifluoromethyl)imidazol-2-yl]phenyl]methyl]purin-8-imine (T-070) (80.0 mg, 154 μmol, 44.5% yield) as a light-yellow solid which solidified upon freeze-drying into white powder.

¹H NMR (500 MHz, DMSO-d₆) δ 1.07 (d, 6H), 2.31 (s, 3H), 3.36 (s, 3H), 3.43-3.52 (m, 1H), 3.73 (s, 3H), 5.07-5.25 (m, 2H), 6.40-6.55 (m, 1H), 7.01 (d, 1H), 7.18 (s, 1H), 7.39 (d, 1H), 7.46 (d, 2H), 7.65 (d, 2H), 7.89 (s, 1H), 8.19 (br, 1H).

LCMS(ESI): [M+H]⁺ m/z: calcd 520.28; found 520.0; Rt=1.29.

### Example T-002

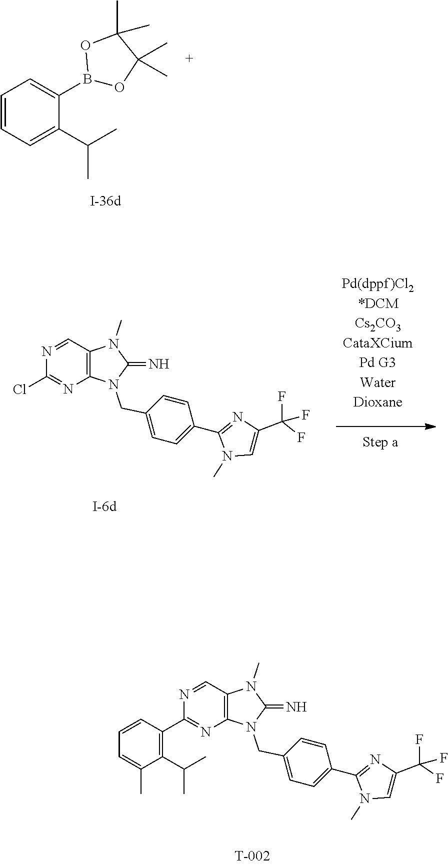 [SMILES: CC(C)c1ccccc1B1OC(C)(C)C(C)(C)O1 ; Cn1cc(C(F)(F)F)nc1-c1ccc(Cn2c(=N)n(C)c3cnc(Cl)nc32)cc1 ; Cc1cccc(-c2ncc3c(n2)n(Cc2ccc(-c4nc(C(F)(F)F)cn4C)cc2)c(=N)n3C)c1C(C)C]

### Step a) 2-(2-isopropyl-3-methyl-phenyl)-7-methyl-9-[[4-[1-methyl-4-(trifluoromethyl)imidazol-2-yl]phenyl]methyl]purin-8-imine

A mixture of water (1 mL) and dioxane (5 mL) was evacuated and backfilled with argon, then 2-(2-isopropyl-3-methyl-phenyl)-4,4,5,5-tetramethyl-1,3,2-dioxaborolane (93 mg, 356 μmol), 2-chloro-7-methyl-9-[[4-[1-methyl-4-(trifluoromethyl)imidazol-2-yl]phenyl]methyl]purin-8-imine (100 mg, 237 μmol), bis(diphenylphosphino)ferrocene]dichloropalladium(II)-DCM (19 mg, 23.7 μmol) and cesium carbonate (154 mg, 474 μmol) were added in an inert atmosphere. The reaction mixture was stirred for 14 hr. at 100° C. LCMS of aliquot showed only traces of product. CataCXium® A Pd G3 (17.3 mg, 23.7 μmol) was added and the resulting mixture was stirred for 16 hr. at 100° C. The reaction mixture was cooled and concentrated in vacuo. The residue was diluted with H₂O (5 mL) and extracted with EtOAc (3×20 mL). The combined organic layers were dried over anhydrous sodium sulfate and concentrated in vacuo. The residue was purified by HPLC (2-8 min 0-65% water+FA (0.1% vol.)—ACN+FA (0.1% vol.); flow: 30 mL/min, column: Waters SunFire C18, 100×19 mm, 5 μm) to afford 2-(2-isopropyl-3-methyl-phenyl)-7-methyl-9-[[4-[1-methyl-4-(trifluoromethyl)imidazol-2-yl]phenyl]methyl]purin-8-imine (T-002)(5.8 mg, 11.2 μmol, 4.71% yield) as a light-yellow solid.

¹H NMR (600 MHz, DMSO-d₆) δ 1.13 (d, 6H), 2.43 (s, 3H), 3.18-3.26 (m, 1H), 3.39 (s, 3H), 3.76 (s, 3H), 5.13 (s, 2H), 7.02-7.06 (m, 2H), 7.06-7.10 (m, 1H), 7.51 (d, 2H), 7.60 (d, 2H), 7.65 (s, 1H), 8.00-8.08 (m, 1H), 8.74-8.96 (m, 1H). LCMS(ESI): [M+H]⁺ m/z: calcd 520.28; found 520.4.

### Example T-082

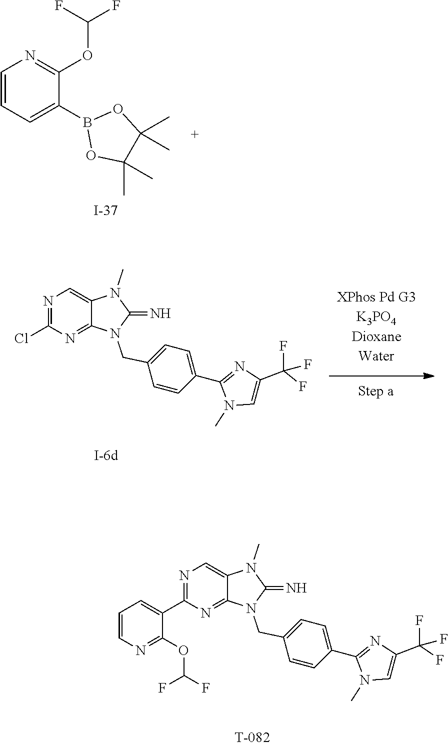

### Step a) 2-[2-(difluoromethoxy)-3-pyridyl]-7-methyl-9-[[4-[I-methyl-4-(trifluoromethyl)imidazol-2-yl]phenyl]methyl]purin-8-imine

2-(difluoromethoxy)-3-(4,4,5,5-tetramethyl-1,3,2-dioxaborolan-2-yl)pyridine (0.20 g, 738 mol), 2-chloro-7-methyl-9-[[4-[1-methyl-4-(trifluoromethyl)imidazol-2-yl]phenyl]methyl]purin-8-imine (156 mg, 369 μmol) and potassium phosphate tribasic anhydrous (235 mg, 1.11 mmol) were mixed in dioxane (8 mL) and water (0.5 mL). The resulting mixture was evacuated and then backfilled with argon. XPhos Pd G3 (10 mg, 18.5 μmol) was added. The resulting mixture was stirred at 90° C. for 18 hr. The reaction mixture was cooled, diluted with water (10 mL) and extracted with EtOAc (20 mL×2). The combined organic layers were washed with water (20 mL) and brine (20 mL), dried over anhydrous sodium sulfate and concentrated under reduced pressure. The residue was subjected HPLC (2-8 min 50-75% water—ACN, +0.1% vol. of 25% aq. NH₃, 30 mL/min, column: Waters SunFire C18, 100×19 mm, 5 μm) to afford 2-[2-(difluoromethoxy)-3-pyridyl]-7-methyl-9-[[4-[1-methyl-4-(trifluoromethyl)imidazol-2-yl]phenyl]methyl]purin-8-imine (T-082)(140 mg, 264 μmol, 71.5% yield) as a light-yellow solid.

¹H NMR (600 MHz, DMSO-d₆) δ 3.33-3.38 (m, 3H), 3.73 (s, 3H), 5.06-5.23 (m, 2H), 6.52-6.63 (m, 1H), 7.38 (dd, 1H), 7.55-7.79 (m, 5H), 7.89 (s, 1H), 8.20-8.29 (m, 2H), 8.31 (dd, 1H).

LCMS(ESI): [M+H]⁺ m/z: calcd 531.18; found 531.2.

### Example T-027

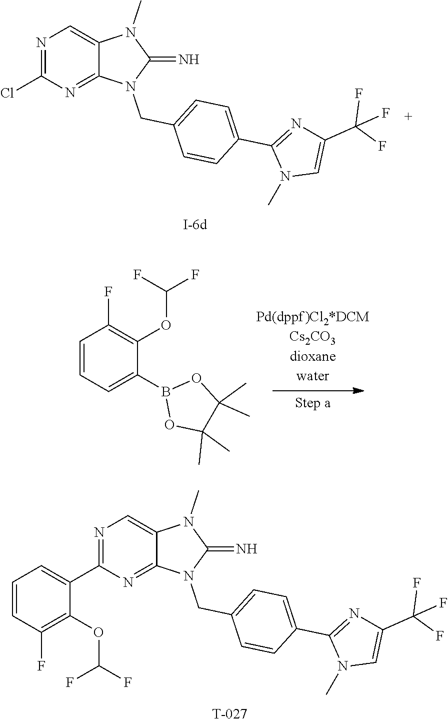

### Step a) 2-[2-(difluoromethoxy)-3-fluoro-phenyl]-7-methyl-9-[[4-[I-methyl-4-(trifluoromethyl)imidazol-2-yl]phenyl]methyl]purin-8-imine

To a mixture of 2-chloro-7-methyl-9-[[4-[1-methyl-4-(trifluoromethyl)imidazol-2-yl]phenyl]methyl]purin-8-imine (175 mg, 415 μmol) in dioxane (4 mL) and water (1 mL) 2-[2-(difluoromethoxy)-3-fluoro-phenyl]-4,4,5,5-tetramethyl-1,3,2-dioxaborolane (167 mg, 581 μmol), cesium carbonate (406 mg, 1.24 mmol) and bis(diphenylphosphino)ferrocene]dichloropalladium(II)-DCM (16.9 mg, 20.7 μmol) were sequentially added in an inert atmosphere. The resulting mixture was stirred at 90° C. overnight. The reaction mixture was cooled to room temperature and concentrated under reduced pressure. The residue was diluted with EtOAc (20 mL) and washed with brine (10 mL), dried over anhydrous sodium sulfate and concentrated in vacuo. The residue was subjected to HPLC (0-1-6 min 35-35-80% water—ACN, +0.1% vol. of 25% aq. NH₃, 30 mL/min, column: YMC-Actus Triart C18, 100×20 mm, 5 μm) to give a desired product (30.4 mg, purity 85%).

The obtained material was further purified by HPLC (0-5 min 50-75% water—methanol, +0.1% vol. of 25% aq. NH₃, 30 mL/min, column: YMC-Actus Triart C18, 100×20 mm, 5 μm) to give 2-[2-(difluoromethoxy)-3-fluoro-phenyl]-7-methyl-9-[[4-[1-methyl-4-(trifluoromethyl)imidazol-2-yl]phenyl]methyl]purin-8-imine (T-027)(19.0 mg, 34.7 μmol, 8.37%) as a brown solid.

¹H NMR (600 MHz, DMSO-d₆) δ 3.33-3.39 (m, 3H), 3.73 (s, 3H), 5.10-5.25 (m, 2H), 6.52-6.59 (m, 1H), 7.10 (t, 1H, CHF₂), 7.38-7.46 (m, 2H), 7.46-7.52 (m, 2H), 7.62-7.69 (m, 2H), 7.72 (d, 1H), 7.89 (s, 1H), 8.19-8.29 (m, 1H).

LCMS(ESI): [M+H]⁺ m/z: calcd 548.18; found 548.0.

### Example T-053

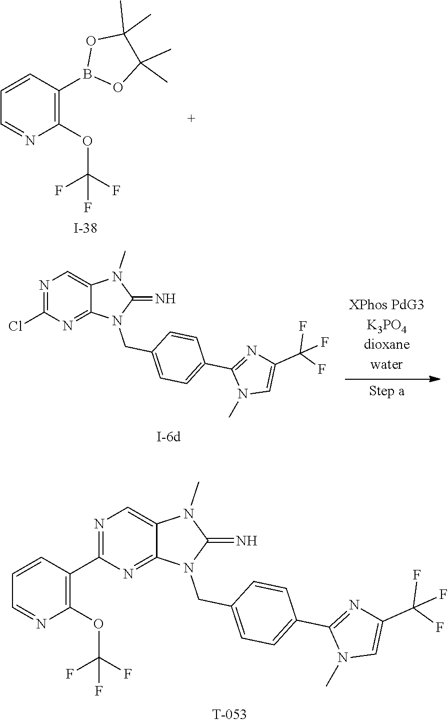

### Step a) 7-methyl-9-[[4-[I-methyl-4-(trifluoromethyl)imidazol-2-yl]phenyl]methyl]-2-[2-(trifluoromethoxy)-3-pyridyl]purin-8-imine

2-chloro-7-methyl-9-[[4-[1-methyl-4-(trifluoromethyl)imidazol-2-yl]phenyl]methyl]purin-8-imine (0.12 g, 284 μmol), 3-(4,4,5,5-tetramethyl-1,3,2-dioxaborolan-2-yl)-2-(trifluoromethoxy)pyridine (411 mg, 711 μmol), potassium phosphate tribasic anhydrous (211 mg, 996 μmol) and XPhos Pd G3 (12.0 mg, 14.2 μmol) were sequentially added to a degassed mixture of water (2 mL) and dioxane (10 mL). The resulting mixture was stirred at 100° C. for 24 hr. in an inert atmosphere. The reaction mixture was cooled, diluted with EtOAc (20 mL), washed with water (10 mL) and brine (10 mL). The organic layer was separated, and then SiliaMetS© Dimercaptotriazine (30 mg) was added, the mixture was stirred for 30 min and filtered. The filtrate was concentrated under reduced pressure. The residue was purified by HPLC (0.5-6.5 min 45% water—ACN; flow: 30 mL/min, column: Waters SunFire C18, 100×19 mm, 5 μm) to afford (T-053)(89 mg, 57.0% yield) as a light-yellow solid.

¹H NMR (400 MHz, DMSO-d₆) δ 3.33-3.43 (m, 3H), 3.74 (s, 3H), 5.08-5.26 (m, 2H), 6.56-6.68 (m, 1H), 7.48-7.60 (m, 3H), 7.66 (d, 2H), 7.91 (s, 1H), 8.24-8.33 (m, 1H), 8.35-8.45 (m, 2H).

LCMS(ESI): [M+H]⁺ m/z: calcd 549.17; found 549.2; Rt=2.77.

### Example T-001

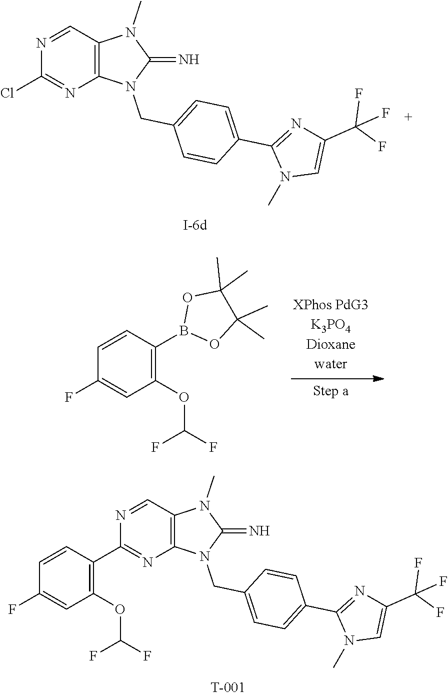

### Step a) 2-[2-(difluoromethoxy)-4-fluoro-phenyl]-7-methyl-9-[[4-[I-methyl-4-(trifluoromethyl)imidazol-2-yl]phenyl]methyl]purin-8-imine

2-chloro-7-methyl-9-[[4-[1-methyl-4-(trifluoromethyl)imidazol-2-yl]phenyl]methyl]purin-8-imine (100 mg, 237 μmol) was dissolved in dioxane (3 mL) and water (0.1 mL). The obtained mixture was evacuated and then backfilled with argon twice. 2-[2-(difluoromethoxy)-4-fluoro-phenyl]-4,4,5,5-tetramethyl-1,3,2-dioxaborolane (137 mg, 474 μmol), XPhos Pd G3 (10.0 mg, 11.9 μmol) and potassium phosphate tribasic anhydrous (151 mg, 711 μmol) were added. The resulting mixture was stirred at 90° C. for 18 hr. The reaction mixture was cooled to room temperature and diluted with EtOAc (10 mL). To the obtained mixture SiliaMetS© Dimercaptotriazine (100 mg) was added and the mixture was stirred for 1 hr. The resulting mixture was dried over anhydrous sodium sulfate Na₂SO₄, filtered and concentrated in vacuo. The residue was subjected HPLC (0.5-6.5 min 45-70% water—ACN, +0.1% vol. of 25% aq. NH₃, 30 mL/min, column: Xbridge 100×19 mm, 5 μm (R)) to afford 2-[2-(difluoromethoxy)-4-fluoro-phenyl]-7-methyl-9-[[4-[1-methyl-4-(trifluoromethyl)imidazol-2-yl]phenyl]methyl]purin-8-imine (T-001) (70.0 mg, 54% yield) as a brown solid.

¹H NMR (400 MHz, DMSO-d₆) δ 3.34-3.40 (m, 3H), 3.74 (s, 3H), 5.11-5.24 (m, 2H), 6.52-6.56 (m, 1H), 7.01-7.40 (m, 3H), 7.48-7.55 (m, 2H), 7.64-7.70 (m, 2H), 7.86-7.94 (m, 2H), 8.19-8.29 (m, 1H).

LCMS(ESI): [M+H]⁺ m/z: calcd 548.18; found 548.2.

### Example T-038

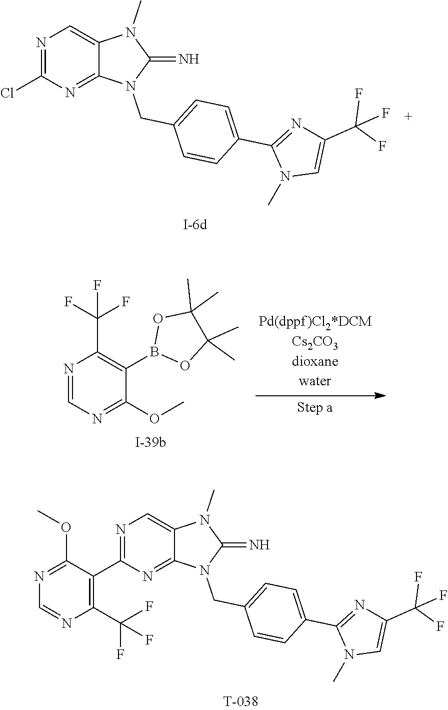 [SMILES: Cn1cc(C(F)(F)F)nc1-c1ccc(Cn2c(=N)n(C)c3cnc(Cl)nc32)cc1 ; COc1ncnc(C(F)(F)F)c1B1OC(C)(C)C(C)(C)O1 ; COc1ncnc(C(F)(F)F)c1-c1ncc2c(n1)n(Cc1ccc(-c3nc(C(F)(F)F)cn3C)cc1)c(=N)n2C]

### Step a) 2-[4-methoxy-6-(trifluoromethyl)pyrimidin-5-yl]-7-methyl-9-[[4-[I-methyl-4-(trifluoromethyl)imidazol-2-yl]phenyl]methyl]purin-8-imine

To a mixture of 2-chloro-7-methyl-9-[[4-[1-methyl-4-(trifluoromethyl)imidazol-2-yl]phenyl]methyl]purin-8-imine (83 mg, 197 μmol), 4-methoxy-5-(4,4,5,5-tetramethyl-1,3,2-dioxaborolan-2-yl)-6-(trifluoromethyl)pyrimidine (200 mg, 197 μmol) and cesium carbonate (193 mg, 592 μmol) in water (1 mL) and dioxane (6 mL) bis(diphenylphosphino)ferrocene]dichloropalladium(II)-DCM (16 mg, 20 μmol) was added under an inert atmosphere. The reaction mixture was stirred at 90° C. for 18 hr. The reaction mixture was cooled to room temperature and concentrated under reduced pressure. The obtained residue was diluted with water (10 mL) and EtOAc (20 mL). The organic phase was separated, washed with water (2×10 mL) and filtered through a pad of SiO₂. The filtrate was concentrated under reduced pressure. The residue was subjected to HPLC (gradient elution 30-45% water—ACN, +0.1% vol. of 25% aq. NH₃, 30 mL/min, column: YMC-Actus Triart C18, 100×20 mm, 5 μm) to afford 2-[4-methoxy-6-(trifluoromethyl)pyrimidin-5-yl]-7-methyl-9-[[4-[1-methyl-4-(trifluoromethyl)imidazol-2-yl]phenyl]methyl]purin-8-imine (T-038)(3.2 mg, 3% yield) as brown gum which solidified upon freeze-drying.

¹H NMR (500 MHz, DMSO-d₆) δ 3.38 (s, 3H), 3.76 (s, 3H), 3.99 (s, 3H), 5.10 (br, 2H), 6.35 (br, 1H), 7.40-7.76 (m, 6H), 8.09 (s, 1H), 8.91 (s, 1H).

LCMS(ESI): [M+H]⁺ m/z: calcd 564.18; found 564.0; Rt=2.45.

### Example T-011

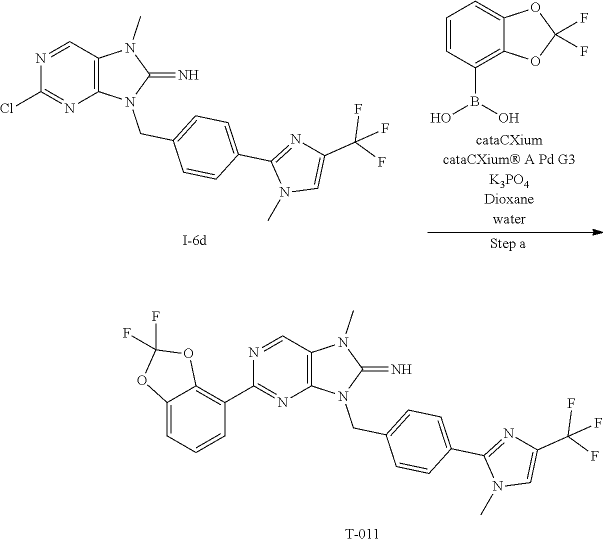 [SMILES: Cn1cc(C(F)(F)F)nc1-c1ccc(Cn2c(=N)n(C)c3cnc(Cl)nc32)cc1 ; OB(O)c1cccc2c1OC(F)(F)O2 ; Cn1cc(C(F)(F)F)nc1-c1ccc(Cn2c(=N)n(C)c3cnc(-c4cccc5c4OC(F)(F)O5)nc32)cc1]

### Step a) 2-(2,2-difluoro-1,3-benzodioxol-4-yl)-7-methyl-9-[[4-[1-methyl-4-(trifluoromethyl)imidazol-2-yl]phenyl]methyl]purin-8-imine

2-chloro-7-methyl-9-[[4-[1-methyl-4-(trifluoromethyl)imidazol-2-yl]phenyl]methyl]purin-8-imine (70 mg, 166 μmol), (2,2-difluoro-1,3-benzodioxol-4-yl)boronic acid (101 mg, 498 μmol) and potassium phosphate tribasic anhydrous (106 mg, 498 μmol) were mixed in dioxane and water (5/1, 5 mL). The obtained mixture was evacuated and then backfilled with argon, followed by the addition of CataCXium (3.0 mg, 8.3 μmol) and cataCXium® Pd G3 (6.0 mg, 8.3 μmol). The resulting mixture was stirred for 12 hr. at 90° C. The reaction mixture was cooled to room temperature and SiliaMetS® Dimercaptotriazine (100 mg) was added. The obtained mixture was stirred for 3 hr. then filtered and subjected to HPLC (2-10 min 50-100% water—methanol, +0.1% vol. of 25% aq. NH₃, 30 mL/min, column: Waters SunFire C18, 100×19 mm, 5 μm) to afford 2-(2,2-difluoro-1,3-benzodioxol-4-yl)-7-methyl-9-[[4-[1-methyl-4-(trifluoromethyl)imidazol-2-yl]phenyl]methyl]purin-8-imine (T-011 (12.0 mg, 22.1 μmol, 13.3% yield) as a brown solid.

¹H NMR (400 MHz, DMSO-d₆) δ 3.33-3.40 (m, 3H), 3.74 (s, 3H), 5.10-5.14 (m, 2H), 6.60-6.77 (m, 1H), 7.31 (t, 1H), 7.46 (d, 1H), 7.58-7.70 (m, 4H), 7.91 (s, 1H), 8.01 (d, 1H), 8.21-8.30 (m, 1H).

LCMS(ESI): [M+H]⁺ m/z: calcd 544.16; found 544.0.

### Example T-039

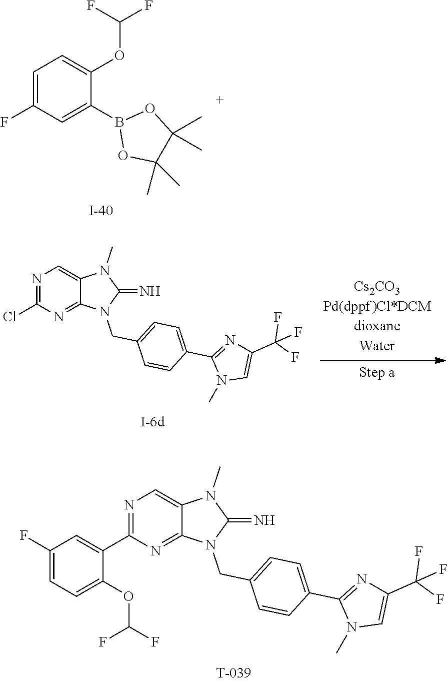

### Step a) 2-[2-(difluoromethoxy)-5-fluoro-phenyl]-7-methyl-9-[[4-[I-methyl-4-(trifluoromethyl)imidazol-2-yl]phenyl]methyl]purin-8-imine

To a solution of 2-chloro-7-methyl-9-[[4-[1-methyl-4-(trifluoromethyl)imidazol-2-yl]phenyl]methyl]purin-8-imine (120 mg, 284 μmol) in dioxane (4 mL) and water (1 mL) 2-[2-(difluoromethoxy)-5-fluoro-phenyl]-4,4,5,5-tetramethyl-1,3,2-dioxaborolane (115 mg, 398 μmol), cesium carbonate (278 mg, 853 μmol) and bis(diphenylphosphino)ferrocene]dichloropalladium(II)-DCM (11.6 mg, 14.2 μmol) were sequentially added in an inert atmosphere. The resulting mixture was stirred at 90° C. for 16 hr. The reaction mixture was cooled and concentrated under reduced pressure. The residue was diluted with EtOAc (20 mL), washed with brine (10 mL), dried over anhydrous sodium sulfate and concentered in vacuo. The residue was subjected to HPLC (0-5 min 50-75% water -methanol, +0.1% vol. of 25% aq. NH₃, 30 mL/min, column: YMC-Actus Triart C18, 100×20 mm, 5 μm) to afford 2-[2-(difluoromethoxy)-5-fluoro-phenyl]-7-methyl-9-[[4-[1-methyl-4-(trifluoromethyl)imidazol-2-yl]phenyl]methyl]purin-8-imine (T-039)(6.5 mg, 11.9 μmol, 4.17%) as a light-yellow solid.

¹H NMR (500 MHz, DMSO-d₆) δ 3.34-3.41 (m, 3H), 3.72 (s, 3H), 5.06-5.28 (m, 2H), 6.56 (s, 1H), 7.07 (t, 1H, CHF₂), 7.28-7.37 (m, 2H), 7.45-7.57 (m, 2H), 7.60-7.69 (m, 3H), 7.89 (s, 1H), 8.17-8.33 (m, 1H).

LCMS(ESI): [M+H]⁺ m/z: calcd 548.18; found 548.0; Rt=3.07.

### Example T-079

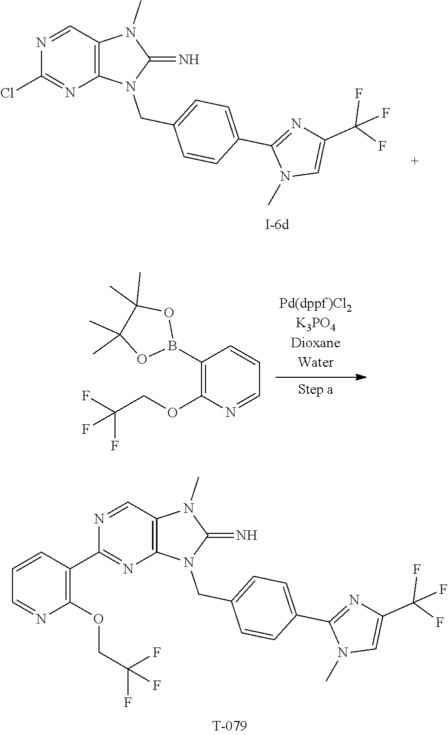 [SMILES: Cn1cc(C(F)(F)F)nc1-c1ccc(Cn2c(=N)n(C)c3cnc(Cl)nc32)cc1 ; CC1(C)OB(c2cccnc2OCC(F)(F)F)OC1(C)C ; Cn1cc(C(F)(F)F)nc1-c1ccc(Cn2c(=N)n(C)c3cnc(-c4cccnc4OCC(F)(F)F)nc32)cc1]

### Step a) 7-methyl-9-[[4-[I-methyl-4-(trifluoromethyl)imidazol-2-yl]phenyl]methyl]-2-[2-(2,2,2-trifluoroethoxy)-3-pyridyl]purin-8-imine

To a solution of 2-chloro-7-methyl-9-[[4-[1-methyl-4-(trifluoromethyl)imidazol-2-yl]phenyl]methyl]purin-8-imine (70 mg, 166 μmol) in dioxane/water (5/1, 5 mL) 3-(4,4,5,5-tetramethyl-1,3,2-dioxaborolan-2-yl)-2-(2,2,2-trifluoroethoxy)pyridine (100 mg, 332 μmol) and potassium phosphate tribasic (88 mg, 415 μmol) were added. The resulting mixture was evacuated and then backfilled with Ar. Bis(diphenylphosphino)ferrocene]dichloropalladium(II)-DCM (9.5 mg, 12 μmol) was added in an argon atmosphere. The resulting mixture was stirred at 90° C. for 48 hr. The reaction mixture was cooled and SiliaMetS© Dimercaptotriazine (100 mg) was added. The obtained mixture was stirred for 3 h, filtered and purified by HPLC (2-10 min 30-55% water—ACN, +0.1% vol. of 25% aq. NH₃, 30 mL/min, column: YMC-Actus Triart C18, 100×20 mm, 5 μm) to afford 7-methyl-9-[[4-[1-methyl-4-(trifluoromethyl)imidazol-2-yl]phenyl]methyl]-2-[2-(2,2,2-trifluoroethoxy)-3-pyridyl]purin-8-imine (T-079)(12.0 mg, 21.3 mol, 12.9% yield) as brown oil which solidified upon freeze drying into light-brown powder.

¹H NMR (600 MHz, DMSO-d₆) δ 3.28-3.39 (m, 3H), 3.72 (s, 3H), 5.01 (q, 2H), 5.06-5.22 (m, 2H), 6.48 (s, 1H), 7.21 (dd, 1H), 7.49 (d, 2H), 7.61-7.68 (m, 2H), 7.89 (s, 1H), 8.12-8.16 (m, 1H), 8.19-8.29 (m, 2H).

LCMS(ESI): [M+H]⁺ m/z: calcd 563.19; found 563.0; Rt=3.08.

### Example T-036

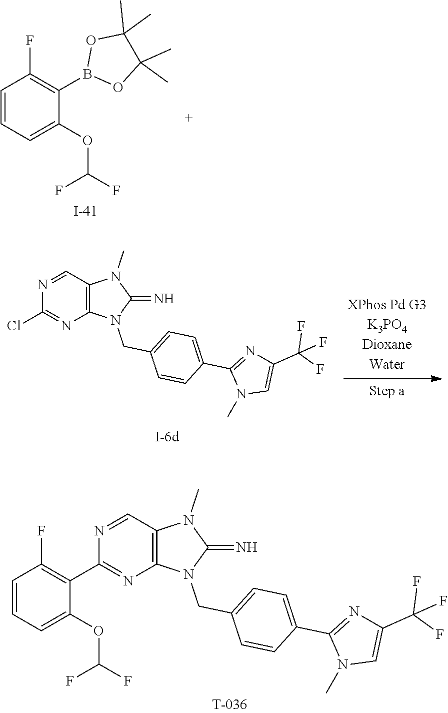

### Step a) 2-[2-(difluoromethoxy)-6-fluoro-phenyl]-7-methyl-9-[[4-[I-methyl-4-(trifluoromethyl)imidazol-2-yl]phenyl]methyl]purin-8-imine

2-chloro-7-methyl-9-[[4-[1-methyl-4-(trifluoromethyl)imidazol-2-yl]phenyl]methyl]purin-8-imine (80 mg, 190 μmol), 2-[2-(difluoromethoxy)-6-fluoro-phenyl]-4,4,5,5-tetramethyl-1,3,2-dioxaborolane (109 mg, 379.32 μmol) and dioxane/water (5/1, 5 mL) were mixed. The obtained mixture was evacuated and then backfilled with argon. XPhos Pd G3 (11.2 mg, 13.3 μmol) was added. The resulting mixture was stirred at 90° C. for 12 hr. The reaction mixture was cooled, SiliaMetS® Dimercaptotriazine (100 mg) was added and the mixture was stirred for 3 hr. The mixture was diluted with MTBE (5 mL), filtered through a pad of SiO₂ and concentrated under reduced pressure. The residue was subjected to HPLC (0.5-6.5 min 45% water—ACN; flow: 30 mL/min, column: Waters SunFire C18, 100×19 mm, 5 μm) to afford 2-[2-(difluoromethoxy)-6-fluoro-phenyl]-7-methyl-9-[[4-[1-methyl-4-(trifluoromethyl)imidazol-2-yl]phenyl]methyl]purin-8-imine (T-036)(7.0 mg, 12.8 μmol, 6.74% yield) as yellow oil which solidified upon freeze-drying into light-yellow powder.

¹H NMR (600 MHz, DMSO-d₆) δ 3.32-3.40 (m, 3H), 3.72 (s, 3H), 5.03-5.21 (m, 2H), 6.50-6.62 (m, 1H), 7.01-7.27 (m, 3H), 7.48 (d, 2H), 7.50-7.56 (m, 1H), 7.63 (d, 2H), 7.89 (s, 1H), 8.19-8.27 (m, 1H).

LCMS (ESI): [M+H]⁺ m/z: calcd 548.18; found 548.2.

### Example T-063

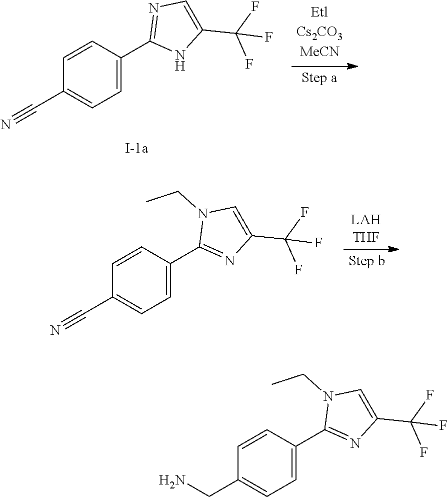 [SMILES: N#Cc1ccc(-c2ncc(C(F)(F)F)[nH]2)cc1 ; CCn1cc(C(F)(F)F)nc1-c1ccc(C#N)cc1 ; CCn1cc(C(F)(F)F)nc1-c1ccc(CN)cc1]

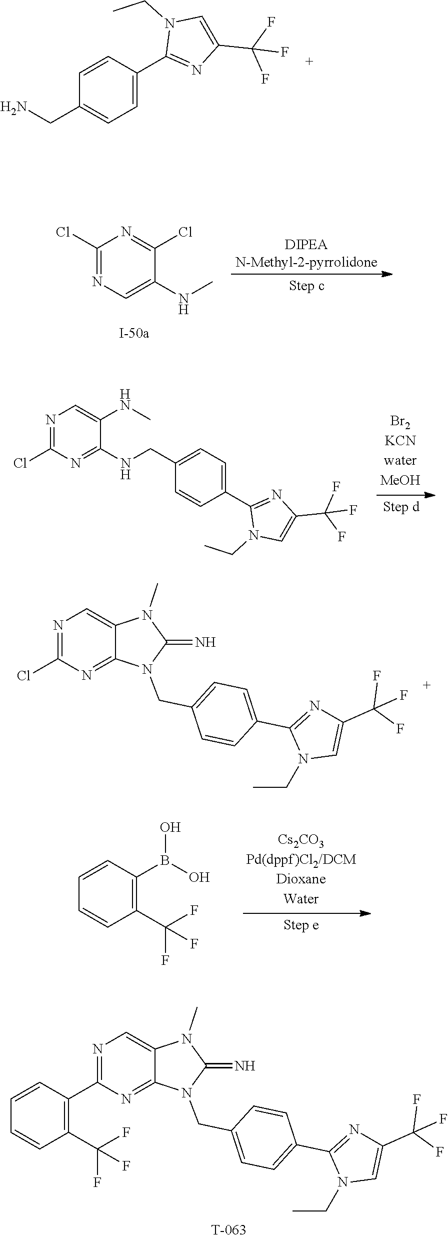

### Step a) 4-(I-ethyl-4-(trifluoromethyl)-1H-imidazol-2-yl)benzonitrile

Cesium carbonate (11.0 g, 33.7 mmol) and 4-(4-(trifluoromethyl)-1H-imidazol-2-yl)benzonitrile (4.0 g, 16.9 mmol) were mixed in acetonitrile (100 mL). Ethyl iodide (2.63 g, 16.7 mmol, 1.36 mL) in acetonitrile (10 ml) was added at ambient temperature to the above solution. The resulting mixture was stirred at ambient temperature for 12 hr. The reaction mixture was filtered and the filtrate was concentrated in vacuo. The residue was diluted with CH₂C₁₂ (50 mL), the solution was washed with water (20 mL) and brine (20 mL), dried over anhydrous sodium sulfate and concentrated in vacuo to afford 4-(1-ethyl-4-(trifluoromethyl)-1H-imidazol-2-yl)benzonitrile (4.0 g, 89% yield) as a yellow solid which was used in the next step without further purification.

¹H NMR (400 MHz, DMSO-d₆) δ 1.34 (t, 3H), 4.14 (q, 2H), 7.86 (d, 2H), 7.99 (d, 2H), 8.13 (s, 1H).

LCMS(ESI): [M+H]⁺ m/z: calcd 266.1; found 266.0.

### Step b) (4-(I-ethyl-4-(trifluoromethyl)-1H-imidazol-2-yl)phenyl)methanamine

To a suspension of lithium aluminum hydride (860 mg, 22.6 mmol) in tetrahydrofuran (100 mL) 4-[1-ethyl-4-(trifluoromethyl)imidazol-2-yl]benzonitrile (2.4 g, 9.05 mmol) in THE (50 mL) was added dropwise at vigorous stirring at 0° C. The resulting mixture was stirred at ambient temperature for 24 hr. The reaction mixture was quenched by dropwise addition of 0.9 mL of water diluted with 3.6 mL of THE followed by dropwise addition of 0.9 mL of 15% aqueous NaOH and 2.7 mL of water. The resulting solid was filtered out and rinsed with THF. The filtrate was concentrated under reduced pressure to give (4-(1-ethyl-4-(trifluoromethyl)-1H-imidazol-2-yl)phenyl)methanamine (2.5 g, 92.2% yield) as light-yellow viscous oil which was used in the next step without further purification.

¹H NMR (DMSO-d₆, 500 MHz) δ 1.32 (t, 3H), 3.79 (s, 2H), 4.09 (q, 2H), 7.48 (d, 2H), 7.55 (d, 2H), 8.00 (s, 1H).

LCMS(ESI): [M+H]⁺ m/z: calcd 270.14; found 270.2.

### Step c) 2-chloro-N4-(4-(I-ethyl-4-(trifluoromethyl)-1H-imidazol-2-yl)benzyl)-N₅-methylpyrimidine-4,5-diamine

To a stirred solution of [4-[1-ethyl-4-(trifluoromethyl)imidazol-2-yl]phenyl]methanamine (0.55 g, 2.04 mmol) in N-methyl-2-pyrrolidone (10 mL) N,N-diisopropylethylamine (792 mg, 6.13 mmol, 1.07 mL) and 2,4-dichloro-N-methyl-pyrimidin-5-amine (472.71 mg, 2.66 mmol) were added in an inert atmosphere. The resulting mixture was stirred at 90° C. for 12 hr. The reaction mixture was cooled, diluted with EtOAc (30 mL) and washed with H₂O (10 mL×3) and brine (10 mL). The organic layer was dried over anhydrous sodium sulfate and concentrated in vacuo to give 2-chloro-N4-(4-(1-ethyl-4-(trifluoromethyl)-1H-imidazol-2-yl)benzyl)-N₅-methylpyrimidine-4,5-diamine (0.70 g, 1.70 mmol, 83.4%) as light-yellow gum which was used in the next step without further purification.

LCMS(ESI): [M+H]⁺ m/z: calcd 411.15; found 411.2.

### Step d) 2-chloro-9-(4-(I-ethyl-4-(trifluoromethyl)-H-imidazol-2-yl)benzyl)-7-methyl-7H-purin-8(9H)-imine

A solution of potassium cyanide (555 mg, 8.52 mmol) in water (6 mL) was added dropwise at 0° C. to a stirred solution of bromine (1.36 g, 8.52 mmol) in water (2 mL). The resulting mixture was stirred at 0° C. for 15 min. To the above mixture a solution of 2-chloro-N₄-[[4-[1-ethyl-4-(trifluoromethyl)imidazol-2-yl]phenyl]methyl]-N5-methyl-pyrimidine-4,5-diamine (0.70 g, 1.70 mmol) in methanol (25 mL) was added at 0° C. The reaction mixture was stirred at 50° C. for 12 hr. The resulting mixture was concentrated in vacuo. The residue was diluted with MeOH and filtered. The filtrate was concentrated in vacuo to afford 2-chloro-9-(4-(1-ethyl-4-(trifluoromethyl)-1H-imidazol-2-yl)benzyl)-7-methyl-7H-purin-8(9H)-imine (0.80 g, crude, 71% by LCMS) as yellow gum which was used in the next steps without further purification. LCMS(ESI): [M+H]⁺ m/z: calcd 436.14; found 436.2.

### Step e) 9-(4-(I-ethyl-4-(trifluoromethyl)-1H-imidazol-2-yl)benzyl)-7-methyl-2-(2-(trifluoromethyl)phenyl)-7H-purin-8(9H)-imine

2-chloro-9-[[4-[1-ethyl-4-(trifluoromethyl)imidazol-2-yl]phenyl]methyl]-7-methyl-purin-8-imine (0.40 g, 918 μmol), (2-(trifluoromethyl)phenyl)boronic acid (349 mg, 1.84 mmol) and cesium carbonate (1.20 g, 3.67 mmol) were mixed in water (4 mL) and dioxane (20 mL). The resulting mixture was evacuated and then backfilled with argon. This operation was repeated three times, then bis(diphenylphosphino)ferrocene]dichloropalladium(II)-DCM (75 mg, 92 μmol) was added. The resulting mixture was stirred at 90° C. for 12 hr. The reaction mixture was cooled to room temperature and filtered. To the above filtrate SiliaMetS© Dimercaptotriazine (40 mg) was added. The obtained mixture was stirred at room temperature for 1 h, filtered and concentrated in vacuo. The residue was subjected to HPLC (0-5 min 40-90% water—ACN, +0.1% vol. of 25% aq. NH₃, 30 mL/min, column: YMC-Actus Triart C18, 100×20 mm, 5 μm) to afford 9-(4-(1-ethyl-4-(trifluoromethyl)-1H-imidazol-2-yl)benzyl)-7-methyl-2-(2-(trifluoromethyl)phenyl)-7H-purin-8(9H)-imine (32.0 mg, 58.7 μmol, 6.89% yield from 2-chloro-N₄-[[4-[1-ethyl-4-(trifluoromethyl)imidazol-2-yl]phenyl]methyl]-N5-methyl-pyrimidine-4,5-diamine) (T-063) as a brown solid.

¹H NMR (500 MHz, DMSO-d₆) δ 1.27 (t, 3H), 3.33-3.40 (m, 3H), 4.04 (q, 2H), 5.05-5.23 (m, 2H), 6.51-6.60 (m, 1H), 7.40-7.50 (m, 2H), 7.56 (d, 2H), 7.62 (t, 1H), 7.68-7.75 (m, 2H), 7.80 (d, 1H), 7.99 (s, 1H), 8.18-8.58 (m, 1H).

LCMS(ESI): [M+H]⁺ m/z: calcd 546.21; found 546.2.

### Example T-051

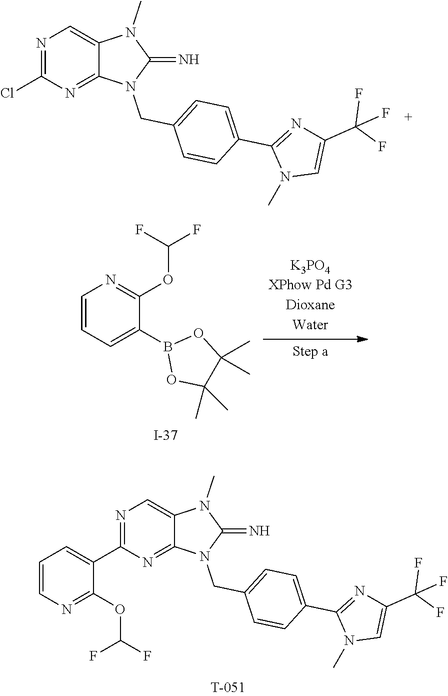

### Step a) 2-[2-(difluoromethoxy)-3-pyridyl]-9-[[4-[I-isopropyl-4-(trifluoromethyl)imidazol-2-yl]phenyl]methyl]-7-methyl-purin-8-imine

2-chloro-9-[[4-[1-isopropyl-4-(trifluoromethyl)imidazol-2-yl]phenyl]methyl]-7-methyl-purin-8-imine (0.21 g, 467 μmol), 2-(difluoromethoxy)-3-(4,4,5,5-tetramethyl-1,3,2-dioxaborolan-2-yl)pyridine (380 mg, 1.40 mmol) and potassium phosphate tribasic anhydrous (297 mg, 1.40 mmol) were dissolved in dioxane (7 mL) and water (0.2 mL). The resulting mixture was evacuated and then backfilled with argon. XPhos Pd G3 (10 mg, 23 μmol) was added to the above mixture. The resulting mixture was stirred at 95° C. for 16 hr. The reaction mixture was cooled, diluted with water (10 mL) and extracted with EtOAc (10 mL×2). The combined organic layers were washed with water (10 mL) and brine (10 mL), then dried over anhydrous sodium sulfate and concentrated under reduced pressure. The residue was subjected to HPLC (2-10 min 0-80% water—methanol, +0.1% vol. of 25% aq. NH₃, 30 mL/min, column: Waters SunFire C18, 100×19 mm, 5 μm) to afford 2-[2-(difluoromethoxy)-3-pyridyl]-9-[[4-[1-isopropyl-4-(trifluoromethyl)imidazol-2-yl]phenyl]methyl]-7-methyl-purin-8-imine (T-051)(14.0 mg, 5.4% yield) as a yellow solid.

¹H NMR (500 MHz, DMSO-d₆) δ 1.36 (d, 6H), 3.33-3.39 (m, 3H), 4.28-4.46 (m, 1H), 5.06-5.24 (m, 2H), 6.52-6.65 (m, 1H), 7.38 (dd, 1H), 7.47-7.53 (m, 2H), 7.57-7.61 (m, 2H), 7.77 (t, 1H, CHF₂), 8.14 (s, 1H), 8.20-8.34 (m, 3H).

LCMS(ESI): [M+H]⁺ m/z: calcd 559.22; found 559.4.

### Example T-095

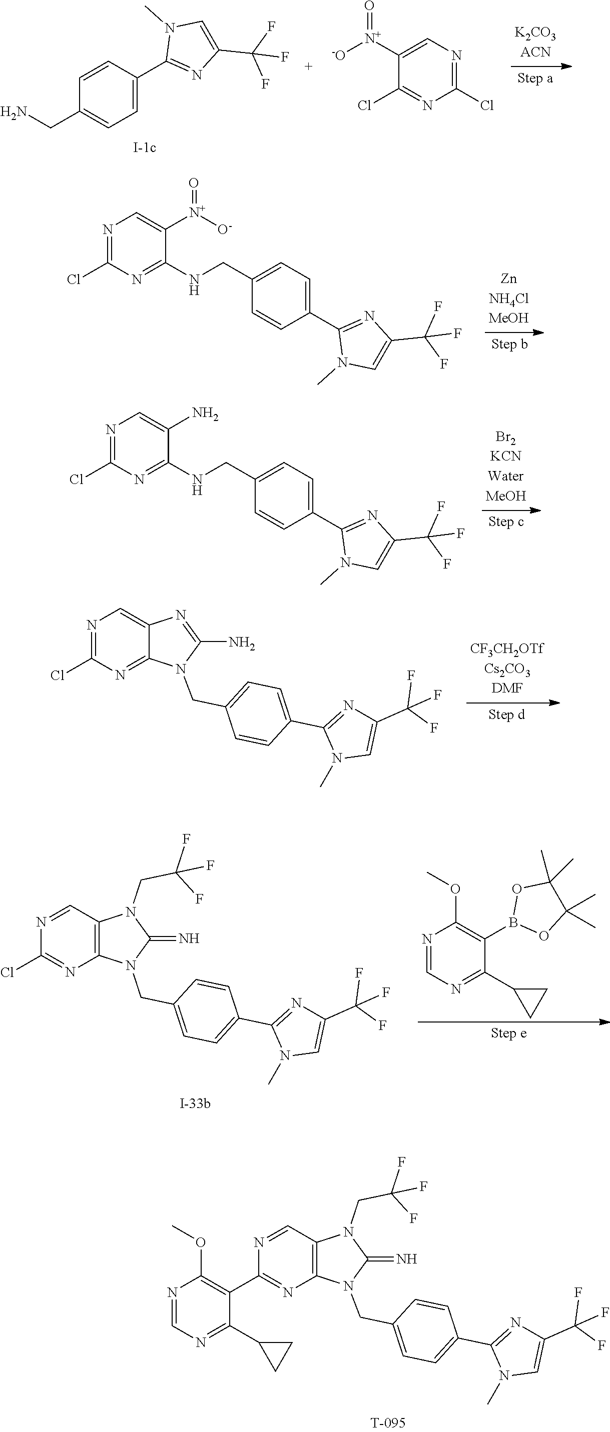

### Step a) 2-chloro-N-[[4-[I-methyl-4-(trifluoromethyl)imidazol-2-yl]phenyl]methyl]-5-nitro-pyrimidin-4-amine

To a solution of 2,4-dichloro-5-nitro-pyrimidine (1.12 g, 5.76 mmol) in ACN (20 mL) potassium carbonate (1.14 g, 8.23 mmol) was added and the reaction mixture was stirred for 5 min at room temperature. The mixture was cooled to −15° C. and [4-[1-methyl-4-(trifluoromethyl)imidazol-2-yl]phenyl]methanamine (1.40 g, 5.49 mmol) was added. The resulting mixture was stirred at room temperature for 18 hr. The reaction mixture was filtered. The filtrate was concentrated in vacuo. The residue was poured into H₂O (7.00 mL) and extracted with DCM (2×30. mL). The combined organic layers were washed with brine (10 mL), dried over anhydrous sodium sulfate and concentrated in vacuo to afford 2-chloro-N-[[4-[1-methyl-4-(trifluoromethyl)imidazol-2-yl]phenyl]methyl]-5-nitro-pyrimidin-4-amine (1.70 g, 75% yield) as solid which was used in the next step without further purification.

¹H NMR (500 MHz, CDCl₃) δ 3.76 (s, 3H), 4.90 (d, 2H), 7.26 (s, 1H), 7.46 (d, 2H), 7.65 (d, 2H), 9.07 (s, 1H).

LCMS(ESI): [M+H]⁺ m/z: calcd 413.07; found 413.1.

### Step b) 2-chloro-N₄-[[4-[1-methyl-4-(trifluoromethyl)imidazol-2-yl]phenyl]methyl]pyrimidine-4,5-diamine

To a solution of 2-chloro-N-[[4-[1-methyl-4-(trifluoromethyl)imidazol-2-yl]phenyl]methyl]-5-nitro-pyrimidin-4-amine (1.70 g, 4.12 mmol) and ammonium chloride (2.20 g, 41.2 mmol) in MeOH (60 mL), was added zinc dust (1.35 g, 20.6 mmol. The resulting mixture was stirred at room temperature for 24 hr. The reaction mixture was filtered. The filtrate was concentrated in vacuo. The residue was poured into water (10 mL) and extracted with EtOAc (3×30 mL). The combined organic layers were dried over anhydrous sodium sulfate and concentrated in vacuo to afford 2-chloro-N4-[[4-[1-methyl-4-(trifluoromethyl)imidazol-2-yl]phenyl]methyl]pyrimidine-4,5-diamine (1.20 g, 3.14 mmol, 76% yield) which was used in the next step without further purification.

¹H NMR (500 MHz, DMSO-d₆) δ 3.53 (br, 2H), 3.71 (s, 3H), 4.62 (d, 2H), 5.91 (br, 1H), 7.27-7.33 (m, 3H), 7.39-7.45 (m, 3H).

LCMS(ESI): [M+H]⁺ m/z: calcd 383.11; found 383.2.

### Step c) 2-chloro-9-[[4-[I-methyl-4-(trifluoromethyl)imidazol-2-yl]phenyl]methyl]purin-8-amine

Potassium cyanide (851 mg, 13.1 mmol) in water (25 mL) was added to a solution of bromine (2.09 g, 13.1 mmol, 1.40 mL) in MeOH (50 mL) at 0° C. The reaction mixture was stirred until decolorization occurred, then a solution of 2-chloro-N₄-[[4-[1-methyl-4-(trifluoromethyl)imidazol-2-yl]phenyl]methyl]pyrimidine-4,5-diamine (500 mg, 1.31 mmol) in MeOH (3 mL) was added. The resulting mixture was stirred at room temperature for 36 hr. The reaction mixture was concentrated in vacuo. The residue was poured into saturated aqueous NaHCO₃ (10 mL) and extracted with DCM (60 mL). The organic layer was dried over anhydrous sodium sulfate and concentrated in vacuo to afford 2-chloro-9-[[4-[1-methyl-4-(trifluoromethyl)imidazol-2-yl]phenyl]methyl]purin-8-amine (450 mg, 84% yield) as a solid which was used in the next step without further purification.

¹H NMR (400 MHz, CDCl₃) δ 3.73 (s, 3H), 4.61 (br, 1H), 5.13 (br, 1H), 5.32 (s, 2H), 7.22-7.33 (m, 3H), 7.57-7.64 (m, 2H), 8.45 (s, 1H).

LCMS(ESI): [M+H]⁺ m/z: calcd 408.1; found 408.2.

### Step d) 2-chloro-9-[[4-[I-methyl-4-(trifluoromethyl)imidazol-2-yl]phenyl]methyl]-7-(2,2,2-trifluoroethyl)purin-8-imine

To a solution of 2-chloro-9-[[4-[1-methyl-4-(trifluoromethyl)imidazol-2-yl]phenyl]methyl]purin-8-amine (150 mg, 368 μmol) in DMF (5 mL) was added cesium carbonate (179 mg, 552 μmol). The reaction mixture was stirred at 25° C. for 30 min, then 2,2,2-trifluoroethyl trifluoromethanesulfonate (94 mg, 405 μmol, 58 μL) was added. The resulting mixture was stirred at 65° C. for 15 hr. The reaction mixture was poured into H₂O (10.0 mL) and extracted with EtOAc (30 mL). The organic layer was dried over anhydrous sodium sulfate and concentrated in vacuo to afford 2-chloro-9-[[4-[1-methyl-4-(trifluoromethyl)imidazol-2-yl]phenyl]methyl]-7-(2,2,2-trifluoroethyl)purin-8-imine (200 mg, crude) as a brown solid.

LCMS(ESI): [M+H]⁺ m/z: calcd 490.11; found 490.1.

### Step e) 2-(4-cyclopropyl-6-methoxy-pyrimidin-5-yl)-9-[[4-[I-methyl-4-(trifluoromethyl)imidazol-2-yl]phenyl]methyl]-7-(2,2,2-trifluoroethyl)purin-8-imine

A mixture of water (0.25 mL) and dioxane (4 mL) was evacuated and then backfilled with argon three times, then 2-chloro-9-[[4-[1-methyl-4-(trifluoromethyl)imidazol-2-yl]phenyl]methyl]-7-(2,2,2-trifluoroethyl)purin-8-imine (100 mg, 204 μmol), 4-cyclopropyl-6-methoxy-5-(4,4,5,5-tetramethyl-1,3,2-dioxaborolan-2-yl)pyrimidine (169 mg, 612 μmol), potassium phosphate tribasic anhydrous (108 mg, 510 μmol), XPhos Pd G3 (13 mg, 15.3 μmol) and XPhos (7.3 mg, 15.3 μmol) were added under an inert atmosphere at room temperature. The reaction mixture was stirred at 80° C. for 15 hr. The reaction mixture was cooled to room temperature and concentrated in vacuo. The residue was diluted with H₂O (10 mL) and extracted with EtOAc (3×15 mL). The combined organic layers were dried over anhydrous sodium sulfate and concentrated in vacuo to give a crude product. The crude product was dissolved in methanol (5 mL) and treated with palladium scavenger SiliaMetS® Dimercaptotriazine (100 mg) at room temperature for 13 hr. The mixture was filtered and concentrated in vacuo to give a crude product which was triturated with hot diethyl ether and subjected to HPLC (gradient elution: 20-40% water—ACN, +0.1% vol. of 25% aq. NH₃, 30 mL/min, column: YMC-Actus Triart C18, 100×20 mm, 5 μm) to afford 2-(4-cyclopropyl-6-methoxy-pyrimidin-5-yl)-9-[[4-[1-methyl-4-(trifluoromethyl)imidazol-2-yl]phenyl]methyl]-7-(2,2,2-trifluoroethyl)purin-8-imine (T-095)(5.10 mg, 8.45 μmol, 4.14% yield) as an off-white powder.

¹H NMR (600 MHz, DMSO-d₆) δ 0.80-0.84 (m, 2H), 0.96-1.00 (m, 2H), 1.65-1.69 (m, 1H), 3.73 (s, 3H), 3.82 (s, 3H), 4.78-5.00 (m, 2H), 5.09-5.23 (m, 2H), 7.05-7.11 (m, 1H), 7.48 (d, 2H), 7.63-7.69 (m, 2H), 7.90 (s, 1H), 8.30-8.36 (m, 1H), 8.62 (s, 1H).

LCMS(ESI): [M+H]⁺ m/z: calcd 604.22; found 604.2.

### Example T-120

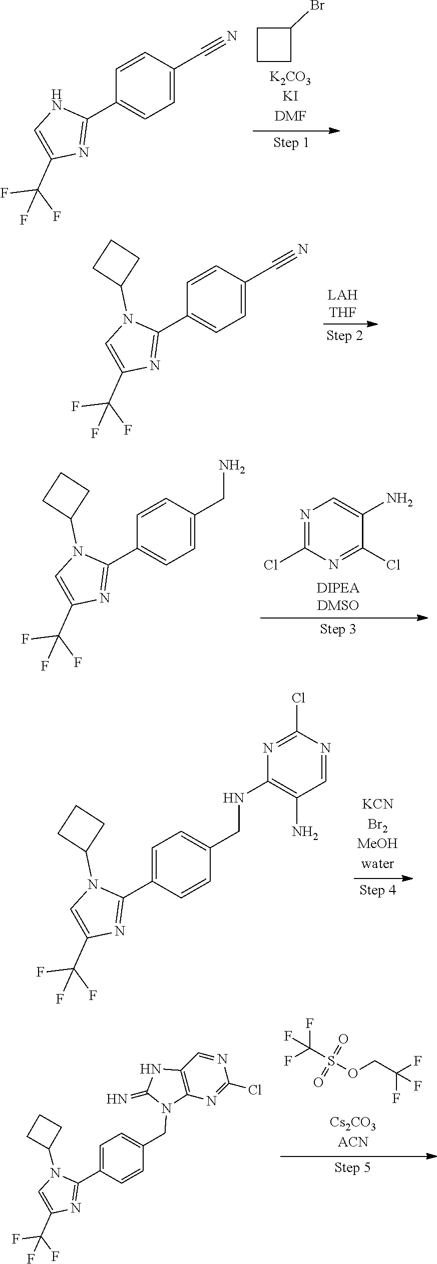

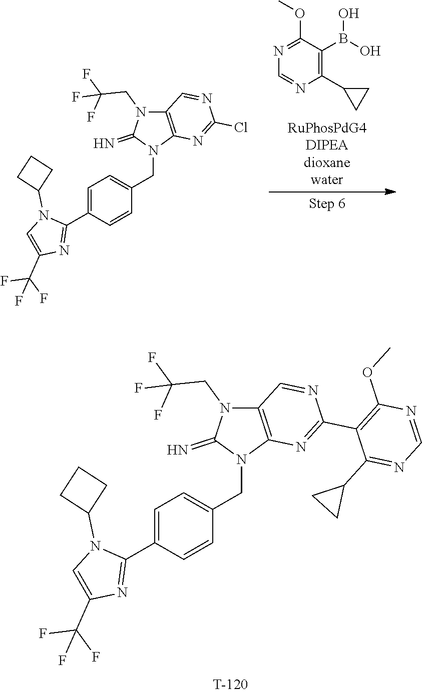 [SMILES: N=c1n(CC(F)(F)F)c2cnc(Cl)nc2n1Cc1ccc(-c2nc(C(F)(F)F)cn2C2CCC2)cc1 ; COc1ncnc(C2CC2)c1B(O)O ; COc1ncnc(C2CC2)c1-c1ncc2c(n1)n(Cc1ccc(-c3nc(C(F)(F)F)cn3C3CCC3)cc1)c(=N)n2CC(F)(F)F]

### Step 1: The synthesis of 4-[I-cyclobutyl-4-(trifluoromethyl)imidazol-2-yl]benzonitrile

Potassium carbonate anhydrous (909 mg, 6.58 mmol), potassium iodide (72 mg, 439 μmol) and cyclobutyl bromide (1.18 g, 8.77 mmol) were added to a solution of 4-[4-(trifluoromethyl)-1H-imidazol-2-yl]benzonitrile (1.04 g, 4.38 mmol) in DMF (5.0 mL). The reaction mixture was stirred at 90° C. for 72 hr. The reaction mixture was cooled to room temperature, diluted with water (100 mL) and extracted with EtOAc (100 mL). The organic layer was dried over anhydrous sodium sulfate and filtered. The filtrate was concentrated under reduced pressure. The residue was subjected to HPLC (gradient elution: 50-70% water—ACN, +0.1% vol. of 25% aq. NH₃, flow: 30 mL/min, column: YMC Triart C18 100×20 mm, 5 μm) to afford 4-[1-cyclobutyl-4-(trifluoromethyl)imidazol-2-yl]benzonitrile (312 mg, 1.07 mmol, 24% yield) as a yellow solid.

¹H NMR (500 MHz, CDCl₃) δ 1.79-2.00 (m, 2H), 2.32-2.43 (m, 2H), 2.48-2.56 (m, 2H), 4.70-4.79 (m, 1H), 7.60 (s, 1H), 7.69 (d, 2H), 7.78 (d, 2H).

LCMS(ESI): [M+H]⁺ m/z: calcd 292.13; found 292.0.

### Step 2: The synthesis of [4-[I-cyclobutyl-4-(trifluoromethyl)imidazol-2-yl]phenyl]methanamine

LAH (62 mg, 1.82 mmol) was added to vigorously stirred solution of 4-[1-cyclobutyl-4-(trifluoromethyl)imidazol-2-yl]benzonitrile (312 mg, 1.07 mmol) in THE (10 mL) at 0° C. The reaction mixture was allowed to warm and stirred at room temperature for 2 hr. The reaction mixture was cooled to 0° C. and quenched by addition of water (500 μL). The solids were filtered out. The filtrate was concentrated under reduced pressure to afford [4-[1-cyclobutyl-4-(trifluoromethyl)imidazol-2-yl]phenyl]methanamine (302 mg, 1.02 mmol, 95.5% yield) as a yellow oil which was used in the next steps without further purification.

LCMS(ESI): [M+H]⁺ m/z: calcd 296.17; found 296.2.

### Step 3: The synthesis of 2-chloro-N₄-[[4-[1-cyclobutyl-4-(trifluoromethyl)imidazol-2-yl]phenyl]methyl]pyrimidine-4,5-diamine

[4-[1-cyclobutyl-4-(trifluoromethyl)imidazol-2-yl]phenyl]methanamine (302 mg, 1.02 mmol), 2,4-dichloropyrimidin-5-amine (252 mg, 1.53 mmol) and DIPEA (264 mg, 2.05 mmol, 356 μL) were mixed in DMSO (1.3 mL). The reaction mixture was stirred at 90° C. for 14 hr. The reaction mixture was diluted with EtOAc (40 mL) and washed with brine (60 mL). The organic layer was dried over anhydrous sodium sulfate and concentrated under reduced pressure to afford 2-chloro-N4-[[4-[1-cyclobutyl-4-(trifluoromethyl)imidazol-2-yl]phenyl]methyl]pyrimidine-4,5-diamine (543 mg, crude) as a brown oil which was used in the next steps without further purification.

LCMS(ESI): [M+H]⁺ m/z: calcd 423.16; found 423.0.

### Step 4: The synthesis of 2-chloro-9-[[4-[1-cyclobutyl-4-(trifluoromethyl)imidazol-2-yl]phenyl]methyl]-7H-purin-8-imine

A solution of potassium cyanide (836 mg, 12.8 mmol) in water (3.0 mL) was added to a vigorously stirred solution of molecular bromine (2.05 g, 12.8 mmol) in MeOH (25 mL) at room temperature. The resulting mixture was stirred at room temperature for 15 min. A solution of 2-chloro-N₄-[[4-[1-cyclobutyl-4-(trifluoromethyl)imidazol-2-yl]phenyl]methyl]pyrimidine-4,5-diamine (543 mg, 1.28 mmol) in MeOH (2.0 mL) was added to the mixture. The reaction mixture was stirred at room temperature for 16 hr. The reaction mixture was concentrated under reduced pressure. The residue was diluted with EtOAc (80 mL) and washed with a solution of aqueous potassium carbonate (50 mL, 10%). The organic layer was dried over anhydrous sodium sulfate and filtered. The filtrate was concentrated under reduced pressure. The residue was subjected to HPLC (gradient elution: 40-65% water—ACN, flow 30 ml/min; column Chromatorex 18 SMB100-5T 100×19 mm, 5 m) to afford 2-chloro-9-[[4-[1-cyclobutyl-4-(trifluoromethyl)imidazol-2-yl]phenyl]methyl]-7H-purin-8-imine (147 mg, 328 μmol, 25.6% yield) as a yellow solid.

¹H NMR (500 MHz, DMSO-d₆) δ 1.61-1.76 (m, 2H), 2.30-2.42 (m, 4H), 4.68-4.76 (m, 1H), 5.35 (s, 2H), 7.34 (d, 2H), 7.47-7.56 (m, 3H), 8.26 (s, 1H), 8.31 (s, 1H).

LCMS(ESI): [M+H]⁺ m/z: calcd 448.15; found 448.0.

### Step 5: The synthesis of 2-chloro-9-[[4-[1-cyclobutyl-4-(trifluoromethyl)imidazol-2-yl]phenyl]methyl]-7-(2,2,2-trifluoroethyl)purin-8-imine

2,2,2-trifluoroethyl trifluoromethanesulfonate (85 mg, 367 μmol, 53 μL) was added to a stirred mixture of 9-[[4-[1-cyclobutyl-4-(trifluoromethyl)imidazol-2-yl]phenyl]methyl]-2-(4-cyclopropyl-6-methoxy-pyrimidin-5-yl)-7H-purin-8-imine (147 mg, 262 μmol) and cesium carbonate (171 mg, 524 μmol) in ACN (10 mL). The reaction mixture was stirred at 60° C. for 16 hr. The reaction mixture was cooled to room temperature and concentrated under reduced pressure. The residue was diluted with water (30 mL) and extracted with EtOAc (40 mL). The organic layer was washed with brine (30 mL), dried over anhydrous sodium sulfate and concentrated under reduced pressure to afford 2-chloro-9-[[4-[1-cyclobutyl-4-(trifluoromethyl)imidazol-2-yl]phenyl]methyl]-7-(2,2,2-trifluoroethyl)purin-8-imine (160 mg, crude) as a yellow solid which was used in the next steps without further purification.

LCMS(ESI): [M+H]⁺ m/z: calcd 530.13; found 530.2.

NOTE: the obtained crude also contains isomeric 2-chloro-9-[[4-[1-cyclobutyl-4-(trifluoromethyl)imidazol-2-yl]phenyl]methyl]-N-(2,2,2-trifluoroethyl)purin-8-amine.

### Step 6: The synthesis of9-[[4-[I-cyclobutyl-4-(trifluoromethyl)imidazol-2-yl]phenyl]methyl]-2-(4-cyclopropyl-6-methoxy-pyrimidin-5-yl)-7-(2,2,2-trifluoroethyl)purin-8-imine

(4-cyclopropyl-6-methoxy-pyrimidin-5-yl)boronic acid (104 mg, 536 μmol), DIPEA (139 mg, 1.07 mmol, 187 μL) and RuPhos Pd G4 (15.2 mg, 17.9 μmol) were added to a solution of 2-chloro-9-[[4-[1-cyclobutyl-4-(trifluoromethyl)imidazol-2-yl]phenyl]methyl]-7H-purin-8-imine (160 mg, 357 μmol) in a degassed mixture of dioxane (4 mL) and water (1 mL) under argon atmosphere. The reaction mixture was stirred at 95° C. for 16 hr. The reaction mixture was cooled to room temperature and concentrated under reduced pressure. The residue was diluted with EtOAc (20 mL) and washed with brine (10 mL). The organic layer was dried over anhydrous sodium sulfate and filtered. The filtrate was concentrated under reduced pressure. The residue was subjected to HPLC (gradient elution: 35-70% water+FA−ACN+FA, flow: 30 ml/min, column: Chromatorex 18 SMB100-5T 100×19 mm 5 m), then repurified by HPLC (gradient elution: 40-75% water−ACN+0.1% vol. of 25% aq. NH₃, flow: 30 mL/min, column: YMC Triart C18 100×20 mm, 5 μm) to afford 9-[[4-[1-cyclobutyl-4-(trifluoromethyl)imidazol-2-yl]phenyl]methyl]-2-(4-cyclopropyl-6-methoxy-pyrimidin-5-yl)-7-(2,2,2-trifluoroethyl)purin-8-imine (20.6 mg, 32.0 μmol, 8.96% yield) as a yellow solid.

¹H NMR (600 MHz, DMSO-d₆) δ 0.79-0.84 (m, 2H), 0.97-1.02 (m, 2H), 1.61-1.76 (m, 3H), 2.29-2.41 (m, 4H), 3.82 (s, 3H), 4.66-4.74 (m, 1H), 4.77-5.02 (m, 2H), 5.07-5.26 (m, 2H), 7.04-7.13 (m, 1H), 7.46-7.53 (m, 4H), 8.26 (s, 1H), 8.30-8.37 (m, 1H), 8.62 (s, 1H).

LCMS(ESI): [M+H]⁺ m/z: calcd 644.27; found 644.0.

### Example T-127

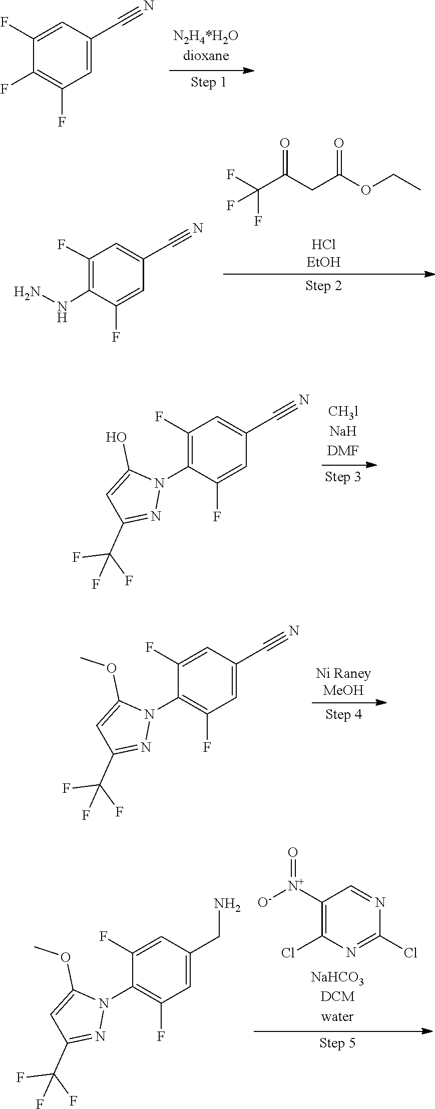

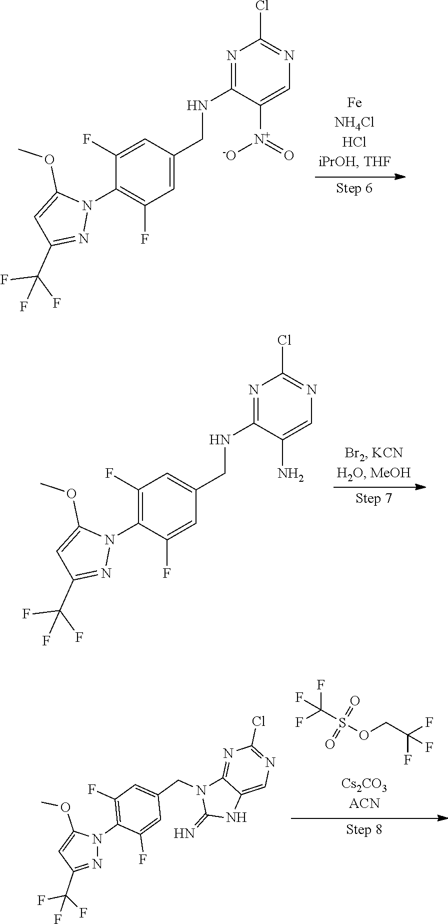

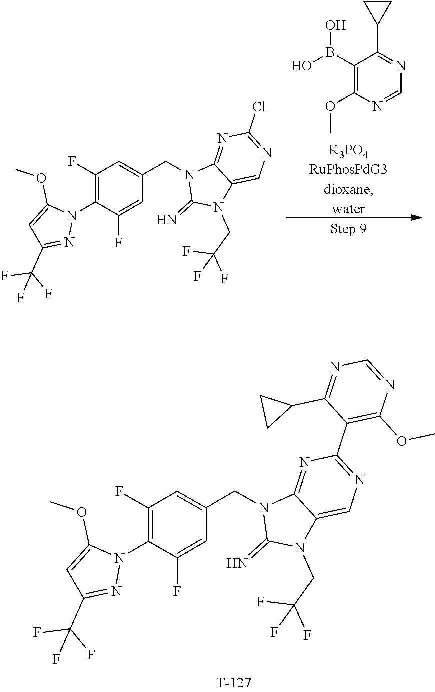 [SMILES: COc1cc(C(F)(F)F)nn1-c1c(F)cc(Cn2c(=N)n(CC(F)(F)F)c3cnc(Cl)nc32)cc1F ; COc1ncnc(C2CC2)c1B(O)O ; "K3PO4RuPhosPdG3dioxane,water" ; COc1ncnc(C2CC2)c1-c1ncc2c(n1)n(Cc1cc(F)c(-n3nc(C(F)(F)F)cc3OC)c(F)c1)c(=N)n2CC(F)(F)F]

### Step 1: The Synthesis of 3,5-difluoro-4-hydrazino-benzonitrile

A mixture of 3,4,5-trifluorobenzonitrile (10.4 g, 66.2 mmol) and aqueous hydrazine monohydrate (4.52 mL, 80% wt.) in Dioxane (150 mL) was stirred at 50° C. for 64 hr. The reaction mixture was cooled to room temperature and concentrated under reduced pressure. The residue was triturated with cold MTBE (50 mL) and filtered off to afford 3,5-difluoro-4-hydrazino-benzonitrile (7.50 g, 44.3 mmol, 67.0% yield) as a light-yellow solid which was used in the next steps without further purification.

¹H NMR (500 MHz, DMSO-d6) δ 4.52 (s, 2H), 7.29 (s, 1H), 7.40-7.45 (m, 2H).

GCMS: [M+H]+ m/z: calcd 169.05; found 169.0.

### Step 2: The synthesis of 3,5-difluoro-4-[5-hydroxy-3-(trifluoromethyl)pyrazol-1-yl]benzonitrile

A solution of aqueous hydrochloric acid (559 μL, 36% wt.) was added to a solution of 3,5-difluoro-4-hydrazino-benzonitrile (5.20 g, 30.8 mmol) and ethyl 4,4,4-trifluoro-3-oxo-butanoate (5.94 g, 32.3 mmol, 4.72 mL) in EtOH (125 mL). The reaction mixture was stirred at 78° C. for 16 hr. The reaction mixture was cooled to room temperature and concentrated under reduced pressure to afford 3,5-difluoro-4-[5-hydroxy-3-(trifluoromethyl)pyrazol-1-yl]benzonitrile (8.80 g, 30.4 mmol, 98.0% yield) as a brown solid which was used in the next steps without further purification.

LCMS(ESI): [M+H]+ m/z: calcd 290.04; found 290.0.

### Step 3: The synthesis of 3,5-difluoro-4-[5-methoxy-3-(trifluoromethyl)pyrazol-1-yl]benzonitrile

3,5-difluoro-4-[5-hydroxy-3-(trifluoromethyl)pyrazol-1-yl]benzonitrile (8.80 g, 22.8 mmol) and iodomethane (7.13 g, 50.2 mmol, 3.13 mL) were sequentially added to a stirred suspension of sodium hydride (1.40 g, 36.5 mmol, 60% dispersion in mineral oil) in DMF (150 mL). The reaction mixture was stirred at room temperature for 24 hr. The reaction mixture was poured into cold water (400 mL) and extracted with MTBE (4×75 mL). The combined organic layers were washed with water (3×50 mL) and brine (2×40 mL), dried over anhydrous sodium sulfate and concentrated under reduced pressure. The residue was purified by flash column chromatography (Hexanes—EtOAc, gradient, from pure hexanes to 25% EtOAc) to afford 3,5-difluoro-4-[5-methoxy-3-(trifluoromethyl)pyrazol-1-yl]benzonitrile (4.61 g, 15.2 mmol, 66.6% yield) as a light-yellow solid.

¹H NMR (500 MHz, DMSO-d6) δ 3.99 (s, 3H), 6.57 (s, 1H), 8.16 (d, 2H).

LCMS(ESI): [M+H]+ m/z: calcd 304.05; found 304.2.

### Step 4: The synthesis of [3,5-difluoro-4-[5-methoxy-3-(trifluoromethyl)pyrazol-1-yl]phenyl]methanamine

A solution of 3,5-difluoro-4-[5-methoxy-3-(trifluoromethyl)pyrazol-1-yl]benzonitrile (1.20 g, 3.96 mmol) in MeOH (100 mL) was subjected to hydrogenation for 12 hr. at 100 bar using Ni Raney (3.96 mmol) as a catalyst. The reaction mixture was filtered through the thin pad of silica. The filtrate was concentrated under reduced pressure to afford [3,5-difluoro-4-[5-methoxy-3-(trifluoromethyl)pyrazol-1-yl]phenyl]methanamine (1.00 g, 3.26 mmol, 82.0% yield) as a yellow gum which was used for the next step without further purification.

¹H NMR (500 MHz, DMSO-d6) δ 3.83 (s, 2H), 3.95 (s, 3H), 6.48 (s, 1H), 7.38 (d, 2H).

LCMS(ESI): [M+H]+ m/z: calcd 308.08; found 308.2.

### Step 5: The synthesis of 2-chloro-N-[[3,5-difluoro-4-[5-methoxy-3-(trifluoromethyl)pyrazol-1-yl]phenyl]methyl]-5-nitro-pyrimidin-4-amine

A solution of 2,4-dichloro-5-nitro-pyrimidine (590 mg, 3.04 mmol) in dichloromethane (10 mL) was added dropwise to a stirred mixture of [3,5-difluoro-4-[5-methoxy-3-(trifluoromethyl)pyrazol-1-yl]phenyl]methanamine (1.10 g, 3.04 mmol) in dichloromethane (75 mL) and sodium bicarbonate (511 mg, 6.09 mmol) in water (15 mL) at 0° C. The reaction mixture was stirred at 0° C. for 30 min and then at room temperature for 15 hr. The reaction mixture was diluted with water (20 mL) and extracted with DCM (3×40 mL). The combined organic layers were washed with brine (2×25 mL), dried over anhydrous sodium sulfate and concentrated under reduced pressure to afford 2-chloro-N-[[3,5-difluoro-4-[5-methoxy-3-(trifluoromethyl)pyrazol-1-yl]phenyl]methyl]-5-nitro-pyrimidin-4-amine (1.40 g, 3.01 mmol, 99.3% yield) as a yellow solid which was used in the next steps without further purification.

¹H NMR (500 MHz, DMSO-d6) δ 3.96 (s, 3H), 4.83 (d, 2H), 6.49 (s, 1H), 7.42 (d, 2H), 9.08 (s, 1H), 9.67 (t, 1H).

LCMS(ESI): [M+H]+ m/z: calcd 465.05; found 465.0.

### Step 6: The synthesis of 2-chloro-N4-[[3,5-difluoro-4-[5-methoxy-3-(trifluoromethyl)pyrazol-1-yl]phenyl]methyl]pyrimidine-4,5-diamine

Iron powder (1.74 g, 31.2 mmol) was added portionwise to a stirred mixture of 2-chloro-N-[[3,5-difluoro-4-[5-methoxy-3-(trifluoromethyl)pyrazol-1-yl]phenyl]methyl]-5-nitro-pyrimidin-4-amine (1.45 g, 3.12 mmol), ammonium chloride (1.17 g, 21.8 mmol) and aqueous hydrochloric acid (1.44 mL, 36% wt) in THF (25 mL) and IPA (25 mL). The reaction mixture was stirred at 45° C. for 18 hr. The reaction mixture was cooled to room temperature and filtered. The filter cake was washed with IPA (20 mL). The combined filtrate was concentrated under reduced pressure. The residue was diluted with an aqueous solution of potassium carbonate (20 mL, 10% wt.) and extracted with EtOAc (4×50 mL). The combined organic layers were washed with brine (30 mL), dried over anhydrous sodium sulfate and filtered through the thin pad of silica. The filtrate was concentrated under reduced pressure to afford 2-chloro-N4-[[3,5-difluoro-4-[5-methoxy-3-(trifluoromethyl)pyrazol-1-yl]phenyl]methyl]pyrimidine-4,5-diamine (1.15 g, 2.65 mmol, 84.6% yield) as a yellow solid which was used in the next steps without further purification.

LCMS(ESI): [M+H]+ m/z: calcd 435.08; found 435.0.

### Step 7: The synthesis of 2-chloro-9-[[3,5-difluoro-4-[5-methoxy-3-(trifluoromethyl)pyrazol-1-yl]phenyl]methyl]-7H-purin-8-imine

A solution of potassium cyanide (1.27 g, 19.5 mmol) in water (8.0 mL) was added dropwise to a solution of bromine (3.12 g, 19.5 mmol, 2.0 mL) in MeOH (15 mL) at 0° C. The reaction mixture was stirred at 0° C. for 15 min. A solution of 2-chloro-N4-[[3,5-difluoro-4-[5-methoxy-3-(trifluoromethyl)pyrazol-1-yl]phenyl]methyl]pyrimidine-4,5-diamine (1.14 g, 2.62 mmol) in MeOH (15 mL) was added to the mixture. The resulting mixture was stirred at 35° C. for 48 hr. The reaction mixture was cooled to room temperature and poured into a solution of aqueous K2CO3 (100 mL, 10% wt.). The resulting mixture was concentrated under reduced pressure. The residue was diluted with water (30 mL) and extracted with EtOAc (5×50 mL). The combined organic layers were dried over anhydrous sodium sulfate and filtered through the thin pad of silica gel. The filtrate was dried over anhydrous sodium sulfate and concentrated under reduced pressure to afford 2-chloro-9-[[3,5-difluoro-4-[5-methoxy-3-(trifluoromethyl)pyrazol-1-yl]phenyl]methyl]-7H-purin-8-imine (1.10 g, 2.39 mmol, 90.9% yield) as a yellow solid which was used in the next steps without further purification.

LCMS(ESI): [M+H]+ m/z: calcd 460.07; found 460.0.

### Step 8: The synthesis of 2-chloro-9-[[3,5-difluoro-4-[5-methoxy-3-(trifluoromethyl)pyrazol-1-yl]phenyl]methyl]-7-(2,2,2-trifluoroethyl)purin-8-imine

Cesium carbonate (1.19 g, 3.65 mmol) was added to a solution of 2-chloro-9-[[3,5-difluoro-4-[5-methoxy-3-(trifluoromethyl)pyrazol-1-yl]phenyl]methyl]-7H-purin-8-imine (1.05 g, 1.83 mmol) in ACN (125 mL). The reaction mixture was stirred at room temperature for 15 min. 2,2,2-trifluoroethyl trifluoromethanesulfonate (636 mg, 2.74 mmol, 395 μL) was added to the reaction mixture. The resulting mixture was stirred at 70° C. for 16 hr. The reaction mixture was cooled to room temperature, then solids were filtered out. The filtrate was concentrated under reduced pressure. The residue was diluted with EtOAc (200 mL) and washed with water (75 mL). The aqueous layer was extracted with EtOAc (3×40 mL). The combined organic layers were washed with brine (2×30 ml), dried over anhydrous sodium sulfate and concentrated under reduced pressure. The residue was subjected to HPLC (2-10 min, 40-95% ACN+FA (0.1% vol.); flow: 30 mL/min, column: Waters SunFire C18, 100×19 mm, 5 μm) to afford 2-chloro-9-[[3,5-difluoro-4-[5-methoxy-3-(trifluoromethyl)pyrazol-1-yl]phenyl]methyl]-7-(2,2,2-trifluoroethyl)purin-8-imine (148 mg, 273 μmol, 15.0% yield) as a brown gum.

LCMS(ESI): [M+H]+ m/z: calcd 542.09; found 542.2.

NOTE: 2-chloro-9-[[3,5-difluoro-4-[5-methoxy-3-(trifluoromethyl)pyrazol-1-yl]phenyl]methyl]-N-(2,2,2-trifluoroethyl)purin-8-amine and 2-chloro-9-[[3,5-difluoro-4-[5-methoxy-3-(trifluoromethyl)pyrazol-1-yl]phenyl]methyl]-N,7-bis(2,2,2-trifluoroethyl)purin-8-imine are also formed in this reaction.

### Step 9: The synthesis of 2-(4-cyclopropyl-6-methoxy-pyrimidin-5-yl)-9-[[3,5-difluoro-4-[5-methoxy-3-(trifluoromethyl)pyrazol-1-yl]phenyl]methyl]-7-(2,2,2-trifluoroethyl)purin-8-imine

2-chloro-9-[[3,5-difluoro-4-[5-methoxy-3-(trifluoromethyl)pyrazol-1-yl]phenyl]methyl]-7-(2,2,2-trifluoroethyl)purin-8-imine (80.0 mg, 148 μmol), (4-cyclopropyl-6-methoxy-pyrimidin-5-yl)boronic acid (57.3 mg, 295 μmol) and potassium phosphate (69.0 mg, 325 μmol) were mixed in a degassed mixture of dioxane (8.0 mL) and water (1.0 mL). The reaction mixture was degassed. RuPhos Pd G3 (25.1 mg, 29.5 μmol) was added to the reaction mixture. The reaction mixture was stirred at 95° C. for 24 hr. The reaction mixture was cooled to room temperature and concentrated under reduced pressure. The residue was diluted with ACN (8.0 mL) and filtered. Metal scavengers SiliaMetS® Dimercaptotriazine (50 mg) was added to the filtrate. The resulting suspension was stirred at room temperature for 10 hr., then solids were filtered out. The filtrate was subjected to HPLC (2-10 min, 0-50% ACN+FA (0.1% vol.); flow: 30 mL/min, column: Waters SunFire C18, 100×19 mm, 5 μm) to afford 2-(4-cyclopropyl-6-methoxy-pyrimidin-5-yl)-9-[[3,5-difluoro-4-[5-methoxy-3-(trifluoromethyl)pyrazol-1-yl]phenyl]methyl]-7-(2,2,2-trifluoroethyl)purin-8-imine (46.0 mg, 70.2 μmol, 47.5% yield) as a beige solid.

¹H NMR (600 MHz, DMSO-d6) δ 0.78-0.83 (m, 2H), 0.97-1.02 (m, 2H), 1.67-1.73 (m, 1H), 3.80 (s, 3H), 3.93 (s, 3H), 4.71-5.02 (m, 2H), 5.10-5.28 (m, 2H), 6.48 (s, 1H), 7.12 (br., 1H), 7.39 (br., 2H), 8.34 (br., 1H), 8.62 (s, 1H).

LCMS(ESI): [M+H]+ m/z: calcd 656.2; found 656.2.

### Example T-109

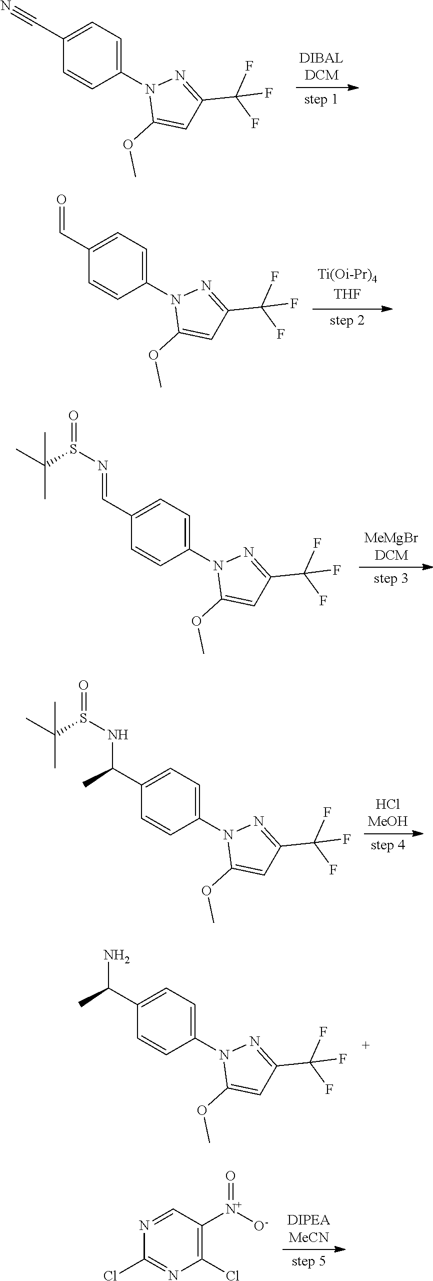

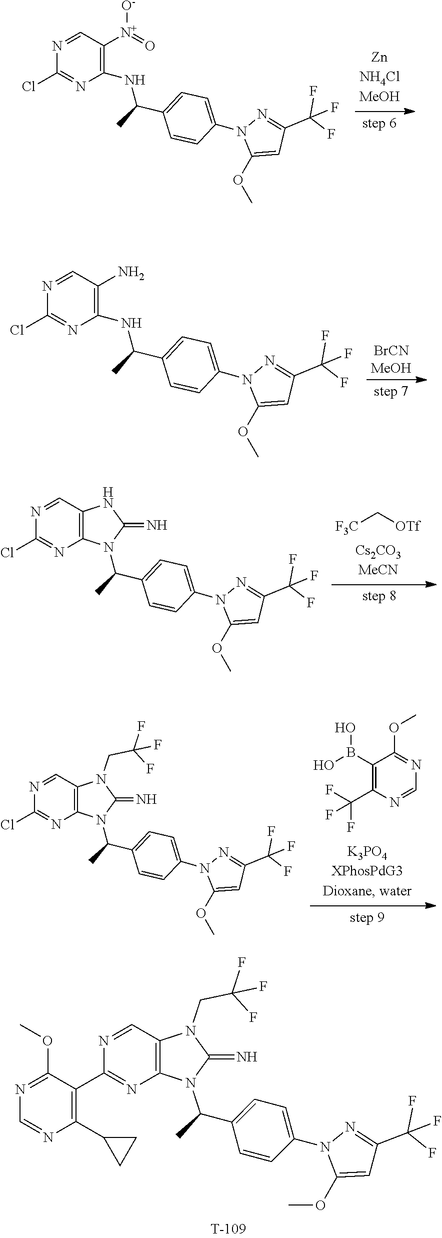

### Step 1: Synthesis of 4-[5-methoxy-3-(trifluoromethyl)pyrazol-1-yl]benzaldehyde

Diisobutylaluminum hydride (1.49 g, 10.5 mmol, 10.5 mL, 1M solution in hexane) was added dropwise to a solution of 4-[5-methoxy-3-(trifluoromethyl)pyrazol-1-yl]benzonitrile (2.00 g, 7.48 mmol) in methylene chloride (100 mL) at −60° C. under argon atmosphere. The resulting mixture was stirred for 3 hr, during which time it was allowed to warm to −30° C. A mixture of i-PrOH (10 mL) and water (10 mL) was added dropwise to the reaction mixture at −30° C. The resulting mixture was stirred at 0° C. for 30 minutes. The reaction was filtered and the filtrate was washed with brine (20 mL), dried over anhydrous sodium sulfate and concentrated under reduced pressure to afford 4-[5-methoxy-3-(trifluoromethyl)pyrazol-1-yl]benzaldehyde (1.60 g, 5.92 mmol, 79.1% yield) as a red oil which was used in the next steps without further purification.

¹H NMR (400 MHz, CDCl3) δ 4.03 (s, 3H), 5.97 (s, 1H), 7.96 (s, 4H), 10.02 (s, 1H).

GCMS: [M−H]+ m/z: calcd 271.08; found 271.0.

### Step 2: Synthesis of (S)—N-(4-(5-methoxy-3-(trifluoromethyl)-1H-pyrazol-1-yl)benzylidene)-2-methylpropane-2-sulfinamide

Titanium (IV) isopropoxide (2.21 g, 7.77 mmol) was added dropwise to a stirred solution of 4-[5-methoxy-3-(trifluoromethyl)pyrazol-1-yl]benzaldehyde (1.40 g, 5.18 mmol) and 2-methylpropane-2-sulfinamide (691 mg, 5.70 mmol) in THE (10 mL). The resulting mixture was stirred at room temperature for 24 hr. The reaction mixture was diluted with aqueous solution of NaHCO₃ (10 mL, 5% wt.) and EtOAc (20 mL) and stirred for 15 minutes. The reaction was filtered and the filtrate was washed with brine (2×10 mL) and concentrated under reduced pressure to afford (S)—N-(4-(5-methoxy-3-(trifluoromethyl)-1H-pyrazol-1-yl)benzylidene)-2-methylpropane-2-sulfinamide (1.60 g, 4.29 mmol, 82.7% yield) as a red oil which was used in the next steps without further purification.

LCMS(ESI): [M+H]+ m/z: calcd 374.12; found 374.0.

### Step 3: The synthesis of (S)-2-methyl-N-[(1R)-]-[4-[5-methoxy-3-(trifluoromethyl)pyrazol-1-yl]phenyl]ethyl]propane-2-sulfinamide

Methylmagnesium bromide (747 mg, 6.43 mmol, 2.14 mL, 3M in ether) was added dropwise to a solution of (S)—N-(4-(5-methoxy-3-(trifluoromethyl)-1H-pyrazol-1-yl)benzylidene)-2-methylpropane-2-sulfinamide (1.60 g, 4.29 mmol) in methylene chloride (30 mL) at −60° C. under argon atmosphere. The resulting mixture was stirred for 3 hr, during which time it was allowed to warm to room temperature. The reaction mixture was cooled 0° C. and quenched by dropwise addition of saturated aqueous solution of NH4Cl (20 mL). The resulting mixture was stirred for 15 min. The organic layer was separated, washed with brine (10 mL) and concentrated under reduced pressure. The residue was subjected to flash-column chromatography (SiO2, gradient elution: DCM—acetonitrile) to afford (S)-2-methyl-N-[(1R)-1-[4-[5-methoxy-3-(trifluoromethyl)pyrazol-1-yl]phenyl]ethyl]propane-2-sulfinamide (0.45 g, 1.16 mmol, 27.0% yield) as a yellow oil.

¹H NMR (400 MHz, DMSO-d6) δ 1.11 (s, 9H), 1.46 (d, 3H), 3.99 (s, 3H), 4.42-4.50 (m, 1H), 5.40-5.45 (m, 1H), 6.46 (s, 1H), 7.49 (d, 2H), 7.57 (d, 2H).

LCMS(ESI): [M+H]+ m/z: calcd 390.15; found 390.0.

### Step 4: Synthesis of (1R)-1-[4-[5-methoxy-3-(trifluoromethyl)pyrazol-1-yl]phenyl]ethanamine

HCl in dioxane (3 mL, 4M) was added dropwise to a solution of (S)-2-methyl-N-[(1R)-1-[4-[5-methoxy-3-(trifluoromethyl)pyrazol-1-yl]phenyl]ethyl]propane-2-sulfinamide (0.42 g, 1.08 mmol) in MeOH (4 mL). The reaction mixture was stirred at room temperature for 24 hr. The reaction mixture was concentrated under reduced pressure. The residue was partitioned between DCM (10 mL) and saturated aqueous NH4OH (5 mL). The water layer was extracted with DCM (2×5 mL). The combined organic layers were washed with brine (5 mL) and concentrated under reduced pressure to afford (1R)-1-[4-[5-methoxy-3-(trifluoromethyl)pyrazol-1-yl]phenyl]ethanamine (0.30 g, 1.05 mmol, 97.5% yield) as a yellow solid which was used in the next steps without further purification.

LCMS(ESI): [M+H]+ m/z: calcd 286.14; found 286.0.

### Step 5: Synthesis of 2-chloro-5-nitro-N-[(1R)-1-[4-[5-methoxy-3-(trifluoromethyl)pyrazol-1-yl]phenyl]ethyl]pyrimidin-4-amine

2,4-Dichloro-5-nitro-pyrimidine (204 mg, 1.05 mmol) was added in one portion to a stirred solution of (1R)-1-[4-[5-methoxy-3-(trifluoromethyl)pyrazol-1-yl]phenyl]ethanamine (0.30 g, 1.05 mmol) and DIPEA (150 mg, 1.16 mmol) in dioxane (5 mL) at room temperature. The resulting mixture was stirred at room temperature for 12 hr. The reaction mixture was diluted with water (10 mL). The resulting mixture was extracted with ethyl acetate (2×10 mL). The combined organic layers were washed with brine (10 mL) and concentrated under reduced pressure to afford 2-chloro-5-nitro-N-[(1R)-1-[4-[5-methoxy-3-(trifluoromethyl)pyrazol-1-yl]phenyl]ethyl]pyrimidin-4-amine (0.40 g, 903 μmol, 85.9% yield) as a red solid which was used in the next steps without further purification.

LCMS(ESI): [M+H]+ m/z: calcd 443.09; found 443.0.

### Step 6: Synthesis of 2-chloro-N4-[(1R)-1-[4-[5-methoxy-3-(trifluoromethyl)pyrazol-1-yl]phenyl]ethyl]pyrimidine-4,5-diamine

To a mixture of 2-chloro-5-nitro-N-[(1R)-1-[4-[5-methoxy-3-(trifluoromethyl)pyrazol-1-yl]phenyl]ethyl]pyrimidin-4-amine (0.40 g, 903 μmol) and ammonium chloride (725 mg, 13.6 mmol) in MeOH (10 mL) was added zinc powder (354 mg, 5.42 mmol) portionwise at 0° C. The resulting mixture was stirred at room temperature for 14 hr. The reaction mixture was filtered and the filtrate was concentrated under reduced pressure. The residue was diluted with water (10 mL) and extracted with EtOAc (2×20 mL). The combined organic layers were washed with water (10 mL) and brine (10 mL) and concentrated under reduced pressure to afford 2-chloro-N4-[(1R)-1-[4-[5-methoxy-3-(trifluoromethyl)pyrazol-1-yl]phenyl]ethyl]pyrimidine-4,5-diamine (0.32 g, 775 mol, 85.8% yield) as a yellow solid which was used in the next steps without further purification.

LCMS(ESI): [M+H]+ m/z: calcd 413.13; found 413.0.

### Step 7: Synthesis of 2-chloro-9-[(1R)-1-[4-[5-methoxy-3-(trifluoromethyl)pyrazol-1-yl]phenyl]ethyl]-7H-purin-8-imine

BrCN (346 mg, 3.27 mmol) was added to a stirred solution of 2-chloro-N4-[(1R)-1-[4-[5-methoxy-3-(trifluoromethyl)pyrazol-1-yl]phenyl]ethyl]pyrimidine-4,5-diamine (0.27 g, 654 mol) in methanol (10 mL) in one portion at room temperature. The resulting mixture was stirred at room temperature for 12 hr. The reaction mixture was concentrated under reduced pressure. The residue was partitioned between EtOAc (20 mL) and saturated aqueous NaHCO3 solution (10 mL). The organic layer was separated, washed with brine (100 mL) and concentrated under reduced pressure to afford 2-chloro-9-[(1R)-1-[4-[5-methoxy-3-(trifluoromethyl)pyrazol-1-yl]phenyl]ethyl]-7H-purin-8-imine (0.24 g, 548 μmol, 83.8% yield) as a brown solid which was used in the next steps without further purification.

LCMS(ESI): [M+H]+ m/z: calcd 438.12; found 438.0.

### Step 8: Synthesis of 2-chloro-9-[(1R)-1-[4-[5-methoxy-3-(trifluoromethyl)pyrazol-1-yl]phenyl]ethyl]-7-(2,2,2-trifluoroethyl)purin-8-imine

To a stirred mixture of 2-chloro-9-[(1R)-1-[4-[5-methoxy-3-(trifluoromethyl)pyrazol-1-yl]phenyl]ethyl]-7H-purin-8-imine (0.24 g, 548 μmol) and cesium carbonate (268 mg, 822 μmol) in ACN (5 mL) was added 2,2,2-trifluoroethyl trifluoromethanesulfonate (152 mg, 658 μmol) dropwise. The resulting mixture was stirred at 50 C for 16 hr. The reaction mixture was cooled to room temperature and diluted with water (5 mL). The resulting mixture was extracted with ethyl acetate (2×10 mL). The combined organic layers were washed with water (2×10 mL) and brine (10 mL). The organic layer was concentrated under reduced pressure to afford 2-chloro-9-[(1R)-1-[4-[5-methoxy-3-(trifluoromethyl)pyrazol-1-yl]phenyl]ethyl]-7-(2,2,2-trifluoroethyl)purin-8-imine (0.30 g, crude) as a brown oil which was used in the next steps without further purification.

LCMS(ESI): [M+H]+ m/z: calcd 520.13; found 520.0.

### Step 9: Synthesis of 2-(4-cyclopropyl-6-methoxy-pyrimidin-5-yl)-9-[(1R)-1-[4-[5-methoxy-3-(trifluoromethyl)pyrazol-1-yl]phenyl]ethyl]-7-(2,2,2-trifluoroethyl)purin-8-imine

2-Chloro-9-[(1R)-1-[4-[5-methoxy-3-(trifluoromethyl)pyrazol-1-yl]phenyl]ethyl]-7-(2,2,2-trifluoroethyl)purin-8-imine (0.30 g, crude), (4-cyclopropyl-6-methoxy-pyrimidin-5-yl)boronic acid (202 mg, 1.04 mmol), potassium phosphate tribasic anhydrous (306 mg, 1.44 mmol) and XPhos Pd G3 (24.4 mg, 28.8 μmol) were mixed in a degassed mixture of dioxane (3 mL) and water (0.3 mL). The reaction mixture was stirred at 80° C. for 12 hr. The reaction mixture was cooled to room temperature, diluted with EtOAc (10 mL) and washed with water (5 mL) and brine (5 mL). To the obtained organic phase was added SiliaMetS® Dimercaptotriazine (20 mg), and the mixture was stirred for 30 min. The mixture was filtered and the filtrate was concentrated under reduced pressure. The residue was subjected to HPLC (gradient elutione: 0.5-6.5 min, 30-55% water+FA (0.1% vol.)—ACN+FA (0.1% vol.); flow: 30 mL/min, column: Waters SunFire C18, 100×19 mm, 5 μm) to afford 2-(4-cyclopropyl-6-methoxy-pyrimidin-5-yl)-9-[(1R)-1-[4-[5-methoxy-3-(trifluoromethyl)pyrazol-1-yl]phenyl]ethyl]-7-(2,2,2-trifluoroethyl)purin-8-imine (7.0 mg, 11 μmol, 2.0% yield from 2-chloro-9-[(1R)-1-[4-[5-methoxy-3-(trifluoromethyl)pyrazol-1-yl]phenyl]ethyl]-7H-purin-8-imine) as a light-yellow solid.

¹H NMR (600 MHz, DMSO-d6) δ 0.75-0.85 (m, 2H), 0.94-1.03 (m, 2H), 1.64-1.70 (m, 1H), 1.95 (d, 3H), 3.82 (s, 3H), 3.97 (s, 3H), 4.76-5.03 (m, 2H), 5.83-5.93 (m, 1H), 6.44 (s, 1H) 6.91-7.17 (m, 1H), 7.52-7.62 (m, 4H), 8.29-8.37 (m, 1H), 8.62 (s, 1H).

LCMS(ESI): [M+H]+ m/z: calcd 634.24; found 634.2.

### Example T-111

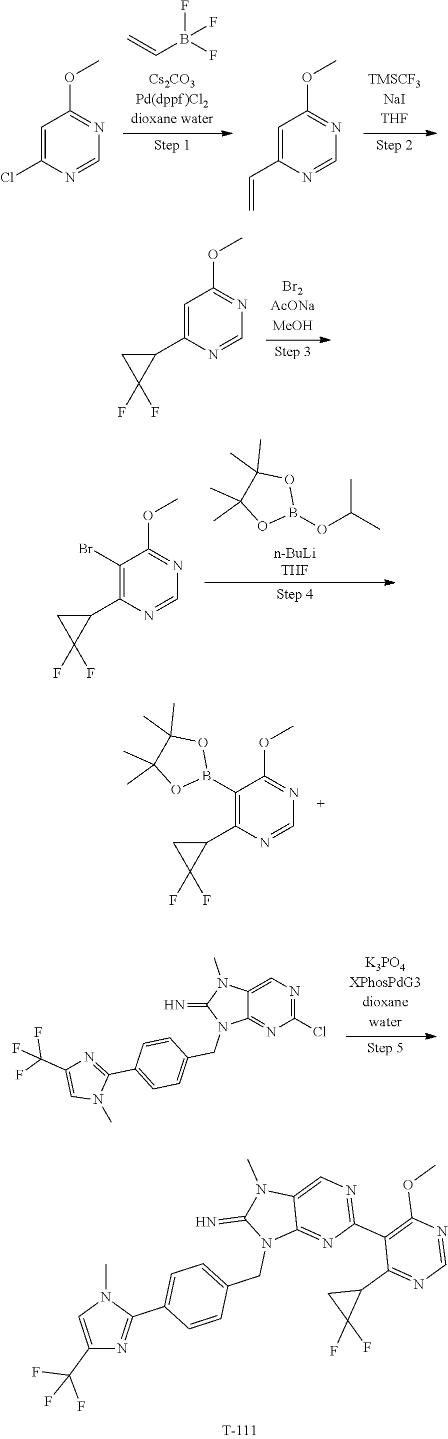

### Step 1: The synthesis of 4-methoxy-6-vinyl-pyrimidine

4-chloro-6-methoxy-pyrimidine (14.0 g, 96.9 mmol), potassium trifluoro(vinyl)boranuide (26.0 g, 194 mmol), cesium carbonate (78.9 g, 242 mmol), and bis(diphenylphosphino)ferrocene]dichloropalladium(II)-DCM (100 mg, 1.94 mmol) were mixed in a degassed mixture of dioxane (400 mL) and water (50 mL) under argon atmosphere. The reaction mixture was stirred at 95° C. for 16 hr. The reaction mixture was cooled to room temperature and concentrated under reduced pressure. The residue was diluted with EtOAc (400 mL) and washed with water (200 mL). The organic layer was separated, washed with brine (100 mL), dried over anhydrous sodium sulfate and concentrated under reduced pressure. The residue was dissolved in MTBE (250 mL) and filtered through a pad of silica gel. The filtrate was concentrated under reduced pressure to afford 4-methoxy-6-vinyl-pyrimidine (8.00 g, 58.8 mmol, 60.7% yield) as a brown solid.

¹H NMR (400 MHz, CDCl3) δ 3.97 (s, 3H), 5.60 (d, 1H), 6.39 (d, 1H), 6.59-6.69 (m, 2H), 8.71 (s, 1H).

LCMS(ESI): [M+H]+ m/z: calcd 137.08; found 137.0.

### Step 2: The synthesis of 4-(2,2-difluorocyclopropyl)-6-methoxy-pyrimidine

Trimethyl(trifluoromethyl)silane (25.1 g, 177 mmol, 28.0 mL) was added to a solution of 4-methoxy-6-vinyl-pyrimidine (6.00 g, 44.1 mmol) and sodium iodide (3.30 g, 22.0 mmol) in THE (100 mL). The reaction mixture was stirred at 60° C. for 1 hr. Sodium iodide (550 mg) and trimethyl(trifluoromethyl)silane (25.1 g, 177 mmol, 28.0 mL) were added to the reaction mixture. The resulting mixture was stirred at 60° C. for 2 hr. The reaction mixture was cooled to room temperature. The solids were filtered out. The filtrate was concentrated under reduced pressure. The residue was diluted with EtOAc (50 mL) and washed with water (20 mL). The organic layer was washed with brine (3×5 mL), dried over anhydrous sodium sulfate and concentrated under reduced pressure to afford 4-(2,2-difluorocyclopropyl)-6-methoxy-pyrimidine (6.00 g, 32.2 mmol, 73.2% yield) as a brown oil which was used in the next steps without further purification.

¹H NMR (400 MHz, DMSO-d6) δ 1.99-2.10 (m, 1H), 2.27-2.40 (m, 1H), 3.06-3.18 (m, 1H), 3.91 (s, 3H), 7.03 (s, 1H), 8.71 (s, 1H).

### Step 3: The synthesis of 5-bromo-4-(2,2-difluorocyclopropyl)-6-methoxy-pyrimidine

Bromine (5.15 g, 32.2 mmol) was added dropwise to a solution of 4-(2,2-difluorocyclopropyl)-6-methoxy-pyrimidine (3.00 g, 16.1 mmol) and sodium bicarbonate (1.62 g, 19.3 mmol, 753 μL) in MeOH (80 mL) at room temperature. The reaction mixture was stirred at room temperature for 48 hr. The reaction mixture was cooled to room temperature. The residue was diluted with water (60 mL) and extracted with EtOAc (200 mL). The organic layer was separated, washed with a solution of aqueous Na2S2O3 (30 mL, 5% wt.), water (30 mL) and brine (10 mL), dried over anhydrous sodium sulfate and concentrated under reduced pressure.

The residue was subjected to flash column chromatography (SiO2, gradient chloroform—MTBE) to afford 5-bromo-4-(2,2-difluorocyclopropyl)-6-methoxy-pyrimidine (600 mg, 2.26 mmol, 14.1% yield) as a yellow oil.

¹H NMR (400 MHz, CDCl3) δ 1.82-1.93 (m, 1H), 2.45-2.55 (m, 1H), 3.17-3.27 (m, 1H), 4.09 (s, 3H), 8.59 (s, 1H).

### Step 4: The synthesis of 4-(2,2-difluorocyclopropyl)-6-methoxy-5-(4,4,5,5-tetramethyl-1,3,2-dioxaborolan-2-yl)pyrimidine

5-bromo-4-(2,2-difluorocyclopropyl)-6-methoxy-pyrimidine (1.00 g, 3.77 mmol) and 2-isopropoxy-4,4,5,5-tetramethyl-1,3,2-dioxaborolane (1.05 g, 5.66 mmol) were dissolved in THE (70 mL) under argon atmosphere. The solution was cooled to −78° C. n-Butyllithium (6.79 mmol, 2.72 mL, 2.5 M in hexane) was added dropwise to the solution. The reaction mixture was stirred at −70° C. for 3 hr. The reaction mixture was allowed to warm to room temperature, quenched by dropwise addition of a saturated aqueous solution of NH4Cl (30 mL) and extracted with EtOAc (40 mL). The organic layer was separated, washed with brine (2×30 mL), dried over anhydrous sodium sulfate and concentrated under reduced pressure to afford 4-(2,2-difluorocyclopropyl)-6-methoxy-5-(4,4,5,5-tetramethyl-1,3,2-dioxaborolan-2-yl)pyrimidine (1.10 g, 3.52 mmol, 93.2% yield) as a brown oil which was used in the next steps without further purification.

LCMS(ESI): [M+H]+ m/z: calcd 313.18; found 313.0.

### Step 5: The synthesis of 2-(4-(2,2-difluorocyclopropyl)-6-methoxypyrimidin-5-yl)-7-methyl-9-(4-(1-methyl-4-(trifluoromethyl)-1H-imidazol-2-yl)benzyl)-7H-purin-8(9H)-imine

4-(2,2-difluorocyclopropyl)-6-methoxy-5-(4,4,5,5-tetramethyl-1,3,2-dioxaborolan-2-yl)pyrimidine (300 mg, 961 μmol), 2-chloro-7-methyl-9-(4-(1-methyl-4-(trifluoromethyl)-1H-imidazol-2-yl)benzyl)-7H-purin-8(9H)-imine (135 mg, 320 μmol), potassium phosphate tribasic anhydrous (204 mg, 961 μmol) and XPhosPdG3 (27.3 mg, 32.2 μmol) were mixed in a degassed mixture of dioxane (6.0 mL) and water (1.0 mL) under argon atmosphere. The reaction mixture was stirred at 80° C. for 24 hr. The reaction mixture was cooled to room temperature, diluted with EtOAc (30 mL) and washed with water (15 mL). The organic layer was separated, washed with brine (20 mL), dried over anhydrous sodium sulfate and filtered. SiliaMetS® Dimercaptotriazine (150 mg) was added to the filtrate and the resulting mixture was stirred for 1 hr. The resulting mixture was filtered. The filtrate was concentrated under reduced pressure. The residue was subjected to HPLC (2-10 min., 27-50% ACN, +0.1% vol. of 25% aq. NH3, flow: 30 mL/min, column: SunFire 100×19 mm, 5 μm), then repurified by HPLC (2-10 min., 35-60% ACN, +0.1% vol. of 25% aq. NH3, flow: 30 mL/min, column: SunFire 100×19 mm, 5 μm) to afford 2-(4-(2,2-difluorocyclopropyl)-6-methoxypyrimidin-5-yl)-7-methyl-9-(4-(1-methyl-4-(trifluoromethyl)-1H-imidazol-2-yl)benzyl)-7H-purin-8(9H)-imine (6.00 mg, 10.5 μmol, 3.28% yield) as a white solid.

¹H NMR (600 MHz, DMSO-d6) δ 1.83-1.92 (m, 1H), 2.28-2.36 (m, 1H), 2.71-2.82 (m, 1H), 3.34-3.39 (m, 3H), 3.72 (s, 3H), 3.87 (s, 3H), 5.02-5.26 (m, 2H), 6.55-6.63 (m, 1H), 7.48 (d, 2H), 7.62-7.66 (m, 2H), 7.89 (s, 1H), 8.20-8.28 (m, 1H), 8.77 (s, 1H).

LCMS(ESI): [M+H]+ m/z: calcd 572.22; found 572.

### Example T-131

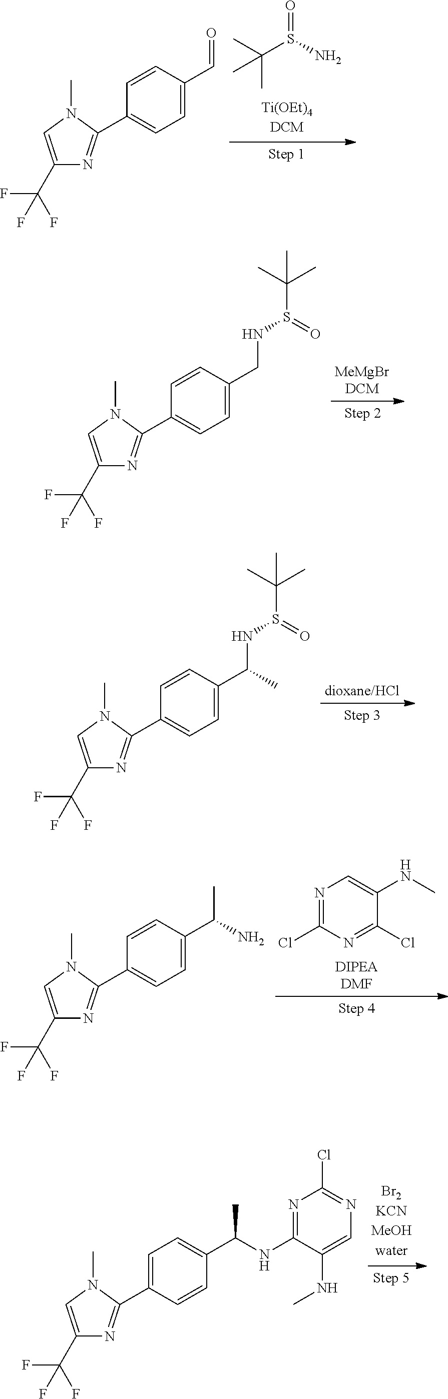

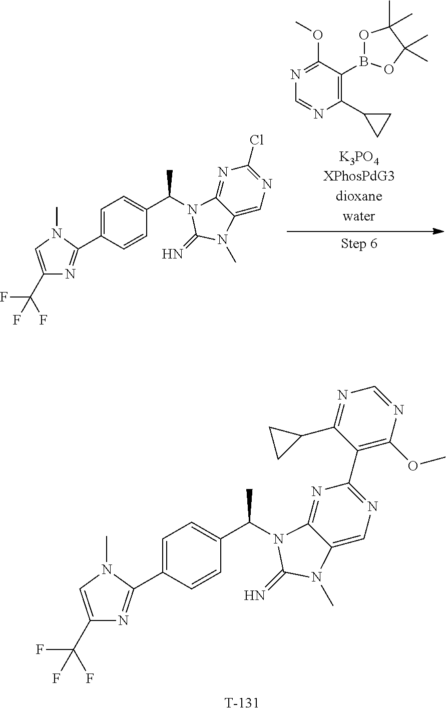 [SMILES: COc1ncnc(C2CC2)c1B1OC(C)(C)C(C)(C)O1 ; C[C@H](c1ccc(-c2nc(C(F)(F)F)cn2C)cc1)n1c(=N)n(C)c2cnc(Cl)nc21 ; COc1ncnc(C2CC2)c1-c1ncc2c(n1)n([C@H](C)c1ccc(-c3nc(C(F)(F)F)cn3C)cc1)c(=N)n2C]

### Step 1: The synthesis of 2-methyl-N-[[4-[I-methyl-4-(trifluoromethyl)imidazol-2-yl]phenyl]methylene]propane-2-sulfinamide as a yellow solid which was used in the next steps without further purification

Titanium (IV) ethoxide (2.56 g, 11.2 mmol, 2.35 mL) was added to a solution of 4-[1-methyl-4-(trifluoromethyl)imidazol-2-yl]benzaldehyde (570 mg, 2.24 mmol) in DCM (3.00 mL). The reaction mixture was stirred at room temperature for 20 min. (S)-2-methylpropane-2-sulfinamide (272 mg, 2.24 mmol) was added to the reaction mixture. The resulting mixture was stirred at room temperature for 18 hr. The reaction mixture was diluted with DCM (30 mL) and washed with a solution of aqueous NaHCO3 (20 mL), then solids were filtered out. The filtrate was extracted with DCM. The combined organic layers were dried over anhydrous sodium sulfate and concentrated under reduced pressure to afford 2-methyl-N-[[4-[1-methyl-4-(trifluoromethyl)imidazol-2-yl]phenyl]methylene]propane-2-sulfinamide (650 mg, 1.82 mmol, 81.11% yield) as a yellow solid which was used in the next steps without further purification.

LCMS(ESI): [M+H]+ m/z: calcd 411.16; found 411.2.

### Step 2: The synthesis of 2-methyl-N-[(1R)-1-[4-[1-methyl-4-(trifluoromethyl)imidazol-2-yl]phenyl]ethyl]propane-2-sulfinamide

Methylmagnesium bromide (1.08 g, 9.09 mmol, 1.05 mL) was added to a precooled to -30° C. solution of 2-methyl-N-[[4-[1-methyl-4-(trifluoromethyl)imidazol-2-yl]phenyl]methylene]propane-2-sulfinamide (650 mg, 1.82 mmol) in DCM (40 mL). The reaction mixture was stirred at −30° C. for 1 hr. The reaction mixture was allowed to warm up to room temperature, quenched by addition of acetone (1.0 mL) and washed with water (2×20 mL). The combined organic layers were dried over anhydrous sodium sulfate and concentrated under reduced pressure to afford 2-methyl-N-[(1R)-1-[4-[1-methyl-4-(trifluoromethyl)imidazol-2-yl]phenyl]ethyl]propane-2-sulfinamide (655 mg, 1.75 mmol, 96.4% yield) as a brown solid which was used in the next steps without further purification.

LCMS(ESI): [M+H]+ m/z: calcd 374.19; found 374.0.

### Step 3: The synthesis of (1R)-1-[4-[-methyl-4-(trifluoromethyl)imidazol-2-yl]phenyl]ethanamine

2-methyl-N-[(1R)-1-[4-[1-methyl-4-(trifluoromethyl)imidazol-2-yl]phenyl]ethyl]propane-2-sulfinamide (655 mg, 1.75 mmol) was suspended in a solution of hydrogen chloride in dioxane (8.00 mg, 219 mmol, 10.0 mL, 4.0 M). The reaction mixture was stirred at room temperature for 18 hr. The reaction mixture was concentrated under reduced pressure to afford (1R)-1-[4-[1-methyl-4-(trifluoromethyl)imidazol-2-yl]phenyl]ethanamine (610 mg, crude, HCl) as an off-white solid which was used in the next steps without further purification.

LCMS(ESI): [M+H]+ m/z: calcd 270.15; found 270.2.

### Step 4: The synthesis of 2-chloro-N5-methyl-N4-[(1S)-1-[4-[I-methyl-4-(trifluoromethyl)imidazol-2-yl]phenyl]ethyl]pyrimidine-4,5-diamine

2,4-dichloro-N-methyl-pyrimidin-5-amine (807 mg, 4.53 mmol) and DIPEA (878 mg, 6.80 mmol, 1.18 mL) were added to a solution of (1R)-1-[4-[1-methyl-4-(trifluoromethyl)imidazol-2-yl]phenyl]ethanamine (610 mg, 2.27 mmol, HCl) in DMF (10 mL).

The reaction mixture was stirred at 100° C. for 90 hr. The reaction mixture was cooled to room temperature, diluted with water (50 mL) and extracted with EtOAc (3×30 mL). The combined organic layers were concentrated under reduced pressure. The residue was subjected to HPLC (0.5-6.5 min., 50-80% water—MeOH, flow: 30 mL/min, column: XBridge C18 100×19 mm, 5 μm) to afford 2-chloro-N5-methyl-N4-[(1R)-1-[4-[1-methyl-4-(trifluoromethyl)imidazol-2-yl]phenyl]ethyl]pyrimidine-4,5-diamine (127 mg, 309 μmol, 13.7% yield) as a yellow solid.

LCMS(ESI): [M+H]+ m/z: calcd 411.16; found 411.4.

### Step 5: The synthesis of 2-chloro-7-methyl-9-[(1R)-1-[4-[1-methyl-4-(trifluoromethyl)imidazol-2-yl]phenyl]ethyl]purin-8-imine

A solution of potassium cyanide (297 mg, 4.56 mmol) in water (5.0 mL) was added dropwise to a solution of Br2 (729 mg, 4.56 mmol) in MeOH (20 mL) at 0° C. The resulting mixture was stirred at 0° C. for 15 min. A solution of 2-chloro-N5-methyl-N4-[(1R)-1-[4-[1-methyl-4-(trifluoromethyl)imidazol-2-yl]phenyl]ethyl]pyrimidine-4,5-diamine (125 mg, 304 mol) in MeOH (2.0 mL) was added to the mixture. The reaction mixture was stirred at room temperature for 18 hr. The reaction mixture was diluted with water (30 mL) and extracted with EtOAc (3×10 mL). The combined organic layers were dried over anhydrous sodium sulfate and concentrated under reduced pressure to afford 2-chloro-7-methyl-9-[(1R)-1-[4-[1-methyl-4-(trifluoromethyl)imidazol-2-yl]phenyl]ethyl]purin-8-imine (110 mg, 252 μmol, 83.0% yield) as a brown solid which was used in the next step without further purification.

LCMS(ESI): [M+H]+ m/z: calcd 436.15; found 436.2.

### Step 6: The synthesis of 2-(4-cyclopropyl-6-methoxy-pyrimidin-5-yl)-7-methyl-9-[(1S)-1-[4-[1-methyl-4-(trifluoromethyl)imidazol-2-yl]phenyl]ethyl]purin-8-imine

2-chloro-7-methyl-9-[(1R)-1-[4-[1-methyl-4-(trifluoromethyl)imidazol-2-yl]phenyl]ethyl]purin-8-imine (110 mg, 252 μmol) was dissolved in dioxane (3.0 mL) and water (200 μL). The resulting mixture was degassed twice. 4-cyclopropyl-6-methoxy-5-(4,4,5,5-tetramethyl-1,3,2-dioxaborolan-2-yl)pyrimidine (105 mg, 379 μmol), potassium phosphate (160 mg, 754 μmol) and XPhosPdG3 (20.0 mg, 23.6 μmol) were added to the mixture. The reaction mixture was stirred at 90° C. for 18 hr. The reaction mixture was cooled to room temperature, diluted with methanol (10 mL) and treated with SiliaMetS DMT. The resulting mixture was stirred at room temperature for 10 hr, then solids were filtered out. The filtrate was concentrated under reduced pressure. The residue was subjected to HPLC (0.5-6.5 min., 30-55% water—ACN, +0.1% vol. of 25% aq. NH3, flow: 30 mL/min, column: XBridge C18 100×19 mm, 5 μm) to afford 2-(4-cyclopropyl-6-methoxy-pyrimidin-5-yl)-7-methyl-9-[(1R)-1-[4-[1-methyl-4-(trifluoromethyl)imidazol-2-yl]phenyl]ethyl]purin-8-imine (9.40 mg, 17.1 μmol, 6.78% yield) as a yellow solid.

LCMS(ESI): [M+H]+ m/z: calcd 550.26; found 550.4.

### Example T-118

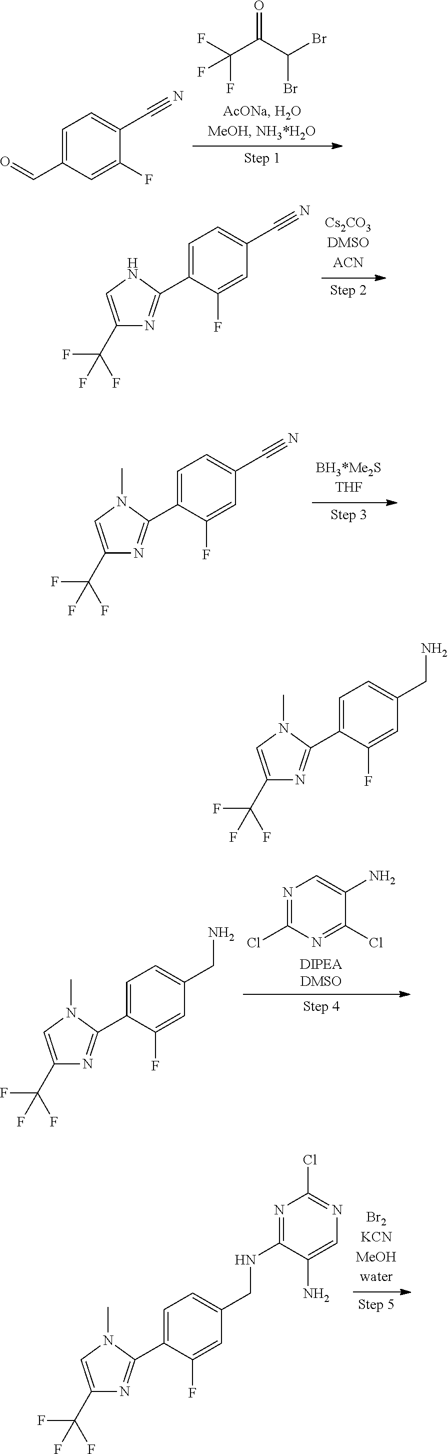

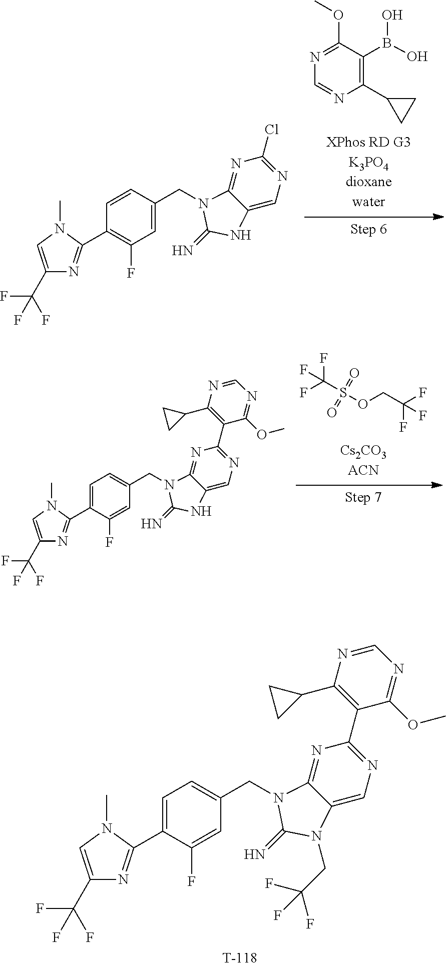 [SMILES: COc1ncnc(C2CC2)c1B(O)O ; Cn1cc(C(F)(F)F)nc1-c1ccc(Cn2c(=N)[nH]c3cnc(Cl)nc32)cc1F ; COc1ncnc(C2CC2)c1-c1ncc2[nH]c(=N)n(Cc3ccc(-c4nc(C(F)(F)F)cn4C)c(F)c3)c2n1 ; O=S(=O)(OCC(F)(F)F)C(F)(F)F ; COc1ncnc(C2CC2)c1-c1ncc2c(n1)n(Cc1ccc(-c3nc(C(F)(F)F)cn3C)c(F)c1)c(=N)n2CC(F)(F)F]

### Step 1: The synthesis of 3-fluoro-4-[4-(trifluoromethyl)-1H-imidazol-2-yl]benzonitrile

A solution of 3,3-dibromo-1, 1,1-trifluoro-propan-2-one (9.95 g, 36.9 mmol, 5.03 mL) and sodium acetate (6.05 g, 73.8 mmol) in water (20 mL) was stirred at 95° C. for 45 min. The reaction mixture was cooled to room temperature. To the reaction mixture was added a solution of 3-fluoro-4-formyl-benzonitrile (5.00 g, 33.5 mmol) followed by a solution of aqueous ammonium hydroxide (15 mL, 25% wt.) in MeOH (80 mL). The reaction mixture was stirred at room temperature for 12 hr. The mixture was concentrated under reduced pressure. The residue was diluted with water. The solid formed was filtered off and dried on air to afford 3-fluoro-4-[4-(trifluoromethyl)-1H-imidazol-2-yl]benzonitrile (6.00 g, 23.5 mmol, 70.1% yield) as a yellow solid which was used in the next steps without further purification.

LCMS(ESI): [M+H]+ m/z: calcd 256.06; found 256.0.

### Step 2: The synthesis of 3-fluoro-4-[I-methyl-4-(trifluoromethyl)imidazol-2-yl]benzonitrile

3-fluoro-4-[4-(trifluoromethyl)-1H-imidazol-2-yl]benzonitrile (3.00 g, 11.8 mmol), cesium carbonate (5.75 g, 17.6 mmol) and dimethylsulfate (1.71 g, 13.5 mmol, 1.28 mL) were mixed in ACN (20 mL) under ice cooling. The reaction mixture was stirred and allowed to warm to room temperature for 24 hr. The reaction mixture was concentrated under reduced pressure. The resulting residue was diluted with water, the resulting solids were collected by filtration, dried on air and then recrystallized from iPrOH (10 mL) to afford 3-fluoro-4-[1-methyl-4-(trifluoromethyl)imidazol-2-yl]benzonitrile (1.35 g, 5.01 mmol, 42.7% yield) as a yellow solid.

¹H NMR (500 MHz, CDCl3) δ 3.68 (s, 3H), 7.41 (s, 1H), 7.52 (d, 1H), 7.60 (d, 1H), 7.75-7.81 (m, 1H).

LCMS(ESI): [M+H]+ m/z: calcd 270.08; found 270.2.

### Step 3: The synthesis of [3-fluoro-4-[1-methyl-4-(trifluoromethyl)imidazol-2-yl]phenyl]methanamine

A mixture of 3-fluoro-4-[1-methyl-4-(trifluoromethyl)imidazol-2-yl]benzonitrile (1.00 g, 3.71 mmol) and borane dimethyl sulfide complex (847 mg, 11.1 mmol, 1.06 mL) in THE (8.0 mL) was refluxed for 12 hr. The reaction mixture was cooled to room temperature and quenched by addition of MeOH (5 mL) followed by aqueous 6N hydrochloric acid (2.0 mL). The resulting mixture was concentrated under reduced pressure. To the resulting residue was treated with a solution of aqueous sodium hydroxide (2 mL, 40% wt.) and the mixture was extracted with MTBE (40 mL). The organic layer was dried over anhydrous sodium sulfate and concentrated under reduced pressure to afford [3-fluoro-4-[1-methyl-4-(trifluoromethyl)imidazol-2-yl]phenyl]methanamine (700 mg, 2.56 mmol, 69.0% yield) as a yellow oil which was used in the next steps without further purification.

LCMS(ESI): [M+H]+ m/z: calcd 274.1; found 274.0.

### Step 4: The synthesis of 2-chloro-N4-[[3-fluoro-4-[1-methyl-4-(trifluoromethyl)imidazol-2-yl]phenyl]methyl]pyrimidine-4,5-diamine

[3-fluoro-4-[1-methyl-4-(trifluoromethyl)imidazol-2-yl]phenyl]methanamine (700 mg, 2.56 mmol), 2,4-dichloropyrimidin-5-amine (588.20 mg, 3.59 mmol) and DIPEA (464 mg, 3.59 mmol, 625 μL) were mixed in DMSO (3.0 mL). The reaction mixture was stirred at 90° C. for 12 hr. The reaction mixture was cooled to room temperature and diluted with water. The resulting solids were collected by filtration, washed with water and dried on air to afford 2-chloro-N4-[[3-fluoro-4-[1-methyl-4-(trifluoromethyl)imidazol-2-yl]phenyl]methyl]pyrimidine-4,5-diamine (1.00 g, 2.50 mmol, 97.40% yield) as a brown solid which was used in the next steps without further purification.

LCMS(ESI): [M+H]+ m/z: calcd 401.09; found 401.0.

### Step 5: The synthesis of 2-chloro-9-(3-fluoro-4-(1-methyl-4-(trifluoromethyl)-1H-imidazol-2-yl)benzyl)-7H-purin-8(9H)-imine

A solution of KCN (813 mg, 12.5 mmol) in water (2.0 mL) was added dropwise to a stirred solution of bromine (2.00 g, 12.5 mmol, 644 μL) in MeOH (20 mL) at room temperature. The reaction mixture was stirred at room temperature for 15 min. The reaction mixture was added to a solution of 2-chloro-N4-[[3-fluoro-4-[1-methyl-4-(trifluoromethyl)imidazol-2-yl]phenyl]methyl]pyrimidine-4,5-diamine (500 mg, 1.25 mmol) in MeOH (10 mL). The resulting mixture was stirred at room temperature for 12 hr. The reaction mixture was concentrated under reduced pressure. The resulting residue was treated with an aqueous solution of potassium carbonate (50 mL, 10%). The resulting solid was collected by filtration, washed with water and dried on air to afford 2-chloro-9-[[3-fluoro-4-[1-methyl-4-(trifluoromethyl)imidazol-2-yl]phenyl]methyl]-7H-purin-8-imine (450 mg, 1.06 mmol, 84.7% yield) as a yellow solid which was used in the next steps without further purification.

LCMS(ESI): [M+H]+ m/z: calcd 426.10; found 426.0.

### Step 6: The synthesis of 2-(4-cyclopropyl-6-methoxy-pyrimidin-5-yl)-9-[[3-fluoro-4-[I-methyl-4-(trifluoromethyl)imidazol-2-yl]phenyl]methyl]-7H-purin-8-imine

2-chloro-9-[[3-fluoro-4-[1-methyl-4-(trifluoromethyl)imidazol-2-yl]phenyl]methyl]-7H-purin-8-imine (330 mg, 775 μmol), (4-cyclopropyl-6-methoxy-pyrimidin-5-yl)boronic acid (301 mg, 1.55 mmol), potassium phosphate tribasic (411 mg, 1.94 mmol) and XPhos Pd G3 (45.9 mg, 54.3 μmol) were mixed in a degassed mixture of dioxane (8.0 mL) and water (80 μL) under an argon atmosphere. The reaction mixture was stirred at 85° C. for 72 hr. The reaction mixture was cooled to room temperature and SiliaMetS® Dimercaptotriazine (100 mg) was added. The resulting mixture was stirred at room temperature for 3 hr. The mixture was concentrated under reduced pressure. The resulting residue was dissolved in EtOAc and filtered through a pad of silica, washing with MeOH. The filtrate was concentrated under reduced pressure to afford 2-(4-cyclopropyl-6-methoxy-pyrimidin-5-yl)-9-[[3-fluoro-4-[1-methyl-4-(trifluoromethyl)imidazol-2-yl]phenyl]methyl]-7H-purin-8-imine (140 mg, 260 μmol, 33.5% yield) as a yellow solid which was used in the next steps without further purification.

LCMS(ESI): [M+H]+ m/z: calcd 540.19; found 540.2.

### Step 7: The synthesis of 2-(4-cyclopropyl-6-methoxypyrimidin-5-yl)-9-(3-fluoro-4-(1-methyl-4-(trifluoromethyl)-1H-imidazol-2-yl)benzyl)-7-(2,2,2-trifluoroethyl)-7H-purin-8(9H)-imine

Cesium carbonate (186 mg, 571 μmol) was added to a solution of 2-(4-cyclopropyl-6-methoxy-pyrimidin-5-yl)-9-[[3-fluoro-4-[1-methyl-4-(trifluoromethyl)imidazol-2-yl]phenyl]methyl]-7H-purin-8-imine (140 mg, 260 μmol) in ACN (10 mL). The reaction mixture was stirred at room temperature for 15 min. 2,2,2-trifluoroethyl trifluoromethanesulfonate (102 mg, 441 μmol) was then added to the reaction mixture. The resulting mixture was stirred at 70° C. for 12 hr. The reaction mixture was cooled to room temperature and concentrated under reduced pressure. The residue was subjected to HPLC (gradient elution: 2-15 min, 20-45% ACN+FA (0.1% vol.); flow: 30 mL/min, column: Waters SunFire C18, 100×19 mm, 5 μm) to afford 2-(4-cyclopropyl-6-methoxypyrimidin-5-yl)-9-(3-fluoro-4-(1-methyl-4-(trifluoromethyl)-1H-imidazol-2-yl)benzyl)-7-(2,2,2-trifluoroethyl)-7H-purin-8(9H)-imine (4.00 mg, 6.44 μmol, 2.48% yield) as a yellow solid.

¹H NMR (600 MHz, DMSO-d6) δ 0.80-0.86 (m, 2H), 0.97-1.02 (m, 2H), 1.67-1.73 (m, 1H), 3.56 (s, 3H), 3.81 (s, 3H), 4.75-5.02 (m, 2H), 5.10-5.26 (m, 2H), 7.09-7.14 (m, 1H), 7.30-7.45 (m, 2H), 7.52-7.58 (m, 1H), 7.97 (s, 1H), 8.31-8.38 (m, 1H), 8.62 (s, 1H).

LCMS(ESI): [M+H]+ m/z: calcd 622.22; found 622.0.

### Example T-140

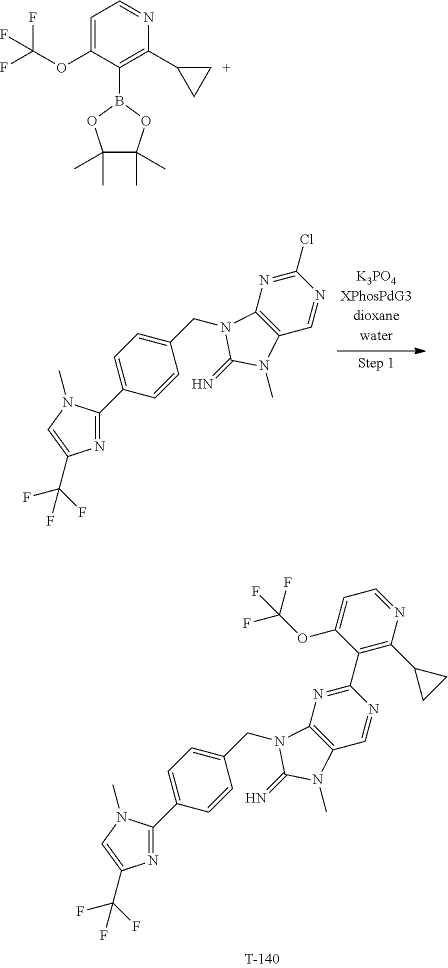

### Step 1: The synthesis of 2-[2-cyclopropyl-4-(trifluoromethoxy)-3-pyridyl]-7-methyl-9-[[4-[1-methyl-4-(trifluoromethyl)imidazol-2-yl]phenyl]methyl]purin-8-imine

2-Chloro-7-methyl-9-[[4-[1-methyl-4-(trifluoromethyl)imidazol-2-yl]phenyl]methyl]purin-8-imine (192 mg, 456 μmol), 2-cyclopropyl-3-(4,4,5,5-tetramethyl-1,3,2-dioxaborolan-2-yl)-4-(trifluoromethoxy)pyridine (150 mg, 456 μmol) and potassium phosphate tribasic (194 mg, 912 μmol) were mixed in a degassed mixture of dioxane (3.0 mL) and water (300 μL) under argon atmosphere. The reaction mixture was stirred at 85° C. for 12 hr. The reaction mixture was cooled to room temperature. SiliaMetS®Dimercaptotriazine (100 mg) was added to the reaction mixture. The resulting mixture was stirred at room temperature for 3 hr. The mixture was diluted with MTBE (5.0 mL) and filtered through a pad of silica gel. The filtrate was concentrated under reduced pressure. The residue was subjected to HPLC (gradient elution: 2-10 min., 20-50% ACN, +0.1% vol. of 25% aq. NH3, flow: 30 mL/min, column: SunFire C18 100×19 mm, 5 μm) to afford 2-[2-cyclopropyl-4-(trifluoromethoxy)-3-pyridyl]-7-methyl-9-[[4-[1-methyl-4-(trifluoromethyl)imidazol-2-yl]phenyl]methyl]purin-8-imine (4.00 mg, 6.80 μmol, 1.49% yield) as a yellow solid.

¹H NMR (600 MHz, DMSO-d6) δ 0.76-0.82 (m, 2H), 1.04-1.09 (m, 2H), 1.72-1.79 (m, 1H), 3.41 (s, 3H), 3.76 (s, 3H), 5.08-5.21 (m, 2H), 6.27-6.51 (m, 1H), 7.06-7.11 (m, 1H), 7.53-761 (m, 4H), 7.68 (s, 1H), 8.09-8.19 (m, 1H), 8.47 (d, 1H).

LCMS(ESI): [M+H]+ m/z: calcd 589.22; found 589.2.

### Example T-106

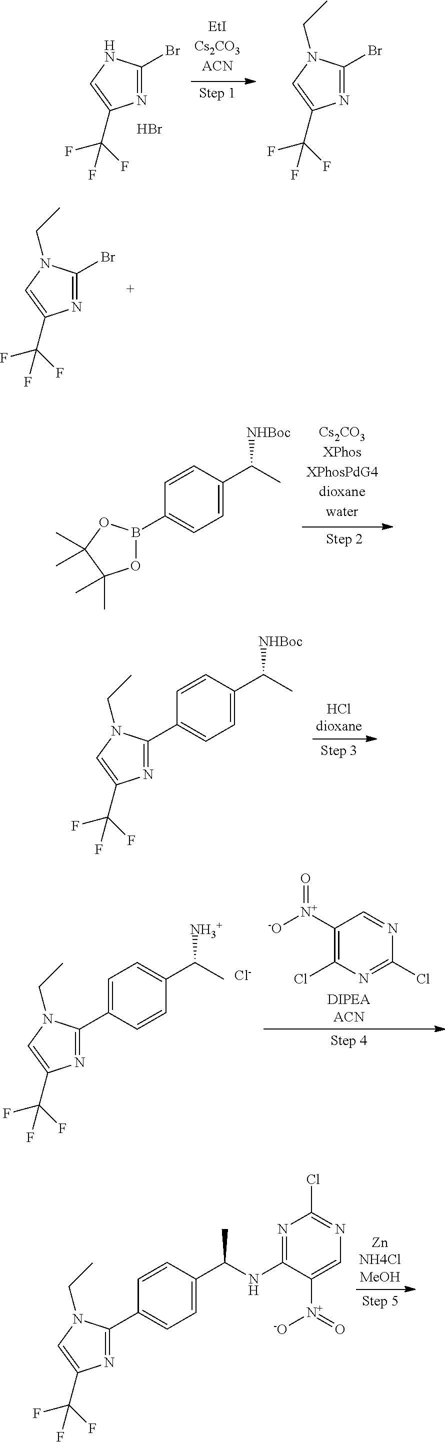

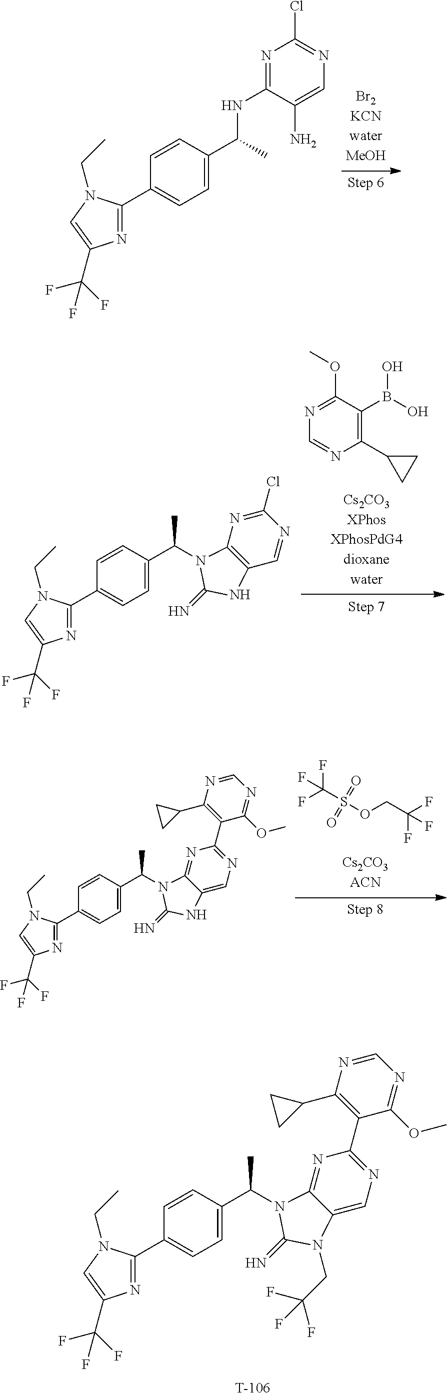

### Step 1: The synthesis of 2-bromo-1-ethyl-4-(trifluoromethyl)-1H-imidazole

Cesium carbonate (2.75 g, 8.45 mmol) was added to a solution of 2-bromo-5-(trifluoromethyl)-1H-imidazole hydrobromide (1.00 g, 3.38 mmol) in ACN (19.7 mL). The resulting mixture was cooled with ice. A solution of iodoethane (580 mg, 3.72 mmol, 299 μL) in THE (5.0 mL) was added dropwise to the mixture. The reaction mixture was stirred at room temperature for 3 hr. The solids were filtered out. The filtrate was concentrated under reduced pressure to afford 2-bromo-1-ethyl-4-(trifluoromethyl)-1H-imidazole (800 mg, 3.29 mmol, 97.4% yield) as a light-yellow solid which was used in the next steps without further purification.

¹H NMR (500 MHz, DMSO-d6) δ 1.33 (t, 3H), 4.01 (q, 2H), 8.09 (s, 1H).

LCMS(ESI): [M+H]+ m/z: calcd 242.99; found 243.0.

### Step 2: The synthesis of (R)-tert-butyl (1-(4-(1-ethyl-4-(trifluoromethyl)-1H-imidazol-2-yl)phenyl)ethyl)carbamate

2-bromo-1-ethyl-4-(trifluoromethyl)imidazole (800 mg, 3.29 mmol), tert-butyl N-[(1R)-1-[4-(4,4,5,5-tetramethyl-1,3,2-dioxaborolan-2-yl)phenyl]ethyl]carbamate (1.14 g, 3.29 mmol), XPhos Pd G4 (212 mg, 247 μmol) and cesium carbonate (2.68 g, 8.23 mmol) were mixed in a degassed mixture of dioxane (25 mL) and water (5.0 mL) under argon atmosphere at room temperature. The reaction mixture was stirred at 90° C. for 12 hr. The reaction mixture was cooled to room temperature and concentrated under reduced pressure. The residue was diluted with EtOAc (20 mL) and washed with water (20 mL). The organic layer was dried over anhydrous sodium sulfate and filtered through a thin pad of silica gel. The filtrate was concentrated under reduced pressure to afford (R)-tert-butyl (1-(4-(1-ethyl-4-(trifluoromethyl)-1H-imidazol-2-yl)phenyl)ethyl)carbamate (900 mg, 2.35 mmol, 71.4% yield) as a black solid which was used in the next steps without further purification.

LCMS(ESI): [M+H]+ m/z: calcd 384.23; found 384.4.

### Step 3: The synthesis of (R)-1-(4-(1-ethyl-4-(trifluoromethyl)-1H-imidazol-2-yl)phenyl)ethanaminium chloride

Tert-butyl N-[(1R)-1-[4-[1-ethyl-4-(trifluoromethyl)imidazol-2-yl]phenyl]ethyl]carbamate (900 mg, 2.35 mmol) was dissolved in a solution of hydrogen chloride in dioxane (1.1 mL, 4.0 M). The reaction mixture was stirred at room temperature for 12 hr. The reaction mixture was concentrated under reduced pressure to afford (R)-1-(4-(1-ethyl-4-(trifluoromethyl)-1H-imidazol-2-yl)phenyl)ethanaminium chloride (650 mg, 2.03 mmol, 86.6% yield) as a grey solid which was used directly in the next step without further purification.

LCMS(ESI): [M+H]+ m/z: calcd 284.17; found 284.0.

### Step 4: The synthesis of (R)-2-chloro-N-(1-(4-(1-ethyl-4-(trifluoromethyl)-1H-imidazol-2-yl)phenyl)ethyl)-5-nitropyrimidin-4-amine

DIPEA (788 mg, 6.10 mmol, 1.06 mL) and 2,4-dichloro-5-nitropyrimidine (394 mg, 2.03 mmol) were added to a stirred solution of (R)-1-(4-(1-ethyl-4-(trifluoromethyl)-1H-imidazol-2-yl)phenyl)ethanaminium chloride (650 mg, 2.03 mmol) in ACN (20 mL) under argon atmosphere. The reaction mixture was stirred at 90° C. for 12 hr. The reaction mixture was cooled to room temperature, diluted with DCM (20 mL) and washed with water (2×10 mL). The organic layer was dried over anhydrous sodium sulfate and concentrated under reduced pressure to afford (R)-2-chloro-N-(1-(4-(1-ethyl-4-(trifluoromethyl)-1H-imidazol-2-yl)phenyl)ethyl)-5-nitropyrimidin-4-amine (800 mg, 1.81 mmol, 89.3% yield) as a brown gum which was used in the next steps without further purification.

LCMS(ESI): [M+H]+ m/z: calcd 441.12; found 441.0.

### Step 5: The synthesis of (R)-2-chloro-N4-(1-(4-(I-ethyl-4-(trifluoromethyl)-1H-imidazol-2-yl)phenyl)ethyl)pyrimidine-4,5-diamine

2-Chloro-5-nitro-N-[(1R)-1-[4-[1-ethyl-4-(trifluoromethyl)imidazol-2-yl]phenyl]ethyl]pyrimidin-4-amine (800 mg, 1.81 mmol) and ammonium chloride (1.16 g, 21.8 mmol) were mixed in MeOH (30 mL). The resulting mixture was cooled to 0° C. Zinc powder (712 mg, 10.9 mmol) was added to the stirred mixture. The reaction mixture was stirred at −10° C. for 1 hr. The solids were filtered out. The filtrate was concentrated under reduced pressure. The residue was diluted with EtOAc (50 mL) and filtered. The filtrate was concentrated under reduced pressure to afford (R)-2-chloro-N4-(1-(4-(1-ethyl-4-(trifluoromethyl)-1H-imidazol-2-yl)phenyl)ethyl)pyrimidine-4,5-diamine (700 mg, 1.70 mmol, 93.9% yield) as a grey solid which was used in the next steps without further purification.

LCMS(ESI): [M+H]+ m/z: calcd 411.16; found 411.0.

### Step 6: The synthesis of (R)-2-chloro-9-(1-(4-(I-ethyl-4-(trifluoromethyl)-1H-imidazol-2-yl)phenyl)ethyl)-7H-purin-8(9H)-imine

A solution of potassium cyanide (1.02 g, 15.7 mmol) in water (1.0 mL) was added to a precooled to 0° C. stirred solution of Br2 (2.48 g, 15.5 mmol) in water (15 mL). The resulting mixture was stirred at 0° C. for 15 min. A solution of 2-chloro-N4-[rac-(1R)-1-[4-[1-ethyl-4-(trifluoromethyl)imidazol-2-yl]phenyl]ethyl]pyrimidine-4,5-diamine (700 mg, 1.70 mmol) in MeOH (35 mL) was added to the mixture at 0° C. The reaction mixture was allowed to warm to room temperature then heated to 40° C. and stirred at 40° C. temperature for 12 hr. The resulting mixture was cooled to room temperature and concentrated under reduced pressure to 1/3 of the volume. The solid precipitate was filtered off to afford 0.22 g of the product. The filtrate was extracted with EtOAc (2×15 mL). The combined organic layers were concentrated under reduced pressure. The residue was combined with the filter cake to afford (R)-2-chloro-9-(1-(4-(1-ethyl-4-(trifluoromethyl)-1H-imidazol-2-yl)phenyl)ethyl)-7H-purin-8(9H)-imine (0.52 g, 1.19 mmol, 70.0% yield) as a brown solid which was used in the next steps without further purification.

LCMS(ESI): [M+H]+ m/z: calcd 436.15; found 436.0.

### Step 7: The synthesis of (R)-2-(4-cyclopropyl-6-methoxypyrimidin-5-yl)-9-(1-(4-(1-ethyl-4-(trifluoromethyl)-1H-imidazol-2-yl)phenyl)ethyl)-7H-purin-8(9H)-imine

2-Chloro-9-[(1R)-1-[4-[1-ethyl-4-(trifluoromethyl)imidazol-2-yl]phenyl]ethyl]-7H-purin-8-imine (300 mg, 688 μmol), (4-cyclopropyl-6-methoxypyrimidin-5-yl)boronic acid (200 mg, 1.03 mmol), XPhos Pd G4 (44.4 mg, 51.6 μmol), XPhos (24.6 mg, 51.6 μmol) and cesium carbonate (561 mg, 1.72 mmol) were mixed in a degassed mixture of dioxane (10 mL) and water (2.0 mL) under argon atmosphere at room temperature. The reaction mixture was stirred at 95° C. for 12 hr. The reaction mixture was cooled to room temperature and concentrated under reduced pressure. The residue was extracted with EtOAc (20 mL) and concentrated under reduced pressure to afford (R)-2-(4-cyclopropyl-6-methoxypyrimidin-5-yl)-9-(1-(4-(1-ethyl-4-(trifluoromethyl)-1H-imidazol-2-yl)phenyl)ethyl)-7H-purin-8(9H)-imine (300 mg, 546 μmol, 79.4% yield) as a brown gum which was used in the next step without further purification.

LCMS(ESI): [M+H]+ m/z: calcd 550.26; found 550.0.

### Step 8: The synthesis of (R)-2-(4-cyclopropyl-6-methoxypyrimidin-5-yl)-9-(1-(4-(I-ethyl-4-(trifluoromethyl)-1H-imidazol-2-yl)phenyl)ethyl)-7-(2,2,2-trifluoroethyl)-7H-purin-8(9H)-imine

Cesium carbonate (534 mg, 1.64 mmol) was added to a solution of 2-(4-cyclopropyl-6-methoxy-pyrimidin-5-yl)-9-[(1R)-1-[4-[1-ethyl-4-(trifluoromethyl)imidazol-2-yl]phenyl]ethyl]-7H-purin-8-imine (300 mg, 546 μmol) in ACN (10.0 mL). The resulting mixture was heated to 80° C. A solution of 2,2,2-trifluoroethyl trifluoromethanesulfonate (253 mg, 1.09 mmol, 157 μL) in ACN (5.0 mL) was added dropwise to the mixture. The reaction mixture was stirred at 80° C. for 20 hr. The reaction mixture was cooled to room temperature and filtered. The filtrate was concentrated under reduced pressure. The residue was subjected to HPLC (0-6 min., 30-50% water—ACN, +0.1% vol. of 25% aq. NH3, flow: 30 mL/min, column: XBridge C18 100×19 mm, 5 μm), then repurified by HPLC (0-5 min., 40-90% water—MeOH, +0.1% vol. of 25% aq. NH3, flow: 30 mL/min, column: XBridge C18 100×19 mm, 5 μm) to afford (R)-2-(4-cyclopropyl-6-methoxypyrimidin-5-yl)-9-(1-(4-(1-ethyl-4-(trifluoromethyl)-1H-imidazol-2-yl)phenyl)ethyl)-7-(2,2,2-trifluoroethyl)-7H-purin-8(9H)-imine (12.3 mg, 19.5 μmol, 3.57% yield) as a brown gum.

¹H NMR (600 MHz, DMSO-d6) δ 0.76-0.87 (m, 2H), 0.95-1.03 (m, 2H), 1.30 (t, 3H), 1.68-1.73 (m, 1H), 1.97 (d, 3H), 3.83 (s, 3H), 4.05 (q, 2H), 4.78-5.03 (m, 2H), 5.84-5.94 (m, 1H), 6.90-7.16 (m, 1H), 7.53-7.63 (m, 4H), 8.01 (s, 1H), 8.30-8.38 (m, 1H), 8.62 (s, 1H).

LCMS(ESI): [M+H]+ m/z: calcd 632.27; found 632.2.

### Example T-130

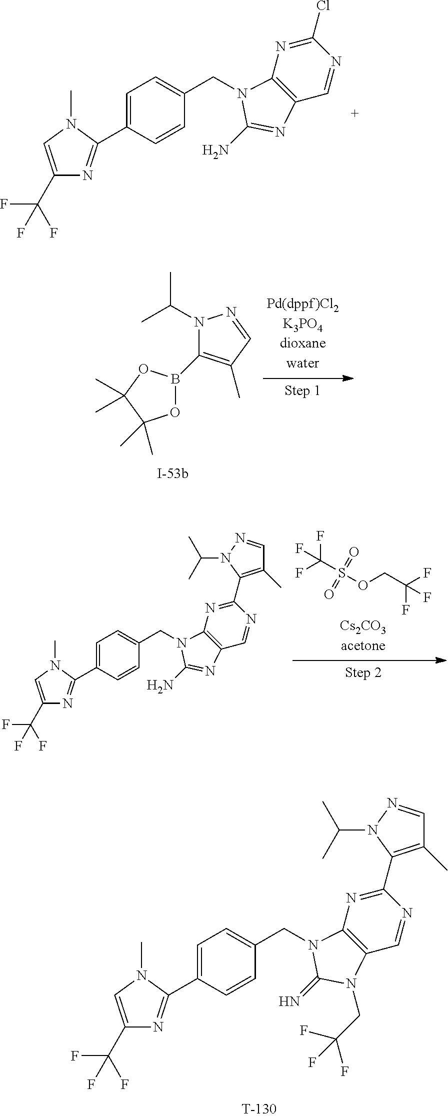

The synthesis of 1-isopropyl-4-methyl-5-(4,4,5,5-tetramethyl-1,3,2-dioxaborolan-2-yl)pyrazole (I-53b) is described by Intermediate 53.

### Step 1: The synthesis of 2-(2-isopropyl-4-methyl-pyrazol-3-yl)-9-[[4-[I-methyl-4-(trifluoromethyl)imidazol-2-yl]phenyl]methyl]purin-8-amine

2-chloro-9-[[4-[1-methyl-4-(trifluoromethyl)imidazol-2-yl]phenyl]methyl]purin-8-amine (500 mg, 1.23 mmol), 1-isopropyl-4-methyl-5-(4,4,5,5-tetramethyl-1,3,2-dioxaborolan-2-yl)pyrazole (460 mg, 1.84 mmol), bis(diphenylphosphino)ferrocene]dichloropalladium(II)-DCM (100 mg, 123 μmol) and potassium phosphate tribasic (781 mg, 3.68 mmol) were mixed in a degassed mixture of dioxane (7.0 mL) and water (700 μL) under argon atmosphere. The reaction mixture was stirred at 75° C. for 12 hr under argon atmosphere. The reaction mixture was cooled to room temperature and diluted with EtOAc (10 mL). Anhydrous Na2SO4 and SiliaMetS® Dimercaptotriazine (200 mg) were added to the mixture and the resulting mixture was stirred for 1 hr. The solids were filtered out. The filtrate was concentrated under reduced pressure. The residue was subjected to flash column chromatography (SiO2, gradient chloroform—methanol) to afford 2-(2-isopropyl-4-methyl-pyrazol-3-yl)-9-[[4-[1-methyl-4-(trifluoromethyl)imidazol-2-yl]phenyl]methyl]purin-8-amine (550 mg, 1.11 mmol, 90.5% yield) as a black foam which was used in the next step without further purification.

LCMS(ESI): [M+H]+ m/z: calcd 496.25; found 496.2.

### Step 2: The synthesis of 2-(2-isopropyl-4-methyl-pyrazol-3-yl)-9-[[4-[I-methyl-4-(trifluoromethyl)imidazol-2-yl]phenyl]methyl]-7-(2,2,2-trifluoroethyl)purin-8-imine

2-(2-isopropyl-4-methyl-pyrazol-3-yl)-9-[[4-[1-methyl-4-(trifluoromethyl)imidazol-2-yl]phenyl]methyl]purin-8-amine (550 mg, 1.11 mmol), 2,2,2-trifluoroethyl trifluoromethanesulfonate (258 mg, 1.11 mmol) and cesium carbonate (723 mg, 2.22 mmol) were mixed in acetone (20 mL). The reaction mixture was stirred at room temperature for 12 hr, then solids were filtered out. The filtrate was subjected to HPLC (0.5-6.5 min., 40-75% water—ACN, +0.1% vol. of 25% aq. NH3, flow: 30 mL/min, column: XBridge C18 100×19 mm, 5 μm) to afford 2-(2-isopropyl-4-methyl-pyrazol-3-yl)-9-[[4-[1-methyl-4-(trifluoromethyl)imidazol-2-yl]phenyl]methyl]-7-(2,2,2-trifluoroethyl)purin-8-imine (15.0 mg, 26.0 μmol, 2.34% yield) as an off-white solid.

¹H NMR (600 MHz, DMSO-d6) δ 1.26-1.32 (m, 6H), 2.07-2.12 (m, 3H), 3.73 (s, 3H), 4.78-5.03 (m, 2H), 5.11-5.28 (m, 3H), 7.09-7.14 (m, 1H), 7.30 (s, 1H), 7.43-7.52 (m, 2H), 7.65-7.71 (m, 2H), 7.90 (s, 1H), 8.30-8.37 (m, 1H).

LCMS(ESI): [M+H]+ m/z: calcd 578.26; found 578.2.

### Example T-126

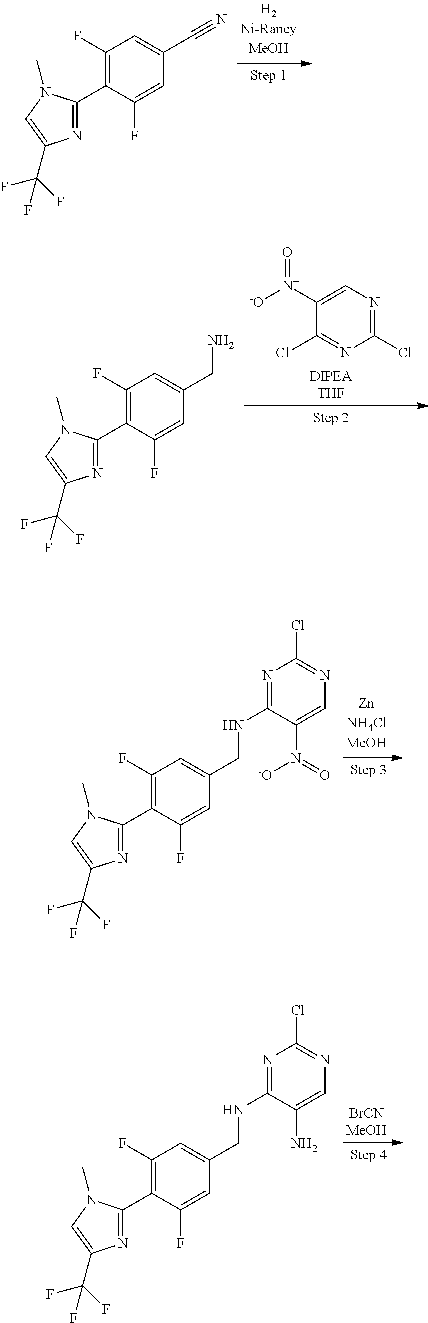

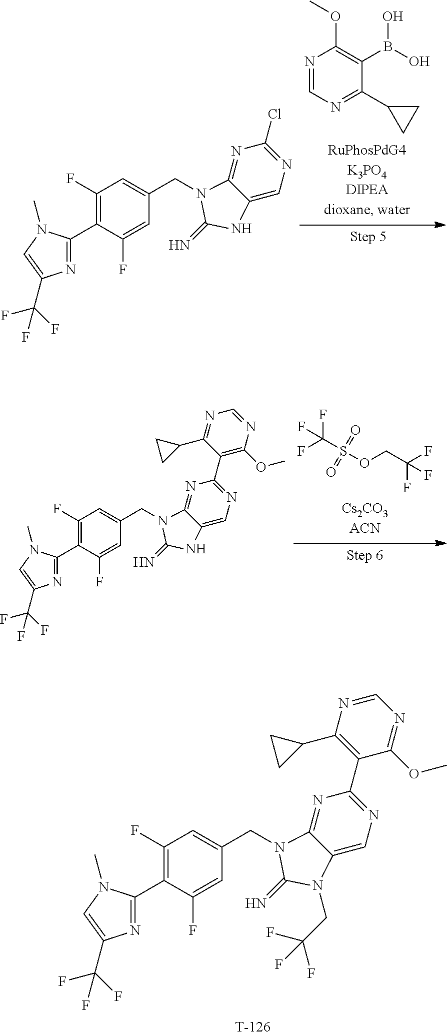

### Step 1: The synthesis of[3,5-difluoro-4-[I-methyl-4-(trifluoromethyl)imidazol-2-yl]phenyl]methanamine

Step 1 is given by Intermediate 31.

### Step 2: The synthesis of 2-chloro-N-[[3,5-difluoro-4-[-methyl-4-(trifluoromethyl)imidazol-2-yl]phenyl]methyl]-5-nitro-pyrimidin-4-amine

A solution of [3,5-difluoro-4-[1-methyl-4-(trifluoromethyl)imidazol-2-yl]phenyl]methanamine (900 mg, 3.09 mmol) and DIPEA (439 mg, 3.40 mmol, 592 μL) in THE (20 mL) was added to a stirred solution of 2,4-dichloro-5-nitro-pyrimidine (560 mg, 3.09 mmol) in THE (10 mL). The reaction mixture was stirred at room temperature for 16 hr. The reaction mixture was diluted with EtOAc (50 mL) and washed with water (20 mL) and brine (10 mL). The organic layer was concentrated under reduced pressure to afford 2-chloro-N-[[3,5-difluoro-4-[1-methyl-4-(trifluoromethyl)imidazol-2-yl]phenyl]methyl]-5-nitro-pyrimidin-4-amine (1.20 g, 2.67 mmol, 86.3% yield) as a yellow oil which was used in the next steps without further purification.

¹H NMR (400 MHz, DMSO-d6) δ 3.57 (s, 3H), 4.85 (d, 2H), 7.34 (d, 2H), 8.06 (s, 1H), 9.08 (s, 1H), 9.67 (t, 1H).

LCMS(ESI): [M+H]+ m/z: calcd 449.06; found 449.0.

### Step 3: The synthesis of 2-chloro-N4-[[3,5-difluoro-4-[-methyl-4-(trifluoromethyl)imidazol-2-yl]phenyl]methyl]pyrimidine-4,5-diamine

Ammonium chloride (1.74 g, 32.5 mmol) and zinc powder (850 mg, 13.0 mmol) were added to a stirred solution of 2-chloro-N-[[3,5-difluoro-4-[1-methyl-4-(trifluoromethyl)imidazol-2-yl]phenyl]methyl]-5-nitro-pyrimidin-4-amine (1.20 g, 2.67 mmol) in MeOH (60 mL). The reaction mixture was stirred at room temperature for 16 hr. The reaction mixture was filtered. The filtrate was concentrated under reduced pressure. The residue was diluted with EtOAc (50 mL) and washed with water (20 mL) and brine (20 mL). The organic layer was concentrated under reduced pressure to afford 2-chloro-N4-[[3,5-difluoro-4-[1-methyl-4-(trifluoromethyl)imidazol-2-yl]phenyl]methyl]pyrimidine-4,5-diamine (1.00 g, 2.39 mmol, 86.5% yield) as a yellow solid which was used in the next steps without further purification.

LCMS(ESI): [M+H]+ m/z: calcd 419.08; found 419.0.

### Step 4: The synthesis of 2-chloro-9-[[3,5-difluoro-4-[I-methyl-4-(trifluoromethyl)imidazol-2-yl]phenyl]methyl]-7H-purin-8-imine

Cyanogen bromide (1.83 g, 17.3 mmol) was added to a stirred solution of 2-chloro-N4-[[3,5-difluoro-4-[1-methyl-4-(trifluoromethyl)imidazol-2-yl]phenyl]methyl]pyrimidine-4,5-diamine (1.81 g, crude) in MeOH (20 mL). The reaction mixture was stirred at 40° C. for 48 hr. The reaction mixture was cooled to room temperature and concentrated under reduced pressure. The residue was diluted with MTBE (60 mL), quenched with a solution of aqueous NaHCO3 to pH≈8-9 and extracted with EtOAc (100 mL). The organic layer was washed with water (50 mL) and brine (50 mL) and concentrated under reduced pressure. The residue was subjected to flash column chromatography (SiO2, gradient ACN-MeOH) to afford 2-chloro-9-[[3,5-difluoro-4-[1-methyl-4-(trifluoromethyl)imidazol-2-yl]phenyl]methyl]-7H-purin-8-imine (450 mg, 1.01 mmol, 29.3% yield) as a brown solid.

¹H NMR (500 MHz, DMSO-d6) δ 3.54 (s, 3H), 5.36 (s, 2H), 7.14 (d, 2H), 7.50 (s, 2H), 8.05 (s, 1H), 8.31 (s, 1H).

LCMS(ESI): [M+H]+ m/z: calcd 444.08; found 444.0.

### Step 5: The synthesis of 2-(4-cyclopropyl-6-methoxy-pyrimidin-5-yl)-9-[[3,5-difluoro-4-[1-methyl-4-(trifluoromethyl)imidazol-2-yl]phenyl]methyl]-7H-purin-8-imine

2-chloro-9-[[3,5-difluoro-4-[1-methyl-4-(trifluoromethyl)imidazol-2-yl]phenyl]methyl]-7H-purin-8-imine (450 mg, 1.01 mmol), (4-cyclopropyl-6-methoxy-pyrimidin-5-yl)boronic acid (256 mg, 1.32 mmol) and DIPEA (275 mg, 2.13 mmol, 371 μL) were mixed in dioxane (10 mL) and water (500 μL). The resulting mixture was degassed. RuPhos Pd G4 (43.1 mg, 50.7 μmol) was added to the mixture. The reaction mixture was stirred at 85° C. for 16 hr. under argon atmosphere. (4-cyclopropyl-6-methoxy-pyrimidin-5-yl)boronic acid (256 mg, 1.32 mmol) and RuPhos Pd G4 (43.1 mg, 50.7 μmol) were added to the reaction mixture. The resulting mixture was stirred at 85° C. for 16 hr under argon atmosphere. The reaction mixture was cooled to room temperature, diluted with EtOAc (20 mL) and washed with water (2×10 mL). The organic layer was concentrated under reduced pressure. The residue was subjected to HPLC (0.5-6.5 min, 10-60% water+FA (0.1% vol.)—ACN+FA (0.1% vol.); flow: 30 mL/min, column: SunFire C18, 100×19 mm, 5 μm) to afford 2-(4-cyclopropyl-6-methoxy-pyrimidin-5-yl)-9-[[3,5-difluoro-4-[1-methyl-4-(trifluoromethyl)imidazol-2-yl]phenyl]methyl]-7H-purin-8-imine (230 mg, 413 μmol, 40.7% yield) as a white solid.

LCMS(ESI): [M+H]+ m/z: calcd 558.20; found 558.2.

### Step 6: The synthesis of 2-(4-cyclopropyl-6-methoxy-pyrimidin-5-yl)-9-[[3,5-difluoro-4-[1-methyl-4-(trifluoromethyl)imidazol-2-yl]phenyl]methyl]-7-(2,2,2-trifluoroethyl)purin-8-imine

2,2,2-Trifluoroethyl trifluoromethanesulfonate (22.1 mg, 95.2 μmol, 13.7 μL) was added to a stirred solution of 2-(4-cyclopropyl-6-methoxy-pyrimidin-5-yl)-9-[[3,5-difluoro-4-[1-methyl-4-(trifluoromethyl)imidazol-2-yl]phenyl]methyl]-7H-purin-8-imine (72.0 mg, 86.5 μmol) and cesium carbonate (42.3 mg, 130 μmol) in ACN (2.0 mL). The reaction mixture was stirred at 50° C. for 16 hr. The reaction mixture was cooled to room temperature and concentrated under reduced pressure. The residue was diluted with EtOAc (20 mL), washed with water (10 mL) and brine (10 mL). To the obtained solution SiliaMetS® Dimercaptotriazine (50 mg) was added and the resulting mixture was stirred for 1 hr. The mixture was filtered. The filtrate was concentrated under reduced pressure. The residue was subjected to HPLC (0.5-6.5 min, 35-50% water+FA (0.1% vol.)—ACN+FA (0.1% vol.); flow: 30 mL/min, column: SunFire C18, 100×19 mm, 5 μm) to afford 2-(4-cyclopropyl-6-methoxy-pyrimidin-5-yl)-9-[[3,5-difluoro-4-[1-methyl-4-(trifluoromethyl)imidazol-2-yl]phenyl]methyl]-7-(2,2,2-trifluoroethyl)purin-8-imine (4.00 mg, 6.25 μmol, 7.23 yield) as an off-white solid.

¹H NMR (600 MHz, DMSO-d6) δ 0.79-0.86 (m, 2H), 0.97-1.02 (m, 2H), 1.68-1.75 (m, 1H), 3.54 (s, 3H), 3.81 (s, 3H), 4.75-5.00 (m, 2H), 5.12-5.28 (m, 2H), 7.12 (s, 1H), 7.29-7.39 (m, 2H), 8.04 (s, 1H), 8.32-8.37 (m, 1H), 8.62 (s, 1H).

LCMS(ESI): [M+H]+ m/z: calcd 640.21; found 640.2.

### Example T-117

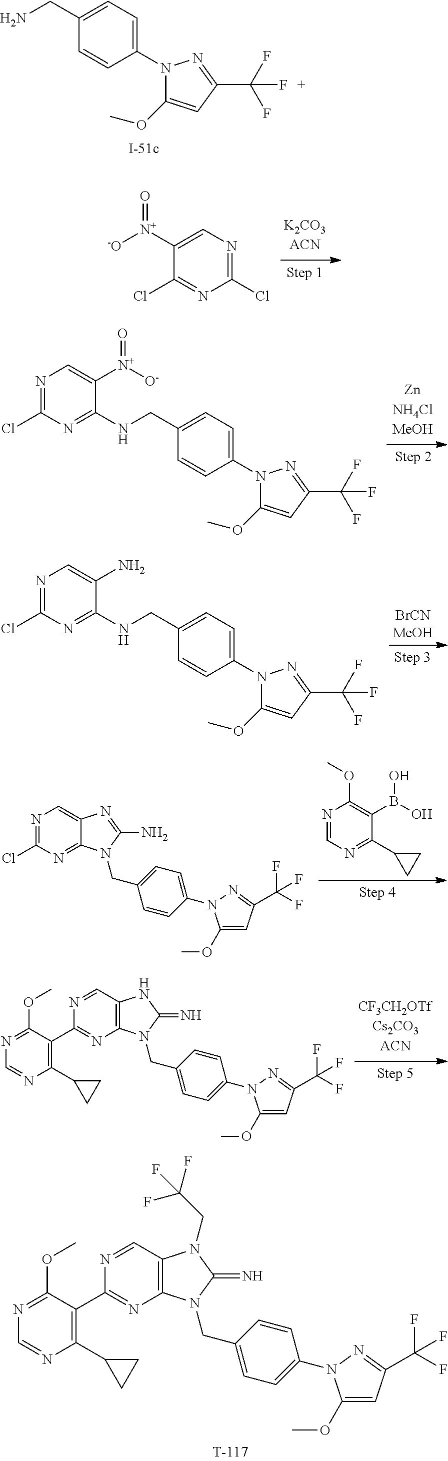

### Step 1: The synthesis of 2-chloro-N-[[4-[5-methoxy-3-(trifluoromethyl)pyrazol-1-yl]phenyl]methyl]-5-nitro-pyrimidin-4-amine

The synthesis of the starting (4-(5-methoxy-3-(trifluoromethyl)-1H-pyrazol-1-yl)phenyl)methanamine is described by Intermediate 51. [1140] 2,4-dichloro-5-nitro-pyrimidine (572 mg, 2.95 mmol) was dissolved in ACN (80 mL), and potassium carbonate (611 mg, 4.42 mmol) was added. The reaction mixture was stirred at room temperature for 5 minutes then the mixture was cooled to −15° C. and [4-[5-methoxy-3-(trifluoromethyl)pyrazol-1-yl]phenyl]methanamine (1.00 mg, 2.95 mmol) was added. The resulting mixture was stirred at ambient temperature for 18 hr. The mixture was filtered. The filtrate was concentrated under reduced pressure. The residue was diluted with water (20 mL) and extracted with EtOAc (2×30 mL). The combined organic layers were washed with brine (20 mL), dried over anhydrous sodium sulfate and concentrated under reduced pressure. The residue was subjected to flash-column chromatography (SiO2, gradient elution: hexane—EtOAc) to afford 2-chloro-N-[[4-[5-methoxy-3-(trifluoromethyl)pyrazol-1-yl]phenyl]methyl]-5-nitro-pyrimidin-4-amine (650 mg, 1.52 mmol, 51.4% yield) as an off-white solid.

¹H NMR (400 MHz, CDCl3) δ 4.00 (s, 3H), 4.86 (d, 2H), 5.94 (s, 1H), 7.43 (d, 2H), 7.71 (d, 2H), 8.64 (br, 1H), 9.07 (s, 1H).

LCMS(ESI): [M+H]+ m/z: calcd 429.06; found 429.0.

### Step 2: The synthesis of 2-chloro-N4-[[4-[5-methoxy-3-(trifluoromethyl)pyrazol-1-yl]phenyl]methyl]pyrimidine-4,5-diamine

2-chloro-N-[[4-[5-methoxy-3-(trifluoromethyl)pyrazol-1-yl]phenyl]methyl]-5-nitro-pyrimidin-4-amine (7.00 g, 16.3 mmol) and ammonium chloride (13.1 g, 245 mmol) were dissolved in MeOH (500 mL). The resulting solution was cooled to −10° C. then zinc (8.54 g, 131 mmol, dust) was added portionwise, keeping internal temperature near 0° C. The reaction mixture was allowed to warm and stirred at ambient temperature for 14 hr. The solids were filtered out. The filtrate was concentrated under reduced pressure. The residue was partitioned between DCM (600 mL) and water (250 ml). The organic layer was separated, washed with water (100 mL), dried over anhydrous sodium sulfate and concentrated under reduced pressure to afford 2-chloro-N4-[[4-[5-methoxy-3-(trifluoromethyl)pyrazol-1-yl]phenyl]methyl]pyrimidine-4,5-diamine (5.00 g, 12.54 mmol, 76.8% yield) as a brown solid which was used in the next steps without further purification.

LCMS(ESI): [M+H]+ m/z: calcd 399.1; found 399.2.

### Step 3: The synthesis of 2-chloro-9-[[4-[5-methoxy-3-(trifluoromethyl)pyrazol-1-yl]phenyl]methyl]-7H-purin-8-imine

2-chloro-N4-[[4-[5-methoxy-3-(trifluoromethyl)pyrazol-1-yl]phenyl]methyl]pyrimidine-4,5-diamine (5.00 g, 12.54 mmol) was dissolved in MeOH (250 mL). Cyanogen bromide (3.98 g, 37.6 mmol) was added portionwise to the stirred solution at room temperature. The reaction mixture was stirred at 40° C. for 72 hr in a capped flask. The resulting mixture was cooled to room temperature and concentrated under reduced pressure. The residue was triturated with MTBE (200 mL). The solids were collected by filtration then partitioned between EtOAc (400 mL) and saturated aqueous NaHCO3 solution (200 mL). The organic layer was separated, washed with brine, dried over anhydrous sodium sulfate and concentrated under reduced pressure to give a crude product (5.1 g, 71% purity by LCMS). The crude was purified by flash-column chromatography (SiO2, gradient acetonitrile—methanol) to afford 2-chloro-9-[[4-[5-methoxy-3-(trifluoromethyl)pyrazol-1-yl]phenyl]methyl]-7H-purin-8-imine (2.00 g, 4.72 mmol, 37.6% yield) as a light-yellow solid.

¹H NMR (500 MHz, DMSO-d6) δ 3.96 (s, 3H), 5.32 (s, 1H), 6.42 (s, 1H), 7.34 (d, 2H), 7.45 (br, 2H), 7.60 (d, 2H), 8.29 (s, 1H).

LCMS(ESI): [M+H]+m z: calcd 424.09; found 424.0.

NOTE: BrCN is highly toxic and volatile reagent therefore must be handled with extreme precautious. Reaction must be performed in closed reactor to avoid BrCN evaporation. At 40° C. methanol vapors doesn't cause significant inner pressure in the reactor but the factor of possible overpressure must be accounted for during reaction set-up.

### Step 4: The synthesis of 2-(4-cyclopropyl-6-methoxy-pyrimidin-5-yl)-9-[[4-[5-methoxy-3-(trifluoromethyl)pyrazol-1-yl]phenyl]methyl]purin-8-amine

2-chloro-9-[[4-[5-methoxy-3-(trifluoromethyl)pyrazol-1-yl]phenyl]methyl]purin-8-amine (550 mg, 1.30 mmol), (4-cyclopropyl-6-methoxy-pyrimidin-5-yl)boronic acid (378 mg, 1.95 mmol), potassium phosphate tribasic (689 mg, 3.24 mmol), XPhos Pd G3 (82.4 mg, 97.3 μmol) and XPhos (30.9 mg, 64.9 μmol) were mixed in degassed dioxane (10 mL) and water (1 mL) under argon atmosphere. The reaction mixture was stirred at 110° C. for 16 hr. The mixture was cooled to room temperature and the solvents were evaporated. The residue was diluted with water (20 mL) and extracted with EtOAc (3×50 mL), the combined organic layers were dried over anhydrous sodium sulfate and filtered. To the obtained filtrate was added SiliaMetS® Dimercaptotriazine (200 mg) and the resulting mixture was stirred at room temperature for 3 hr. The mixture was filtered. The filtrate was concentrated to under reduced pressure give a crude product. The crude was diluted with a mixture of MTBE and n-Hexane (15 mL, 1:1) and stirred for 1 hr. The insoluble solid was filtered off, washed with MTBE—n-Hexane (5 mL, 1:1) and dried on air to afford 2-(4-cyclopropyl-6-methoxy-pyrimidin-5-yl)-9-[[4-[5-methoxy-3-(trifluoromethyl)pyrazol-1-yl]phenyl]methyl]purin-8-amine (270 mg, 502 μmol, 38.7% yield) as an off-white solid which was used in the next steps without further purification.

LCMS(ESI): [M+H]+ m/z: calcd 538.20; found 538.2.

¹H NMR (400 MHz, DMSO-d6) δ 0.75-0.82 (m, 2H), 0.94-1.02 (m, 2H), 1.60-1.66 (m, 1H), 3.81 (s, 3H), 3.96 (s, 3H), 5.36 (s, 2H), 6.44 (s, 1H), 7.38-7.46 (m, 4H), 7.58 (d, 2H), 8.53 (s, 1H), 8.62 (s, 1H).

### Step 5: The synthesis of 2-(4-cyclopropyl-6-methoxy-pyrimidin-5-yl)-9-[[4-[5-methoxy-3-(trifluoromethyl)pyrazol-1-yl]phenyl]methyl]-7-(2,2,2-trifluoroethyl)purin-8-imine (T-117) and 2-(4-cyclopropyl-6-methoxy-pyrimidin-5-yl)-9-[[4-[5-methoxy-3-(trifluoromethyl)pyrazol-1-yl]phenyl]methyl]-N-(2,2,2-trifluoroethyl)-7H-purin-8-imine

To a solution of 2-(4-cyclopropyl-6-methoxy-pyrimidin-5-yl)-9-[[4-[5-methoxy-3-(trifluoromethyl)pyrazol-1-yl]phenyl]methyl]purin-8-amine (130 mg, 242 μmol) in ACN (15.0 mL) was added cesium carbonate (158 mg, 484 μmol). The reaction mixture was stirred at room temperature for 5 minutes then 2,2,2-trifluoroethyl trifluoromethanesulfonate (84.2 mg, 363 mol, 52.3 μL) was added. The reaction mixture was stirred at 90° C. for 15 hr. The reaction mixture was cooled to room temperature and poured into water (10 mL). The obtained mixture was extracted with EtOAc (30 mL). The organic layer was separated, dried over anhydrous sodium sulfate and concentrated under reduced pressure. The residue was subjected to HPLC (gradient elution: 10-50% water+FA (0.1% vol.)—ACN+FA (0.1% vol.); flow: 30 mL/min, column: Waters SunFire C18, 100×19 mm, 5 μm) to afford 2-(4-cyclopropyl-6-methoxy-pyrimidin-5-yl)-9-[[4-[5-methoxy-3-(trifluoromethyl)pyrazol-1-yl]phenyl]methyl]-N-(2,2,2-trifluoroethyl)-7H-purin-8-imine (21.0 mg, 33.9 μmol, 14.0% yield) as an yellow solid and 2-(4-cyclopropyl-6-methoxy-pyrimidin-5-yl)-9-[[4-[5-methoxy-3-(trifluoromethyl)pyrazol-1-yl]phenyl]methyl]-7-(2,2,2-trifluoroethyl)purin-8-imine (25.0 mg, 70% purity by LCMS) which was re-purified by HPLC (gradient elution: 40-65% water+FA (0.1% vol.)—ACN+FA (0.1% vol.); flow: 30 mL/min, column: Waters SunFire C18, 100×19 mm, 5 μm) to afford 2-(4-cyclopropyl-6-methoxy-pyrimidin-5-yl)-9-[[4-[5-methoxy-3-(trifluoromethyl)pyrazol-1-yl]phenyl]methyl]-7-(2,2,2-trifluoroethyl)purin-8-imine (10.8 mg, 17.4 μmol, 7.20% yield) as an off-white powder. [1146] 2-(4-cyclopropyl-6-methoxy-pyrimidin-5-yl)-9-[[4-[5-methoxy-3-(trifluoromethyl)pyrazol-1-yl]phenyl]methyl]-7-(2,2,2-trifluoroethyl)purin-8-imine (T-117):

¹H NMR (600 MHz, DMSO-d6) δ 0.79-0.85 (m, 2H), 0.96-1.01 (m, 2H), 1.62-1.70 (m, 1H), 3.81 (s, 3H), 3.95 (s, 3H), 4.76-5.01 (m, 2H), 5.07-5.22 (m, 2H), 6.43 (s, 1H), 7.06-7.11 (m, 1H), 7.49 (d, 2H), 7.54-7.60 (m, 2H), 8.29-8.36 (m, 1H), 8.62 (s, 1H).

LCMS(ESI): [M+H]+ m/z: calcd 620.21; found 620.2. [1147] 2-(4-cyclopropyl-6-methoxy-pyrimidin-5-yl)-9-[[4-[5-methoxy-3-(trifluoromethyl)pyrazol-1-yl]phenyl]methyl]-N-(2,2,2-trifluoroethyl)-7H-purin-8-imine:

¹H NMR (600 MHz, DMSO-d6) δ 0.74-0.81 (m, 2H), 0.96-1.01 (m, 2H), 1.57-1.63 (m, 1H), 3.80 (s, 3H), 3.94 (s, 3H), 4.29-4.37 (m, 2H), 5.43 (s, 2H), 6.44 (s, 1H), 7.38 (d, 2H), 7.58 (d, 2H), 8.39 (t, 1H), 8.62 (s, 1H), 8.70 (s, 1H).

LCMS(ESI): [M+H]+ m/z: calcd 620.21; found 620.2.

### Example T-113

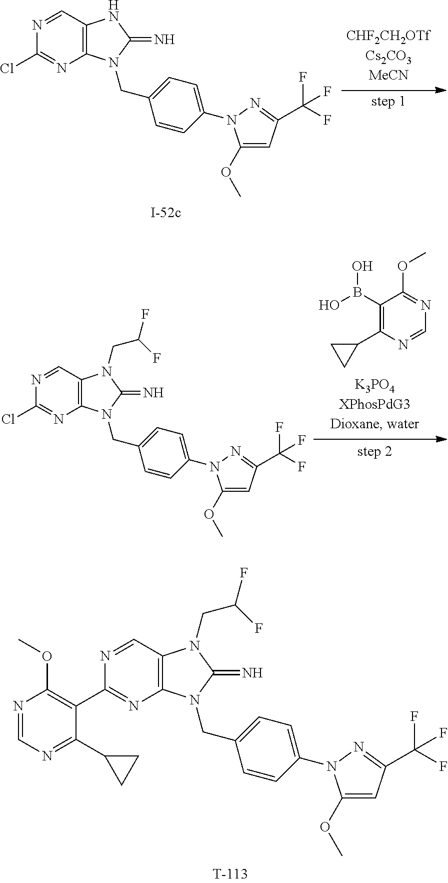 [SMILES: COc1cc(C(F)(F)F)nn1-c1ccc(Cn2c(=N)[nH]c3cnc(Cl)nc32)cc1 ; COc1cc(C(F)(F)F)nn1-c1ccc(Cn2c(=N)n(CC(F)F)c3cnc(Cl)nc32)cc1 ; COc1ncnc(C2CC2)c1B(O)O ; COc1ncnc(C2CC2)c1-c1ncc2c(n1)n(Cc1ccc(-n3nc(C(F)(F)F)cc3OC)cc1)c(=N)n2CC(F)F]

### Step 1: The synthesis of 2-chloro-7-(2,2-difluoroethyl)-9-[[4-[5-methoxy-3-(trifluoromethyl)pyrazol-1-yl]phenyl]methyl]purin-8-imine

The synthesis of the starting 2-chloro-9-[[4-[5-methoxy-3-(trifluoromethyl)pyrazol-1-yl]phenyl]methyl]-7H-purin-8-imine is described by Intermediate 52.

To a mixture of 2-chloro-9-[[4-[5-methoxy-3-(trifluoromethyl)pyrazol-1-yl]phenyl]methyl]-7H-purin-8-imine (0.50 g, 1.18 mmol) and cesium carbonate (577 mg, 1.77 mmol) in ACN (16 mL) 2,2-difluoroethyl trifluoromethanesulfonate (278 mg, 1.30 mmol) was added one portion. The resulting mixture was stirred at 50° C. for 16 hr. The reaction mixture was cooled to room temperature and diluted with water (5 mL). The resulting mixture was extracted with ethyl acetate (2×10 mL). The combined organic layers were washed with water (2×10 mL) and brine (10 mL) and concentrated under reduced pressure to afford 2-chloro-7-(2,2-difluoroethyl)-9-[[4-[5-methoxy-3-(trifluoromethyl)pyrazol-1-yl]phenyl]methyl]purin-8-imine (0.45 g, 922 μmol, 78.0% yield) as a yellow solid which was used in the next steps without further purification.

LCMS(ESI): [M+H]+ m/z: calcd 488.12; found 488.2.

### Step 2: Synthesis of 2-(4-cyclopropyl-6-methoxy-pyrimidin-5-yl)-7-(2,2-difluoroethyl)-9-[[4-[5-methoxy-3-(trifluoromethyl)pyrazol-1-yl]phenyl]methyl]purin-8-imine

2-Chloro-7-(2,2-difluoroethyl)-9-[[4-[5-methoxy-3-(trifluoromethyl)pyrazol-1-yl]phenyl]methyl]purin-8-imine (0.45 g, 922 μmol), (4-cyclopropyl-6-methoxy-pyrimidin-5-yl)boronic acid (358 mg, 1.84 mmol), potassium phosphate tribasic anhydrous (587 mg, 2.77 mmol) and XPhos Pd G3 (10.0 mg, 15.8 μmol) were mixed in a degassed mixture of dioxane (5 mL) and water (0.5 mL). The reaction mixture was stirred at 90° C. for 12 hr. The reaction mixture was cooled to room temperature and diluted with EtOAc (20 mL). The resulting mixture was washed with water (10 mL) and brine (10 mL). To the obtained organic phase SiliaMetS® Dimercaptotriazine (20 mg) was added, and the mixture was stirred for 30 min. The mixture was filtered and the filtrate was concentrated under reduced pressure. The residue was subjected to HPLC (gradient elution: 2-10 min, 10-50% water—methanol, +0.1% vol. of 25% aq. NH3, flow: 30 mL/min, column: SunFire C18 100×19 mm, 5 μm) to afford a product with 60% purity by LCMS which was repurified by HPLC (2-10 min, 30-80% water—acetonitrile, flow: 30 mL/min, column: SunFire C18 100×19 mm, 5 μm) to afford 2-(4-cyclopropyl-6-methoxy-pyrimidin-5-yl)-7-(2,2-difluoroethyl)-9-[[4-[5-methoxy-3-(trifluoromethyl)pyrazol-1-yl]phenyl]methyl]purin-8-imine (21.0 mg, 34.9 μmol, 3.78% yield) as a light-yellow solid.

¹H NMR (600 MHz, DMSO-d6) δ δ 0.79-0.82 (m, 2H), 0.97-1.01 (m, 2H), 1.63-1.69 (m, 1H), 3.81 (s, 3H), 3.95 (s, 3H), 4.32-4.50 (m, 2H), 5.02-5.24 (m, 2H), 6.38 (t, 1H, CHF2), 6.43 (s, 1H), 6.91 (br., 1H), 7.48 (d, 2H), 7.56 (d, 2H), 8.22-8.32 (m, 1H), 8.62 (s, 1H).

LCMS(ESI): [M+H]+ m/z: calcd 602.23; found 602.0.

### Example T-146

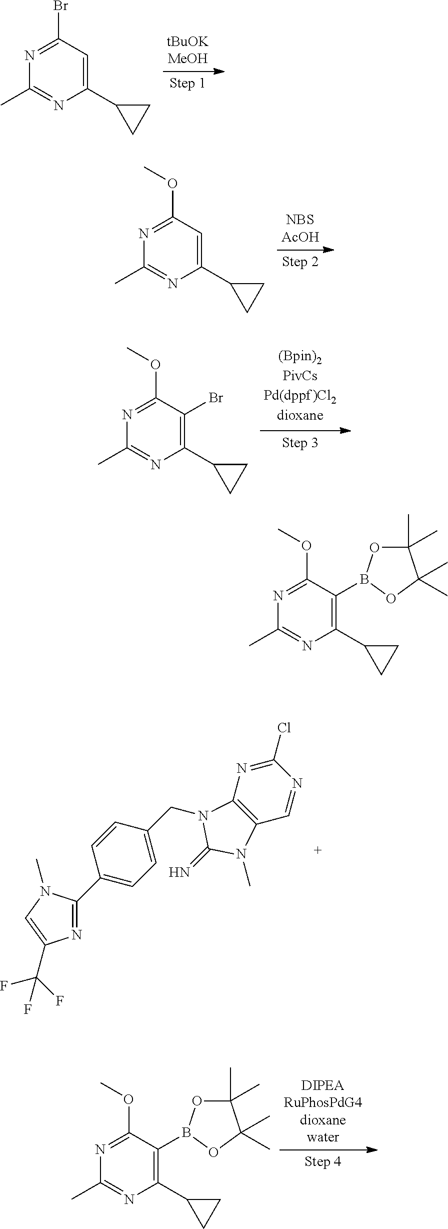

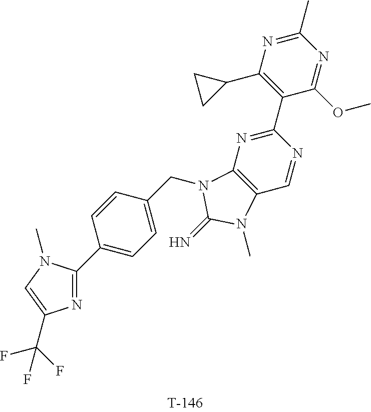 [SMILES: COc1nc(C)nc(C2CC2)c1-c1ncc2c(n1)n(Cc1ccc(-c3nc(C(F)(F)F)cn3C)cc1)c(=N)n2C]

### Step 1: The synthesis of 4-cyclopropyl-6-methoxy-2-methyl-pyrimidine

Potassium tert-butoxide (1.22 g, 10.8 mmol) was added portionwise to a solution of 4-bromo-6-cyclopropyl-2-methyl-pyrimidine (2.20 g, 10.3 mmol) in MeOH (25 mL) at 0° C. The reaction mixture was allowed to warm to room temperature and stirred at room temperature for 12 hr. The reaction mixture was concentrated under reduced pressure. The residue was diluted with DCM (200 mL) and washed with water (2×100 mL) and brine (150 ml). The organic layer was dried over anhydrous sodium sulfate and filtered. The filtrate was concentrated under reduced pressure to afford 4-cyclopropyl-6-methoxy-2-methyl-pyrimidine (1.30 g, 7.92 mmol, 76.7% yield) as a yellow liquid which was used in the next steps without further purification.

¹H NMR (400 MHz, CDCl3) δ 0.96-1.06 (m, 4H), 1.86-1.93 (m, 1H), 2.53 (s, 3H), 3.92 (s, 3H), 6.29 (s, 1H).

LCMS(ESI): [M+H]+ m/z: calcd 165.12; found 165.0.

### Step 2: The synthesis of 5-bromo-4-cyclopropyl-6-methoxy-2-methyl-pyrimidine

N-Bromosuccinimide (1.41 g, 7.92 mmol) was added to a stirred solution of 4-cyclopropyl-6-methoxy-2-methyl-pyrimidine (1.30 g, 7.92 mmol) in acetic acid (15.1 mL) at room temperature. The reaction mixture was stirred at 60° C. for 12 hr. The reaction mixture was cooled to room temperature and concentrated under reduced pressure. The residue was subjected to flash column chromatography chromatography (SiO2, gradient hexane—chloroform) to afford 5-bromo-4-cyclopropyl-6-methoxy-2-methyl-pyrimidine (1.00 g, 4.11 mmol, 52.0% yield) as a white solid.

¹H NMR (500 MHz, CDCl3) δ 0.99-1.05 (m, 2H), 1.11-1.16 (m, 2H), 2.43-2.50 (m, 4H), 4.00 (s, 3H).

LCMS(ESI): [M+H]+ m/z: calcd 243.02; found 243.0.

### Step 3: The synthesis of 4-cyclopropyl-6-methoxy-2-methyl-5-(4,4,5,5-tetramethyl-1,3,2-dioxaborolan-2-yl)pyrimidine

5-bromo-4-cyclopropyl-6-methoxy-2-methyl-pyrimidine (500 mg, 2.06 mmol), bis(pinacolato)diboron (575 mg, 2.26 mmol), cesium pivalate (963 mg, 4.11 mmol) and bis(diphenylphosphino)ferrocene]dichloropalladium(II)-DCM (168 mg, 206 μmol) were mixed in degassed dioxane (20 mL). The reaction mixture was stirred at 90° C. for 12 hr. The reaction mixture was cooled to room temperature and filtered. The filtrate was concentrated under reduced pressure to afford 4-cyclopropyl-6-methoxy-2-methyl-5-(4,4,5,5-tetramethyl-1,3,2-dioxaborolan-2-yl)pyrimidine (600 mg, crude) as a brown solid which was used in the next steps without further purification.

LCMS(ESI): [M+H]+ m/z: calcd 291.19; found 291.2.

### Step 4: The synthesis of 2-(4-cyclopropyl-6-methoxy-2-methyl-pyrimidin-5-yl)-7-methyl-9-[[4-[1-methyl-4-(trifluoromethyl)imidazol-2-yl]phenyl]methyl]purin-8-imine

2-chloro-7-methyl-9-[[4-[1-methyl-4-(trifluoromethyl)imidazol-2-yl]phenyl]methyl]purin-8-imine (189 mg, 449 μmol), 4-cyclopropyl-6-methoxy-2-methyl-5-(4,4,5,5-tetramethyl-1,3,2-dioxaborolan-2-yl)pyrimidine (300 mg, 1.03 mmol), RuPhosPdG4 (87.9 mg, 103 μmol) and DIPEA (267 mg, 2.07 mmol, 360 μL) were mixed in degassed mixture of dioxane (5.0 mL) and water (209 μL) under argon atmosphere at room temperature. The reaction mixture was stirred at 100° C. for 12 hr. The reaction mixture was cooled to room temperature and filtered. The filtrate was concentrated under reduced pressure. The residue was subjected to HPLC (0-5 min., 30-80% water—MeOH, +0.1% vol. of 25% aq. NH3, flow: 30 mL/min, column: XBridge C18 100×19 mm, 5 μm) to afford 2-(4-cyclopropyl-6-methoxy-2-methyl-pyrimidin-5-yl)-7-methyl-9-[[4-[1-methyl-4-(trifluoromethyl)imidazol-2-yl]phenyl]methyl]purin-8-imine (11.0 mg, 20.0 μmol, 1.94% yield) as a brown solid.

¹H NMR (600 MHz, DMSO-d6) δ 0.73-0.80 (m, 2H), 0.93-1.00 (m, 2H), 1.62-1.68 (m, 1H), 2.45 (s, 3H), 3.33-3.39 (m, 3H), 3.72 (s, 3H), 3.78 (s, 3H), 5.06-5.20 (m, 2H), 6.48-6.61 (m, 1H), 7.48 (d, 2H), 7.61-7.66 (m, 2H), 7.90 (s, 1H), 8.16-8.24 (m, 1H).

LCMS(ESI): [M+H]+ m/z: calcd 550.26; found 550.2.

### Example T-098

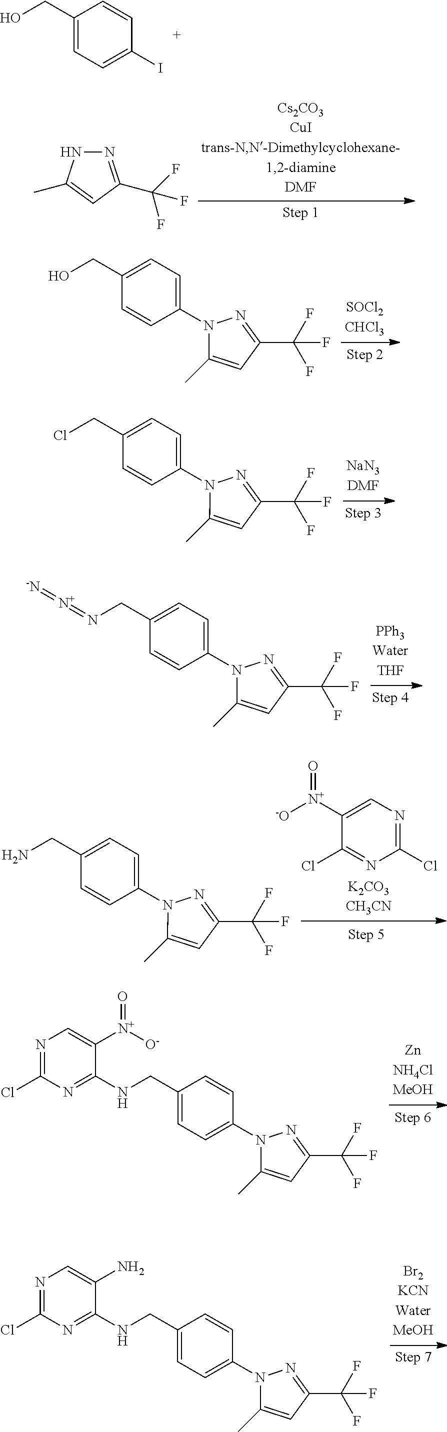

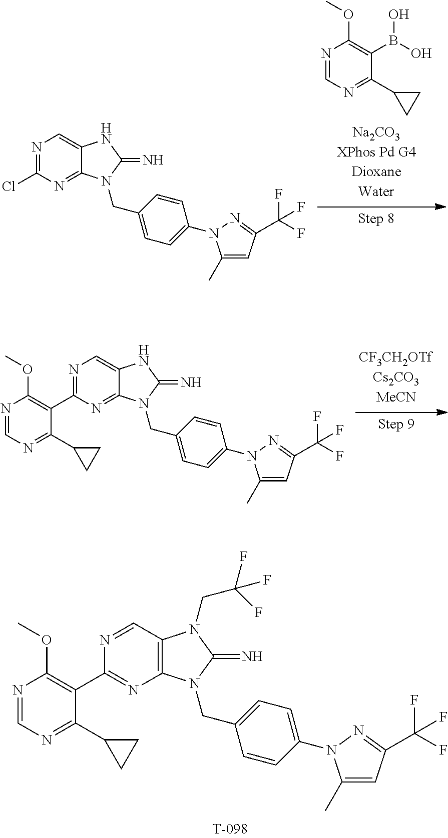 [SMILES: COc1ncnc(C2CC2)c1B(O)O ; Cc1cc(C(F)(F)F)nn1-c1ccc(Cn2c(=N)[nH]c3cnc(Cl)nc32)cc1 ; COc1ncnc(C2CC2)c1-c1ncc2[nH]c(=N)n(Cc3ccc(-n4nc(C(F)(F)F)cc4C)cc3)c2n1 ; COc1ncnc(C2CC2)c1-c1ncc2c(n1)n(Cc1ccc(-n3nc(C(F)(F)F)cc3C)cc1)c(=N)n2CC(F)(F)F]

### Step 1: The synthesis of [4-[5-methyl-3-(trifluoromethyl)pyrazol-1-yl]phenyl]methanol

A mixture of (4-iodophenyl)methanol (7.22 g, 30.8 mmol), 5-methyl-3-(trifluoromethyl)-1H-pyrazole (5.00 g, 33.3 mmol), cesium carbonate (21.1 g, 64.8 mmol), copper (I) iodide (705 mg, 3.70 mmol) and trans-N,N′-Dimethylcyclohexane-1,2-diamine (2.41 g, 16.9 mmol) in DMF (40.0 mL) was stirred at 110° C. in an inert atmosphere for 16 hr. The obtained mixture was cooled to room temperature and poured into water (80 mL). The obtained mixture was extracted with ethyl acetate (2×50 mL). The combined organic extracts were washed with brine, dried over anhydrous sodium sulfate, filtered and concentrated under reduced pressure. The residue (10.0 g) was subjected to flash-column chromatography (SiO2; gradient elution: 0-100% ethyl acetate in hexane) to afford [4-[5-methyl-3-(trifluoromethyl)pyrazol-1-yl]phenyl]methanol (1.90 g, 7.42 mmol, 24.0% yield) as light-yellow oil.

¹H NMR (400 MHz, CDCl3) δ 2.32 (s, 3H), 4.75 (s, 2H), 6.44 (s, 1H), 7.42 (d, 2H), 7.46 (d, 2H).

### Step 2: The synthesis of 1-[4-(chloromethyl)phenyl]-5-methyl-3-(trifluoromethyl)pyrazole

To a stirred solution of [4-[5-methyl-3-(trifluoromethyl)pyrazol-1-yl]phenyl]methanol (1.90 g, 7.42 mmol) in CHCl3 (50.0 mL) was added thionyl chloride (4.72 g, 39.6 mmol, 2.89 mL) dropwise at room temperature. The reaction mixture was heated at 50° C. for 1 hr. The resulting mixture was cooled to room temperature and concentrated under reduced pressure. The residue was quenched with saturated aqueous NaHCO₃ solution (50 mL) and extracted with DCM (3×35 mL). The combined organic layers were washed with water (40 mL), brine (40 mL), dried over anhydrous sodium sulfate, filtered and concentrated under reduced pressure to afford 1-[4-(chloromethyl)phenyl]-5-methyl-3-(trifluoromethyl)pyrazole (1.60 g, 5.83 mmol, 78.4% yield) as a light-brown solid which was used in the next steps without further purification.

¹H NMR (500 MHz, CDCl3) δ 2.37 (s, 3H), 4.64 (s, 2H), 6.47 (s, 1H), 7.46 (d, 2H), 7.52 (d, 2H).

LCMS(ESI): [M+H]+ m/z: calcd 275.07; found 275.0.

### Step 3: The synthesis of 1-[4-(azidomethyl)phenyl]-5-methyl-3-(trifluoromethyl)pyrazole

To a stirred solution of 1-[4-(chloromethyl)phenyl]-5-methyl-3-(trifluoromethyl)pyrazole (1.85 g, 6.74 mmol) in DMF (20.0 mL) was added sodium azide (876 mg, 13.5 mmol) at room temperature. The reaction mixture was heated at 80° C. for 16 hr. The obtained mixture was cooled to room temperature, poured into water (40 mL) and extracted with MTBE (3×50 mL). The combined organic layers were washed with water (50 mL), brine (50 mL), dried over anhydrous sodium sulfate, filtered and concentrated under reduced pressure to afford 1-[4-(azidomethyl)phenyl]-5-methyl-3-(trifluoromethyl)pyrazole (1.84 g, 6.54 mmol, 97.4% yield) as light-yellow oil which was used in the next steps without further purification.

¹H NMR (400 MHz, CDCl3) δ 2.35 (s, 3H), 4.41 (s, 2H), 6.45 (s, 1H), 7.42-7.49 (m, 4H).

LCMS(ESI): [M+H]+ m/z: calcd 282.1; found 282.2.

### Step 4: The synthesis of [4-[5-methyl-3-(trifluoromethyl)pyrazol-1-yl]phenyl]methanamine

To a stirred solution of 1-[4-(azidomethyl)phenyl]-5-methyl-3-(trifluoromethyl)pyrazole (1.84 g, 6.54 mmol) in THE (80 mL) and water (589 mg, 32.7 mmol, 589 μL) was added triphenylphosphine (2.06 g, 7.85 mmol) at room temperature. The reaction mixture was heated at 60° C. for 16 hr. The obtained mixture was cooled to room temperature and acidified with HCl (2 mL, 15% wt. in water). The resulting mixture was stirred for additional 1 hour at room temperature. The obtained mixture was concentrated under reduced pressure. The residue was triturated with toluene and the solid precipitate formed was filtered off, washed with toluene and n-hexane, and dried under reduced pressure to afford [4-[5-methyl-3-(trifluoromethyl)pyrazol-1-yl]phenyl]methanamine (1.70 g, 5.83 mmol, HCl salt, 89.0% yield) as a white solid which was used in the next steps without further purification.

¹H NMR (500 MHz, DMSO-d6) δ 2.35 (s, 3H), 4.12 (s, 2H), 6.78 (s, 1H), 7.64 (d, 2H), 7.71 (d, 2H), 8.64 (br, 3H).

LCMS(ESI): [M+H]+ m/z: calcd 256.12; found 256.0.

### Step 5: The synthesis of 2-chloro-N-[[4-[5-methyl-3-(trifluoromethyl)pyrazol-1-yl]phenyl]methyl]-5-nitro-pyrimidin-4-amine

To a stirred suspension of [4-[5-methyl-3-(trifluoromethyl)pyrazol-1-yl]phenyl]methanamine (1.00 g, 3.43 mmol, HCl salt) and 2,4-dichloro-5-nitro-pyrimidine (665 mg, 3.43 mmol) in CH3CN (19.6 mL) was added potassium carbonate (1.18 g, 8.57 mmol) at room temperature. The resulting reaction mixture was stirred for 16 hr at room temperature. The mixture was filtered and the filtrate was concentrated under reduced pressure to afford 2-chloro-N-[[4-[5-methyl-3-(trifluoromethyl)pyrazol-1-yl]phenyl]methyl]-5-nitro-pyrimidin-4-amine (1.44 g, 3.43 mmol, 100.0% yield) as a yellow gum which was used in the next steps without further purification.

¹H NMR (400 MHz, CDCl3) δ 2.35 (s, 3H), 4.89 (d, 2H), 6.44 (s, 1H), 7.40-7.51 (m, 4H), 8.71 (br, 1H), 9.06 (s, 1H).

LCMS(ESI): [M+H]+ m/z: calcd 413.07; found 413.0.

### Step 6: The synthesis of 2-chloro-N4-[[4-[5-methyl-3-(trifluoromethyl)pyrazol-1-yl]phenyl]methyl]pyrimidine-4,5-diamine

To a solution of 2-chloro-N-[[4-[5-methyl-3-(trifluoromethyl)pyrazol-1-yl]phenyl]methyl]-5-nitro-pyrimidin-4-amine (1.20 g, 2.91 mmol) in MeOH (100 mL) was added ammonium chloride (1.56 g, 29.1 mmol), followed by portionwise addition of zinc powder (950 mg, 14.5 mmol). The resulting reaction mixture was stirred at room temperature for 16 hr. The mixture was filtered and the filtrate was concentrated under reduced pressure. The residue was dissolved in ethyl acetate (150 mL) and washed with brine (50 mL), dried over anhydrous sodium sulfate and concentrated under reduced pressure to afford 2-chloro-N4-[[4-[5-methyl-3-(trifluoromethyl)pyrazol-1-yl]phenyl]methyl]pyrimidine-4,5-diamine (1.10 g, 2.87 mmol, 99.1% yield) as a red solid.

LCMS(ESI): [M+H]+ m/z: calcd 383.11; found 383.0.

### Step 7: The synthesis of -chloro-9-[[4-[5-methyl-3-(trifluoromethyl)pyrazol-1-yl]phenyl]methyl]-7H-purin-8-imine

To a stirred solution of Br2 (2.07 g, 12.9 mmol) in water (4.00 mL) was added a solution of potassium cyanide (842 mg, 12.9 mmol) in water (16.0 mL) dropwise at 0° C. The resulting mixture was stirred at 0° C. for 15 min. To the mixture was added a solution of 2-chloro-N4-[[4-[5-methyl-3-(trifluoromethyl)pyrazol-1-yl]phenyl]methyl]pyrimidine-4,5-diamine (1.10 g, 2.87 mmol) in methanol (60.0 mL) at 0° C. The reaction mixture was stirred at 50° C. for 16 hours. The obtained mixture was cooled to room temperature and concentrated under reduced pressure. The residue was dissolved in ethyl acetate (100 mL), washed with brine (50 mL), dried over anhydrous sodium sulfate, filtered and concentrated under reduced pressure to afford 2-chloro-9-[[4-[5-methyl-3-(trifluoromethyl)pyrazol-1-yl]phenyl]methyl]-7H-purin-8-imine (1.04 g, 2.55 mmol, 88.9% yield) as a red solid which was used in the next steps without further purification.

¹H NMR (400 MHz, CDCl3) δ 2.31 (s, 3H), 5.33 (s, 2H), 6.44 (s, 1H), 7.35 (d, 2H), 7.42 (d, 2H), 8.44 (s, 1H).

LCMS(ESI): [M+H]+ m/z: calcd 408.1; found 408.2.

### Step 8: The synthesis of 2-(4-cyclopropyl-6-methoxy-pyrimidin-5-yl)-9-[[4-[5-methyl-3-(trifluoromethyl)pyrazol-1-yl]phenyl]methyl]-7H-purin-8-imine

To a stirred solution of 2-chloro-9-[[4-[5-methyl-3-(trifluoromethyl)pyrazol-1-yl]phenyl]methyl]-7H-purin-8-imine (350 mg, 858 μmol) in a mixture of degassed dioxane (35.0 mL) and water (7.00 mL) was added (4-cyclopropyl-6-methoxy-pyrimidin-5-yl)boronic acid (167 mg, 858 μmol), sodium carbonate (273 mg, 2.57 mmol) and XPhos Pd G4 (36.9 mg, 42.9 μmol). The reaction mixture was stirred at 95° C. for 16 hours. The obtained mixture was cooled to room temperature and filtered through a pad of SiO2. The pad of SiO2 was washed with CH3CN (20 ml). The combined filtrate was concentrated under reduced pressure. The residue was dissolved in MeOH (30.0 mL) and SiliaMetS® Dimercaptotriazine (100 mg) was added. The mixture was stirred for 4 hours at room temperature, then filtered. The filtrate was concentrated under reduced pressure to afford 2-(4-cyclopropyl-6-methoxy-pyrimidin-5-yl)-9-[[4-[5-methyl-3-(trifluoromethyl)pyrazol-1-yl]phenyl]methyl]-7H-purin-8-imine (390 mg, crude) as a red solid which was used in the next steps without further purification.

LCMS(ESI): [M+H]+ m/z: calcd 522.21; found 522.2.

### Step 9: The synthesis of 2-(4-cyclopropyl-6-methoxy-pyrimidin-5-yl)-9-[[4-[5-methyl-3-(trifluoromethyl)pyrazol-1-yl]phenyl]methyl]-7-(2,2,2-trifluoroethyl)purin-8-imine

To a stirred solution of 2-(4-cyclopropyl-6-methoxy-pyrimidin-5-yl)-9-[[4-[5-methyl-3-(trifluoromethyl)pyrazol-1-yl]phenyl]methyl]-7H-purin-8-imine (390 mg, crude) in MeCN (50.0 mL) was added cesium carbonate (487 mg, 1.50 mmol) and 2,2,2-trifluoroethyl trifluoromethanesulfonate (208 mg, 897 μmol, 129 μL) at room temperature. The resulting reaction mixture was stirred at 70° C. for 16 hr. The mixture was cooled to room temperature, filtered through a pad of SiO2 and washed with CH3CN (20 mL). The combined filtrate was concentrated under reduced pressure. The residue was subjected to HPLC (gradient elution: 0-5 min, 35-60% water—ACN, +0.1% vol. of 25% aq. NH3, 30 mL/min, column: XBridge C18, 100×20 mm, 5 μm) to afford the impure product which was again subjected to HPLC (gradient elution: 0-5 min, 40-80% water+FA (0.1% vol.)—ACN+FA (0.1% vol.); flow: 30 mL/min, column: Chromatorex 18 SMB100-5T 100, 100×19 mm, 5 μm) to afford 2-(4-cyclopropyl-6-methoxy-pyrimidin-5-yl)-9-[[4-[5-methyl-3-(trifluoromethyl)pyrazol-1-yl]phenyl]methyl]-7-(2,2,2-trifluoroethyl)purin-8-imine (21.6 mg, 35.8 μmol, 4.79% yield) as an off-white solid.

¹H NMR (500 MHz, DMSO-d6) δ 0.79-0.84 (m, 2H), 0.97-1.01 (m, 2H), 1.64-1.70 (m, 1H), 2.29 (s, 3H), 3.81 (s, 3H), 4.78-5.03 (m, 2H), 5.08-5.29 (m, 2H), 6.73 (s, 1H), 7.02-7.18 (m, 1H), 7.52 (s, 4H), 8.33 (br, 1H), 8.62 (s, 1H).

LCMS(ESI): [M+H]+ m/z: calcd 604.22; found 604.2.

### Example T-116

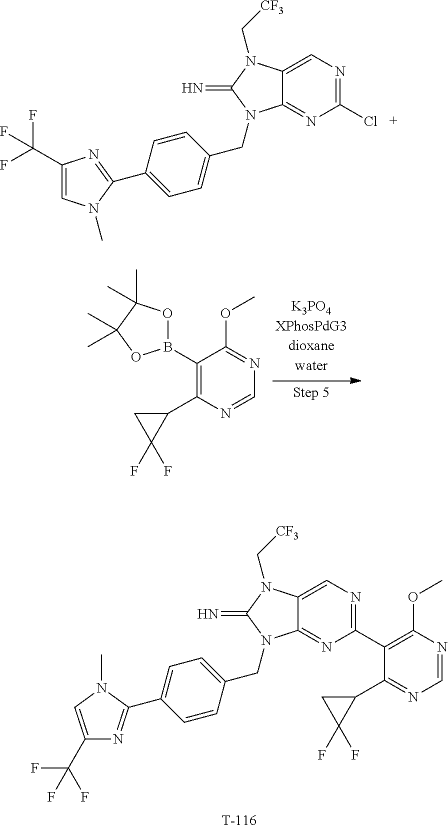

### Step 1: The synthesis of 2-[4-(2,2-difluorocyclopropyl)-6-methoxy-pyrimidin-5-yl]-9-[[4-[1-methyl-4-(trifluoromethyl)imidazol-2-yl]phenyl]methyl]-7-(2,2,2-trifluoroethyl)purin-8-imine

2-chloro-9-[[4-[1-methyl-4-(trifluoromethyl)imidazol-2-yl]phenyl]methyl]-7-(2,2,2-trifluoroethyl)purin-8-imine (200 mg, 408 μmol), 4-(2,2-difluorocyclopropyl)-6-methoxy-5-(4,4,5,5-tetramethyl-1,3,2-dioxaborolan-2-yl)pyrimidine (382 mg, 1.22 mmol), potassium phosphate tribasic anhydrous (260 mg, 1.22 mmol) and RuPhos Pd G4 (17.4 mg, 20.4 μmol) were mixed in degassed mixture of dioxane (6.0 mL) and water (1.0 mL). The reaction mixture was degassed. The reaction mixture was stirred at 70° C. for 16 hr. The reaction mixture was cooled to room temperature, diluted with EtOAc (25 mL) and washed with water (10 mL). The organic layer was separated, washed with brine (2×20 mL), dried over anhydrous sodium sulfate and filtered. SiliaMetS® Dimercaptotriazine (200 mg) was added to the filtrate and the resulting mixture was stirred for 1 hr. The resulting mixture was filtered. The filtrate was concentrated under reduced pressure. The residue was subjected to HPLC (gradient elution: 2-10 min., 0-60% ACN, flow: 30 mL/min, column: SunFire 100×19 mm, 5 μm) to afford 2-[4-(2,2-difluorocyclopropyl)-6-methoxy-pyrimidin-5-yl]-9-[[4-[1-methyl-4-(trifluoromethyl)imidazol-2-yl]phenyl]methyl]-7-(2,2,2-trifluoroethyl)purin-8-imine (7.50 mg, 11.7 μmol, 2.87% yield) as a light-yellow solid.

¹H NMR (600 MHz, DMSO-d6) δ 1.84-1.92 (m, 1H), 2.31-2.38 (m, 1H), 2.77-2.84 (m, 1H), 3.73 (s, 3H), 3.87 (s, 3H), 4.79-5.04 (m, 2H), 5.03-5.29 (m, 2H), 7.05-7.15 (m, 1H), 7.49 (d, 2H), 7.63-7.67 (m, 2H), 7.90 (s, 1H), 8.32-8.40 (m, 1H), 8.78 (s, 1H).

LCMS(ESI): [M+H]+ m/z: calcd 640.21; found 640.0.

### Example T-108

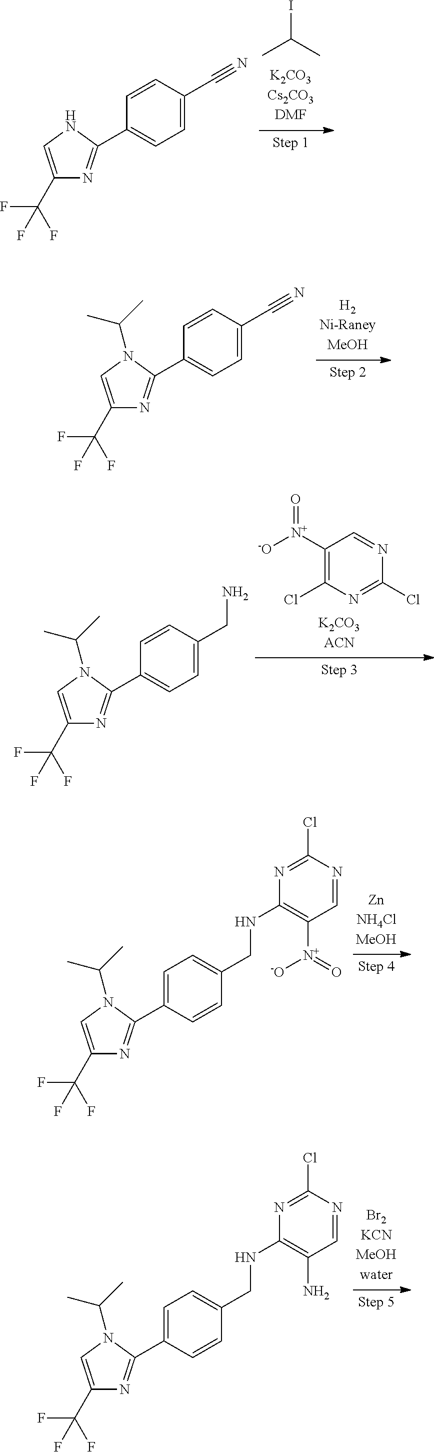

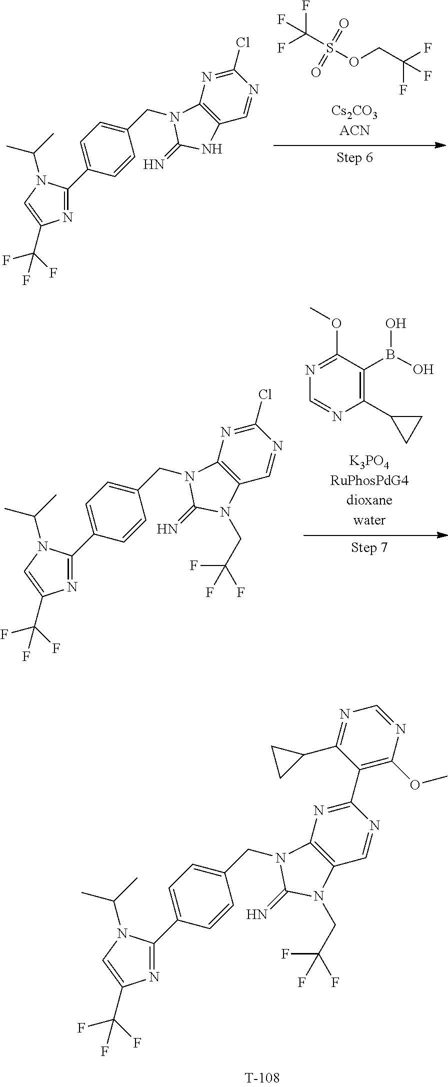

### Step 1: The synthesis of 4-[I-isopropyl-4-(trifluoromethyl)imidazol-2-yl]benzonitrile

Potassium carbonate (8.74 g, 63.2 mmol), cesium carbonate (4.12 g, 12.7 mmol) and 2-iodopropane (12.9 g, 75.9 mmol, 7.58 mL) were added to a solution of 4-[4-(trifluoromethyl)-1H-imidazol-2-yl]benzonitrile (6.00 g, 25.3 mmol) in DMF (100 mL). The reaction mixture was stirred at 90° C. for 72 hr. The reaction mixture was cooled to room temperature, poured into ice-cold water (200 mL) and extracted with EtOAc (100 mL). The organic layer was washed with brine (3×100 mL) and concentrated under reduced pressure to afford 4-[1-isopropyl-4-(trifluoromethyl)imidazol-2-yl]benzonitrile (5.00 g, 17.9 mmol, 70.8% yield) as a yellow solid which was used in the next step without further purification.

¹H NMR (500 MHz, DMSO-d6) δ 1.41 (d, 6H), 4.45-4.52 (m, 1H), 7.78 (d, 2H), 7.99 (d, 2H), 8.25 (s, 1H).

LCMS(ESI): [M+H]+ m/z: calcd 280.13; found 280.0.

### Step 2: The synthesis of [4-[I-isopropyl-4-(trifluoromethyl)imidazol-2-yl]phenyl]methanamine

A solution of 4-[1-isopropyl-4-(trifluoromethyl)imidazol-2-yl]benzonitrile (5.00 g, 17.9 mmol) and Ni-Raney (500 mg) in MeOH (400 mL) was subjected for hydrogenation at 40 atm for 12 hr. The reaction mixture was filtered through a thin pad of silica gel. The filtrate was concentrated under reduced pressure. The resulting residue was dissolved in DCM (100 mL), dried over anhydrous sodium sulfate, filtered and concentrated under reduced pressure to afford [4-[1-isopropyl-4-(trifluoromethyl)imidazol-2-yl]phenyl]methanamine (5.00 g, 17.7 mmol, 98.6% yield) as a brown solid which was used in the next step without further purification.

¹H NMR (400 MHz, DMSO-d6) δ 1.39 (d, 6H), 3.80 (s, 2H), 4.41-4.51 (m, 1H), 7.39-7.52 (m, 4H), 8.15 (s, 1H).

LCMS(ESI): [M+H]+ m/z: calcd 284.17; found 284.0.

### Step 3: The synthesis of 2-chloro-N-[[4-[1-isopropyl-4-(trifluoromethyl)imidazol-2-yl]phenyl]methyl]-5-nitro-pyrimidin-4-amine

2,4-Dichloro-5-nitro-pyrimidine (3.57 g, 18.4 mmol) and potassium carbonate (3.66 g, 26.5 mmol) were added to a solution of [4-[1-isopropyl-4-(trifluoromethyl)imidazol-2-yl]phenyl]methanamine (5.00 g, 17.7 mmol) in ACN (100 mL). The reaction mixture was stirred at room temperature for 18 hr. The reaction mixture was concentrated under reduced pressure. The residue was diluted with water (100 mL) and extracted with EtOAc (3×50 mL). The combined organic layers were dried over anhydrous sodium sulfate and concentrated under reduced pressure to afford 2-chloro-N-[[4-[1-isopropyl-4-(trifluoromethyl)imidazol-2-yl]phenyl]methyl]-5-nitro-pyrimidin-4-amine (7.00 g, 15.9 mmol, 90.0% yield) as a brown solid which was used in the next step without further purification.

LCMS(ESI): [M+H]+ m/z: calcd 441.12; found 441.2.

### Step 4: The synthesis of 2-chloro-N4-[[4-[1-isopropyl-4-(trifluoromethyl)imidazol-2-yl]phenyl]methyl]pyrimidine-4,5-diamine

Ammonium chloride (12.7 g, 238 mmol) and zinc (6.23 g, 95.3 mmol) were added to a solution of 2-chloro-N-[[4-[1-isopropyl-4-(trifluoromethyl)imidazol-2-yl]phenyl]methyl]-5-nitro-pyrimidin-4-amine (7.00 g, 15.9 mmol) in MeOH (200 mL) at 0° C. The reaction mixture was stirred at ambient temperature for 18 hr. The reaction mixture was filtered. The filtrate was concentrated under reduced pressure. The residue was diluted with water (200 mL) and extracted with EtOAc (3×100 mL). The combined organic layers were dried over anhydrous sodium sulfate and concentrated under reduced pressure to afford 2-chloro-N4-[[4-[1-isopropyl-4-(trifluoromethyl)imidazol-2-yl]phenyl]methyl]pyrimidine-4,5-diamine (6.00 g, 14.6 mmol, 92.0% yield) as a brown solid which was used in the next step without further purification.

LCMS(ESI): [M+H]+ m/z: calcd 411.16; found 411.2.

### Step 5: The synthesis of 2-chloro-9-[[4-[1-isopropyl-4-(trifluoromethyl)imidazol-2-yl]phenyl]methyl]-7H-purin-8-imine

A solution of potassium cyanide (6.75 g, 104 mmol) in water (20 mL) was added dropwise to a solution of Br2 (16.4 g, 103 mmol) in MeOH (200 mL) at 0° C. The resulting mixture was stirred at 0° C. for 15 min. A solution of 2-chloro-N4-[[4-[1-isopropyl-4-(trifluoromethyl)imidazol-2-yl]phenyl]methyl]pyrimidine-4,5-diamine (6.00 g, 14.6 mmol) in MeOH (20 mL) was added to the mixture. The reaction mixture was stirred at 0° C. for 18 hr. The reaction mixture was allowed to warm to room temperature, diluted with water (200 mL) and extracted with EtOAc (3×100 mL). The combined organic layers were dried over anhydrous sodium sulfate and concentrated under reduced pressure. The residue was subjected to column chromatography (SiO2, gradient elution: MTBE—methanol) to afford 2-chloro-9-[[4-[1-isopropyl-4-(trifluoromethyl)imidazol-2-yl]phenyl]methyl]-7H-purin-8-imine (3.80 g, 8.72 mmol, 59.7% yield) as a brown solid.

LCMS(ESI): [M+H]+ m/z: calcd 436.15; found 436.2.

### Step 6: The synthesis of 2-chloro-9-[[4-[1-isopropyl-4-(trifluoromethyl)imidazol-2-yl]phenyl]methyl]-7-(2,2,2-trifluoroethyl)purin-8-imine

Cesium carbonate (2.43 g, 7.46 mmol) followed by 2,2,2-trifluoroethyl trifluoromethanesulfonate (1.04 g, 4.47 mmol, 645 μL) were added to a solution of 2-chloro-9-[[4-[1-isopropyl-4-(trifluoromethyl)imidazol-2-yl]phenyl]methyl]-7H-purin-8-imine (1.30 g, 2.98 mmol) in ACN (60 mL). The reaction mixture was stirred at 80° C. for 96 hr. The reaction mixture was cooled to room temperature, diluted with water (100 mL) and extracted with EtOAc (3×50 mL). The combined organic layers were dried over anhydrous sodium sulfate and concentrated under reduced pressure. The residue was subjected to flash column chromatography (SiO2, gradient elution: 9:1 EtOAc:Hex to 100% EtOAc), then repurified by HPLC (gradient elution: 2-10 min, 0-55% water+FA (0.1% vol.)—ACN+FA (0.1% vol.); flow: 30 mL/min, column: SunFire C18, 100×19 mm, 5 μm) to afford 2-chloro-9-[[4-[1-isopropyl-4-(trifluoromethyl)imidazol-2-yl]phenyl]methyl]-7-(2,2,2-trifluoroethyl)purin-8-imine (100 mg, 193 μmol, 6.47% yield) as a brown solid.

LCMS(ESI): [M+H]+ m/z: calcd 518.16; found 518.0.

### Step 7: The synthesis of 2-(4-cyclopropyl-6-methoxy-pyrimidin-5-yl)-9-[[4-[I-isopropyl-4-(trifluoromethyl)imidazol-2-yl]phenyl]methyl]-7-(2,2,2-trifluoroethyl)purin-8-imine

2-Chloro-9-[[4-[1-isopropyl-4-(trifluoromethyl)imidazol-2-yl]phenyl]methyl]-7-(2,2,2-trifluoroethyl)purin-8-imine (40.0 mg, 77.2 μmol), (4-cyclopropyl-6-methoxy-pyrimidin-5-yl)boronic acid (30.0 mg, 155 μmol) and potassium phosphate tribasic (41.0 mg, 193 μmol) were mixed in a degassed mixture of dioxane (9.0 mL) and water (1.0 mL). RuPhos Pd G4 (6.57 mg, 7.72 μmol) was added to the mixture. The reaction mixture was stirred at 90° C. for 12 hr. The reaction mixture was cooled to room temperature and concentrated under reduced pressure. The resulting residue was diluted with water (10 mL) and extracted with MTBE (4×10 mL). The combined organic layers were dried over anhydrous sodium sulfate and concentrated under reduced pressure. The resulting residue was subjected to HPLC (gradient elution: 2-10 min., 0-55% water—ACN, +0.1% vol. of 25% aq. NH4OH, flow: 30 mL/min, column: SunFire 100×19 mm, 5 μm) to afford 2-(4-cyclopropyl-6-methoxy-pyrimidin-5-yl)-9-[[4-[1-isopropyl-4-(trifluoromethyl)imidazol-2-yl]phenyl]methyl]-7-(2,2,2-trifluoroethyl)purin-8-imine (10.0 mg, 15.8 μmol, 20.5% yield) as an off-white solid.

¹H NMR (600 MHz, DMSO-d6) δ 0.79-0.85 (m, 2H), 0.97-1.02 (m, 2H), 1.38 (d, 6H), 1.66-1.72 (m, 1H), 3.83 (s, 3H), 4.37-4.46 (m, 1H), 4.78-5.04 (m, 2H), 5.09-5.27 (m, 2H), 7.08-7.14 (m, 1H), 7.48-7.55 (m, 4H), 8.16 (s, 1H), 8.32-8.38 (m, 1H), 8.64 (s, 1H).

LCMS(ESI): [M+H]+ m/z: calcd 632.27; found 633.0.

### Example T-124

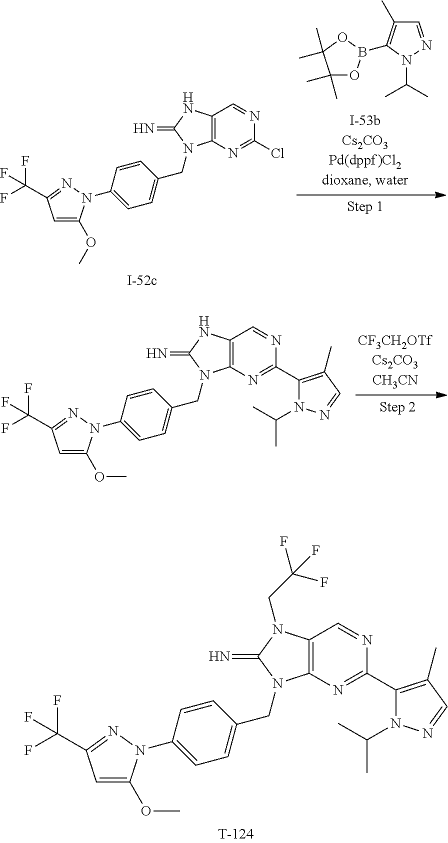 [SMILES: Cc1cnn(C(C)C)c1B1OC(C)(C)C(C)(C)O1 ; COc1cc(C(F)(F)F)nn1-c1ccc(Cn2c(=N)[nH]c3cnc(Cl)nc32)cc1 ; COc1cc(C(F)(F)F)nn1-c1ccc(Cn2c(=N)[nH]c3cnc(-c4c(C)cnn4C(C)C)nc32)cc1 ; COc1cc(C(F)(F)F)nn1-c1ccc(Cn2c(=N)n(CC(F)(F)F)c3cnc(-c4c(C)cnn4C(C)C)nc32)cc1]

The synthesis 2-chloro-9-[[4-[5-methoxy-3-(trifluoromethyl)pyrazol-1-yl]phenyl]methyl]-7H-purin-8-imine is described by Intermediate 52.

### Step 1: The synthesis of 2-(2-isopropyl-4-methyl-pyrazol-3-yl)-9-[[4-[5-methoxy-3-(trifluoromethyl)pyrazol-1-yl]phenyl]methyl]-7H-purin-8-imine

The synthesis of the starting 1-isopropyl-4-methyl-5-(4,4,5,5-tetramethyl-1,3,2-dioxaborolan-2-yl)pyrazole (I-53b) is described by Intermediate 53.

2-Chloro-9-[[4-[5-methoxy-3-(trifluoromethyl)pyrazol-1-yl]phenyl]methyl]-7H-purin-8-imine (200 mg, 472 μmol), 1-isopropyl-4-methyl-5-(4,4,5,5-tetramethyl-1,3,2-dioxaborolan-2-yl)pyrazole (200 mg, 802 μmol), cesium carbonate (461 mg, 1.42 mmol) and bis(diphenylphosphino)ferrocene]dichloropalladium(II)-DCM (38.0 mg, 47.2 μmol) were mixed in a degassed mixture of dioxane (15 mL) and water (3 mL) under an inert atmosphere. The resulting mixture was stirred at 80° C. for 18 hr. The reaction mixture was cooled to room temperature and concentrated under reduced pressure. The residue was diluted with water (20 mL) and EtOAc (50 mL). The organic layer was separated, washed with water (2×10 mL), filtered through a short pad of silica. The filtrate was concentrated under reduced pressure. The residue was subjected to HPLC (gradient elution: 2-10 min, 20-45% water—ACN, +0.1% vol. of 25% aq. NH4OH, flow: 30 mL/min, column: SunFire C18 100×19 mm, 5 μm) to afford 2-(2-isopropyl-4-methyl-pyrazol-3-yl)-9-[[4-[5-methoxy-3-(trifluoromethyl)pyrazol-1-yl]phenyl]methyl]-7H-purin-8-imine (35.0 mg, 68.4 μmol, 14.50% yield) as brown gum which was used in the next steps without further purification.

LCMS(ESI): [M+H]+ m/z: calcd 512.24; found 512.2.

### Step 2: The synthesis of 2-(2-isopropyl-4-methyl-pyrazol-3-yl)-9-[[4-[5-methoxy-3-(trifluoromethyl)pyrazol-1-yl]phenyl]methyl]-7-(2,2,2-trifluoroethyl)purin-8-imine

Cesium carbonate (55.0 mg, 171 μmol) was added to a stirred solution of 2-(2-isopropyl-4-methyl-pyrazol-3-yl)-9-[[4-[5-methoxy-3-(trifluoromethyl)pyrazol-1-yl]phenyl]methyl]-7H-purin-8-imine (35.0 mg, 68.4 μmol) in ACN (6 mL) at 70° C. The mixture was stirred for 5 min then 2,2,2-trifluoroethyl trifluoromethanesulfonate (31.0 mg, 137 μmol, 19.7 μL) was added to the reaction mixture. The mixture was stirred at 70° C. for 18 hr. The mixture was cooled to room temperature, diluted with water (10 mL) and extracted with EtOAc (2×20 mL). The combined organic layers were dried over anhydrous sodium sulfate and concentrated under reduced pressure. The residue was subjected to HPLC (gradient elution: 2-10 min, 20-45% water—ACN, +0.1% vol. of 25% aq. NH3, flow: 30 mL/min, column: SunFire C18 100×19 mm, 5 μm) to afford 2-(2-isopropyl-4-methyl-pyrazol-3-yl)-9-[[4-[5-methoxy-3-(trifluoromethyl)pyrazol-1-yl]phenyl]methyl]-7-(2,2,2-trifluoroethyl)purin-8-imine (4.70 mg, 7.92 μmol, 11.6% yield) as a yellow solid.

¹H NMR (400 MHz, DMSO-d6) δ 1.24-1.30 (m, 6H), 2.10 (s, 3H), 3.95 (s, 3H), 4.78-5.02 (m, 2H), 5.09-5.26 (m, 3H), 6.43 (s, 1H), 7.08-7.14 (m, 1H), 7.30 (s, 1H), 7.43-4.52 (m, 2H), 7.57-7.62 (m, 2H), 8.29-8.36 (m, 1H).

LCMS(ESI): [M+H]+ m/z: calcd 594.25; found 594.0.

### Example T-123

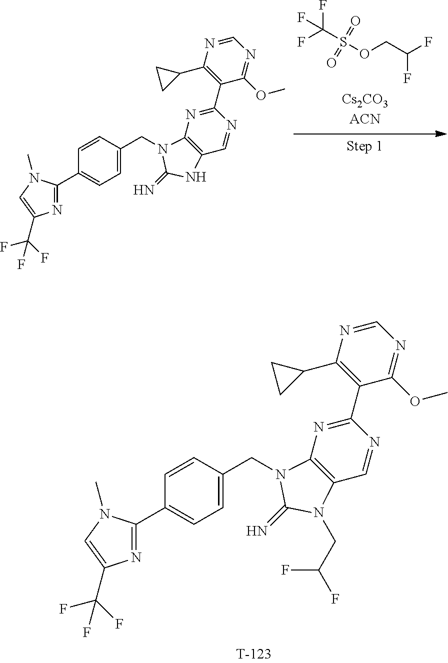 [SMILES: COc1ncnc(C2CC2)c1-c1ncc2[nH]c(=N)n(Cc3ccc(-c4nc(C(F)(F)F)cn4C)cc3)c2n1 ; O=S(=O)(OCC(F)F)C(F)(F)F ; COc1ncnc(C2CC2)c1-c1ncc2c(n1)n(Cc1ccc(-c3nc(C(F)(F)F)cn3C)cc1)c(=N)n2CC(F)F]

### Step 1: The synthesis of 2-(4-cyclopropyl-6-methoxy-pyrimidin-5-yl)-7-(2,2-difluoroethyl)-9-[[4-[1-methyl-4-(trifluoromethyl)imidazol-2-yl]phenyl]methyl]purin-8-imine

2,2-difluoroethyl trifluoromethanesulfonate (63.2 mg, 295 μmol) was added to a stirred suspension of 2-(4-cyclopropyl-6-methoxy-pyrimidin-5-yl)-9-[[4-[1-methyl-4-(trifluoromethyl)imidazol-2-yl]phenyl]methyl]-7H-purin-8-imine (140 mg, 269 μmol) and cesium carbonate (105 mg, 322 μmol) in ACN (5.0 mL). The reaction mixture was stirred at 80° C. for 24 hr. The reaction mixture was cooled to room temperature and concentrated under reduced pressure. The residue was diluted with water (10 mL) and extracted with EtOAc (20 mL). The organic layer was washed with brine (30 mL), dried over anhydrous sodium sulfate and concentrated under reduced pressure. The residue was subjected to HPLC (0-5 min., 10-60% water—ACN, +0.1% vol. of 25% aq. NH3, flow: 30 mL/min, column: XBridge C18 100×19 mm, 5 μm) to afford 2-(4-cyclopropyl-6-methoxy-pyrimidin-5-yl)-7-(2,2-difluoroethyl)-9-[[4-[1-methyl-4-(trifluoromethyl)imidazol-2-yl]phenyl]methyl]purin-8-imine (20.5 mg, 35.0 μmol, 13.0% yield) as a yellow solid.

¹H NMR (600 MHz, DMSO-d6) δ 0.79-0.84 (m, 2H), 0.97-1.01 (m, 2H), 1.65-1.70 (m, 1H), 3.73 (s, 3H), 3.82 (s, 3H), 4.31-4.50 (m, 2H), 5.07-5.24 (m, 2H), 6.26-6.50 (m, 1H, CHF2), 6.87-6.93 (m, 1H), 7.48 (d, 2H), 7.61-7.68 (m, 2H), 7.90 (s, 1H), 8.22-8.33 (m, 1H), 8.62 (s, 1H).

LCMS(ESI): [M+H]+ m/z: calcd 586.24; found 586.0.

### Example T-133

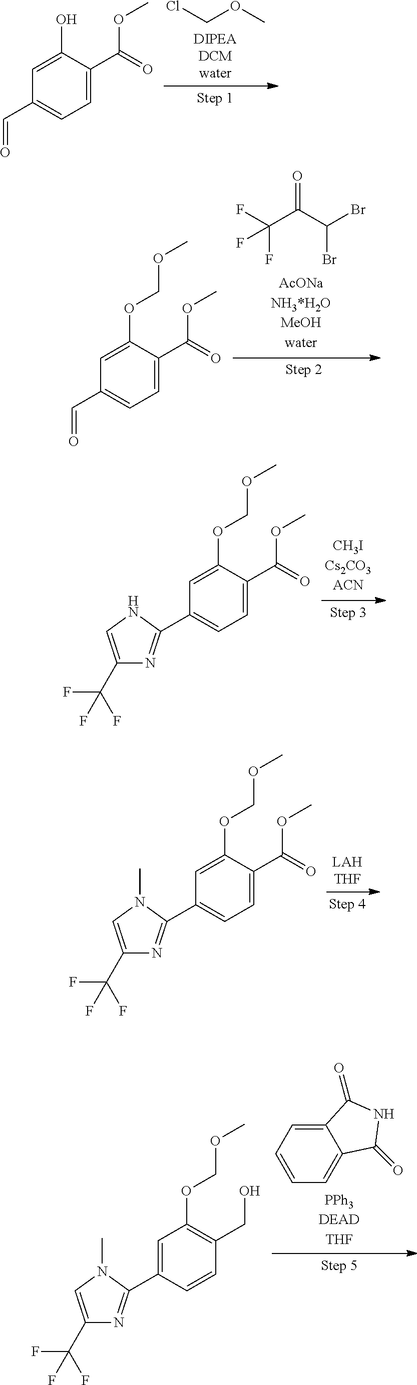

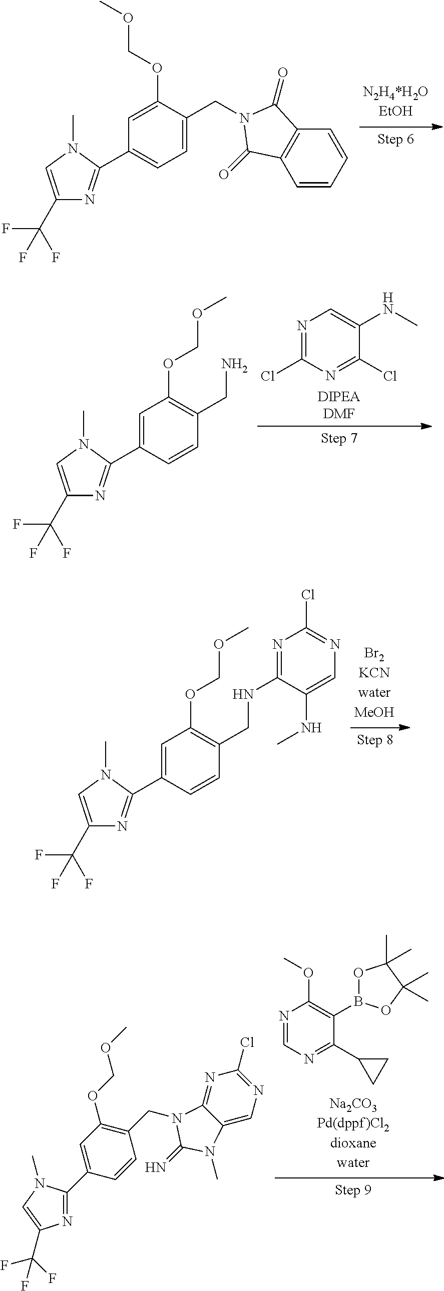

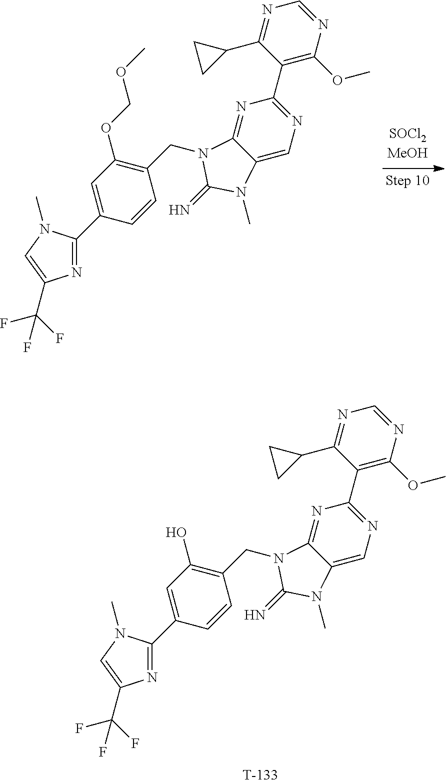 [SMILES: COCOc1cc(-c2nc(C(F)(F)F)cn2C)ccc1Cn1c(=N)n(C)c2cnc(-c3c(OC)ncnc3C3CC3)nc21 ; COc1ncnc(C2CC2)c1-c1ncc2c(n1)n(Cc1ccc(-c3nc(C(F)(F)F)cn3C)cc1O)c(=N)n2C]

### Step 1: The synthesis of methyl 4-formyl-2-(methoxymethoxy)benzoate

DIPEA (9.30 g, 71.9 mmol, 12.5 mL) was added to a stirred solution of methyl 4-formyl-2-hydroxybenzoate (9.60 g, 53.3 mmol) in DCM (200 mL). Chloro(methoxy)methane (5.36 g, 66.6 mmol) was added dropwise to the mixture. The reaction mixture was stirred at room temperature for 12 hr. The organic layer was separated, dried over anhydrous sodium sulfate and filtered. The filtrate was concentrated under reduced pressure to afford methyl 4-formyl-2-(methoxymethoxy)benzoate (12.0 g, 53.3 mmol, 100% yield) as a yellow oil which was used in the next steps without further purification.

¹H NMR (400 MHz, CDCl3) δ 3.49 (s, 3H), 3.89 (s, 3H), 5.29 (s, 2H), 7.51 (d, 1H), 7.65 (s, 1H), 7.84 (d, 1H), 9.98 (s, 1H).

### Step 2: The synthesis of methyl 2-(methoxymethoxy)-4-(4-(trifluoromethyl)-1H-imidazol-2-yl)benzoate

Sodium acetate (1.11 g, 13.5 mmol) was added to a mixture of 3,3-dibromo-1,1,1-trifluoropropan-2-one (3.61 g, 13.4 mmol) in water (10 mL). The reaction mixture was stirred at 100° C. for 45 min. The reaction mixture was cooled to room temperature. A solution of methyl 4-formyl-2-(methoxymethoxy)benzoate (3.00 g, 13.4 mmol) and aqueous ammonium hydroxide (12 mL, 25% wt.) in methanol (60 mL) and a was added to the reaction mixture. The resulting mixture was stirred at room temperature for 45 min, then at 100° C. for 45 min. The reaction mixture was cooled to room temperature and concentrated under reduced pressure. The residue was diluted with MeOH (15 mL). The resulting mixture was stirred at 40° C. for 10 min. The mixture was cooled to room temperature and diluted with water (15 mL). The resulting mixture was stirred at room temperature for 20 min, then solids were filtered off and air dried to afford methyl 2-(methoxymethoxy)-4-(4-(trifluoromethyl)-1H-imidazol-2-yl)benzoate (2.40 g, 7.27 mmol, 54.3% yield) as a yellow solid which was used in the next steps without further purification.

LCMS(ESI): [M+H]+ m/z: calcd 331.10; found 331.2

### Step 3: The synthesis of methyl 2-(methoxymethoxy)-4-(1-methyl-4-(trifluoromethyl)-1H-imidazol-2-yl)benzoate

Cesium carbonate (4.74 g, 14.5 mmol) and a solution of iodomethane (1.13 g, 7.99 mmol, 498 μL) in ACN (1.0 mL) were added to a solution of methyl 2-(methoxymethoxy)-4-[5-(trifluoromethyl)-1H-imidazol-2-yl]benzoate (2.40 g, 7.27 mmol) in ACN (30 mL) sequentially. The reaction mixture was stirred at room temperature for 12 hr. The reaction mixture was concentrated under reduced pressure. The residue was diluted with water (50 mL) and extracted with DCM (100 mL). The organic layer was washed with brine (50 mL), dried over anhydrous sodium sulfate and concentrated under reduced pressure to afford methyl 2-(methoxymethoxy)-4-(1-methyl-4-(trifluoromethyl)-1H-imidazol-2-yl)benzoate (2.50 g, 7.26 mmol, 99.9% yield) as a yellow oil which was used in the next steps without further purification.

¹H NMR (400 MHz, DMSO-d6) δ 3.42 (s, 3H), 3.82 (s, 3H), 3.84 (s, 3H), 5.33 (s, 2H), 7.44 (d, 1H), 7.53 (s, 1H), 7.78 (d, 1H), 8.00 (s, 1H).

LCMS(ESI): [M+H]+ m/z: calcd 345.12; found 345.2.

### Step 4: The synthesis of (2-(methoxymethoxy)-4-(1-methyl-4-(trifluoromethyl)-1H-imidazol-2-yl)phenyl)methanol

A solution of methyl 2-(methoxymethoxy)-4-(1-methyl-4-(trifluoromethyl)-1H-imidazol-2-yl)benzoate (5.00 g, 14.5 mmol) in THE (20 mL) was added dropwise to vigorously stirred suspension of LAH (1.10 g, 29.1 mmol) in THE (160 mL) at −20° C. The reaction mixture was stirred at −20° C. for 1 hr. The reaction mixture was quenched by dropwise addition of water (1.1 mL) in THE (4.0 mL), followed by an aqueous solution of NaOH (1.0 mL, 15% wt.) and water (3.0 mL). The mixture was filtered. The filtrate was concentrated under reduced pressure to afford (2-(methoxymethoxy)-4-(1-methyl-4-(trifluoromethyl)-1H-imidazol-2-yl)phenyl)methanol (5.00 g, crude) as a yellow gum which was used in the next steps without further purification.

LCMS(ESI): [M+H]+ m/z: calcd 317.13; found 317.2.

### Step 5: The synthesis of 2-(2-(methoxymethoxy)-4-(1-methyl-4-(trifluoromethyl)-1H-imidazol-2-yl)benzyl)isoindoline-1,3-dione

Triphenylphosphine (5.39 g, 20.6 mmol), phthalimide (3.02 g, 20.6 mmol) and diethyl azodicarboxylate (3.58 g, 20.6 mmol) were added to a solution of [2-(methoxymethoxy)-4-[1-methyl-4-(trifluoromethyl)imidazol-2-yl]phenyl]methanol (5.00 g, crude) in THE (100 mL). The reaction mixture was stirred at room temperature for 12 hr. The reaction mixture was concentrated under reduced pressure. The residue was diluted with water (100 mL) and extracted with DCM (2×100 mL). The combined organic layers were dried over anhydrous sodium sulfate and filtered. The filtrate was concentrated under reduced pressure. The residue was subjected to flash-column chromatography (SiO2, gradient hexane—ethyl acetate) to afford 2-(2-(methoxymethoxy)-4-(1-methyl-4-(trifluoromethyl)-1H-imidazol-2-yl)benzyl)isoindoline-1,3-dione (2.50 g, 5.61 mmol, 38.7% yield from methyl 2-(methoxymethoxy)-4-(1-methyl-4-(trifluoromethyl)-1H-imidazol-2-yl)benzoate) as a white solid.

¹H NMR (500 MHz, DMSO-d6) δ 3.40 (s, 3H), 3.75 (s, 3H), 4.84 (s, 2H), 5.34 (s, 2H), 7.22-7.29 (m, 2H), 7.39 (s, 1H), 7.81-7.95 (m, 5H).

LCMS(ESI): [M+H]+ m/z: calcd 446.15; found 446.2.

### Step 6: The synthesis of (2-(methoxymethoxy)-4-(1-methyl-4-(trifluoromethyl)-1H-imidazol-2-yl)phenyl)methanamine

An aqueous hydrazine hydrate (10.1 mmol, 1.23 mL, 35% wt.) was added to a solution of 2-[[2-(methoxymethoxy)-4-[1-methyl-4-(trifluoromethyl)imidazol-2-yl]phenyl]methyl]isoindoline-1,3-dione (3.00 g, 6.74 mmol) in EtOH (50 mL). The reaction mixture was stirred at 78° C. for 1 hr. The reaction mixture was cooled to room temperature. The solids were filtered out. The filtrate was concentrated under reduced pressure. The residue was subjected to flash-column chromatography (SiO2, gradient MTBE—methanol) to afford (2-(methoxymethoxy)-4-(1-methyl-4-(trifluoromethyl)-1H-imidazol-2-yl)phenyl)methanamine (1.40 g, 4.44 mmol, 65.9% yield) as a light-yellow oil.

LCMS(ESI): [M+H]+ m/z: calcd 316.15; found 316.0.

### Step 7: The synthesis of 2-chloro-N4-(2-(methoxymethoxy)-4-(1-methyl-4-(trifluoromethyl)-1H-imidazol-2-yl)benzyl)-N5-methylpyrimidine-4,5-diamine

DIPEA (1.16 g, 9.01 mmol, 1.57 mL) and 2,4-dichloro-N-methyl-pyrimidin-5-amine (802 mg, 4.50 mmol) were added to a stirred solution of [2-(methoxymethoxy)-4-[1-methyl-4-(trifluoromethyl)imidazol-2-yl]phenyl]methanamine (1.42 g, 4.50 mmol) in DMF (20 mL) under argon atmosphere. The reaction mixture was stirred at 100° C. for 18 hr. The reaction mixture was cooled to room temperature and concentrated under reduced pressure. The residue was diluted with EtOAc (20 mL) and washed with water (20 mL). The organic layer was dried over anhydrous sodium sulfate and concentrated under reduced pressure to afford 2-chloro-N4-(2-(methoxymethoxy)-4-(1-methyl-4-(trifluoromethyl)-1H-imidazol-2-yl)benzyl)-N5-methylpyrimidine-4,5-diamine (1.55 g, 3.39 mmol, 75.3% yield) as a yellow solid which was used in the next steps without further purification.

LCMS(ESI): [M+H]+ m/z: calcd 457.16; found 457.2.

### Step 8: The synthesis of 2-chloro-9-(2-(methoxymethoxy)-4-(1-methyl-4-(trifluoromethyl)-1H-imidazol-2-yl)benzyl)-7-methyl-7H-purin-8(9H)-imine

A solution of potassium cyanide (273 mg, 4.19 mmol) in water (4.0 mL) was added to a stirred solution of Br2 (670 mg, 4.19 mmol) in water (1.0 mL) at 0° C. The resulting mixture was stirred at 0° C. for 15 min. A solution of 2-chloro-N4-[[2-(methoxymethoxy)-4-[1-methyl-4-(trifluoromethyl)imidazol-2-yl]phenyl]methyl]-N5-methyl-pyrimidine-4,5-diamine (500 mg, 1.09 mmol) in methanol (25 mL) was added to the mixture at 0° C. The reaction mixture was stirred at 50° C. for 36 hr. The reaction mixture was cooled to room temperature and concentrated under reduced pressure to afford 2-chloro-9-(2-(methoxymethoxy)-4-(1-methyl-4-(trifluoromethyl)-1H-imidazol-2-yl)benzyl)-7-methyl-7H-purin-8(9H)-imine (900 mg, crude) as a yellow solid which was used in the next steps without further purification.

LCMS(ESI): [M+H]+ m/z: calcd 482.15; found 482.2.

### Step 9: The synthesis of 2-(4-cyclopropyl-6-methoxy-pyrimidin-5-yl)-9-[[2-(methoxymethoxy)-4-[1-methyl-4-(trifluoromethyl)imidazol-2-yl]phenyl]methyl]-7-methyl-purin-8-imine

4-cyclopropyl-6-methoxy-5-(4,4,5,5-tetramethyl-1,3,2-dioxaborolan-2-yl)pyrimidine (86.0 mg, 311 μmol) and sodium carbonate (99.0 mg, 934 μmol) were added to a stirred solution of 2-chloro-9-[[2-(methoxymethoxy)-4-[1-methyl-4-(trifluoromethyl)imidazol-2-yl]phenyl]methyl]-7-methyl-purin-8-imine (150 mg, 311 μmol) in a degassed mixture of water (6 mL) and dioxane (18 mL) under argon atmosphere.

Bis(diphenylphosphino)ferrocene]dichloropalladium(II)-DCM (12.7 mg, 15.6 μmol) was added to the reaction mixture. The resulting mixture was stirred at 95° C. for 16 hr. under argon atmosphere. The reaction mixture was cooled to room temperature and filtered through a pad of silica gel. The filtrate was concentrated under reduced. The residue was re-diluted in MeOH (20 mL). SiliaMetS® Dimercaptotriazine (100 mg) was added to the resulting solution. The resulting mixture was stirred at room temperature for 4 hr, then solids were filtered out. The filtrate was concentrated under reduced pressure to afford 2-(4-cyclopropyl-6-methoxy-pyrimidin-5-yl)-9-[[2-(methoxymethoxy)-4-[1-methyl-4-(trifluoromethyl)imidazol-2-yl]phenyl]methyl]-7-methyl-purin-8-imine (200 mg, crude) as a brown solid which was used in the next steps without further purification.

LCMS(ESI): [M+H]+ m/z: calcd 596.26; found 596.2.

### Step 10: The synthesis of 2-[[2-(4-cyclopropyl-6-methoxy-pyrimidin-5-yl)-8-imino-7-methyl-purin-9-yl]methyl]-5-[I-methyl-4-(trifluoromethyl)imidazol-2-yl]phenol

Thionyl chloride (150 mg, 1.26 mmol, 92 μL) was added dropwise to a solution of 2-(4-cyclopropyl-6-methoxy-pyrimidin-5-yl)-9-[[2-(methoxymethoxy)-4-[1-methyl-4-(trifluoromethyl)imidazol-2-yl]phenyl]methyl]-7-methyl-purin-8-imine (150 mg, 252 μmol) in MeOH (15 mL). The reaction mixture was stirred at room temperature for 16 hr. Thionyl chloride (150 mg, 1.26 mmol, 92 μL) was added to the mixture. The resulting mixture was stirred at room temperature for 72 hr. The reaction mixture was concentrated under reduced pressure. The residue was subjected to HPLC (0-1-6 min., 10-10-65% water—ACN, +0.1% vol. of 25% aq. NH3, flow: 30 mL/min, column: YMC Triart C18 100×20 mm, 5 μm), then reputified by HPLC (0-1-6 min., 5-5-40% water -ACN, +0.1% vol. of 25% aq. NH3, flow: 30 mL/min, column: YMC Triart C18 100×20 mm, 5 μm) to afford 2-[[2-(4-cyclopropyl-6-methoxy-pyrimidin-5-yl)-8-imino-7-methyl-purin-9-yl]methyl]-5-[1-methyl-4-(trifluoromethyl)imidazol-2-yl]phenol (4.90 mg, 8.88 μmol, 6.53% yield from 2-chloro-N4-[[2-(methoxymethoxy)-4-[1-methyl-4-(trifluoromethyl)imidazol-2-yl]phenyl]methyl]-N5-methyl-pyrimidine-4,5-diamine) an off-white solid.

¹H NMR (600 MHz, DMSO-d6) δ 0.81-0.87 (m, 2H), 1.01-1.05 (m, 2H), 1.67-1.74 (m, 1H), 3.43 (s, 3H), 3.72 (s, 3H), 3.83 (s, 3H), 5.03 (s, 2H), 7.07 (d, 1H), 7.15 (s, 1H), 7.24 (br., 1H), 7.52 (br., 1H), 7.88 (s, 1H), 8.32-8.40 (m, 1H), 8.64 (s, 1H).

LCMS(ESI): [M+H]+ m/z: calcd 552.23; found 552.2.

### Example T-099

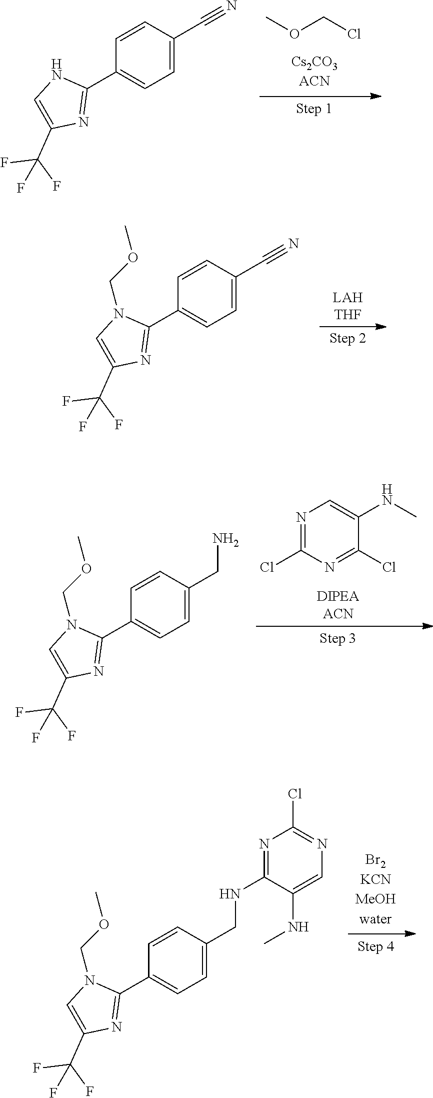

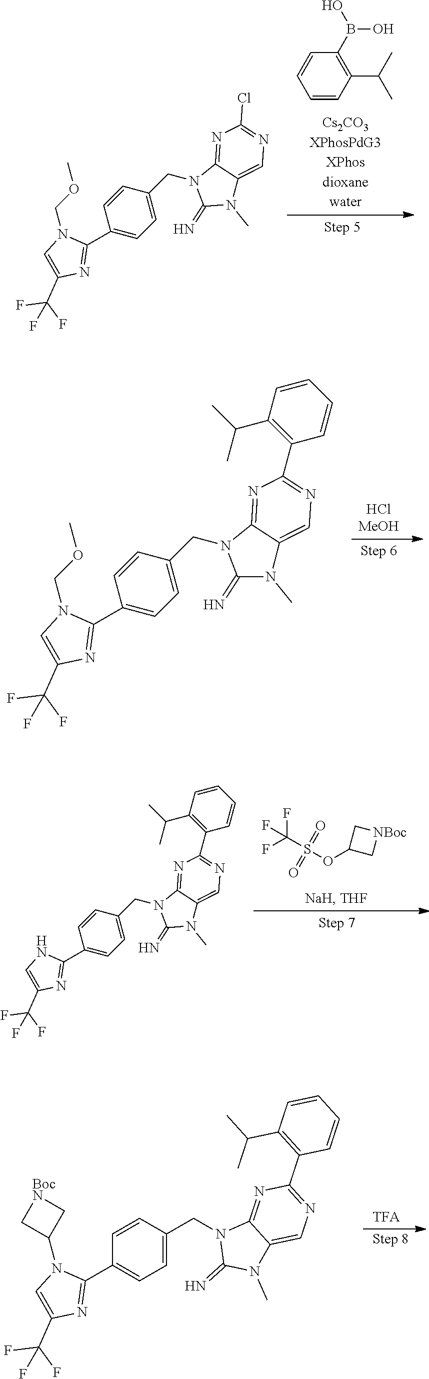

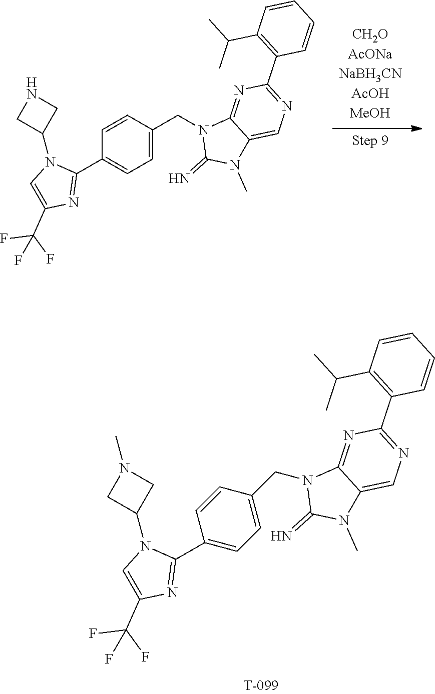 [SMILES: CC(C)c1ccccc1-c1ncc2c(n1)n(Cc1ccc(-c3nc(C(F)(F)F)cn3C3CNC3)cc1)c(=N)n2C ; CC(C)c1ccccc1-c1ncc2c(n1)n(Cc1ccc(-c3nc(C(F)(F)F)cn3C3CN(C)C3)cc1)c(=N)n2C]

### Step 1: The synthesis of 4-(1-(methoxymethyl)-4-(trifluoromethyl)-1H-imidazol-2-yl)benzonitrile

Cesium carbonate (19.2 g, 59.0 mmol) and a solution of chloromethyl methyl ether (2.61 g, 32.5 mmol) in ACN (5.0 mL) were added to a solution of 4-(4-(trifluoromethyl)-1H-imidazol-2-yl)benzonitrile (7.00 g, 29.5 mmol) in ACN (150 mL). The reaction mixture was stirred at room temperature for 12 hr. The reaction mixture was concentrated under reduced pressure. The residue was diluted with water (30 mL) and extracted with DCM (100 mL). The organic layer was washed with brine (15 mL), dried over anhydrous sodium sulfate and concentrated under reduced pressure to afford 4-(1-(methoxymethyl)-4-(trifluoromethyl)-1H-imidazol-2-yl)benzonitrile (8.00 g, 28.5 mmol, 96.4% yield) as a yellow solid which was used in the next steps without further purification.

LCMS(ESI): [M+H]+ m/z: calcd 282.10; found 282.2.

### Step 2: The synthesis of (4-(1-(methoxymethyl)-4-(trifluoromethyl)-1H-imidazol-2-yl)phenyl)methanamine

A solution of 4-[1-(methoxymethyl)-4-(trifluoromethyl)imidazol-2-yl]benzonitrile (8.00 g, 28.5 mmol) in THE (50 mL) was added dropwise to vigorously stirred solution of LAH (2.38 g, 62.6 mmol) in THE (200 mL) at 0° C. The reaction mixture was stirred at ambient temperature for 24 hr. The reaction mixture was quenched by dropwise addition of water (2.4 mL) in THE (9.0 mL), followed by an aqueous solution of NaOH (2.4 mL, 15% wt.) and water (6.0 mL). The mixture was filtered. The filtrate was concentrated under reduced pressure to afford (4-(1-(methoxymethyl)-4-(trifluoromethyl)-1H-imidazol-2-yl)phenyl)methanamine (8.00 g, 28.0 mmol, 98.6% yield) as a yellow oil which was used in the next steps without further purification.

¹H NMR (400 MHz, CDCl3) δ 3.40 (s, 3H), 3.94 (s, 2H), 5.24 (s, 2H), 7.40-7.49 (m, 3H), 7.73 (d, 2H).

LCMS(ESI): [M+H]+ m/z: calcd 286.14; found 286.2.

### Step 3: The synthesis of 2-chloro-N4-(4-(1-(methoxymethyl)-4-(trifluoromethyl)-1H-imidazol-2-yl)benzyl)-N5-methylpyrimidine-4,5-diamine

DIPEA (2.72 g, 21.0 mmol, 3.66 mL) and 2,4-dichloro-N-methyl-pyrimidin-5-amine (1.50 g, 8.41 mmol) were added to a stirred solution of (4-(1-(methoxymethyl)-4-(trifluoromethyl)-1H-imidazol-2-yl)phenyl)methanamine (2.00 g, 7.01 mmol) in ACN (20 mL) under argon atmosphere. The reaction mixture was stirred at 90° C. for 12 hr. The reaction mixture was cooled to room temperature, diluted with EtOAc (15 mL) and washed with water (2×10 mL). The organic layer was dried over anhydrous sodium sulfate and concentrated under reduced pressure. The residue was subjected to flash column chromatography (SiO2, gradient chloroform -acetonitrile) to afford 2-chloro-N4-(4-(1-(methoxymethyl)-4-(trifluoromethyl)-1H-imidazol-2-yl)benzyl)-N5-methylpyrimidine-4,5-diamine (2.78 g, 6.51 mmol, 93.0% yield) as a light-yellow gum.

LCMS(ESI): [M+H]+ m/z: calcd 427.15; found 427.0.

### Step 4: The synthesis of 2-chloro-9-(4-(1-(methoxymethyl)-4-(trifluoromethyl)-1H-imidazol-2-yl)benzyl)-7-methyl-7H-purin-8(9H)-imine

A solution of potassium cyanide (2.33 g, 35.8 mmol) in water (10.0 mL) was added to a stirred solution of Br2 (5.20 g, 32.6 mmol) in water (500 μL) at 0° C. The resulting mixture was stirred at 0° C. for 15 min. A solution of 2-chloro-N4-[[4-[1-(methoxymethyl)-4-(trifluoromethyl)imidazol-2-yl]phenyl]methyl]-N5-methyl-pyrimidine-4,5-diamine (2.78 g, 6.51 mmol) in MeOH (20 mL) was added to the mixture at 0° C. The reaction mixture was stirred at 50° C. for 12 hr. The reaction mixture was cooled to room temperature and concentrated under reduced pressure. The residue was triturated with MeOH (20 mL). The solids were filtered out. The filtrate was concentrated under reduced pressure to afford 2-chloro-9-(4-(1-(methoxymethyl)-4-(trifluoromethyl)-1H-imidazol-2-yl)benzyl)-7-methyl-7H-purin-8(9H)-imine (3.00 g, 5.63 mmol, 86.5% yield, HBr) as a light-yellow solid which was used in the next steps without further purification.

¹H NMR (400 MHz, DMSO-d6) δ 3.31 (s, 3H), 3.77 (s, 3H), 5.37 (s, 2H), 5.53 (s, 2H), 7.53 (d, 2H), 7.78 (d, 2H), 8.97 (s, 1H), 9.97 (br., 2H).

LCMS(ESI): [M+H]+ m/z: calcd 452.14; found 452.0.

### Step 5: The synthesis of 2-(2-isopropylphenyl)-9-(4-(1-(methoxymethyl)-4-(trifluoromethyl)-1H-imidazol-2-yl)benzyl)-7-methyl-7H-purin-8(9H)-imine

2-chloro-9-[[4-[1-(methoxymethyl)-4-(trifluoromethyl)imidazol-2-yl]phenyl]methyl]-7-methyl-purin-8-imine (1.72 g, 3.23 mmol, HBr), (2-isopropylphenyl)boronic acid (1.56 g, 9.52 mmol), XPhosPdG3 (242 mg, 286 μmol) and cesium carbonate (4.34 g, 13.3 mmol) were mixed in a degassed mixture of dioxane (50 mL) and water (10 mL) under argon atmosphere at room temperature. The reaction mixture was stirred at 100° C. for 12 hr. The reaction mixture was cooled to room temperature and filtered. SiliaMetS® Dimercaptotriazine (200 mg) was added to the filtrate. The resulting mixture was stirred at room temperature for 1 hr. The mixture was filtered. The filtrate was concentrated under reduced pressure. The residue was subjected to flash-column chromatography (SiO2, gradient chloroform—acetonitrile) to afford 2-(2-isopropylphenyl)-9-(4-(1-(methoxymethyl)-4-(trifluoromethyl)-1H-imidazol-2-yl)benzyl)-7-methyl-7H-purin-8(9H)-imine (900 mg, 1.68 mmol, 44.1% yield) as a yellow solid.

LCMS(ESI): [M+H]+ m/z: calcd 536.28; found 536.2.

### Step 6: The synthesis of 2-(2-isopropylphenyl)-7-methyl-9-(4-(4-(trifluoromethyl)-1H-imidazol-2-yl)benzyl)-7H-purin-8(9H)-imine

An aqueous solution of hydrogen chloride (1.82 g, 14.9 mmol, 10 M) was added to a solution of 2-(2-isopropylphenyl)-9-[[4-[1-(methoxymethyl)-4-(trifluoromethyl)imidazol-2-yl]phenyl]methyl]-7-methyl-purin-8-imine (1.60 g, 2.99 mmol) in MeOH (30 mL). The reaction mixture was stirred at 50° C. for 12 hr. The reaction mixture was cooled to room temperature and concentrated under reduced pressure. The residue was subjected to flash-column chromatography (SiO2, gradient MTBE—methanol) to afford 2-(2-isopropylphenyl)-7-methyl-9-(4-(4-(trifluoromethyl)-1H-imidazol-2-yl)benzyl)-7H-purin-8(9H)-imine (600 mg, 1.22 mmol, 40.9% yield) as a yellow solid.

LCMS(ESI): [M+H]+ m/z: calcd 492.25; found 492.2.

### Step 7: The synthesis of tert-butyl 3-(2-(4-((8-imino-2-(2-isopropylphenyl)-7-methyl-7H-purin-9(8H)-yl)methyl)phenyl)-4-(trifluoromethyl)-1H-imidazol-1-yl)azetidine-1-carboxylate

Sodium hydride (19.5 mg, 488 μmol, 60% in mineral oil) and a solution of tert-butyl 3-(trifluoromethylsulfonyloxy)azetidine-1-carboxylate (186 mg, 610 μmol) in THE (3.0 mL) were added to a solution of 2-(2-isopropylphenyl)-7-methyl-9-[[4-[4-(trifluoromethyl)-1H-imidazol-2-yl]phenyl]methyl]purin-8-imine (200 mg, 407 μmol) in THE (5.0 mL) sequentially. The reaction mixture was stirred at ambient temperature for 24 hr. The reaction mixture was diluted with water (10 mL) and extracted with MTBE (15 mL). The organic layer was washed with brine (10 mL), dried over anhydrous sodium sulfate and concentrated under reduced pressure. The residue was subjected to HPLC (0-5 min., 30-80% water—ACN, +0.1% vol. of 25% aq. NH3, flow: 30 mL/min, column: XBridge C18 100×19 mm, 5 μm) to afford tert-butyl 3-(2-(4-((8-imino-2-(2-isopropylphenyl)-7-methyl-7H-purin-9(8H)-yl)methyl)phenyl)-4-(trifluoromethyl)-1H-imidazol-1-yl)azetidine-1-carboxylate (130 mg, 201 μmol, 49.4% yield) as a light-yellow gum.

LCMS(ESI): [M+H]+ m/z: calcd 647.36; found 647.2.

### Step 8: The synthesis of 9-(4-(1-(azetidin-3-yl)-4-(trifluoromethyl)-H-imidazol-2-yl)benzyl)-2-(2-isopropylphenyl)-7-methyl-7H-purin-8(9H)-imine

A solution of tert-butyl 3-[2-[4-[[8-imino-2-(2-isopropylphenyl)-7-methyl-purin-9-yl]methyl]phenyl]-4-(trifluoromethyl)imidazol-1-yl]azetidine-1-carboxylate (130 mg, 201 μmol) in TFA (1.00 g, 8.77 mmol, 676 μL) was stirred at ambient temperature for 12 hr. The reaction mixture was concentrated under reduced pressure to afford 9-(4-(1-(azetidin-3-yl)-4-(trifluoromethyl)-1H-imidazol-2-yl)benzyl)-2-(2-isopropylphenyl)-7-methyl-7H-purin-8(9H)-imine (130 mg, crude, TFA) as a light-yellow gum which was used in the next steps without further purification.

LCMS(ESI): [M+H]+ m/z: calcd 547.30; found 547.2.

### Step 9: The synthesis of 2-(2-isopropylphenyl)-7-methyl-9-(4-(1-(1-methylazetidin-3-yl)-4-(trifluoromethyl)-1H-imidazol-2-yl)benzyl)-7H-purin-8(9H)-imine

Sodium acetate (16.1 mg, 197 μmol), formaldehyde (590 μmol, 44.4 μL, 37% wt.) and sodium cyanoborohydride (49.5 mg, 787 μmol) were added to a stirred solution of 3-(2-(4-((8-imino-2-(2-isopropylphenyl)-7-methyl-7H-purin-9(8H)-yl)methyl)phenyl)-4-(trifluoromethyl)-1H-imidazol-1-yl)azetidin-1-ium 2,2,2-trifluoroacetate (130 mg, crude, TFA) in MeOH (3.0 mL). The reaction mixture was stirred at room temperature for 24 hr. The reaction mixture was concentrated under reduced pressure. The residue was diluted with a solution of aqueous K2CO3 (5.0 mL, 10% wt.) and extracted with EtOAc (2×10 mL). The combined organic layers were dried over anhydrous sodium sulfate and concentrated under reduced pressure. The residue was subjected to HPLC (0-5 min., 35-85% water—MeOH, +0.1% vol. of 25% aq. NH3, flow: 30 mL/min, column: XBridge C18 100×19 mm, 5 μm) to afford 2-(2-isopropylphenyl)-7-methyl-9-(4-(1-(1-methylazetidin-3-yl)-4-(trifluoromethyl)-1H-imidazol-2-yl)benzyl)-7H-purin-8(9H)-imine (16.0 mg, 28.5 μmol, 14.8% yield from tert-butyl 3-[2-[4-[[8-imino-2-(2-isopropylphenyl)-7-methyl-purin-9-yl]methyl]phenyl]-4-(trifluoromethyl)imidazol-1-yl]azetidine-1-carboxylate) as a yellow gum.

¹H NMR (500 MHz, DMSO-d6) δ 1.08 (d, 6H), 2.25 (s, 3H), 3.25 (t, 2H), 3.39 (s, 3H), 3.39-3.46 (m, 1H), 3.57 (t, 2H), 4.78-4.85 (m, 1H), 5.17 (s, 2H), 7.22 (t, 1H), 7.33-7.42 (m, 2H), 7.44-7.53 (m, 5H), 8.24 (s, 1H), 8.27 (s, 1H).

LCMS(ESI): [M+H]+ m/z: calcd 561.32; found 562.2.

### Example T-112

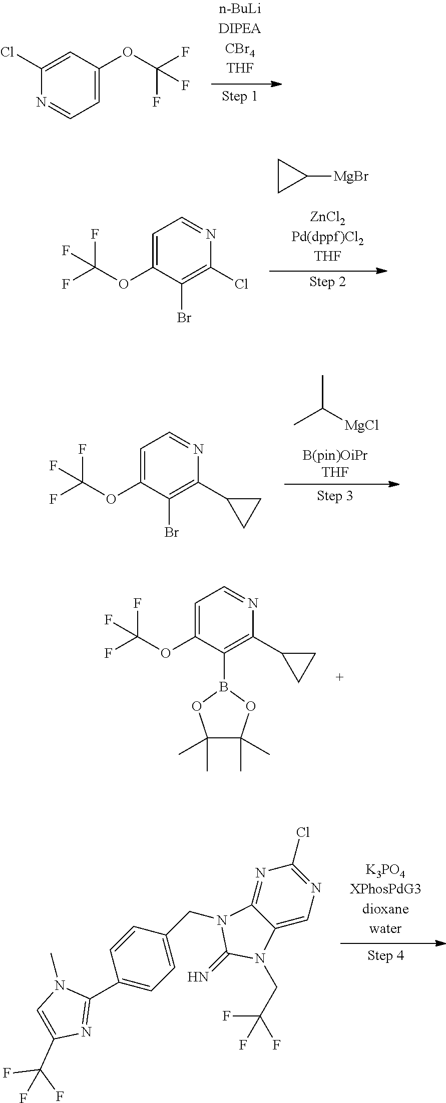

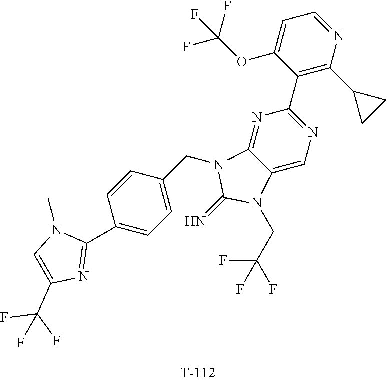 [SMILES: Cn1cc(C(F)(F)F)nc1-c1ccc(Cn2c(=N)n(CC(F)(F)F)c3cnc(-c4c(OC(F)(F)F)ccnc4C4CC4)nc32)cc1]

### Step 1: The synthesis of 3-bromo-2-chloro-4-(trifluoromethoxy)pyridine

n-Butyllithium (53.2 mmol, 21.3 mL, 2.5M in hexane) was added dropwise to a solution of diisopropylamine (5.38 g, 53.2 mmol, 7.49 mL) at −70° C. in THE (70 mL). A solution of 2-chloro-4-(trifluoromethoxy)pyridine (7.00 g, 35.4 mmol) in THE (10 mL) was added dropwise to the resulting mixture. The reaction mixture was stirred at −70° C. for 30 min. A solution of carbon tetrabromide (17.6 g, 53.2 mmol) in THE (10 mL) was added to the reaction mixture. The resulting mixture was allowed to warm to room temperature, quenched by dropwise addition of an aqueous solution of sodium bisulfate (50 mL, 5% wt.) and extracted with MTBE (100 mL). The organic layer was separated, dried over anhydrous sodium sulfate and concentrated under reduced pressure. The residue was distilled (b. p.=35° C. at 0.3 mbar) to afford 3-bromo-2-chloro-4-(trifluoromethoxy)pyridine (7.00 g, 25.3 mmol, 71.5% yield) as a light-yellow oil which was used in the next steps without further purification.

¹H NMR (600 MHz, DMSO-d6) δ 7.16 (d, 1H), 8.34 (d, 1H).

GCMS: [M]⁺ m/z: calcd 276.89, 274.90; found 277, 275.

### Step 2: The synthesis of 3-bromo-2-cyclopropyl-4-(trifluoromethoxy)pyridine

Cyclopropylmagnesium bromide (1.74 mmol, 0.5M in THF, 3.5 mL) and zinc chloride (237 mg, 1.74 mmol) were mixed in THE (1 mL) under argon atmosphere. The resulting mixture was stirred at room temperature for 30 min. 3-bromo-2-chloro-4-(trifluoromethoxy)pyridine (300 mg, 1.09 mmol) and bis(diphenylphosphino)ferrocene]dichloropalladium(II)-DCM (88.3 mg, 109 mol) were added to the mixture. The reaction mixture was stirred at 80° C. for 12 hr. The reaction mixture was cooled to room temperature, quenched by addition of an aqueous solution of NH4OH (≈700 μl, 25% wt.) and filtered through a pad of silica gel. The filtrate was concentrated under reduced pressure to afford 3-bromo-2-cyclopropyl-4-(trifluoromethoxy)pyridine (220 mg, 780 mol, 71.9% yield) as a yellow oil which was used in the next steps without further purification.

¹H NMR (400 MHz, CDCl3) δ 1.02-1.13 (m, 4H), 2.59-2.65 (m 1H), 6.97 (d, 1H), 8.32 (d, 1H).

### Step 3: The synthesis of 2-cyclopropyl-3-(4,4,5,5-tetramethyl-1,3,2-dioxaborolan-2-yl)-4-(trifluoromethoxy)pyridine

A solution of 3-bromo-2-cyclopropyl-4-(trifluoromethoxy)pyridine (100 mg, 355 μmol) in THE (850 μL) was added to isopropyl magnesium chloride (2.64 mmol, 2.2 mL, 1.2M in THF) at room temperature. The resulting mixture was stirred at room temperature for 30 min then cooled to −80° C. 2-isopropoxy-4,4,5,5-tetramethyl-1,3,2-dioxaborolane (92.4 mg, 496 μmol) was added to the mixture at −80° C. The reaction mixture was allowed to warm to room temperature and stirred at room temperature for 12 hr. The reaction mixture was quenched by addition of water (100 μL), diluted with MTBE (15 mL) and filtered through a pad of silica gel. The filtrate was washed with a solution of aqueous potassium carbonate (20 mL, 15%), dried over anhydrous sodium sulfate and concentrated under reduced pressure to afford 2-cyclopropyl-3-(4,4,5,5-tetramethyl-1,3,2-dioxaborolan-2-yl)-4-(trifluoromethoxy)pyridine (100 mg, 304 μmol, 85.7% yield) as a yellow oil which was used in the next steps without further purification.

GCMS: [M]⁺ m/z: calcd 329.14; found 329.

### Step 4: The synthesis of 2-[2-cyclopropyl-4-(trifluoromethoxy)-3-pyridyl]-9-[[4-[I-methyl-4-(trifluoromethyl)imidazol-2-yl]phenyl]methyl]-7-(2,2,2-trifluoroethyl)purin-8-imine

2-Chloro-9-[[4-[1-methyl-4-(trifluoromethyl)imidazol-2-yl]phenyl]methyl]-7-(2,2,2-trifluoroethyl)purin-8-imine (70.0 mg, 143 μmol), 2-cyclopropyl-3-(4,4,5,5-tetramethyl-1,3,2-dioxaborolan-2-yl)-4-(trifluoromethoxy)pyridine (94.1 mg, 286 μmol), potassium phosphate tribasic anhydrous (60.7 mg, 286 μmol) and XPhosPdG3 (12.1 mg, 14.3 μmol) were mixed in degassed mixture of dioxane (4.0 mL) and water (0.4 mL) under argon atmosphere. The reaction mixture was stirred at 80° C. for 72 hr. The reaction mixture was cooled to room temperature. SiliaMetS® Dimercaptotriazine (100 mg) were added to the reaction mixture. The resulting mixture was stirred at room temperature for 3 hr. The mixture was diluted with MTBE (5.0 mL) and filtered through a pad of silica gel. The filtrate was concentrated under reduced pressure. The residue was subjected to HPLC (2-10 min, 30-80% ACN+FA (0.1% vol.); flow: 30 mL/min, column: SunFire C18, 100×19 mm, 5 μm) to afford 2-[2-cyclopropyl-4-(trifluoromethoxy)-3-pyridyl]-9-[[4-[1-methyl-4-(trifluoromethyl)imidazol-2-yl]phenyl]methyl]-7-(2,2,2-trifluoroethyl)purin-8-imine (2.00 mg, 3.05 μmol, 2.13% yield) as a yellow oil.

¹H NMR (600 MHz, DMSO-d6) δ 0.77-0.83 (m, 2H), 0.95-0.99 (m, 2H), 1.72-1.80 (m, 1H), 3.74 (s, 3H), 4.80-5.06 (m, 2H), 5.09-5.27 (m, 2H), 7.10-7.19 (m, 1H), 7.25-7.29 (m, 1H), 7.47 (d, 2H), 7.62-7.68 (m, 2H), 7.92 (s, 1H), 8.36-8.43 (m, 1H), 8.56 (d, 1H).

LCMS(ESI): [M+H]⁺ m/z: calcd 657.21; found 657.2.

### Example T-139

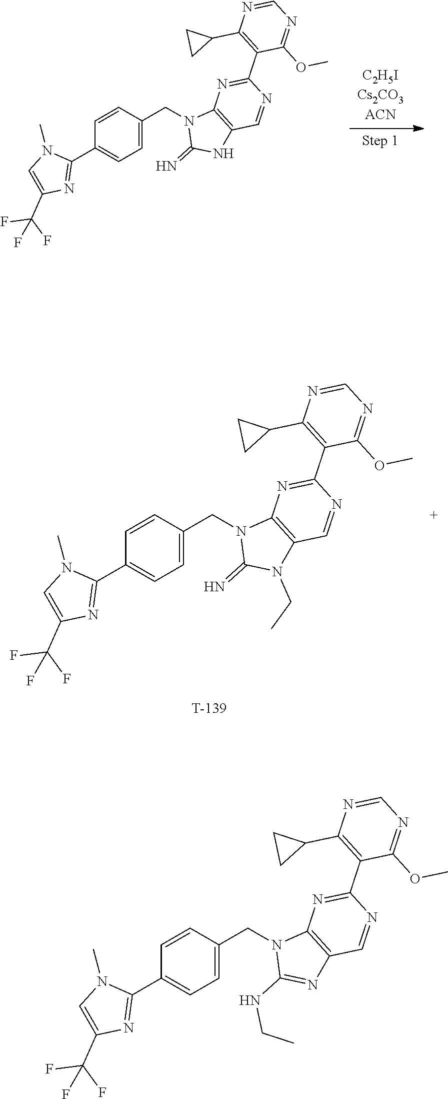 [SMILES: COc1ncnc(C2CC2)c1-c1ncc2[nH]c(=N)n(Cc3ccc(-c4nc(C(F)(F)F)cn4C)cc3)c2n1 ; CCn1c(=N)n(Cc2ccc(-c3nc(C(F)(F)F)cn3C)cc2)c2nc(-c3c(OC)ncnc3C3CC3)ncc21 ; CCNc1nc2cnc(-c3c(OC)ncnc3C3CC3)nc2n1Cc1ccc(-c2nc(C(F)(F)F)cn2C)cc1]

### Step 1: The synthesis of 2-(4-cyclopropyl-6-methoxy-pyrimidin-5-yl)-7-ethyl-9-[[4-[-methyl-4-(trifluoromethyl)imidazol-2-yl]phenyl]methyl]purin-8-imine

Iodoethane (65.8 mg, 422 μmol, 34 μL) was added to a stirred mixture of 2-(4-cyclopropyl-6-methoxy-pyrimidin-5-yl)-9-[[4-[1-methyl-4-(trifluoromethyl)imidazol-2-yl]phenyl]methyl]-7H-purin-8-imine (200 mg, 384 μmol) and cesium carbonate (187 mg, 575 mol) in ACN (5.0 mL). The reaction mixture was stirred at 40° C. for 12 hr. The reaction mixture was cooled to room temperature, diluted with EtOAc (10 mL) and washed with water (5.0 mL) and brine (5.0 mL). The organic layer was concentrated under reduced pressure. The residue was subjected to HPLC (0.5-6.5 min., 26% water—ACN, +0.1% vol. of 25% aq. NH3, flow: 30 mL/min, column: XBridge 100×19 mm, 5 μm), then repurified by HPLC (0.5-6.5 min., 40-90% water—MeOH, +0.1% vol. of 25% aq. NH3, flow: 30 mL/min, column: XBridge 100×19 mm, 5 μm), then repurified by HPLC (0.5-6.5 min, 15-30% water+FA (0.1% vol.)—ACN+FA (0.1% vol.); flow: 30 mL/min, column: SunFire C18, 100×19 mm, 5 μm) to afford 2-(4-cyclopropyl-6-methoxy-pyrimidin-5-yl)-7-ethyl-9-[[4-[1-methyl-4-(trifluoromethyl)imidazol-2-yl]phenyl]methyl]purin-8-imine (7.00 mg, 12.7 μmol, 3.32% yield) as an off-white solid and 2-(4-cyclopropyl-6-methoxy-pyrimidin-5-yl)-N-ethyl-9-[[4-[1-methyl-4-(trifluoromethyl)imidazol-2-yl]phenyl]methyl]purin-8-amine (8 mg, 14.56 μmol, 3.80% yield) as an off-white solid.

### 2-(4-cyclopropyl-6-methoxy-pyrimidin-5-yl)-7-ethyl-9-[[4-[1-methyl-4-(trifluoromethyl)imidazol-2-yl]phenyl]methyl]purin-8-imine

¹H NMR (600 MHz, DMSO-d6) δ 0.78-0.84 (m, 2H), 0.96-1.01 (m, 2H), 1.22 (t, 3H), 1.66-1.72 (m, 1H), 3.73 (s, 3H), 3.81 (s, 3H), 3.92 (q, 2H), 5.13 (br., 2H), 7.47 (d, 2H), 7.64 (d, 2H), 7.90 (s, 1H), 8.61 (s, 1H).

LCMS(ESI): [M+H]⁺ m/z: calcd 550.26; found 550.2.

### 2-(4-cyclopropyl-6-methoxy-pyrimidin-5-yl)-N-ethyl-9-[[4-[1-methyl-4-(trifluoromethyl)imidazol-2-yl]phenyl]methyl]purin-8-amine

¹H NMR (600 MHz, DMSO-d6) δ 0.75-0.80 (m, 2H), 0.95-0.99 (m, 2H), 1.20 (t, 3H), 1.59-1.65 (m, 1H), 3.43-3.50 (m, 2H), 3.72 (s, 3H), 3.80 (s, 3H), 5.36 (s, 2H), 7.37 (d, 2H), 7.61-7.68 (m, 3H), 7.90 (s, 1H), 8.58 (s, 1H), 8.61 (s, 1H).

LCMS(ESI): [M+H]⁺ m/z: calcd 550.26; found 550.2.

### Example T-103

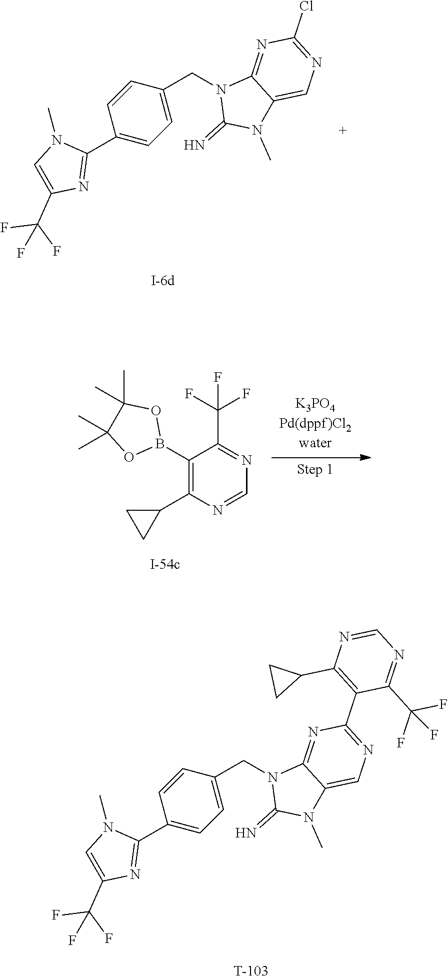 [SMILES: Cn1cc(C(F)(F)F)nc1-c1ccc(Cn2c(=N)n(C)c3cnc(Cl)nc32)cc1 ; CC1(C)OB(c2c(C3CC3)ncnc2C(F)(F)F)OC1(C)C ; Cn1cc(C(F)(F)F)nc1-c1ccc(Cn2c(=N)n(C)c3cnc(-c4c(C5CC5)ncnc4C(F)(F)F)nc32)cc1]

### Step 1: The synthesis of 2-(4-cyclopropyl-6-(trifluoromethyl)pyrimidin-5-yl)-7-methyl-9-(4-(1-methyl-4-(trifluoromethyl)-1H-imidazol-2-yl)benzyl)-7H-purin-8(9H)-imine

The synthesis of the starting 4-cyclopropyl-5-(4,4,5,5-tetramethyl-1,3,2-dioxaborolan-2-yl)-6-(trifluoromethyl)pyrimidine (I-54c) is described in Intermediate 54. [1203] (4-cyclopropyl-5-(4,4,5,5-tetramethyl-1,3,2-dioxaborolan-2-yl)-6-(trifluoromethyl)pyrimidine (200 mg, 637 μmol), 2-chloro-7-methyl-9-(4-(1-methyl-4-(trifluoromethyl)-1H-imidazol-2-yl)benzyl)-7H-purin-8(9H)-imine (60.0 mg, 142 μmol), bis(diphenylphosphino)ferrocene]dichloropalladium(II)-DCM (50.0 mg, 61.2 μmol) and potassium phosphate tribasic anhydrous (406 mg, 1.91 mmol) were mixed in a degassed mixture of dioxane (5 mL) and water (1.0 mL). The reaction mixture was stirred at 98° C. for 20 hr. under argon atmosphere. The reaction mixture was cooled to room temperature and filtered. SiliaMetS® Dimercaptotriazine (100 mg) was added to the filtrate. The resulting mixture was stirred for 30 min and filtered. The filtrate was concentrated under reduced pressure. The residue was subjected to HPLC (0-5 min., 20-70% water—ACN, +0.1% vol. of 25% aq. NH3, flow: 30 mL/min, column: YMC Triart C18 100×20 mm, 5 μm) to afford 2-(4-cyclopropyl-6-(trifluoromethyl)pyrimidin-5-yl)-7-methyl-9-(4-(1-methyl-4-(trifluoromethyl)-1H-imidazol-2-yl)benzyl)-7H-purin-8(9H)-imine (4.2 mg, 7.32 μmol, 1.15% yield) as a light-yellow solid.

¹H NMR (600 MHz, DMSO-d6) δ 0.98-1.03 (m, 2H), 1.11-1.16 (m, 2H), 1.77-1.82 (m, 1H), 3.37-3.44 (m, 3H), 3.74 (s, 3H), 5.08-5.25 (m, 2H), 6.66-6.79 (m, 1H), 7.42-7.48 (m, 2H), 7.61-7.67 (m, 2H), 7.92 (s, 1H), 8.25-8.34 (m, 1H), 9.23 (s, 1H).

LCMS(ESI): [M+H]+ m/z: calcd 574.22; found 574.2.

### Example T-145

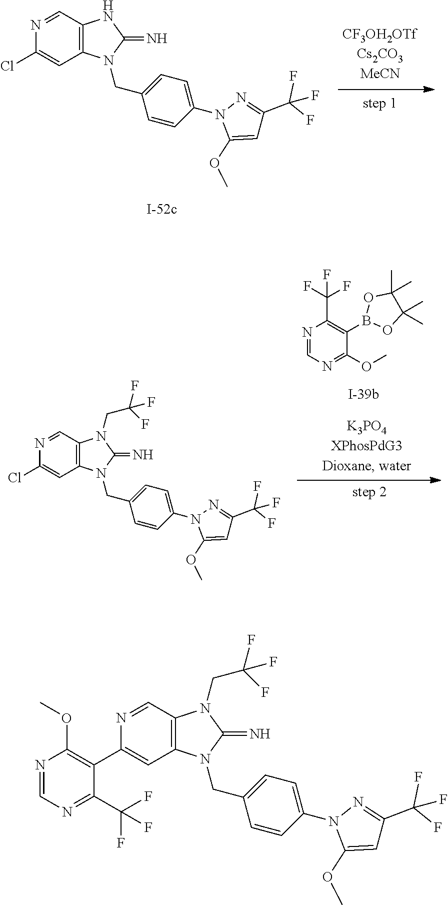 [SMILES: COc1cc(C(F)(F)F)nn1-c1ccc(Cn2c(=N)[nH]c3cnc(Cl)cc32)cc1 ; COc1ncnc(C(F)(F)F)c1B1OC(C)(C)C(C)(C)O1 ; COc1cc(C(F)(F)F)nn1-c1ccc(Cn2c(=N)n(CC(F)(F)F)c3cnc(Cl)cc32)cc1 ; COc1ncnc(C(F)(F)F)c1-c1cc2c(cn1)n(CC(F)(F)F)c(=N)n2Cc1ccc(-n2nc(C(F)(F)F)cc2OC)cc1]

The synthesis of4-methoxy-5-(4,4,5,5-tetramethyl-1,3,2-dioxaborolan-2-yl)-6-(trifluoromethyl)pyrimidine (I-39b) is described by Intermediate 39.

### Step 1: Synthesis of 2-chloro-9-[[4-[5-methoxy-3-(trifluoromethyl)pyrazol-1-yl]phenyl]methyl]-7-(2,2,2-trifluoroethyl)purin-8-imine

The synthesis of the starting 2-chloro-9-[[4-[5-methoxy-3-(trifluoromethyl)pyrazol-1-yl]phenyl]methyl]-7H-purin-8-imine (I-52c) is described in Intermediate 52.

To a mixture of 2-chloro-9-[[4-[5-methoxy-3-(trifluoromethyl)pyrazol-1-yl]phenyl]methyl]-7H-purin-8-imine (1.00 g, 2.36 mmol) and cesium carbonate (1.15 g, 3.54 mmol) in ACN (30 mL) 2,2,2-trifluoroethyl trifluoromethanesulfonate (603 mg, 2.60 mmol) was added one portion. The resulting mixture was stirred at 50° C. for 16 hr. The mixture was cooled to room temperature and concentrated under reduced pressure. The residue was partitioned between EtOAc (60 mL) and water (30 mL). The organic layer was separated, washed with brine (20 mL), dried over anhydrous sodium sulfate and concentrated under reduced pressure. The residue was subjected to HPLC (gradient elution: 2-10 min, 10-50% water+FA (0.1% vol.) -ACN+FA (0.1% vol.); flow: 30 mL/min, column: Waters SunFire C18, 100×19 mm, 5 μm) to afford 2-chloro-9-[[4-[5-methoxy-3-(trifluoromethyl)pyrazol-1-yl]phenyl]methyl]-7-(2,2,2-trifluoroethyl)purin-8-imine (80.0 mg, 158 μmol, 6.70% yield) as a yellow solid.

LCMS(ESI): [M+H]+ m/z: calcd 506.11; found 506.0.

### Step 2: Synthesis of 9-[[4-[5-methoxy-3-(trifluoromethyl)pyrazol-1-yl]phenyl]methyl]-2-[4-methoxy-6-(trifluoromethyl)pyrimidin-5-yl]-7-(2,2,2-trifluoroethyl)purin-8-imine

2-Chloro-9-[[4-[5-methoxy-3-(trifluoromethyl)pyrazol-1-yl]phenyl]methyl]-7-(2,2,2-trifluoroethyl)purin-8-imine (80.0 mg, 158 μmol), 4-methoxy-5-(4,4,5,5-tetramethyl-1,3,2-dioxaborolan-2-yl)-6-(trifluoromethyl)pyrimidine (96.2 mg, 316 μmol), potassium phosphate tribasic anhydrous (101 mg, 475 μmol) and XPhos Pd G3 (10.0 mg, 15.8 μmol) were mixed in degassed mixture of dioxane (4 mL) and water (0.5 mL). The reaction mixture was stirred at 90° C. for 12 hr. The reaction mixture was cooled to room temperature. The reaction mixture was diluted with EtOAc (20 mL) and washed with water (10 mL) and brine (10 mL). To the obtained organic solution SiliaMetS® Dimercaptotriazine (20 mg) was added, and the mixture was stirred for 30 min. The mixture was filtered. The filtrate was concentrated under reduced pressure. The residue was subjected to HPLC (gradient elution: 2-10 min, 40-55% water—methanol, +0.1% vol. of 25% aq. NH3, flow: 30 mL/min, column: SunFire C18 100×19 mm, 5 μm) to afford 9-[[4-[5-methoxy-3-(trifluoromethyl)pyrazol-1-yl]phenyl]methyl]-2-[4-methoxy-6-(trifluoromethyl)pyrimidin-5-yl]-7-(2,2,2-trifluoroethyl)purin-8-imine (12.0 mg, 18.5 μmol, 11.7% yield) as a light-yellow solid.

¹H NMR (600 MHz, DMSO-d6) δ 3.94 (s, 3H), 3.95 (s, 3H), 4.76-5.02 (m, 2H), 5.04-5.21 (m, 2H), 6.44 (s, 1H) 7.14-7.21 (m, 1H), 7.45 (d, 2H), 7.53-7.59 (m, 2H), 8.30-8.36 (m, 1H), 9.06 (s, 1H).

LCMS(ESI): [M+H]+ m/z: calcd 648.17; found 648.0.

### Example T-136

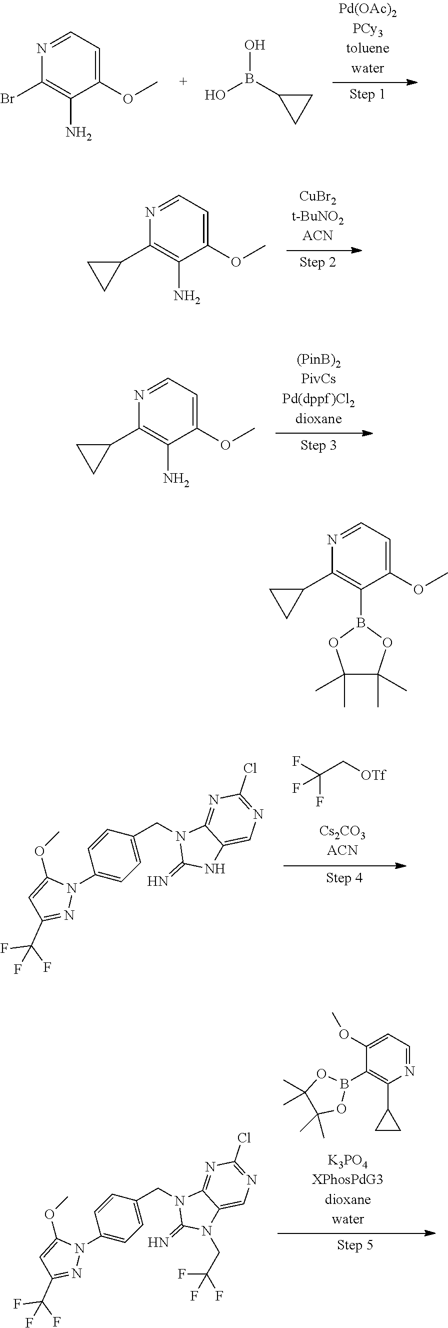

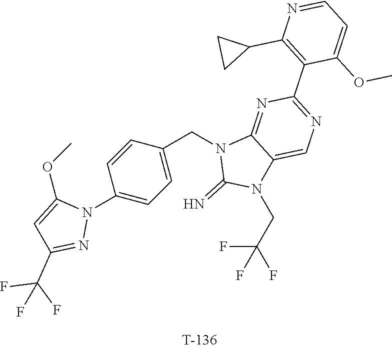 [SMILES: COc1ccnc(C2CC2)c1-c1ncc2c(n1)n(Cc1ccc(-n3nc(C(F)(F)F)cc3OC)cc1)c(=N)n2CC(F)(F)F]

### Step 1: The synthesis of 2-cyclopropyl-4-methoxy-pyridin-3-amine

2-bromo-4-methoxy-pyridin-3-amine (4.00 g, 19.7 mmol), cyclopropylboronic acid (5.42 g, 63.0 mmol), tricyclohexylphosphine (553 mg, 1.97 mmol) and potassium phosphate tribasic (12.6 g, 59.1 mmol) were mixed in a degassed mixture of toluene (50 mL) and water (10 mL). Palladium (II) acetate (332 mg, 1.48 mmol) was added to the mixture. The reaction mixture was stirred at 95° C. for 15 hr. The reaction mixture was cooled to room temperature and concentrated under reduced pressure. The residue was diluted with water (30 mL) and extracted with EtOAc (4×25 mL). The combined organic layers were dried over anhydrous sodium sulfate and concentrated under reduced pressure. The residue was subjected to flash-column chromatography (SiO2, gradient elution: MTBE—ACN) to afford 2-cyclopropyl-4-methoxy-pyridin-3-amine (2.23 g, 13.6 mmol, 68.9% yield) as a yellow solid.

¹H NMR (400 MHz, DMSO-d6) δ 0.77-0.85 (m, 4H), 2.04-2.12 (m, 1H), 3.80 (s, 3H), 4.62 (s, 2H), 6.66 (d, 1H), 7.62 (d, 1H).

LCMS(ESI): [M+H]+ m/z: calcd 165.12; found 165.4.

### Step 2: The synthesis of 3-bromo-2-cyclopropyl-4-methoxy-pyridine

2-cyclopropyl-4-methoxy-pyridin-3-amine (1.20 g, 7.31 mmol), copper (II) bromide (3.26 g, 14.6 mmol) and tert-butyl nitrite (1.21 g, 11.7 mmol, 1.39 mL) were mixed in ACN (50 mL). The reaction mixture was stirred at 85° C. for 12 hr. The reaction mixture was cooled to room temperature and concentrated under reduced pressure. The residue was diluted with brine (50 mL) and extracted with EtOAc (100 mL). The organic layer was washed with brine (50 mL), dried over anhydrous sodium sulfate and concentrated under reduced pressure. The residue was subjected to column chromatography (SiO2, Hexane—EtOAc 9/1) to afford 3-bromo-2-cyclopropyl-4-methoxy-pyridine (850 mg, 3.73 mmol, 51.0% yield) as a yellow solid.

¹H NMR (400 MHz, CDCl3) δ 0.95-1.08 (m, 4H), 2.52-2.60 (m, 1H), 3.91 (s, 3H), 6.57 (d, 1H), 8.20 (d, 1H).

LCMS(ESI): [M+H]+ m/z: calcd 228.00, 230.00; found 228.2, 230.2.

### Step 3: The synthesis of 2-cyclopropyl-4-methoxy-3-(4,4,5,5-tetramethyl-1,3,2-dioxaborolan-2-yl)pyridine

3-bromo-2-cyclopropyl-4-methoxy-pyridine (200 mg, 877 μmol), cesium pivalate (410 mg, 1.75 mmol) and bis(pinacolato)diboron (334 mg, 1.32 mmol) were mixed in degassed dioxane (5.0 mL). The resulting mixture was degassed thrice. Bis(diphenylphosphino)ferrocene]dichloropalladium(II)-DCM (71.6 mg, 87.7 μmol) was added to the mixture. The reaction mixture was stirred at 85° C. for 12 hr. The reaction mixture was cooled to room temperature and subjected to HPLC (gradient elution: 2-10 min, 30-55% ACN; flow: 30 mL/min, column: SunFire C18, 100×19 mm, 5 μm) to afford 2-cyclopropyl-4-methoxy-3-(4,4,5,5-tetramethyl-1,3,2-dioxaborolan-2-yl)pyridine (40.0 mg, 145 μmol, 16.6% yield) as a brown oil which was used in the next steps without further purification.

LCMS(ESI): [M+H]+ m/z: calcd 276.21; found 276.2.

### Step 4: The synthesis of 2-chloro-9-[[4-[5-methoxy-3-(trifluoromethyl)pyrazol-1-yl]phenyl]methyl]-7-(2,2,2-trifluoroethyl)purin-8-imine

Cesium carbonate (354 mg, 1.09 mmol) was added to a solution of 2-chloro-9-[[4-[5-methoxy-3-(trifluoromethyl)pyrazol-1-yl]phenyl]methyl]-7H-purin-8-imine (200 mg, 472 μmol) in ACN (12 mL). The resulting mixture was stirred at room temperature for 15 min. 2,2,2-trifluoroethyl trifluoromethanesulfonate (142 mg, 614 μmol, 88.4 μL) was added to the mixture. The reaction mixture was stirred at 60° C. for 16 hr. The reaction mixture was cooled to room temperature and concentrated under reduced pressure. The residue was subjected to HPLC (gradient elution: 2-10 min, 46-65% ACN+FA (0.1% vol.); flow: 30 mL/min, column: SunFire C18, 100×19 mm, 5 μm) to afford 2-chloro-9-[[4-[5-methoxy-3-(trifluoromethyl)pyrazol-1-yl]phenyl]methyl]-7-(2,2,2-trifluoroethyl)purin-8-imine (25.0 mg, 49.4 μmol, 10.5% yield) as a brown solid.

LCMS(ESI): [M+H]+ m/z: calcd 506.11; found 506.0.

### Step 5: The synthesis of 2-(2-cyclopropyl-4-methoxy-3-pyridyl)-9-[[4-[5-methoxy-3-(trifluoromethyl)pyrazol-1-yl]phenyl]methyl]-7-(2,2,2-trifluoroethyl)purin-8-imine

2-chloro-9-[[4-[5-methoxy-3-(trifluoromethyl)pyrazol-1-yl]phenyl]methyl]-7-(2,2,2-trifluoroethyl)purin-8-imine (25.0 mg, 49.4 μmol), 2-cyclopropyl-4-methoxy-3-(4,4,5,5-tetramethyl-1,3,2-dioxaborolan-2-yl)pyridine (27.2 mg, 98.9 μmol), potassium phosphate tribasic anhydrous (21.0 mg, 98.9 μmol) and XPhos Pd G3 (4.18 mg, 4.94 μmol) were mixed in degassed mixture of dioxane (2.0 mL) and water (0.2 mL) under an argon atmosphere. The reaction mixture was stirred at 80° C. for 72 hr. The reaction mixture was cooled to room temperature. SiliaMetS® Dimercaptotriazine (100 mg) was added to the reaction mixture. The resulting mixture was stirred at room temperature for 3 hr. The mixture was diluted with MTBE (5.0 mL) and filtered through a short pad of silica gel. The filtrate was concentrated under reduced pressure. The residue was subjected to HPLC (2-10 min., 35-50% ACN, +0.1% vol. of 25% aq. NH3, flow: 30 mL/min, column: SunFire 100×19 mm, 5 μm) to afford 2-(2-cyclopropyl-4-methoxy-3-pyridyl)-9-[[4-[5-methoxy-3-(trifluoromethyl)pyrazol-1-yl]phenyl]methyl]-7-(2,2,2-trifluoroethyl)purin-8-imine (14.0 mg, 22.6 μmol, 45.8% yield) as a yellow solid.

¹H NMR (600 MHz, DMSO-d6) δ 0.64-0.70 (m, 2H), 0.86-0.91 (m, 2H), 1.44-1.51 (m, 1H), 3.71 (s, 3H), 3.97 (s, 3H), 4.76-5.03 (m, 2H), 5.06-5.24 (m, 2H), 6.45 (s, 1H), 6.91 (d, 2H), 7.00-7.09 (m, 1H), 7.50 (d, 2H), 7.54-7.61 (m, 2H), 8.29-8.36 (m, 2H).

LCMS(ESI): [M+H]+ m/z: calcd 619.23; found 619.2.

### Example T-104

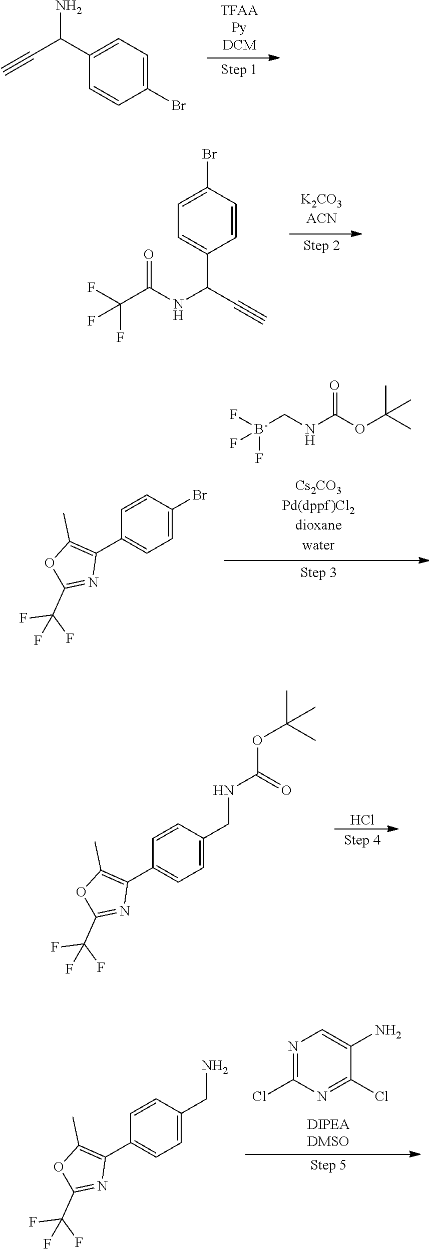

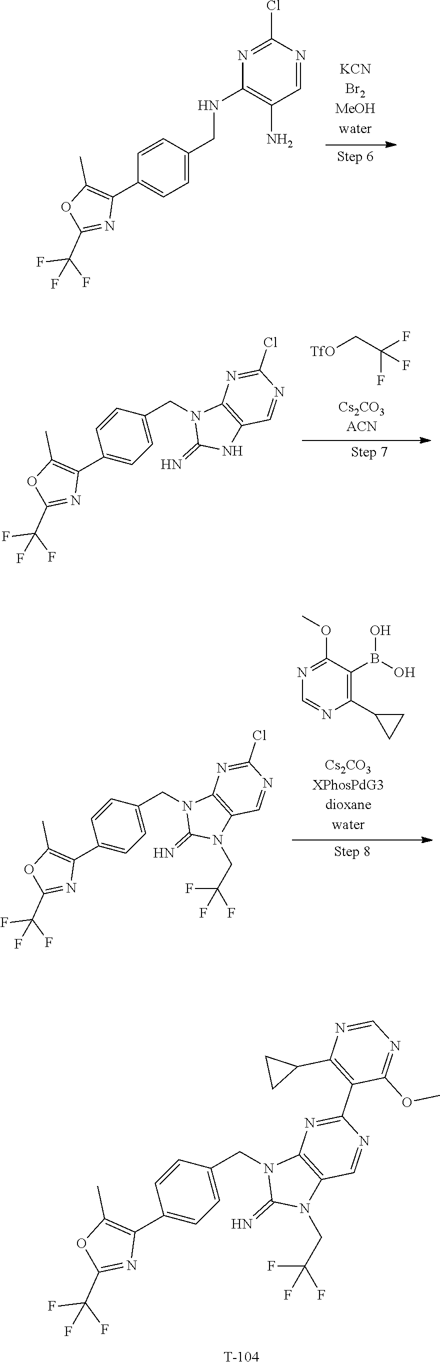 [SMILES: Cc1oc(C(F)(F)F)nc1-c1ccc(CNc2nc(Cl)ncc2N)cc1 ; Cc1oc(C(F)(F)F)nc1-c1ccc(Cn2c(=N)[nH]c3cnc(Cl)nc32)cc1 ; O=S(=O)(OCC(F)(F)F)C(F)(F)F ; Cc1oc(C(F)(F)F)nc1-c1ccc(Cn2c(=N)n(CC(F)(F)F)c3cnc(Cl)nc32)cc1 ; COc1ncnc(C2CC2)c1B(O)O ; COc1ncnc(C2CC2)c1-c1ncc2c(n1)n(Cc1ccc(-c3nc(C(F)(F)F)oc3C)cc1)c(=N)n2CC(F)(F)F]

### Step 1: The synthesis of N-[1-(4-bromophenyl)prop-2-ynyl]-2,2,2-trifluoro-acetamide

Pyridine (225 mg, 2.85 mmol, 230 μL) and trifluoroacetic anhydride (299 mg, 1.42 mmol, 201 μL) were added to a solution of 1-(4-bromophenyl)prop-2-yn-1-amine (319 mg, 1.29 mmol, HCl) in DCM (10 mL) at 0° C. The reaction mixture was stirred at room temperature for 16 hr. The reaction mixture was diluted with DCM (10 mL) and washed with brine (10 mL). The organic layer was dried over anhydrous sodium sulfate and concentrated under reduced pressure to afford N-[1-(4-bromophenyl)prop-2-ynyl]-2,2,2-trifluoro-acetamide (293 mg, 957 μmol, 74.0% yield) as a white solid which was used in the next steps without further purification.

¹H NMR (400 MHz, DMSO-d6) δ 2.61 (s, 1H), 5.92 (d, 1H), 6.68 (br, 1H), 7.39 (d, 2H), 7.53 (d, 2H).

LCMS(ESI): [M+H]+ m/z: calcd 305.99; found 307.0.

### Step 2: The synthesis of 4-(4-bromophenyl)-5-methyl-2-(trifluoromethyl)oxazole

Potassium carbonate (316 mg, 2.29 mmol) was added to a solution of N-[1-(4-bromophenyl)prop-2-ynyl]-2,2,2-trifluoro-acetamide (70.0 mg, 229 μmol) in ACN (3.0 mL). The reaction mixture was stirred at room temperature for 96 hr. The reaction mixture was concentrated under reduced pressure. The residue was diluted with DCM (20 mL) and washed with brine (10 mL). The organic layer was dried over anhydrous sodium sulfate and concentrated under reduced pressure to afford 4-(4-bromophenyl)-5-methyl-2-(trifluoromethyl)oxazole (51.0 mg, 167 μmol, 72.9% yield) as a brown oil which was used in the next steps without further purification.

¹H NMR (500 MHz, CDCl3) δ 2.61 (s, 3H), 7.54 (d, 2H), 7.59 (d, 2H).

### Step 3: The synthesis of tert-butyl N-[[4-[5-methyl-2-(trifluoromethyl)oxazol-4-yl]phenyl]methyl]carbamate

Potassium (tert-butoxycarbonylamino)methyl-trifluoro-boranuide (1.92 g, 8.09 mmol), cesium carbonate (5.27 g, 16.2 mmol) and bis(diphenylphosphino)ferrocene]dichloropalladium(II)-DCM (220 mg, 270 μmol) were added to a solution of 4-(4-bromophenyl)-5-methyl-2-(trifluoromethyl)oxazole (1.65 g, 5.39 mmol) in a degassed mixture of dioxane (40 mL) and water (10 mL) under argon atmosphere. The reaction mixture was stirred at 95° C. for 16 hr. The reaction mixture was cooled to room temperature and concentrated under reduced pressure. The residue was diluted with EtOAc (20 mL) and washed with brine (10 mL). The organic layer was dried over anhydrous sodium sulfate and concentrated under reduced pressure to afford tert-butyl N-[[4-[5-methyl-2-(trifluoromethyl)oxazol-4-yl]phenyl]methyl]carbamate (1.86 g, 5.22 mmol, 96.8% yield) as a brown oil which was used in the next steps without further purification.

LCMS(ESI): [M-tBu+H]+ m/z: calcd 301.08; found 301.2.

### Step 4: The synthesis of [4-[5-methyl-2-(trifluoromethyl)oxazol-4-yl]phenyl]methanamine

Hydrochloric acid (10 mL, 4 M in dioxane) was added to a solution of tert-butyl N-[[4-[5-methyl-2-(trifluoromethyl)oxazol-4-yl]phenyl]methyl]carbamate (1.86 g, 5.22 mmol) in diethyl ether (10 mL). The resulting solution was stirred at room temperature for 3 hr. The reaction mixture was diluted with diethyl ether (20 mL). The solid precipitate formed was filtered off, washed with diethyl ether (10 mL) and air-dried to afford [4-[5-methyl-2-(trifluoromethyl)oxazol-4-yl]phenyl]methanamine (1.17 g, 4.00 mmol, 76.6% yield, HCl) as a light-yellow solid which was used in the next steps without further purification.

¹H NMR (500 MHz, DMSO-d6) δ 2.64 (s, 3H), 4.06 (s, 2H), 7.60 (d, 2H), 7.74 (d, 2H), 8.47 (br, 2H).

### Step 5: The synthesis of 2-chloro-N4-[[4-[5-methyl-2-(trifluoromethyl)oxazol-4-yl]phenyl]methyl]pyrimidine-4,5-diamine

[4-[5-methyl-2-(trifluoromethyl)oxazol-4-yl]phenyl]methanamine (1.17 g, 4.00 mmol, HCl), 2,4-dichloropyrimidin-5-amine (983 mg, 6.00 mmol) and DIPEA (1.29 g, 9.99 mmol, 1.74 mL) were mixed in DMSO (4.0 mL). The reaction mixture was stirred at 90° C. for 14 hr. The reaction mixture was cooled to room temperature, diluted with EtOAc (40 mL) and washed with brine (60 mL). The organic layer was dried over anhydrous sodium sulfate and concentrated under reduced pressure to afford 2-chloro-N4-[[4-[5-methyl-2-(trifluoromethyl)oxazol-4-yl]phenyl]methyl]pyrimidine-4,5-diamine (1.53 g, 3.99 mmol, 99.8% yield) as a brown oil which was used in the next steps without further purification.

LCMS(ESI): [M+H]+ m/z: calcd 384.10; found 384.0.

### Step 6: The synthesis of 2-chloro-9-[[4-[5-methyl-2-(trifluoromethyl)oxazol-4-yl]phenyl]methyl]-7H-purin-8-imine

A solution of potassium cyanide (3.05 g, 46.9 mmol) in water (6.0 mL) was added to a stirred solution of molecular bromine (7.50 g, 46.9 mmol) in MeOH (50 mL) at room temperature. The resulting mixture was stirred at room temperature for 15 min. A solution of 2-chloro-N4-[[4-[5-methyl-2-(trifluoromethyl)oxazol-4-yl]phenyl]methyl]pyrimidine-4,5-diamine (1.80 g, 4.69 mmol) in MeOH (4.0 mL) was added to the mixture. The reaction mixture was stirred at room temperature for 16 hr. The reaction mixture was concentrated under reduced pressure. The residue was diluted with EtOAc (80 mL) and washed with a solution of aqueous potassium carbonate (50 mL, 10%). The organic layer was dried over anhydrous sodium sulfate and filtered. The filtrate was concentrated under reduced pressure. The residue was subjected to flash column chromatography (SiO2, gradient elution: chloroform—acetonitrile) to afford 2-chloro-9-[[4-[5-methyl-2-(trifluoromethyl)oxazol-4-yl]phenyl]methyl]-7H-purin-8-imine (530 mg, 1.30 mmol, 27.6% yield) as a yellow solid.

¹H NMR (400 MHz, DMSO-d6) δ 2.61 (s, 3H), 5.32 (s, 2H), 7.32 (d, 2H), 7.50 (s, 2H), 7.69 (d, 2H), 8.30 (s, 1H).

LCMS(ESI): [M+H]+ m/z: calcd 409.08; found 409.0.

### Step 7: The synthesis of 2-chloro-9-[[4-[5-methyl-2-(trifluoromethyl)oxazol-4-yl]phenyl]methyl]-7-(2,2,2-trifluoroethyl)purin-8-imine

2,2,2-trifluoroethyl trifluoromethanesulfonate (213 mg, 917 μmol, 132 μL) was added dropwise to a stirred mixture of 2-chloro-9-[[4-[5-methyl-2-(trifluoromethyl)oxazol-4-yl]phenyl]methyl]-7H-purin-8-imine (250 mg, 612 μmol) and cesium carbonate (399 mg, 1.22 mmol) in ACN (5.0 mL). The reaction mixture was stirred at 60° C. for 16 hr. The reaction mixture was concentrated under reduced pressure, diluted with water (30 mL) and extracted with EtOAc (40 mL). The organic layer was washed with brine (30 mL), dried over anhydrous sodium sulfate and concentrated under reduced pressure. The residue was subjected to HPLC (gradient elution: 30-90% water+FA (0.1% vol.)—ACN+FA (0.1% vol.); flow: 30 mL/min, column: Chromatorex 18 SMB100-5T, 100×19 mm, 5 μm) to afford 2-chloro-9-[[4-[5-methyl-2-(trifluoromethyl)oxazol-4-yl]phenyl]methyl]-7-(2,2,2-trifluoroethyl)purin-8-imine (25.0 mg, 50.9 μmol, 8.33% yield) as an off-white solid.

LCMS(ESI): [M+H]+ m/z: calcd 491.10; found 491.0.

### Step 8: The synthesis of 2-(4-cyclopropyl-6-methoxy-pyrimidin-5-yl)-9-[[4-[5-methyl-2-(trifluoromethyl)oxazol-4-yl]phenyl]methyl]-7-(2,2,2-trifluoroethyl)purin-8-imine

(4-Cyclopropyl-6-methoxy-pyrimidin-5-yl)boronic acid (19.8 mg, 102 μmol), cesium carbonate (49.8 mg, 153 μmol) and XPhosPdG3 (2.16 mg, 2.55 μmol) were added to a solution of 2-chloro-9-[[4-[5-methyl-2-(trifluoromethyl)oxazol-4-yl]phenyl]methyl]-7-(2,2,2-trifluoroethyl)purin-8-imine (25.0 mg, 50.9 μmol) in a degassed mixture of dioxane (2.0 mL) and water (500 μL) under an argon atmosphere. The reaction mixture was stirred at 95° C. for 16 hr. The reaction mixture was cooled to room temperature and concentrated under reduced pressure. The residue was diluted with EtOAc (20 mL) and washed with brine (10 mL). The organic layer was dried over anhydrous sodium sulfate and filtered. The filtrate was concentrated under reduced pressure. The residue was subjected to HPLC (gradient elution: 30-80% water—ACN, +0.1% vol. of 25% aq. NH3, flow: 30 mL/min, column: YMC Triart C18 100×19 mm, 5 μm) to afford 2-(4-cyclopropyl-6-methoxy-pyrimidin-5-yl)-9-[[4-[5-methyl-2-(trifluoromethyl)oxazol-4-yl]phenyl]methyl]-7-(2,2,2-trifluoroethyl)purin-8-imine (13.8 mg, 22.8 μmol, 44.8%) as a yellow solid.

¹H NMR (600 MHz, DMSO-d6) δ 0.80-0.84 (m, 2H), 0.99-1.03 (m, 2H), 1.67-1.71 (m, 1H), 2.61 (s, 3H), 3.83 (s, 3H), 4.78-5.04 (m, 2H), 5.07-5.24 (m, 2H), 7.04-7.12 (m, 1H), 7.49 (d, 2H), 7.62-7.67 (m, 2H), 8.30-8.38 (m, 1H), 8.64 (s, 1H).

LCMS(ESI): [M+H]+ m/z: calcd 605.21; found 605.2.

### Example T-141

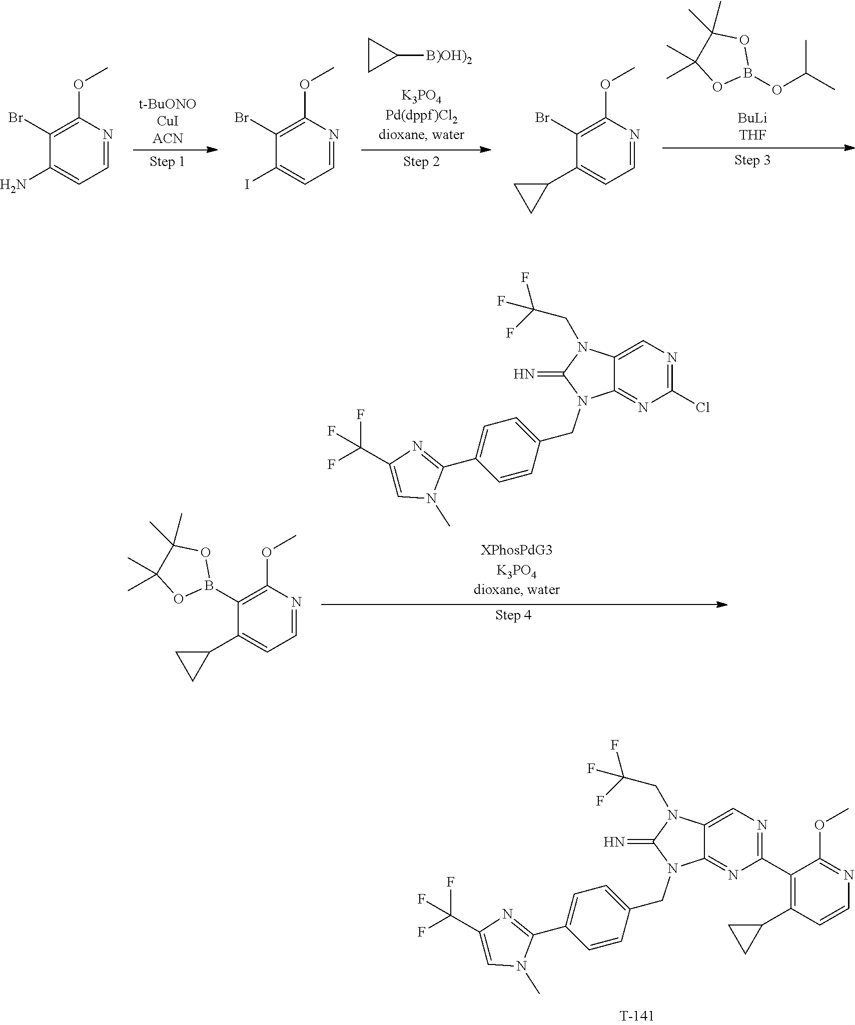

### Step 1: The synthesis of 3-bromo-4-iodo-2-methoxy-pyridine

3-Bromo-2-methoxy-pyridin-4-amine (2.90 g, 14.3 mmol) and copper(I) iodide (4.08 g, 21.4 mmol) were mixed in ACN (150 mL). To the resulting mixture tert-butyl nitrite (1.91 g, 18.6 mmol, 2.21 mL) was added dropwise at room temperature. The reaction mixture was stirred at 80° C. for 24 hr. The reaction mixture was cooled to room temperature and filtered. The filtrate was diluted with water (70 mL) and extracted with EtOAc (2×50 mL). The combined organic layers were washed with aqueous NH4OH solution (2×20 mL, 5% wt.), aqueous Na2S2O3 (10 mL, 5% wt.), aqueous NaHCO3 (10 mL, 5% wt.) and brine (20 mL), dried over anhydrous Na2SO4 and concentrated under reduce pressure to afford 3-bromo-4-iodo-2-methoxy-pyridine (1.8 g, 5.73 mmol, 4.15% yield) as a black solid which was used in the next steps without further purification.

¹H NMR (600 MHz, DMSO-d6) δ 3.88 (s, 3H), 7.51 (d, 1H), 7.83 (d, 1H).

GCMS: [M]+ m/z: calcd 312.86; found 313

### Step 2: The synthesis of 3-bromo-4-cyclopropyl-2-methoxy-pyridine

3-bromo-4-iodo-2-methoxy-pyridine (1.30 g, 4.14 mmol), cyclopropylboronic acid (427 mg, 4.97 mmol), potassium phosphate tribasic (2.64 g, 12.4 mmol) and bis(diphenylphosphino)ferrocene]dichloropalladium(II)-DCM (169 mg, 0.21 mmol) were mixed in a degassed mixture of dioxane and water (150 mL, 9:1) under and argon atmosphere. The mixture was stirred at 100° C. for 16 hr and an additional amount of cyclopropylboronic acid (427 mg, 4.97 mmol) was added. The reaction mixture was stirred at 100° C. for an additional 16 hr. The reaction mixture was cooled to room temperature and filtered through a short pad of SiO2. The filtrate was diluted with water (30 mL) and extracted with EtOAc (2×25 mL). The combined organic layers were washed with brine (15 mL), dried over anhydrous sodium sulfate, filtered and concentrated under reduced pressure. The residue was subjected to flash-column chromatography (SiO2, gradient elution: hexane—EtOAc) to afford 3-bromo-4-cyclopropyl-2-methoxy-pyridine (300 mg, 1.32 mmol, 31.8% yield) as a light-yellow solid.

¹H NMR (500 MHz, CDCl3) δ 0.70-0.76 (m, 2H), 1.07-1.13 (m, 2H), 2.24-2.31 (m, 1H), 3.99 (s, 3H), 6.31 (d, 1H), 7.91 (d, 1H).

### Step 3: The synthesis of 4-cyclopropyl-2-methoxy-3-(4,4,5,5-tetramethyl-1,3,2-dioxaborolan-2-yl)pyridine

Butyllithium (1.25 mmol, 500 μL, 2.5M in hexane) was added dropwise to a precooled to −78° C. mixture of 3-bromo-4-cyclopropyl-2-methoxy-pyridine (212 mg, 837 μmol) and 2-isopropoxy-4,4,5,5-tetramethyl-1,3,2-dioxaborolane (234 mg, 1.25 mmol) in THE (5 mL) under an argon atmosphere. The reaction mixture was stirred at −78° C. for 3 hr. The reaction mixture was allowed to warm to 5° C. and quenched with water (5.0 mL). The resulting mixture was extracted with EtOAc (5.0 mL). The organic layer was washed with brine (3.0 mL), dried over anhydrous sodium sulfate and filtered. The filtrate was concentrated under reduced pressure to afford 4-cyclopropyl-2-methoxy-3-(4,4,5,5-tetramethyl-1,3,2-dioxaborolan-2-yl)pyridine (250 mg, crude) as a light-yellow oil which was used in the next steps without further purification.

LCMS(ESI): [M+H]+ m/z: calcd 276.18; found 276.2.

### Step 4: The synthesis of 2-(4-cyclopropyl-2-methoxy-3-pyridyl)-9-[[4-[I-methyl-4-(trifluoromethyl)imidazol-2-yl]phenyl]methyl]-7-(2,2,2-trifluoroethyl)purin-8-imine

2-Chloro-9-[[4-[1-methyl-4-(trifluoromethyl)imidazol-2-yl]phenyl]methyl]-7-(2,2,2-trifluoroethyl)purin-8-imine (70.9 mg, 145 μmol), 4-cyclopropyl-2-methoxy-3-(4,4,5,5-tetramethyl-1,3,2-dioxaborolan-2-yl)pyridine (100 mg, crude), potassium phosphate tribasic anhydrous (52.1 mg, 245 μmol) were mixed in degassed dioxane (9.0 mL) and water (1.0 mL). XPhos Pd G3 (6.92 mg, 8.18 μmol) was added to the mixture. The reaction mixture was stirred at 95° C. for 12 hr. 4-cyclopropyl-2-methoxy-3-(4,4,5,5-tetramethyl-1,3,2-dioxaborolan-2-yl)pyridine (100 mg, crude) and XPhos Pd G3 (6.92 mg, 8.18 μmol) were added to the reaction mixture and the mixture was stirred at 95° C. for 12 hr. The reaction mixture was cooled to room temperature and diluted with water (5.0 mL). The resulting mixture was extracted with EtOAc (2×3.0 mL). The combined organic layers were washed with brine (3.0 mL), dried over anhydrous sodium sulfate and filtered. SiliaMetS® Dimercaptotriazine (50.0 mg) was added to the filtrate and the resulting mixture was stirred for 30 min. The mixture was filtered. The filtrate was concentrated under reduced pressure. The residue was subjected to HPLC (gradient elution: 20-30% water+FA (0.1% vol.)—ACN+FA (0.1% vol.); flow: 30 mL/min, column: XBridge, 100×19 mm, 5 μm) to afford 2-(4-cyclopropyl-2-methoxy-3-pyridyl)-9-[[4-[1-methyl-4-(trifluoromethyl)imidazol-2-yl]phenyl]methyl]-7-(2,2,2-trifluoroethyl)purin-8-imine (9.00 mg, 14.9 μmol, 18.3% yield) as a brown solid.

¹H NMR (600 MHz, DMSO-d6) δ 0.65-0.70 (m, 2H), 0.75-0.80 (m, 2H), 1.41-1.48 (m, 1H), 3.70-3.75 (m, 6H), 4.76-5.02 (m, 2H), 5.06-5.25 (m, 2H), 6.48 (d, 1H), 6.97-7.07 (m, 1H), 7.46 (d, 2H), 7.61-7.67 (m, 2H), 7.90 (s, 1H), 8.02 (d, 1H), 8.27-8.34 (m, 1H).

LCMS(ESI): [M+H]+ m/z: calcd 603.24; found 603.2.

### Example T-125

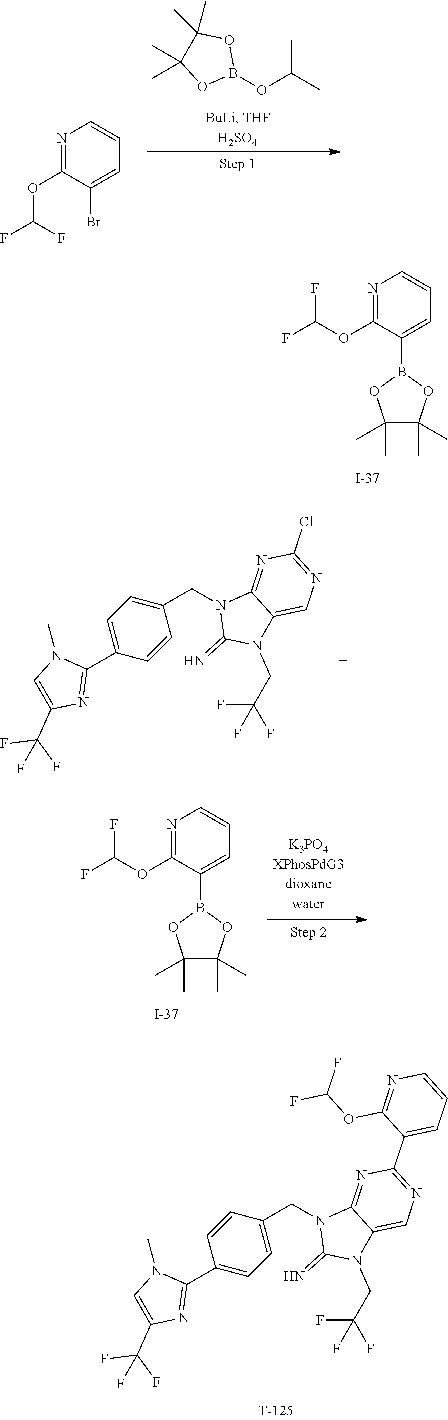

### Step 1: The synthesis of 2-(difluoromethoxy)-3-(4,4,5,5-tetramethyl-1,3,2-dioxaborolan-2-yl)pyridine

Step 1 is described in Intermediate 37.

### Step 2: The synthesis of 2-[2-(difluoromethoxy)-3-pyridyl]-9-[[4-[I-methyl-4-(trifluoromethyl)imidazol-2-yl]phenyl]methyl]-7-(2,2,2-trifluoroethyl)purin-8-imine

2-Chloro-9-[[4-[1-methyl-4-(trifluoromethyl)imidazol-2-yl]phenyl]methyl]-7-(2,2,2-trifluoroethyl)purin-8-imine (50.0 mg, 102 μmol), 2-(difluoromethoxy)-3-(4,4,5,5-tetramethyl-1,3,2-dioxaborolan-2-yl)pyridine (55.3 mg, 204 μmol), potassium phosphate tribasic anhydrous (65.0 mg, 306 μmol) and XPhos Pd G3 (6.05 mg, 7.15 μmol) were mixed in degassed mixture of dioxane (4.0 mL) and water (0.4 mL) under an argon atmosphere. The reaction mixture was stirred at 85° C. for 12 hr. The reaction mixture was cooled to room temperature. SiliaMetS® Dimercaptotriazine (100 mg) were added to the reaction mixture. The resulting mixture was stirred at room temperature for 3 hr. The mixture was diluted with MTBE (5.0 mL) and filtered through a pad of silica gel. The filtrate was concentrated under reduced pressure. The residue was subjected to HPLC purification (gradient elution: 30-45% ACN+FA (0.1% vol.); flow: 30 mL/min, column: SunFire C18, 100×19 mm, 5 μm) to afford 2-[2-(difluoromethoxy)-3-pyridyl]-9-[[4-[1-methyl-4-(trifluoromethyl)imidazol-2-yl]phenyl]methyl]-7-(2,2,2-trifluoroethyl)purin-8-imine (13.9 mg, 23.2 μmol, 22.8% yield) as a yellow solid.

¹H NMR (600 MHz, DMSO-d6) δ 3.75 (s, 3H), 4.73-5.30 (m, 4H), 7.01-7.19 (m, 1H), 7.38-7.43 (m, 1H), 7.59 (d, 2H), 7.68 (d, 2H), 7.79 (t, 1H, CHF2), 7.91 (s, 1H), 8.25 (d, 1H), 8.31-8.40 (m, 2H).

LCMS(ESI): [M+H]+ m/z: calcd 599.18; found 599.2.

### Example T-142

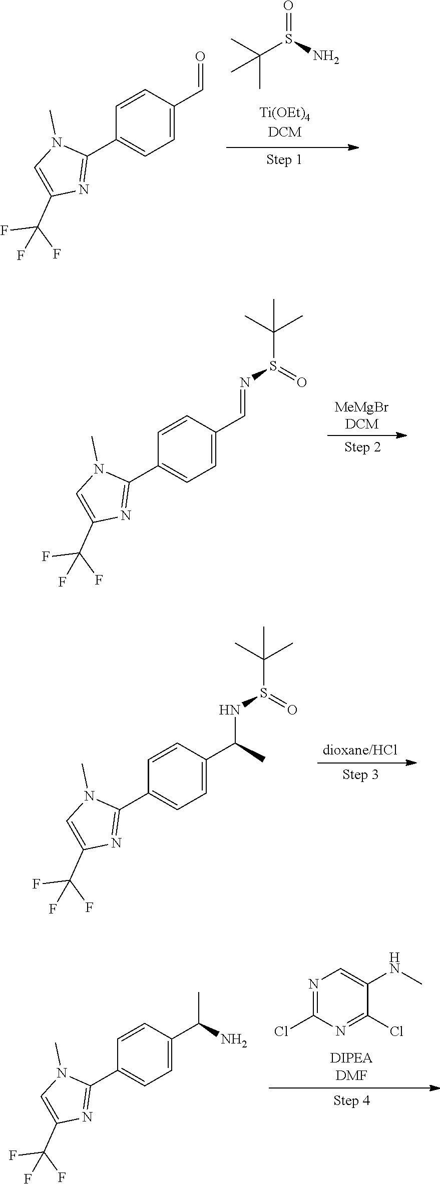

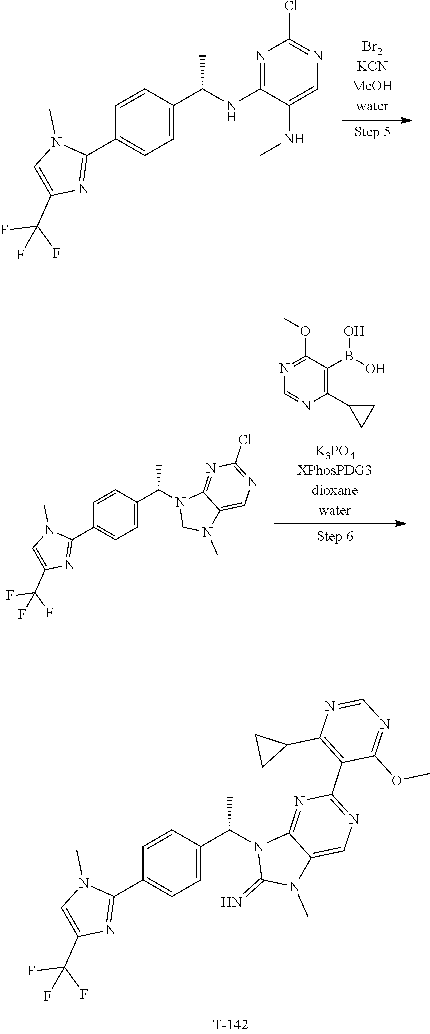 [SMILES: CNc1cnc(Cl)nc1N[C@@H](C)c1ccc(-c2nc(C(F)(F)F)cn2C)cc1 ; COc1ncnc(C2CC2)c1B(O)O ; C[C@@H](c1ccc(-c2nc(C(F)(F)F)cn2C)cc1)N1CN(C)c2cnc(Cl)nc21 ; COc1ncnc(C2CC2)c1-c1ncc2c(n1)n([C@@H](C)c1ccc(-c3nc(C(F)(F)F)cn3C)cc1)c(=N)n2C]

### Step 1: The synthesis of (R)-2-methyl-N-(4-(1-methyl-4-(trifluoromethyl)-1H-imidazol-2-yl)benzylidene)propane-2-sulfinamide

Titanium (IV) ethoxide (2.24 g, 9.83 mmol, 2.06 mL) was added to a solution of 4-[1-methyl-4-(trifluoromethyl)imidazol-2-yl]benzaldehyde (500 mg, 1.97 mmol) in DCM (5.79 mL). The reaction mixture was stirred at room temperature for 20 min. (R)-2-methylpropane-2-sulfinamide (238 mg, 1.97 mmol) was added to the reaction mixture. The resulting mixture was stirred at room temperature for 18 hr. The reaction mixture was diluted with DCM (30 mL) and quenched with a solution of aqueous NaHCO3 (20 mL). The solids were filtered out. The filtrate was extracted with DCM (2×20 mL). The combined organic layers were dried over anhydrous sodium sulfate and concentrated under reduced pressure to afford (R)-2-methyl-N-(4-(1-methyl-4-(trifluoromethyl)-1H-imidazol-2-yl)benzylidene)propane-2-sulfinamide (680 mg, 1.90 mmol, 96.7% yield) as a yellow solid which was used in the next steps without further purification.

¹H NMR (400 MHz, CDCl3) δ 1.27 (s, 9H), 3.81 (s, 3H), 7.34 (s, 1H), 7.76 (d, 2H), 7.94 (d, 2H), 8.61 (s, 1H).

LCMS(ESI): [M+H]+ m/z: calcd 358.15; found 358.0.

### Step 2: The synthesis of (R)-2-methyl-N-[(1S)-1-[4-[I-methyl-4-(trifluoromethyl)imidazol-2-yl]phenyl]ethyl]propane-2-sulfinamide

Methylmagnesium bromide (2.38 g, 7.00 mmol, 35% wt. in MeTHF) was added to a precooled to −30° C. solution of (R)-2-methyl-N-(4-(1-methyl-4-(trifluoromethyl)-1H-imidazol-2-yl)benzylidene)propane-2-sulfinamide (500 mg, 1.40 mmol) in DCM (100 mL). The reaction mixture was stirred at −30° C. for 1 hr. The reaction mixture was allowed to warm up to room temperature, quenched by addition of acetone (10 mL) and washed with water (2×20 mL). The organic layer was dried over anhydrous sodium sulfate and concentrated under reduced pressure to afford (R)-2-methyl-N-[(1S)-1-[4-[1-methyl-4-(trifluoromethyl)imidazol-2-yl]phenyl]ethyl]propane-2-sulfinamide (530 mg, crude) as a yellow oil which was used in the next steps without further purification.

LCMS(ESI): [M+H]+ m/z: calcd 374.19; found 374.0.

### Step 3: The synthesis of (1S)-1-[4-[I-methyl-4-(trifluoromethyl)imidazol-2-yl]phenyl]ethanamine

(R)-2-methyl-N-[(1S)-1-[4-[1-methyl-4-(trifluoromethyl)imidazol-2-yl]phenyl]ethyl]propane-2-sulfinamide (530 mg, crude) was suspended in 4M solution of hydrogen chloride in dioxane (5 mL). The reaction mixture was stirred at room temperature for 18 hr. The reaction mixture was concentrated under reduced pressure to afford (1S)-1-[4-[1-methyl-4-(trifluoromethyl)imidazol-2-yl]phenyl]ethanamine (440 mg, crude, HCl) as an off-white solid which was used in the next steps without further purification.

¹H NMR (500 MHz, DMSO-d6) δ 1.53 (d, 3H), 3.78 (s, 3H), 7.66 (d, 2H), 7.76 (d, 2H), 7.96 (s, 1H), 8.65 (br, 3H).

LCMS(ESI): [M+H]+ m/z: calcd 270.15; found 270.0.

### Step 4: The synthesis of 2-chloro-N5-methyl-N4-[(1S)-1-[4-[1-methyl-4-(trifluoromethyl)imidazol-2-yl]phenyl]ethyl]pyrimidine-4,5-diamine

2,4-Dichloro-N-methyl-pyrimidin-5-amine (629 mg, 3.53 mmol) and DIPEA (685 mg, 5.30 mmol, 923 μL) were added to a solution of (1S)-1-[4-[1-methyl-4-(trifluoromethyl)imidazol-2-yl]phenyl]ethanamine (540 mg, crude, HCl) in DMF (10 mL). The reaction mixture was stirred at 100° C. for 18 hr. The reaction mixture was cooled to room temperature, diluted with water (50 mL) and extracted with EtOAc (3×50 mL). The combined organic layers were washed with brine (50 mL) and concentrated under reduced pressure. The residue was subjected to HPLC (gradient elution: 40-65% water—ACN, flow: 30 mL/min, column: SunFire C18 100×19 mm, 5 μm) to afford 2-chloro-N5-methyl-N4-[(1S)-1-[4-[1-methyl-4-(trifluoromethyl)imidazol-2-yl]phenyl]ethyl]pyrimidine-4,5-diamine (200 mg, 487 μmol, 27.6% yield) as a brown solid.

LCMS(ESI): [M+H]+ m/z: calcd 411.16; found 411.2.

### Step 5: The synthesis of 2-chloro-7-methyl-9-[(1S)-1-[4-[-methyl-4-(trifluoromethyl)imidazol-2-yl]phenyl]ethyl]purin-8-imine

A solution of potassium cyanide (499 mg, 7.67 mmol) in water (5.0 mL) was added dropwise to a solution of Br2 (1.23 g, 7.67 mmol) in MeOH (50 mL) at 0° C. The resulting mixture was stirred at 0° C. for 15 min. A solution of 2-chloro-N5-methyl-N4-[(1S)-1-[4-[1-methyl-4-(trifluoromethyl)imidazol-2-yl]phenyl]ethyl]pyrimidine-4,5-diamine (210 mg, 511 mol) in MeOH (2.0 mL) was added to the mixture. The reaction mixture was stirred at room temperature for 100 hr. The reaction mixture was diluted with water (100 mL) and extracted with EtOAc (3×50 mL). The combined organic layers were washed with brine (50 mL) dried over anhydrous sodium sulfate and concentrated under reduced pressure to afford 2-chloro-7-methyl-9-[(1S)-1-[4-[1-methyl-4-(trifluoromethyl)imidazol-2-yl]phenyl]ethyl]purin-8-imine (210 mg, 482 μmol, 94.3% yield) as a white solid which was used in the next step without further purification.

LCMS(ESI): [M+H]+ m/z: calcd 436.15; found 436.0.

### Step 6: The synthesis of 2-(4-cyclopropyl-6-methoxy-pyrimidin-5-yl)-7-methyl-9-[(1S)-1-[4-[1-methyl-4-(trifluoromethyl)imidazol-2-yl]phenyl]ethyl]purin-8-imine

2-Chloro-7-methyl-9-[(1S)-1-[4-[1-methyl-4-(trifluoromethyl)imidazol-2-yl]phenyl]ethyl]purin-8-imine (210 mg, 482 μmol) was dissolved in dioxane (7.0 mL) and water (500 μL). The resulting mixture was degassed twice. (4-cyclopropyl-6-methoxy-pyrimidin-5-yl)boronic acid (140 mg, 723 μmol), potassium phosphate tribasic (307 mg, 1.45 mmol) and XPhos Pd G3 (40.8 mg, 48.2 μmol) were added to the mixture. The reaction mixture was stirred at 85° C. for 18 hr. The reaction mixture was cooled to room temperature and diluted with methanol (5.0 mL). SiliaMetS® Dimercaptotriazine (100 mg) was added to the resulting organic layer. The resulting mixture was stirred for 5 hr and filtered. The filtrate was concentrated under reduced pressure. The residue was subjected to HPLC (gradient elution: 30-55% water—ACN, +0.1% vol. of 25% aq. NH3, flow: 30 mL/min, column: XBridge C18 100×19 mm, 5 μm) to afford 2-(4-cyclopropyl-6-methoxy-pyrimidin-5-yl)-7-methyl-9-[(1S)-1-[4-[1-methyl-4-(trifluoromethyl)imidazol-2-yl]phenyl]ethyl]purin-8-imine (41.0 mg, 74.6 μmol, 15.5% yield) as a yellow solid.

¹H NMR (600 MHz, DMSO-d6) δ 0.76-0.86 (m, 2H), 0.95-1.03 (m, 2H), 1.69-1.75 (m, 1H), 1.96 (d, 3H), 3.33-3.39 (m, 3H), 3.75 (s, 3H), 3.82 (s, 3H), 5.80-5.93 (m, 1H), 6.45-6.57 (m, 1H), 7.55 (d, 2H), 7.62-7.69 (m, 2H), 7.91 (s, 1H), 8.18-8.27 (m, 1H), 8.61 (s, 1H).

LCMS(ESI): [M+H]+ m/z: calcd 550.26; found 550.2.

### Example T-138

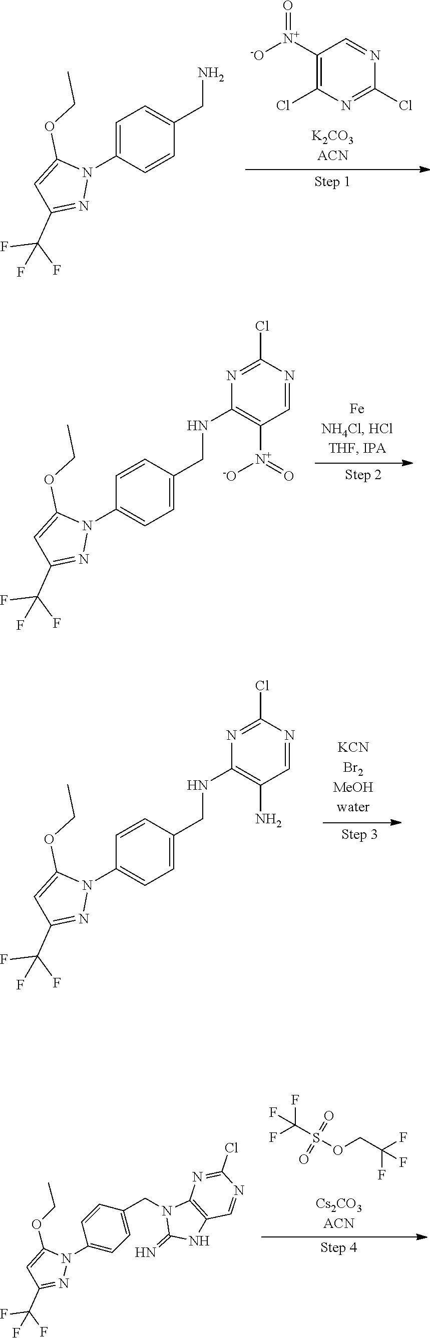

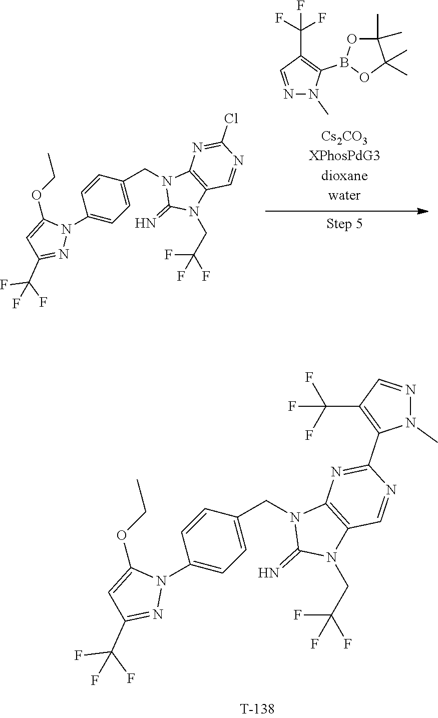 [SMILES: Cn1ncc(C(F)(F)F)c1B1OC(C)(C)C(C)(C)O1 ; CCOc1cc(C(F)(F)F)nn1-c1ccc(Cn2c(=N)n(CC(F)(F)F)c3cnc(Cl)nc32)cc1 ; CCOc1cc(C(F)(F)F)nn1-c1ccc(Cn2c(=N)n(CC(F)(F)F)c3cnc(-c4c(C(F)(F)F)cnn4C)nc32)cc1]

### Step 1: The synthesis of 2-chloro-N-[[4-[5-ethoxy-3-(trifluoromethyl)pyrazol-1-yl]phenyl]methyl]-5-nitro-pyrimidin-4-amine

2,4-dichloro-5-nitro-pyrimidine (548 mg, 2.83 mmol) was added to a 0° C. stirred mixture of [4-[5-ethoxy-3-(trifluoromethyl)pyrazol-1-yl]phenyl]methanamine (806 mg, 2.83 mmol) and potassium carbonate (781 mg, 5.65 mmol) in ACN (30 mL). The reaction mixture was allowed to warm and stirred at room temperature for 16 hr. The reaction mixture was concentrated under reduced pressure. The resulting residue was diluted with water (30 mL) and extracted with EtOAc (40 mL). The organic layer was washed with brine (30 mL), dried over anhydrous sodium sulfate and concentrated under reduced pressure to afford 2-chloro-N-[[4-[5-ethoxy-3-(trifluoromethyl)pyrazol-1-yl]phenyl]methyl]-5-nitro-pyrimidin-4-amine (983 mg, 2.22 mmol, 78.6% yield) as a brown solid which was used in the next steps without further purification.

LCMS(ESI): [M+H]+ m/z: calcd 443.09; found 443.0.

### Step 2: The synthesis of 2-chloro-N4-[[4-[5-ethoxy-3-(trifluoromethyl)pyrazol-1-yl]phenyl]methyl]pyrimidine-4,5-diamine

Hydrochloric acid (3.00 mL, 36% wt. aqueous soln.) and iron powder (868 mg, 15.5 mmol) were added to a stirred mixture of 2-chloro-N-[[4-[5-ethoxy-3-(trifluoromethyl)pyrazol-1-yl]phenyl]methyl]-5-nitro-pyrimidin-4-amine (983 mg, 2.22 mmol) and ammonium chloride (1.19 g, 22.2 mmol) in THE (20 mL) and IPA (20 mL). The reaction mixture was stirred at 45° C. for 18 hr. The reaction mixture was cooled to room temperature and filtered. The filter cake was washed with IPA (5.0 mL). The combined filtrate was concentrated under reduced pressure. The residue was diluted with an aqueous solution of potassium carbonate (40 mL, 10% wt.) and extracted with EtOAc (60 mL). The organic layer was washed with brine (30 mL), dried over anhydrous sodium sulfate and concentrated under reduced pressure to afford 2-chloro-N4-[[4-[5-ethoxy-3-(trifluoromethyl)pyrazol-1-yl]phenyl]methyl]pyrimidine-4,5-diamine (760 mg, 1.84 mmol, 82.9% yield) as a brown solid which was used in the next steps without further purification.

LCMS(ESI): [M+H]+ m/z: calcd 413.13; found 413.2.

### Step 3: The synthesis of 2-chloro-9-[[4-[5-ethoxy-3-(trifluoromethyl)pyrazol-1-yl]phenyl]methyl]-7H-purin-8-imine

A solution of potassium cyanide (1.12 g, 17.2 mmol) in water (2.0 mL) was added to a stirred solution of bromine (2.75 g, 17.2 mmol) in MeOH (25 mL) at room temperature. The resulting mixture was stirred at room temperature for 15 min. A solution of 2-chloro-N4-[[4-[5-ethoxy-3-(trifluoromethyl)pyrazol-1-yl]phenyl]methyl]pyrimidine-4,5-diamine (710 mg, 1.72 mmol) in MeOH (2.0 mL) was added to the mixture. The reaction mixture was stirred at room temperature for 16 hr. The reaction mixture was concentrated under reduced pressure. The resulting residue was diluted with EtOAc (80 mL) and washed with an aqueous solution of potassium carbonate (50 mL, 10% wt.). The organic layer was dried over anhydrous sodium sulfate and filtered. The filtrate was concentrated under reduced pressure. The residue was subjected to HPLC (0-5 min, 35-65% water—ACN; flow: 30 mL/min, column: Chromatorex 18 SMB100-5T, 100×19 mm, 5 μm) to afford 2-chloro-9-[[4-[5-ethoxy-3-(trifluoromethyl)pyrazol-1-yl]phenyl]methyl]-7H-purin-8-imine (680 mg, 1.55 mmol, 90.3% yield) as a yellow solid.

LCMS(ESI): [M+H]+ m/z: calcd 438.12; found 438.2.

### Step 4: The synthesis of 2-chloro-9-[[4-[5-ethoxy-3-(trifluoromethyl)pyrazol-1-yl]phenyl]methyl]-7-(2,2,2-trifluoroethyl)purin-8-imine

2,2,2-trifluoroethyl trifluoromethanesulfonate (83.5 mg, 360 μmol) was added to a stirred mixture of 2-chloro-9-[[4-[5-ethoxy-3-(trifluoromethyl)pyrazol-1-yl]phenyl]methyl]-7H-purin-8-imine (105 mg, 240 μmol) and cesium carbonate (156 mg, 480 μmol) in ACN (3.0 mL). The reaction mixture was stirred at 65° C. for 14 hr. The reaction mixture was cooled to room temperature and concentrated under reduced pressure. The residue was diluted with water (30 mL) and extracted with EtOAc (40 mL). The organic layer was washed with brine (30 mL), dried over anhydrous sodium sulfate and concentrated under reduced pressure. The residue was subjected to HPLC (gradient elution: 50-100% water+FA (0.1% vol.)—MeOH+FA (0.1% vol.); flow: 30 mL/min, column: Chromatorex 18 SMB100-5T, 100×19 mm, 5 μm) to afford 2-chloro-9-[[4-[5-ethoxy-3-(trifluoromethyl)pyrazol-1-yl]phenyl]methyl]-7-(2,2,2-trifluoroethyl)purin-8-imine (25.0 mg, 48.1 μmol, 20.1% yield) as a yellow solid.

¹H NMR (400 MHz, DMSO-d6) δ 1.34 (t, 3H), 4.26 (q, 2H), 4.72-5.00 (m, 2H), 5.02-5.23 (m, 2H), 6.44 (s, 1H), 7.16-7.28 (m, 1H), 7.42-7.51 (m, 2H), 7.59-7.68 (m, 2H), 8.02-8.16 (m, 1H).

LCMS(ESI): [M+H]+ m/z: calcd 520.13; found 520.4.

### Step 5: The synthesis of 9-[[4-[5-ethoxy-3-(trifluoromethyl)pyrazol-1-yl]phenyl]methyl]-2-[2-methyl-4-(trifluoromethyl)pyrazol-3-yl]-7-(2,2,2-trifluoroethyl)purin-8-imine

1-methyl-5-(4,4,5,5-tetramethyl-1,3,2-dioxaborolan-2-yl)-4-(trifluoromethyl)pyrazole (26.6 mg, 96.2 μmol), cesium carbonate (47.0 mg, 144 μmol) and XPhosPdG3 (2.04 mg, 2.40 mol) were added to a solution of 2-chloro-9-[[4-[5-ethoxy-3-(trifluoromethyl)pyrazol-1-yl]phenyl]methyl]-7-(2,2,2-trifluoroethyl)purin-8-imine (25.0 mg, 48.1 μmol) in a degassed mixture of dioxane (2.0 mL) and water (500 μL) under an argon atmosphere. The reaction mixture was stirred at 90° C. for 16 hr. The reaction mixture was cooled to room temperature and concentrated under reduced pressure. The residue was diluted with EtOAc (20 mL) and washed with brine (10 mL). The organic layer was dried over anhydrous sodium sulfate and concentrated under reduced pressure. The residue was subjected to HPLC (gradient elution: 40-85% water -ACN, +0.1% vol. of 25% aq. NH3, flow: 30 mL/min, column: YMC Triart C18 100×20 mm, 5 μm) to afford 9-[[4-[5-ethoxy-3-(trifluoromethyl)pyrazol-1-yl]phenyl]methyl]-2-[2-methyl-4-(trifluoromethyl)pyrazol-3-yl]-7-(2,2,2-trifluoroethyl)purin-8-imine (14.8 mg, 23.4 μmol, 48.6% yield) as a light-yellow solid.

¹H NMR (600 MHz, DMSO-d6) δ 1.32 (t, 3H), 3.96-4.02 (m, 3H), 4.25 (q, 2H), 4.79-5.03 (m, 2H), 5.07-5.24 (m, 2H), 6.42 (s, 1H), 7.16-7.26 (m, 1H), 7.47-7.55 (m, 2H), 7.58-7.63 (m, 2H), 7.89 (s, 1H), 8.35-8.42 (m, 1H).

LCMS(ESI): [M+H]+ m/z: calcd 634.20; found 634.2.

### Example T-134

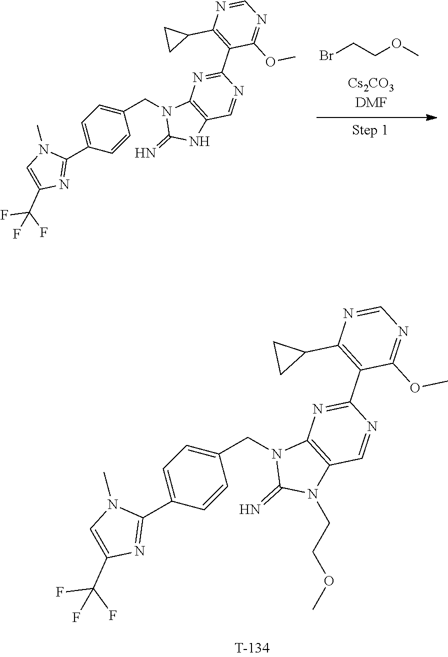

### Step 1: The synthesis of 2-(4-cyclopropyl-6-methoxy-pyrimidin-5-yl)-7-(2-methoxyethyl)-9-[[4-[1-methyl-4-(trifluoromethyl)imidazol-2-yl]phenyl]methyl]purin-8-imine

2-(4-cyclopropyl-6-methoxy-pyrimidin-5-yl)-9-[[4-[1-methyl-4-(trifluoromethyl)imidazol-2-yl]phenyl]methyl]-7H-purin-8-imine (17.0 mg, 32.6 μmol), 1-bromo-2-methoxy-ethane (9.06 mg, 65.2 μmol) and cesium carbonate (21.2 mg, 65.2 μmol) were mixed in DMF (1.0 mL). The reaction mixture was stirred at 70° C. for 12 hr. The mixture was cooled to room temperature and filtered. The filtrate was subjected to HPLC purification (gradient elution: 20-40% water—ACN, +0.1% vol. of 25% aq. NH3, flow: 30 mL/min, column: YMC-ACTUS TRIART C18 100×20 mm, 5 μm) to afford 2-(4-cyclopropyl-6-methoxy-pyrimidin-5-yl)-7-(2-methoxyethyl)-9-[[4-[1-methyl-4-(trifluoromethyl)imidazol-2-yl]phenyl]methyl]purin-8-imine (2.30 mg, 3.97 μmol, 12.2% yield) as an off-white solid.

¹H NMR (600 MHz, DMSO-d6) δ 0.79-0.84 (m, 2H), 0.97-1.01 (m, 2H), 1.65-1.71 (m, 1H), 3.26 (s, 3H), 3.57-3.67 (m, 2H), 3.73 (s, 3H), 3.82 (s, 3H), 4.02-4.09 (m, 2H), 5.07-5.21 (m, 2H), 6.60-6.66 (m, 1H), 7.48 (d, 2H), 7.62-7.68 (m, 2H), 7.90 (s, 1H), 8.18-8.26 (m, 1H), 8.61 (s, 1H).

LCMS(ESI): [M+H]+ m/z: calcd 580.27; found 580.2.

### Example T-119

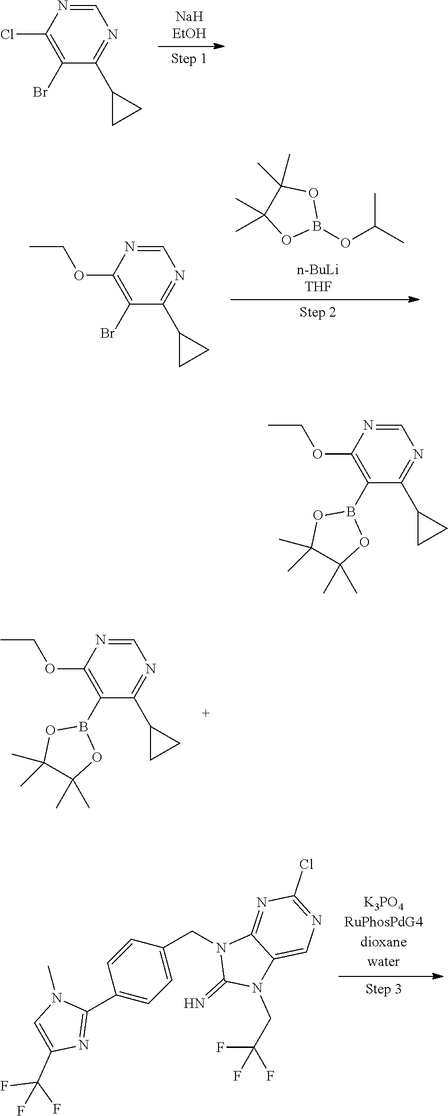 [SMILES: Clc1ncnc(C2CC2)c1Br ; CC(C)OB1OC(C)(C)C(C)(C)O1 ; CCOc1ncnc(C2CC2)c1Br ; CCOc1ncnc(C2CC2)c1B1OC(C)(C)C(C)(C)O1 ; CCOc1ncnc(C2CC2)c1B1OC(C)(C)C(C)(C)O1 ; Cn1cc(C(F)(F)F)nc1-c1ccc(Cn2c(=N)n(CC(F)(F)F)c3cnc(Cl)nc32)cc1]

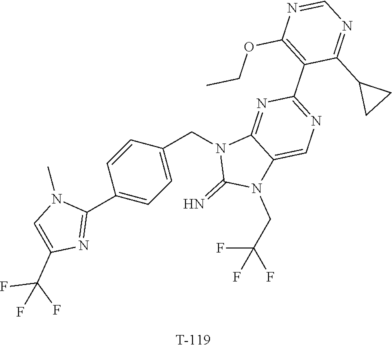 [SMILES: CCOc1ncnc(C2CC2)c1-c1ncc2c(n1)n(Cc1ccc(-c3nc(C(F)(F)F)cn3C)cc1)c(=N)n2CC(F)(F)F]

### Step 1: The synthesis of 5-bromo-4-cyclopropyl-6-ethoxy-pyrimidine

Sodium hydride (295 mg, 12.9 mmol, 60% dispersion in mineral oil) was added portionwise to stirred EtOH (150 mL) under an argon atmosphere. The resulting mixture was stirred at room temperature for 20 min then cooled to −20° C. 5-bromo-4-chloro-6-cyclopropyl-pyrimidine (3.00 g, 12.9 mmol) was added to the solution at −20° C. The reaction mixture was stirred at room temperature for 16 hr. The reaction mixture was concentrated under reduced pressure. The residue was poured into a mixture of ice and water (100 mL). The solid precipitate formed was collected by filtration and dissolved in EtOAc (120 mL). The resulting organic layer was washed with brine (2×30 mL), dried over anhydrous sodium sulfate and concentrated under reduced pressure to afford 5-bromo-4-cyclopropyl-6-ethoxy-pyrimidine (2.80 g, 11.5 mmol, 89.6% yield) as a light-yellow solid which was used in the next step without further purification. 1HNMR (CDCl3, 500 MHz) δ 1.05-1.10 (m, 2H), 1.14-1.19 (m, 2H), 1.44 (t, 3H), 2.49-2.51 (m, 1H), 4.47 (q, 2H), 8.41 (s, 1H).

LCMS(ESI): [M+H]+ m/z: calcd 243.02; found 243.0.

### Step 2: The synthesis of 4-cyclopropyl-6-ethoxy-5-(4,4,5,5-tetramethyl-1,3,2-dioxaborolan-2-yl)pyrimidine

5-bromo-4-cyclopropyl-6-ethoxy-pyrimidine (1.80 g, 7.40 mmol) and 2-isopropoxy-4,4,5,5-tetramethyl-1,3,2-dioxaborolane (2.07 g, 11.1 mmol) were mixed in THE (100 mL) under argon atmosphere. n-Butyllithium (13.3 mmol, 5.32 mL, 2.5 M in hexane) was added dropwise to the precooled to −78° C. solution. The reaction mixture was stirred at −70° C. for 3 hr. The reaction mixture was allowed to warm to room temperature, quenched by dropwise addition of a saturated aqueous NH4Cl solution (20 mL) and extracted with EtOAc (50 mL). The organic layer was separated, washed with brine (2×20 mL), dried over anhydrous sodium sulfate and concentrated under reduced pressure. The residue was subjected to flash column chromatography (SiO2, gradient elution: hexane—MTBE) to afford 4-cyclopropyl-6-ethoxy-5-(4,4,5,5-tetramethyl-1,3,2-dioxaborolan-2-yl)pyrimidine (1.36 g, 4.70 mmol, 63.9% yield) as a light-yellow solid. 1HNMR (CDCl3, 400 MHz) δ 0.96-1.01 (m, 2H), 1.16-1.21 (m, 2H), 1.34-1.43 (m, 15H), 1.99-2.07 (m, 1H), 4.36 (q, 2H), 8.55 (s, 1H).

LCMS(ESI): [M+H]+ m/z: calcd 291.19; found 291.0.

### Step 3: The synthesis of 2-(4-cyclopropyl-6-ethoxy-pyrimidin-5-yl)-9-[[4-[I-methyl-4-(trifluoromethyl)imidazol-2-yl]phenyl]methyl]-7-(2,2,2-trifluoroethyl)purin-8-imine

2-chloro-9-[[4-[1-methyl-4-(trifluoromethyl)imidazol-2-yl]phenyl]methyl]-7-(2,2,2-trifluoroethyl)purin-8-imine (400 mg, 817 μmol), 4-cyclopropyl-6-ethoxy-5-(4,4,5,5-tetramethyl-1,3,2-dioxaborolan-2-yl)pyrimidine (474 mg, 1.63 mmol), potassium phosphate tribasic anhydrous (520 mg, 2.45 mmol) and RuPhos Pd G4 (34.7 mg, 40.8 μmol) were mixed in a degassed mixture of dioxane (14 mL) and water (2.0 mL). The resulting mixture was degassed. The reaction mixture was stirred at 80° C. for 16 hr. The reaction mixture was cooled to room temperature and concentrated under reduced pressure. The residue was diluted with EtOAc (40 mL) and washed with water (15 mL). The organic layer was separated, washed with brine (2×15 mL), dried over anhydrous sodium sulfate and filtered. SiliaMetS® Dimercaptotriazine (300 mg) was added to the filtrate and the resulting mixture was stirred for 1 hr. The resulting mixture was filtered. The filtrate was subjected to HPLC purification (gradient elution: 0-60% water+FA (0.1% vol.)—ACN+FA (0.1% vol.); flow: 30 mL/min, column: Waters SunFire C18, 100×19 mm, 5 μm) to afford 2-(4-cyclopropyl-6-ethoxy-pyrimidin-5-yl)-9-[[4-[1-methyl-4-(trifluoromethyl)imidazol-2-yl]phenyl]methyl]-7-(2,2,2-trifluoroethyl)purin-8-imine (7.00 mg, 11.3 μmol, 1.39% yield) as a light-yellow solid.

1HNMR (DMSO-d6, 600 MHz) δ 0.79-0.85 (m, 2H), 0.97-1.01 (m, 2H), 1.11 (t, 3H), 1.63-1.70 (m, 1H), 3.73 (s, 3H), 4.03 (q, 2H), 4.77-5.04 (m, 2H), 5.07-5.26 (m, 2H), 7.01-7.12 (m, 1H), 7.45 (d, 2H), 7.62-7.69 (m, 2H), 7.90 (s, 1H), 8.30-8.39 (m, 1H), 8.59 (s, 1H).

LCMS(ESI): [M+H]+ m/z: calcd 618.25; found 618.0.

### Example T-129 and T-148

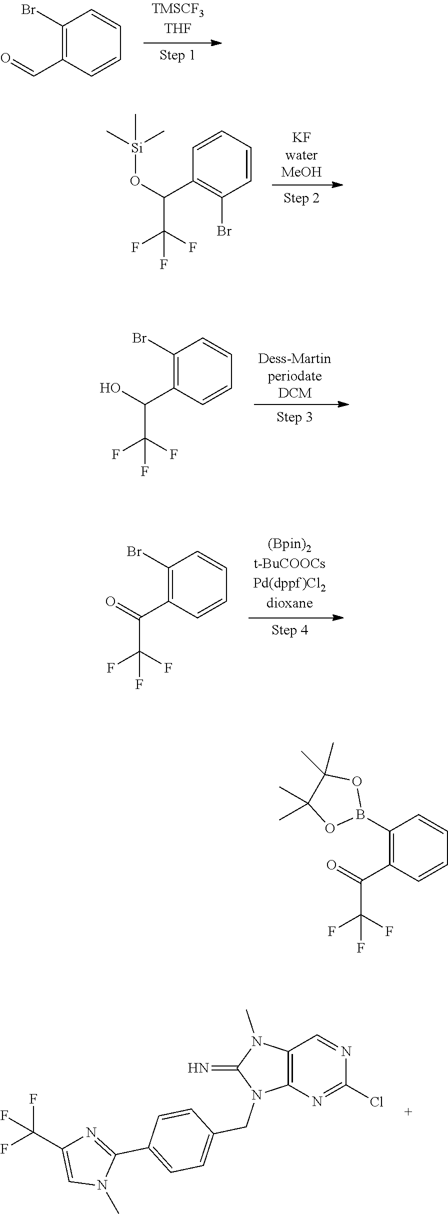 [SMILES: O=Cc1ccccc1Br ; C[Si](C)(C)OC(c1ccccc1Br)C(F)(F)F ; OC(c1ccccc1Br)C(F)(F)F ; O=C(c1ccccc1Br)C(F)(F)F ; CC1(C)OB(c2ccccc2C(=O)C(F)(F)F)OC1(C)C ; Cn1cc(C(F)(F)F)nc1-c1ccc(Cn2c(=N)n(C)c3cnc(Cl)nc32)cc1]

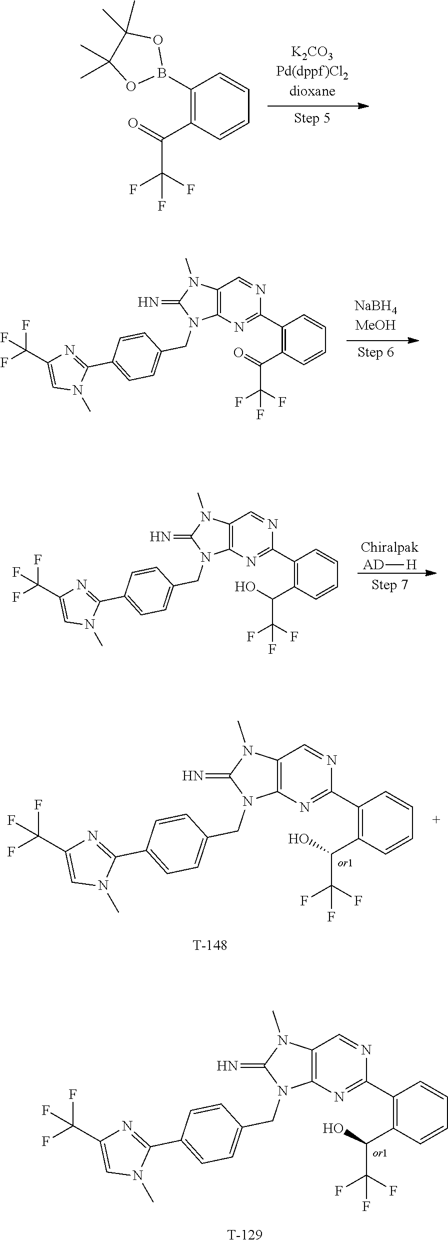

### Step 1: The synthesis of [1-(2-bromophenyl)-2,2,2-trifluoro-ethoxy]-trimethyl-silane

TBAF (424 mg, 1.62 mmol, 1.62 mL, 1M in THF) was added dropwise to a solution of 2-bromobenzaldehyde (10.0 g, 54.1 mmol) and trimethyl(trifluoromethyl)silane (11.5 g, 81.1 mmol) in THE (200 mL) at 0° C. The reaction mixture was at room temperature stirred for 12 hr. The mixture was concentrated under reduced pressure. The residue was dissolved in dichloromethane (200 mL) and washed with 2N aqueous hydrochloric acid (4×100 mL). The organic layer was separated, washed with aqueous Na2CO3 (50 mL, 10% wt. aqueous solution), dried over anhydrous MgSO4, filtered and concentrated under reduced pressure to afford [1-(2-bromophenyl)-2,2,2-trifluoro-ethoxy]-trimethyl-silane (11.2 g, 34.2 mmol, 63.2% yield) as a colorless oil which was used in the next steps without further purification.

¹H NMR (500 MHz, CDCl3) δ 0.14 (s, 9H), 5.55 (q, 1H), 7.26 (t, 1H), 7.39 (t, 1H), 7.59 (d, 1H), 7.71 (d, 1H).

### Step 2: Synthesis of 1-(2-bromophenyl)-2,2,2-trifluoro-ethanol

The solution of potassium fluoride (26.2 g, 451 mmol) in water (20 mL) was added to a solution of [1-(2-bromophenyl)-2,2,2-trifluoro-ethoxy]-trimethyl-silane (14.7 g, 45.1 mmol) in MeOH (50 mL). The reaction mixture was stirred at room temperature for 12 hr. The reaction mixture was diluted with water (200 mL) and extracted with DCM (2×200 mL). The combined organic layers were washed water (2×50 mL), dried over anhydrous sodium sulfate and concentrated under reduced pressure to afford 1-(2-bromophenyl)-2,2,2-trifluoro-ethanol (8.30 g, 32.5 mmol, 72.0% yield) as a colorless oil which was used in the next steps without further purification.

¹H NMR (400 MHz, CDCl3) δ 2.93-3.13 (br., 1H), 5.64 (q, 1H), 7.27 (t, 1H), 7.42 (t, 1H), 7.61 (d, 1H), 7.70 (d, 1H).

HRMS: [M]+ m/z: calcd 253.96; found 253.96.

### Step 3: Synthesis of 1-(2-bromophenyl)-2,2,2-trifluoro-ethanone

Dess-Martin Periodinane (8.58 g, 20.2 mmol) was added portionwise to a solution of 1-(2-bromophenyl)-2,2,2-trifluoro-ethanol (4.30 g, 16.8 mmol) in DCM (100 mL). The reaction mixture was stirred at room temperature for 12 hr. The mixture was concentrated under reduced pressure. The residue was diluted MTBE (50 mL). The solid precipitate was filtered out. The filtrate was washed with saturated sodium bicarbonate solution (2×100 mL), was dried over anhydrous sodium sulfate and concentrated under reduced pressure. The residue was subjected to flash-column chromatography (SiO2, gradient elution: hexane—EtOAc) to afford 1-(2-bromophenyl)-2,2,2-trifluoro-ethanone (4.20 g, 16.6 mmol, 98.5% yield)

¹H NMR (400 MHz, CDCl3) δ 7.46-7.50 (m, 2H), 7.66-7.79 (m, 2H).

GCMS: [M−H]+ m/z: calcd 251.94; found 251.9.

### Step 4: The synthesis of 2,2,2-trifluoro-1-[2-(4,4,5,5-tetramethyl-1,3,2-dioxaborolan-2-yl)phenyl]ethanone

A mixture of 1-(2-bromophenyl)-2,2,2-trifluoro-ethanone (3.50 g, 13.8 mmol), Bis(pinacolato)diboron (3.86 g, 15.2 mmol), cesium pivalate (6.47 g, 27.7 mmol) and bis(diphenylphosphino)ferrocene]dichloropalladium(II)-DCM (72.8 mg, 89.2 μmol) in degassed dioxane (50 mL) was stirred at 85° C. for 12 hr. The mixture was cooled to room temperature and filtered. The filtrate was concentrated under reduced pressure to afford 2,2,2-trifluoro-1-[2-(4,4,5,5-tetramethyl-1,3,2-dioxaborolan-2-yl)phenyl]ethanone (3.70 g, 12.3 mmol, 81.0% yield) as a brown solid which was used in the next steps without further purification.

### Step 5: Synthesis of 2,2,2-trifluoro-[2-[8-imino-7-methyl-9-[[4-[-methyl-4-(trifluoromethyl)imidazol-2-yl]phenyl]methyl]purin-2-yl]phenyl]ethenone

2-Chloro-7-methyl-9-[[4-[1-methyl-4-(trifluoromethyl)imidazol-2-yl]phenyl]methyl]purin-8-imine (1.48 g, 3.50 mmol), Potassium carbonate (1.93 g, 14.0 mmol), 2,2,2-trifluoro-1-[2-(4,4,5,5-tetramethyl-1,3,2-dioxaborolan-2-yl)phenyl]ethanone (2.10 g, 7.00 mmol) and bis(diphenylphosphino)ferrocene]dichloropalladium(II)-DCM (36.4 mg, 44.6 μmol) were mixed in a degassed mixture of dioxane (30 mL) and water (3 mL). The reaction mixture was stirred at 90° C. for 12 hr. The reaction mixture was cooled to room temperature. The reaction mixture was diluted with EtOAc (20 mL) and washed with water (10 mL) and brine (10 mL). To the obtained organic phase Dimercaptotriazine (20 mg) was added, and the mixture was stirred for 30 min. The mixture was filtered. The filtrate was concentrated under reduced pressure. The residue was subjected to HPLC (0-5 min., 20-45% water—ACN, +0.1% vol. of formic acid, flow: 30 mL/min, column: Chromatorex 18 SB100-5T 100×19 mm, 5 μm) to afford 2,2,2-trifluoro-1-[2-[8-imino-7-methyl-9-[[4-[1-methyl-4-(trifluoromethyl)imidazol-2-yl]phenyl]methyl]purin-2-yl]phenyl]ethanone (0.20 g, 358 μmol, 5.11% yield) as a brown solid.

LCMS(ESI): [M+H]+ m/z: calcd 560.18; found 560.0.

### Step 6: The synthesis of 2,2,2-trifluoro-[2-[8-imino-7-methyl-9-[[4-[I-methyl-4-(trifluoromethyl)imidazol-2-yl]phenyl]methyl]purin-2-yl]phenyl]ethanol

Sodium Borohydride (6.76 mg, 179 μmol) was added to a stirred solution of 2,2,2-trifluoro-1-[2-[8-imino-7-methyl-9-[[4-[1-methyl-4-(trifluoromethyl)imidazol-2-yl]phenyl]methyl]purin-2-yl]phenyl]ethanone (0.10 g, 179 μmol) in MeOH (10 mL) at 0° C. The resulting mixture was stirred at this temperature for 3 hr. The reaction mixture was diluted with water (5.0 mL). The resulting mixture was stirred at room temperature for 2 hr. The mixture was concentrated under reduced pressure. The residue was subjected to HPLC (0-5 min., 30-55% water—acetonitrile, +0.1% vol. of 25% aq. NH3, flow: 30 mL/min, column: XBridge 100×19 mm, 5 μm) to afford 2,2,2-trifluoro-1-[2-[8-imino-7-methyl-9-[[4-[1-methyl-4-(trifluoromethyl)imidazol-2-yl]phenyl]methyl]purin-2-yl]phenyl]ethanol (35.0 mg, 62.3 μmol, 34.9% yield) as a brown solid.

LCMS(ESI): [M+H]+ m/z: calcd 562.2; found 562.2.

### Step 7: Chiral resolution of rel-(1R)-2,2,2-trifluoro-1-[2-[8-imino-7-methyl-9-[[4-[I-methyl-4-(trifluoromethyl)imidazol-2-yl]phenyl]methyl]purin-2-yl]phenyl]ethanol (T-148) and rel-(1S)-2,2,2-trifluoro-1-[2-[8-imino-7-methyl-9-[[4-[I-methyl-4-(trifluoromethyl)imidazol-2-yl]phenyl]methyl]purin-2-yl]phenyl]ethanol (T-129)

Racemic 2,2,2-trifluoro-1-[2-[8-imino-7-methyl-9-[[4-[1-methyl-4-(trifluoromethyl)imidazol-2-yl]phenyl]methyl]purin-2-yl]phenyl]ethanol (96.0 mg, 159 μmol) was subjected to chiral HPLC (column: Chiralpak AD-H V, 250×20 mm, 5 m; mobile phase: Hexane-IPA-MeOH, 80-10-10; flow: 12 mL/min) to afford rel-(1S)-2,2,2-trifluoro-1-[2-[8-imino-7-methyl-9-[[4-[1-methyl-4-(trifluoromethyl)imidazol-2-yl]phenyl]methyl]purin-2-yl]phenyl]ethanol (17.1 mg, 30.5 μmol, 19.2% yield) and rel-(1R)-2,2,2-trifluoro-1-[2-[8-imino-7-methyl-9-[[4-[1-methyl-4-(trifluoromethyl)imidazol-2-yl]phenyl]methyl]purin-2-yl]phenyl]ethanol (16.6 mg, 29.6 μmol, 18.6% yield) as a white solids. [1248] rel-(1S)-2,2,2-trifluoro-1-[2-[8-imino-7-methyl-9-[[4-[1-methyl-4-(trifluoromethyl)imidazol-2-yl]phenyl]methyl]purin-2-yl]phenyl]ethanol (T-129):

¹H NMR (600 MHz, DMSO-d6) δ 3.35-3.42 (m, 3H), 3.74 (s, 3H), 5.09-5.29 (m, 2H), 6.51-6.67 (m, 2H), 6.83 (s, 1H), 7.45-7.56 (m, 4H), 7.64-7.69 (m, 2H), 7.77 (d, 1H), 7.87 (d, 1H), 7.91 (s, 1H), 8.20-8.29 (m, 1H).

LCMS(ESI): [M+H]+ m/z: calcd 562.21; found 562.0.

Optical purity: 100% (column: Chiralpak AD-H, 250×4.6 mm, 5 m; mobile phase: Hexane-IPA-MeOH, 80-10-10; flow: 0.6 mL/min; RT=20.95 min)

rel-(1R)-2,2,2-trifluoro-1-[2-[8-imino-7-methyl-9-[[4-[1-methyl-4-(trifluoromethyl)imidazol-2-yl]phenyl]methyl]purin-2-yl]phenyl]ethanol (T-148):

¹H NMR (600 MHz, DMSO-d6) δ 3.35-3.42 (m, 3H), 3.74 (s, 3H), 5.09-5.29 (m, 2H), 6.51-6.67 (m, 2H), 6.83 (s, 1H), 7.45-7.56 (m, 4H), 7.64-7.69 (m, 2H), 7.77 (d, 1H), 7.87 (d, 1H), 7.91 (s, 1H), 8.20-8.29 (m, 1H).

LCMS(ESI): [M+H]+ m/z: calcd 562.21; found 562.0.

Optical purity: 100% (column: Chiralpak AD-H, 250×4.6 mm, 5 m; mobile phase: Hexane-IPA-MeOH, 80-10-10; flow: 0.6 mL/min; RT=25.65 min).

### Example T-147

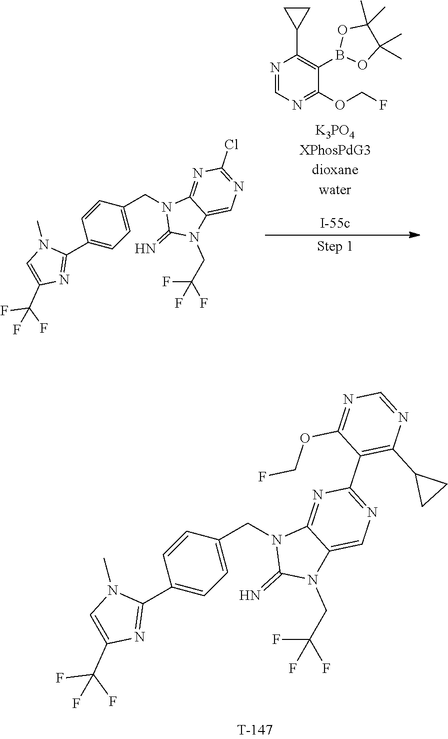 [SMILES: CC1(C)OB(c2c(OCF)ncnc2C2CC2)OC1(C)C ; Cn1cc(C(F)(F)F)nc1-c1ccc(Cn2c(=N)n(CC(F)(F)F)c3cnc(Cl)nc32)cc1 ; Cn1cc(C(F)(F)F)nc1-c1ccc(Cn2c(=N)n(CC(F)(F)F)c3cnc(-c4c(OCF)ncnc4C4CC4)nc32)cc1]

### Step 1: The synthesis of 2-[4-cyclopropyl-6-(fluoromethoxy)pyrimidin-5-yl]-9-[[4-[I-methyl-4-(trifluoromethyl)imidazol-2-yl]phenyl]methyl]-7-(2,2,2-trifluoroethyl)purin-8-imine

The synthesis of the starting 4-cyclopropyl-6-(fluoromethoxy)-5-(4,4,5,5-tetramethyl-1,3,2-dioxaborolan-2-yl)pyrimidine (I-55c) is described in Intermediate 55. [1251] 4-Cyclopropyl-6-(fluoromethoxy)-5-(4,4,5,5-tetramethyl-1,3,2-dioxaborolan-2-yl)pyrimidine (181 mg, 615 μmol), 2-chloro-9-[[4-[1-methyl-4-(trifluoromethyl)imidazol-2-yl]phenyl]methyl]-7-(2,2,2-trifluoroethyl)purin-8-imine (108 mg, 220 μmol), potassium phosphate tribasic anhydrous (140 mg, 659 μmol) and XPhosPdG3 (12.9 mg, 15.3 μmol) were mixed in a degassed mixture of dioxane (6.0 mL) and water (600 μL) under argon atmosphere.

The reaction mixture was stirred at 75° C. for 12 hr. The reaction mixture was cooled to room temperature and subjected to HPLC (2-10 min., 35-50% ACN, +0.1% vol. of 25% aq. NH3, flow: 30 mL/min, column: YMC-ACTUS TRIART 100×20 mm, 5 μm) to afford 2-[4-cyclopropyl-6-(fluoromethoxy)pyrimidin-5-yl]-9-[[4-[1-methyl-4-(trifluoromethyl)imidazol-2-yl]phenyl]methyl]-7-(2,2,2-trifluoroethyl)purin-8-imine (18.0 mg, 29.0 μmol, 13.2% yield) as a yellow solid.

¹H NMR (600 MHz, DMSO-d6) δ 0.87-0.93 (m, 2H), 1.04-1.08 (m, 2H), 1.75-1.81 (m, 1H), 3.75 (s, 3H), 4.80-5.04 (m, 2H), 5.10-5.26 (m, 2H), 6.05 (d, 2H, CH2F), 7.11-7.17 (m, 1H), 7.51 (d, 2H), 7.64-7.69 (m, 2H), 7.92 (s, 1H), 8.34-8.41 (m, 1H), 8.73 (s, 1H).

LCMS(ESI): [M+H]+ m/z: calcd 622.22; found 622.4.

### Example T-144

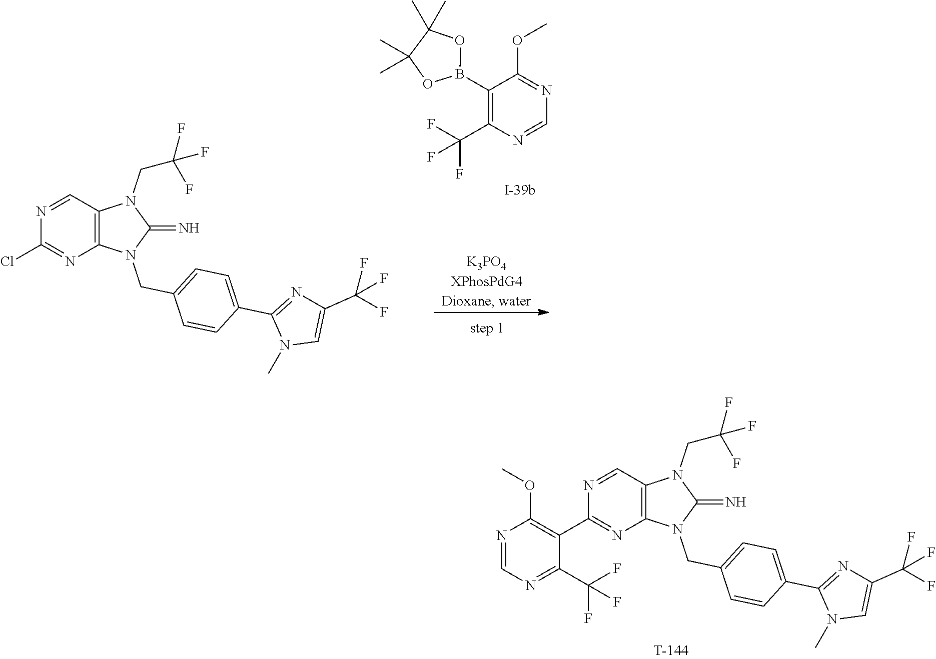

The synthesis of 4-methoxy-5-(4,4,5,5-tetramethyl-1,3,2-dioxaborolan-2-yl)-6-(trifluoromethyl)pyrimidine is described by Intermediate 39.

### Step 1: The synthesis of 2-[4-methoxy-6-(trifluoromethyl)pyrimidin-5-yl]-9-[[4-[I-methyl-4-(trifluoromethyl)imidazol-2-yl]phenyl]methyl]-7-(2,2,2-trifluoroethyl)purin-8-imine

2-Chloro-9-[[4-[1-methyl-4-(trifluoromethyl)imidazol-2-yl]phenyl]methyl]-7-(2,2,2-trifluoroethyl)purin-8-imine (80.0 mg, 163 μmol), 4-methoxy-5-(4,4,5,5-tetramethyl-1,3,2-dioxaborolan-2-yl)-6-(trifluoromethyl)pyrimidine (149 mg, 490 μmol), potassium phosphate tribasic anhydrous (104 mg, 490 μmol) and RuPhos Pd G4 (13.9 mg, 16.3 μmol) were mixed in a degassed mixture of dioxane (3 mL) and water (0.3 mL). The mixture was stirred at 90° C. for 12 hr. under argon atmosphere. The reaction mixture was cooled to room temperature. The reaction mixture was diluted with EtOAc (20 mL) and washed with water (10 mL) and brine (10 mL). To the obtained organic phase Dimercaptotriazine (20 mg) was added, and the mixture was stirred for 30 min. The mixture was filtered. The filtrate was concentrated under reduced pressure. The residue was subjected to HPLC (2-10 min, 27-50% water+FA (0.1% vol.)—ACN+FA (0.1% vol.); flow: 30 mL/min, column: Waters SunFire C18, 100×19 mm, 5 μm) to afford 2-[4-methoxy-6-(trifluoromethyl)pyrimidin-5-yl]-9-[[4-[1-methyl-4-(trifluoromethyl)imidazol-2-yl]phenyl]methyl]-7-(2,2,2-trifluoroethyl)purin-8-imine (7.00 mg, 11.1 μmol, 6.79% yield) as a light-yellow solid.

¹H NMR (600 MHz, DMSO-d6) δ 3.74 (s, 3H), 3.97 (s, 3H), 4.80-5.04 (m, 2H), 5.07-5.24 (m, 2H), 7.17-7.24 (m, 1H), 7.46 (d, 2H), 7.64-7.68 (m, 2H), 7.92 (s, 1H), 8.31-8.38 (m, 1H), 9.08 (s, 1H).

LCMS(ESI): [M+H]+ m/z: calcd 632.18; found 632.2.

### Example T-102

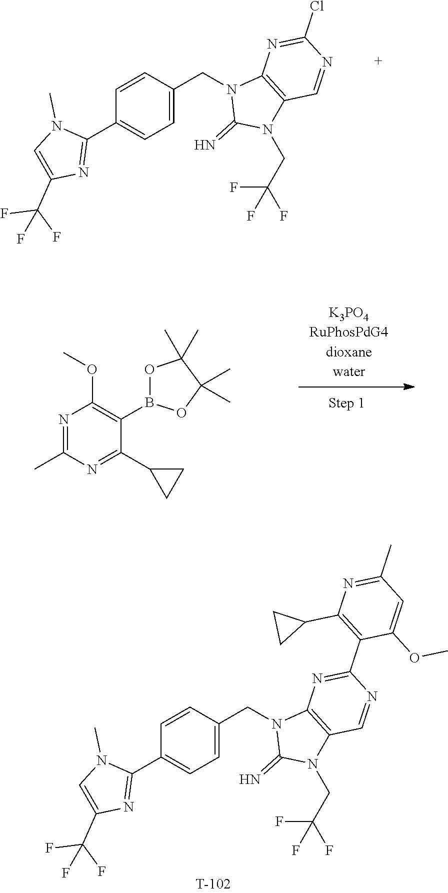

### Step 1: The synthesis of 2-(4-cyclopropyl-6-methoxy-2-methyl-pyrimidin-5-yl)-9-[[4-[I-methyl-4-(trifluoromethyl)imidazol-2-yl]phenyl]methyl]-7-(2,2,2-trifluoroethyl)purin-8-imine

2-Chloro-9-[[4-[1-methyl-4-(trifluoromethyl)imidazol-2-yl]phenyl]methyl]-7-(2,2,2-trifluoroethyl)purin-8-imine (38.0 mg, 77.6 μmol), 4-cyclopropyl-6-methoxy-2-methyl-5-(4,4,5,5-tetramethyl-1,3,2-dioxaborolan-2-yl)pyrimidine (49.5 mg, 171 μmol), RuPhosPdG4 (6.59 mg, 7.76 μmol) and potassium phosphate tribasic (32.9 mg, 155 μmol) were mixed in a degassed mixture of dioxane (7.0 mL) and water (500 μL) under argon atmosphere at room temperature. The reaction mixture was stirred at 100° C. for 12 hr. The reaction mixture was cooled to room temperature and filtered. The filtrate was concentrated under reduced pressure. The residue was subjected to HPLC (0-5 min., 35-60% water—ACN, +0.1% vol. of 25% aq. NH3, flow: 30 mL/min, column: XBridge C18 100×19 mm, 5 μm) to afford 2-(4-cyclopropyl-6-methoxy-2-methyl-pyrimidin-5-yl)-9-[[4-[1-methyl-4-(trifluoromethyl)imidazol-2-yl]phenyl]methyl]-7-(2,2,2-trifluoroethyl)purin-8-imine (6.00 mg, 9.72 μmol, 12.5% yield) as a brown solid.

¹H NMR (600 MHz, DMSO-d6) δ 0.75-0.79 (m, 2H), 0.94-0.99 (m, 2H), 1.62-1.68 (m, 1H), 2.45 (s, 3H), 3.73 (s, 3H), 3.79 (s, 3H), 4.77-5.01 (m, 2H), 5.06-5.23 (m, 2H), 7.03-7.09 (m, 1H), 7.48 (d, 2H), 7.62-7.68 (m, 2H), 7.90 (s, 1H), 8.28-8.35 (m, 1H).

LCMS(ESI): [M+H]+ m/z: calcd 618.25; found 618.0.

### Example T-107

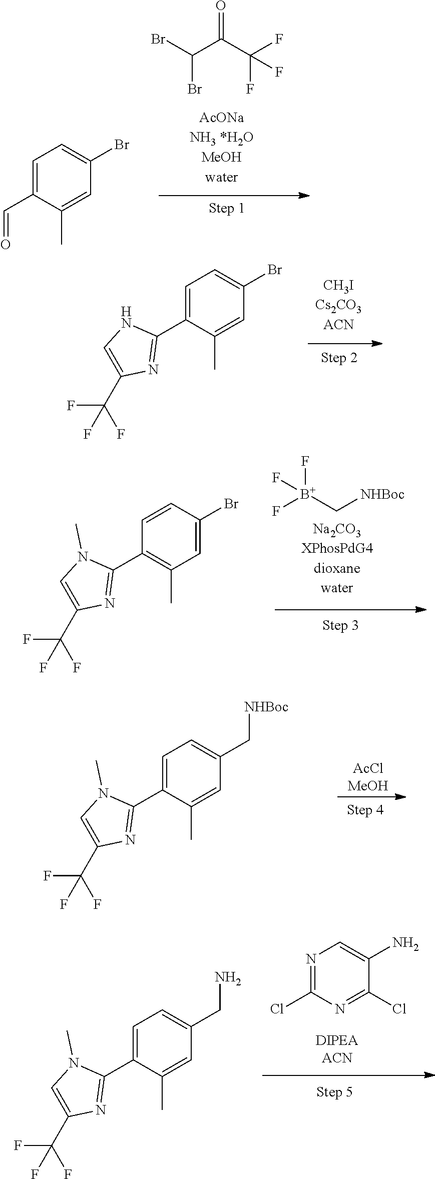

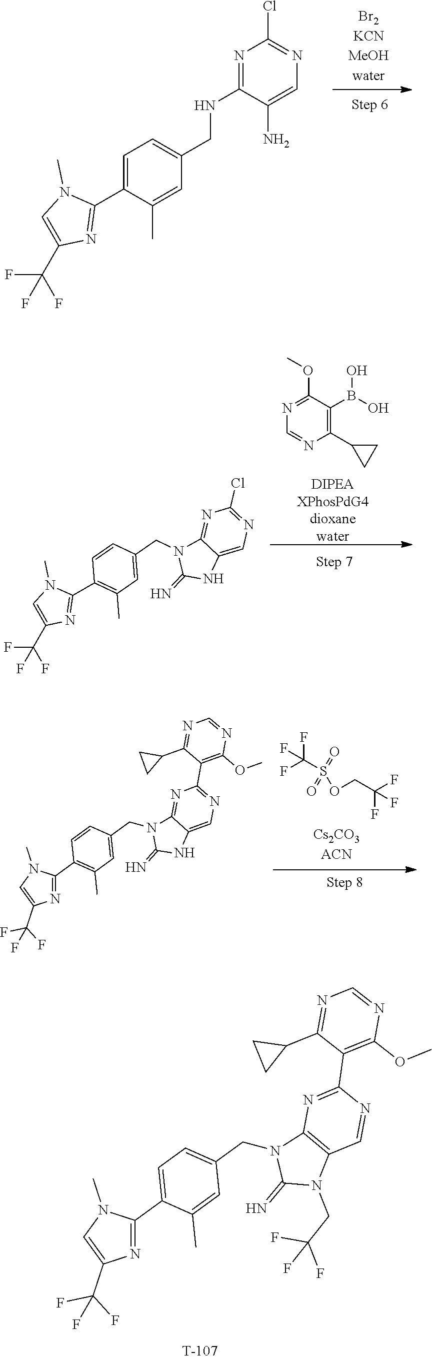

### Step 1: The synthesis of 2-(4-bromo-2-methyl-phenyl)-4-(trifluoromethyl)-1H-imidazole

3,3-dibromo-1,1,1-trifluoro-propan-2-one (7.46 g, 27.6 mmol) was added to a solution of sodium acetate (4.74 g, 57.8 mmol) in water (70 mL). The resulting mixture was stirred at 95° C. for 1 hr. The mixture was cooled to room temperature and poured into a solution of 4-bromo-2-methyl-benzaldehyde (5.00 g, 25.1 mmol) and aqueous NH4OH (25 mL, 25% wt.) in MeOH (250 mL). The reaction mixture was stirred at room temperature for 16 hr. The reaction mixture was concentrated under reduced pressure to ˜30 mL. The residue was diluted with water (100 mL) and extracted with EtOAc (3×100 mL). The combined organic layers were washed with brine (50 mL), dried over anhydrous sodium sulfate and filtered. The filtrate was concentrated under reduced pressure. The residue was recrystalized from MTBE to afford 2-(4-bromo-2-methyl-phenyl)-4-(trifluoromethyl)-1H-imidazole (2.74 g, 8.98 mmol, 35.8% yield) as a white solid which was used in the next steps without further purification.

LCMS(ESI): [M+H]+ m/z: calcd 304.99; found 304.8.

### Step 2: The synthesis of 2-(4-bromo-2-methyl-phenyl)-1-methyl-4-(trifluoromethyl)imidazole

Cesium carbonate (4.78 g, 14.7 mmol) and methyl iodide (1.15 g, 8.08 mmol, 503 μL) were added to a stirred solution of 2-(4-bromo-2-methyl-phenyl)-4-(trifluoromethyl)-1H-imidazole (2.24 g, 7.34 mmol) in ACN (70 mL). The reaction mixture was stirred at room temperature for 16 hr. The reaction mixture was diluted with MTBE (50 mL). The resulting mixture was stirred at room temperature for 5 min, then solids were filtered out. The filtrate was concentrated under reduced pressure. The residue was subjected to column chromatography (SiO2, gradient MTBE in Hexane, from 0 to 16.5% MTBE) to afford 2-(4-bromo-2-methyl-phenyl)-1-methyl-4-(trifluoromethyl)imidazole (1.20 g, 3.76 mmol, 51.2% yield) as a light-yellow oil.

¹H NMR (400 MHz, CDCl3) δ 2.21 (s, 3H), 3.52 (s, 3H), 7.17 (d, 1H), 7.33 (s, 1H), 7.42 (d, 1H), 7.48 (s, 1H).

LCMS(ESI): [M+H]+ m/z: calcd 319.03; found 319.0.

### Step 3: The synthesis of tert-butyl N-[[3-methyl-4-[-methyl-4-(trifluoromethyl)imidazol-2-yl]phenyl]methyl]carbamate

Sodium carbonate (897 mg, 8.46 mmol) and potassium (tert-butoxycarbonylamino)methyl-trifluoro-boranuide (1.20 g, 5.08 mmol) were added to a stirred solution of 2-(4-bromo-2-methyl-phenyl)-1-methyl-4-(trifluoromethyl)imidazole (900 mg, 2.82 mmol) in a degassed mixture of dioxane (40 mL) and water (8.0 mL). The resulting mixture was degassed. XPhosPdG4 (72.8 mg, 84.6 μmol) was added to the mixture. The reaction mixture was stirred at 95° C. for 16 hr under argon atmosphere. The reaction mixture was cooled to room temperature and filtered through a pad of silica gel. The filtrate was concentrated under reduced pressure. The residue was dissolved in MTBE (75 mL), then solids were filtered out. The filtrate was concentrated under reduced pressure to afford tert-butyl N-[[3-methyl-4-[1-methyl-4-(trifluoromethyl)imidazol-2-yl]phenyl]methyl]carbamate (1.26 g, crude) as a light-yellow solid which was used in the next steps without further purification.

LCMS(ESI): [M+H]+ m/z: calcd 370.21; found 370.2.

### Step 4: The synthesis of [3-methyl-4-[1-methyl-4-(trifluoromethyl)imidazol-2-yl]phenyl]methanamine

Acetyl chloride (1.34 g, 17.1 mmol, 1.03 mL) was added dropwise to a stirred MeOH (75 mL) at 5° C. Tert-butyl N-[[3-methyl-4-[1-methyl-4-(trifluoromethyl)imidazol-2-yl]phenyl]methyl]carbamate (1.26 g, crude) was added to the resulting solution. The reaction mixture was stirred at room temperature for 16 hr. The reaction mixture was concentrated under reduced pressure. The residue was diluted with MTBE (20 mL). The solid precipitate formed was filtered off, washed with MTBE (3×20 mL) and dried under reduced pressure to afford [3-methyl-4-[1-methyl-4-(trifluoromethyl)imidazol-2-yl]phenyl]methanamine (1.15 g, crude, HCl) as a light-yellow solid which was used in the next steps without further purification.

LCMS(ESI): [M+H]+ m/z: calcd 270.15; found 270.2.

### Step 5: The synthesis of 2-chloro-N4-[[3-methyl-4-[1-methyl-4-(trifluoromethyl)imidazol-2-yl]phenyl]methyl]pyrimidine-4,5-diamine

DIPEA (1.14 g, 8.83 mmol, 1.54 mL) and 2,4-dichloropyrimidin-5-amine (483 mg, 2.94 mmol) were added to a stirred solution of [3-methyl-4-[1-methyl-4-(trifluoromethyl)imidazol-2-yl]phenyl]methanamine (900 mg, HCl) in ACN (40 mL) under argon atmosphere. The reaction mixture was stirred at 75° C. for 40 hr. The reaction mixture was cooled to room temperature and concentrated under reduced pressure. The residue was diluted with water (100 mL) and extracted with EtOAc (3×100 mL). The combined organic layers were washed with brine (100 mL), dried over anhydrous sodium sulfate and filtered. The filtrate was concentrated under reduced pressure to afford 2-chloro-N4-[[3-methyl-4-[1-methyl-4-(trifluoromethyl)imidazol-2-yl]phenyl]methyl]pyrimidine-4,5-diamine (1.60 g, crude) as a red gum which was used in the next steps without further purification.

LCMS(ESI): [M+H]+ m/z: calcd 397.14; found 397.0.

### Step 6: The synthesis of 2-chloro-9-[[3-methyl-4-[-methyl-4-(trifluoromethyl)imidazol-2-yl]phenyl]methyl]-7H-purin-8-imine

A solution of potassium cyanide (1.18 g, 18.2 mmol) in water (45 mL) was added to a precooled to 0° C. stirred solution of Br2 (2.90 g, 18.2 mmol) in water (15 mL). The resulting mixture was stirred at 0° C. for 15 min. A solution of 2-chloro-N4-[[3-methyl-4-[1-methyl-4-(trifluoromethyl)imidazol-2-yl]phenyl]methyl]pyrimidine-4,5-diamine (1.60 g, crude) in MeOH (120 mL) was added to the mixture at 0° C. The reaction mixture was stirred at 50° C. for 16 hr. The reaction mixture was cooled to room temperature and concentrated under reduced pressure. The residue was diluted with water (30 mL) and extracted with EtOAc (3×75 mL). The combined organic layers were washed with brine (25 mL), dried over anhydrous sodium sulfate and filtered. The filtrate was concentrated under reduced pressure. The residue was subjected to column chromatography (SiO2, gradient MTBE—MeOH) to afford 2-chloro-9-[[3-methyl-4-[1-methyl-4-(trifluoromethyl)imidazol-2-yl]phenyl]methyl]-7H-purin-8-imine (330 mg, 782 μmol, 26.6% yield from 2,4-dichloropyrimidin-5-amine) as a red solid.

¹H NMR (500 MHz, DMSO-d6) δ 2.11 (s, 3H), 3.45 (s, 3H), 5.30 (s, 1H), 7.05 (d, 1H), 7.23 (s, 1H), 7.34 (d, 1H), 7.48 (s, 2H), 7.90 (s, 1H), 8.30 (s, 1H).

LCMS(ESI): [M+H]+ m/z: calcd 422.13; found 422.0.

### Step 7: The synthesis of 2-(4-cyclopropyl-6-methoxy-pyrimidin-5-yl)-9-[[3-methyl-4-[I-methyl-4-(trifluoromethyl)imidazol-2-yl]phenyl]methyl]-7H-purin-8-imine

(4-Cyclopropyl-6-methoxy-pyrimidin-5-yl)boronic acid (40.5 mg, 209 μmol) and sodium carbonate (60.3 mg, 569 μmol) were added to a stirred solution of 2-chloro-9-[[3-methyl-4-[1-methyl-4-(trifluoromethyl)imidazol-2-yl]phenyl]methyl]-7H-purin-8-imine (80 mg, 190 μmol) in a degassed mixture of dioxane (6.0 mL) and water (1.5 mL) under argon atmosphere. XPhos Pd G4 (8.16 mg, 9.48 μmol) was added to the mixture. The reaction mixture was stirred at 90° C. for 16 hr under argon atmosphere. The reaction mixture was cooled to room temperature and filtered through a pad of silica gel. The filtrate was concentrated under reduced pressure to afford 2-(4-cyclopropyl-6-methoxy-pyrimidin-5-yl)-9-[[3-methyl-4-[1-methyl-4-(trifluoromethyl)imidazol-2-yl]phenyl]methyl]-7H-purin-8-imine (155 mg, crude) as a yellow solid which was used in the next step without further purification.

LCMS(ESI): [M+H]+ m/z: calcd 536.22; found 536.0.

### Step 8: The synthesis of 2-(4-cyclopropyl-6-methoxy-pyrimidin-5-yl)-9-[[3-methyl-4-[I-methyl-4-(trifluoromethyl)imidazol-2-yl]phenyl]methyl]-7-(2,2,2-trifluoroethyl)purin-8-imine

Cesium carbonate (189 mg, 579 μmol) and 2,2,2-trifluoroethyl trifluoromethanesulfonate (70.5 mg, 304 μmol) were added to a stirred solution of 2-(4-cyclopropyl-6-methoxy-pyrimidin-5-yl)-9-[[3-methyl-4-[1-methyl-4-(trifluoromethyl)imidazol-2-yl]phenyl]methyl]-7H-purin-8-imine (155 mg, 289 μmol) in ACN (25.0 mL) at room temperature. The reaction mixture was stirred at 70° C. for 16 hr. The reaction mixture was cooled to room temperature and concentrated under reduced pressure. The residue was dissolved in EtOAc (40 mL), then solids were filtered out. The filtrate was concentrated under reduced pressure. The residue was subjected to HPLC (0-1-6 min, 30-30-60% water+FA (0.2% vol.)—ACN+FA (0.2% vol.); flow: 30 mL/min, column: Chromatorex 18 SMB 100-5T, 100×19 mm, 5 μm), then repurified by SFC (eluent: CO2-MeOH, 5-50, 50.0 mL/min (Add-on: 5.0 ml/min), column: Chromatorex PEI (19×100 mm, 5 m)) to afford 2-(4-cyclopropyl-6-methoxy-pyrimidin-5-yl)-9-[[3-methyl-4-[1-methyl-4-(trifluoromethyl)imidazol-2-yl]phenyl]methyl]-7-(2,2,2-trifluoroethyl)purin-8-imine (5.20 mg, 8.42 μmol, 2.91% yield) as an off-white solid.

¹H NMR (600 MHz, DMSO-d6) δ 0.80-0.84 (m, 2H), 0.98-1.01 (m, 2H), 1.65-1.69 (m, 1H), 2.09 (s, 3H), 3.45 (s, 3H), 3.81 (s, 3H), 4.77-5.02 (m, 2H), 5.06-5.18 (m, 2H), 7.00-7.09 (m, 1H), 7.23-7.28 (m, 1H), 7.30-7.37 (m, 2H), 7.90 (s, 1H), 8.30-8.37 (m, 1H), 8.62 (s, 1H).

LCMS(ESI): [M+H]+ m/z: calcd 618.22; found 618.2.

### Example T-137

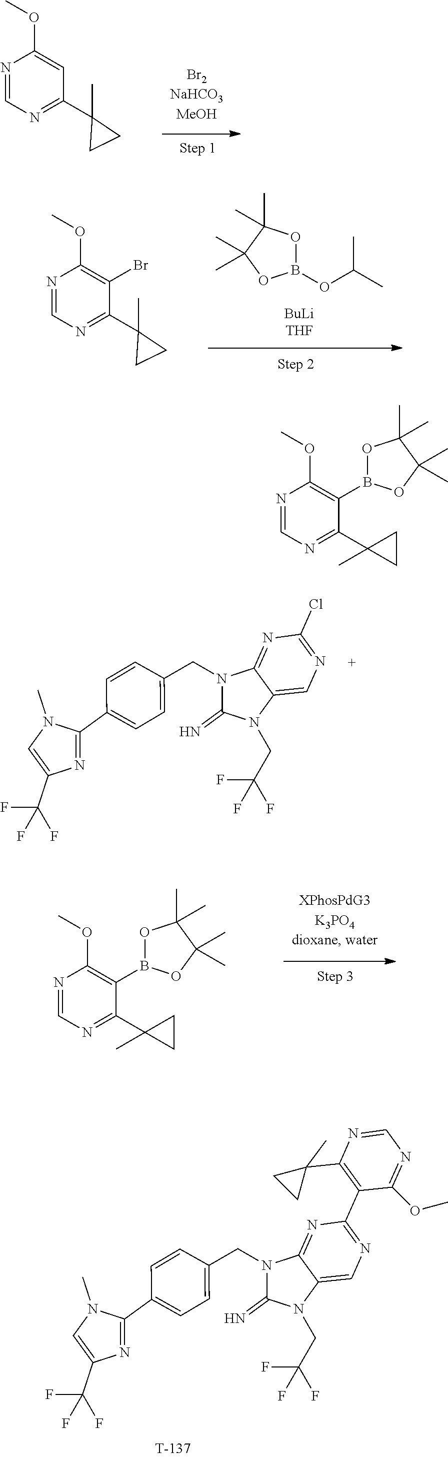

### Step 1: The synthesis of 5-bromo-4-methoxy-6-(1-methylcyclopropyl)pyrimidine

To a stirred solution of 4-methoxy-6-(1-methylcyclopropyl)pyrimidine (2.70 g, 16.4 mmol) in MeOH (50 mL) Sodium hydrogen carbonate (2.76 g, 32.9 mmol, 1.28 mL) and bromine (5.26 g, 32.9 mmol) were added sequentially at 0° C. The reaction mixture was stirred at room temperature for 72 hr. The reaction mixture was concentrated under reduced pressure. The residue was diluted with EtOAc (30 mL) and washed with water (30 mL). The organic layer was separated, washed with an aqueous solution of Na2S2O3 (20 mL, 5% wt.), water (10 mL) and brine (15 mL), dried over anhydrous sodium sulfate and concentrated under reduced pressure. The residue was subjected to flash-column chromatography (SiO2, gradient hexane—MTBE) to afford 5-bromo-4-methoxy-6-(1-methylcyclopropyl)pyrimidine (2.40 g, 9.87 mmol, 60.0% yield) as a light-yellow oil.

¹H NMR (500 MHz, CDCl3) δ 0.84-0.88 (m, 2H), 0.97-1.01 (m, 2H), 1.43 (s, 3H), 4.05 (s, 3H), 8.57 (s, 1H).

LCMS(ESI): [M+H]+ m/z: calcd 243.01, 245.01; found 243.0, 245.0.

### Step 2: The synthesis of 4-methoxy-6-(1-methylcyclopropyl)-5-(4,4,5,5-tetramethyl-1,3,2-dioxaborolan-2-yl)pyrimidine

n-Butyllithium (7.40 mmol, 3.36 mL, 2.2M in hexane) was added dropwise to a precooled to −78° C. solution of 5-bromo-4-methoxy-6-(1-methylcyclopropyl)pyrimidine (1.20 g, 4.94 mmol) and 2-isopropoxy-4,4,5,5-tetramethyl-1,3,2-dioxaborolane (1.38 g, 7.40 mmol) in THF (50 mL) under argon atmosphere. The reaction mixture was allowed to warm to room temperature and stirred for 3 hr. The reaction mixture was quenched by dropwise addition of water (20 mL) and extracted with EtOAc (50 mL). The organic layer was washed with brine (10 mL), dried over anhydrous sodium sulfate and concentrated under reduced pressure. The residue was subjected to flash-column chromatography (SiO2, gradient hexane—MTBE) to afford 4-methoxy-6-(1-methylcyclopropyl)-5-(4,4,5,5-tetramethyl-1,3,2-dioxaborolan-2-yl)pyrimidine (740 mg, 2.55 mmol, 51.7% yield) as a light-yellow oil.

LCMS(ESI): [M+H]+ m/z: calcd 291.22; found 291.2.

### Step 3: The synthesis of 2-[4-methoxy-6-(1-methylcyclopropyl)pyrimidin-5-yl]-9-[[4-[1-methyl-4-(trifluoromethyl)imidazol-2-yl]phenyl]methyl]-7-(2,2,2-trifluoroethyl)purin-8-imine

2-chloro-9-[[4-[1-methyl-4-(trifluoromethyl)imidazol-2-yl]phenyl]methyl]-7-(2,2,2-trifluoroethyl)purin-8-imine (40.0 mg, 81.7 μmol), 4-methoxy-6-(1-methylcyclopropyl)-5-(4,4,5,5-tetramethyl-1,3,2-dioxaborolan-2-yl)pyrimidine (35.5 mg, 123 μmol), potassium phosphate tribasic anhydrous (52.0 mg, 245 μmol) and XPhosPdG3 (3.46 mg, 4.09 μmol) were mixed in degassed mixture of dioxane (4.5 mL) and water (500 μL). The reaction mixture was stirred at 90° C. for 12 hr. under argon atmosphere. The reaction mixture was cooled to room temperature, diluted with water (5.0 mL) and extracted with EtOAc (8.0 mL). The organic layer was washed with brine (5.0 mL) and dried over anhydrous sodium sulfate. To the resulting mixture SiliaMetS® Dimercaptotriazine (50.0 mg) was added and the mixture was stirred for 30 min. The mixture was filtered. The filtrate was concentrated under reduced pressure. The residue was subjected to HPLC (0.5-6.5 min, 14-29% water+FA (0.1% vol.)—ACN+FA (0.1% vol.); flow: 30 mL/min, column: SunFire C18, 100×19 mm, 5 μm) to afford 2-[4-methoxy-6-(1-methylcyclopropyl)pyrimidin-5-yl]-9-[[4-[1-methyl-4-(trifluoromethyl)imidazol-2-yl]phenyl]methyl]-7-(2,2,2-trifluoroethyl)purin-8-imine (9.00 mg, 14.6 μmol, 17.9% yield) as a yellow solid.

¹H NMR (600 MHz, DMSO-d6) δ 0.32 (s, 2H), 0.65 (s, 2H), 1.00 (s, 3H), 3.71 (s, 3H), 3.81 (s, 3H), 4.79-5.05 (m, 2H), 5.08-5.26 (m, 2H), 7.05-7.14 (m, 1H), 7.38-7.46 (m, 2H), 7.60-7.69 (m, 2H), 7.90 (s, 1H), 8.29-8.38 (m, 1H), 8.70 (s, 1H).

LCMS(ESI): [M+H]+ m/z: calcd 618.25; found 618.2.

### Example T-122

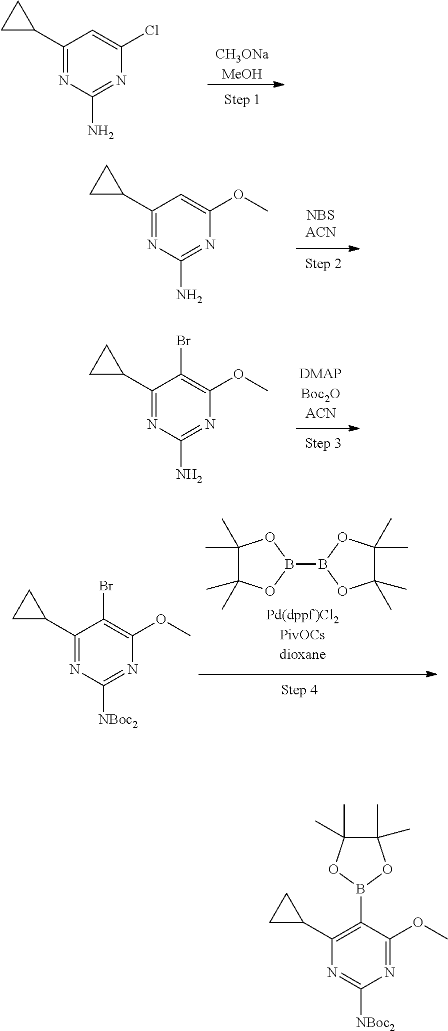

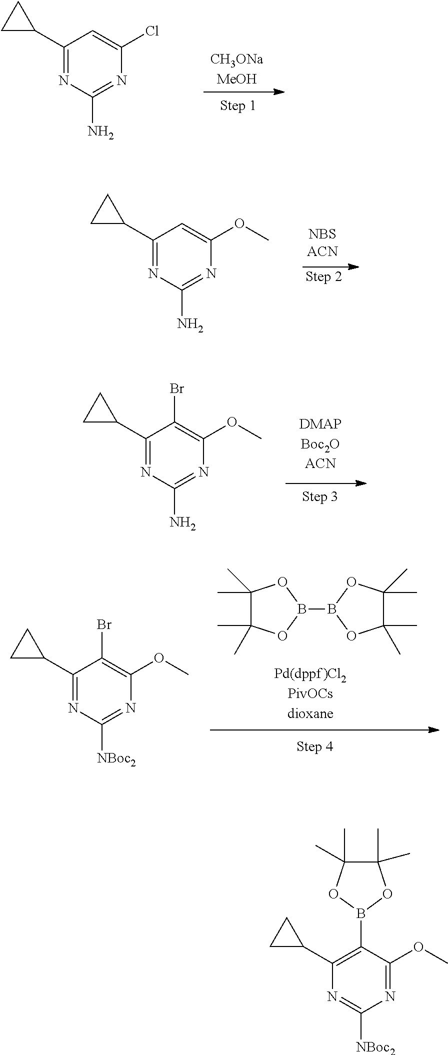

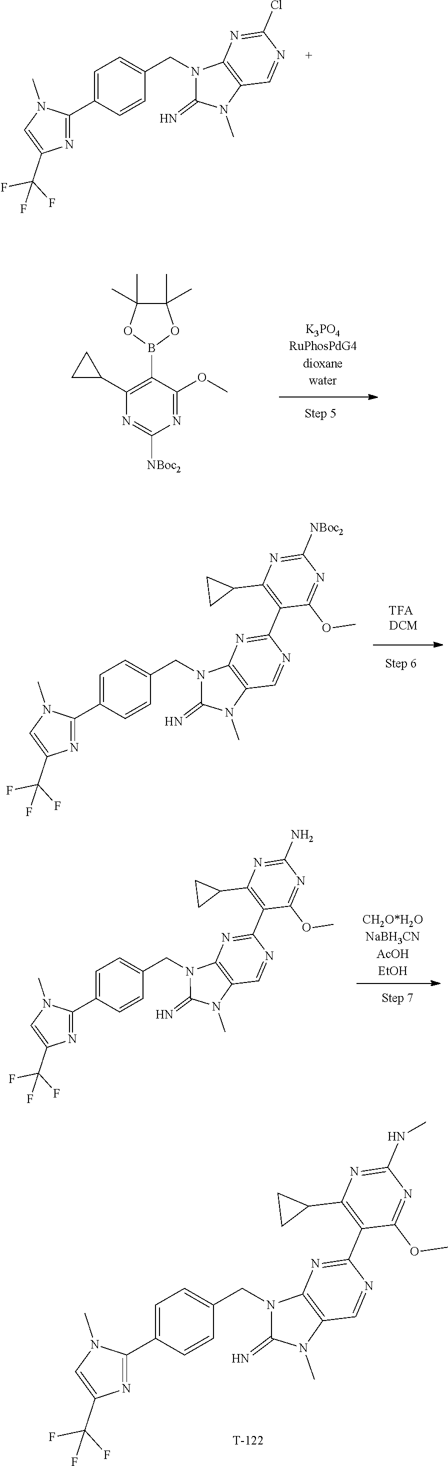

### Step 1: The synthesis of 4-cyclopropyl-6-methoxy-pyrimidin-2-amine

4-chloro-6-cyclopropyl-pyrimidin-2-amine (5.00 g, 29.5 mmol) was added to a solution of sodium methoxide (3.18 g, 59.0 mmol) in MeOH (100 mL). The reaction mixture was stirred at 65° C. for 15 hr. The reaction mixture was cooled to room temperature and concentrated under reduced pressure. The residue was diluted with water (50 mL). The solids were filtered off and dried on air to afford 4-cyclopropyl-6-methoxy-pyrimidin-2-amine (4.00 g, 24.2 mmol, 82.1% yield) as a white solid which was used in the next steps without further purification.

¹H NMR (500 MHz, DMSO-d6) δ 0.75-0.87 (m, 4H), 1.69-1.75 (m, 1H), 3.71 (s, 3H), 5.80 (s, 1H).

### Step 2: The synthesis of 5-bromo-4-cyclopropyl-6-methoxy-pyrimidin-2-amine

N-Bromosuccinimide (5.09 g, 28.6 mmol) was added to a solution of 4-cyclopropyl-6-methoxy-pyrimidin-2-amine (4.50 g, 27.2 mmol) in ACN (20 mL). The reaction mixture was stirred at room temperature for 15 hr. The white participate was formed filtered off to afford 5-bromo-4-cyclopropyl-6-methoxy-pyrimidin-2-amine (5.00 g, 20.5 mmol, 75.2% yield) as a white solid which was used in the next steps without further purification.

¹H NMR (500 MHz, CDCl3) δ 0.92-0.99 (m, 2H), 1.03-1.08 (m, 2H), 2.37-2.43 (m, 1H), 3.92 (s, 3H), 4.76 (br., 2H).

LCMS(ESI): [M+H]+ m/z: calcd 244.01, 246.01; found 244.0, 246.0.

### Step 3: The synthesis of tert-butyl N-(5-bromo-4-cyclopropyl-6-methoxy-pyrimidin-2-yl)-N-tert-butoxycarbonyl-carbamate

DMAP (751 mg, 6.15 mmol) and di-tert-butyl dicarbonate (805 mg, 3.69 mmol, 846 μL) were added to a solution of 5-bromo-4-cyclopropyl-6-methoxy-pyrimidin-2-amine (3.00 g, 12.3 mmol) in ACN (16 mL). The reaction mixture was stirred at 80° C. for 15 hr. The reaction mixture was cooled to room temperature and concentrated under reduced pressure. The residue was subjected to flash column chromatography SiO2, gradient hexane—MTBE) to afford tert-butyl N-(5-bromo-4-cyclopropyl-6-methoxy-pyrimidin-2-yl)-N-tert-butoxycarbonyl-carbamate (3.90 g, 8.78 mmol, 71.4% yield) as a yellow solid.

¹H NMR (400 MHz, CDCl3) δ 1.01-1.09 (m, 2H), 1.09-1.14 (m, 2H), 1.43 (s, 18H), 2.45-2.52 (m, 1H), 3.99 (s, 3H).

LCMS(ESI): [M+H]+ m/z: calcd 344.06; found 344.0.

### Step 4: The synthesis of tert-butyl N-tert-butoxycarbonyl-N-[4-cyclopropyl-6-methoxy-5-(4,4,5,5-tetramethyl-1,3,2-dioxaborolan-2-yl)pyrimidin-2-yl]carbamate

Tert-butyl N-(5-bromo-4-cyclopropyl-6-methoxy-pyrimidin-2-yl)-N-tert-butoxycarbonyl-carbamate (1.60 g, 3.60 mmol), 4,4,5,5-tetramethyl-2-(4,4,5,5-tetramethyl-1,3,2-dioxaborolan-2-yl)-1,3,2-dioxaborolane (1.37 g, 5.40 mmol), bis(diphenylphosphino)ferrocene]dichloropalladium(II)-DCM (294 mg, 360 μmol) and cesium pivalate (2.11 g, 9.00 mmol) were mixed in degassed dioxane (30 mL) under argon atmosphere at room temperature. The reaction mixture was stirred at 90° C. for 15 hr. The reaction mixture was cooled to room temperature and concentrated under reduced pressure. The residue was diluted with water (30 mL) and extracted with EtOAc (2×40 mL). The combined organic layers were dried over anhydrous sodium sulfate and concentrated under reduced pressure to afford tert-butyl N-tert-butoxycarbonyl-N-[4-cyclopropyl-6-methoxy-5-(4,4,5,5-tetramethyl-1,3,2-dioxaborolan-2-yl)pyrimidin-2-yl]carbamate (3.10 g, crude) as a brown solid which was used in the next steps without further purification.

LCMS(ESI): [M+H]+ m/z: calcd 492.29; found 492.2.

### Step 5: The synthesis of tert-butyl N-tert-butoxycarbonyl-N-[4-cyclopropyl-5-[8-imino-7-methyl-9-[[4-[1-methyl-4-(trifluoromethyl)imidazol-2-yl]phenyl]methyl]purin-2-yl]-6-methoxy-pyrimidin-2-yl]carbamate

2-chloro-7-methyl-9-[[4-[1-methyl-4-(trifluoromethyl)imidazol-2-yl]phenyl]methyl]purin-8-imine (300 mg, 711 μmol), tert-butyl N-tert-butoxycarbonyl-N-[4-cyclopropyl-6-methoxy-5-(4,4,5,5-tetramethyl-1,3,2-dioxaborolan-2-yl)pyrimidin-2-yl]carbamate (1.08 g, crude), RuPhosPdG4 (45.4 mg, 53.3 μmol) and potassium phosphate tribasic anhydrous (453 mg, 2.13 mmol) were mixed in a degassed mixture of dioxane (15 mL) and water (3.0 mL) under argon atmosphere at room temperature. The reaction mixture was stirred at 90° C. for 15 hr. The reaction mixture was cooled to room temperature and concentrated under reduced pressure. The residue was diluted with water (10 mL) and extracted with DCM (2×50 mL). The combined organic layers were dried over anhydrous sodium sulfate and concentrated under reduced pressure to afford tert-butyl N-tert-butoxycarbonyl-N-[4-cyclopropyl-5-[8-imino-7-methyl-9-[[4-[1-methyl-4-(trifluoromethyl)imidazol-2-yl]phenyl]methyl]purin-2-yl]-6-methoxy-pyrimidin-2-yl]carbamate (700 mg, crude) as a brown solid which was used in the next steps without further purification.

LCMS(ESI): [M+H]+ m/z: calcd 751.33; found 751.2.

### Step 6: The synthesis of 4-cyclopropyl-5-[8-imino-7-methyl-9-[[4-[I-methyl-4-(trifluoromethyl)imidazol-2-yl]phenyl]methyl]purin-2-yl]-6-methoxy-pyrimidin-2-amine

TFA (2.13 g, 18.7 mmol, 1.44 mL) was added to a solution of tert-butyl N-tert-butoxycarbonyl-N-[4-cyclopropyl-5-[8-imino-7-methyl-9-[[4-[1-methyl-4-(trifluoromethyl)imidazol-2-yl]phenyl]methyl]purin-2-yl]-6-methoxy-pyrimidin-2-yl]carbamate (700 mg, crude) in DCM (10 mL). The reaction mixture was stirred at room temperature for 15 hr. The reaction mixture was concentrated under reduced pressure. The residue was subjected to HPLC (2-10 min, 0-45% ACN+FA, flow 30 ml/min; column SunFire C18 100×19 mm 5 m) to afford 4-cyclopropyl-5-[8-imino-7-methyl-9-[[4-[1-methyl-4-(trifluoromethyl)imidazol-2-yl]phenyl]methyl]purin-2-yl]-6-methoxy-pyrimidin-2-amine (199 mg, 362 μmol, 50.9% yield from 2-chloro-7-methyl-9-[[4-[1-methyl-4-(trifluoromethyl)imidazol-2-yl]phenyl]methyl]purin-8-imine) as an off-white solid.

¹H NMR (DMSO-d6, 600 MHz) δ 0.61-0.66 (m, 2H), 0.86-0.90 (m, 2H), 1.57-1.63 (m, 1H), 3.31-3.37 (m, 3H), 3.69 (s, 3H), 3.72 (s, 3H), 5.04-5.20 (m, 2H), 6.37-6.53 (m, 3H), 7.48 (d, 2H), 7.64 (d, 2H), 7.90 (s, 1H), 8.11-8.20 (m, 1H).

LCMS(ESI): [M+H]+ m/z: calcd 551.25; found 551.0.

### Step 7: The synthesis of 4-cyclopropyl-5-[8-imino-7-methyl-9-[[4-[1-methyl-4-(trifluoromethyl)imidazol-2-yl]phenyl]methyl]purin-2-yl]-6-methoxy-N-methyl-pyrimidin-2-amine

Formaldehyde (326 μmol, 24.4 μL, 37% wt. aq. soln., stab. with 7-8% methanol) was added to a solution of 4-cyclopropyl-5-[8-imino-7-methyl-9-[[4-[1-methyl-4-(trifluoromethyl)imidazol-2-yl]phenyl]methyl]purin-2-yl]-6-methoxy-pyrimidin-2-amine (199 mg, 296 μmol) in AcOH (3.0 mL) and EtOH (3 mL). The reaction mixture was stirred at room temperature for 4 hr. Sodium cyanoborohydride (27.9 mg, 445 μmol) was added to the reaction mixture portionwise. The resulting mixture was stirred at room temperature for 15 hr. The reaction mixture was subjected to HPLC (2-10 min, 40-95% ACN+FA, flow 30 ml/min; column SunFire C18 100×19 mm, 5 m) to afford 4-cyclopropyl-5-[8-imino-7-methyl-9-[[4-[1-methyl-4-(trifluoromethyl)imidazol-2-yl]phenyl]methyl]purin-2-yl]-6-methoxy-N-methyl-pyrimidin-2-amine (14.3 mg, 25.3 μmol, 8.55% yield) as an off-white solid.

¹H NMR (DMSO-d6, 600 MHz) δ 0.61-0.67 (m, 2H), 0.86-1.00 (m, 2H), 1.60-1.65 (m, 1H), 2.75 (s, 3H), 3.42 (s, 3H), 3.66-3.77 (m, 6H), 5.19 (s, 2H), 6.83-6.88 (m, 1H), 7.49 (d, 2H), 7.64 (d, 2H), 7.90 (s, 1H), 8.29-8.40 (m, 1H).

LCMS(ESI): [M+H]+ m/z: calcd 565.27; found 565.2.

### Example T-121

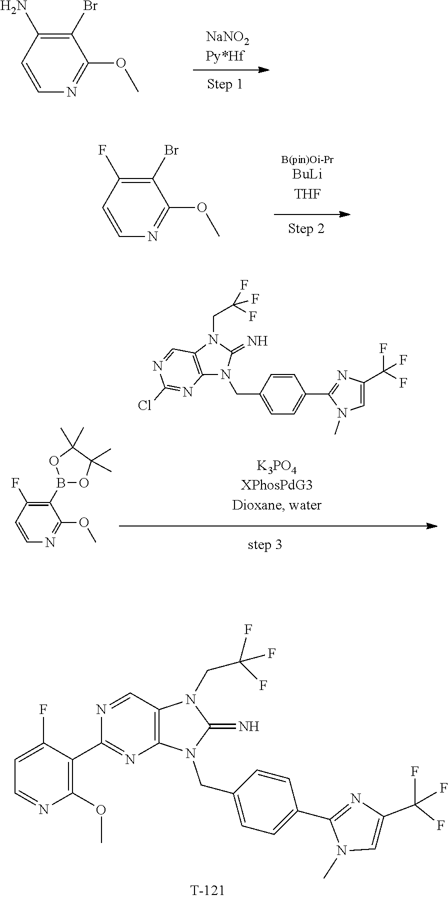

### Step 1: The synthesis of 3-bromo-4-fluoro-2-methoxy-pyridine

To a solution of 3-bromo-2-methoxy-pyridin-4-amine (2.00 g, 9.85 mmol) in Pyridine hydrofluoride (48.8 g, 493 mmol, 42.8 mL) Sodium Nitrite (1.02 g, 14.8 mmol) was added portionwise at 0° C. The resulting mixture was stirred at 0° C. for 1 hr. The reaction mixture was heated to 60° C. and stirred at this temperature for 12 hr. The mixture was cooled to room temperature, poured into ice-water (50 mL) and quenched with sat. aqueous solution NaHCO3 to pH-9-10. The resulting mixture was extracted EtOAc (2×20 mL). The combined organic layers were dried over anhydrous Na2SO4 and concentrated under reduced pressure. The residue was distillated at reduced pressure (bp.=75-80° C. at 20 mm Hg) to afford 3-bromo-4-fluoro-2-methoxy-pyridine (0.65 g, 3.16 mmol, 32.0% yield) as a yellow oil which was used in the next steps without further purification.

GCMS: [M−H]+ m/z: calcd 203.94, 205.94; found 204, 205.

### Step 2: The synthesis of 4-fluoro-2-methoxy-3-(4,4,5,5-tetramethyl-1,3,2-dioxaborolan-2-yl)pyridine

Butyl lithium (6.31 mmol, 2.63 mL, 2.4 M in hexane) was added dropwise to a stirred solution of 3-bromo-4-fluoro-2-methoxy-pyridine (650 mg, 3.16 mmol) and 2-isopropoxy-4,4,5,5-tetramethyl-1,3,2-dioxaborolane (881 mg, 4.73 mmol) in dry THE (10 mL) under argon atmosphere at −78° C. The reaction mixture was stirred for 4 h, during this time it was allowed to warm to 0° C. The mixture was quenched with sat. aqueous NH4Cl solution (2 mL). The obtained mixture was extracted with EtOAc (2×20 mL). The combined organic layers were washed with brine (5 mL) and concentrated under reduced pressure to afford 4-fluoro-2-methoxy-3-(4,4,5,5-tetramethyl-1,3,2-dioxaborolan-2-yl)pyridine (600 mg, 2.37 mmol, 75.1% yield) as a yellow oil which was used in the next steps without further purification.

GCMS: [M−H]+ m/z: calcd 253.13; found 253.1.

### Step 3: The synthesis of 2-(4-fluoro-2-methoxy-3-pyridyl)-9-[[4-[I-methyl-4-(trifluoromethyl)imidazol-2-yl]phenyl]methyl]-7-(2,2,2-trifluoroethyl)purin-8-imine

2-chloro-9-[[4-[1-methyl-4-(trifluoromethyl)imidazol-2-yl]phenyl]methyl]-7-(2,2,2-trifluoroethyl)purin-8-imine (0.13 g, 255 μmol), 4-fluoro-2-methoxy-3-(4,4,5,5-tetramethyl-1,3,2-dioxaborolan-2-yl)pyridine (484 mg, 383 μmol), Potassium phosphate tribasic anhydrous (135 mg, 638 μmol) and XPhos Pd G3 (10.8 mg, 12.8 μmol) were mixed in a degassed mixture of dioxane (5 mL) and water (0.5 mL). The mixture was stirred at 90° C. under argon atmosphere for 12 hr. The reaction mixture was cooled to room temperature. The reaction mixture was diluted with EtOAc (20 mL) and washed with water (10 mL) and brine (10 mL). To the obtained organic layer SiliaMetS® Dimercaptotriazine (20 mg) was added, and the mixture was stirred for 30 min. The mixture was filtered. The filtrate was concentrated under reduced pressure. The residue was subjected to HPLC (0.5-6.5 min, 28% water—ACN, +0.1% vol. of 25% aq. NH3, flow: 30 mL/min, column: XBridge C18 100×19 mm, 5 μm) to afford 2-(4-fluoro-2-methoxy-3-pyridyl)-9-[[4-[1-methyl-4-(trifluoromethyl)imidazol-2-yl]phenyl]methyl]-7-(2,2,2-trifluoroethyl)purin-8-imine (12.0 mg, 20.7 μmol, 8.10% yield) as a yellow solid.

¹H NMR (600 MHz, DMSO-d6) δ 3.73 (s, 3H), 3.82 (s, 3H), 4.77-4.98 (m, 2H), 5.08-5.20 (m, 2H), 7.04-7.12 (m, 2H), 7.47-7.52 (m, 2H), 7.64-7.70 (m, 2H), 7.90 (s, 1H), 8.23-8.27 (m, 1H), 8.28-8.35 (m, 1H).

LCMS(ESI): [M+H]+ m/z: calcd 581.19; found 581.2.

### Example T-097

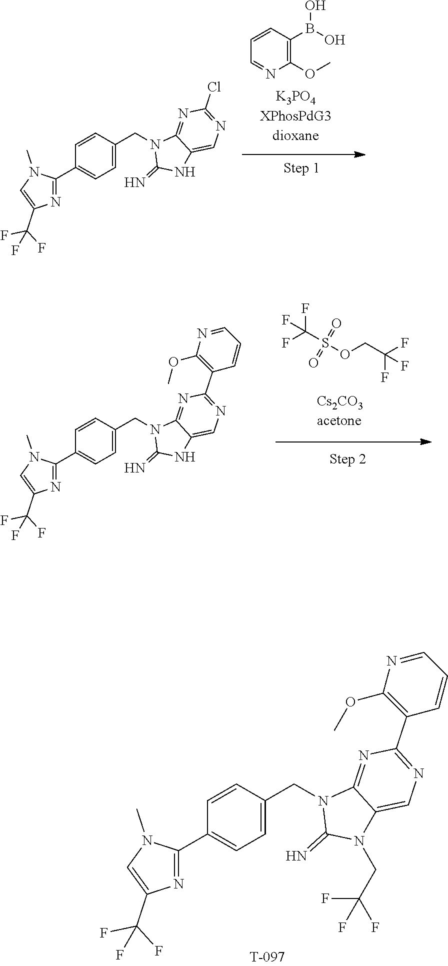 [SMILES: COc1ncccc1B(O)O ; Cn1cc(C(F)(F)F)nc1-c1ccc(Cn2c(=N)[nH]c3cnc(Cl)nc32)cc1 ; COc1ncccc1-c1ncc2[nH]c(=N)n(Cc3ccc(-c4nc(C(F)(F)F)cn4C)cc3)c2n1 ; O=S(=O)(OCC(F)(F)F)C(F)(F)F ; COc1ncccc1-c1ncc2c(n1)n(Cc1ccc(-c3nc(C(F)(F)F)cn3C)cc1)c(=N)n2CC(F)(F)F]

### Step 1: The synthesis of 2-(2-methoxy-3-pyridyl)-9-[[4-[1-methyl-4-(trifluoromethyl)imidazol-2-yl]phenyl]methyl]-7H-purin-8-imine

2-chloro-9-[[4-[1-methyl-4-(trifluoromethyl)imidazol-2-yl]phenyl]methyl]-7H-purin-8-imine (290 mg, 711 μmol), (2-methoxy-3-pyridyl)boronic acid (152 mg, 996 μmol) and potassium phosphate tribasic (453 mg, 2.13 mmol) were mixed in dioxane (25 mL). The resulting mixture was degassed. XPhosPdG3 (30.1 mg, 35.6 μmol) was added to the mixture. The reaction mixture was stirred at 100° C. for 16 hr. under argon atmosphere. The reaction mixture was cooled to room temperature, diluted with EtOAc (50 mL) and washed with water (2×20 mL).

SiliaMetS® Dimercaptotriazine (150 mg) was added to the organic layer. The resulting mixture was stirred for 30 min and filtered. The filtrate was concentrated under reduced pressure to afford 2-(2-methoxy-3-pyridyl)-9-[[4-[1-methyl-4-(trifluoromethyl)imidazol-2-yl]phenyl]methyl]-7H-purin-8-imine (300 mg, 624 μmol, 87.8% yield) as a yellow solid which was used in the next step without further purification.

LCMS(ESI): [M+H]+ m/z: calcd 481.19; found 481.2.

### Step 2: The synthesis of 2-(2-methoxy-3-pyridyl)-9-[[4-[1-methyl-4-(trifluoromethyl)imidazol-2-yl]phenyl]methyl]-7-(2,2,2-trifluoroethyl)purin-8-imine

2,2,2-trifluoroethyl trifluoromethanesulfonate (79.7 mg, 343 μmol, 49.5 μL) was added to a stirred mixture of 2-(2-methoxy-3-pyridyl)-9-[[4-[1-methyl-4-(trifluoromethyl)imidazol-2-yl]phenyl]methyl]-7H-purin-8-imine (150 mg, 312 μmol) and cesium carbonate (153 mg, 468 μmol) in acetone (10 mL). The reaction mixture was stirred at 50° C. for 16 hr. 2,2,2-trifluoroethyl trifluoromethanesulfonate (79.7 mg, 343 μmol, 49.5 μL) was added to the reaction mixture. The resulting mixture was stirred at 50° C. for 24 hr. The reaction mixture was cooled to room temperature and concentrated under reduced pressure. The residue was diluted with EtOAc (20 mL), washed with water (10 mL) and brine (10 mL). SiliaMetS® Dimercaptotriazine (60 mg) was added to the organic layer. The resulting mixture was stirred for 30 min and filtered. The filtrate was concentrated under reduced pressure. The residue was subjected to HPLC (0.5-6.5 min., 27% water—ACN, +0.1% vol. of 25% aq. NH3, flow: 30 mL/min, column: XBridge 100×19 mm, 5 μm), then repurified by HPLC (0.5-6.5 min., 40-90% water—MeOH, +0.1% vol. of 25% aq. NH3, flow: 30 mL/min, column: XBridge 100×19 mm, 5 μm), then repurified to HPLC (0.5-6.5 min., 28% water—ACN, +0.1% vol. of 25% aq. NH3, flow: 30 mL/min, column: XBridge 100×19 mm, 5 μm) to afford 2-(2-methoxy-3-pyridyl)-9-[[4-[1-methyl-4-(trifluoromethyl)imidazol-2-yl]phenyl]methyl]-7-(2,2,2-trifluoroethyl)purin-8-imine (5.50 mg, 9.78 μmol, 3.13% yield) as a white solid.

¹H NMR (DMSO-d6, 500 MHz) δ 3.74 (s, 3H), 3.86 (s, 3H), 4.78-5.02 (m, 2H), 5.06-5.25 (m, 2H), 7.00-7.11 (m, 2H), 7.54-7.60 (m, 2H), 7.65-7.72 (m, 2H), 7.90 (s, 1H), 7.95-7.99 (m, 2H), 8.22-8.24 (m, 1H), 8.26-8.33 (m, 1H).

LCMS(ESI): [M+H]+ m/z: calcd 563.20; found 563.2.

### Example T-110

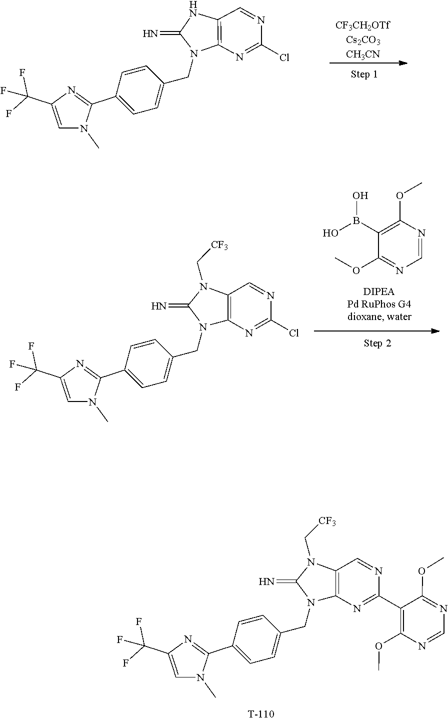

### Step 1: Synthesis of 2-chloro-9-(4-(1-methyl-4-(trifluoromethyl)-1H-imidazol-2-yl)benzyl)-7-(2,2,2-trifluoroethyl)-7H-purin-8(9H)-imine

Cesium carbonate (179 mg, 552 μmol) was added to a stirred solution of 2-chloro-9-[[4-[1-methyl-4-(trifluoromethyl)imidazol-2-yl]phenyl]methyl]-7H-purin-8-imine (150 mg, 368 mol) in ACN (8 mL) under argon atmosphere at 85° C. The mixture was stirred for 5 min, then 2,2,2-trifluoroethyl trifluoromethanesulfonate (128 mg, 552 μmol, 79.5 μL) was added. The reaction mixture was stirred at 85° C. for 18 hr. The reaction mixture was cooled to room temperature and concentrated under reduced pressure. The residue was dissolved in EtOAc (20 mL), washed with brine (2×10 mL), dried over anhydrous sodium sulfate and concentrated under reduced pressure. The residue was subjected to HPLC (2-10 min, 30% water+FA (0.1% vol.) -ACN+FA (0.1% vol.); flow: 30 mL/min, column: Waters SunFire C18, 100×19 mm, 5 μm) to afford 2-chloro-9-[[4-[1-methyl-4-(trifluoromethyl)imidazol-2-yl]phenyl]methyl]-7-(2,2,2-trifluoroethyl)purin-8-imine (29.0 mg, 59.2 μmol, 16.1% yield) as light-yellow solid.

LCMS(ESI): [M+H]+ m/z: calcd 490.12; found 490.0.

### Step 2: Synthesis of 2-(4,6-dimethoxypyrimidin-5-yl)-9-[[4-[1-methyl-4-(trifluoromethyl)imidazol-2-yl]phenyl]methyl]-7-(2,2,2-trifluoroethyl)purin-8-imine

2-Chloro-9-[[4-[1-methyl-4-(trifluoromethyl)imidazol-2-yl]phenyl]methyl]-7-(2,2,2-trifluoroethyl)purin-8-imine (29.0 mg, 59.2 μmol), (4,6-dimethoxypyrimidin-5-yl) boronic acid (10.0 mg, 59.2 μmol), Potassium phosphate tribasic (37.0 mg, 178 μmol) and RuPhos Pd G4 (2.5 mg, 3.0 μmol) were mixed in a degassed mixture of Dioxane (10 mL) and Water (2.5 mL) under inert atmosphere of argon. The reaction mixture was stirred at 95° C. for 16 hr. The mixture was cooled to room temperature and concentrated under reduced pressure. The residue was diluted with EtOAc (20 mL) and washed with brine (10 mL). Anhydrous sodium sulfate and SiliaMetS® Dimercaptotriazine (30 mg) were added to the mixture. The mixture was stirred for 1 hr. The solids were filtered out and washed with EtOAc. The filtrate was concentrated under reduced pressure and subjected to HPLC (2-10 min, 20-45% water—MeCN, +0.1% vol. of 25% aq. NH3, flow: 30 mL/min, column: SunFire C18 100×19 mm, 5 μm) to afford 2-(4,6-dimethoxypyrimidin-5-yl)-9-[[4-[1-methyl-4-(trifluoromethyl)imidazol-2-yl]phenyl]methyl]-7-(2,2,2-trifluoroethyl)purin-8-imine (15.7 mg, 26.5 μmol, 44.7% yield) as light-yellow solid.

¹H NMR (600 MHz, DMSO-d6) δ 3.74 (s, 3H), 3.84 (s, 6H), 4.75-5.01 (m, 2H), 5.04-5.22 (m, 2H), 6.99-7.09 (m, 1H), 7.46-7.52 (m, 2H), 7.64-7.70 (m, 2H), 7.90 (s, 1H), 8.24-8.31 (m, 1H), 8.55 (s, 1H).

LCMS (ESI): [M+H]+ m/z: calcd 594.20; found 594.4.

### Example T-100

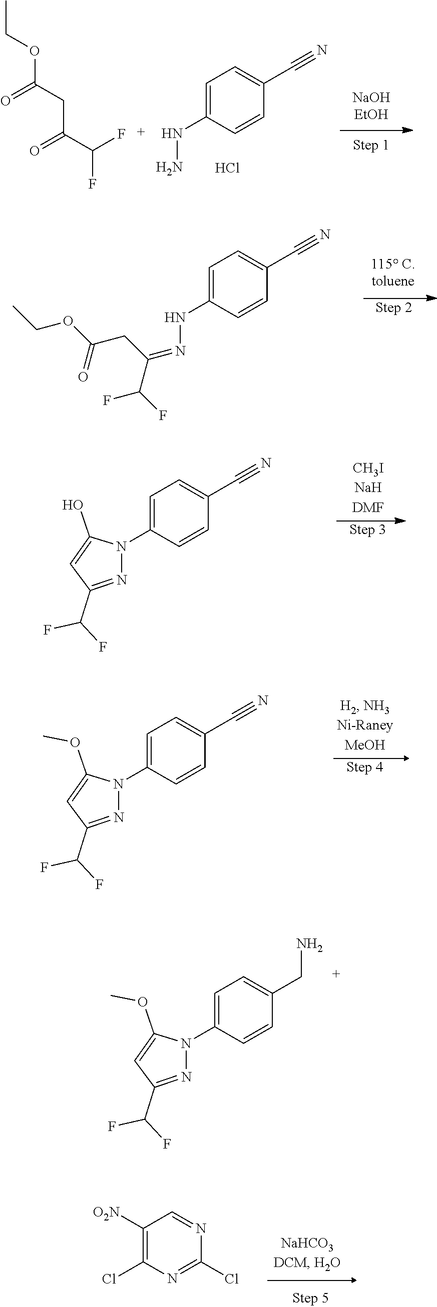

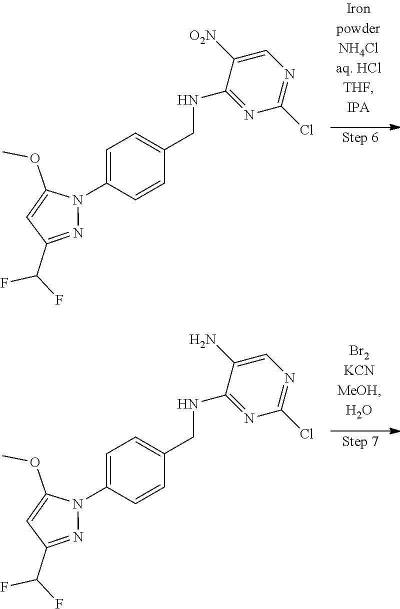 [SMILES: COc1cc(C(F)F)nn1-c1ccc(CNc2nc(Cl)ncc2[N+](=O)[O-])cc1 ; COc1cc(C(F)F)nn1-c1ccc(CNc2nc(Cl)ncc2N)cc1]

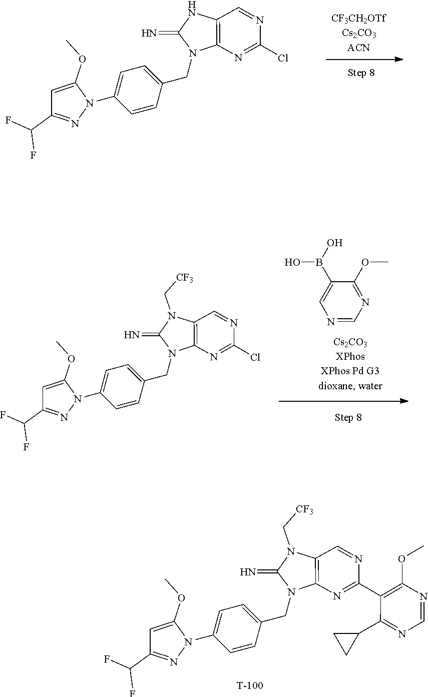 [SMILES: COc1cc(C(F)F)nn1-c1ccc(Cn2c(=N)[nH]c3cnc(Cl)nc32)cc1 ; COc1cc(C(F)F)nn1-c1ccc(Cn2c(=N)n(CC(F)(F)F)c3cnc(Cl)nc32)cc1 ; COc1ncncc1B(O)O ; COc1ncnc(C2CC2)c1-c1ncc2c(n1)n(Cc1ccc(-n3nc(C(F)F)cc3OC)cc1)c(=N)n2CC(F)(F)F]

### Step 1: The synthesis of ethyl 3-[(4-cyanophenyl)hydrazono]-4,4-difluoro-butanoate

To a solution of 4-hydrazinobenzonitrile (5.00 g, 29.5 mmol, HCl) in EtOH (100 mL) was added Sodium hydroxide (1.18 g, 29.5 mmol). The obtained mixture was stirred at room temperature for 40 min. To the resulting mixture ethyl 4,4-difluoro-3-oxo-butanoate (4.90 g, 29.5 mmol) in EtOH (20 mL) was added to the mixture. The resulting mixture was stirred at 80° C. for 12 hr. The reaction mixture was cooled to room temperature and filtered. The filtrate was concentrated in vacuo to afford ethyl 3-[(4-cyanophenyl)hydrazono]-4,4-difluoro-butanoate (5.00 g, 17.8 mmol, 60.3% yield) as an orange solid which was used in the next steps without further purification.

LCMS(ESI): [M+H]+ m/z: calcd 282.11; found 282.07

### Step 2: The synthesis of 4-[3-(difluoromethyl)-5-hydroxy-pyrazol-1-yl]benzonitrile

A solution of ethyl 3-[(4-cyanophenyl)hydrazono]-4,4-difluoro-butanoate (5.00 g, 17.8 mmol) and toluene-4-sulfonic acid (61.2 mg, 356 μmol) were dissolved in toluene (50 mL) and stirred at 115° C. for 3 hr. The mixture was cooled to room temperature and concentrated in vacuo to afford 4-[3-(difluoromethyl)-5-hydroxy-pyrazol-1-yl]benzonitrile (4.00 g, 17.0 mmol, 95.7% yield) as a brown solid which was used in the next steps without further purification.

¹H NMR (500 MHz, DMSO-d6) δ 5.79 (s, 1H), 6.88 (t, 1H, CHF2), 7.93-7.97 (m, 4H), 12.70 (br., 1H) LCMS(ESI): [M+H]+ m/z: calcd 236.07; found 235.0.

### Step 3: The synthesis of 4-[3-(difluoromethyl)-5-methoxy-pyrazol-1-yl]benzonitrile

Sodium hydride (896 mg, 23.4 mmol, 60% dispersion in mineral oil) was suspended in DMF (50 mL) at 0° C. 4-[3-(difluoromethyl)-5-hydroxy-pyrazol-1-yl]benzonitrile (5.00 g, 21.3 mmol) was added to the mixture at the same temperature followed by Iodomethane (3.02 g, 21.3 mmol, 1.32 mL). The resulting reaction mixture was stirred at room temperature for 12 hr. The solution was diluted with water (30 mL) and extracted with EtOAc (40 mL). The organic layer was washed with brine (30 mL), dried over anhydrous sodium sulfate and concentrated under reduced pressure. The residue was subjected to flash-column chromatography (SiO2, gradient hexane-MTBE) to afford 4-[3-(difluoromethyl)-5-methoxy-pyrazol-1-yl]benzonitrile (1.21 g, 4.90 mmol, 22.8% yield) as a white solid which was used in the next steps without further purification.

[symbol]

¹H NMR (400 MHz, CDCl3) δ 4.02 (s, 3H), δ 5.93 (s, 1H), 6.58 (t, 1H, CHF2), 7.72 (d, 2H), 7.90 (d, 2H).

LCMS(ESI): [M+H]+ m/z: calcd 250.23; found 250.00

### Step 4: The synthesis of [4-[3-(difluoromethyl)-5-methoxy-pyrazol-1-yl]phenyl]methanamine

A solution of 4-[3-(difluoromethyl)-5-methoxy-pyrazol-1-yl]benzonitrile (1.21 g, 4.90 mmol) in methanolic ammonia (20 mL, approx. 2-3% wt.) was hydrogenated under Ni-Raney (855 mg) at 50 bar at room temperature for 12 hr. The reaction mixture was filtered. The filtrate was concentrated in vacuo. The residue was redissolved in chloroform (50 mL). The resulting solution was filtered through a thin pad of silica gel. The filtrate was concentrated in vacuo to afford [4-[3-(difluoromethyl)-5-methoxy-pyrazol-1-yl]phenyl]methanamine (0.50 g, 2.00 mmol, 40.7% yield) as yellow oil which was used in the next steps without further purification.

¹H NMR (400 MHz, DMSO-d6) δ 1.85 (s, 2H), 3.74 (s, 2H), 3.96 (s, 3H), δ 6.20 (s, 1H), 6.78-7.06 (m, 1H), 7.44 (d, 2H), 7.54 (d, 2H)

LCMS(ESI): [M+H]+ m/z: calcd 254.11; found 254.05

### Step 5: The synthesis of 2-chloro-N-[[4-[3-(difluoromethyl)-5-methoxy-pyrazol-1-yl]phenyl]methyl]-5-nitro-pyrimidin-4-amine

A solution of Sodium bicarbonate (332 mg, 4.00 mmol) in water (10 mL) was added to a stirred solution of [4-[3-(difluoromethyl)-5-methoxy-pyrazol-1-yl]phenyl]methanamine (0.50 g, 2.00 mmol) in DCM (30 mL) at 0° C. To the resulting mixture a solution of 2,4-dichloro-5-nitro-pyrimidine (382 mg, 2.00 mmol) in DCM (10 mL) was added dropwise at 0° C. The resulting reaction mixture was stirred at 0° C. for 5.5 hr. The mixture was diluted with water (50 mL) and extracted with DCM (100 ml). The organic layer was washed with water (50 mL), dried over anhydrous Na2SO4 and concentrated under reduced pressure to afford 2-chloro-N-[[4-[3-(difluoromethyl)-5-methoxy-pyrazol-1-yl]phenyl]methyl]-5-nitro-pyrimidin-4-amine (0.63 g, 1.53 mmol, 77.7% yield) as yellow solid which was used in the next steps without further purification.

LCMS(ESI): [M+H]+ m/z: calcd 411.08; found 411.0.

### Step 6: The synthesis of 2-chloro-N4-[[4-[3-(difluoromethyl)-5-methoxy-pyrazol-1-yl]phenyl]methyl]pyrimidine-4,5-diamine

Iron powder (545.36 mg, 9.76 mmol, 69.38 μL) was added portionwise to a stirred mixture of 2-chloro-N-[[4-[3-(difluoromethyl)-5-methoxy-pyrazol-1-yl]phenyl]methyl]-5-nitro-pyrimidin-4-amine (573 mg, 1.39 mmol) and Ammonium Chloride (746 mg, 14.0 mmol) in THE (15 mL) and IPA (15 mL) followed by Hydrochloric acid (590 μL, 36% wt. aq. soln.). The reaction mixture was stirred at 45° C. for 18 hr. The reaction mixture was cooled to room temperature and filtered. The filter cake was washed with IPA (20 mL). The combined filtrates were concentrated under reduced pressure. The residue was quenched with aq. soln. of potassium carbonate (20 mL, 10% wt.). The resulting mixture was extracted with EtOAc (60 mL). The organic layer was washed with brine (30 mL), dried over anhydrous sodium sulfate and concentrated under reduced pressure to afford 2-chloro-N4-[[4-[3-(difluoromethyl)-5-methoxy-pyrazol-1-yl]phenyl]methyl]pyrimidine-4,5-diamine (496 mg, 1.30 mmol, 93.4% yield) as a brown solid.

LCMS(ESI): [M+H]+ m/z: calcd 381.11; found 381.2.

### Step 7: The synthesis of 2-chloro-9-[[4-[3-(difluoromethyl)-5-methoxy-pyrazol-1-yl]phenyl]methyl]-7H-purin-8-imine

To a stirred solution of molecular bromine (2.42 g, 15.1 mmol) in MeOH (10 mL) a solution of Potassium cyanide (985.06 mg, 15.13 mmol) in water (2 mL) was added at 0° C. The mixture was stirred at 0° C. for 15 min. A solution of 2-chloro-N4-[[4-[3-(difluoromethyl)-5-methoxy-pyrazol-1-yl]phenyl]methyl]pyrimidine-4,5-diamine (576 mg, 1.50 mmol) in MeOH (3 mL) was added to the mixture in one portion. The resulting mixture was stirred for 16 hr. at room temperature. The solvents were removed by evaporation in vacuo. The residue was partitioned between EtOAc (80 mL) and 10% aq. soln. of potassium carbonate (50 mL). The organic layer was dried over anhydrous sodium sulfate, filtered and concentrated under reduced pressure to afford 2-chloro-9-[[4-[3-(difluoromethyl)-5-methoxy-pyrazol-1-yl]phenyl]methyl]-7H-purin-8-imine (560 mg, 1.4 mmol, 91.23% yield) as a brown oil which was used in the next steps without further purification.

LCMS(ESI): [M+H]+ m/z: calcd 406.10; found 406.09 Step 8: The synthesis of 2-chloro-9-[[4-[3-(difluoromethyl)-5-methoxy-pyrazol-1-yl]phenyl]methyl]-7-(2,2,2-trifluoroethyl)purin-8-imine

2,2,2-trifluoroethyl trifluoromethanesulfonate (412 mg, 1.8 mmol, 256 μL) was added dropwise to a stirred mixture of 2-chloro-9-[[4-[3-(difluoromethyl)-5-methoxy-pyrazol-1-yl]phenyl]methyl]-7H-purin-8-imine (480 mg, 1.20 mmol) and Cesium carbonate (771 mg, 2.40 mmol) in ACN (10 mL). The resulting mixture was stirred at 60° C. for 16 hr. The reaction mixture was concentrated under reduced pressure. The residue was diluted with water (30 mL) and extracted with EtOAc (40 mL). The organic layer was washed with brine (30 mL), dried over anhydrous sodium sulfate and concentrated under reduced pressure. The residue was subjected to HPLC (0-1-6 min, 20-50% water+FA (0.1% vol.)—ACN+FA (0.1% vol.); flow: 30 mL/min, column: Chromatorex 18 SMB100-5T 100×19 mm, 5 m) to afford 2-chloro-9-[[4-[3-(difluoromethyl)-5-methoxy-pyrazol-1-yl]phenyl]methyl]-7H-purin-8-imine (76.0 mg, 156 μmol, 13.2% yield) as an yellow solid.

LCMS(ESI): [M+H]+ m/z: calcd 488.12; found 488.0.

### Step 8: The synthesis of 2-(4-cyclopropyl-6-methoxy-pyrimidin-5-yl)-9-[[4-[3-(difluoromethyl)-5-methoxy-pyrazol-1-yl]phenyl]methyl]-7-(2,2,2-trifluoroethyl)purin-8-imine

2-chloro-9-[[4-[3-(difluoromethyl)-5-methoxy-pyrazol-1-yl]phenyl]methyl]-7-(2,2,2-trifluoroethyl)purin-8-imine (75.0 mg, 154 μmol), (4-cyclopropyl-6-methoxy-pyrimidin-5-yl)boronic acid (59.7 mg, 307 μmol), cesium carbonate (150 mg, 461 μmol) and XPhos Pd G3 (6.51 mg, 7.69 μmol) were mixed in degassed mixture of Dioxane (4 mL) and Water (1 mL) under inert atmosphere of argon. The reaction mixture was stirred at 95° C. for 16 hr. The reaction mixture was cooled to room temperature, concentrated under reduced pressure, diluted with EtOAc (20 mL) and washed with brine (10 mL). The organic layer was dried over sodium sulfate, filtered and concentrated. The residue was subjected to HPLC (0-1-6 min, 40-80% water -MeOH, +0.1% vol. of 25% aq. NH3, flow: 30 mL/min, column: XBridge BEH C18 100×19 mm, 5 m) to afford 2-(4-cyclopropyl-6-methoxy-pyrimidin-5-yl)-9-[[4-[3-(difluoromethyl)-5-methoxy-pyrazol-1-yl]phenyl]methyl]-7-(2,2,2-trifluoroethyl)purin-8-imine (35.7 mg, 59.4 μmol, 38.6% yield) as yellow solid.

¹H NMR (500 MHz, DMSO-d6) δ 0.77-0.84 (m, 2H), 0.96-1.02 (m, 2H), 1.63-1.70 (m, 1H), 3.81 (s, 3H), 3.93 (s, 3H), 4.75-5.02 (m, 2H), 5.04-5.22 (m, 2H), 6.19 (s, 1H), 6.90 (t, 1H, CHF2), 7.04-7.12 (m, 1H), 7.47 (d, 2H), 7.52-7.59 (m, 2H), 8.28-8.36 (m, 1H), δ 8.62 (s, 1H)

LCMS(ESI): [M+H]+ m/z: calcd 602.23; found 602.00.

### Example T-115

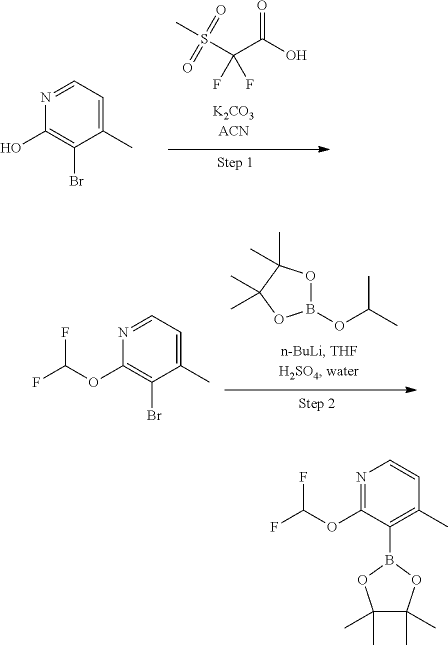

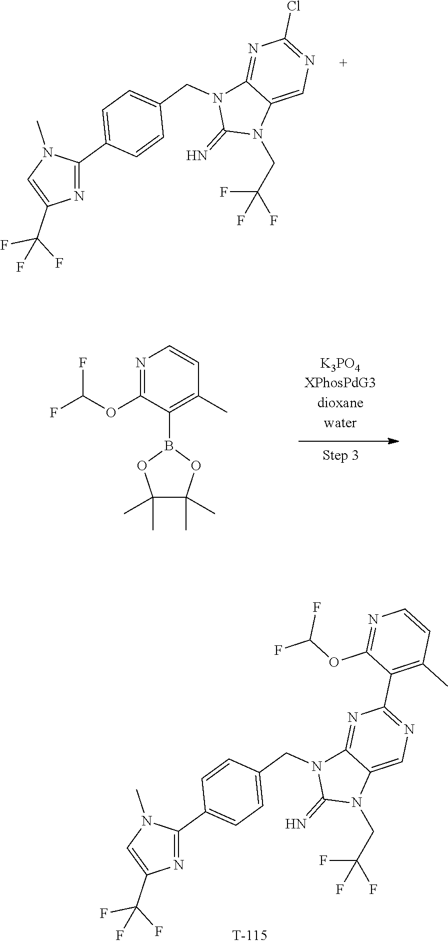 [SMILES: Cn1cc(C(F)(F)F)nc1-c1ccc(Cn2c(=N)n(CC(F)(F)F)c3cnc(Cl)nc32)cc1 ; Cc1ccnc(OC(F)F)c1B1OC(C)(C)C(C)(C)O1 ; Cc1ccnc(OC(F)F)c1-c1ncc2c(n1)n(Cc1ccc(-c3nc(C(F)(F)F)cn3C)cc1)c(=N)n2CC(F)(F)F]

### Step 1: The synthesis of 3-bromo-2-(difluoromethoxy)-4-methyl-pyridine

2,2-difluoro-2-fluorosulfonyl-acetic acid (1.04 g, 5.85 mmol) and potassium carbonate (1.52 g, 11.0 mmol) were added to a stirred solution of 3-bromo-4-methyl-pyridin-2-ol (1.00 g, 5.32 mmol) in ACN (5.0 mL). The reaction mixture was stirred at room temperature for 12 hr under argon atmosphere. The reaction mixture was quenched by addition of water (10 mL) and extracted with MTBE (20 mL). The organic layer was separated, washed with brine (10 mL) dried over anhydrous sodium sulphate and concentrated under reduced pressure to afford 3-bromo-2-(difluoromethoxy)-4-methyl-pyridine (1.20 g, 5.04 mmol, 94.8% yield) as a light-yellow oil which was used in the next steps without further purification.

¹H NMR (600 MHz, DMSO-d6) δ 2.43 (s, 3H), 6.95 (d, 1H), 7.42 (t, 1H, CHF2), 7.93 (d, 1H).

### Step 2: The synthesis of 2-(difluoromethoxy)-4-methyl-3-(4,4,5,5-tetramethyl-1,3,2-dioxaborolan-2-yl)pyridine

n-Butyllithium (5.76 mmol, 2.4 mL, 2.5M in hexane) was added dropwise to a precooled to −78° C. solution of 3-bromo-2-(difluoromethoxy)-4-methyl-pyridine (685 mg, 2.88 mmol) and 2-isopropoxy-4,4,5,5-tetramethyl-1,3,2-dioxaborolane (1.07 g, 5.76 mmol) in THE (9.50 mL) under argon atmosphere. The reaction mixture was stirred at −78° C. for 4 hr. The reaction mixture was quenched by addition of a saturated aqueous solution of NH4Cl (25 mL) and extracted with EtOAc (30 mL). The organic layer was separated, washed with water (20 mL), dried over anhydrous sodium sulfate and concentrated under reduced pressure to afford 2-(difluoromethoxy)-4-methyl-3-(4,4,5,5-tetramethyl-1,3,2-dioxaborolan-2-yl)pyridine (800 mg, crude) as a light-yellow oil which was used in the next steps without further purification.

GCMS(ESI): [M]+ m/z: calcd 285.13; found 285

### Step 3: The synthesis of methyl 2-(2-(difluoromethoxy)-4-methylpyridin-3-yl)-9-(4-(1-methyl-4-(trifluoromethyl)-1H-imidazol-2-yl)benzyl)-7-(2,2,2-trifluoroethyl)-7H-purin-8(9H)-imine

2-Chloro-9-(4-(1-methyl-4-(trifluoromethyl)-1H-imidazol-2-yl)benzyl)-7-(2,2,2-trifluoroethyl)-7H-purin-8(9H)-imine (50.0 mg, 102 μmol), 2-(difluoromethoxy)-4-methyl-3-(4,4,5,5-tetramethyl-1,3,2-dioxaborolan-2-yl)pyridine (58.2 mg, 204 μmol), potassium phosphate tribasic anhydrous (65.0 mg, 306 μmol) and XPhos Pd G3 (6.05 mg, 7.15 μmol) were mixed in a degassed mixture of dioxane (4.0 mL) and water (0.4 mL) under argon atmosphere. The reaction mixture was stirred at 85° C. for 12 hr. The reaction mixture was cooled to room temperature. SiliaMetS® Dimercaptotriazine (100 mg) was added to the reaction mixture. The resulting mixture was stirred at room temperature for 3 hr. The mixture was diluted with MTBE (5.0 mL) and filtered through a pad of silica gel. The filtrate was concentrated under reduced pressure. The residue was subjected to HPLC (2-10 min, 20-45% ACN+FA (0.1% vol.); flow: 30 mL/min, column: SunFire C18, 100×19 mm, 5 μm) to afford 2-(2-(difluoromethoxy)-4-methylpyridin-3-yl)-9-(4-(1-methyl-4-(trifluoromethyl)-1H-imidazol-2-yl)benzyl)-7-(2,2,2-trifluoroethyl)-7H-purin-8(9H)-imine (9.50 mg, 15.5 μmol, 15.2% yield) as a yellow solid.

¹H NMR (600 MHz, DMSO-d6) δ 2.08 (s, 3H), 3.72 (s, 3H), 4.77-5.05 (m, 2H), 5.06-5.24 (m, 2H), 7.05-7.14 (m, 1H), 7.22 (d, 1H), 7.50 (d, 2H), 7.64 (d, 2H), 7.68 (t, 1H, CHF2), 7.90 (s, 1H), 8.16 (d, 1H), 8.31-8.38 (m, 1H).

LCMS(ESI): [M+H]+ m/z: calcd 613.20; found 613.2.

### Example T-135

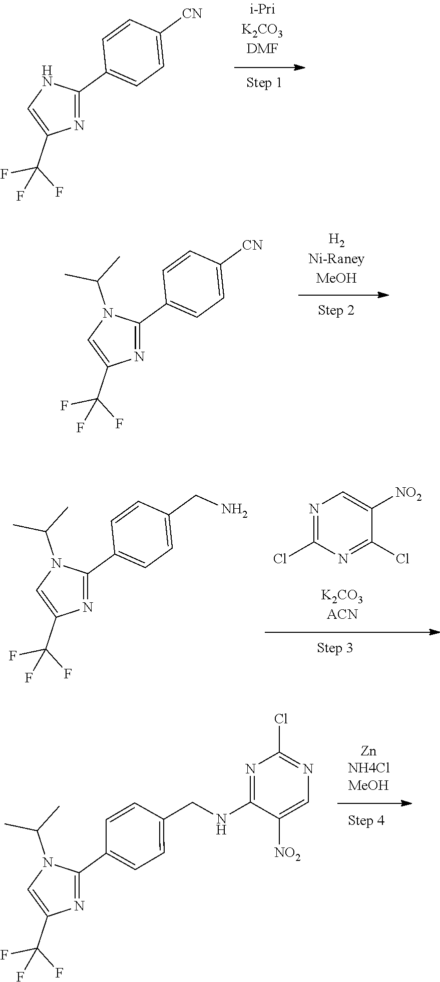 [SMILES: N#Cc1ccc(-c2nc(C(F)(F)F)c[nH]2)cc1 ; CC(C)n1cc(C(F)(F)F)nc1-c1ccc(C#N)cc1 ; CC(C)n1cc(C(F)(F)F)nc1-c1ccc(CN)cc1 ; O=[N+]([O-])c1cnc(Cl)nc1Cl ; CC(C)n1cc(C(F)(F)F)nc1-c1ccc(CNc2nc(Cl)ncc2[N+](=O)[O-])cc1]

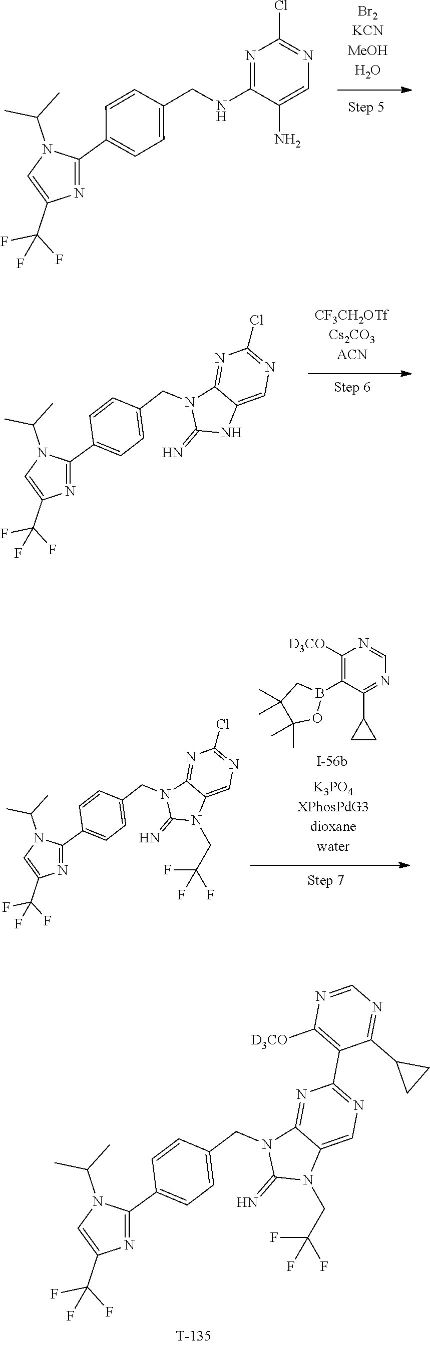

### Step 1: The synthesis of 4-[1-isopropyl-4-(trifluoromethyl)imidazol-2-yl]benzonitrle

4-[4-(trifluoromethyl)-1H-imidazol-2-yl]benzonitrile (6.00 g, 25.3 mmol), potassium carbonate (8.74 g, 63.2 mmol) and 2-iodopropane (12.9 g, 75.9 mmol, 7.58 mL) were mixed in DMF (100 mL) at room temperature. The resulting mixture was stirred at 90° C. for 72 hr. The mixture was cooled to room temperature and poured into ice-cold water (200 mL). The resulting mixture was extracted with EtOAc (300 mL). The organic layer was washed with water (100 mL) and brine (100 mL) and concentrated under reduced pressure to afford 4-[1-isopropyl-4-(trifluoromethyl)imidazol-2-yl]benzonitrile (5.00 g, 17.9 mmol, 70.8% yield) as a yellow solid which was used in the next steps without further purification.

¹H NMR (500 MHz, DMSO-d6) δ 1.43 (d, 6H), 4.47-4.55 (m, 1H), 7.80 (d, 2H), 8.01 (d, 2H), 8.27 (s, 1H).

LCMS(ESI): [M+H]+ m/z: calcd 280.13; found 280.0.

### Step 2: The synthesis of[4-[I-isopropyl-4-(trfluoromethyl)imidazol-2-yl]phenyl]methanamin

Ni-Raney (700 mg) was added to a solution of 4-[1-isopropyl-4-(trifluoromethyl)imidazol-2-yl]benzonitrile (5.00 g, 17.9 mmol) in MeOH (400 mL). The resulting mixture was subjected to hydrogenation at 40 atm for 12 hr. The reaction mixture was filtered through a thin pad of silica. The filtrate was concentrated under reduced pressure. The residue was redissolved in DCM (100 mL). The resulting solution was dried over anhydrous sodium sulfate and filtered. The filtrate was concentrated under reduced pressure to afford [4-[1-isopropyl-4-(trifluoromethyl)imidazol-2-yl]phenyl]methanamine (5.00 g, 17.7 mmol, 98.6% yield) as a brown solid which was used in the next steps without further purification.

¹H NMR (400 MHz, DMSO-d6) δ 1.40 (d, 6H), 3.81 (s, 3H), 4.42-4.53 (m, 1H), 7.49 (s, 4H), 8.16 (s, 1H).

LCMS(ESI): [M+H]+ m/z: calcd 284.17; found 284.0.

### Step 3: The synthesis of 2-chloro-N-[[4-[1-isopropyl-4-(trifluoromethyl)imidazol-2-yl]phenyl]methyl]-5-nitro-pyrimidin-4-amine

2,4-Dichloro-5-nitro-pyrimidine (3.57 g, 18.4 mmol) and potassium carbonate (3.66 g, 26.5 mmol) were added to a solution of [4-[1-isopropyl-4-(trifluoromethyl)imidazol-2-yl]phenyl]methanamine (5.00 g, 17.7 mmol) was in ACN (100 mL). The reaction mixture was stirred at room temperature for 18 hr. The reaction mixture was concentrated under reduced pressure. The residue was diluted with water (100 mL) and extracted with EtOAc (3×50 mL). Combined organic layers were dried over anhydrous sodium sulfate and concentrated under reduced pressure to afford 2-chloro-N-[[4-[1-isopropyl-4-(trifluoromethyl)imidazol-2-yl]phenyl]methyl]-5-nitro-pyrimidin-4-amine (7.00 g, 15.9 mmol, 90.0% yield) as a brown solid which was used in the next steps without further purification.

LCMS(ESI): [M+H]+ m/z: calcd 441.12; found 441.2.

### Step 4: The synthesis of 2-chloro-N4-[[4-[1-isopropyl-4-(trifluoromethyl)imidazol-2-yl]phenyl]methyl]pyrimidine-4,5-diamine

Ammonium chloride (12.7 g, 238 mmol) and zinc powder (6.23 g, 95.28 mmol) were added sequentially to a solution of 2-chloro-N-[[4-[1-isopropyl-4-(trifluoromethyl)imidazol-2-yl]phenyl]methyl]-5-nitro-pyrimidin-4-amine (7.00 g, 15.9 mmol) in MeOH (200 mL) at 0° C. The resulting mixture was stirred at ambient temperature for 18 hr. The reaction mixture was filtered. The filtrate was concentrated under reduced pressure. The residue was diluted with water (200 mL) and extracted with EtOAc (3×100 mL). The combined organic layers were dried over anhydrous sodium sulfate and concentrated under reduced pressure to afford 2-chloro-N4-[[4-[1-isopropyl-4-(trifluoromethyl)imidazol-2-yl]phenyl]methyl]pyrimidine-4,5-diamine (6.00 g, 14.6 mmol, 92.0% yield) as a brown solid which was used in the next steps without further purification.

LCMS(ESI): [M+H]+ m/z: calcd 411.16; found 411.2.

### Step 5: The synthesis of 2-chloro-9-[[4-[1-isopropyl-4-(trifluoromethyl)imidazol-2-yl]phenyl]methyl]-7H-purin-8-imin

A solution of potassium cyanide (6.75 g, 104 mmol) in water (20 mL) was added dropwise to a solution of bromine (16.4 g, 103 mmol) in MeOH (200 mL) at 0° C. The resulting mixture was stirred at 0° C. for 15 min. The solution of 2-chloro-N4-[[4-[1-isopropyl-4-(trifluoromethyl)imidazol-2-yl]phenyl]methyl]pyrimidine-4,5-diamine (6.00 g, 14.6 mmol) in MeOH (20 mL) was added in one portion to the mixture. The resulting mixture was stirred at ambient for 18 hr. The reaction mixture was diluted with water (200 mL) and extracted with EtOAc (3×100 mL). The combined organic layers were dried over anhydrous sodium sulfate and concentrated under reduced pressure. The residue was subjected to flash-column chromatography (SiO2, gradient MTBE—methanol) to afford 2-chloro-9-[[4-[1-isopropyl-4-(trifluoromethyl)imidazol-2-yl]phenyl]methyl]-7H-purin-8-imine (3.80 g, 8.7 mmol, 59.7% yield) as a yellow solid.

LCMS(ESI): [M+H]+ m/z: calcd 436.15; found 436.2.

### Step 6: The synthesis of 2-chloro-9-[[4-[1-isopropyl-4-(trifluoromethyl)imidazol-2-yl]phenyl]methyl]-7-(2,2,2-trifluoroethyl)purin-8-imine

2,2,2-Trifluoroethyl trifluoromethanesulfonate (1.04 g, 4.47 mmol, 644 μL) was added to a mixture of 2-chloro-9-[[4-[1-isopropyl-4-(trifluoromethyl)imidazol-2-yl]phenyl]methyl]-7H-purin-8-imine (1.30 g, 2.98 mmol) and cesium carbonate (2.43 g, 7.46 mmol) in ACN (60 mL). The reaction mixture was stirred at 80° C. for 96 hr. The reaction mixture was cooled to room temperature, diluted with water (100 mL) and extracted with EtOAc (3×45 mL). Combined organic layers were dried over anhydrous sodium sulfate and concentrated under reduced pressure. The residue was subjected to flash-column chromatography (SiO2, EtOAc—Hex 9:1) to afford a mixture of desired product with isomeric 2-chloro-9-[[4-[1-isopropyl-4-(trifluoromethyl)imidazol-2-yl]phenyl]methyl]-N-(2,2,2-trifluoroethyl)purin-8-amine. The mixture was subjected to HPLC (2-10 min, 0-55% water+FA (0.1% vol.)—ACN+FA (0.1% vol.); flow: 30 mL/min, column: Waters SunFire C18, 100×19 mm, 5 μm) to afford 2-chloro-9-[[4-[1-isopropyl-4-(trifluoromethyl)imidazol-2-yl]phenyl]methyl]-7-(2,2,2-trifluoroethyl)purin-8-imine (100 mg, 193 μmol, 6.47% yield) and 2-chloro-9-[[4-[1-isopropyl-4-(trifluoromethyl)imidazol-2-yl]phenyl]methyl]-N-(2,2,2-trifluoroethyl)purin-8-amine (250 mg, 483 μmol, 16.2% yield) as yellow solids.

LCMS(ESI): [M+H]+ m/z: calcd 518.16; found 518.2.

### Step 7: The synthesis of 2-[4-cyclopropyl-6-(trideuteriomethoxy)pyrimidin-5-yl]-9-[[4-[1-isopropyl-4-(trifluoromethyl)imidazol-2-yl]phenyl]methyl]-7-(2,2,2-trifluoroethyl)purin-8-imine

The synthesis of the starting 4-cyclopropyl-5-(4,4,5,5-tetramethyl-1,3,2-dioxaborolan-2-yl)-6-(trideuteriomethoxy)pyrimidine is described by Intermediate 56. [1300] 2-Chloro-9-[[4-[1-isopropyl-4-(trifluoromethyl)imidazol-2-yl]phenyl]methyl]-7-(2,2,2-trifluoroethyl)purin-8-imine (34.5 mg, 67.2 μmol), 4-cyclopropyl-5-(4,4,5,5-tetramethyl-1,3,2-dioxaborolan-2-yl)-6-(trideuteriomethoxy)pyrimidine (75.0 mg, 269 μmol), potassium phosphate tribasic anhydrous (42.8 mg, 202 μmol) and XPhosPdG3 (5.69 mg, 6.72 μmol) were mixed in a degassed mixture of dioxane (600 μL) and water (100 μL). The reaction mixture was stirred at 80° C. for 12 hr. The reaction mixture was cooled to room temperature, diluted with EtOAc (15 mL) and washed with water (5.0 mL). The organic layer was separated, washed with brine (2×10 mL), dried over anhydrous sodium sulfate and concentrated under reduced pressure. The residue was subjected to HPLC (0-2-10 min., 28-35-70% water—ACN, flow: 30 mL/min, column: Chromatorex C18 SMB100-5T, 100×19 mm, 5 μm) to afford 2-[4-cyclopropyl-6-(trideuteriomethoxy)pyrimidin-5-yl]-9-[[4-[1-isopropyl-4-(trifluoromethyl)imidazol-2-yl]phenyl]methyl]-7-(2,2,2-trifluoroethyl)purin-8-imine (12.0 mg, 18.9 μmol, 28.2% yield) as a light-yellow solid.

¹H NMR (600 MHz, DMSO-d6) δ 0.80-0.85 (m, 2H), 0.98-1.03 (m, 2H), 1.39 (d, 2H), 1.67-1.73 (m, 1H), 4.39-4.45 (m, 1H), 4.79-5.04 (m, 2H), 5.10-5.27 (m, 2H), 7.08-7.13 (m, 1H), 7.49-7.55 (m, 4H), 8.16 (s, 1H), 8.32-8.38 (m, 1H), 8.63 (s, 1H).

LCMS(ESI): [M+H]+ m/z: calcd 635.25; found 635.2.

### Example T-105

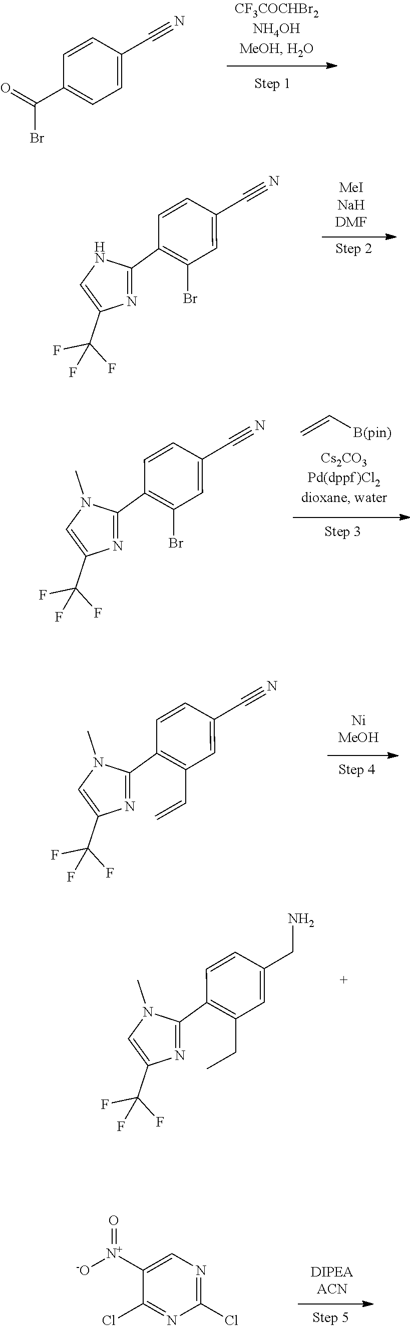

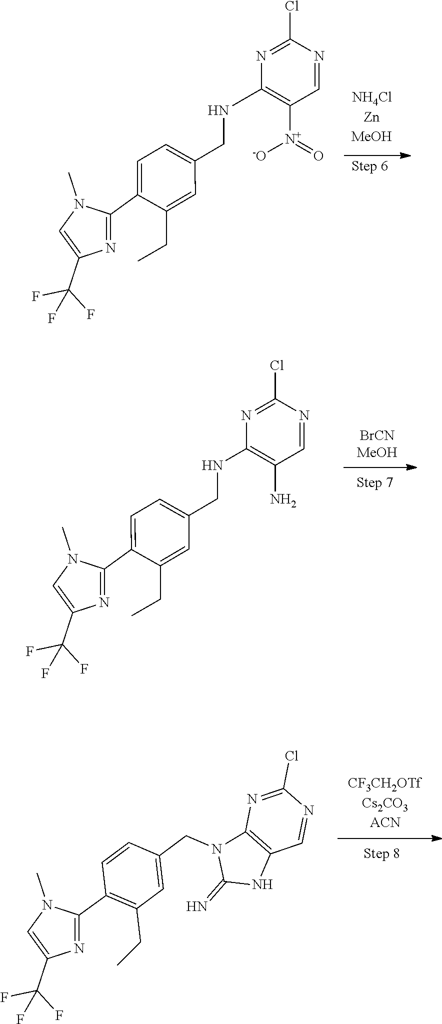

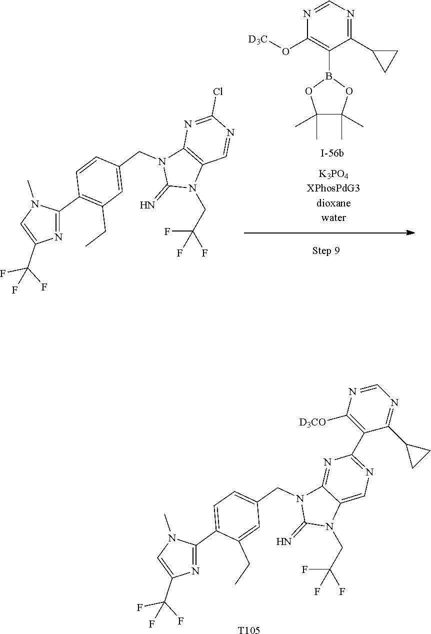

### Step 1: The synthesis of 3-bromo-4-[4-(trifluoromethyl)-1H-imidazol-2-yl]benzonitrile

A mixture of 3,3-dibromo-1,1,1-trifluoro-propan-2-one (8.48 g, 31.42 mmol) and sodium acetate (5.39 g, 65.7 mmol) in water (25 mL) was stirred at 100° C. for 45 min. The mixture was cooled to room temperature and added to a solution of 3-bromo-4-formyl-benzonitrile (6.00 g, 28.6 mmol) and ammonium hydroxide (30 mL, 25% wt. in water) in MeOH (160 mL). The reaction mixture was stirred for 40 min at room temperature, then at 80° C. for 2 hr. The reaction mixture was cooled to room temperature and concentrated under the reduced pressure to approximately 50 mL. The precipitate formed was filtered off, washed with water and dried under reduced pressure. The residue was re-dissolved in EtOAc (300 mL). The resulting organic solution was washed with brine (2×30 mL), dried over anhydrous Na2SO4 and concentrated under reduced pressure. The residue was subjected to flash-column chromatography (SiO2, gradient chloroform—acetonitrile) to afford 3-bromo-4-[4-(trifluoromethyl)-1H-imidazol-2-yl]benzonitrile (3.00 g, 9.49 mmol, 33.2% yield) as a yellow solid.

¹H NMR (400 MHz, DMSO-d6) δ 7.82 (d, 1H), 7.99 (d, 1H), 8.02 (s, 1H), 8.37 (s, 1H), 13.22 (br., 1H).

LCMS(ESI): [M+H]+ m/z: calcd 315.97; found 315.9.

### Step 2: The synthesis of 3-bromo-4-[I-methyl-4-(trifluoromethyl)imidazol-2-yl]benzonitrile

3-bromo-4-[4-(trifluoromethyl)-1H-imidazol-2-yl]benzonitrile (5.00 g, 15.8 mmol) was dissolved in DMF (60 mL). The solution was cooled to 0° C. then Sodium hydride (696 mg, 17.4 mmol, 60% dispersion in mineral oil) was added in few portions. The reaction mixture was stirred at room temperature for 2 hr. The mixture was cooled to 0° C. then methyl iodide (2.58 g, 18.2 mmol, 1.13 mL) was added in one portion. The reaction mixture was stirred at room temperature for 16 hr. The mixture was poured into ice-water mixture (400 mL). The solid precipitate formed was filtered off and redissolved in EtOAc (300 mL). The resulting solution was washed with brine (100 mL), dried over anhydrous Na2SO4 and concentrated under reduced pressure to afford 3-bromo-4-[1-methyl-4-(trifluoromethyl)imidazol-2-yl]benzonitrile (4.00 g, 12.1 mmol, 76.6% yield) as a yellow solid which was used in the next steps without further purification.

¹H NMR (400 MHz, DMSO-d6) δ 3.51 (s, 3H), 7.75 (d, 1H), 8.00-8.05 (m, 2H), 8.42 (s, 1H).

LCMS(ESI): [M+H]+ m/z: calcd 329.99; found 330.0.

### Step 3: The synthesis of 4-[I-methyl-4-(trifluoromethyl)imidazol-2-yl]-3-vinyl-benzonitrile

3-Bromo-4-[1-methyl-4-(trifluoromethyl)imidazol-2-yl]benzonitrile (3.00 g, 9.09 mmol), 4,4,5,5-tetramethyl-2-vinyl-1,3,2-dioxaborolane (2.80 g, 18.2 mmol), cesium carbonate (7.40 g, 22.7 mmol) and bis(diphenylphosphino)ferrocene]dichloropalladium(II)-DCM (332 mg, 454 mol) were mixed in a degassed mixture of dioxane (60 mL) and water (3 mL) under argon atmosphere. The mixture was stirred at 65° C. for 48 hr. Aliquot showed the presence of the product (49%) and the starting bromide (29%) according to LCMS. The solution was stirred at 65° C. for another 48 hr. The mixture was cooled to room temperature and concentrated under reduced pressure. The residue was partioned between EtOAc (150 mL) and water (50 mL). The organic layer was separated, washed with brine (2×50 mL), dried over anhydrous sodium sulfate and concentrated under reduced pressure to afford 4-[1-methyl-4-(trifluoromethyl)imidazol-2-yl]-3-vinyl-benzonitrile (3.00 g, crude) as a brown solid which was used in the next steps without further purification.

LCMS(ESI): [M+H]+ m/z: calcd 278.09; found 278.2.

### Step 4: The synthesis of [3-ethyl-4-[1-methyl-4-(trifluoromethyl)imidazol-2-yl]phenyl]methanamine

Raney-nickel (220 mg) was added to a solution of 4-[1-methyl-4-(trifluoromethyl)imidazol-2-yl]-3-vinyl-benzonitrile (2.00 g, crude) in MeOH (200 mL). The reaction mixture was hydrogenated at 50 atm at room temperature for 48 hr. The mixture was filtered. The filtrate was concentrated under reduced pressure to afford [3-ethyl-4-[1-methyl-4-(trifluoromethyl)imidazol-2-yl]phenyl]methanamine (2.10 g, crude) as a brown oil which was used in the next steps without further purification.

¹H NMR (400 MHz, DMSO-d6) δ 0.98 (t, 3H), 2.50-2.57 (m, 2H), 3.46 (s, 3H), 3.76 (s, 2H), 7.23-7.31 (m, 2H), 7.36 (s, 1H), 7.91 (s, 1H). LCMS(ESI): [M+H]+ m/z: calcd 284.14; found 284.2.

### Step 5: The synthesis of 2-chloro-N-[[3-ethyl-4-[-methyl-4-(trifluoromethyl)imidazol-2-yl]phenyl]methyl]-5-nitro-pyrimidin-4-amine

DIPEA (1.15 g, 8.90 mmol, 1.55 mL) was added to a solution of [3-ethyl-4-[1-methyl-4-(trifluoromethyl)imidazol-2-yl]phenyl]methanamine (2.10 g, crude) and 2,4-dichloro-5-nitro-pyrimidine (1.44 g, 7.41 mmol) in ACN (100 mL). The reaction mixture was stirred at room temperature for 24 hr. The reaction mixture was concentrated under reduced pressure. The residue was diluted with EtOAc (300 mL) and washed with water (50 mL). The organic layer was separated, washed with water (20 mL) and brine (2×50 mL), dried over anhydrous sodium sulfate and concentrated under reduced pressure to afford 2-chloro-N-[[3-ethyl-4-[1-methyl-4-(trifluoromethyl)imidazol-2-yl]phenyl]methyl]-5-nitro-pyrimidin-4-amine (2.80 g, 6.35 mmol, 85.7% yield) as a brown solid which was used in the next steps without further purification.

LCMS(ESI): [M+H]+ m/z: calcd 441.11; found 441.0.

### Step 6: The synthesis of 2-chloro-N4-[[3-ethyl-4-[1-methyl-4-(trifluoromethyl)imidazol-2-yl]phenyl]methyl]pyrimidine-4,5-diamine

Zinc powder (2.49 g, 38.1 mmol) was added to a solution of 2-chloro-N-[[3-ethyl-4-[1-methyl-4-(trifluoromethyl)imidazol-2-yl]phenyl]methyl]-5-nitro-pyrimidin-4-amine (2.80 g, 6.35 mmol) and ammonium chloride (5.10 g, 95.3 mmo) in MeOH (200 mL) under argon atmosphere at 0° C. The reaction mixture was stirred at room temperature for 16 hr, then solids were filtered out. The filtrate was concentrated under reduced pressure. The residue was diluted with DCM (400 mL) and washed with water (100 mL). The organic layer was separated, washed with brine (100 mL) and concentrated under reduced pressure to afford 2-chloro-N4-[[3-ethyl-4-[1-methyl-4-(trifluoromethyl)imidazol-2-yl]phenyl]methyl]pyrimidine-4,5-diamine (2.30 g, 5.60 mmol, 88.1% yield) as a brown solid which was used in the next steps without further purification.

¹H NMR (500 MHz, DMSO-d6) δ 0.98 (t, 3H), 2.43-2.50 (m, 2H), 3.46 (s, 3H), 4.61 (d, 2H), 4.96 (s, 2H), 7.24 (d, 1H), 7.32 (d, 1H), 7.37 (s, 1H), 7.40-7.44 (m, 2H), 7.91 (s, 1H).

LCMS(ESI): [M+H]+ m/z: calcd 411.13; found 411.0.

### Step 7: The synthesis of 2-chloro-9-[[3-ethyl-4-[1-methyl-4-(trifluoromethyl)imidazol-2-yl]phenyl]methyl]-7H-purin-8-imine

BrCN (1.78 g, 16.8 mmol) was added to a solution of 2-chloro-N4-[[3-ethyl-4-[1-methyl-4-(trifluoromethyl)imidazol-2-yl]phenyl]methyl]pyrimidine-4,5-diamine (2.30 g, 5.60 mmol) in MeOH (100 mL). The reaction mixture was stirred at 40° C. for 72 hr. The reaction mixture was cooled to room temperature and concentrated under reduced pressure. The residue was diluted with MTBE (300 mL). The solid precipitate formed was filtered off and partitioned between EtOAc (200 mL) and saturated aqueous NaHCO3 solution (50 mL). The organic layer was separated, washed with brine (30 mL), dried over anhydrous sodium sulfate and concentrated under reduced pressure. The residue was subjected to flash column chromatography (SiO2, gradient elution: chloroform—methanol) to afford 2-chloro-9-[[3-ethyl-4-[1-methyl-4-(trifluoromethyl)imidazol-2-yl]phenyl]methyl]-7H-purin-8-imine (800 mg, 1.84 mmol, 32.8% yield) as a light-yellow solid.

¹H NMR (500 MHz, DMSO-d6) δ 0.98 (t, 3H), 2.43-2.50 (m, 2H), 3.46 (s, 3H), 5.34 (s, 2H), 7.06 (d, 1H), 7.32-7.38 (m, 2H), 7.50-7.55 (m, 2H), 7.92 (s, 1H), 8.31 (s, 1H).

LCMS(ESI): [M+H]+ m/z: calcd 436.13; found 436.0.

### Step 8: The synthesis of 2-chloro-9-[[3-ethyl-4-[1-methyl-4-(trifluoromethyl)imidazol-2-yl]phenyl]methyl]-7-(2,2,2-trifluoroethyl)purin-8-imine

Cesium carbonate (336 mg, 1.03 mmol) was added to a solution of 2-chloro-9-[[3-ethyl-4-[1-methyl-4-(trifluoromethyl)imidazol-2-yl]phenyl]methyl]-7H-purin-8-imine (300 mg, 688 mol) in ACN (8.0 mL). The reaction mixture was stirred at room temperature for 15 min. 2,2,2-trifluoroethyl trifluoromethanesulfonate (240 mg, 1.03 mmol, 149 μL) was added dropwise to the reaction mixture. The resulting mixture was stirred at 50° C. for 16 hr. The reaction mixture was cooled to room temperature, then solids were filtered out. The filtrate was concentrated under reduced pressure. The residue was diluted with EtOAc (30 mL) and washed with water (10 mL). The organic layer was separated, washed with brine (20 mL), dried over anhydrous sodium sulfate and concentrated under reduced pressure to afford 2-chloro-9-[[3-ethyl-4-[1-methyl-4-(trifluoromethyl)imidazol-2-yl]phenyl]methyl]-7-(2,2,2-trifluoroethyl)purin-8-imine (350 mg, crude) as a white solid which was used in the next step without further purification. LCMS(ESI): [M+H]+ m/z: calcd 518.13; found 518.

### Step 9: The synthesis of 2-[4-cyclopropyl-6-(trideuteriomethoxy)pyrimidin-5-yl]-9-[[3-ethyl-4-[1-methyl-4-(trifluoromethyl)imidazol-2-yl]phenyl]methyl]-7-(2,2,2-trifluoroethyl)purin-8-imine

The synthesis of 4-cyclopropyl-5-(4,4,5,5-tetramethyl-1,3,2-dioxaborolan-2-yl)-6-(trideuteriomethoxy)pyrimidine (I-56b) is described by Intermediate 56. [1310] 2-chloro-9-[[3-ethyl-4-[1-methyl-4-(trifluoromethyl)imidazol-2-yl]phenyl]methyl]-7-(2,2,2-trifluoroethyl)purin-8-imine (350 mg, 676 μmol), 4-cyclopropyl-5-(4,4,5,5-tetramethyl-1,3,2-dioxaborolan-2-yl)-6-(trideuteriomethoxy)pyrimidine (755 mg, 2.70 mmol), potassium phosphate tribasic anhydrous (430 mg, 2.03 mmol) and XPhosPdG3 (28.6 mg, 33.8 μmol) were mixed in a degassed mixture of dioxane (12 mL) and water (2 mL). The reaction mixture was stirred at 80° C. for 14 hr. under argon atmosphere. The reaction mixture was cooled to room temperature, diluted with EtOAc (25 mL) and washed with water (10 mL) and brine (2×20 mL) and dried over anhydrous sodium sulfate. SiliaMetS® Dimercaptotriazine (100 mg) was added to the resulting organic layer. The resulting mixture was stirred for 30 min and filtered. The filtrate was concentrated under reduced pressure. The residue was subjected to HPLC (0-2-10 min., 28-35-45% water—ACN, +0.1% vol. of 25% aq. NH3, flow: 30 mL/min, column: XBridge BEH C18 100×19 mm, 5 μm), then repurified by HPLC (2-10 min, 3-10-90% water+FA (0.1% vol.) -ACN+FA (0.1% vol.); flow: 30 mL/min, column: Chromatorex, l0×19 mm, 5 μm) to afford 2-[4-cyclopropyl-6-(trideuteriomethoxy)pyrimidin-5-yl]-9-[[3-ethyl-4-[1-methyl-4-(trifluoromethyl)imidazol-2-yl]phenyl]methyl]-7-(2,2,2-trifluoroethyl)purin-8-imine (51.0 mg, 80.4 μmol, 11.9% yield) as a yellow solid.

¹H NMR (600 MHz, DMSO-d6) δ 0.79-0.83 (m, 2H), 0.91 (t, 3H), 0.97-1.01 (m, 2H), 1.62-1.67 (m, 1H), 2.41 (q, 2H), 3.43 (s, 3H), 4.75-5.25 (m, 4H), 6.98-7.15 (m, 1H), 7.25 (d, 1H), 7.31 (d, 1H), 7.38 (s, 1H), 7.90 (s, 1H), 8.33 (br., 1H), 8.62 (s, 1H).

LCMS(ESI): [M+H]+ m/z: calcd 635.25; found 635.2.

### Example T-128

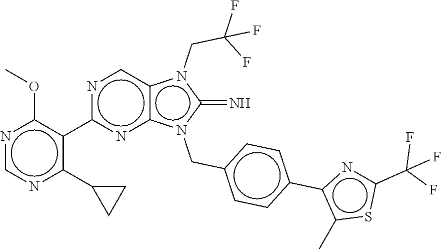

T-128 was made using a similar method as described for Example T-095, except where 4-[1-methyl-4-(trifluoromethyl)imidazol-2-yl]phenyl]methanamine is replaced with (4-(5-methyl-2-(trifluoromethyl)thiazol-4-yl)phenyl)methanamine.

¹H NMR (600 MHz, DMSO-d6) δ 0.78-0.85 (m, 2H), 0.97-1.10 (m, 2H), 1.65-1.70 (m, 1H), 2.61 (s, 3H), 3.83 (s, 3H), 4.78-4.85 (m, 1H), 4.97-5.04 (m, 1H), 5.10 (br. s, 1H), 5.23 (br. s, 1H), 7.04-7.13 (m, 1H), 7.45-7.52 (m, 2H), 7.62-7.66 (m, 2H), 8.27-8.37 (m, 1H), 8.63 (s, 1H).

LCMS(ESI): [M+H]+ m/z: calcd 621.2; found 621.2.

The synthesis of 4-(5-methyl-2-(trifluoromethyl)thiazol-4-yl)phenyl)methanamine

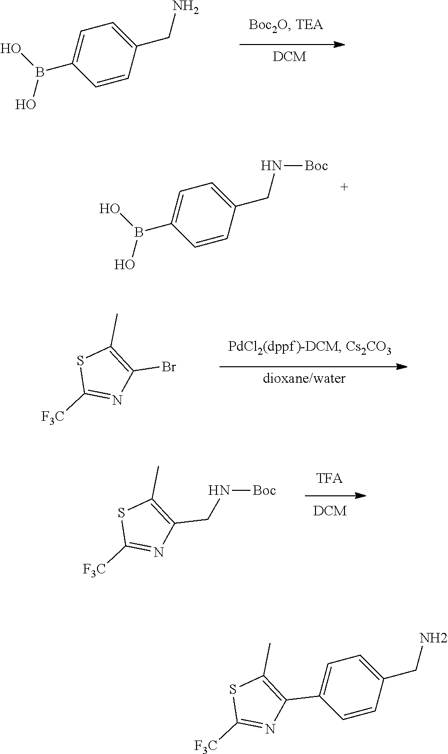 [SMILES: NCc1ccc(B(O)O)cc1 ; CC(C)(C)OC(=O)NCc1ccc(B(O)O)cc1 ; Cc1sc(C(F)(F)F)nc1Br ; Cc1sc(C(F)(F)F)nc1CNC(=O)OC(C)(C)C ; Cc1sc(C(F)(F)F)nc1-c1ccc(CN)cc1]

### Step 1: Synthesis of [4-[(tert-butoxycarbonylamino)methyl]phenyl]boronic acid

To a stirred solution of [4-(aminomethyl)phenyl]boronic acid HCl salt (3 g, 16.01 mmol, HCl) in DCM (1 mL) at 0° C. was added TEA (4.86 g, 48 mmol) followed by a solution tert-butoxycarbonyl tert-butyl carbonate (4.02 g, 18.4 mmol, 4.2 mL) over the period of 10 min. The resulting reaction mixture was stirred for 3 h at 0° C. then at room temperature for 16 hrs. The solution was treated with saturated aqueous citric acid solution (25 mL). The organic layer was separated, washed with brine (50 mL), water (50 mL), brine (50 mL), dried over MgSO4 and concentrated to give the title compound (3.7 g, 92% yield) which was used in a next step without further purifications.

### Step 2: Synthesis of tert-butyl N-[[4-[5-methyl-2-(trifluoromethyl)thiazol-4-yl]phenyl]methyl]carbamate

To a mixture of [4-[(tert-butoxycarbonylamino)methyl]phenyl]boronic acid (3.7 g, 11.79 mmol) and 4-bromo-5-methyl-2-(trifluoromethyl)thiazole (3.48 g, 14.15 mmol) in a mixture of dioxane/water (90 mL, 8:1) was added [1,1′-Bis(diphenylphosphino)ferrocene]palladium(II) dichloride (481 mg, 589 μmol) and cesium carbonate (11.52 g, 35.37 mmol) under a nitrogen atmosphere. After stirring at 85° C. for 14 hr, the reaction mixture was cooled to room temperature and concentrated under reduced pressure. The resulting residue was diluted with water (30 mL) and EtOAc (100 mL). The phases were separated and the organic phase was washed by water (2×30 mL), filtered through a diatomaceous earth and concentrated under reduced pressure to obtain crude title compound (3.85 g, 88% yield) which was used without further purification.

¹H NMR (500 MHz, CDCl3) δ 1.46 (s, 9H), 2.62 (s, 3H), 4.35 (br. s, 2H), 4.94 (br. s, 1H), 7.37 (d, 2H), 7.60 (d, 2H).

### Step 3: Synthesis of 4-(5-methyl-2-(trifluoromethyl)thiazol-4-yl)phenyl)methanamine

Tert-butyl N-[[4-[5-methyl-2-(trifluoromethyl)thiazol-4-yl]phenyl]methyl]carbamate (3.85 g, 8.79 mmol) was dissolved in DCM (50 mL). TFA (10 g, 87.9 mmol, 6.77 mL) was added dropwise to the resulting solution. The reaction mixture stirred at room temperature for 2 hrs. The reaction solution was made treated with a 30% aqueous potassium carbonate solution and the organic phase was separated. The organics were 30% aqueous potassium carbonate solution (2×15 mL). The organic layer was separated, dried over anhydrous sodium sulfate and evaporated in vacuum to dryness to obtain the title compound (2.15 g, 90% yield) which was used in the next without further purifications.

¹H NMR (500 MHz, CDCl₃) δ 2.63 (s, 3H), 3.85-3.96 (m, 2H), 7.41-7.73 (m, 6H).

### Example T-143

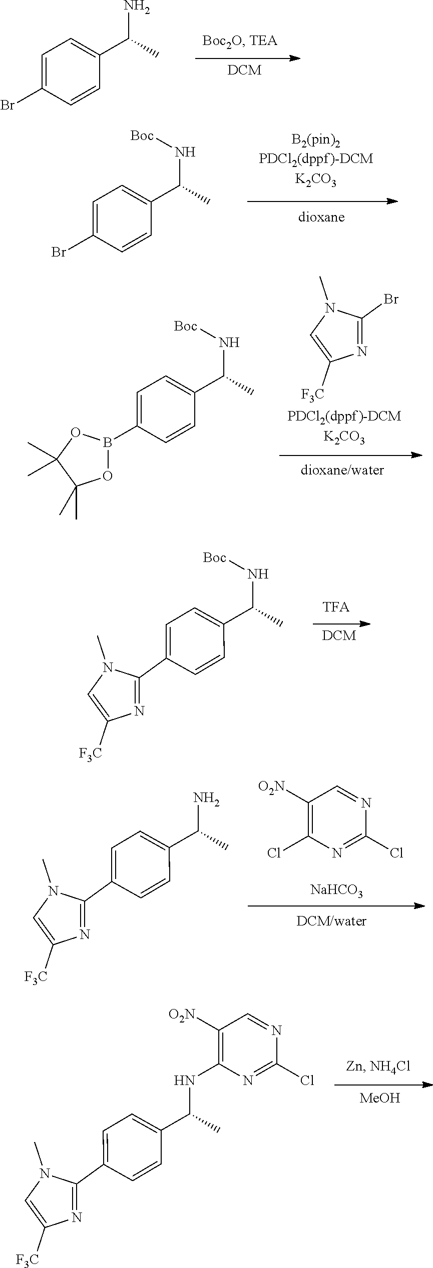

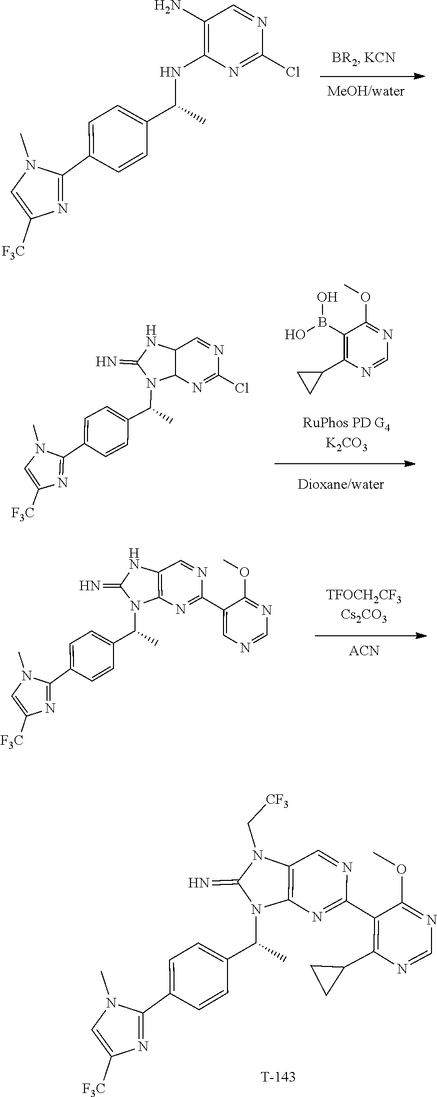

### Step 1: Synthesis of Tert-butyl (R)-(1-(4-bromophenyl)ethyl)carbamate

Triethylamine (7.59 g, 7.9 mmol, 10.45 mL) was added to solution of (R)-1-(4-bromophenyl)ethan-1-amine (10.0 g, 49.98 mmol) in DCM (186 mL). The mixture was cooled on an ice bath and tert-butoxycarbonyl tert-butyl carbonate (13.09 g, 59.98 mmol) was added thereto under an argon stream with stirring. The reaction mixture was stirred at 25° C. for 24 hr. The reaction solution was poured into water (200 mL) and the mixture was extracted with DCM (200 mL). The organic layer was successively washed with water, brine, dried over anhydrous sodium sulfate, and evaporated under reduced pressure to give the title compound (13 g, 87%) as a solid.

### Step 2: Synthesis of tert-butyl (R)-(1-(4-(4,4,5,5-tetramethyl-1,3,2-dioxaborolan-2-yl)phenyl)ethyl)carbamate

Tert-butyl (R)-(1-(4-bromophenyl)ethyl)carbamate (5 g, 16.7 mmol) was added to a solution of bis(pinacolato)diboron (4.65 g, 18.32 mmol), potassium acetate (3.27 g, 33 mmol) and [1,1′-Bis(diphenylphosphino)ferrocene]dichloropalladium(II), complex with dichloromethane (1.36 g, 1.67 mmol) in dioxane (200 mL). The reaction vessel was then evacuated and backfilled with argon three times. The mixture was stirred at 80° C. for 48 hr, then the mixture was cooled, filtered, and concentrated under reduced pressure to give the crude title compound (8 g), which was used in the next step without further purification.

LCMS(ESI): [M+H]⁺ m/z: calcd 348.3; found 371.0 [M+Na]⁺.

### Step 3: Synthesis of tert-butyl (R)-(1-(4-(1-methyl-4-(trifluoromethyl)-1H-imidazol-2-yl)phenyl)ethyl)carbamate

Dioxane (100 mL) and water (5 mL) were degassed 3×, followed by 2-bromo-1-methyl-4-(trifluoromethyl)imidazole (5.28 g, 23.04 mmol), tert-butyl (R)-(1-(4-(4,4,5,5-tetramethyl-1,3,2-dioxaborolan-2-yl)phenyl)ethyl)carbamate (8.00 g, 23 mmol), [1,1′-Bis(diphenylphosphino)ferrocene]dichloropalladium(II), complex with dichloromethane (1.88 g, 2.30 mmol) and potassium carbonate (6.37 g, 46 mmol) were added under argon atmosphere at 25° C. The reaction mixture was stirred for 12 hr at 100° C. The reaction mixture was cooled down, filtered and concentrated in vacuo. The crude product was subject to flash chromatography (gradient elution: hexane/ethyl acetate) to afford the title compound (2.5 g, 29% yield) as a solid.

LCMS(ESI): [M+H]⁺ m/z: calcd 300.1; found 247 [M-tBu].

### Step 4: Synthesis of (R)-1-(4-(1-methyl-4-(trifluoromethyl)-1H-imidazol-2-yl)phenyl)ethan-1-amine

To a stirred solution of tert-butyl (R)-(1-(4-(1-methyl-4-(trifluoromethyl)-1H-imidazol-2-yl)phenyl)ethyl)carbamate (2.5 g, 6.77 mmol) in DCM (30 mL) was trifluoroacetic acid (7.72 g, 67.7 mmol, 5.2 mL) and the reaction mixture was stirred for 12 hr at 25° C. The reaction mixture concentrated under reduced pressure and treated with 1 M NaOH (aq, 30 mL), then extracted with EtOAc (2×100 mL). The organic layer was washed with water (2×50 mL), dried over Na₂SO₄ and evaporated under reduced pressure to the crude title compound (2.1 g), which was taken forward without further manipulation.

LCMS(ESI): [M+H]⁺ m/z: calcd 270.1; found 270.2.

### Step 5: Synthesis of (R)-2-chloro-N-(1-(4-(1-methyl-4-(trifluoromethyl)-1H-imidazol-2-yl)phenyl)ethyl)-5-nitropyrimidin-4-amine

To a stirred solution of (R)-1-(4-(1-methyl-4-(trifluoromethyl)-1H-imidazol-2-yl)phenyl)ethan-1-amine TFA salt (2.1 g, 5.48 mmol) in DCM (20 mL) and sodium bicarbonate (3.28 g, 39 mmol) in water (10 mL) was added a solution of 2,4-dichloro-5-nitro-pyrimidine (6.31 g, 32.54 mmol) in DCM (20 mL) dropwise over 10 min at 0° C. The resulting reaction mixture was allowed to stir at 0° C. for 5.5 hr. The reaction mixture was diluted with water (50 mL) and the aqueous phase was extracted with DCM (100 mL). The combined organic phase was washed with water (50 mL), dried over Na2SO4 and concentrated under reduced pressure to give rise to the title compound (2.45 g, 18% yield).

LCMS(ESI): [M+H]⁺ m/z: calcd 427.1; found 427.0.

### Step 6: Synthesis of (R)-2-chloro-N4-(1-(4-(1-methyl-4-(trifluoromethyl)-1H-imidazol-2-yl)phenyl)ethyl)pyrimidine-4,5-diamine

To a solution of (R)-2-chloro-N-(1-(4-(1-methyl-4-(trifluoromethyl)-1H-imidazol-2-yl)phenyl)ethyl)-5-nitropyrimidin-4-amine (2.45 g, 5.74 mmol) and ammonium chloride (4.30 g, 80.4 mmol) in MeOH (50 mL) was added Zinc (2.25 g, 34 mmol). The reaction mixture was stirred at 0° C. for 1 hr, then filtered. The filtrate was evaporated in vacuo, and residue was dissolved in EtOAc (50 mL), washed with water (2×50 mL) dried over Na₂SO₄ and concentrated under reduced pressure to obtain the title compound (2.05 g, 90% yield).

LCMS(ESI): [M+H]⁺ m/z: calcd 397.1; found 397.0.

### Step 7: Synthesis of (R)-2-chloro-9-(1-(4-(1-methyl-4-(trifluoromethyl)-1H-imidazol-2-yl)phenyl)ethyl)-7,9-dihydro-8H-purin-8-imine

A solution of potassium cyanide (1.23 g, 18.90 mmol) in water (20 mL) as added slowly to a cooled at 0° C. stirred mixture of bromine (3.02 g, 18.90 mmol) in MeOH (50 mL). The resulting mixture was stirred at 0° C. for 15 min. To the above mixture, a solution of (R)-2-chloro-N4-(1-(4-(1-methyl-4-(trifluoromethyl)-1H-imidazol-2-yl)phenyl)ethyl)pyrimidine-4,5-diamine (2.5 g, 6.30 mmol) in water (10 mL) was slowly added at 0° C. The reaction mixture was stirred at 50° C. for 24 hr. The reaction mixture evaporated in vacuo, residue dissolved in MeOH (30 mL), filtered, and the filtrate concentrated in vacuo. The crude product was subject to flash chromatography to afford the title compound (1 g, 38% yield).

LCMS(ESI): [M+H]⁺ m/z: calcd 422.1; found 422.0.

### Step 8: Synthesis of (R)-2-(4-cyclopropyl-6-methoxypyrimidin-5-yl)-9-(1-(4-(1-methyl-4-(trifluoromethyl)-1H-imidazol-2-yl)phenyl)ethyl)-7,9-dihydro-8H-purin-8-imine

Dioxane (20 mL) and water (0.5 mL) were degassed 3×, followed by the addition of (R)-2-chloro-9-(1-(4-(1-methyl-4-(trifluoromethyl)-1H-imidazol-2-yl)phenyl)ethyl)-7,9-dihydro-8H-purin-8-imine (0.5 g, 1.19 mmol), (4-cyclopropyl-6-methoxy-pyrimidin-5-yl)boronic acid (670 mg, 3.56 mmol), RuPhos Pd G4 (101 mg, 118 μmol) and potassium carbonate (328 mg, 2.37 mmol) were added under argon atmosphere at 25° C. The reaction mixture was stirred for 12 hr at 100° C. The reaction mixture was cooled down, filtered and concentrated in vacuo to afford the crude title compound (0.6 g) which was taken forward without further manipulation.

LCMS(ESI): [M+H]⁺ m/z: calcd 536.2; found 536.0.

### Step 9: Synthesis of (R)-2-(4-cyclopropyl-6-methoxypyrimidin-5-yl)-9-(1-(4-(1-methyl-4-(trifluoromethyl)-1H-imidazol-2-yl)phenyl)ethyl)-7-(2,2,2-trifluoroethyl)-7,9-dihydro-8H-purin-8-imine

The 2,2,2-trifluoroethyl trifluoromethanesulfonate (286 mg, 1.23 mmol, 178 μL) was added dropwise to the stirred suspension of (R)-2-(4-cyclopropyl-6-methoxypyrimidin-5-yl)-9-(1-(4-(1-methyl-4-(trifluoromethyl)-1H-imidazol-2-yl)phenyl)ethyl)-7,9-dihydro-8H-purin-8-imine (0.6 g, 1.12 mmol) and cesium carbonate (438 mg, 1.34 mmol) in MeCN (15 mL) and allowed to stirred at 90° C. for 12 hr. Then the reaction mixture was evaporated, diluted with water (30 mL) and extracted with EtOAc (40 mL). The organic layer was washed with brine (30 mL), dried over sodium sulfate and concentrated under reduced pressure. The residue was purified by reverse phase HPLC (gradient elution: 30-45% of 0.1% NH₄OH (aq) in ACN/water) to give the title compound (18.9 mg, 2.7% yield).

¹H NMR (600 MHz, DMSO-d6) δ 0.74-0.84 (m, 2H), 0.96-0.98 (m, 2H), 1.65-1.69 (m, 1H), 1.95 (d, 3H), 3.73 (s, 3H), 3.81 (s, 3H), 4.76-4.83 (m, 1H), 4.90-4.99 (m, 1H), 5.84-5.93 (m, 1H), 6.9-7.1 (m, 1H), 7.52 (d, 2H), 7.64 (d, 2H), 7.89 (s, 1H), 8.28-8.36 (m, 1H), 8.6 (s, 1H).

LCMS(ESI): [M+H]⁺ m/z: calcd 618.2; found 618.2.

### Example T-114

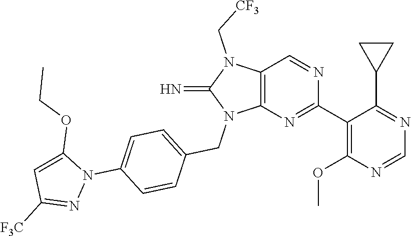 [SMILES: CCOc1cc(C(F)(F)F)nn1-c1ccc(Cn2c(=N)n(CC(F)(F)F)c3cnc(-c4c(OC)ncnc4C4CC4)nc32)cc1]

T-114 was made using a similar method as described for Example T-117, except where 4-(5-methoxy-3-(trifluoromethyl)-1H-pyrazol-1-yl)phenyl)methanamine is replaced with (4-(5-ethoxy-3-(trifluoromethyl)-1H-pyrazol-1-yl)phenyl)methanamine.

¹H NMR (600 MHz, DMSO-d6) δ 0.78-0.83 (m, 2H), 0.97-1.07 (m, 2H), 1.31 (t, 3H), 1.64-1.69 (m, 1H), 3.81 (s, 3H), 4.24 (q, 2H), 4.79 (br. s, 1H), 4.97 (br. s, 1H), 5.07 (br. s, 1H) 5.20 (br. s, 1H), 6.42 (s, 1H), 7.06-7.09 (m, 1H), 7.48 (d, 2H), 7.59 (d, 2H), 8.31-8.34 (m, 1H), 8.62 (s, 1H).

LCMS(ESI): [M+H]+ m/z: calcd 634.2; found 634.2.

### Example T-132

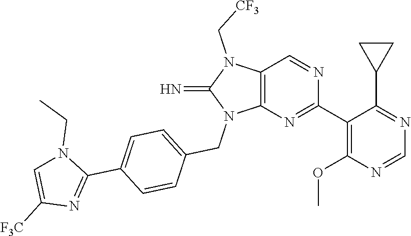 [SMILES: CCn1cc(C(F)(F)F)nc1-c1ccc(Cn2c(=N)n(CC(F)(F)F)c3cnc(-c4c(OC)ncnc4C4CC4)nc32)cc1]

T-132 was made using a similar method as described for Example T-095, except where 4-[1-methyl-4-(trifluoromethyl)imidazol-2-yl]phenyl]methanamine is replaced with (4-(1-ethyl-4-(trifluoromethyl)-1H-imidazol-2-yl)phenyl)methanamine.

¹H NMR (600 MHz, DMSO-d6) δ 0.78-0.85 (m, 2H), 0.96-1.15 (m, 2H), 1.32 (t, 3H), 1.65-1.70 (m, 1H), 3.82 (s, 3H), 4.05 (q, 2H), 4.80-5.05 (m, 2H), 5.12 (br. s, 1H), 5.24 (br. s, 1H), 7.13-7.23 (m, 1H), 7.47-7.50 (m, 2H), 7.55-7.61 (m, 2H), 8.02 (s, 1H), 8.31-8.35 (m, 1H), 8.61-8.65 (m, 1H).

LCMS(ESI): [M+H]+ m/z: calcd 618.2; found 618.2.

### Example T-101

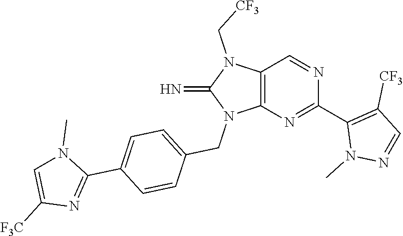 [SMILES: Cn1cc(C(F)(F)F)nc1-c1ccc(Cn2c(=N)n(CC(F)(F)F)c3cnc(-c4c(C(F)(F)F)cnn4C)nc32)cc1]

T-101 was made using a similar method as described for Example T-130, except where 1-isopropyl-4-methyl-5-(4,4,5,5-tetramethyl-1,3,2-dioxaborolan-2-yl)-1H-pyrazole is replaced with (1-methyl-5-(4,4,5,5-tetramethyl-1,3,2-dioxaborolan-2-yl)-4-(trifluoromethyl)-1H-pyrazole.

¹H NMR (600 MHz, DMSO-d6) δ 3.73 (s, 3H), 3.99 (s, 3H), 4.83 (br. s, 1H), 5.05 (br. s, 1H), 5.15 (br. s, 1H), 5.23 (br. s, 1H), 7.22-7.25 (m, 1H), 7.49 (d, 2H), 7.66 (d, 2H), 7.89 (s, 2H), 8.39 (d, 1H).

LCMS(ESI): [M+H]+ m/z: calcd 604.2; found 604.0.

The compounds in Table 2 were prepared according to one or more of the General Schemes.

TABLE 2
Additional compounds and experimental data

| Cmpd | | Relevant |
|---|---|---|
| No. | Structure | Scheme(s) |
| T-025 | 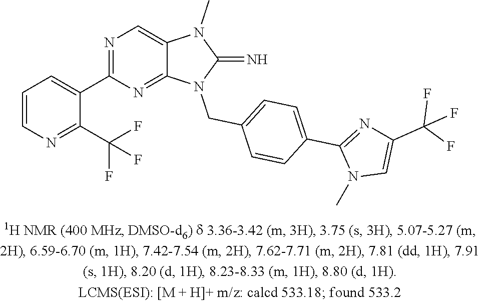 [SMILES: Cn1cc(C(F)(F)F)nc1-c1ccc(Cn2c(=N)n(C)c3cnc(-c4cccnc4C(F)(F)F)nc32)cc1] | 1, 15 |
| T-045 | 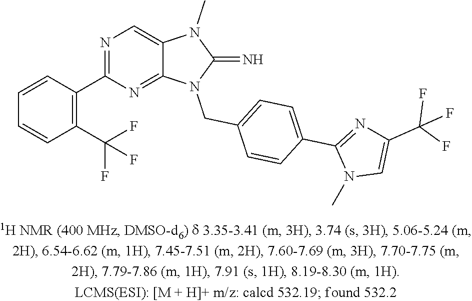 [SMILES: Cn1cc(C(F)(F)F)nc1-c1ccc(Cn2c(=N)n(C)c3cnc(-c4ccccc4C(F)(F)F)nc32)cc1] | 1, 15 |
| T-034 | 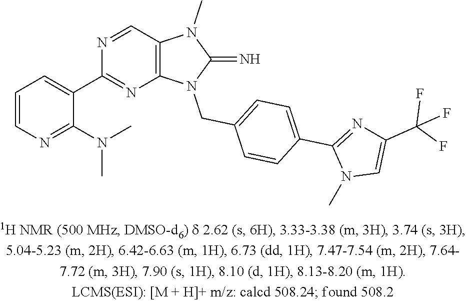 [SMILES: CN(C)c1ncccc1-c1ncc2c(n1)n(Cc1ccc(-c3nc(C(F)(F)F)cn3C)cc1)c(=N)n2C] | 1, 15 |
| T-090 | 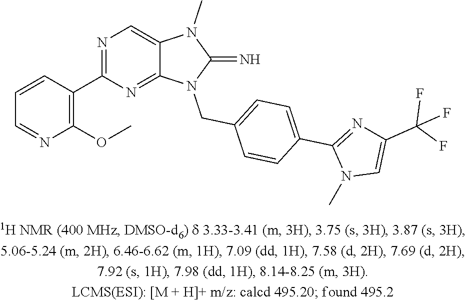 [SMILES: COc1ncccc1-c1ncc2c(n1)n(Cc1ccc(-c3nc(C(F)(F)F)cn3C)cc1)c(=N)n2C] | 1, 15 |
| T-074 | 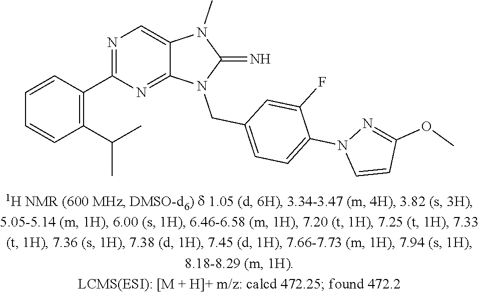 [SMILES: COc1ccn(-c2ccc(Cn3c(=N)n(C)c4cnc(-c5ccccc5C(C)C)nc43)cc2F)n1] | 1, 13, 15 |
| T-050 | 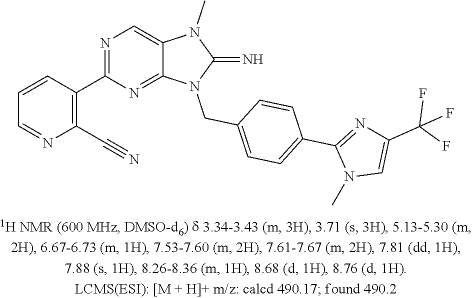 [SMILES: Cn1cc(C(F)(F)F)nc1-c1ccc(Cn2c(=N)n(C)c3cnc(-c4cccnc4C#N)nc32)cc1] | 1, 15 |
| T-075 | 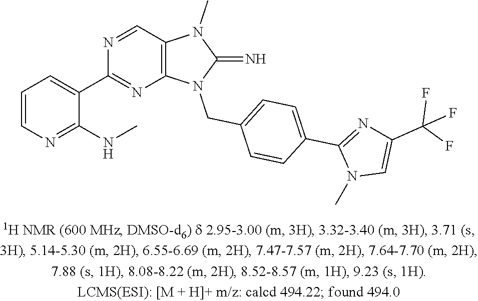 [SMILES: CNc1ncccc1-c1ncc2c(n1)n(Cc1ccc(-c3nc(C(F)(F)F)cn3C)cc1)c(=N)n2C] | 1, 14, 15 |
| T-041 | 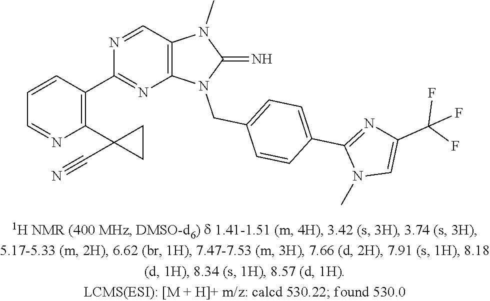 [SMILES: Cn1cc(C(F)(F)F)nc1-c1ccc(Cn2c(=N)n(C)c3cnc(-c4cccnc4C4(C#N)CC4)nc32)cc1] | 1, 15 |
| T-085 | 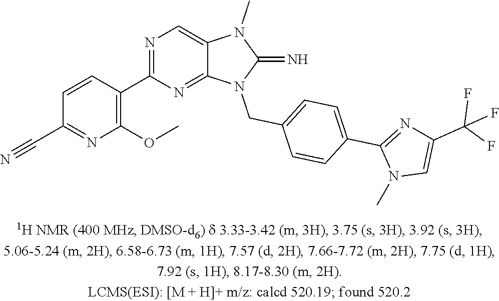 [SMILES: COc1nc(C#N)ccc1-c1ncc2c(n1)n(Cc1ccc(-c3nc(C(F)(F)F)cn3C)cc1)c(=N)n2C] | 1, 15 |
| T-023 | 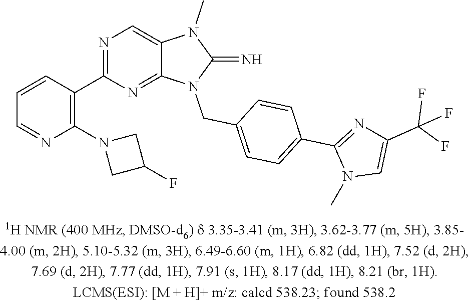 [SMILES: Cn1cc(C(F)(F)F)nc1-c1ccc(Cn2c(=N)n(C)c3cnc(-c4cccnc4N4CC(F)C4)nc32)cc1] | 1, 14, 15 |
| T-064 | 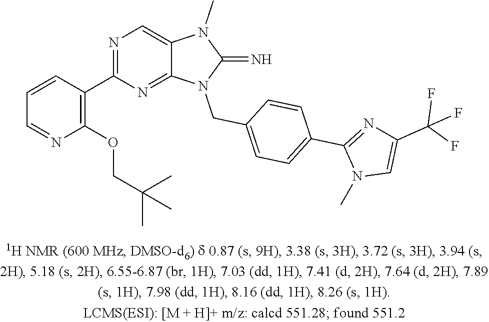 [SMILES: Cn1cc(C(F)(F)F)nc1-c1ccc(Cn2c(=N)n(C)c3cnc(-c4cccnc4OCC(C)(C)C)nc32)cc1] | 1, 14, 15 |
| T-020 | 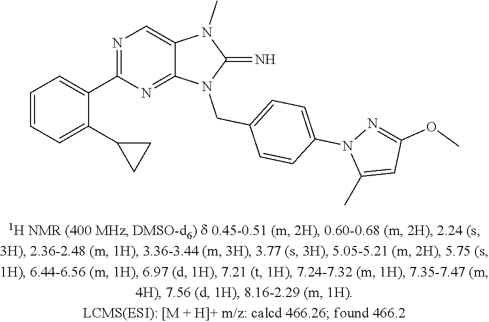 [SMILES: COc1cc(C)n(-c2ccc(Cn3c(=N)n(C)c4cnc(-c5ccccc5C5CC5)nc43)cc2)n1] | 1, 13, 15 |
| T-096 | 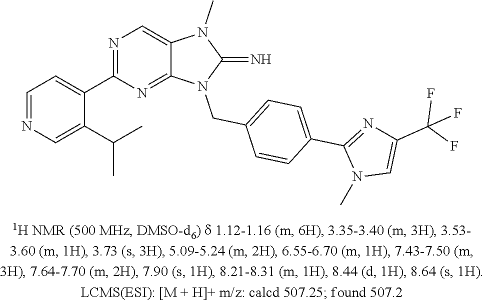 [SMILES: CC(C)c1cnccc1-c1ncc2c(n1)n(Cc1ccc(-c3nc(C(F)(F)F)cn3C)cc1)c(=N)n2C] | 1, 15 |
| T-035 | 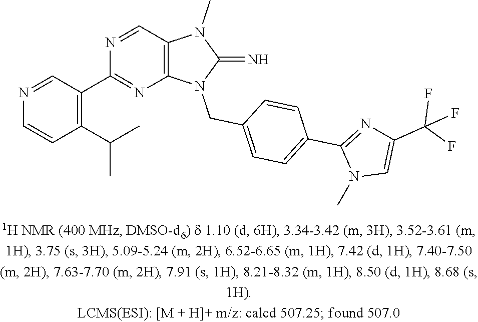 [SMILES: CC(C)c1ccncc1-c1ncc2c(n1)n(Cc1ccc(-c3nc(C(F)(F)F)cn3C)cc1)c(=N)n2C] | 1, 15 |
| T-013 | 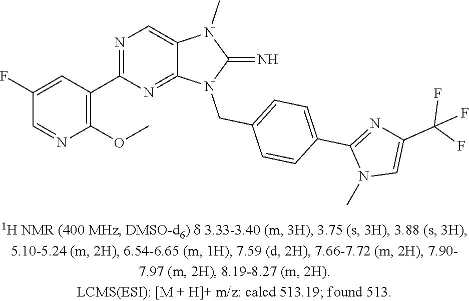 [SMILES: COc1ncc(F)cc1-c1ncc2c(n1)n(Cc1ccc(-c3nc(C(F)(F)F)cn3C)cc1)c(=N)n2C] | 1, 15 |
| T-037 | 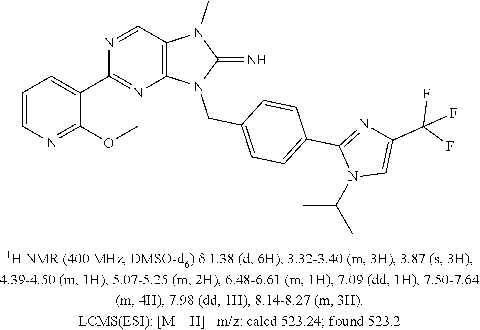 [SMILES: COc1ncccc1-c1ncc2c(n1)n(Cc1ccc(-c3nc(C(F)(F)F)cn3C(C)C)cc1)c(=N)n2C] | 1, 15 |
| T-031 | 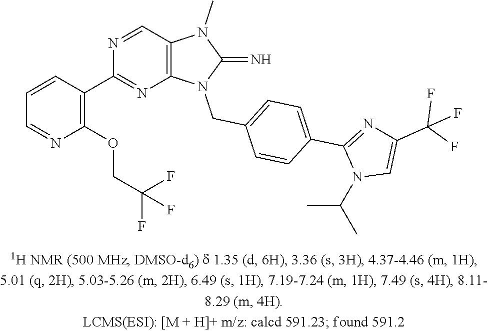 [SMILES: CC(C)n1cc(C(F)(F)F)nc1-c1ccc(Cn2c(=N)n(C)c3cnc(-c4cccnc4OCC(F)(F)F)nc32)cc1] | 1, 15 |
| T-057 | 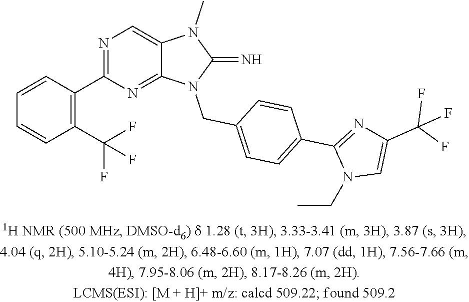 [SMILES: CCn1cc(C(F)(F)F)nc1-c1ccc(Cn2c(=N)n(C)c3cnc(-c4ccccc4C(F)(F)F)nc32)cc1] | 1, 15 |

### Biology Example 1. Measurement of Inhibition of Deubiquitinase Activity by Exemplary Compounds

Deubiquitinase activity of USP1-UAF1 was measured using Ubiquitin-rhodamine 110 as a substrate. Cleavage of amide bond between rhodamine and C-terminal Glycine of Ubiquitin peptide yields Rhodamine 1 10-Gly, leading to an increase of fluorescence signal. The assay buffer consisted of 50 mM HTEPES (pH 7.0), 100 DMSO, 0.0100 Bovine Serum Albumin, 1 mM TCEP, 0.00500 Tween-20. Total assay volume was 20 Li.

Compounds depicted below were dissolved in 10 mM DMSO stock and enzyme inhibition was measured in dose response format with top concentration of 10 μM in final assay well. 10 μL of enzyme buffer mix consisting of 1 nM USP1-UAF1 in the assay buffer describe above was added to compounds and incubated at ambient temperature for 30 min. 10 μL of substrate mix consisting of 200 nM Ubiquitin-Rho1 10 was added to initiate the deubiquitinate reaction catalyzed by USP 1/UAF 1. End point fluorescence intensity of USP1/UAF1 deubiquitinase product, Rhodamine 110-Gly, was measured at Excitiation of 480 nm/Emission at 540 nm.

Percentage of activity was calculated by normalization of fluorescence intensity to control wells using the following equations: % Activity=100*((FI_{observed}−Min)/(Max−Min)−1) where FI_{observed} is the fluorescence intensity read out of the compound of interest samples, Min and Max is the fluorescence intensity of control well samples consisting of 1 mM of known USP1-UAF1 inhibitor probe ML-323 and DMSO controls respectively. IC₅₀ values were calculated using the standard dose response fit in Genedata Screener® where the top and bottom were fixed to 0 and -100 respectively.

### Biology Example 2. Cellular Viability Assay

For short-term viability assays, cells were seeded in triplicates in 384-well plates one day prior to compound addition. Cells were incubated for 10 days with DMSO and increasing concentration of compounds. Cell viability was determined at end of the assay using Cell Titer-Glo Luminescence Assay (Promega) following manufacturer's instructions using an EnVision plate reader (Perkin Elmer). Measured values were normalized using DMSO control wells (100%) and complete cell kill control (0%; 10 μM MG132).

For long-term colony formation assays, cells were seeded in 12-well or 6-well plates at a very low density one day prior to compound addition. Cells were incubated for 7-21 days, depending on the cell line doubling time, with DMSO and increasing concentration of compounds. Media containing fresh compound was replenished every 3-4 days. At end of the incubation period, cells were stained with 0.1% crystal violent in 10% methanol for 10 minutes at room temperature. Stained plates were scanned and quantified using the Li-Cor Odyssey imaging system.

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment disclosed herein that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope disclosed herein, as defined in the following claims.

1. A compound of Formula (I) or a pharmaceutically acceptable salt, hydrate, solvate, prodrug, stereoisomer, or tautomer thereof;
wherein: 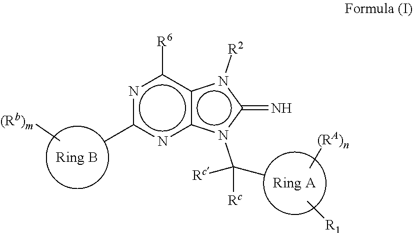
Ring B is a 5-6 member monocyclic aryl or heteroaryl;
Ring A is selected from C₆-C₁₀ aryl, 5-10 membered heteroaryl, —C₃-C₁₀ cycloalkyl, and 3-10 membered heterocyclyl;
R¹ is an optionally substituted 5-10 membered heteroaryl or an optionally substituted 3-10 membered heterocyclyl;
R² is selected from H, —C₁-C₆ alkyl, —C₁-C₆ haloalkyl, —C₁-C₆ heteroalkyl, —C₁-C₆ hydroxyalkyl, —C₃-C₁₀ cycloalkyl and arylalkyl, wherein each hydrogen of the alkyl, haloalkyl, heteroalkyl, hydroxyalkyl and arylalkyl can be independently replaced with a deuterium atom;
R⁶ is selected from H, -D, halo, —CN, —C₁-C₆ alkyl, —C₁-C₆ alkynyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, 6-10 member heteroaryl, heterocyclylalkyl, heteroarylalkyl, arylalkyl, cycloalkylalkyl, —OR^{a6}, —N(R^{a6})₂, —C(═O)R^{a6}, —C(═O)OR^{a6}, —NR^{a6}C(═O)R^{a6}, —NR^{a6}C(═O)OR^{a6}, —C(═O)N(R^{a6})₂, and —OC(═O)N(R^{a6})₂, wherein each alkyl, alkynyl, cycloalkyl, heterocyclyl, aryl, heteroaryl, arylalkyl and heteroarylalkyl is optionally substituted at any available position;
each R^{a6} is independently selected from H, —C₁-C₆ alkyl, —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl;
each R^{A} is independently selected from -D, halo, —CN, —C₁-C₆ alkyl, —C₁-C₆ haloalkyl, —C₁-C₆ hydroxyalkyl, —C₃-C₁₀ cycloalkyl, —OR^{A1}, —N(R^{A1})₂;
each R^{A1} is independently selected from H, —C₁-C₆ alkyl, —C₁-C₆ haloalkyl and C₃-C₉ cycloalkyl;
each R^{b} is independently selected from D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{b}, —N(R^{b1})₂, —C(═O)R^{b1}, —C(═O)OR^{b1}, —NR^{b1}C(O)R^{b1}, —NR^{b1}C(O)OR^{b1}, —C(═O)N(R^{b1})₂, —OC(═O)N(R^{b1})₂, —S(═O)R^{b1}, —S(═O)₂R^{b1}, —SR^{b1}, —S(═O)(═NR^{b1})R^{b1}, —R^{b1}S(═O)₂R^{b1} and —S(═O)₂N(R^{b1})₂ or 2 R^{b} together with the atoms to which they are attached form a 4-7 member carbocyclyl or a 4-7 member heterocyclyl, wherein each alkyl, carbocyclvl, cycloalkyl, heterocyclyl, aryl, heteroaryl, arylalkyl and heteroarylalkyl of R^{b} is optionally substituted at any available position;
each R^{b1} is independently selected from H, —C₁-C₆ alkyl (wherein each hydrogen can be independently replaced by deuterium), —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl;
each R^{c} and R^{c′} is independently selected from H, -D, —C₁-C₆ alkyl, —C₁-C₆ heteroalkyl and —C₁-C₆ haloalkyl or R^{c} and R^{c′} can be taken together with the atom to which they are attached to form a —C₃-C₉ cycloalkyl or a carbonyl;
n is 0, 1, 2 or 3; and
m is 0, 1, 2 or 3.

2-3. (canceled)

4. The compound of claim 1 or a pharmaceutically acceptable salt, hydrate, solvate, prodrug, stereoisomer, or tautomer thereof, wherein each R^{b} is independently selected from —CN, —C(═CH₂)CH₃, —F, -ⁱPr, —CF₃, cyclopropyl (substituted with 0, 1 or 2 instances of —F, -Me, —CN), —OCF₃, —OCHF₂, and —OMe.

5-6. (canceled)

7. The compound of claim 1 or a pharmaceutically acceptable salt, hydrate, solvate, prodrug, stereoisomer, or tautomer thereof, wherein the compound is of Formula (II)
wherein: 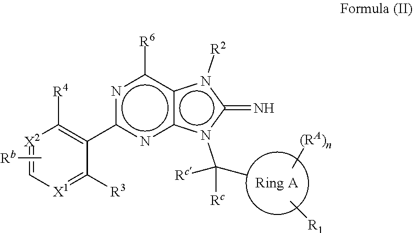
X¹ is selected from CH and N;
X² is selected from CH and N;
R³ is selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂ wherein each alkyl, cycloalkyl, heterocyclyl, aryl, heteroaryl, arylalkyl and heteroarylalkyl is optionally substituted at any available position;
R⁴ is selected from H, -D, halo, —CN, —C₁-C₆ alkyl, —C₁-C₆ alkenyl, —C₁-C₆ heteroalkyl, —C₁-C₆ haloalkyl, —C₁-C₆ hydroxyalkyl, —C₃-C₁₀ cycloalkyl, 3-10 membered heterocyclyl, —C₆-C₁₀ aryl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂ wherein each alkyl, cycloalkyl, heterocyclyl, aryl, heteroaryl, arylalkyl and heteroarylalkyl is optionally substituted at any available position; and
each R^{a3} and R^{a4} is independently selected from H, —C₁-C₆ alkyl (wherein each hydrogen can be replaced by deuterium), —C₁-C₆ heteroalkyl, —C₁-C₆ haloalkyl, —C₃-C₉ cycloalkyl, 3-7 membered heterocyclyl, cycloalkylalkyl, heterocyclylalkyl, aryl, 5-6 membered heteroaryl, arylalkyl and heteroarylalkyl.

8. The compound of claim 7 or a pharmaceutically acceptable salt, hydrate, solvate, prodrug, stereoisomer, or tautomer thereof, wherein the moiety represented by 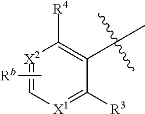
is selected from:

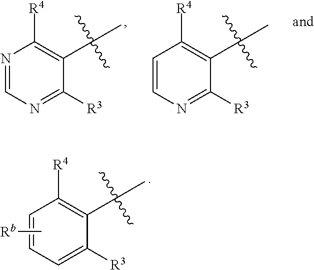

9. (canceled)

10. The compound of claim 7 or a pharmaceutically acceptable salt, hydrate, solvate, prodrug, stereoisomer, or tautomer thereof, wherein each R³ is independently selected from H, -D, —CN, —C(═CH₂)CH₃, —C(CH₃)CH₂CH₃, —Cl, —F, -Me, -ⁱPr, —CH₂N(CH₃)CH₂CF₃, —CF₃, —CH₂CF₃, cyclopropyl (substituted with 0 or 1 instance of —CN), azetidinyl (substituted with 0 or 1 instances of —F), phenyl (substituted with 0 or 1 instances of halo), —OCF₃, —OCH₂CF₃, —OCHF₂, —OCH₂F, —OⁱPr, —OMe, -OEt, -OCD₃, —OCH₂CH(CH₃)₃, —N(Me)₂, —NHMe and —NHⁱPr.

11. (canceled)

12. The compound of claim 7 or a pharmaceutically acceptable salt, hydrate, solvate, prodrug, stereoisomer, or tautomer thereof, wherein each R⁴ is independently selected from H, -D, —CN, —C(═CH₂)CH₃, —C(CH₃)CH₂CH₃, —Cl, —F, -Me, -ⁱPr, —CH₂N(CH₃)CH₂CF₃, —CF₃, —CH₂CF₃, cyclopropyl (substituted with 0, 1 or 2 instances of —CN, —F, or -Me), azetidinyl (substituted with 0 or 1 instances of —F), phenyl (substituted with 0 or 1 instances of halo), —OCF₃, —OCH₂CF₃, —OCHF₂, —OⁱPr, —OMe, —OCH₂CH(CH₃)₃, —N(Me)₂ and —NHMe and —NHⁱPr.

13. The compound of claim 7 or a pharmaceutically acceptable salt, hydrate, solvate, prodrug, stereoisomer, or tautomer thereof, wherein each R⁴ is selected from H and —OMe.

14. The compound claim 1 or a pharmaceutically acceptable salt, hydrate, solvate, prodrug, stereoisomer, or tautomer thereof, wherein R^{c} and R^{c′} are each independently selected from H and -Me or are taken together to form a cyclopropyl group.

15. The compound of claim 1 or a pharmaceutically acceptable salt, hydrate, solvate, prodrug, stereoisomer, or tautomer thereof, wherein the moiety represented by 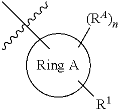
is selected from

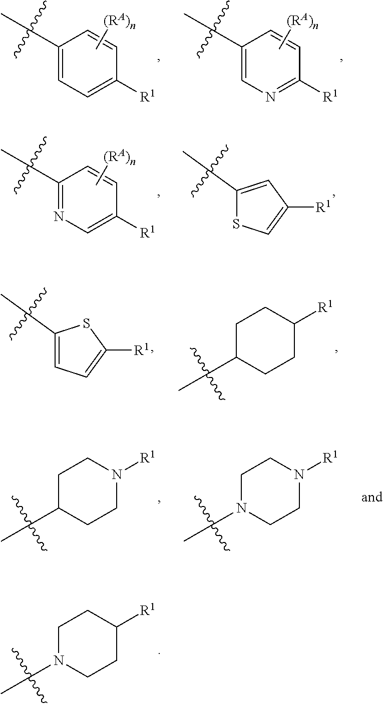

16. The compound of claim 1 or a pharmaceutically acceptable salt, hydrate, solvate, prodrug, stereoisomer, or tautomer thereof, wherein the moiety represented by

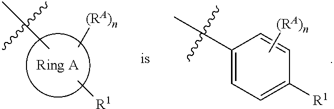

17. (canceled)

18. The compound of claim 1 or a pharmaceutically acceptable salt, hydrate, solvate, prodrug, stereoisomer, or tautomer thereof, wherein each R^{A} is independently selected from —F, —Cl, -Me, —OH and —OMe.

19-20. (canceled)

21. The compound of claim 1 or a pharmaceutically acceptable salt, hydrate, solvate, prodrug, stereoisomer, or tautomer thereof, wherein R¹ is imidazolyl or pyrazolyl, each substituted with 0, 1, 2 or 3 instances of R⁵.

22. The compound of claim 21 or a pharmaceutically acceptable salt, hydrate, solvate, prodrug, stereoisomer, or tautomer thereof, wherein R⁵ is selected from —CN, —F, —Cl, —Br, -Me, -Et, -ⁱPr, —CF₃, —CH₂CH₂F, —CH₂CHF₂, —OMe, -OEt, —CH₂CH₂OMe, —CH₂CH₂OH, cyclopropyl, oxetanyl and azetidinyl.

23. The compound of claim 1 or a pharmaceutically acceptable salt, hydrate, solvate, prodrug, stereoisomer, or tautomer thereof, wherein R¹ is selected from:

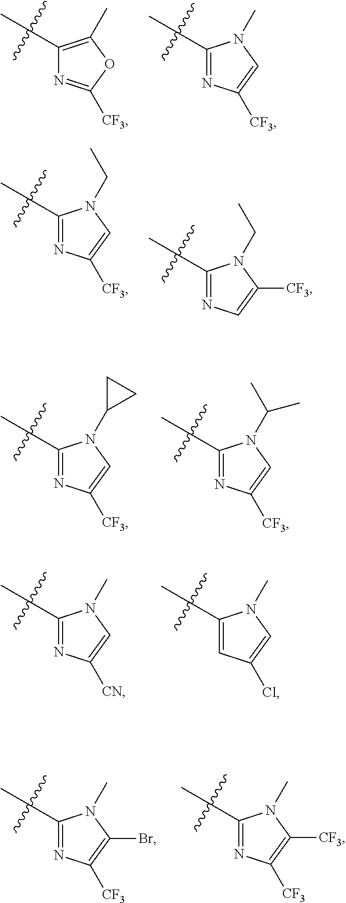

24. The compound of claim 1 or a pharmaceutically acceptable salt, hydrate, solvate, prodrug, stereoisomer, or tautomer thereof, wherein R¹ is selected from:

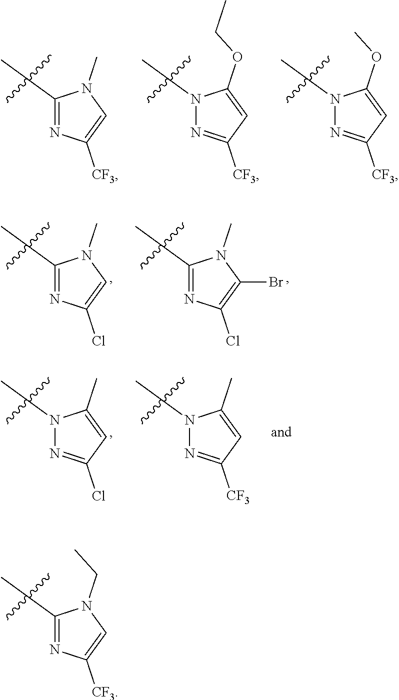

25. The compound of claim 1 or a pharmaceutically acceptable salt, hydrate, solvate, prodrug, stereoisomer, or tautomer thereof, wherein R² is selected from —C₁-C₆ alkyl, —C₁-C₆ haloalkyl, —C₁-C₆ heteroalkyl, —C₃-C₁₀ cycloalkyl wherein each hydrogen of the alkyl, haloalkyl and heteroalkyl can be independently replaced with a deuterium atom.

26. (canceled)

27. The compound of claim 1 or a pharmaceutically acceptable salt, hydrate, solvate, prodrug, stereoisomer, or tautomer thereof, wherein R² is selected from -Me, -Et, —CH₂CHF₂, —CH₂CF₃, cyclobutyl and —CH₂CH₂OMe.

28. (canceled)

29. The compound of claim 1 or a pharmaceutically acceptable salt, hydrate, solvate, prodrug, stereoisomer, or tautomer thereof, wherein R⁶ is H.

30. The compound of claim 1 or a pharmaceutically acceptable salt, hydrate, solvate, prodrug, stereoisomer, or tautomer thereof, wherein the compound is selected from

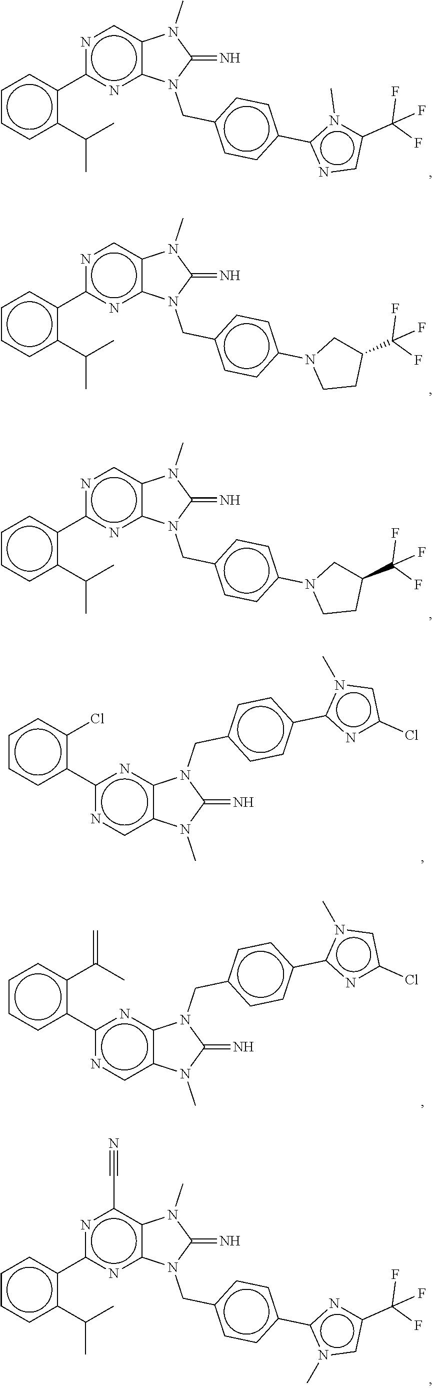

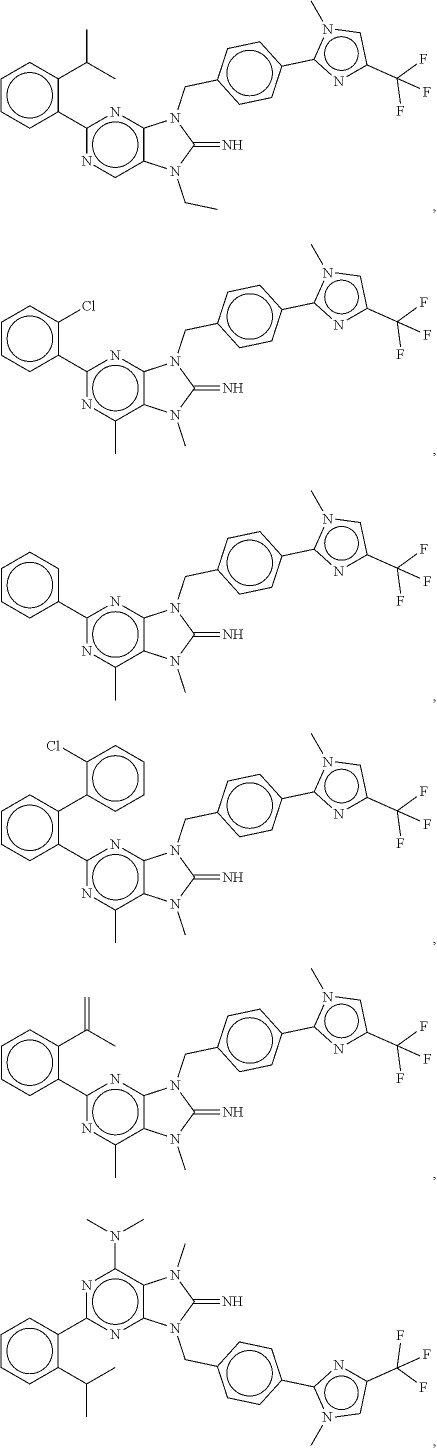

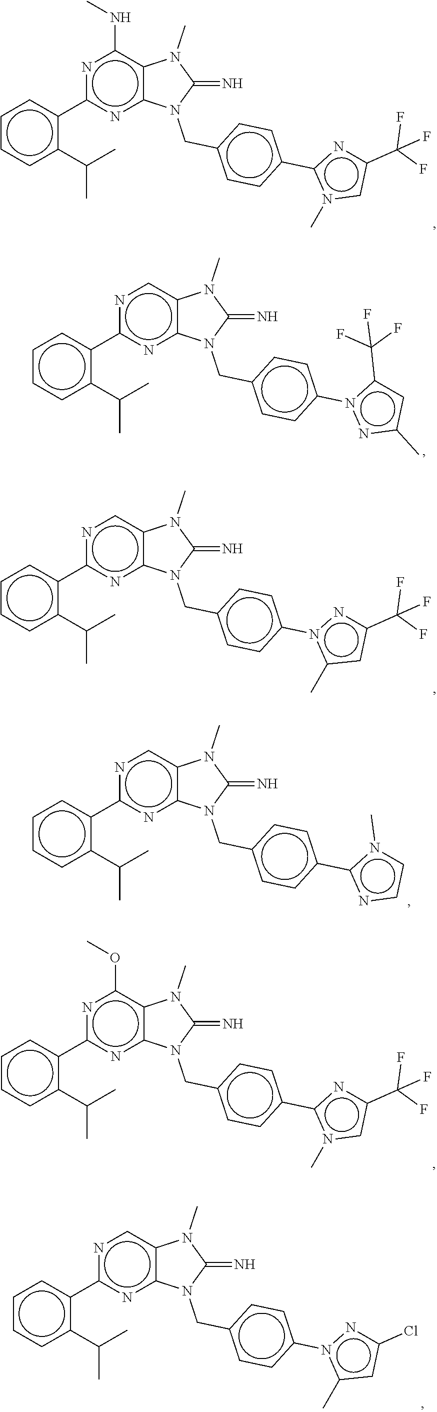

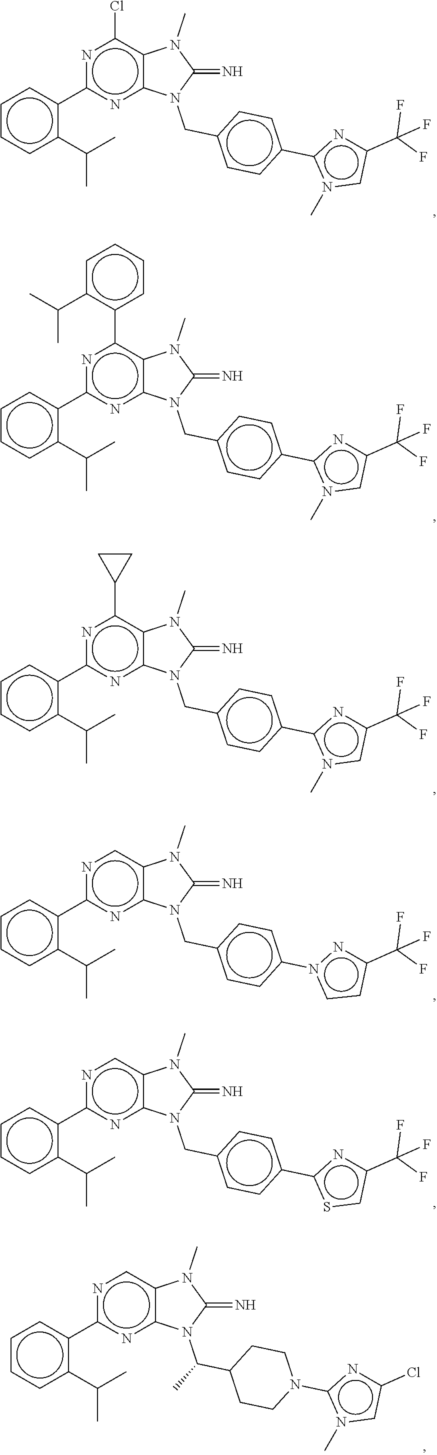

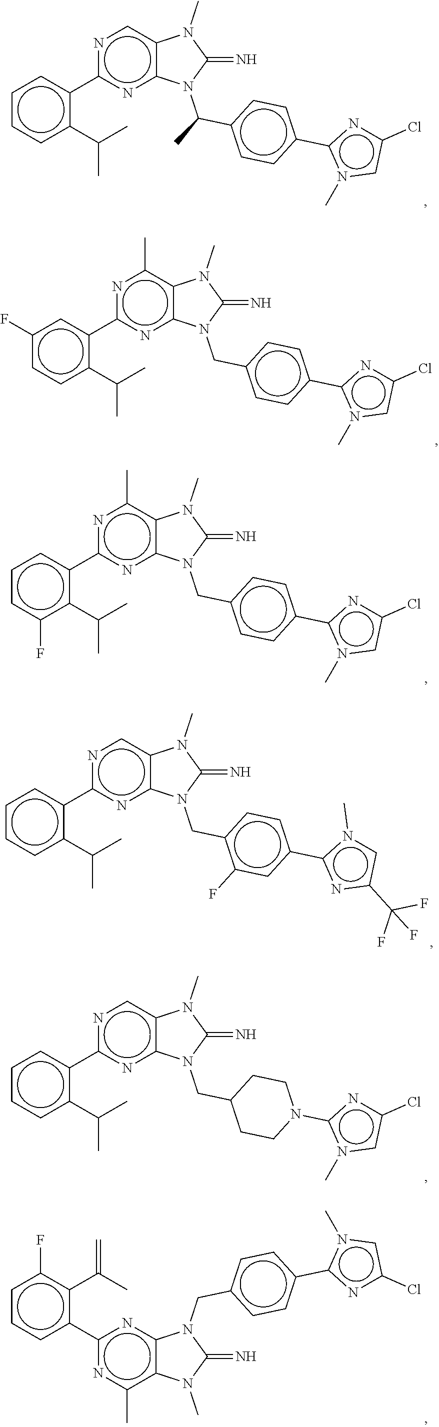

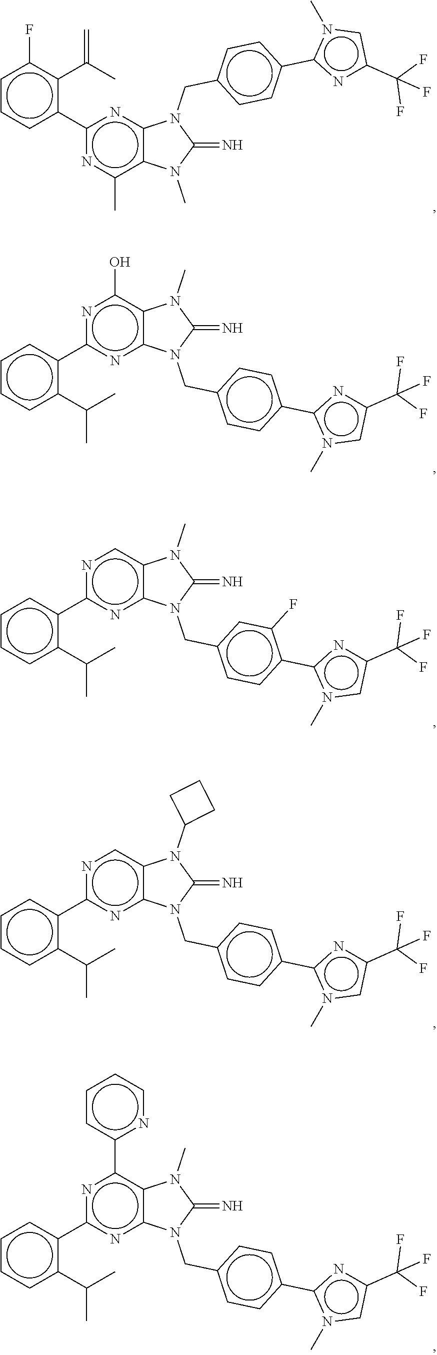

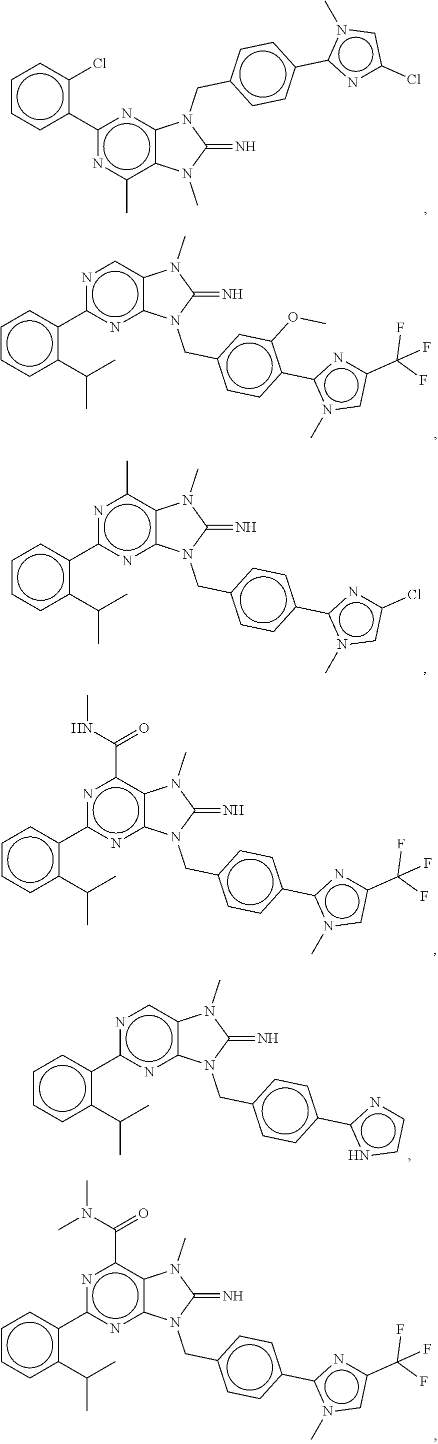

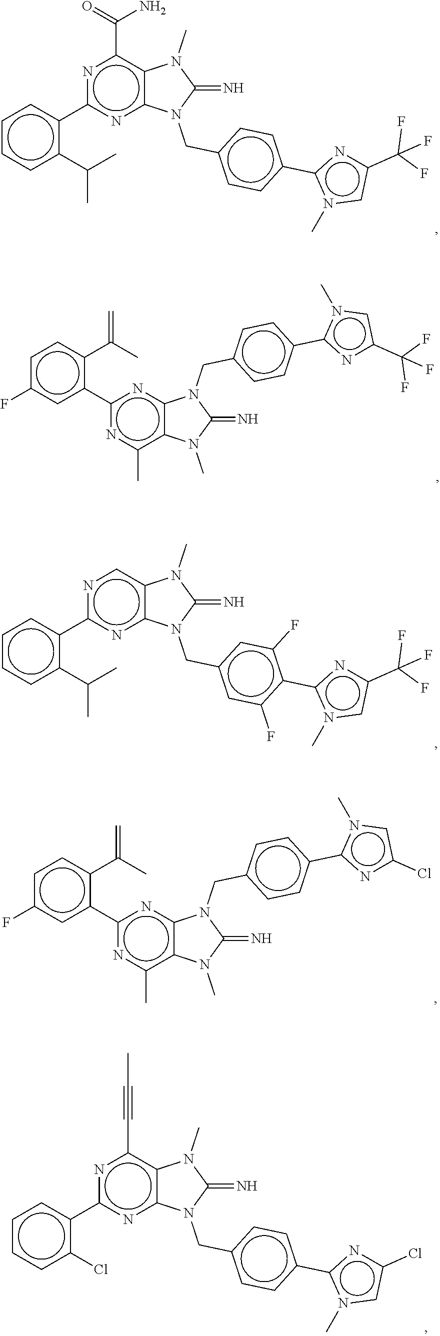

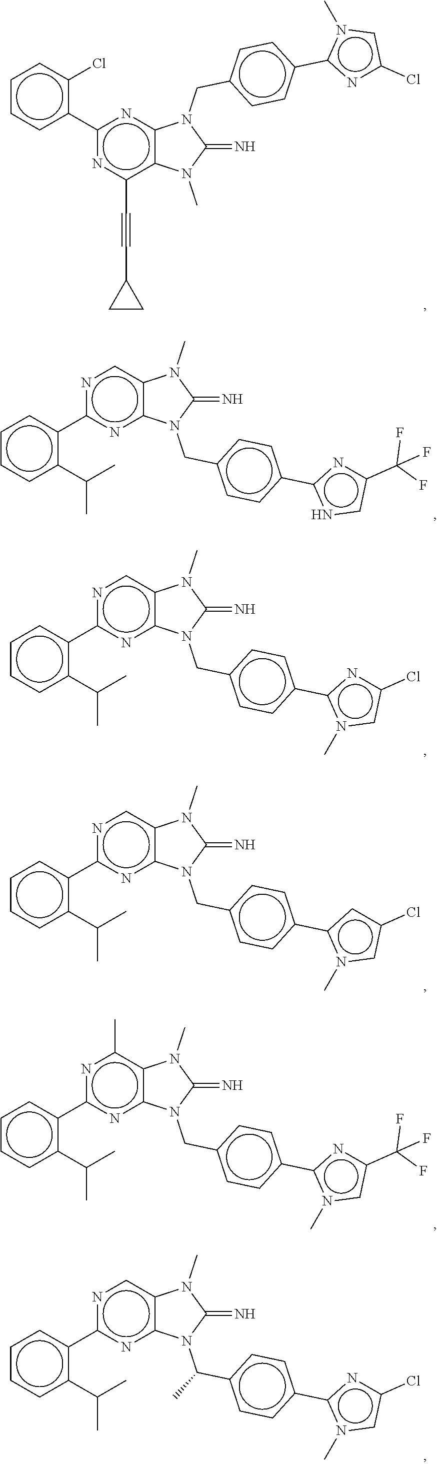

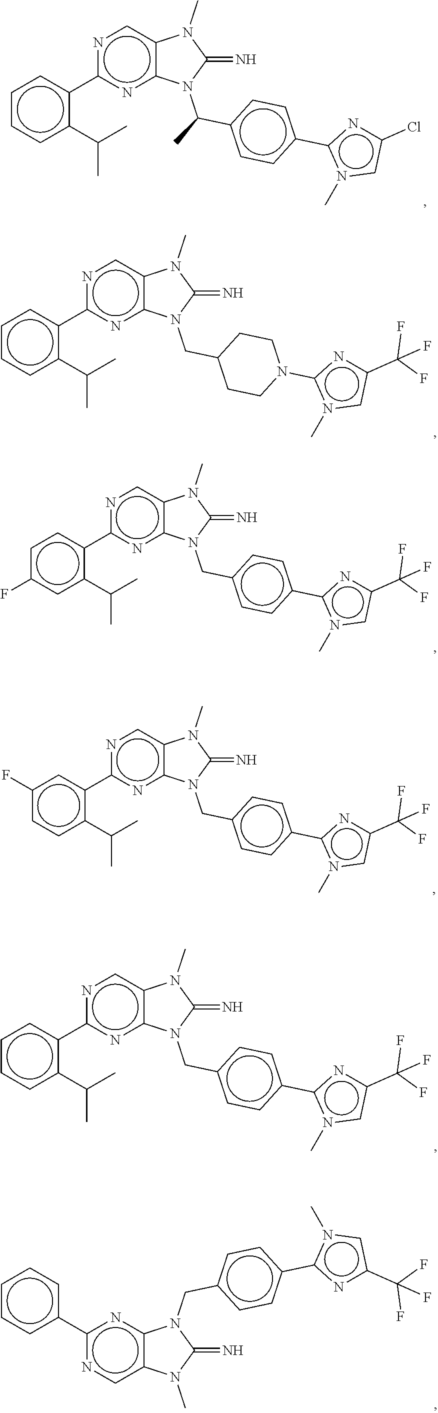

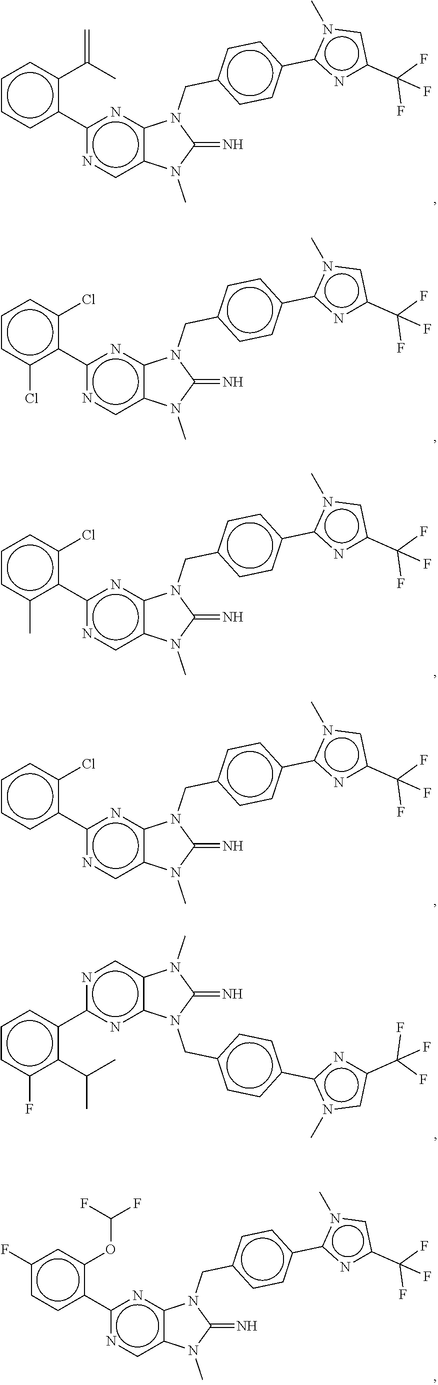

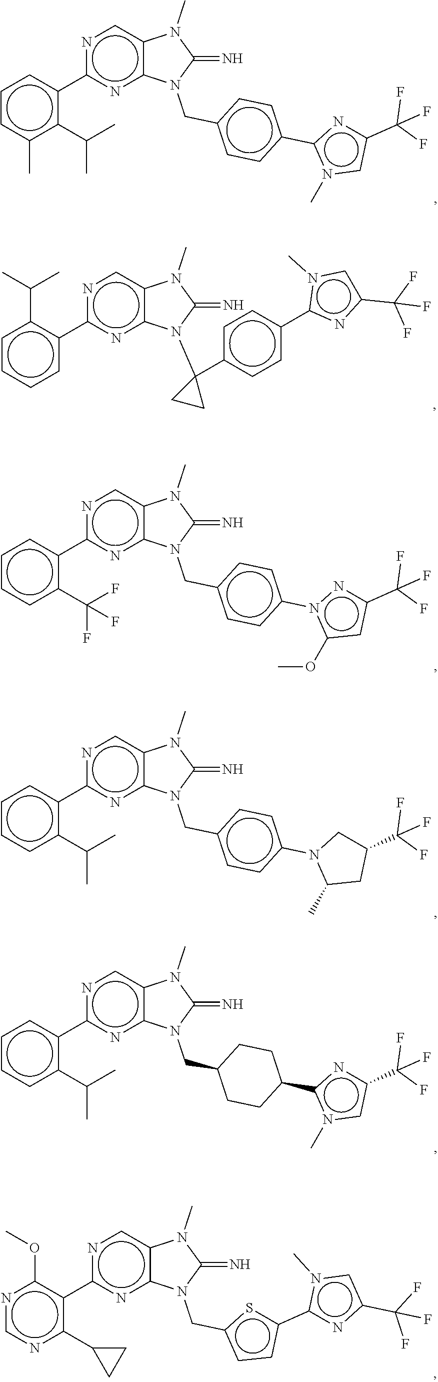

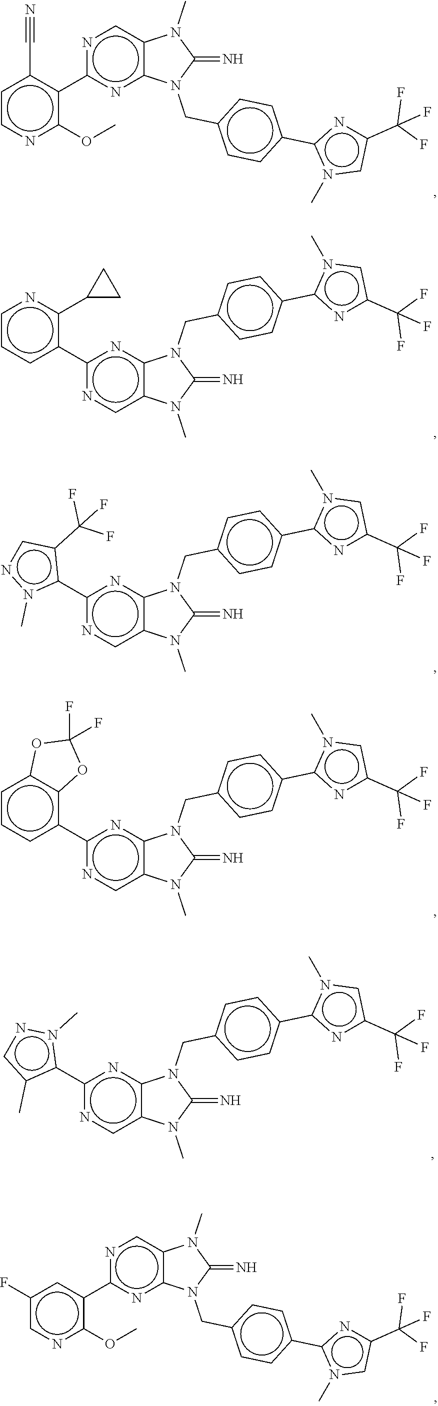

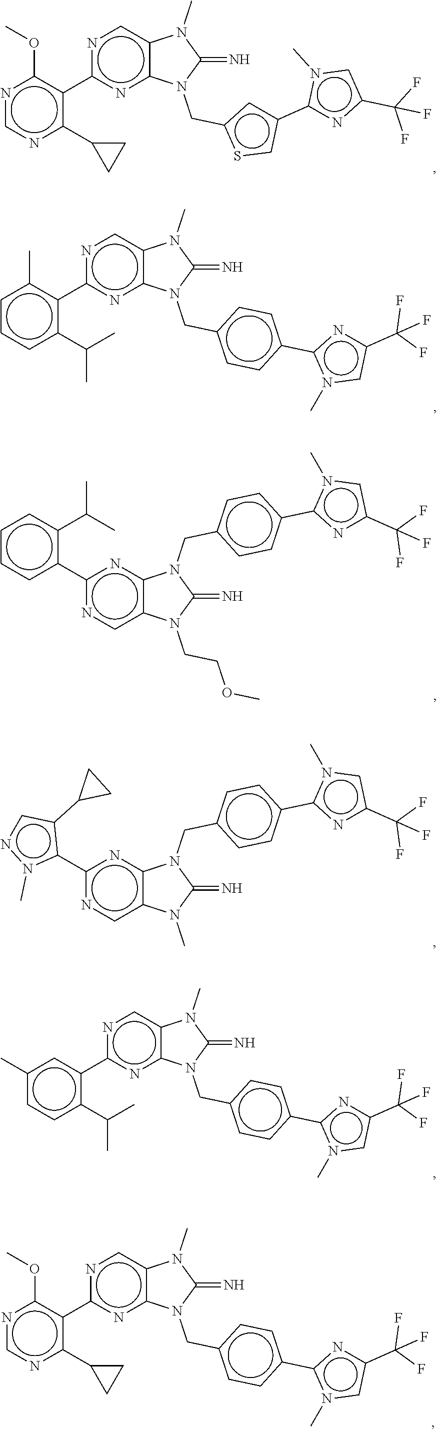

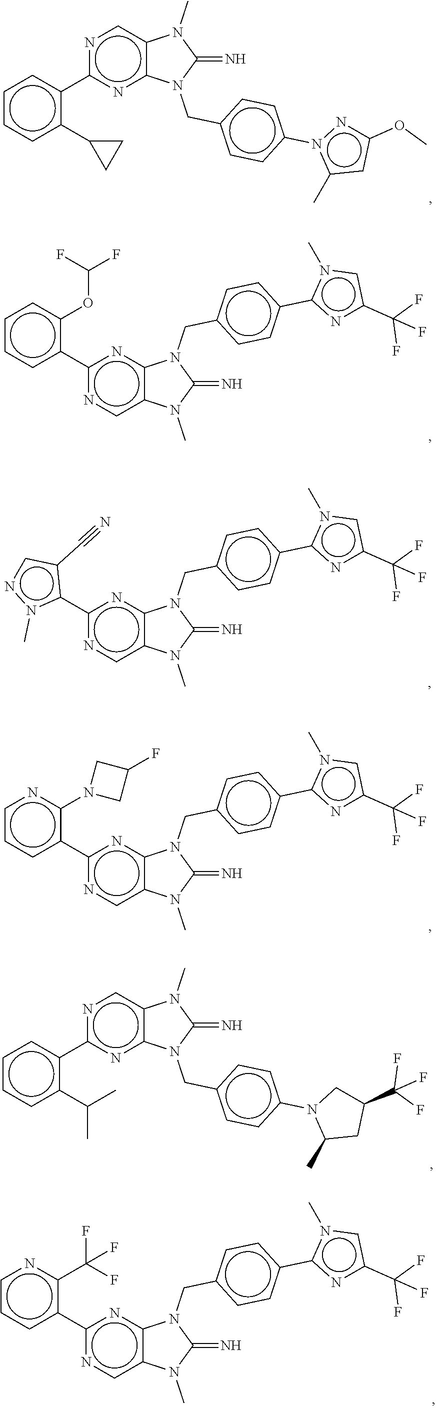

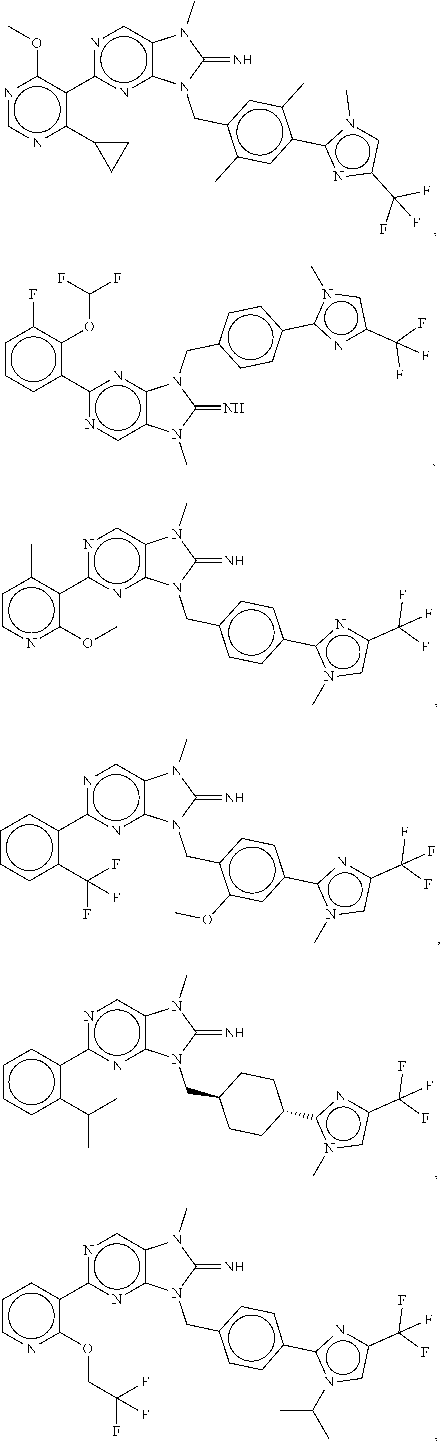

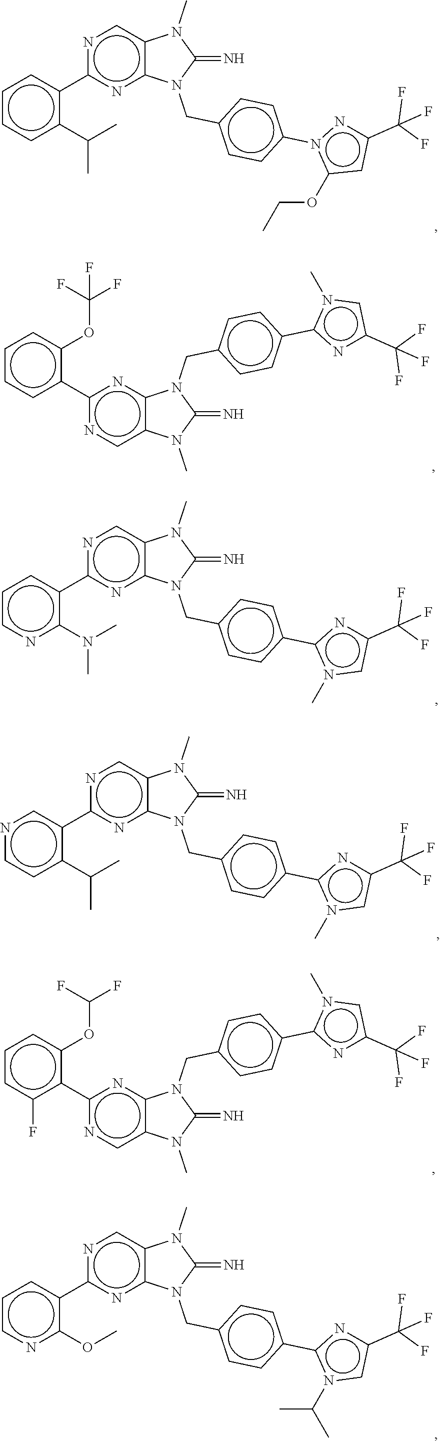

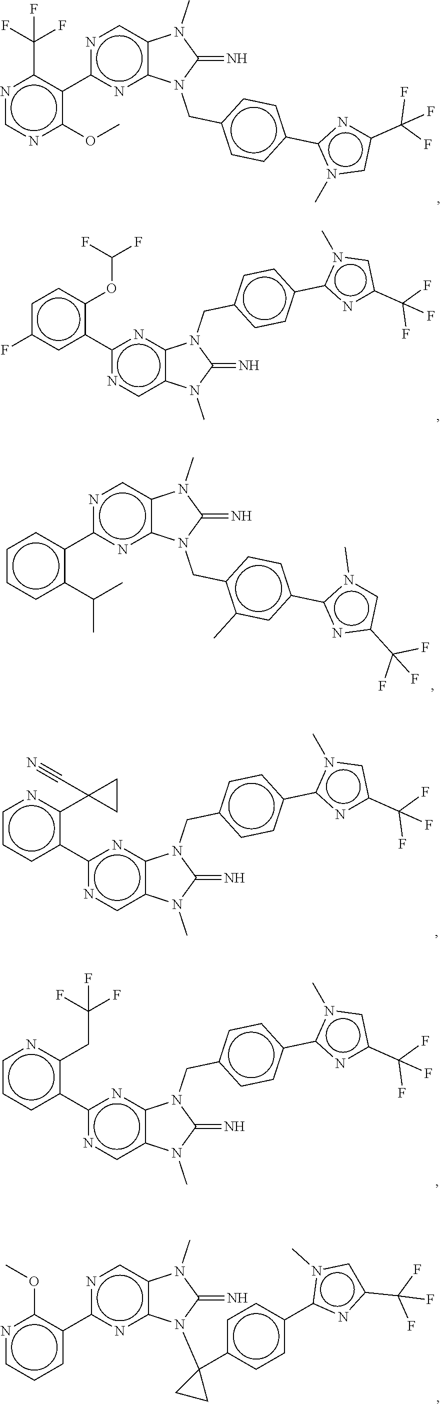

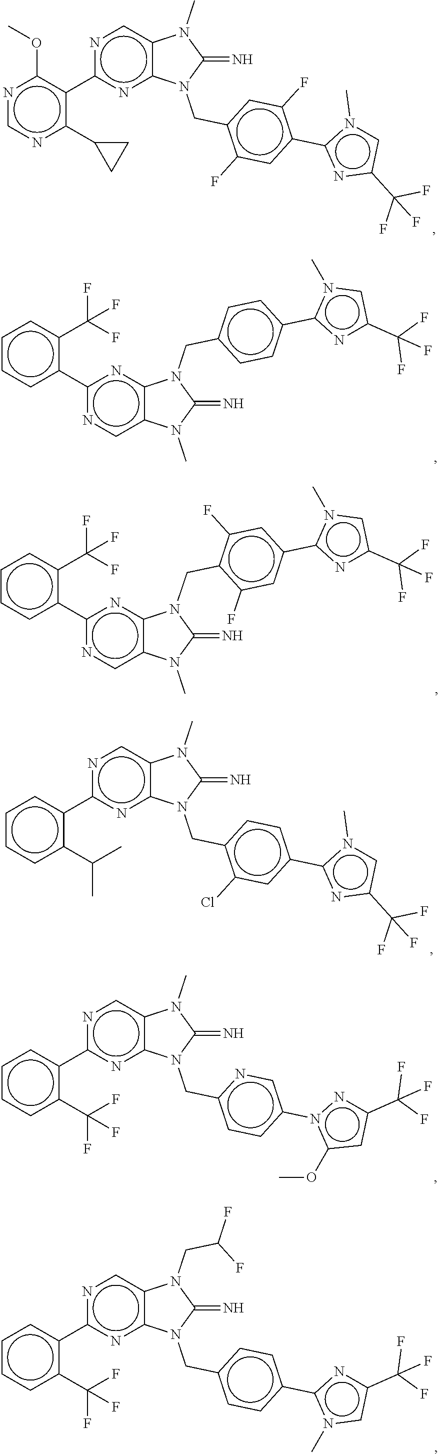

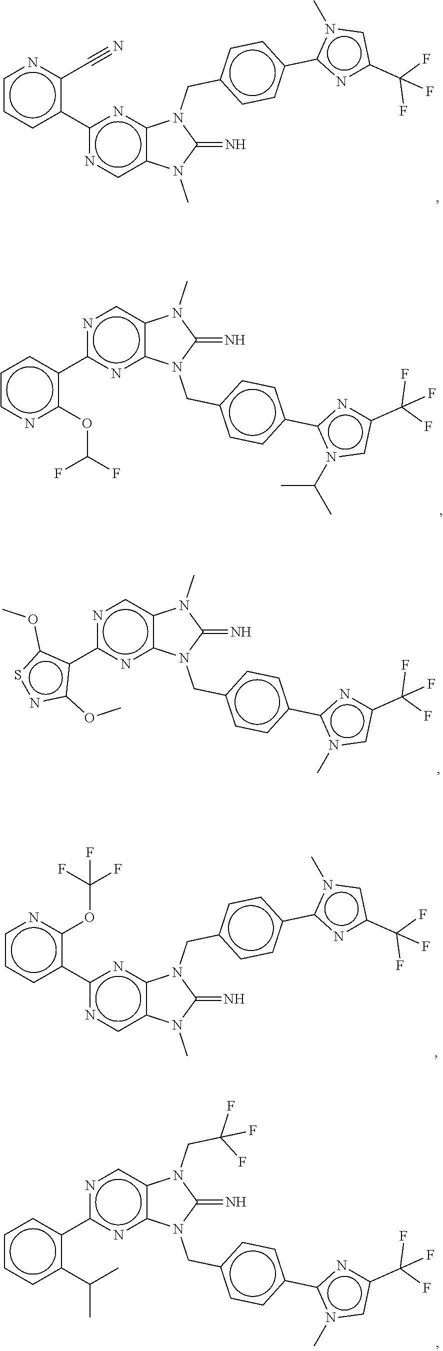

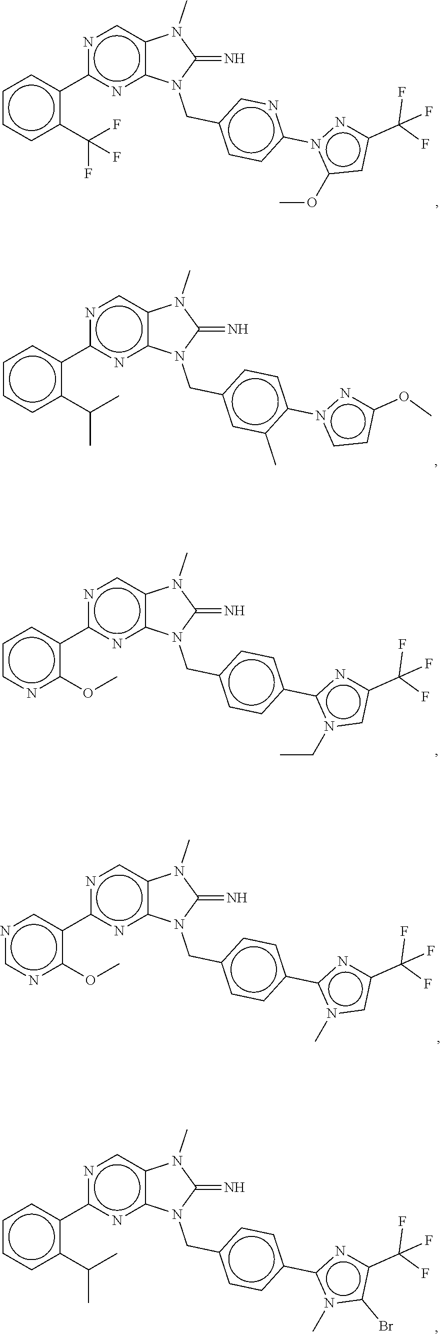

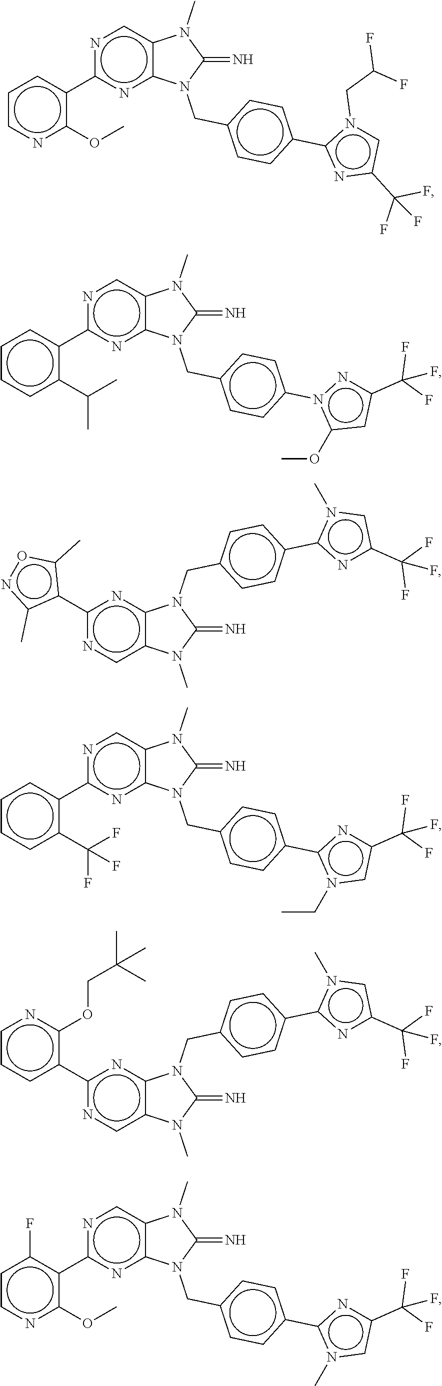

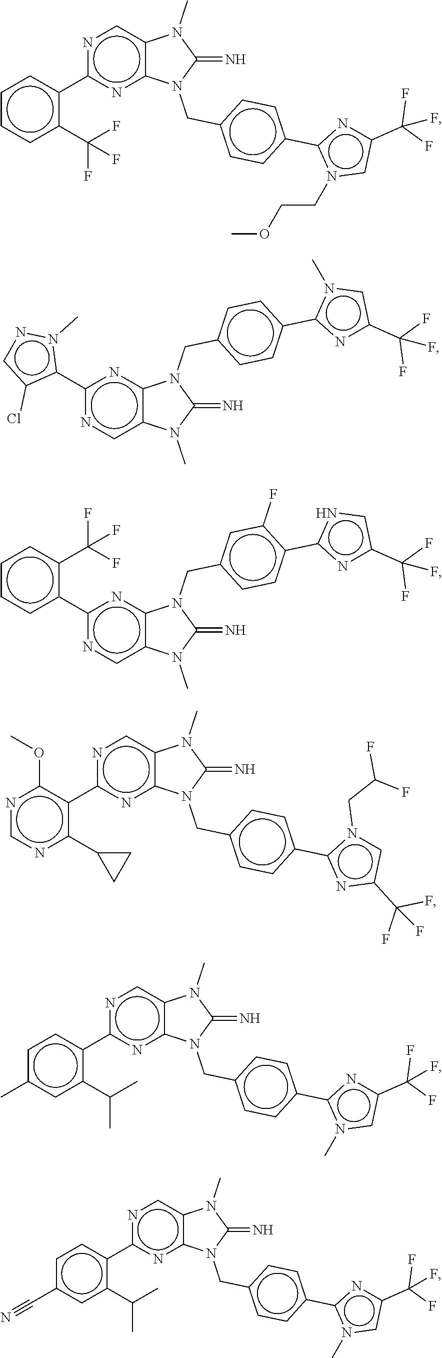

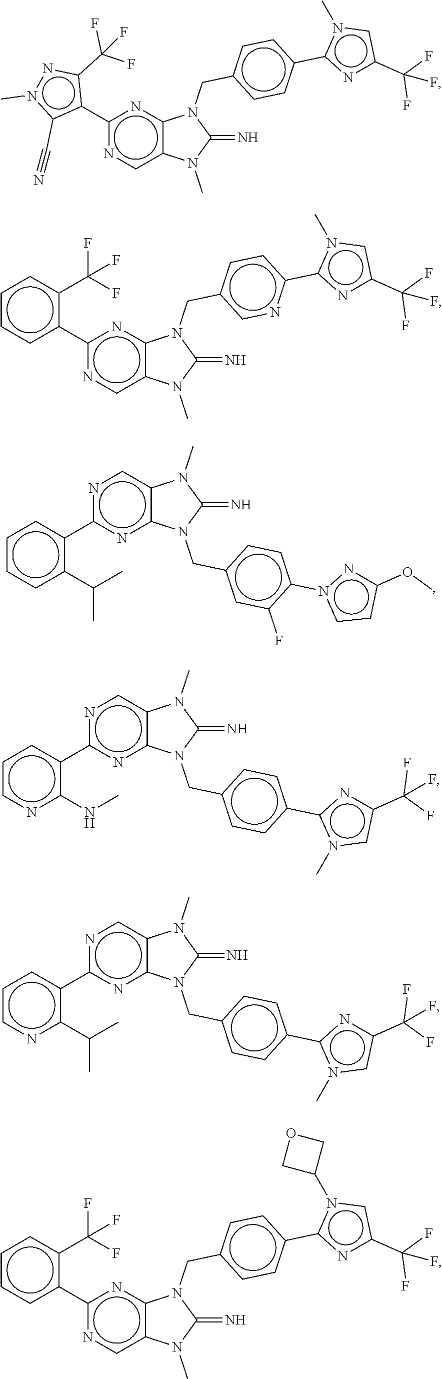

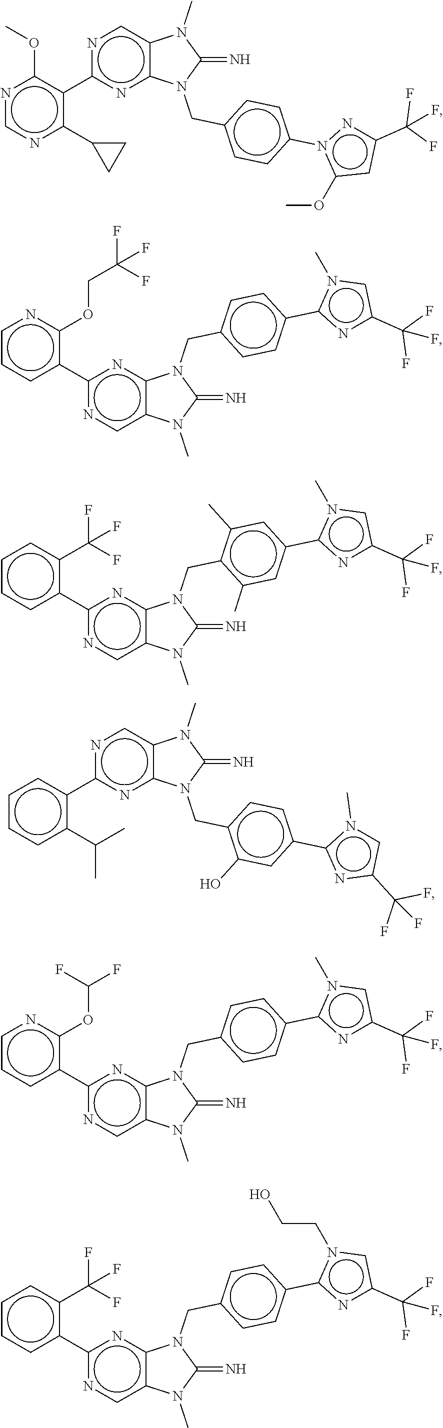

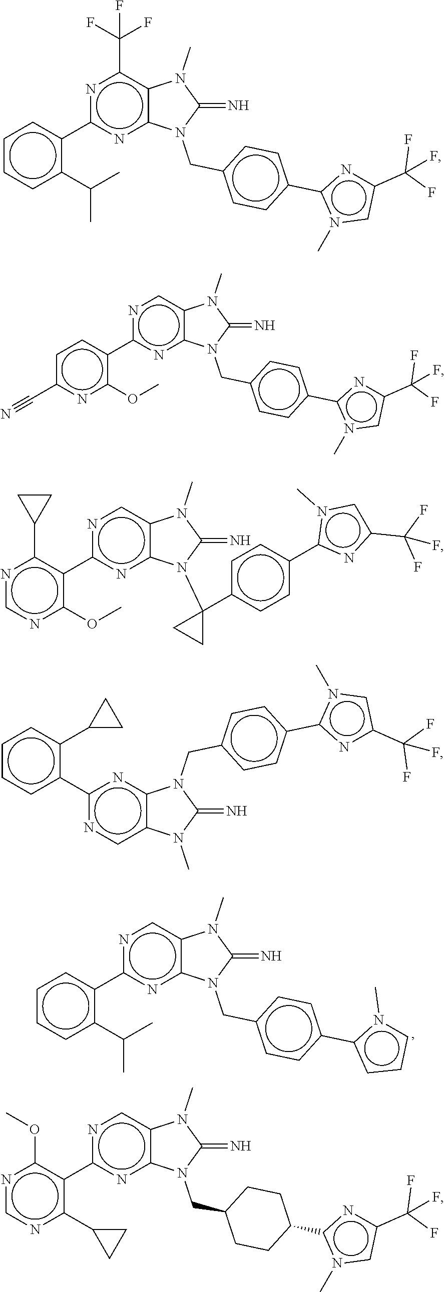

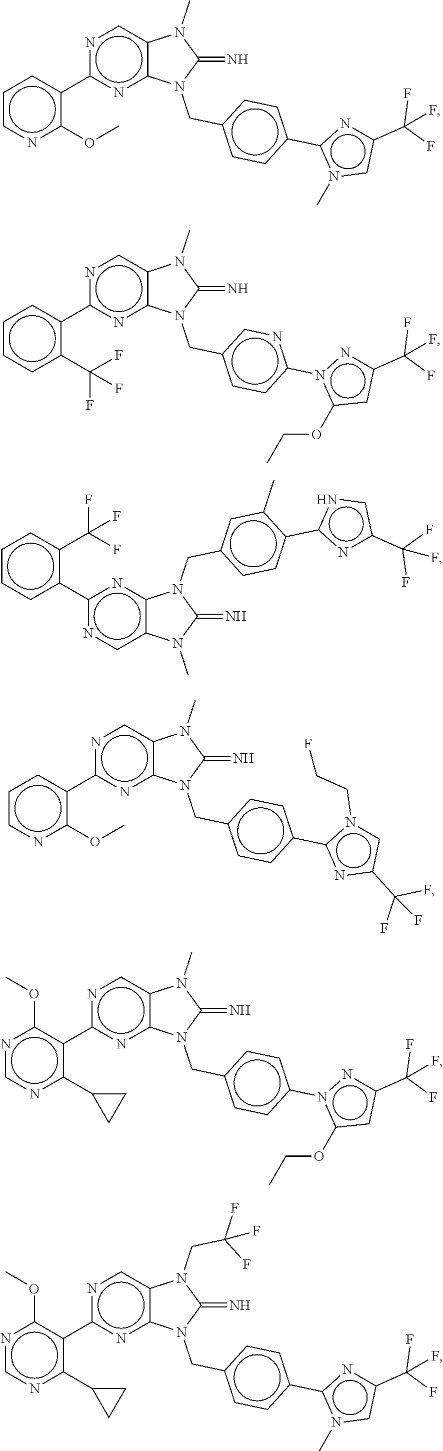

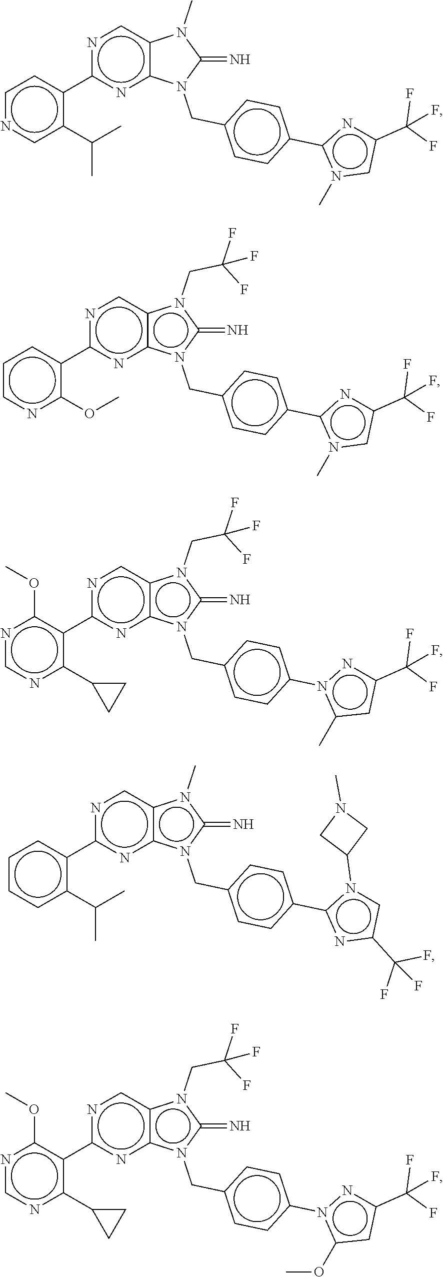

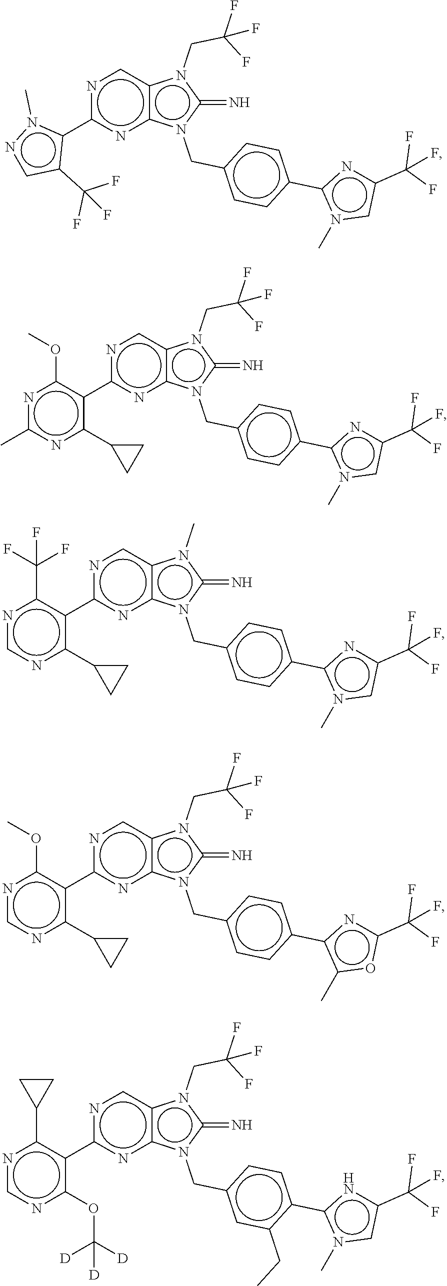

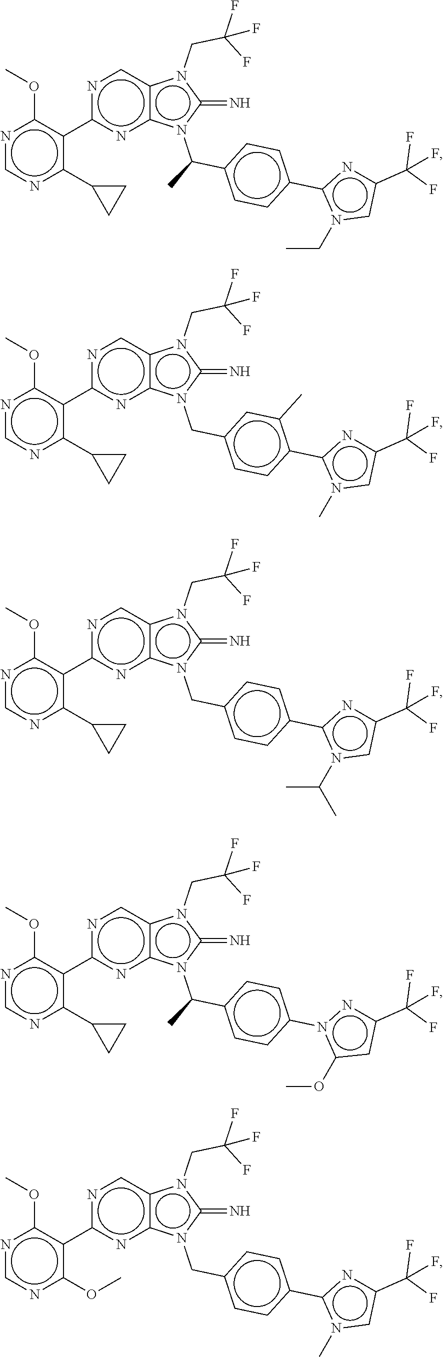

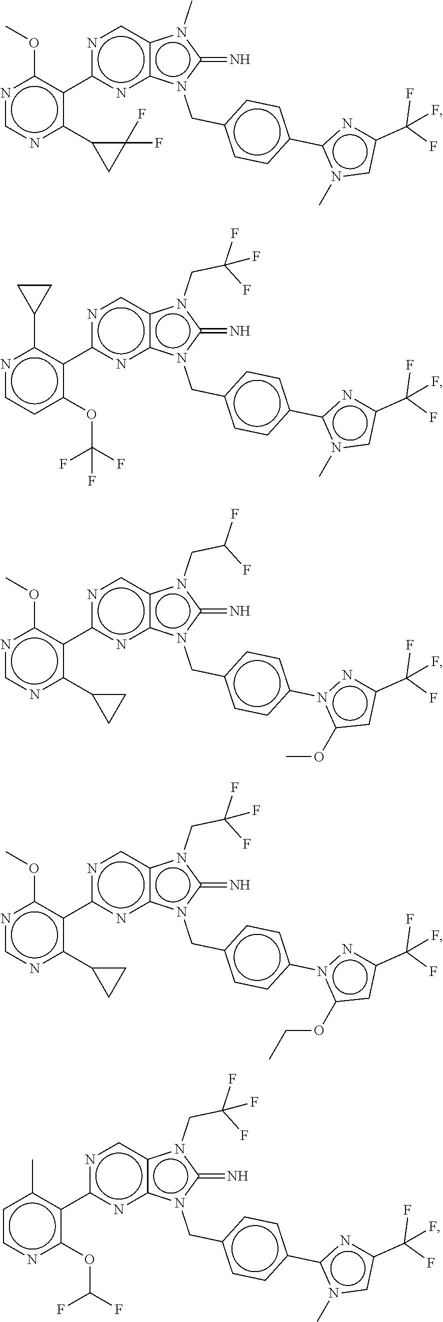

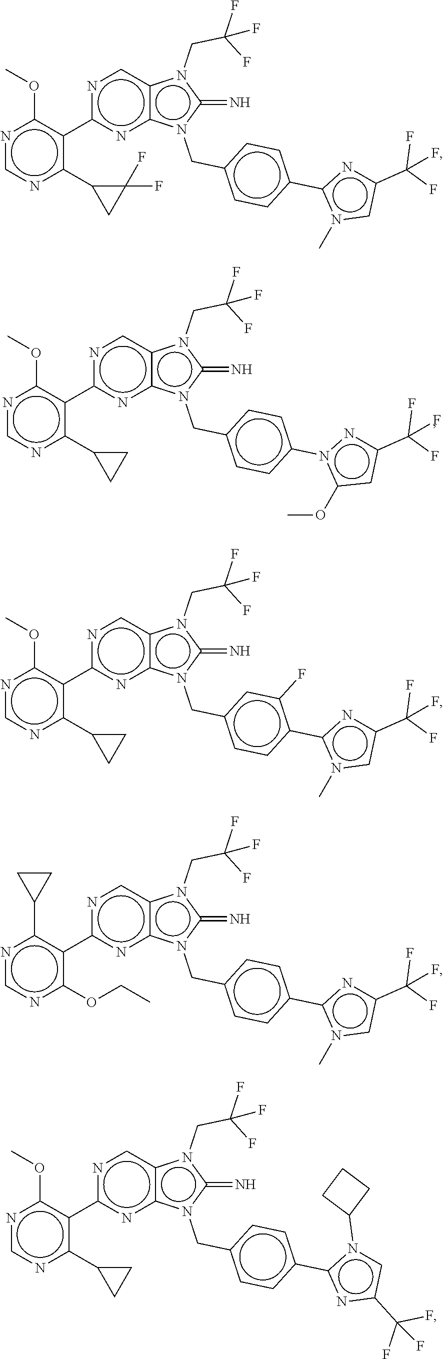

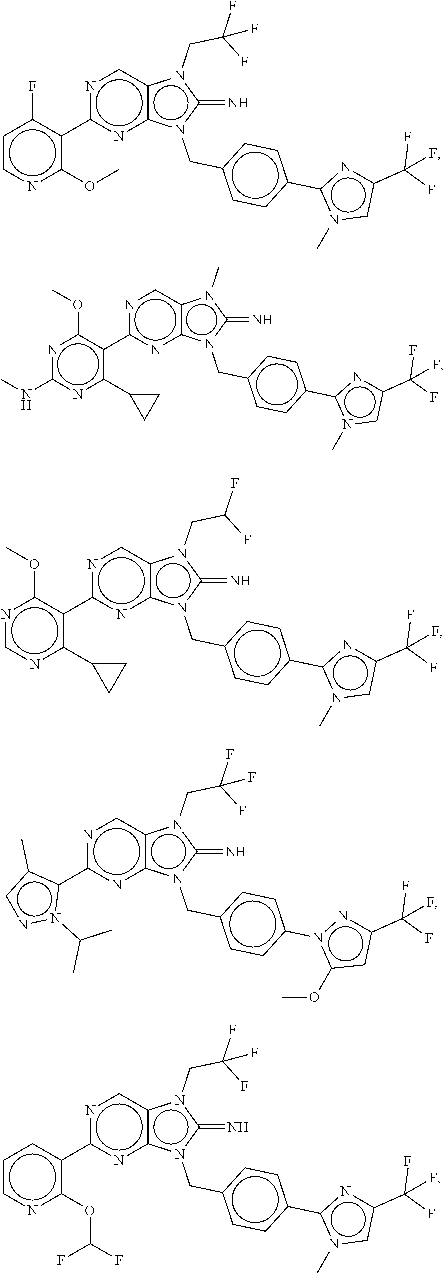

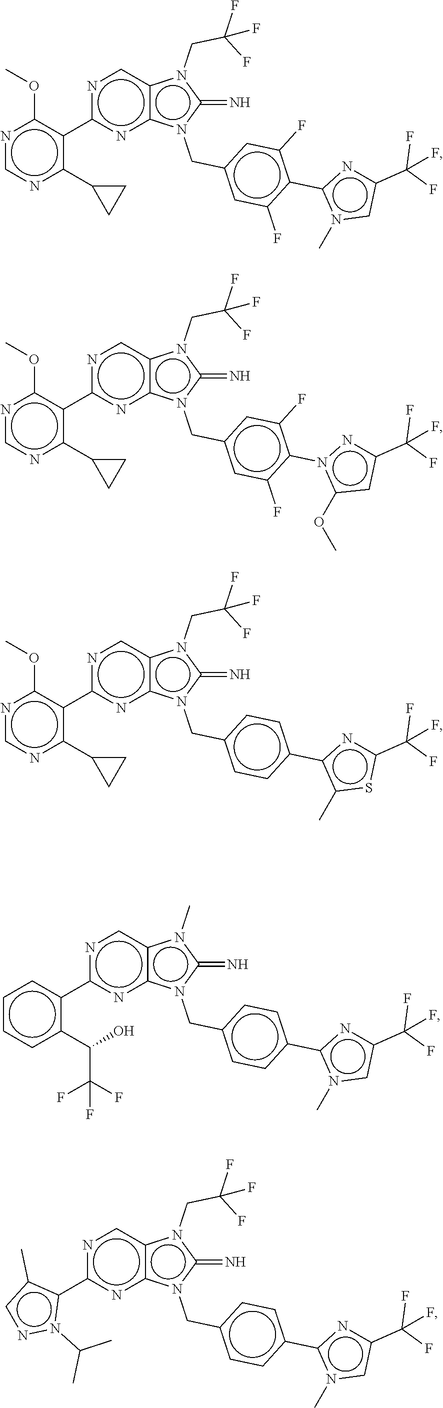

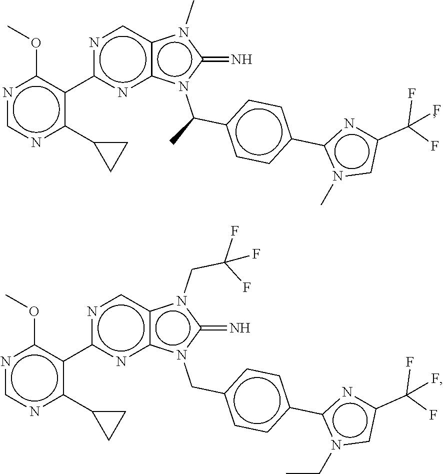

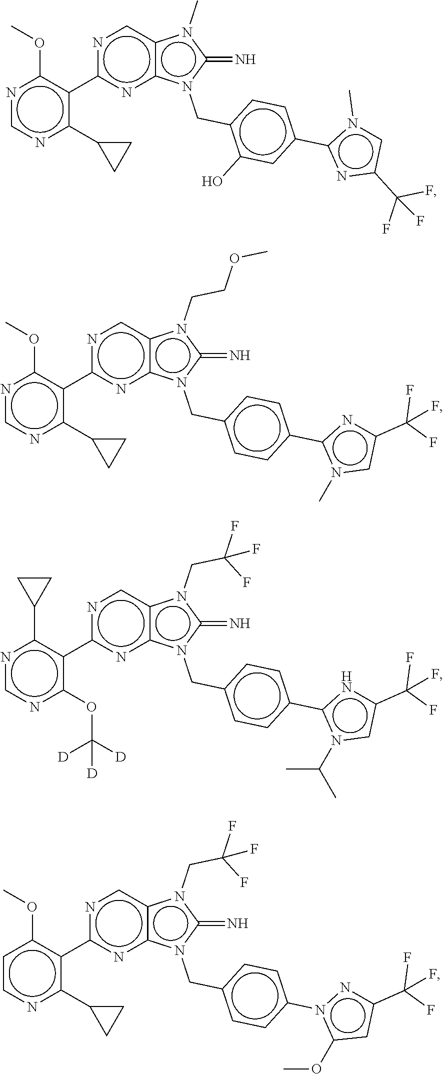

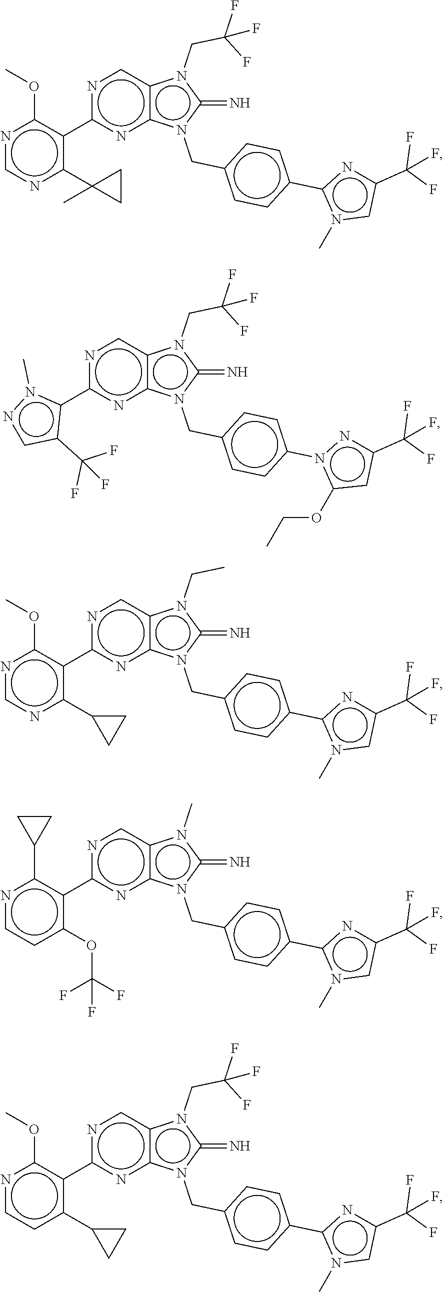

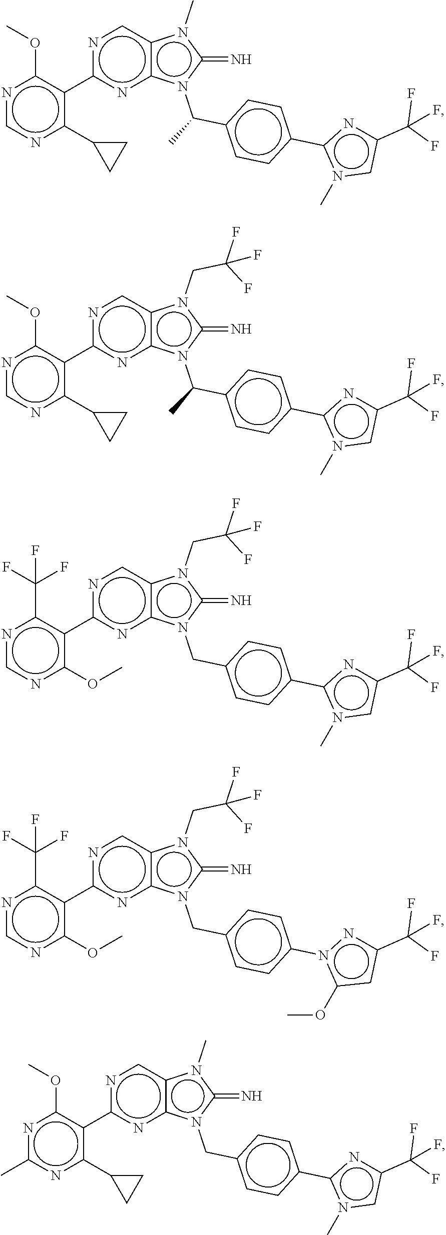

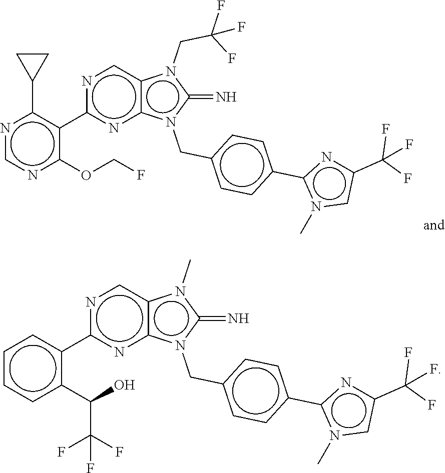

31. A pharmaceutical composition comprising a compound of claim 1 or a pharmaceutically acceptable salt, hydrate, solvate, prodrug, stereoisomer, or tautomer thereof and a pharmaceutically acceptable carrier.

32. A method for treating cancer in a patient in need thereof, comprising administering to the patient an effective amount of a compound of claim 1 or a pharmaceutically acceptable salt, hydrate, solvate, prodrug, stereoisomer, or tautomer thereof.

33-39. (canceled)

40. The method of claim 32, wherein the cancer is selected from adrenocortical carcinoma, AIDS-related lymphoma, AIDS-related malignancies, anal cancer, cerebellar astrocytoma, extrahepatic bile duct cancer, bladder cancer osteosarcoma/malignant fibrous histiocytoma, brain stem glioma, ependymoma, visual pathway and hypothalamic gliomas, breast cancer, bronchial adenomas/carcinoids, carcinoid tumors, gastrointestinal carcinoid tumors, carcinoma, adrenocortical, islet cell carcinoma, primary central nervous system lymphoma, cerebellar astrocytoma, cervical cancer, chronic lymphocytic leukemia, chronic myelogenous leukemia, clear cell sarcoma of tendon sheaths, colon cancer, colorectal cancer, cutaneous t-cell lymphoma, endometrial cancer, ependymoma, esophageal cancer, Ewing's sarcoma/family of tumors, extracranial germ cell tumors, extragonadal germ cell tumors, extrahepatic bile duct cancer, eye cancers, including intraocular melanoma, and retinoblastoma, gallbladder cancer, gastrointestinal carcinoid tumor, ovarian germ cell tumor, gestational trophoblastic tumor, hairy cell leukemia, head and neck cancer, Hodgkin's disease, hypopharyngeal cancer, hypothalamic and visual pathway glioma, intraocular melanoma, Kaposi's sarcoma, laryngeal cancer, acute lymphoblastic leukemia, acute myeloid leukemia, liver cancer, non-small cell lung cancer, small cell lung cancer, non-Hodgkin's lymphoma, Waldenstrom's macroglobulinemia, malignant mesothelioma, malignant thymoma, medulloblastoma, melanoma, intraocular melanoma, merkel cell carcinoma, metastatic squamous neck cancer with occult primary, multiple endocrine neoplasia syndrome, multiple myeloma/plasma cell neoplasm, mycosis fungoides, myelodysplastic syndrome, chronic myelogenous leukemia, myeloid leukemia, multiple myeloma, myeloproliferative disorders, nasal cavity and paranasal sinus cancer, nasopharyngeal cancer, neuroblastoma, oral cancer, oral cavity and lip cancer, oropharyngeal cancer, osteosarcoma/malignant fibrous histiocytoma of bone, ovarian cancer, ovarian low malignant potential tumor, pancreatic cancer, paranasal sinus and nasal cavity cancer, parathyroid cancer, penile cancer, pheochromocytoma, pituitary tumor, pleuropulmonary blastoma, prostate cancer, rectal cancer, renal cell (kidney) cancer, transitional cell cancer, retinoblastoma, rhabdomyosarcoma, salivary gland cancer, malignant fibrous histiocytoma of bone, soft tissue sarcoma, Sezary syndrome, skin cancer, small intestine cancer, stomach (gastric) cancer, supratentorial primitive neuroectodennal and pineal tumors, cutaneous t-cell lymphoma, testicular cancer, malignant thymoma, thyroid cancer, gestational trophoblastic tumor, urethral cancer, uterine sarcoma, vaginal cancer, vulvar cancer, and Wilms' tumor.
